(19) United States
(12) Patent Application Publication
(10) Pub. No.: US 2025/0223564 A1
(43) Pub. Date: Jul. 10, 2025

(54) HYPOIMMUNE BETA CELLS DIFFERENTIATED FROM PLURIPOTENT STEM CELLS AND RELATED USES AND METHODS

(71) Applicant: Sana Biotechnology, Inc., Seattle, WA (US)

(72) Inventors: Sonja SCHREPFER, San Mateo, CA (US); Jeffrey R. MILLMAN, St. Louis, MO (US)

(21) Appl. No.: 18/727,670

(22) PCT Filed: Jan. 9, 2023
(86) PCT No.: PCT/US2023/060341

§ 371 Date: Jul. 9, 2024

Related U.S. Application Data
(60) Provisional application No. 63/353,534, filed on Jun. 17, 2022.
(60) Provisional application No. 63/352,605, filed on Jun. 15, 2022.
(60) Provisional application No. 63/322,208, filed on Mar. 21, 2022.
(60) Provisional application No. 63/320,691, filed on Mar. 16, 2022.
(60) Provisional application No. 63/298,214, filed on Jan. 10, 2022.

(51) Int. Cl. C12N 5/071; A61K 35/39; A61P 37/06; C07K 14/705; C07K 14/74; C12N 9/22; C12N 15/11; C12N 15/86

(52) U.S. Cl. CPC C12N 5/0676; A61K 35/39; A61P 37/06; C07K 14/70539; C07K 14/70596; C12N 9/22; C12N 15/111; C12N 15/86; C12N 2310/20; C12N 2501/117; C12N 2501/15; C12N 2501/999; C12N 2506/45; C12N 2510/00; C12N 2740/15043

(57) ABSTRACT

Provided are functional modified stem cell-derived beta (β) cells (SC-beta cells) containing one or more modifications, such as genetic modifications, and related methods of their use and generation. In some embodiments, the modified cells are hypoimmunogenic cells. In some embodiments, the modified SC-beta cells are cells differentiated in vitro from a modified or hypoimmunogenic pluripotent stem cell that contains the one or more modifications. In some embodiments, the one or more modifications reduce or eliminate expression of MHC class I and/or MHC class II human leukocyte antigens and also exogenously express one or more tolerogenic factors such as CD47.

### CROSS-REFERENCE TO RELATED APPLICATIONS

This application claims priority to U.S. Provisional Patent Application No. 63/298,214 filed Jan. 10, 2022, U.S. Provisional Patent Application No. 63/320,691 filed Mar. 16, 2022, U.S. Provisional Patent Application No. 63/322,208 filed Mar. 21, 2022, U.S. Provisional Patent Application No. 63/352,605 filed Jun. 15, 2022, and U.S. Provisional Patent Application No. 63/353,534 filed Jun. 17, 2022, the contents of each of which are herein incorporated by reference in their entireties for all purposes.

### REFERENCE TO AN ELECTRONIC SEQUENCE LISTING

The contents of the electronic sequence listing (186152006640seq.xml; Size: 35,375 bytes; and Date of Creation: Jan. 6, 2023) is herein incorporated by reference in its entirety.

### FIELD

In certain aspects, the present disclosure is directed to modified or engineered stem cell-derived beta (β) cells (SC-beta cells) containing one or more genetic modification, such as genetic modifications, and related methods of their use and generation. In some embodiments, the modified cells are hypoimmunogenic cells. In some embodiments, the modified SC-beta cells are cells differentiated in vitro from a modified or hypoimmunogenic pluripotent stem cell that contains the one or more modifications. In some embodiments, the one or more modifications reduce or eliminate expression of MHC class I and/or MHC class II human leukocyte antigens and also exogenously express one or more tolerogenic factors such as CD47.

### SUMMARY

Provided herein is a method of generating a modified stem cell derived beta cell (SC-beta cell), the method comprising: (A) providing a modified pluripotent stem cell (PSC) comprising modifications that: (a) inactivate or disrupt one or more alleles of: (i) one or more major histocompatibility complex (MHC) class I molecules or one or more molecules that regulate expression of the one or more MHC class I molecules, and/or (ii) one or more MHC class II molecules or one or more molecules that regulate expression of the one or more MHC class II molecules; and (b) increase expression of one or more tolerogenic factors in the modified PSC, relative to a control or wild-type PSC; and (B) culturing the modified PSC under conditions sufficient for differentiation of the modified PSC into the modified SC-beta cell.

Provided herein is a method of generating a modified stem cell derived beta cell (SC-beta cell) the method comprising: (A) generating a modified pluripotent stem cell (PSC) comprising: (a) introducing, into a PSC, one or more modifications that inactivate or disrupt one or more alleles of: (i) one or more major histocompatibility complex (MHC) class I molecules or one or more molecules that regulate expression of the one or more MHC class I molecules, and/or (ii) one or more MHC class II molecules or one or more molecules that regulate expression of the one or more MHC class II molecules; and (b) increasing expression of one or more tolerogenic factors in the PSC, relative to a control or wild-type PSC; and (B) culturing the modified PSC under conditions sufficient for differentiation of the modified PSC into a modified SC-beta cell.

Provided herein is a method of generating a modified stem cell derived beta cell (SC-beta cell), the method comprising (A) providing a modified pluripotent stem cell (PSC) that comprises at least one modification selected from the group consisting of: (a) modifications that inactivate or disrupt one or more alleles of: (i) one or more major histocompatibility complex (MHC) class I molecules or one or more molecules that regulate expression of the one or more MHC class I molecules, and/or (ii) one or more MHC class II molecules or one or more molecules that regulate expression of the one or more MHC class II molecules; and (b) modifications that increase expression of one or more tolerogenic factors in the modified PSC, relative to a control or wild-type cell of the same cell type that does not comprise the modification; (B) culturing the modified PSC under conditions sufficient for differentiation of the modified PSC into a modified SC-beta cell; and (C) introducing one or more additional modifications into the modified SC-beta cell, wherein the one or more additional modifications comprise at least one or more other modifications of (a), (b), or (a) and (b) not present in the modified PSC.

Provided herein is a method of generating a modified stem cell derived beta cell (SC-beta cell), the method comprising (A) culturing a pluripotent stem cell (PSC) under conditions sufficient for differentiation of the PSC into a SC-beta cell; and (B) generating a modified SC-beta cells comprising: (a) introducing, into the SC-beta cell, one or more modifications that inactivate or disrupt one or more alleles of: (i) one or more major histocompatibility complex (MHC) class I molecules or one or more molecules that regulate expression of the one or more MHC class I molecules, and/or (ii) one or more MHC class II molecules or one or more molecules that regulate expression of the one or more MHC class II molecules; and (b) increasing expression of one or more tolerogenic factors in the SC-beta cell, relative to a control or wild-type SC-beta cell.

Provided herein is a method of generating a generating a modified stem cell derived beta cell (SC-beta cell), the method comprising (A) providing a modified pluripotent stem cell (PSC) comprising modifications that: (a) reduce expression of one or more major histocompatibility complex (MHC) class I molecule and/or one or more MHC class II molecule in the modified PSC, relative to a control or wild-type PSC; and (b) increase expression of one or more tolerogenic factors in the modified PSC, relative to the control or wild-type PSC; and (B) culturing the modified PSC under conditions sufficient for differentiation of the modified PSC into the modified SC-beta cell.

Provided herein is a method of generating a generating a modified stem cell derived beta cell (SC-beta cell), the method comprising (A) generating a modified pluripotent stem cell (PSC) comprising (a) reducing expression of one or more major histocompatibility complex (MHC) class I molecules and/or one or more MHC class II molecules in a PSC, relative to a control or wild-type PSC; and (b) increasing expression of one or more tolerogenic factors in the PSC, relative to the control or wild-type PSC; and (B) culturing the modified PSC under conditions sufficient for differentiation of the modified PSC into a modified SC-beta cell.

In some of any of the provided embodiments, in (a) reducing expression of the one or more MHC class I molecules and/or the one or more MHC class II molecules comprises introducing modifications that reduce expression of the one or more MHC class I molecule and/or the one or more MHC class II molecules in the modified PSC, relative to the control or wild-type PSC.

In some of any of the provided embodiments, the control or wild-type PSC is an unmodified PSC that does not comprise the modifications.

In some of any of the provided embodiments, the PSC does not comprise the modifications.

In some of any of the provided embodiments, expression of one or more MHC class I molecules and one or more MHC class II molecules is reduced in the modified PSC.

In some of any of the provided embodiments, the modifications in (a) reduce protein expression of the one or more MHC class I molecules. In some of any of the provided embodiments, the modifications in (a) reduce cell surface expression of the one or more MHC class I molecules. In some of any of the provided embodiments, the modifications in (a) reduce a function of the one or more MHC class I molecules. In some embodiments, the function is antigen presentation.

In some of any of the provided embodiments, the one or more modifications in (a) reduce cell surface trafficking of the one or more MHC class I molecules.

In some of any of the provided embodiments, the one or more MHC class I molecules is one or more human leukocyte antigen (HLA) class I molecules. In some embodiments, the one or more MHC HLA class I molecules is selected from the group consisting of HLA-A, HLA-B, and HLA-C.

In some of any of the provided embodiments, the one or more molecules that regulate cell surface protein expression of the one or more MHC class I molecules are B2M. In some embodiments, the modification that reduce expression of the one or more MHC class I molecules reduce expression of the B-2 microglobulin (B2M) gene and/or the transporter 1, ATP binding cassette subfamily B member (TAP1) gene. In some embodiments, the modifications that reduce expression of the one or more MHC class I molecules reduce expression of the B-2 microglobulin (B2M) gene. In some embodiments, the modification that reduce expression of the one or more MHC class I molecules reduce expression of the transporter 1, ATP binding cassette subfamily B member (TAP1) gene. In some embodiments, the modifications reduce expression of the B-2 microglobulin (B2M) gene and the transporter 1, ATP binding cassette subfamily B member (TAP1) gene. In some embodiments, the modifications reduce expression reduce expression of the B2M gene.

In some of any of the provided embodiments, the modification that reduces expression of the one or more MHC class I molecules reduces expression of B2M. In some of any of the provided embodiments, the modification that reduces expression of the one or more MHC class I molecules reduces mRNA expression of the B2M gene. In some of any of the provided embodiments, the modification that reduces expression of the one or more MHC class I molecules reduces protein expression of B2M. In some of any of the provided embodiments, the modification that reduces expression of the one or more MHC class I molecules comprises inactivation or disruption of one allele of the B2M gene. In some of any of the provided embodiments, the modification that reduces expression of the one or more MHC class I molecules comprises inactivation or disruption of both alleles of the B2M gene. In some of any of the provided embodiments, the modification that reduces expression of the one or more MHC class I molecules comprises inactivation or disruption of all B2M coding alleles in the cell. In some of any of the provided embodiments, the inactivation or disruption comprises an indel in the B2M gene. In some of any of the provided embodiments, the inactivation or disruption comprises a frameshift mutation or a deletion of a contiguous stretch of genomic DNA of the B2M gene. In some embodiments, the B2M gene is knocked out.

In some of any of the provided embodiments, the modification that reduces expression of the one or more MHC class I molecules reduces expression of TAP1. In some of any of the provided embodiments, the modification that reduces expression of the one or more MHC class I molecules reduces mRNA expression of the TAP1 gene. In some embodiments, the modification that reduces expression of the one or more MHC class I molecules reduce expression reduces protein expression of a protein encoded by the TAP1 gene. In some embodiments, the modification comprises inactivation or disruption of one allele of the TAP1 gene. In some embodiments, the modification comprises inactivation or disruption of both alleles of the TAP1 gene. In some embodiments, the modification comprises inactivation or disruption of all coding sequences of the TAP1 gene in the cell. In some embodiments, the inactivation or disruption comprises an indel in one allele of the TAP1 gene. In some embodiments, the inactivation or disruption comprises an indel in both alleles of the TAP1 gene. In some embodiments, the one or more modifications that reduce expression comprises a frameshift mutation or a deletion of a contiguous stretch of genomic DNA of the TAP1 gene. In some embodiments, the TAP1 gene is knocked out.

In some of any of the provided embodiments, the modifications in (a) reduce protein expression of the one or more MHC class II molecules. In some of any of the provided embodiments, the modifications in (a) reduce cell surface expression of the one or more MHC class II molecules. In some of any of the provided embodiments, the modifications in (a) reduce a function of the one or more MHC class II molecules. In some embodiments, the function is antigen presentation.

In some of any of the provided embodiments, the one or more modifications in (a) reduce cell surface trafficking of the one or more MHC class II molecules.

In some of any of the provided embodiments, the one or more MHC class II molecules is one or more human leukocyte antigen (HLA) class II molecules. In some embodiments, the one or more MHC HLA class II molecules is selected from the group consisting of HLA-DP, HLA-DQ, and/or HLA-DR.

In some of any of the provided embodiments, the one or more molecules that regulate expression of the one or more MHC class II molecules is/are selected from the group consisting of CIITA and CD74. In some embodiments, the modification that reduce expression of the one or more MHC class II molecules reduce expression of the CIITA gene and/or CD74 gene. In some embodiments, the modification is a modification that regulates expression of the one or more MHC class II molecules, and the modification inactivates or disrupts one or more alleles of CIITA. In some embodiments, the modifications that reduce expression of the one or more MHC class II molecules reduce expression of the CITTA gene. In some embodiments, the modification that reduce expression of the one or more MHC class II molecules reduce expression of the CD74 gene. In some embodiments, the modifications reduce expression of the CIITA gene and the CD74 gene.

In some of any of the provided embodiments, the modification that reduces expression of the one or more MHC class II molecules reduces expression of CD74. In some of any of the provided embodiments, the modification that reduces expression of the one or more MHC class II molecules reduces mRNA expression of the CD74 gene. In some embodiments, the modification that reduces expression of the one or more MHC class II molecules reduce expression reduces protein expression of a protein encoded by the CD74 gene. In some embodiments, the modification comprises inactivation or disruption of one allele of the CD74 gene. In some embodiments, the modification comprises inactivation or disruption of both alleles of the CD74 gene. In some embodiments, the modification comprises inactivation or disruption of all coding sequences of the CD74 gene in the cell. In some embodiments, the inactivation or disruption comprises an indel in one allele of the CD74 gene. In some embodiments, the inactivation or disruption comprises an indel in both alleles of the CD74 gene. In some embodiments, the one or more modifications that reduce expression comprises a frameshift mutation or a deletion of a contiguous stretch of genomic DNA of the CD74 gene. In some embodiments, the CD74 gene is knocked out.

In some of any of the provided embodiments, the modification that reduces expression of the one or more MHC class II molecules comprises reduced expression of CIITA. In some of any of the provided embodiments, the modification that reduces expression of the one or more MHC class II molecules reduces mRNA expression of the CIITA gene. In some of any of the provided embodiments, the modification that reduces expression of the one or more MHC class II molecules reduces protein expression of CIITA. In some of any of the provided embodiments, the modification that reduces expression of the one or more MHC class II molecules comprises inactivation or disruption of one allele of the CIITA gene. In some of any of the provided embodiments, the modification that reduces expression of the one or more MHC class II molecules comprises inactivation or disruption of both alleles of the CIITA gene. In some of any of the provided embodiments, the modification that reduces expression of the one or more MHC class II molecules comprises or inactivation or disruption of all CIITA coding alleles in the cell. In some of any of the provided embodiments, the inactivation or disruption comprises an indel in the CIITA gene. In some of any of the provided embodiments, the inactivation or disruption is a frameshift mutation or a deletion of a contiguous stretch of genomic DNA of the CIITA gene. In some embodiments, the CIITA gene is knocked out.

In some of any of the provided embodiments, expression of all MHC class I molecules and all MHC class II molecules is reduced in the modified PSC. In some embodiments, expression of HLA-A, HLA-B, HLA-C, HLA-DP, HLA-DQ, and HLA-DR are reduced in the modified PSC.

In some of any of the provided embodiments, the one or more tolerogenic factors is selected from the group consisting of CD47, CD27, CD200, HLA-C, HLA-E, HLA-E heavy chain, HLA-G, PD-L1, IDO1, CTLA4-Ig, Cl-Inhibitor, IL-10, IL-35, FASL, CCL21, MFGE8, and SERPINB9. In some of any of the provided embodiments, at least one of the one or more tolerogenic factors is CD47. In some of any of the provided embodiments, the one or more tolerogenic factors is CD47. In some of any of the provided embodiments, at least one of the one or more tolerogenic factors is PD-L1. In some of any of the provided embodiments, the one or more tolerogenic factors is PD-L1. In some of any of the provided embodiments, at least one of the one or more tolerogenic factors is HLA-E. In some of any of the provided embodiments, the one or more tolerogenic factors is HLA-E. In some of any of the provided embodiments, at least one of the one or more tolerogenic factors is HLA-G. In some of any of the provided embodiments, the one or more tolerogenic factors is HLA-G.

In some of any of the provided embodiments, increasing expression of the one or more tolerogenic factors comprises introducing a modification that increases expression of the one or more tolerogenic factors in the modified PSC, relative to the control or wild-type PSC. In some of any of the provided embodiments, the modification to increase expression of the one or more tolerogenic factors comprises an exogenous polynucleotide encoding the one or more tolerogenic factors.

In some of any of the provided embodiments, the exogenous polynucleotide encoding the one or more tolerogenic factors is integrated into the genome of the modified PSC. In some of any of the provided embodiments, the exogenous polynucleotide is integrated by non-targeted insertion into the genome of the modified PSC. In some embodiments, the non-targeted integration is by introduction of the exogenous polynucleotide into the cell using a lentiviral vector. In some of any of the provided embodiments, the exogenous polynucleotide is integrated by targeted insertion into a target genomic locus of the cell. In some embodiments, the targeted insertion is by nuclease-mediated gene editing with homology-directed repair.

In some of any of the provided embodiments, increasing expression of the one or more tolerogenic factors comprises introducing a modification that increases expression of the one or more tolerogenic factors in the modified SC-beta cell, relative to the control or wild-type beta cell. In some of any of the provided embodiments, the modification to increase expression of the one or more tolerogenic factors comprises an exogenous polynucleotide encoding the one or more tolerogenic factors.

In some of any of the provided embodiments, the exogenous polynucleotide encoding the one or more tolerogenic factors is integrated into the genome of the modified SC-beta cell. In some of any of the provided embodiments, the exogenous polynucleotide is integrated by non-targeted insertion into the genome of the modified SC-beta cell. In some embodiments, the non-targeted integration is by introduction of the exogenous polynucleotide into the cell using a lentiviral vector. In some of any of the provided embodiments, the exogenous polynucleotide is integrated by targeted insertion into a target genomic locus of the cell. In some embodiments, the targeted insertion is by nuclease-mediated gene editing with homology-directed repair.

Also provided herein is a method of generating a modified stem cell derived beta cell (SC-beta cell), the method comprising: (A) providing a modified pluripotent stem cell (PSC) comprising knock out of the B2M gene, knock out of the CIITA gene, and an exogenous polynucleotide encoding CD47 protein, relative to a control or wild-type PSC; and (B) culturing the modified PSC under conditions sufficient for differentiation of the modified PSC into the modified SC-beta cell. In some embodiments, the modified PSC has the phenotype B2M^{indel/indel}; CIITA^{indel/indel}; CD47tg. In some of any embodiments, the modified PSC further comprises a modification to increase expression of an exogenous suicide gene. In some of any embodiments, the modified SC-beta cell further comprises a modification to increase expression of an exogenous suicide gene.

Also provided herein is a method of generating a modified stem cell derived beta cell (SC-beta cell), the method comprising: (A) providing a pluripotent stem cell (PSC); (B) culturing the PSC under conditions sufficient for differentiation of the PSC into a SC-beta cell; and (C) generating a modified SC-beta cell from the SC-beta cell by introducing modifications, into the SC-beta cell to knock out the B2M gene and to knock out the CIITA gene, and introducing an exogenous polynucleotide encoding CD47 protein. In some embodiments, the modified SC-beta cell has the phenotype B2M^{indel/indel}; CIITA^{indel/indel}; CD47tg. In some of any embodiments, the modified SC-beta cell further comprises a modification to increase expression of an exogenous suicide gene. In some of any embodiments, the modified SC-beta cell further comprises a modification to increase expression of an exogenous suicide gene.

Also provided herein is a method of generating a modified stem cell derived beta cell (SC-beta cell), the method comprising: (A) providing a modified pluripotent stem cell (PSC) comprising knock out of the B2M gene, knock out of the CIITA gene, an exogenous polynucleotide encoding CD47 protein, and an exogenous polynucleotide encoding a suicide gene, relative to a control or wild-type PSC; and (B) culturing the modified PSC under conditions sufficient for differentiation of the modified PSC into the modified SC-beta cell. In some embodiments, the modified PSC has the phenotype B2M^{indel/indel}; CIITA^{indel/indel}, CD47tg; suicide genetg.

Also provided herein is a method of generating a modified stem cell derived beta cell (SC-beta cell), the method comprising: (A) providing a pluripotent stem cell (PSC); (B) culturing the PSC under conditions sufficient for differentiation of the PSC into a SC-beta cell; and (C) generating a modified SC-beta cell from the SC-beta cell by introducing modifications, into the SC-beta cell to knock out the B2M gene and to knock out the CIITA gene, and introducing an exogenous polynucleotide encoding CD47 protein, and an exogenous polynucleotide encoding a safety switch. In some embodiments, the modified SC-beta cell has the phenotype B2M^{indel/indel}; CIITA^{indel/indel}; CD47tg; safety switch (e.g., suicide gene) transgene. In some of any embodiments provided herein, the exogenous polynucleotide encoding CD47 is integrated by non-targeted insertion into the genome of the modified SC-beta cell, optionally by introduction of the exogenous polynucleotide into the cell using a lentiviral vector.

In some embodiments of any of the methods of generating a modified SC-beta cell herein, the modified SC-beta cell comprises an exogenous polynucleotide encoding a suicide gene or suicide switch. In some embodiments, the suicide gene is selected from the group consisting of cytosine deaminase (CyD), herpesvirus thymidine kinase (HSV-Tk), an inducible caspase 9 (iCaspase9), and rapamycin-activated caspase 9 (rapaCasp9). In some embodiments, the suicide gene or suicide switch and genes associated with the suicide gene or the safety switch are expressed from a bicistronic cassette integrated into the genome of the modified SC-beta cell. In some embodiments, the suicide gene or suicide switch and the one or more tolerogenic factors are expressed from a bicistronic cassette integrated into the genome of the modified SC-beta cell. In some embodiments, the bicistronic cassette is integrated by non-targeted insertion into the genome of the modified SC-beta cell. In some embodiments, the bicistronic cassette is integrated by targeted insertion into a target genomic locus of the modified SC-beta cell. In some embodiments, the one or more tolerogenic factors is CD47.

In some of any of the provided embodiments, suicide gene is selected from the group consisting of cytosine deaminase (CyD), herpesvirus thymidine kinase (HSV-Tk), an inducible caspase 9 (iCaspase9), and rapamycin-activated caspase 9 (rapaCasp9).

In some of any of the provided embodiments, the suicide gene and the one or more tolerogenic factors are expressed from a bicistronic cassette integrated into the genome of the modified PSC. In some embodiments, the one or more tolerogenic factor is or comprises CD47 and the suicide gene and the CD47 are expressed from a bicistronic cassette integrated into the genome of the modified PSC. In some of any of the provided embodiments, the bicistronic cassette is integrated by non-targeted insertion into the genome of the modified PSC. In some embodiments, the non-targeted integration is by introduction of the exogenous polynucleotide into the cell using a lentiviral vector. In some of any of the provided embodiments, the bicistronic cassette is integrated by targeted insertion into a target genomic locus of the cell. In some embodiments, the targeted insertion is by nuclease-mediated gene editing with homology-directed repair.

In some of any of the provided embodiments, the safety switch (e.g., suicide gene) and the one or more tolerogenic factors are expressed from a bicistronic cassette integrated into the genome of the modified cell. In some embodiments, the safety switch and CD47 are expressed from a bicistronic cassette integrated into the genome of the modified cell. In some of any of the provided embodiments, the bicistronic cassette is integrated by non-targeted insertion into the genome of the modified SC-beta cell. In some embodiments, the non-targeted integration is by introduction of the exogenous polynucleotide into the cell using a lentiviral vector. In some of any of the provided embodiments, the bicistronic cassette is integrated by targeted insertion into a target genomic locus of the cell. In some embodiments, the targeted insertion is by nuclease-mediated gene editing with homology-directed repair.

In some of any of the provided embodiments, the target genomic locus is a safe harbor locus, a B2M gene locus, a CIITA gene locus, or a CD142 gene locus. In some of any of the provided embodiments, the safe harbor locus is selected from the group consisting of: a CCR5 gene locus, a CXCR4 gene locus, a PPP1R12C (also known as AAVS1) gene, an albumin gene locus, a SHS231 locus, a CLYBL gene locus, and a ROSA26 gene locus.

In some of any of the provided embodiments, the modified PSC comprises a modification that reduces expression of CD142, relative to the control or wild-type PSC. In some of any of the provided embodiments, the modification reduces mRNA expression of the CD142 gene. In some of any of the provided embodiments, the modification reduces protein expression of CD142. In some of any of the provided embodiments, the modification comprises inactivation or disruption of one allele of the CD142 gene. In some of any of the provided embodiments, the modification comprises inactivation or disruption of both alleles of the CD142 gene. In some of any of the provided embodiments, the modification comprises inactivation or disruption of all CD142 coding alleles in the cell. In some of any of the provided embodiments, the inactivation or disruption comprises an indel in the CD142 gene. In some of any of the provided embodiments, the inactivation or disruption is a frameshift mutation or a deletion of a contiguous stretch of genomic DNA of the CD142 gene.

In some of any of the provided embodiments, the modified PSC comprises a modification that increases expression of one or more complement inhibitors selected from the group consisting of CD46, CD59, CD55 and CD35, relative to the control or wild-type PSC. In some of any of the provided embodiments, the modification to increase expression of the one or more complement inhibitors comprises at least one exogenous polynucleotide selected from the group consisting of an exogenous polynucleotide encoding CD46, an exogenous polynucleotide encoding CD59, an exogenous polynucleotide encoding CD55 and an exogenous polynucleotide encoding CD35. In some of any of the provided embodiments, the one or more complement inhibitors is CD46 and CD59. In some of any of the provided embodiments, the one or more complement inhibitor is CD46, CD59 and CD55. In some of any of the provided embodiments, the at least one exogenous polynucleotide is integrated by non-targeted insertion into the genome of the modified PSC. In some embodiments the non-targeted insertion is by introduction of the exogenous polynucleotide into the cell using a lentiviral vector. In some of any of the provided embodiments, the at least one exogenous polynucleotide is integrated by targeted insertion into a target genomic locus of the cell. In some embodiments, the targeted insertion is by nuclease-mediated gene editing with homology-directed repair. In some of any of the provided embodiments, the target genomic locus is a safe harbor locus, a B2M gene locus, a CIITA gene locus, or a CD142 gene locus. In some of any of the provided embodiments, the safe harbor locus is selected from the group consisting of: a CCR5 gene locus, a CXCR4 gene locus, a PPP1R12C (also known as AAVS1) gene, an albumin gene locus, a SHS231 locus, a CLYBL gene locus, and a ROSA26 gene locus.

In some of any of the provided embodiments, the culturing the PSC under conditions sufficient for differentiation of the PSC into the SC-beta cell comprises one or more of: (i) contacting the PSC with a TGFbeta/Activin agonist and/or, a glycogen synthase kinase 3 (GSK) inhibitor and/or WNT agonist for an amount of time sufficient to form a definitive endoderm cell; (ii) contacting a definitive endoderm cell differentiated from the PSC with a FGFR2b agonist for an amount of time sufficient to form a primitive gut tube cell; (iii) contacting a primitive gut tube cell differentiated from the PSC with a retinoic acid receptor (RAR) agonist, a rho kinase inhibitor, a Smoothened antagonist, a FGFR2b agonist, a protein kinase C activator, and/or a BMP type 1 receptor inhibitor for an amount of time sufficient to form an early pancreas progenitor cell; (iv) incubating an early pancreas progenitor cell differentiated from the PSC for at least about 3 days and contacting the early pancreas progenitor cell with a rho kinase inhibitor, a TGFbeta-/Activin agonist, a Smoothened antagonist, an FGFR2b agonist, a RAR agonist, a protein kinase C activator, and/or a BMP type 1 receptor inhibitor for an amount of time sufficient to form a pancreatic progenitor cell, wherein the RAR agonist concentration is less than the RAR agonist concentration in step (iii); (v) contacting a pancreatic progenitor cell differentiated from the PSC with an Alk5 inhibitor/TGFbeta receptor inhibitor, a gamma secretase inhibitor, a Smoothened antagonist, an Erbb1 (EGFR) or Erbb4 agonist, a thyroid hormone, and/or a RAR agonist for an amount of time sufficient to form an endoderm cell, wherein during at least a portion of the contacting in (v) comprises depolymerizing the actin cytoskeleton at a time and for an amount of time sufficient to increase differentiation efficiency; and/or (vi) incubating an endoderm cell differentiated from the PSC for an amount of time in serum-free media sufficient to form a beta cell.

In some of any of the provided embodiments, the culturing the modified PSC under conditions sufficient for differentiation of the modified PSC into the modified SC-beta cell comprises one or more of (i) contacting the modified PSC with a TGF/Activin agonist or a glycogen synthase kinase 3 (GSK) inhibitor or WNT agonist for an amount of time sufficient to form a definitive endoderm cell; (ii) contacting a definitive endoderm cell differentiated from the modified PSC with a FGFR2b agonist for an amount of time sufficient to form a primitive gut tube cell; (iii) contacting a primitive gut tube cell differentiated from the modified PSC with an RAR agonist, and optionally a rho kinase inhibitor, a Smoothened antagonist, a FGFR2b agonist, a protein kinase C activator, or a BMP type 1 receptor inhibitor for an amount of time sufficient to form an early pancreas progenitor cell; (iv) incubating an early pancreas progenitor cell differentiated from the modified PSC for at least about 3 days and optionally contacting the early pancreas progenitor cell with a rho kinase inhibitor, a TGF-/Activin agonist, a Smoothened antagonist, an FGFR2b agonist, or a RAR agonist for an amount of time sufficient to form a pancreatic progenitor cell; (v) contacting a pancreatic progenitor cell differentiated from the modified PSC with an Alk5 inhibitor, a gamma secretase inhibitor, a Smoothened antagonist, an Erbb1 (EGFR) or Erbb4 agonist, and/or a RAR agonist for an amount of time sufficient to form an endoderm cell, wherein during at least a portion of the contacting in (v) depolymerizing the actin cytoskeleton at a time and for an amount of time sufficient to increase differentiation efficiency; and/or (vi) incubating an endoderm cell for an amount of time in serum-free media sufficient to form a beta cell, and within about 24 hours of incubation resizing the beta cells that formed into beta cell clusters.

In some of any of the provided embodiments, the culturing the modified PSC under conditions sufficient for differentiation of the modified PSC into the modified SC-beta cell comprises (i) contacting the modified PSC with a TGF/Activin agonist or a glycogen synthase kinase 3 (GSK) inhibitor or WNT agonist for an amount of time sufficient to form a definitive endoderm cell; (ii) contacting the definitive endoderm cell with a FGFR2b agonist for an amount of time sufficient to form a primitive gut tube cell; (iii) contacting the primitive gut tube cell with an RAR agonist, and optionally a rho kinase inhibitor, a Smoothened antagonist, a FGFR2b agonist, a protein kinase C activator, or a BMP type 1 receptor inhibitor for an amount of time sufficient to form an early pancreas progenitor cell; (iv) incubating the early pancreas progenitor cell for at least about 3 days and optionally contacting the early pancreas progenitor cell with a rho kinase inhibitor, a TGF-/Activin agonist, a Smoothened antagonist, an FGFR2b agonist, or a RAR agonist for an amount of time sufficient to form a pancreatic progenitor cell; (v) contacting the pancreatic progenitor cell with an Alk5 inhibitor, a gamma secretase inhibitor, SANT 1, Erbb1 (EGFR) or Erbb4 agonist, or a RAR agonist for an amount of time sufficient to form an endoderm cell, wherein during at least a portion of the contacting in (v) depolymerizing the actin cytoskeleton at a time and for an amount of time sufficient to increase differentiation efficiency; and (vi) incubating the endoderm cell for an amount of time in serum-free media sufficient to form a beta cell, and within about 24 hours of incubation resizing the beta cells that formed into beta cell clusters.

In some of any of the provided embodiments, depolymerizing the actin cytoskeleton comprises plating cells on a stiff or soft substrate or introducing a cytoskeletal-modulating agent to cells. In some of any of the provided embodiments, the cytoskeletal-modulating agent comprises latrunculin A, latrunculin B, nocodazole, cytochalasin D, jasplakinolide, blebbistatin, y-27632, y-15, gdc-0994, or an integrin modulating agent. In some of any of the provided embodiments, the cytoskeletal-modulating agent is latrunculin A. In some of any of the provided embodiments, depolymerizing the actin cytoskeleton is initiated at the start of the contacting in (v). In some of any of the provided embodiments, depolymerizing the actin cytoskeleton comprises adding latrunculin A at the start of the contacting for at least at or about the first 24 hours. In some of any of the provided embodiments, resizing the beta cell clusters comprises breaking apart clusters and reaggregating.

In some of any of the provided embodiments, the TGF/Activin agonist is Activin A. In some of any of the provided embodiments, the glycogen synthase kinase 3 (GSK) inhibitor or the WNT agonist is CHIR. In some of any of the provided embodiments, the FGFR2b agonist is KGF. In some of any of the provided embodiments, the Smoothened antagonist is SANT-1. In some of any of the provided embodiments, the RAR agonist is retinoic acid (RA). In some of any of the provided embodiments, the protein kinase C activator is TPPB. In some of any of the provided embodiments, the BMP type 1 receptor inhibitor is LDN. In some of any of the provided embodiments, the rho kinase inhibitor is Y27632. In some of any of the provided embodiments, the Alk5 inhibitor is Alk5i. In some of any of the provided embodiments, the Erbb4 agonist is betacellulin. In some of any of the provided embodiments, the thyroid hormone is T3. In some of any of the provided embodiments, the gamma secretase inhibitor is XXI.

In some of any of the provided embodiments, the PSC is an embryonic stem cell. In some of any of the provided embodiments, the PSC is an induced PSC (iPSC). In some embodiments, the iPSC is a patient-derived iPSC.

In some of any of the provided embodiments, the modified PSC expresses each of the one or more tolerogenic factors at a first level that is greater than at or about 5-fold over a second level expressed by the control or wild-type PSC. In some of any of the provided embodiments, each of the one or more tolerogenic factors is expressed at a first level that is greater than at or about 10-fold, greater than at or about 20-fold, greater than at or about 30-fold, greater than at or about 40-fold, greater than at or about 50-fold, greater than at or about 60-fold, or greater than at or about 70-fold over a second level expressed by the control or wild-type PSC.

In some of any of the provided embodiments, each of the one or more tolerogenic factors is expressed by the modified PSC at greater than at or about 20,000 molecules per cell. In some of any of the provided embodiments, each of the one or more tolerogenic factors is expressed by the modified PSC at greater than at or about 30,000 molecules per cell, greater than at or about 50,000 molecules per cell, greater than at or about 100,000 molecules per cell, greater than at or about 200,000 molecules per cell, greater than at or about 300,000 molecules per cell, greater than at or about 400,000 molecules per cell, greater than at or about 500,000 molecules per cell, or greater than at or about 600,000 molecules per cell.

In some of any of the provided embodiments, the one or more tolerogenic factors is or comprises CD47 and the modified PSC expresses CD47 at a first level that is greater than at or about 5-fold over a second level expressed by the control or wild-type PSC. In some of any of the provided embodiments, CD47 is expressed at a first level that is greater than at or about 10-fold, greater than at or about 20-fold, greater than at or about 30-fold, greater than at or about 40-fold, greater than at or about 50-fold, greater than at or about 60-fold, or greater than at or about 70-fold over a second level expressed by the control or wild-type PSC.

In some of any of the provided embodiments, the one or more tolerogenic factor is CD47 and CD47 is expressed by the modified PSC at greater than at or about 20,000 molecules per cell. In some of any of the provided embodiments, CD47 is expressed by the modified PSC at greater than at or about 30,000 molecules per cell, greater than at or about 50,000 molecules per cell, greater than at or about 100,000 molecules per cell, greater than at or about 200,000 molecules per cell, greater than at or about 300,000 molecules per cell, greater than at or about 400,000 molecules per cell, greater than at or about 500,000 molecules per cell, or greater than at or about 600,000 molecules per cell.

In some of any of the provided embodiments, the modified SC-beta cell comprises the modifications of the modified PSC.

In some of any embodiments of the provided methods, the modified SC-beta cell comprises modifications that (1) reduce expression of one or more MHC class I molecules and/or one or more MHC class II molecules, relative to a control or wild-type beta cell; and (2) increase expression of one or more tolerogenic factors, relative to the control or wild-type beta cell. In some of any of the provided embodiments, the control or wild-type SC-beta cell is an unmodified SC-beta cell differentiated from an unmodified PSC not comprising modifications that reduce expression of the one or more MHC class I molecules and/or the one or more MHC class II molecules or that increase expression of the one or more tolerogenic factors. In some of any of the provided embodiments, the control or wild-type beta cell is a wild-type primary beta cell.

In some of any of the provided embodiments, expression of the one or more MHC class I molecules and the one or more MHC class II molecules is reduced in the modified SC-beta cell.

In some of any of the provided embodiments, the modifications in (1) reduce protein expression of the one or more MHC class I molecules in the modified SC-beta cell. In some of any of the provided embodiments, the modifications in (1) reduce cell surface expression of the one or more MHC class I molecules in the modified SC-beta cell. In some of any of the provided embodiments, the modifications in (1) reduce a function of the one or more MHC class I molecules in the modified SC-beta cell. In some embodiments, the function is antigen presentation.

In some of any of the provided embodiments, the one or more MHC class I molecules is one or more human leukocyte antigen (HLA) class I molecules. In some of any of the provided embodiments, the one or more MHC HLA class I molecules is selected from the group consisting of HLA-A, HLA-B, and HLA-C.

In some of any of the provided embodiments, the modification that reduces expression of the one or more MHC class I molecules in the modified SC-beta cell comprises reduced expression of B2M. In some of any of the provided embodiments, the modification that reduces expression of the one or more MHC class I molecules in the modified SC-beta cell reduces mRNA expression of the B2M gene. In some of any of the provided embodiments, the modification that reduces expression of the one or more MHC class I molecules in the modified SC-beta cell reduces protein expression of B2M.

In some of any embodiments, the modification that reduces expression of the one or more MHC class I molecules in the modified SC-beta cell comprises inactivation or disruption of one allele of the B2M gene. In some of any embodiments, the modification that reduces expression of the one or more MHC class I molecules in the modified SC-beta cell comprises inactivation or disruption of both alleles of the B2M gene. In some of any embodiments, the modification that reduces expression of the one or more MHC class I molecules in the modified SC-beta cell comprises inactivation or disruption of all B2M coding alleles in the cell. In some of any embodiments, the inactivation or disruption comprises an indel in the B2M gene. In some of any embodiments, the inactivation or disruption comprises a frameshift mutation or a deletion of a contiguous stretch of genomic DNA of the B2M gene.

In some of any embodiments, the modifications in (1) reduce protein expression of the one or more MHC class II molecules in the modified SC-beta cell. In some of any embodiments, the modifications in (1) reduce cell surface expression of the one or more MHC class II molecules in the modified SC-beta cell. In some of any embodiments, the modifications in (1) reduce a function of the one or more MHC class II molecules in the modified SC-beta cell. In some embodiments, the function is antigen presentation.

In some of any embodiments, the one or more MHC class II molecules is one or more human leukocyte antigen (HLA) class II molecules. In some embodiments, the one or more MHC class II molecules is selected from the group consisting of HLA-DP, HLA-DQ, and/or HLA-DR.

In some of any embodiments, the modification that reduces expression of the one or more MHC class II molecules in the modified SC-beta cell comprises reduced expression of CIITA. In some of any embodiments, the modification that reduces expression of the one or more MHC class II molecules in the modified SC-beta cell reduces mRNA expression of the CIITA gene. In some of any embodiments, the modification that reduces expression of the one or more MHC class II molecules in the modified SC-beta cell reduces protein expression of CIITA. In some of any embodiments, the modification that reduces expression of the one or more MHC class II molecules comprises inactivation or disruption of one allele of the CIITA gene. In some of any embodiments, the modification that reduces expression of the one or more MHC class II molecules comprises inactivation or disruption of both alleles of the CIITA gene. In some of any embodiments, the modification that reduces expression of the one or more MHC class II molecules comprises inactivation or disruption of all CIITA coding alleles in the cell. In some of any embodiments, the inactivation or disruption comprises an indel in the CIITA gene. In some of any embodiments, the inactivation or disruption is a frameshift mutation or a deletion of a contiguous stretch of genomic DNA of the CIITA gene.

In some of any of the provided embodiments, expression of all MHC class I molecules and all MHC class II molecules is reduced in the modified SC-beta cell. In some embodiments, expression of HLA-A, HLA-B, HLA-C, HLA-DP, HLA-DQ, and HLA-DR are reduced in the modified SC-beta cell.

In some of any embodiments, the modified SC-beta cell comprises a modification that reduces expression of CD142. In some of any embodiments, the modification reduces mRNA expression of the CD142 gene. In some of any embodiments, the modification reduces protein expression of CD142. In some of any embodiments, the modification comprises inactivation or disruption of one allele of the CD142 gene. In some of any embodiments, the modification comprises inactivation or disruption of both alleles of the CD142 gene. In some of any embodiments, the modification comprises inactivation or disruption of all CD142 coding alleles in the cell. In some of any embodiments, the inactivation or disruption comprises an indel in the CD142 gene. In some of any embodiments, the inactivation or disruption is a frameshift mutation or a deletion of a contiguous stretch of genomic DNA of the CD142 gene.

In some of any embodiments, the modification to increase expression of the one or more tolerogenic factors in the modified SC-beta cell comprises an exogenous polynucleotide encoding the one or more tolerogenic factors. In some of any embodiments, the exogenous polynucleotide encoding the one or more tolerogenic factors is integrated into the genome of the modified SC-beta cell. In some of any embodiments, the exogenous polynucleotide encoding the one or more tolerogenic factors is integrated into a non-target locus in the genome of the modified SC-beta cell. In some of any embodiments, the exogenous polynucleotide encoding the one or more tolerogenic factors is integrated into a target genomic locus of the modified SC-beta cell. In some embodiments, the tolerogenic factor is CD47. In some of any embodiments, the modified SC-beta cell further comprises a modification for expression of an exogenous suicide gene in the modified SC-beta cell.

In some embodiments, the modified SC-beta cell generated by the provided methods comprises knock out of the B2M gene, knock out of the CIITA gene, and an exogenous polynucleotide encoding exogenous CD47 protein, relative to a control or wild-type beta cell. In some embodiments, the modified SC-beta cell has the phenotype B2M^{indel/indel}; CIITA^{indel/indel}; CD47tg.

In some embodiments, the modified SC-beta cell generated by the provided methods comprises knock out of the B2M gene, knock out of the CIITA gene, an exogenous polynucleotide encoding CD47 protein, and an exogenous polynucleotide encoding a suicide gene, relative to a control or wild-type beta cell. In some embodiments, the modified SC-beta cell has the phenotype B2M^{indel/indel}; CIITA^{indel/indel}; CD47tg; suicide genetg.

In some of any embodiments, the exogenous polynucleotide encoding CD47 is integrated into a non-target locus in the genome of the modified SC-beta cell. In some of any embodiments, the exogenous polynucleotide encoding CD47 is integrated into a target genomic locus of the modified SC-beta cell.

In some of any embodiments, the exogenous suicide gene is selected from the group consisting of cytosine deaminase (CyD), herpesvirus thymidine kinase (HSV-Tk), an inducible caspase (iCaspase9), and rapamycin-activated caspase 9 (rapaCasp9). In some of any embodiments, the suicide gene and the one or more tolerogenic factors are expressed from a bicistronic cassette integrated into the genome of the modified SC-beta cell. In some of any embodiments, the one or more tolerogenic factors is CD47 and the suicide gene and CD47 are expressed from a bicistronic cassette integrated into the genome of the modified SC-beta cell. In some of any embodiments, the bicistronic cassette is integrated at a non-target locus in the genome of the modified SC-beta cell. In some of any embodiments, the bicistronic cassette is integrated into a target genomic locus of the cell. In some of any embodiments, the target genomic locus is a safe harbor locus, a B2M gene locus, a CIITA gene locus, or a CD142 gene locus. In some of any embodiments, the safe harbor locus is selected from the group consisting of: a CCR5 gene locus, a CXCR4 gene locus, a PPP1R12C (also known as AAVS1) gene, an albumin gene locus, a SHS231 locus, a CLYBL gene locus, and a ROSA26 gene locus.

In some of any embodiments, the methods generate a modified SC-beta cell that comprises a modification that increases expression of one or more complement inhibitors selected from the group consisting of CD46, CD59, CD55 and CD35, relative to the control or wild-type beta cell. In some of any embodiments, the modification to increase expression of the one or more complement inhibitors in the modified SC-beta cell comprises at least one exogenous polynucleotide encoding the one or more complement inhibitors selected from the group consisting of an exogenous polynucleotide encoding CD46, an exogenous polynucleotide encoding CD59, an exogenous polynucleotide encoding CD55, and an exogenous polynucleotide encoding CD35. In some of any embodiments, the one or more complement inhibitors is CD46 and CD59. In some of any embodiments, the one or more complement inhibitor is CD46, CD59 and CD55.

In some of any embodiments, the reduced expression comprises reduced surface expression. In some of any embodiments, the increased expression comprises increased surface expression. In some of any embodiments, the level of the reduced expression of (1) and the increased expression of (2) by the modified SC-beta cell is retained or is similar compared to the modified PSC.

In some of any embodiments, the methods generate a modified SC-beta cell that expresses the one or more tolerogenic factors at a first level that is greater than at or about 5-fold over a second level expressed by the control or wild-type beta cell. In some of any embodiments the control or wild-type beta cell is differentiated from an unmodified PSC not comprising modifications that reduce expression of the one or more MHC class I molecules and/or the one or more MHC class II molecules and that increase expression of the one or more tolerogenic factors. In some of any embodiments, the modified SC-beta cell expresses each of the one or more tolerogenic factors at a first level that is greater than at or about 5-fold over a second level expressed by the control or wild-type beta cell. In some of any embodiments, each of the one or more tolerogenic factors is expressed at a first level that is greater than at or about 10-fold, greater than at or about 20-fold, greater than at or about 30-fold, greater than at or about 40-fold, greater than at or about 50-fold, greater than at or about 60-fold, or greater than at or about 70-fold over a second level expressed by the control or wild-type beta cell.

In some of any embodiments, each of the one or more tolerogenic factors is expressed by the modified SC-beta cell at greater than at or about 20,000 molecules per cell. In some of any embodiments, each of the one or more tolerogenic factors is expressed by the modified SC-beta cell at greater than at or about 30,000 molecules per cell, greater than at or about 50,000 molecules per cell, greater than at or about 100,000 molecules per cell, greater than at or about 200,000 molecules per cell, greater than at or about 300,000 molecules per cell, greater than at or about 400,000 molecules per cell, greater than at or about 500,000 molecules per cell, or greater than at or about 600,000 molecules per cell.

In some of any embodiments, the one or more tolerogenic factors is or comprises CD47 and the modified SC-beta cell expresses CD47 at a first level that is greater than at or about 5-fold over a second level expressed by the control or wild-type beta cell. In some of any embodiments, the control or wild-type beta cell is differentiated from an unmodified PSC not comprising modifications that reduce expression of the one or more MHC class I molecules and/or the one or more MHC class II molecules and that increase expression of the one or more tolerogenic factors. In some of any embodiments, the modified SC-beta cell expresses CD47 at a first level that is greater than at or about 5-fold over a second level expressed by the control or wild-type beta cell. In some of any embodiments, CD47 is expressed at a first level that is greater than at or about 10-fold, greater than at or about 20-fold, greater than at or about 30-fold, greater than at or about 40-fold, greater than at or about 50-fold, greater than at or about 60-fold, or greater than at or about 70-fold over a second level expressed by the control or wild-type beta cell. In some of any embodiments, CD47 is expressed by the modified SC-beta cell at greater than at or about 20,000 molecules per cell. In some of any embodiments, CD47 is expressed by the modified SC-beta cell at greater than at or about 30,000 molecules per cell, greater than at or about 50,000 molecules per cell, greater than at or about 100,000 molecules per cell, greater than at or about 200,000 molecules per cell, greater than at or about 300,000 molecules per cell, greater than at or about 400,000 molecules per cell, greater than at or about 500,000 molecules per cell, or greater than at or about 600,000 molecules per cell.

In some of any embodiments, the modified SC-beta cell expresses at least one beta cell marker. In some of any embodiments, the at least one beta cell marker is selected from the group consisting of INS, CHGA, NKX2-2, PDX1, NKX6-1, MAFB, GCK and GLUT1. In some of any embodiments, the modified SC-beta cell exhibits one or more functions of a wild-type or control beta cell. In some embodiments, the one or more functions is selected from the group consisting of in vitro glucose-stimulated insulin secretion (GSIS), glucose metabolism, maintaining fasting blood glucose levels, secreting insulin in response to glucose injections in vivo, and clearing glucose after a glucose injection in vivo.

In some of any embodiments, the modified SC-beta cell is capable of glucose-stimulated insulin secretion (GSIS). In some embodiments, the insulin secretion is in a perfusion GSIS assay. In some of any embodiments, the GSIS is dynamic GSIS comprising first and second phase dynamic insulin secretion. In some of any embodiments, the GSIS is static GSIS. In some embodiments, the static incubation index is greater than at or about 1, greater than at or about 2, greater than at or about 5, greater than at or about 10 or greater than at or about 20. In some of any embodiments, the level of insulin secretion by the modified SC-beta cells is at least 20% of that observed for primary beta islets, such as observed for cadaveric islets. In some of any embodiments, the level of insulin secretion by the modified SC-beta cells is at least 25%, at least 30%, at least 40%, at least 50%, at least 60%, at least 70% of that observed for primary beta islets, such as observed for cadaveric islets. In some of any embodiments, the total insulin content of the modified SC-beta cell is greater than at or about 500 μIU Insulin per 5000 cells, greater than at or about 1000 μIU Insulin per 5000 cells, greater than at or about 2000 μIU Insulin per 5000 cells, greater than at or about 3000 μIU Insulin per 5000 cells or greater than at or about 4000 μIU Insulin per 5000 cells. In some of any embodiments, the proinsulin to insulin ratio of the modified SC-beta cell is between at or about 0.02 and at or about 0.1, optionally at or about 0.03, 0.04, 0.05, 0.06, 0.07, 0.08, 0.09 and any value between any of the foregoing.

In some of any embodiments, the modified SC-beta cells exhibit functionality for 1 or more days following transplantation into a subject. In some of any embodiments, the modified SC-beta cells exhibit functionality for more than 1 week following transplantation into a subject. In some of any embodiments, the functionality is selected from the group consisting of maintaining fasting blood glucose levels, secreting insulin in response to glucose injections in vivo, and clearing glucose after a glucose injection in vivo.

Provided herein is a composition comprising a population of modified SC-beta cells produced by any of the provided methods.

Provided herein is a modified stem-cell derived beta cell (SC-beta cell) that has been differentiated in vitro from a pluripotent stem cell (PSC), wherein the modified SC-beta cell has (1) reduced expression of one or more major histocompatibility complex (MHC) class I molecules and/or one or more MHC class II molecules, relative to a control or wild-type beta cell; and (2) increased expression of a tolerogenic factor, relative to the control or wild-type beta cell, and wherein the modified SC-beta cell exhibits glucose-stimulated insulin secretion (GSIS).

In some of any embodiments, the tolerogenic factor is expressed at a first level that is greater than at or about 10-fold, greater than at or about 20-fold, greater than at or about 30-fold, greater than at or about 40-fold, greater than at or about 50-fold, greater than at or about 60-fold, or greater than at or about 70-fold over a second level expressed by the control or wild-type beta cell.

Provided herein is a modified stem-cell derived beta cell (SC-beta cell) comprising one or more modifications that: (a) inactivate or disrupt one or more alleles of: (i) one or more major histocompatibility complex (MHC) class I molecules or one or more molecules that regulate expression of the one or more MHC class I molecules, and/or (ii) one or more MHC class II molecules or one or more molecules that regulate expression of the one or more MHC class II molecules, and/or (b) increase expression of one or more tolerogenic factors, wherein the increased expression is relative to a control or wild-type beta cell that does not comprise the modifications. In some embodiments, the modified SC-beta cell exhibits glucose-stimulated insulin secretion (GSIS).

Provided herein is a modified stem-cell derived beta cell (SC-beta cell) that has been differentiated in vitro from a pluripotent stem cell (PSC), wherein the modified SC-beta cell has modifications that (a) inactivate or disrupt one or more alleles of: (i) one or more major histocompatibility complex (MHC) class I molecules or one or more molecules that regulate expression of the one or more MHC class I molecules, and/or (ii) one or more MHC class II molecules or one or more molecules that regulate expression of the one or more MHC class II molecules; and (b) increase expression of one or more tolerogenic factors, relative to a control or wild-type beta cell, and wherein the modified SC-beta cell exhibits glucose-stimulated insulin secretion (GSIS).

Provided herein is a modified stem cell-derived beta cell (SC-beta cell) that has been differentiated in vitro from a pluripotent stem cell (PSC), wherein the modified SC-beta (1) does not express one or more major histocompatibility complex (MHC) class I molecules and/or one or more MHC class II molecules and (2) overexpresses a tolerogenic factor at a level of greater than at or about 5-fold compared to background, and wherein the modified SC-beta cell exhibits glucose-stimulated insulin secretion (GSIS). In some of any embodiments, the expression of the tolerogenic factor is by flow cytometry with an antibody directed against the tolerogenic factor and the background is determined by flow cytometry staining with an isotype control of the antibody. In some of any embodiments, the tolerogenic factor is expressed at a level that is greater than at or about 10-fold, greater than at or about 20-fold, greater than at or about 30-fold, greater than at or about 40-fold, greater than at or about 50-fold, greater than at or about 60-fold, or greater than at or about 70-fold compared to background expression.

Provided herein is a modified stem cell-derived beta cell (SC-beta cell) that has been differentiated in vitro from a pluripotent stem cell (PSC), wherein the modified SC-beta cell (1) does not express one or more major histocompatibility complex (MHC) class I molecules or one or more MHC class II molecules and (2) overexpresses a tolerogenic factor at a first level of greater than at or about 5-fold over a second level expressed by an unmodified cell, wherein the unmodified cell is an unmodified PSC that does not comprise modifications to reduce the one or more MHC class I molecules and/or the one or more MHC class II molecules and to overexpress the tolerogenic factor or is an unmodified SC-beta cell differentiated from such unmodified PSC; and wherein the modified SC-beta cell exhibits glucose-stimulated insulin secretion (GSIS).

In some of any embodiments, the tolerogenic factor is expressed by the modified SC-beta cell at greater than at or about 20,000 molecules per cell.

Provided herein is a modified stem cell-derived beta cell (SC-beta cell) that has been differentiated in vitro from a pluripotent stem cell (PSC), wherein the modified SC-beta cell (1) does not express one or more major histocompatibility complex (MHC) class I molecules or one or more MHC class II molecules and (2) overexpresses a tolerogenic factor, wherein the tolerogenic factor is expressed at greater than at or about 20,000 molecules per cell, and wherein the modified beta cell exhibits glucose-stimulated insulin secretion (GSIS).

In some of any embodiments, the tolerogenic factor is expressed by the modified SC-beta at greater than at or about 30,000 molecules per cell, greater than at or about 50,000 molecules per cell, greater than at or about 100,000 molecules per cell, greater than at or about 200,000 molecules per cell, greater than at or about 300,000 molecules per cell, greater than at or about 400,000 molecules per cell, greater than at or about 500,000 molecules per cell, or greater than at or about 600,000 molecules per cell.

In some of any embodiments, the modified SC-beta cell is differentiated from a PSC in which the PSC is a modified PSC comprising modifications that (a) reduce expression of one or more MHC class I molecules and/or one or more MHC class II molecules, relative to a control or wild-type PSC; and (b) increase expression of a tolerogenic factor, relative to the control or wild-type PSC. In some of any embodiments, the control or wild-type PSC is an unmodified PSC that does not comprise the modifications. In some of any embodiments, the modified SC-beta cell expresses the tolerogenic factor at a first level that is greater than at or about 5-fold over a second level expressed by the unmodified PSC or the unmodified SC-beta cell differentiated from the unmodified PSC. In some of any embodiments, the tolerogenic factor is expressed at a first level that is greater than at or about 10-fold, greater than at or about 20-fold, greater than at or about 30-fold, greater than at or about 40-fold, greater than at or about 50-fold, greater than at or about 60-fold, or greater than at or about 70-fold over a second level expressed by the unmodified PSC or an unmodified SC-beta differentiated from the unmodified PSC.

Provided herein is a modified stem-cell derived beta cell (SC-beta cell) that has been differentiated in vitro from a modified pluripotent stem cell (PSC), wherein the modified PSC comprises modifications that (a) reduce expression of one or more major histocompatibility complex (MHC) class I molecules or one or more MHC class II molecules, relative to a control or wild-type PSC; and (b) increase expression of a tolerogenic factor, relative to the control or wild-type PSC, and wherein the modified SC-beta cell exhibits glucose-stimulated insulin secretion (GSIS).

In some of any embodiments, the control or wild-type PSC is an unmodified PSC that does not comprise the modifications. In some of any embodiments, the modified PSC expresses the tolerogenic factor at a first level that is greater than at or about 5-fold over a second level expressed by the unmodified PSC that does not comprise the modifications. In some embodiments, the tolerogenic factor is expressed at a first level that is greater than at or about 10-fold, greater than at or about 20-fold, greater than at or about 30-fold, greater than at or about 40-fold, greater than at or about 50-fold, greater than at or about 60-fold, or greater than at or about 70-fold over the second level expressed by the unmodified PSC.

In some of any embodiments, the modified SC-beta cell comprises modifications that (a) reduce expression of the one or more MHC class I molecules and/or or the one or more MHC class II molecule, relative to the unmodified PSC or an unmodified SC-beta differentiated from the unmodified PSC; and (b) increase expression of a tolerogenic factor, compared to the unmodified PSC or the unmodified SC-beta differentiated from the unmodified PSC. In some of any embodiments, the modified SC-beta expresses the tolerogenic factor at a first level that is greater than at or about 5-fold over a second level expressed by the unmodified PSC or the unmodified SC-beta cell differentiated from an unmodified PSC. In some of any embodiments, the tolerogenic factor is expressed at a first level that is greater than at or about 10-fold, greater than at or about 20-fold, greater than at or about 30-fold, greater than at or about 40-fold, greater than at or about 50-fold, greater than at or about 60-fold, or greater than at or about 70-fold over a second level expressed by the unmodified PSC or the unmodified SC-beta cell differentiated from an unmodified PSC. In some of any embodiments, the tolerogenic factor is expressed by the modified PSC at greater than at or about 20,000 molecules per cell.

Provided herein is a modified stem cell-derived beta cell (SC-beta cell) that has been differentiated in vitro from a modified pluripotent stem cell (PSC), wherein the modified PSC comprises modifications such that the modified PSC (a) does not express one or more major histocompatibility complex (MHC) class I molecules and/or or one or more MHC class II molecule; and (b) expresses a tolerogenic factor at greater than at or about 20,000 molecules per cell, and wherein the modified SC-beta cell exhibits glucose-stimulated insulin secretion (GSIS).

In some of any embodiments, the tolerogenic factor is expressed by the modified PSC at greater than at or about 30,000 molecules per cell, greater than at or about 50,000 molecules per cell, greater than at or about 100,000 molecules per cell, greater than at or about 200,000 molecules per cell, greater than at or about 300,000 molecules per cell, greater than at or about 400,000 molecules per cell, greater than at or about 500,000 molecules per cell, or greater than at or about 600,000 molecules per cell. In some of any embodiments, the modified SC-beta cell does not express the one or more MHC class I molecule or the one or more MHC class II molecule and expresses the tolerogenic factor at greater than at or about 20,000 molecules per cell. In some of any embodiments, the tolerogenic factor is expressed by the modified SC-beta cell at greater than at or about 30,000 molecules per cell, greater than at or about 50,000 molecules per cell, greater than at or about 100,000 molecules per cell, greater than at or about 200,000 molecules per cell, greater than at or about 300,000 molecules per cell, greater than at or about 400,000 molecules per cell, greater than at or about 500,000 molecules per cell, or greater than at or about 600,000 molecules per cell.

In some of any embodiments, the tolerogenic factor is selected from the group consisting of CD47, CD27, CD200, HLA-C, HLA-E, HLA-E heavy chain, HLA-G, PD-L1, IDO1, CTLA4-Ig, Cl-Inhibitor, IL-10, IL-35, FASL, CCL21, MFGE8, and SERPINB9, and any combination thereof. In some of any embodiments, the tolerogenic factor comprises CD47. In some of any embodiments, the tolerogenic factor comprises PD-L1. In some of any embodiments, the tolerogenic factor comprises HLA-E. In some of any embodiments, the tolerogenic factor comprises HLA-G.

In some of any embodiments, the modified SC-beta cell is differentiated from a modified PSC and expression of the one or more MHC class I molecules and the one or more MHC class II molecules is reduced in the modified PSC.

In some of any embodiments, the modifications in (a) reduce protein expression of the one or more MHC class I molecules. In some of any embodiments, the modifications in (a) reduce cell surface expression of one or more MHC class I molecules. In some of any embodiments, the one or more modifications in (a) reduce cell surface trafficking of the one or more MHC class I molecules. In some of any embodiments, the modifications in (a) reduce a function of one or more MHC class I molecules. In some embodiments, the function is antigen presentation.

In some of any embodiments, the modified SC-beta cell is differentiated from a modified PSC in which the modification that reduces expression of the one or more MHC class I reduces expression of B2M. In some of any embodiments, the modification that reduces expression of the one or more MHC class I molecules reduces mRNA expression of the B2M gene. In some of any embodiments, the modification that reduces expression of the one or more MHC class I molecules reduces protein expression of B2M. In some of any embodiments, the modification that reduces expression of the one or more MHC class I molecules in the modified PSC comprises inactivation or disruption of one allele of the B2M gene. In some of any embodiments, the modification that reduces expression of the one or more MHC class I molecules in the modified PSC comprises inactivation or disruption of both alleles of the B2M gene. In some of any embodiments, the modification that reduces expression of the one or more MHC class I molecules in the modified PSC comprises inactivation or disruption of all B2M coding alleles in the cell. In some of any embodiments, the inactivation or disruption comprises an indel in the B2M gene. In some of any embodiments, the inactivation or disruption is a frameshift mutation or a deletion of a contiguous stretch of genomic DNA of the B2M gene.

In some of any embodiments, the modifications in (a) reduce protein expression of the one or more MHC class II molecules. In some of any embodiments, the modifications in (a) reduce cell surface expression of the one or more MHC class II molecules. In some of any embodiments, the one or more modifications in (a) reduce cell surface trafficking of the one or more MHC class II molecules. In some of any embodiments, the modifications in (a) reduce a function of the one or more MHC class II molecules. In some embodiments, the function is antigen presentation.

In some of any embodiments, the modified SC-beta cell is differentiated from a modified PSC in which the modification that reduces expression of the one or more MHC class II comprises reduced expression of CIITA. In some of any embodiments, the modification that reduces expression of the one or more MHC class II molecules in the modified PSC reduces mRNA expression of the CIITA gene. In some of any embodiments, the modification that reduces expression of the one or more MHC class II molecules in the modified PSC reduces protein expression of CIITA. In some of any embodiments, the modification that reduces expression of the one or more MHC class II molecules in the modified PSC comprises inactivation or disruption of one allele of the CIITA gene. In some of any embodiments, the modification that reduces expression of the one or more MHC class II molecules in the modified PSC comprises inactivation or disruption of both alleles of the CIITA gene. In some of any embodiments, the modification that reduces expression of the one or more MHC class II molecules in the modified PSC comprises inactivation or disruption of all CIITA coding alleles in the cell. In some of any embodiments, the inactivation or disruption comprises an indel in the CIITA gene. In some of any embodiments, the inactivation or disruption is a frameshift mutation or a deletion of a contiguous stretch of genomic DNA of the CIITA gene.

In some of any of the provided embodiments, expression of all MHC class I molecules and all MHC class II molecules is reduced in the modified PSC. In some embodiments, expression of HLA-A, HLA-B, HLA-C, HLA-DP, HLA-DQ, and HLA-DR are reduced in the modified PSC.

In some of any embodiments, the modified SC-beta cell is differentiated from a modified PSC and the modified PSC comprises a modification that reduces expression of CD142. In some of any embodiments, the modification reduces mRNA expression of the CD142 gene. In some of any embodiments, the modification reduces protein expression of CD142. In some of any embodiments, the modification that reduces expression of CD142 in the modified PSC comprises inactivation or disruption of one allele of the CD142 gene. In some of any embodiments, the modification that reduces expression of CD142 in the modified PSC comprises inactivation or disruption of both alleles of the CD142 gene. In some of any embodiments, the modification that reduces expression of CD142 in the modified PSC comprises inactivation or disruption of all CD142 coding alleles in the cell. In some of any embodiments, the inactivation or disruption comprises an indel in the CD142 gene. In some of any embodiments, the inactivation or disruption is a frameshift mutation or a deletion of a contiguous stretch of genomic DNA of the CD142 gene.

In some of any embodiments, the modification to increase expression of the tolerogenic factor in the modified PSC comprises an exogenous polynucleotide encoding the tolerogenic factor. In some of any embodiments, the exogenous polynucleotide encoding the tolerogenic factor is integrated into the genome of the modified PSC. In some of any embodiments, the exogenous polynucleotide is integrated by non-targeted insertion into the genome of the modified PSC. In some of any embodiments, the exogenous polynucleotide is integrated by targeted insertion into a target genomic locus of the modified PSC.

In some of any embodiments, the modified SC-beta cell is differentiated from a modified PSC in which the modified PSC further comprises an exogenous polynucleotide encoding a suicide gene. In some of any embodiments, the suicide gene is selected from the group consisting of cytosine deaminase (CyD), herpesvirus thymidine kinase (HSV-Tk), an inducible caspase (iCaspase9), and rapamycin-activated caspase 9 (rapaCasp9).

In some of any embodiments, the suicide gene and the tolerogenic factor are expressed from a bicistronic cassette integrated into the genome of the modified PSC. In some embodiments, the tolerogenic factor is CD47 and the suicide gene and CD47 are expressed from a bicistronic cassette integrated into the genome of the modified PSC. In some of any embodiments, the bicistronic cassette is integrated by non-targeted insertion into the genome of the modified PSC. In some of any embodiments, the bicistronic cassette is integrated by targeted insertion into a target genomic locus of the modified PSC. In some of any embodiments, the target genomic locus is a safe harbor locus, a B2M gene locus, a CIITA gene locus, or a CD142 gene locus. In some of any embodiments, the safe harbor locus is selected from the group consisting of: a CCR5 gene locus, a CXCR4 gene locus, a PPP1R12C (also known as AAVS1) gene, an albumin gene locus, a SHS231 locus, a CLYBL gene locus, and a ROSA26 gene locus.

In some of any embodiments, the modified SC-beta cell is differentiated from a modified PSC and the modified PSC comprises a modification that increases expression of one or more complement inhibitors selected from the group consisting of CD46, CD59, CD55 and CD35, relative to the control or wild-type PSC. In some of any embodiments, the modification to increase expression of one or more complement inhibitors comprises at least one exogenous polynucleotide encoding one or more complement inhibitors selected from the group consisting of an exogenous polynucleotide encoding CD46, an exogenous polynucleotide encoding CD59, an exogenous polynucleotide encoding CD55, and an exogenous polynucleotide encoding CD35. In some of any embodiments, the one or more complement inhibitors is CD46 and CD59. In some of any embodiments, the one or more complement inhibitor is CD46, CD59 and CD55. In some of any embodiments, the at least one exogenous polynucleotide is integrated by non-targeted insertion into the genome of the modified PSC. In some of any embodiments, the at least one exogenous polynucleotide is integrated by targeted insertion into a target genomic locus of the cell. In some of any embodiments, the target genomic locus is a safe harbor locus, a B2M gene locus, a CIITA gene locus, or a CD142 gene locus. In some of any embodiments, the safe harbor locus is selected from the group consisting of: a CCR5 gene locus, a CXCR4 gene locus, a PPP1R12C (also known as AAVS1) gene, an albumin gene locus, a SHS231 locus, a CLYBL gene locus, and a ROSA26 gene locus.

In some of any embodiments, the expression of one or more MHC class I molecules and one or more MHC class II molecules is reduced in the modified SC-beta cell.

In some of any embodiments, the modifications in (1) reduce protein expression of one or MHC class I molecules in the modified SC-beta cell. In some of any embodiments, the modifications in (1) reduce cell surface expression of one or more MHC class I molecules in the modified SC-beta cell. In some of any embodiments, the modifications in (1) reduce a function of MHC class I molecules in the modified SC-beta cell. In some embodiments, the function is antigen presentation. In some of any embodiments, the modification that reduces expression of one or more MHC class I molecules in the modified SC-beta cell comprises reduced expression of B2M. In some of any embodiments, the modification that reduces expression of one or more MHC class I in the modified SC-beta cell reduces mRNA expression of the B2M gene. In some of any embodiments, the modification that reduces expression of one or more MHC class I molecules in the modified SC-beta cell reduces protein expression of B2M. In some of any embodiments, the modification that reduces expression of one or more MHC class I molecules in the modified SC-beta cell comprises inactivation or disruption of one allele of the B2M gene. In some of any embodiments, the modification that reduces expression of one or more MHC class I molecules in the modified SC-beta cell comprises inactivation or disruption of both alleles of the B2M gene. In some of any embodiments, the modification that reduces expression of one or more MHC class I molecules in the modified SC-beta cell comprises inactivation or disruption of all B2M coding alleles in the cell. In some of any embodiments, the inactivation or disruption comprises an indel in the B2M gene. In some of any embodiments, the inactivation or disruption is a frameshift mutation or a deletion of a contiguous stretch of genomic DNA of the B2M gene.

In some of any embodiments, the modifications in (1) reduce protein expression of one or more MHC class II molecules in the modified SC-beta cell. In some of any embodiments, the modifications in (1) reduce cell surface expression of one or more MHC class II molecules in the modified SC-beta cell. In some of any embodiments, the modifications in (1) reduce a function of one or more MHC class II molecules in the SC-beta cell. In some embodiments, the function is antigen presentation. In some of any embodiments, the modification that reduces expression of one or more MHC class II molecules in the modified SC-beta cell comprises reduced expression of CIITA. In some of any embodiments, the modification that reduces expression of one or more MHC class II molecules in the modified SC-beta cell reduces mRNA expression of the CIITA gene. In some of any embodiments, the modification that reduces expression of one or more MHC class II molecules in the modified SC-beta cell reduces protein expression of CIITA. In some of any embodiments, the modification that reduces expression of one or more MHC class II molecules in the modified SC-beta cell comprises inactivation or disruption of one allele of the CIITA gene. In some of any embodiments, the modification that reduces expression of one or more MHC class II molecules in the modified SC-beta cell comprises inactivation or disruption of both alleles of the CIITA gene. In some of any embodiments, the modification that reduces expression of one or more MHC class II molecules in the modified SC-beta cell comprises inactivation or disruption of all CIITA coding alleles in the cell. In some of any embodiments, the inactivation or disruption comprises an indel in the CIITA gene. In some of any embodiments, the inactivation or disruption is a frameshift mutation or a deletion of a contiguous stretch of genomic DNA of the CIITA gene.

In some of any of the provided embodiments, expression of all MHC class I molecules and all MHC class II molecules is reduced in the modified SC-beta cell. In some embodiments, expression of HLA-A, HLA-B, HLA-C, HLA-DP, HLA-DQ, and HLA-DR are reduced in the modified SC-beta cell.

In some of any embodiments, the modified SC-beta cell comprises a modification that reduces expression of CD142, relative to a control or wild-type beta cell. In some of any embodiments, the modification reduces mRNA expression of the CD142 gene. In some of any embodiments, the modification reduces protein expression of CD142. In some of any embodiments, the modifications that reduce expression of CD142 in the modified SC-beta cell comprises inactivation or disruption of one allele of the CD142 gene. In some of any embodiments, the modifications that reduce expression of CD142 in the modified SC-beta cell comprises inactivation or disruption of both alleles of the CD142 gene. In some of any embodiments, the modifications that reduce expression of CD142 in the modified SC-beta cell comprises inactivation or disruption of all CD142 coding alleles in the cell. In some of any embodiments, the inactivation or disruption comprises an indel in the CD142 gene. In some of any embodiments, the inactivation or disruption comprises a frameshift mutation or a deletion of a contiguous stretch of genomic DNA of the CD142 gene.

In some of any embodiments, the modification to increase expression of the tolerogenic factor in the modified SC-beta cell comprises an exogenous polynucleotide encoding the tolerogenic factor. In some of any embodiments, the exogenous polynucleotide encoding the tolerogenic factor is integrated into the genome of the modified SC-beta cell. In some of any embodiments, the exogenous polynucleotide is integrated into a non-target locus in the genome of the modified SC-beta cell. In some of any embodiments, the exogenous polynucleotide is integrated into a target genomic locus of the modified SC-beta cell.

In some of any embodiments, the tolerogenic factor is CD47 and the modification to increase expression of CD47 in the modified SC-beta cell comprises an exogenous polynucleotide encoding CD47. In some of any embodiments, the exogenous polynucleotide encoding CD47 is integrated into the genome of the modified SC-beta cell. In some of any embodiments, the exogenous polynucleotide is integrated into a non-target locus in the genome of the modified SC-beta cell. In some of any embodiments, the exogenous polynucleotide is integrated into a target genomic locus of the modified SC-beta cell.

Also provided herein is a modified stem cell derived beta cell (SC-beta cell) that has been differentiated in vitro from a modified pluripotent stem cell (PSC), wherein the modified SC-beta cell comprises knock out of the B2M gene, knock out of the CIITA gene, and an exogenous polynucleotide encoding exogenous CD47 protein, relative to a control or wild-type beta cell. In some embodiments, the modified SC-beta cell has the phenotype B2M^{indel/indel}; CIITA^{indel/indel}; CD47tg. In some embodiments, the modified SC-beta cell further comprises an exogenous polynucleotide encoding a suicide gene.

Also provided herein is a modified stem cell derived beta cell (SC-beta cell) that has been differentiated in vitro from a modified pluripotent stem cell (PSC), wherein the modified SC-beta cell comprises knock out of the B2M gene, knock out of the CIITA gene, an exogenous polynucleotide encoding CD47 protein, and an exogenous polynucleotide encoding a suicide gene, relative to a control or wild-type beta cell. In some embodiments, the modified SC-beta cell has the phenotype B2M^{indel/indel}; CIITA^{indel/indel}; CD47tg; suicide genetg.

In some of any embodiments, the suicide gene is selected from the group consisting of cytosine deaminase (CyD), herpesvirus thymidine kinase (HSV-Tk), an inducible caspase (iCaspase9), and rapamycin-activated caspase 9 (rapaCasp9). In some of any embodiments, the suicide gene and the tolerogenic factor are expressed from a bicistronic cassette integrated into the genome of the modified SC-beta cell. In some of any embodiments, the tolerogenic factor is CD47 and the suicide gene and CD47 are expressed from a bicistronic cassette integrated into the genome of the modified SC-beta cell. In some of any embodiments, the bicistronic cassette is integrated at a non-target locus in the genome of the modified SC-beta cell. In some of any embodiments, the bicistronic cassette is integrated into a target genomic locus of the cell. In some of any embodiments, the target genomic locus is a safe harbor locus, a B2M gene locus, a CIITA gene locus, or a CD142 gene locus. In some of any embodiments, the safe harbor locus is selected from the group consisting of: a CCR5 gene locus, a CXCR4 gene locus, a PPP1R12C (also known as AAVS1) gene, an albumin gene locus, a SHS231 locus, a CLYBL gene locus, and a ROSA26 gene locus.

In some of any embodiments, the modified SC-beta cell comprises a modification that increases expression of one or more complement inhibitors selected from the group consisting of CD46, CD59, CD55 and CD35 relative to the control or wild-type beta cell. In some of any embodiments the modification to increase expression of the one or more complement inhibitors in the modified SC-beta cell comprises at least one exogenous polynucleotide encoding one or more complement inhibitors selected from the group consisting of an exogenous polynucleotide encoding CD46, an exogenous polynucleotide encoding CD59, an exogenous polynucleotide encoding CD55, and an exogenous polynucleotide encoding CD35. In some of any embodiments, the one or more complement inhibitors is CD46 and CD59. In some of any embodiments, the one or more complement inhibitor is CD46, CD59 and CD55.

In some of any embodiments, the modified SC-beta cell expresses CD47 at a first level that is greater than at or about 5-fold over a second level expressed by the control or wild-type beta cell. In some embodiments, the control or wild-type beta cell is differentiated from an unmodified PSC not comprising modifications that reduce expression of one or more MHC class I molecules and/or one or more MHC class II molecules and that increase expression of the one or more tolerogenic factors. In some of any embodiments, the modified SC-beta cell expresses CD47 at a first level that is greater than at or about 5-fold over a second level expressed by the control or wild-type beta cell. In some of any embodiments, wherein CD47 is expressed at a first level that is greater than at or about 10-fold, greater than at or about 20-fold, greater than at or about 30-fold, greater than at or about 40-fold, greater than at or about 50-fold, greater than at or about 60-fold, or greater than at or about 70-fold over a second level expressed by the control or wild-type beta cell.

In some of any embodiments, CD47 is expressed by the modified SC-beta cell at greater than at or about 20,000 molecules per cell. In some of any embodiments, CD47 is expressed by the modified SC-beta cell at greater than at or about 30,000 molecules per cell, greater than at or about 50,000 molecules per cell, greater than at or about 100,000 molecules per cell, greater than at or about 200,000 molecules per cell, greater than at or about 300,000 molecules per cell, greater than at or about 400,000 molecules per cell, greater than at or about 500,000 molecules per cell, or greater than at or about 600,000 molecules per cell.

In some embodiments of any of the modified SC-beta cells disclosed herein, the modified SC-beta cell comprises an exogenous polynucleotide encoding a suicide gene or a suicide switch. In some embodiments, the suicide gene or suicide switch is selected from the group consisting of cytosine deaminase (CyD), herpesvirus thymidine kinase (HSV-Tk), an inducible caspase 9 (iCaspase9), and rapamycin-activated caspase 9 (rapaCasp9). In some embodiments, the suicide gene or suicide switch and genes associated with the suicide gene or the safety switch are expressed from a bicistronic cassette integrated into the genome of the modified SC-beta cell. In some embodiments, the suicide gene or suicide switch and the one or more tolerogenic factors are expressed from a bicistronic cassette integrated into the genome of the modified SC-beta cell. In some embodiments, the bicistronic cassette is integrated by non-targeted insertion into the genome of the modified SC-beta cell, optionally by introduction of the exogenous polynucleotide into the cell using a lentiviral vector. In some embodiments, the bicistronic cassette is integrated by targeted insertion into a target genomic locus of the cell, optionally wherein the targeted insertion is by nuclease-mediated gene editing with homology-directed repair. In some embodiments, the one or more tolerogenic factors is CD47.

In some of any embodiments, the modified SC-beta cell expresses at least one beta cell marker. In some embodiments, the at least one beta cell marker is selected from the group consisting of INS, CHGA, NKX2-2, PDX1, NKX6-1, MAFB, GCK and GLUT1. In some of any embodiments, the modified SC-beta cell exhibits one or more functions of a wild-type or control beta cell. In some embodiments, the one or more functions is selected from the group consisting of in vitro glucose-stimulated insulin secretion (GSIS), glucose metabolism, maintaining fasting blood glucose levels, secreting insulin in response to glucose injections in vivo, and clearing glucose after a glucose injection in vivo. In some of any embodiments, the GSIS is measured in a perfusion GSIS assay. In some of any embodiments, the GSIS is dynamic GSIS comprising first and second phase dynamic insulin secretion. In some of any embodiments, the GSIS is static GSIS. In some embodiments, the static stimulation index is greater than at or about 1, greater than at or about 1.5, greater than at or about 2, greater than at or about 5, greater than at or about 10, greater than at or about 15, or greater than at or about 20.

In some of any embodiments, the level of insulin secretion by the modified SC-beta cell is at least 20% of that observed for primary beta islets, such as cadaveric islets. In some of any embodiments, the level of insulin secretion by the modified SC-beta cells is at least 25%, at least 30%, at least 40%, at least 50%, at least 60%, at least 70% of that observed for primary beta islets, such as cadaveric islets. In some of any embodiments, the total insulin content of the modified SC-beta is greater than at or about 500 μIU Insulin per 5000 cells, greater than at or about 1000 μIU Insulin per 5000 cells, greater than at or about 2000 μIU Insulin per 5000 cells, greater than at or about 3000 μIU Insulin per 5000 cells or greater than at or about 4000 μIU Insulin per 5000 cells. In some of any embodiments, the proinsulin to insulin ratio of the modified SC-beta is between at or about 0.02 and at or about 0.1, optionally at or about 0.03, 0.04, 0.05, 0.06, 0.07, 0.08, 0.09, and any value between any of the foregoing.

In some of any embodiments, the modified SC-beta cell exhibits functionality for 1 or more days following transplantation into a subject. In some of any embodiments, the modified SC-beta cell exhibits functionality for more than 1 week following transplantation into a subject. In some of any embodiments, the functionality is selected from the group consisting of maintaining fasting blood glucose levels, secreting insulin in response to glucose injections in vivo, and clearing glucose after a glucose injection in vivo.

Provided herein is a composition comprising any of the provided SC-beta cells. Also provided herein is a composition comprising a population of any of the provided modified SC-beta cells.

In some of any embodiments of a provided composition comprising a population of modified SC-beta cells, among the cells in the population, the level of the reduced expression of MHC HLA class I and/or MHC HLA class II and/or the level of the increased expression of the tolerogenic factor is retained or is similar compared to the modified PSC in at least at or about 50%, at least at or about 60%, at least at or about 70%, at least at or about 80%, or at least at or about 90% of the cells in the population. In some of any embodiments, at least at or about 50%, at least at or about 60%, at least at or about 70%, at least at or about 80%, or at least at or about 90% of the cells in the population are reduced for expression of MHC HLA class I or for B2M. In some of any embodiments, at least at or about 50%, at least at or about 60%, at least at or about 70%, at least at or about 80%, or at least at or about 90% of the cells in the population are reduced for expression of MHC HLA class II or for CIITA.

In some of any embodiments of a provided composition comprising a population of modified SC-beta cells, at least at or about 50%, at least at or about 60%, at least at or about 70%, at least at or about 80%, or at least at or about 90% of the cells in the population express the tolerogenic factor at a first level that is greater than at or about 5-fold, greater than at or about 10-fold, greater than at or about 20-fold, greater than at or about 30-fold, greater than at or about 40-fold, greater than at or about 50-fold, greater than at or about 60-fold, or greater than at or about 70-fold over a second level expressed by the control or wild-type beta cell. In some embodiments, the control or wild-type beta cell is a wild-type primary beta cell. In some of any embodiments, at least at or about 50%, at least at or about 60%, at least at or about 70%, at least at or about 80%, or at least at or about 90% of the cells in the population express the tolerogenic factor at a first level that is greater than at or about 5-fold, greater than at or about 10-fold, greater than at or about 20-fold, greater than at or about 30-fold, greater than at or about 40-fold, greater than at or about 50-fold, greater than at or about 60-fold, or greater than at or about 70-fold over a second level expressed by an unmodified PSC not comprising the modifications or an unmodified SC-beta cell differentiated from the unmodified PSC.

In some of any embodiments of a provided composition comprising a population of modified SC-beta cells, at least at or about 50%, at least at or about 60%, at least at or about 70%, at least at or about 80%, or at least at or about 90% of the cells in the population expresses the tolerogenic factor at greater than at or about 20,000 molecules per cell, at greater than at or about 30,000 molecules per cell, greater than at or about 50,000 molecules per cell, greater than at or about 100,000 molecules per cell, greater than at or about 200,000 molecules per cell, greater than at or about 300,000 molecules per cell, greater than at or about 400,000 molecules per cell, greater than at or about 500,000 molecules per cell, or greater than at or about 600,000 molecules per cell.

In some of any embodiments of a provided composition comprising a population of modified SC-beta cells, at least at or about 50%, at least at or about 60%, at least at or about 70%, at least at or about 80%, or at least at or about 90% of the cells in the population are reduced for expression of CD142.

In some of any embodiments, the provided composition comprises a pharmaceutically acceptable excipient. In some of any embodiments, the provided composition comprises a cryoprotectant.

In some embodiments of any of the compositions disclosed herein, modified SC-beta cells of the population of modified SC-beta cells comprise an exogenous polynucleotide encoding a suicide gene or a suicide switch. In some embodiments, the suicide gene or suicide switch is selected from the group consisting of cytosine deaminase (CyD), herpesvirus thymidine kinase (HSV-Tk), an inducible caspase 9 (iCaspase9), and rapamycin-activated caspase 9 (rapaCasp9). In some embodiments, the suicide gene and genes associated with the suicide gene or the safety switch are expressed from a bicistronic cassette integrated into the genome of modified SC-beta cells of the population of modified SC-beta cells. In some embodiments, the suicide gene or suicide switch and the exogenous CD47 are expressed from a bicistronic cassette integrated into the genome of the modified SC-beta cell. In some embodiments, the bicistronic cassette is integrated by non-targeted insertion into the genome, optionally by introduction of the exogenous polynucleotide into modified SC-beta cells of the population of modified SC-beta cells using a lentiviral vector. In some embodiments, the bicistronic cassette is integrated by targeted insertion into a target genomic locus of modified SC-beta cells of the population of modified SC-beta cells, optionally wherein the targeted insertion is by nuclease-mediated gene editing with homology-directed repair.

Provided herein is a method of treating diabetes in a subject, the method comprising administering any of the modified SC-beta cells or any of the provided compositions to a subject in need of treatment thereof.

In some of any embodiments, the diabetes is type I diabetes. In some of any embodiments, the diabetes is type II diabetes. In some of any embodiments, the modified SC-beta cells improve glucose tolerance in the subject.

Provided herein is a method for improving glucose tolerance in a subject, the method comprising administering any of the provided modified SC-beta cells or any of the provided compositions to a subject in need of treatment thereof. In some of any embodiments, the subject is a diabetic patient. In some of any embodiments, the diabetic patient has type I diabetes or type II diabetes.

In some of any embodiments, glucose tolerance is improved relative to the subject's glucose tolerance prior to administration of the modified SC-beta cells. In some of any embodiments, administration of the modified SC-beta cells reduces exogenous insulin usage in the subject. In some of any embodiments, glucose tolerance is improved as measured by HbA1c levels. In some of any embodiments, the subject is fasting. In some of any embodiments, administration of the modified SC-beta cells improves insulin secretion in the subject. In some of any embodiments, insulin secretion is improved relative to the subject's insulin secretion prior to administration of the modified SC-beta cells.

In some embodiments, the method further comprises administering one or more immunosuppressive agents to the subject. In some embodiments, the subject has been administered one or more immunosuppressive agents. In some embodiments, the one or more immunosuppressive agents are a small molecule or an antibody. In some embodiments, the one or more immunosuppressive agents are selected from the group consisting of cyclosporine, azathioprine, mycophenolic acid, mycophenolate mofetil, a corticosteroids, prednisone, methotrexate, gold salts, sulfasalazine, antimalarials, brequinar, leflunomide, mizoribine, 15-deoxyspergualine, 6-mercaptopurine, cyclophosphamide, rapamycin, tacrolimus (FK-506), OKT3, anti-thymocyte globulin, thymopentin (thymosin-α), and an immunosuppressive antibody. In some embodiments, the one or more immunosuppressive agents comprise cyclosporine. In some embodiments, the one or more immunosuppressive agents comprise mycophenolate mofetil. In some embodiments, the one or more immunosuppressive agents comprise a corticosteroid. In some embodiments, the one or more immunosuppressive agents comprise cyclophosphamide. In some embodiments, the one or more immunosuppressive agents comprise rapamycin. In some embodiments, the one or more immunosuppressive agents comprise tacrolimus (FK-506). In some embodiments, the one or more immunosuppressive agents comprise anti-thymocyte globulin. In some embodiments, the one or more immunosuppressive agents are one or more immunomodulatory agents. In some embodiments, the one or more immunomodulatory agents are a small molecule or an antibody. In some embodiments, the antibody binds to one or more of receptors or ligands selected from the group consisting of p75 of the IL-2 receptor, MHC, CD2, CD3, CD4, CD7, CD28, B7, CD40, CD45, IFN-gamma, TNF-alpha, IL-4, IL-5, IL-6R, IL-6, IGF, IGFR1, IL-7, IL-8, IL-10, CD11a, CD58, and antibodies binding to any of their ligands.

In some embodiments, the one or more immunosuppressive agents are or have been administered to the subject prior to administration of the modified SC-beta cells. In some embodiments, the one or more immunosuppressive agents are or have been administered to the subject at least 1, 2, 3, 4, 5, 6, 7, 8, 9, 10, 11, 12, 13, or 14 days prior to administration of the modified SC-beta cells. In some embodiments, the one or more immunosuppressive agents are or have been administered to the subject at least 1 week, 2 weeks, 3 weeks, 4 weeks, 5 weeks, 6 weeks, 7 weeks, 8 weeks, 9 weeks, 10 weeks or more prior to administration of the SC-beta cells. In some embodiments, the one or more immunosuppressive agents are or have been administered to the subject at least 1, 2, 3, 4, 5, 6, 7, 8, 9, 10, 11, 12, 13, or 14 days after administration of the modified SC-beta cells. In some embodiments, the one or more immunosuppressive agents are or have been administered to the subject at least 1 week, 2 weeks, 3 weeks, 4 weeks, 5 weeks, 6 weeks, 7 weeks, 8 weeks, 9 weeks, 10 weeks, or more, after administration of the modified SC-beta cells. In some embodiments, the one or more immunosuppressive agents are or have been administered to the subject on the same day as the first administration of the modified SC-beta cells.

In some embodiments, the one or more immunosuppressive agents are or have been administered to the subject after administration of the modified SC-beta cells. In some embodiments, the one or more immunosuppressive agents are or have been administered to the subject after administration of a first and/or second administration of the modified SC-beta cells. In some embodiments, the one or more immunosuppressive agents are or have been administered to the subject prior to administration of a first and/or second administration of the modified SC-beta cells. In some embodiments, the one or more immunosuppressive agents are or have been administered to the subject at least 1, 2, 3, 4, 5, 6, 7, 8, 9, 10, 11, 12, 13, or 14 days prior to administration of a first and/or second administration of the modified SC-beta cells. In some embodiments, the one or more immunosuppressive agents are or have been administered to the subject at least 1 week, 2 weeks, 3 weeks, 4 weeks, 5 weeks, 6 weeks, 7 weeks, 8 weeks, 9 weeks, 10 weeks or more prior to administration of a first and/or second administration of the modified SC-beta cells. In some embodiments, the one or more immunosuppressive agents are or have been administered to the subject at least 1, 2, 3, 4, 5, 6, 7, 8, 9, 10, 11, 12, 13, or 14 days after administration of a first and/or second administration of the modified SC-beta cells. In some embodiments, the one or more immunosuppressive agents are or have been administered to the subject at least 1 week, 2 weeks, 3 weeks, 4 weeks, 5 weeks, 6 weeks, 7 weeks, 8 weeks, 9 weeks, 10 weeks, or more, after administration of a first and/or second administration of the modified SC-beta cells. In some embodiments, the one or more immunosuppressive agents are administered at a lower dosage compared to the dosage of one or more immunosuppressive agents administered to reduce immune rejection of immunogenic cells that do not comprise the modifications of the modified SC-beta cells.

In some embodiments, the modified SC-beta cell is capable of controlled killing of the modified SC-beta cell. In some embodiments, the modified SC-beta cell comprises a suicide gene or a suicide switch. In some embodiments, the suicide gene or the suicide switch induces controlled cell death in the presence of a drug or prodrug, or upon activation by a selective exogenous compound. In some embodiments, the suicide gene or the suicide switch is an inducible protein capable of inducing apoptosis of the modified SC-beta cell. In some embodiments, the inducible protein capable of inducing apoptosis of the modified SC-beta cell is a caspase protein. In some embodiments, the caspase protein is caspase 9. In some embodiments, the suicide gene or suicide switch is selected from the group consisting of cytosine deaminase (CyD), herpesvirus thymidine kinase (HSV-Tk), an inducible caspase 9 (iCaspase9), and rapamycin-activated caspase 9 (rapaCasp9). In some embodiments, the suicide gene or the suicide switch is activated to induce controlled cell death after the administration of the one or more immunosuppressive agents to the subject. In some embodiments, the suicide gene or the suicide switch is activated to induce controlled cell death prior to the administration of the one or more immunosuppressive agents to the subject. In some embodiments, the suicide gene or the suicide switch is activated to induce controlled cell death after the administration of the modified SC-beta cell to the subject. In some embodiments, the suicide gene or the suicide switch is activated to induce controlled cell death in the event of cytotoxicity or other negative consequences to the subject.

In some embodiments, the method comprises administering an agent that allows for depletion of a modified SC-beta cell of the population of modified SC-beta cells. In some embodiments, the agent that allows for depletion of the modified SC-beta cell is an antibody that recognizes a protein expressed on the surface of the modified SC-beta cell. In some embodiments, the antibody is selected from the group consisting of an antibody that recognizes CCR4, CD16, CD19, CD20, CD30, EGFR, GD2, HER1, HER2, MUC1, PSMA, and RQR8. In some embodiments, the antibody is selected from the group consisting of mogamulizumab, AFM13, MOR208, obinutuzumab, ublituximab, ocaratuzumab, rituximab, rituximab-Rllb, tomuzotuximab, RO5083945 (GA201), cetuximab, Hul4.18K322A, Hul4.18-IL2, Hu3F8, dinituximab, c.60C3-Rllc, and biosimilars thereof. In some embodiments, the method comprises administering an agent that recognizes the one or more tolerogenic factors on the surface of the modified SC-beta cell. In some embodiments, the modified SC-beta cell is engineered to express the one or more tolerogenic factors. In some embodiments, the one or more tolerogenic factors is CD47.

In some embodiments, the method comprises administering one or more additional therapeutic agents to the subject. In some embodiments, the subject has been administered one or more additional therapeutic agents.

In some embodiments, the method comprises monitoring the therapeutic efficacy of the method. In some embodiments, the method comprises monitoring the prophylactic efficacy of the method. In some embodiments, the method is repeated until a desired suppression of one or more disease symptoms occurs.

### BRIEF DESCRIPTION OF THE DRAWINGS

FIG. 1A shows glucose levels measured over time in humanized NSG diabetic mice transplanted with wild-type (WT), B2M^{indel/indel}, CIITA^{indel/indel}, or B2M^{indel/indel} CIITA^{indel/indel}, CD47tg modified SC-beta cells.

FIG. 1B shows serum levels of human c-peptide in humanized NSG diabetic mice transplanted with wild-type (WT), B2M^{indel/indel}, CIITA^{indel/indel}, or B2M^{indel/indel}, CIITA^{indel/indel}, CD47tg modified SC-beta cells and subjected to a glucose challenge on da 29 after transplant, one hour prior to sacrifice.

FIG. 2A shows IFN-γ spot frequencies enumerated using an Elispot plate reader as an assay for TH1 T cell response in mice administered wild-type, B2M^{indel/indel}, CIITA^{indel/indel}, or B2M^{indel/indel} CIITA^{indel/indel}, CD47tg human SC-beta cells.

FIG. 2B shows the mean fluorescence intensity (MFI) of cells labelled with FITC-conjugated goat anti-IgM and analyzed by flow cytometry for cell suspensions of sera from recipient wild-type, B2M^{indel/indel}, CIITA^{indel/indel}, and B2M^{indel/indel}, CIITA^{indel/indel}, CD47tg human SC-beta mice incubated with wild-type, B2M^{indel/indel}, CIITA^{indel/indel}, and B2M^{indel/indel}, CIITA^{indel/indel}, CD47tg human cells.

FIG. 2C shows a comparison of NK cell killing using IL-2 stimulated human NK cells as effector and wild-type, B2M^{indel/indel}, CIITA^{indel/indel}, or B2M^{indel/indel}, CIITA^{indel/indel}, CD47tg human SC-beta cells as target cells.

### DETAILED DESCRIPTION

Provided herein are beta cells differentiated from pluripotent stem cells (PSCs) in which the resulting beta cells contain one or more modifications that make the resulting differentiated beta cells hypoimmune to reduce or evade immune rejection. The modified beta cells that have been differentiated in vitro from PSCs and that contain the one more more modifications are called modified stem cell-derived beta cell (also called “modified SC-beta cell” or “modified SC-β cell”). In some cases, at least one or all of the modifications to the differentiated beta-cell are introduced to the beta-cells after the diffierentiation from the PSCs. In some cases, the PSCs are first modified with the one or more modifications, or in some embodiments each of the hypoimmune modifications, and then are differentiated to generate the modified SC-beta cells. For purposes herein, the terms modified SC-beta cells and hypoimmune PSC derived beta cells (HIP beta cells) can be used interchangeably.

Provided herein is a method of generating a hypoimmune beta cell differentiated from a modified pluripotent stem cell (PSC) in vitro that has been modified to evade immune rejection. In some embodiments, these modified pluripotent stem cells can also encompass mesenchymal stem cells (MSCs) and/or embryonic stem cells (ESCs). In some embodiments, the modifications that result in hypoimmune cells are modifications that inactivate or disrupt one or more alleles (e.g. one or both alleles) of one or more major histocompatibility complex (MHC) human leukocyte antigen MHC class I antigens and/or MHC class II antigens, or that inactivate or disrupt one or more alleles (e.g. one or both alleles) of one or more molecules that regulate expression, such as surface expression, of the one or more MHC class I and/or class II molecules in the modified cell. Non-limiting examples of modifications that result in evading immune rejection reduce expression of major histocompatibility complex (MHC) class I antigens and MHC class II antigens (also called human leukocyte antigen (HLA) class I antigens and HLA class II antigens, respectively), and increased expression of one or more tolerogenic factors, such as CD47. The in vitro differentiated beta cell that has been differentiated from a modified PSC is an example of a modified stem cell-derived beta cell.

In some embodiments, the provided cells are modified SC-beta cells that are derived from modified PSCs that contain modifications that (a) reduce expression of one or more major histocompatibility complex (MHC) class I molecules and/or one or more of MHC class II molecules; and (b) increase expression of one or more tolerogenic factors in the modified PSC, relative to a control or a wild-type PSC. In some embodiments, the modified SC-beta cells are derived from the modified PSC by culture under conditions sufficient for differentiation of the modified PSC into a modified SC-beta cell. In some embodiments, the modifications of the modified SC-beta cells make the cells hypoimmune, which in some aspects allow the cells to evade immune rejections compared to control or wild-type beta cells, such as primary human beta cells. The provided embodiments relate to a demonstration of differentiating functional modified SC-beta cells from hypoimmune PSCs in which the modified SC-beta cells retain the hypoimmune modifications of the PSC and exhibit beta cell function such as glucose-stimulated insulin secretion. These results support that the hypoimmune modifications do not impact the differentiation of PSCs into functional beta cells.

The provided embodiments provide for a viable source of a transplantable beta cell, and thus provide for an allogeneic cell therapy for improving glucose tolerance in diabetic subjects. In aspects of the modified SC-beta cells provided herein, rejection of the cells by the recipient subject's immune system is diminished and the cells are able to engraft and function in the host after their administration, regardless of the subject's genetic make-up, or any existing response within the subject to one or more previous allogeneic transplants. In some embodiments, the modified SC-beta cells are able to persist without immunosuppression. In a specific embodiments, the modified SC-beta cells are able to persist without immunosuppression course used in allogenic islet transplantation. In certain embodiments, the cells are genomically stable with respect to the modifications present in the iPSC from which the modified SC-beta cell is differentiated.

The modified cells provided herein, including modified PSCs and modified SC-beta cells (e.g., modified SC-beta cells obtained by in vitro differentiation from the modified PSCs), utilize expression of tolerogenic factors and are also modulated (e.g., reduced or eliminated) for expression (e.g., surface expression) of one or more MHC class I molecules and/or one or more MHC class II molecules. In some embodiments, the modification that reduces expression of one or more MHC class I molecules is a modification that reduces expression of b-2 microglobulin (B2M). In some embodiments, the modification that reduces expression of one or more MHC class II molecules is a modification that reduces expression of CIITA. In some embodiments, the modified SC-beta cells comprising the modifications described herein (including reduced or eliminated expression of MHC class I molecules or MHC class II molecules and increased expression of CD47 or other tolerogenic factor) survive, engraft, persist, and function following transplant. In some embodiments, the modified SC-beta cells exhibit enhanced survival and/or enhanced engraftment and/or function for a longer term in comparison to control or wild-type beta cells, such as unmodified SC-beta cells that do not comprise the modifications, such as SC-beta cells differentiated from unmodified PSCs that do not contain the modifications rendering the cells hypoimmune. In some embodiments, the modified SC-beta cells are administered via intravenous infusion, intramuscular injection, or kidney capsule transplant.

In certain embodiments, provided herein are methods of generating a modified SC-beta cell that is hypoimmune, in which the methods include (1) providing a modified PSC with one or more modifications (e.g. genetic modifications) that reduce or eliminate expression of one or more MHC class I molecules (e.g. via reduced or eliminated B2M) and/or one or more MHC class II human leukocyte antigens (e.g. via reduced or eliminated CIITA) in the modified PSC and increase expression of a tolerogenic factor (e.g. CD47) in the modified PSC, and (2) differentiating the modified PSC under conditions for differentiation into a beta islet cell. Thus, also provided herein are modified SC-beta cells that are obtained by the method. Also provided herein are modified SC-beta cells obtained by differentiation in vitro from a modified PSC that has one or more modifications (e.g., genetic modifications) to reduce or eliminate expression of one or more MHC class I molecules and/or one or more MHC class II molecules in the modified PSC and increase expression of a tolerogenic factor (e.g., CD47) in the cell. In provided embodiments, the resulting or obtained modified SC-beta cell also has reduced or eliminated expression of the one or more MHC class I molecules (e.g. via reduced or eliminated B2M) and/or the one or more MHC class II molecules (e.g. via reduced or eliminated CIITA) and increased expression of a tolerogenic factor (e.g. CD47), such as compared to a unmodified PSC, including the starting pluripotent stem cell line, or compared to a control or wild-type beta cell such as an SC-beta cell differentiated from an unmodified PSC. In some embodiments, the modified SC-beta cells do not express MHC class I molecules and/or MHC class II molecules, and express CD47 at increased levels relative to the starting cell line (e.g., greater than 5-fold over background, greater than 5-fold over a primary beta cell, and/or greater than 5-fold compared to an unmodified PSC or an unmodified SC-beta cell obtained by in vitro differentiation from the unmodified PSC). In some embodiments, the modified SC-beta cell expresses CD47 at a level over the expression by the endogenous iPSC and/or beta cell differentiated therefrom. In some embodiments, the modified PSC or modified SC-beta cell expresses greater than 20,000 molecules of the tolerogenic factor (e.g., CD47) on its surface. Also provided herein are compositions containing the modified SC-beta cells and methods and uses thereof for treating diabetic subjects and/or for improving glucose tolerance in subjects in need thereof.

In some embodiments, the modified PSCs (e.g., modified iPSC) from which the modified SC-beta cells are differentiated from further comprise reduced or eliminated expression of CD142 (also known as Coagulation Factor III, Tissue Factor (TF), Thromboplastin, platelet tissue factor, or factor III), which is a membrane receptor in the blood coagulation pathway that contributes to initiating IBMIR. In some embodiments, the modified PSCs comprise reduced or eliminated for expression of CD142 and increased expression of one or more tolerogenic factors (e.g., CD47), and reduced expression of one or more MHC class I molecules and/or MHC class II molecules, such as described above. Also provided are modified SC-beta cells that are obtained by in vitro differentiation from such modified PSCs. In some embodiments, the modified SC-beta cell obtained by in vitro differentiation from a modified PSC is reduced or eliminated for expression of CD142, reduced or eliminated for expression of MHC class I molecules and/or MHC class II molecules and increased for the expression of a tolerogenic factor (e.g. CD47), such as compared to an unmodified PSC or compared to a control or wild-type beta cell such as an SC-beta cell differentiated from an unmodified PSC.

In some embodiments, the modified PSCs (e.g., modified iPSC) from which the modified SC-beta cells are differentiated from as described herein further comprise increased expression and/or overexpression of one or more complement inhibitors. In some embodiments, the one or more complement inhibitors are selected from CD46, CD59, CD55 and CD35. In some embodiments, the modified PSCs comprise increased expression of one or more complement inhibitors and increased expression of one or more tolerogenic factors (e.g., CD47), and reduced expression of one or more MHC class I molecules and/or MHC class II molecules, such as described above. In some embodiments, the modified cells comprise increased expression of two or more complement inhibitors in combination, such as increased expression of CD46 and CD59 or increased expression of CD46, CD59, and CD55. Also provided are modified SC-beta cells that are obtained by in vitro differentiation from such modified PSCs. Also provided are modified SC-beta cells that are obtained by in vitro differentiation from such modified PSCs. In some embodiments, the modified SC-beta cell obtained by in vitro differentiation from a modified PSC is reduced or eliminated for expression of one or more MHC class I molecules and/or one or more MHC class II molecules, increased for the expression of a tolerogenic factor (e.g. CD47), and increased for expression of one or more complement inhibitors from CD46, CD59, CD55 and CD35 (e.g. CD46 and CD59 or CD46, CD59 and CD55), such as compared to an unmodified PSC or compared to a control or wild-type beta cell such as a SC-beta cell differentiated from an unmodified PSC.

In some embodiments, the modified SC-beta cells provided herein are protected from complement-mediated cytotoxicity. In some embodiments, the modified SC-beta cells (e.g., overexpressing one or more complement inhibitors, such as CD46 and CD59) are protected from complement-dependent cytotoxicity (CDC) that occurs as a result of an IBMIR. In some embodiments, the modified SC-beta cells are protected from CDC that occurs independently of an IBMIR.

In any of the provided embodiments, the altered expression is relative to a similar cell that does not contain the modifications, such as a wild-type cell, the starting cell line to which the modifications are made, or an unmodified cell of the same cell type or a cell that otherwise is the same but that lacks the modifications. It is understood that a cell that lacks the modifications is any cell as described herein that lacks modifications herein to alter expression of the one or more tolerogenic factors (e.g., CD47), one or more MHC class I molecule and/or one or more MHC class II molecule, CD142 and/or one or more complement inhibitor. Exemplary methods to introduce modifications to a cell to alter expression are described herein. For instance, any of a variety of methods for overexpressing or increasing expression of a gene or protein in a pluripotent stem cell may be used, such as by introduction or delivery of an exogenous polynucleotide encoding a protein (i.e., a transgene or “tg”) or introduction of delivery of a fusion protein of a DNA-targeting domain and a transcriptional activator targeting a gene. Also, any of a variety of methods for reducing or eliminating expression of a gene or protein in a PSC may be used, including non-gene editing methods such as by introduction or delivery of an inhibitory nucleic acids (e.g., RNAi) or gene editing methods involving introduction or delivery of a targeted nuclease system (e.g., CRISPR/Cas). In some embodiments, the method for reducing or eliminating expression is via a nuclease-based gene editing technique. The PSC may then be used to differentiate a modified SC-beta cell that then also is found to contain the similar modifications. Hence, it is understood by this disclosure that description related to editing or modification of a cell relates to editing or modification of the pluripotent stem cell, and that the modified SC-beta cell is derived from such modified pluripotent stem cell by in vitro differentiation therefrom.

In some embodiments, genome editing technologies utilizing rare-cutting endonucleases (e.g., the CRISPR/Cas, TALEN, zinc finger nuclease, meganuclease, and homing endonuclease systems) are used to reduce or eliminate expression of immune genes (e.g., by deleting genomic DNA of critical immune genes) as described herein, such as genes involved in regulating expression of MHC class I molecules or MHC class II molecules, in PSCs used to derived the modified SC-beta cells. In certain embodiments, genome editing technologies or other gene modulation technologies are used to insert tolerance-inducing (tolerogenic) factors (e.g., CD47) into a target genomic locus of PSCs used to derive the modified SC-beta cells, thus producing modified cells that can evade immune recognition upon engrafting into a recipient subject. Therefore, the modified PSCs, and modified SC-beta cells (e.g., modified SC-beta cells obtained by in vitro differentiation of the modified PSCs), provided herein exhibit modulated expression (e.g., reduced or eliminated expression) of one or more genes and factors that affect expression of MHC class I molecules and/or MHC class II molecules, modulated expression (e.g., reduced or and modulated expression (e.g., overexpression) of tolerogenic factors, such as CD47, and provide for reduced recognition by the recipient subject's immune system. In some embodiments, the modified cells provided herein also exhibit modulated expression (e.g., reduced expression) of CD142, which, in some aspects, can also be reduced by genome editing technologies (e.g., the CRISPR/Cas, TALEN, zinc finger nuclease, meganuclease, and homing endonuclease systems) to reduce or eliminate expression of CD142 (e.g., by deleting genomic DNA of critical immune genes) in modified PSCs used to derived the modified SC-beta cells. In some embodiments, the modified SC-beta cells provided herein also exhibit modulated expression (e.g., reduced expression) of CD142, which, in some aspects, can also be reduced by genome editing technologies (e.g., the CRISPR/Cas, TALEN, zinc finger nuclease, meganuclease, and homing endonuclease systems) to reduce or eliminate expression of CD142 (e.g., by deleting genomic DNA of critical immune genes) in modified SC-beta cells.

In some embodiments, the modified cells provided herein exhibit modulated expression (e.g., increased expression) of one or more complement inhibitors selected from CD46, CD59, CD55 and CD35, which, in some aspect, also can be increased by genome editing technologies to insert or integrate an exogenous polynucleotide encoding the one or more complement inhibitors into a genomic locus in modified PSCs used to derive the modified SC-beta cells. In some embodiments, modulated expression (e.g., increased expression) of one or more complement inhibitors selected from CD46, CD59, CD55 and CD35 is increased by genome editing technologies to insert or integrate an exogenous polynucleotide encoding the one or more complement inhibitors into a genomic locus in modified SC-beta cells.

In some embodiments, the modified SC-beta cells exhibit features that allow them to evade immune recognition. In some embodiments, the provided modified SC-beta cells are hypoimmunogenic. In some aspects, modified SC-beta cells provided herein are not subject to an innate immune cell rejection. In some aspects, modified SC-beta cells provided herein exhibit reduced innate immune cell rejection and/or adaptive immune cell rejection (e.g., hypo-immunogenic cells). For example, in some embodiments, the modified SC-beta cells exhibit reduced susceptibility to NK cell-mediated lysis and/or macrophage engulfment. In some embodiments, the modified SC-beta cells are useful as a source of universally compatible cells or tissues (e.g., universal donor cells or tissues) that are transplanted into a recipient subject with little to no immunosuppressant agent needed. Such hypo-immunogenic cells retain cell-specific characteristics and features upon transplantation.

Also provided herein are methods for treating a disorder comprising administering the modified cells that evade immune rejection in an MHC-mismatched allogenic recipient. In some embodiments, the modified cells produced from any one of the methods described herein evade immune rejection when repeatedly administered (e.g., transplanted or grafted) to MHC-mismatched allogenic recipient. In particular embodiments, the modified SC-beta cells are used in methods for treating diabetic subjects (e.g., Type I or Type II diabetes), such as to improve glucose tolerance in the subject.

The practice of the particular embodiments will employ, unless indicated specifically to the contrary, conventional methods of chemistry, biochemistry, organic chemistry, molecular biology, microbiology, recombinant DNA techniques, genetics, immunology, and cell biology that are within the skill of the art, many of which are described below for the purpose of illustration. Such techniques are explained fully in the literature. See e.g., Sambrook, et al., Molecular Cloning: A Laboratory Manual (3rd Edition, 2001); Sambrook, et al., Molecular Cloning: A Laboratory Manual (2nd Edition, 1989); Maniatis et al., Molecular Cloning: A Laboratory Manual (1982); Ausubel et al., Current Protocols in Molecular Biology (John Wiley and Sons, updated July 2008); Short Protocols in Molecular Biology: A Compendium of Methods from Current Protocols in Molecular Biology, Greene Pub. Associates and Wiley-Interscience; Glover, DNA Cloning: A Practical Approach, vol. I & II (IRL Press, Oxford, 1985); Anand, Techniques for the Analysis of Complex Genomes, (Academic Press, New York, 1992); Transcription and Translation (B. Hames & S. Higgins, Eds., 1984); Perbal, A Practical Guide to Molecular Cloning (1984); Harlow and Lane, Antibodies, (Cold Spring Harbor Laboratory Press, Cold Spring Harbor, N.Y., 1998) Current Protocols in Immunology Q. E. Coligan, A. M. Kruisbeek, D. H. Margulies, E. M. Shevach and W. Strober, eds., 1991); Annual Review of Immunology; as well as monographs in journals such as Advances in Immunology.

All publications, including patent documents, scientific articles, and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described. Those skilled in the art will recognize that several embodiments are possible within the scope and spirit of the present disclosure. The following description illustrates the disclosure and, of course, should not be construed in any way as limiting the scope of the inventions described herein.

### I. Pluripotent Stem Cells

In some aspects, provided herein is a modified stem-cell derived beta cell (SC-beta cell). In some embodiments, the modified SC-beta cell is produced by differentiating a stem or progenitor cell (e.g., a totipotent, pluripotent, or multipotent stem cell) into an SC-beta cell, and then generating a modified SC-beta cell from the SC-beta cell. In some embodiments, the modified SC-beta cell is produced from the SC-beta cell by introducing one or more of the modifications disclosed herein. In some embodiments, the SC-beta cell is differentiated from a stem cell (e.g., a PSC such as an iPSC) comprising one or more of the modifications, and one or more additional modifications are introduced into the SC-beta cell to generate the modified SC-beta cell. In some embodiments, the modified SC-beta cell is differentiated from a stem cell (e.g., a PSC such as an iPSC) comprising the modifications.

A. Pluripotent Stem Cells (e.g. iPSCs) Cells and Methods of Producing

The modified stem-cell derived beta cells (SC-beta cells) provided herein can be differentiated from stem or progenitor cells. In some embodiments, the stem or progenitor cells are modified. In some embodiments, the stem or progenitor cell does not comprise the modifications, and the one or more modifications are introduced into the SC-beta cell to generate the modified SC-beta cell. In some embodiments, the cell to be engineered or modified is a stem or progenitor cell that is capable of being differentiated (e.g., the stem cell is totipotent, pluripotent, or multipotent). In some embodiments, a stem cell capable of being differentiated (e.g., the stem cell is totipotent, pluripotent, or multipotent) is differentiated into an SC-beta cell, which is then modified. In some embodiments, the cell is isolated from embryonic or neonatal tissue. In some embodiments, the cell is an embryonic stem cell. In some embodiments, the cell is an induced pluripotent stem cell derived from somatic cells (e.g., skin or blood cells) and reprogrammed into an embryonic-like pluripotent state. In some embodiments, the induced pluripotent stem cell is derived from a fibroblast. In some embodiments, the cells that are modified as provided herein are pluripotent stems cells or are cells differentiated from pluripotent stem cells. The cell may be a vertebrate cell, for example, a mammalian cell, such as a human cell or a mouse cell. The cell may also be a vertebrate stem cell, for example, a mammalian stem cell, such as a human stem cell or a mouse stem cell. Preferably, the cell or stem cell is amenable to modification. Preferably, the cell or stem cell, or a cell derived from such a stem cell, has or is believed to have therapeutic value, such that the cell or stem cell or a cell derived or differentiated from such stem cell may be used to treat a disease, disorder, defect or injury in a subject in need of treatment for same.

In some embodiments, the modified SC-beta cell is differentiated from a pluripotent stem cell, such as an induced pluripotent stem cell (iPSC), optionally wherein the iPSC is modified as disclosed herein. In some embodiments, the iPSC does not comprise the modifications. In some embodiments, the cells that are modified as provided herein are modified pluripotent stem cells (e.g., modified iPSC).

The generation of mammalian (e.g., mouse and human) pluripotent stem cells (generally referred to as iPSCs; miPSCs for murine cells or hiPSCs for human cells) is generally known in the art. As will be appreciated by those in the art, there are a variety of different methods for the generation of iPCSs. The original induction was done from mouse embryonic or adult fibroblasts using the viral introduction of four transcription factors, Oct3/4, Sox2, c-Myc and Klf4; see Takahashi and Yamanaka Cell 126:663-676 (2006), hereby incorporated by reference in its entirety and specifically for the techniques outlined therein. Since then, a number of methods have been developed; see Seki et al, World J. Stem Cells 7 (1): 116-125 (2015) for a review, and Lakshmipathy and Vermuri, editors, Methods in Molecular Biology: Pluripotent Stem Cells, Methods and Protocols, Springer 2013, both of which are hereby expressly incorporated by reference in their entirety, and in particular for the methods for generating hiPSCs (see for example Chapter 3 of the latter reference).

Generally, iPSCs are generated by the transient expression of one or more reprogramming factors” in the host cell, usually introduced using episomal vectors. Under these conditions, small amounts of the cells are induced to become iPSCs (in general, the efficiency of this step is low, as no selection markers are used). Without wishing to be bound by theory, it is believed that once the cells are “reprogrammed”, and become pluripotent, they lose the episomal vector(s) and produce the factors using the endogenous genes.

As is also appreciated by those of skill in the art, the number of reprogramming factors that can be used or are used can vary. Commonly, when fewer reprogramming factors are used, the efficiency of the transformation of the cells to a pluripotent state goes down, as well as the “pluripotency”, e.g., fewer reprogramming factors may result in cells that are not fully pluripotent but may only be able to differentiate into fewer cell types.

In some embodiments, a single reprogramming factor, OCT4, is used. In other embodiments, two reprogramming factors, OCT4 and KLF4, are used. In other embodiments, three reprogramming factors, OCT4, KLF4 and SOX2, are used. In other embodiments, four reprogramming factors, OCT4, KLF4, SOX2 and c-Myc, are used. In other embodiments, 5, 6 or 7 reprogramming factors can be used selected from SOKMNLT; SOX2, OCT4 (POU5F1), KLF4, MYC, NANOG, LIN28, and SV40L T antigen. In general, these reprogramming factor genes are provided on episomal vectors such as are known in the art and commercially available.

In some embodiments, the hosts cells used for transfecting the one or more reprogramming factors are non-pluripotent stem cells. In general, as is known in the art, iPSCs are made from non-pluripotent cells such as, but not limited to, blood cells, fibroblasts, etc., by transiently expressing the reprogramming factors as described herein. In some embodiments, the non-pluripotent cells, such as fibroblasts, are obtained or isolated from one or more individual subjects or donors prior to reprogramming the cells. In some embodiments, iPSCs are made from a pool of isolated non-pluripotent stems cells, e.g., fibroblasts, obtained from one or more (e.g. two or more, three or more, four or more, five or more, ten or more, twenty or more, fifty or more, or one hundred or more) different donor subjects. In some embodiments, the non-pluripotent cells, such as fibroblasts, are isolated or obtained from a plurality of different donor subjects (e.g., two or more, three or more, four or more, five or more, ten or more, twenty or more, fifty or more, or one hundred or more), pooled together in a batch, reprogrammed as iPSCs, and are optionally modified in accord with the provided methods. In some embodiments, the non-pluripotent cells, such as fibroblasts, are isolated or obtained from a plurality of different donor subjects (e.g., two or more, three or more, four or more, five or more, ten or more, twenty or more, fifty or more, or one hundred or more), pooled together in a batch, reprogrammed as iPSCs, and differentiated into SC-beta cells, which are then modified in accord with the provided methods.

In some embodiments, the iPSCs are derived from, such as by transiently transfecting one or more reprogramming factors into cells from a pool of non-pluripotent cells (e.g., fibroblasts) from one or more donor subjects that are different than the recipient subject (e.g., the patient administered the cells). The non-pluripotent cells (e.g., fibroblasts) to be induced to iPSCs can be obtained from 1, 2, 3, 4, 5, 6, 7, 8, 9, 10, 20, 50, 100 or more donor subjects and pooled together. The non-pluripotent cells (e.g., fibroblasts) can be obtained from 1 or more, 2 or more, 3 or more, 4 or more, 5 or more, 6 or more, 7 or more, 8 or more, 9 or more, 10, or more 20 or more, 50 or more, or 100 or more donor subjects and pooled together. In some embodiments, the non-pluripotent cells (e.g., fibroblasts) are harvested from one or a plurality of individuals, and in some instances, the non-pluripotent cells (e.g., fibroblasts) or the pool of non-pluripotent cells (e.g., fibroblasts) are cultured in vitro and transfected with one or more reprogramming factors to induce generation of iPSCs. In some embodiments, the non-pluripotent cells (e.g., fibroblasts) or the pool of non-pluripotent cells (e.g., fibroblasts) are modified in accord with the methods provided herein. In some embodiments, the iPSCs (e.g., modified iPSCs) or a pool of iPSCs (e.g., a pool of modified iPSCs) are then subjected to a differentiation process for differentiation into any cells of an organism and tissue.

Any of the pluripotent stem cells described herein can be differentiated into any cells of an organism and tissue. In an aspect, provided herein are pluripotent stem cells (e.g., modified pluripotent stem cells) that are differentiated into different cell types from iPSCs for subsequent transplantation into recipient subjects. Differentiation can be assayed as is known in the art, generally by evaluating the presence of cell-specific markers. As will be appreciated by those in the art, the differentiated SC-beta cells generated from PSCs such as modified (e.g., hypoimmunogenic) pluripotent cell derivatives can be transplanted using techniques known in the art that depends on both the cell type and the ultimate use of these cells. Exemplary types of differentiated cells and methods for producing the same are described below. In some embodiments, the iPSCs may be differentiated to any type of cell described herein. In some embodiments, the iPSCs are differentiated into beta islet cells. In some embodiments, host cells such as non-pluripotent cells (e.g., fibroblasts) from an individual donor or a pool of individual donors are isolated or obtained, generated into iPSCs in which the iPSCs are then modified to contain modifications (e.g., genetic modifications) described herein and then differentiated into a desired cell type. In some embodiments, host cells such as non-pluripotent cells (e.g., fibroblasts) from an individual donor or a pool of individual donors are isolated or obtained, generated into iPSCs in which the iPSCs are then then differentiated into a desired cell type, which is then modified.

In some embodiments, the cells as provided herein are beta islet cells derived from iPSCs, such as modified iPSCs that contain modifications (e.g., genetic modifications) described herein and that are differentiated into beta islet cells. As will be appreciated by those in the art, the methods for differentiation depend on the desired cell type using known techniques. In some embodiments, the cells differentiated into various beta islet cells may be used for subsequent transplantation or engraftment into subjects (e.g., recipients).

In some embodiments, pancreatic islet cells are derived from the pluripotent cells (e.g., modified pluripotent cells) described herein. Useful methods for differentiating pluripotent stem cells into beta islet cells are described, for example, in U.S. Pat. Nos. 9,683,215; 9,157,062; 8,927,280; U.S. Patent Pub. No. 2021/0207099; Hogrebe et al., “Targeting the cytoskeleton to direct pancreatic differentiation of human pluripotent stem cells,” Nat. Biotechnol., 2020, 38:460-470; and Hogrebe et al., “Generation of insulin-producing pancreatic beta cells from multiple human stem cell lines,” Nat. Protoc., 2021, the contents of which are herein incorporated by reference in their entirety,

In some embodiments, the pluripotent cells (e.g., modified pluripotent cells) described herein are differentiated into beta-like cells or islet organoids for transplantation to address type I diabetes mellitus (T1DM). Cell systems are a promising way to address T1DM, see, e.g., Ellis et al, Nat Rev Gastroenterol Hepatol. 2017 October; 14 (10): 612-628, incorporated herein by reference. Additionally, Pagliuca et al. (Cell, 2014, 159 (2): 428-39) reports on the successful differentiation of beta-cells from hiPSCs, the contents incorporated herein by reference in its entirety and in particular for the methods and reagents outlined there for the large-scale production of functional human beta cells from human pluripotent stem cells). Furthermore, Vegas et al. shows the production of human beta cells from human pluripotent stem cells followed by encapsulation to avoid immune rejection by the host; Vegas et al., Nat Med, 2016, 22 (3): 306-11, incorporated herein by reference in its entirety and in particular for the methods and reagents outlined there for the large-scale production of functional human β cells from human pluripotent stem cells.

In some embodiments, the method of producing a population of modified pancreatic islet cells from a population of pluripotent cells (e.g., modified pluripotent cells) by in vitro differentiation comprises: (a) culturing the population of iPSCs (e.g., modified iPSCs) in a first culture medium comprising one or more factors selected from the group consisting insulin-like growth factor, transforming growth factor, FGF, EGF, HGF, SHH, VEGF, transforming growth factor-b superfamily, BMP2, BMP7, a GSK inhibitor, an ALK inhibitor, a BMP type 1 receptor inhibitor, and retinoic acid to produce a population of immature pancreatic islet cells; and (b) culturing the population of immature pancreatic islet cells in a second culture medium that is different than the first culture medium to produce a population of pancreatic islet cells (e.g., modified pancreatic islet cells). In some embodiments, the method comprise introducing one or more modifications into the pancreatic islet cells. In some embodiments, the GSK inhibitor is CHIR-99021, a derivative thereof, or a variant thereof. In some instances, the GSK inhibitor is at a concentration ranging from about 2 mM to about 10 mM. In some embodiments, the ALK inhibitor is SB-431542, a derivative thereof, or a variant thereof. In some instances, the ALK inhibitor is at a concentration ranging from about 1 pM to about 10 pM. In some embodiments, the first culture medium and/or second culture medium are absent of animal serum.

Differentiation is assayed as is known in the art, generally by evaluating the presence of β cell associated or specific markers, including but not limited to, insulin. Differentiation can also be measured functionally, such as measuring glucose metabolism, see generally Muraro et al., Cell Syst. 2016 Oct. 26; 3 (4): 385-394.e3, hereby incorporated by reference in its entirety, and specifically for the biomarkers outlined there. Once the beta cells are generated, they can be transplanted (either as a cell suspension, cell clusters, or within a permeable or semipermeable device or gel matrix as discussed herein) into the portal vein/liver, the omentum, the gastrointestinal mucosa, the bone marrow, a muscle, or subcutaneous pouches.

Additional descriptions of pancreatic islet cells including for use in the present technology are found in WO2020/018615, the disclosure is herein incorporated by reference in its entirety.

In some embodiments, the population of modified beta islet cells, such as endothelial cells differentiated from iPSCs derived from one or more individual donors (e.g., healthy donors), are maintained in culture, in some cases expanded, prior to administration. In certain embodiments, the population of modified beta islet cells are cryopreserved prior to administration.

Exemplary pancreatic islet cell types include, but are not limited to, pancreatic islet progenitor cell, immature pancreatic islet cell, mature pancreatic islet cell, and the like. In some embodiments, pancreatic cells described herein are administered to a subject to treat diabetes.

In some embodiments, the pancreatic islet cells modified as disclosed herein, such as beta islet cells differentiated from iPSCs derived from one or more individual donors (e.g., healthy donors), secretes insulin. In some embodiments, a pancreatic islet cell exhibits at least two characteristics of an endogenous pancreatic islet cell, for example, but not limited to, secretion of insulin in response to glucose, and expression of beta cell markers.

Exemplary beta cell markers or beta cell progenitor markers include, but are not limited to, c-peptide, Pdxl, glucose transporter 2 (Glut2), HNF6, VEGF, glucokinase (GCK), prohormone convertase (PC 1/3), Cdcpl, NeuroD, Ngn3, Nkx2.2, Nkx6.1, Nkx6.2, Pax4, Pax6, Ptfla, Isll, Sox9, Soxl7, and FoxA2.

In some embodiments, the pancreatic islet cells, such as beta islet cells differentiated from iPSCs derived from one or more individual donors (e.g., healthy donors), produce insulin in response to an increase in glucose. In various embodiments, the pancreatic islet cells secrete insulin in response to an increase in glucose. In some embodiments, the cells have a distinct morphology such as a cobblestone cell morphology and/or a diameter of about 17 pm to about 25 pm.

In some embodiments, the present technology is directed to modified beta islet cells, such as beta islet cells differentiated from iPSCs derived from one or more individual donors (e.g., healthy donors), that overexpress a tolerogenic factor (e.g., CD47), have reduced expression or lack expression of MHC class I and/or MHC class II human leukocyte antigens, and optionally have reduced CD142 expression. In some embodiments, the beta islet cells further express one or more complement inhibitors. In certain embodiments, the modified beta islet cells overexpress a tolerogenic factor (e.g., CD47) and harbor a genomic modification in the B2M gene and optionally have reduced CD142 expression. In some embodiments, the beta islet cells further express one or more complement inhibitors. In some embodiments, the modified beta islet cells overexpress a tolerogenic factor (e.g., CD47) and harbor a genomic modification in the CIITA gene, and optionally have reduced CD142 expression. In some embodiments, the beta islet cells further express one or more complement inhibitors. In some embodiments, beta islet cells overexpress a tolerogenic factor (e.g., CD47) and harbor genomic modifications that disrupt one or more of the following genes: the B2M CIITA, and CD142 genes.

In some embodiments, the provided modified beta islet cells evade immune recognition. In some embodiments, the modified beta islet cells described herein, such as beta islet cells differentiated from iPSCs derived from one or more individual donors (e.g., healthy donors), do not activate an immune response in the patient (e.g., recipient upon administration). Provided are methods of treating a disease by administering a population of modified beta islet cells described herein to a subject (e.g., recipient) or patient in need thereof.

In some embodiments, the number of cells administration is at a lower dosage than would be required for immunogenic cells (e.g., a population of cells of the same or similar cell type or phenotype but that do not contain the modifications, e.g., genetic modifications, of the modified cells, e.g. with endogenous levels of CD142, MHC class I, and/or MHC class II expression and without increased (e.g., exogenous) expression of CD47).

B. Modified Pluripotent Stem Cells (e.g., Modified iPSCs) and Methods of Making

In some embodiments, the PSCs that are differentiated into beta cells, such as methods as described above, are modified pluripotent stem cells or modified PSCs. In some aspects, provided herein are pluripotent stem cells that comprise one or more modification (termed “modified pluripotent stem cells”) in which the one or more modification modulates or regulates the expression of one or more target polynucleotide sequences involved in evading or alleviating an immune response. In some embodiments, the PSCs, such as modified PCSs, are induced pluripotent stem cells (also called “iPSCs,” such as “modified iPSCs”). In some embodiments, the one or more modifications modulate or regulate (e.g., reduce or eliminate) the expression of MHC class I molecules, MHC class II molecules, or MHC class I and MHC class II molecules. In some embodiments, the one or more modifications modulate or regulate (e.g., increase) the expression of a tolerogenic factor, such as CD47. In some embodiments, one or more other modifications that modulate or regulate expression of other immune molecules also can be present in the modified pluripotent stem cells, such as a modification that regulates (e.g., reduces or eliminates) the expression of CD142 or a modification that regulates (e.g., increases) the expression of one or more complement inhibitor.

In some embodiments, the provided modified pluripotent stem cells (e.g., modified iPSC) may also include a modification to increase expression of one or more tolerogenic factors. In some embodiments, the tolerogenic factor is one or more of DUX4, B2M-HLA-E, CD16, CD52, CD47, CD27, CD200, HLA-C, HLA-E, HLA-E heavy chain, HLA-G, PD-L1, IDO1, CTLA4-Ig, Cl-Inhibitor, IL-10, IL-35, FASL, CCL21, MFGE8, SERPINB9, CD35, IL-39, CD16 Fc Receptor, IL15-RF, and H2-M3, or any combination thereof. In some embodiments, the one or more tolerogenic factors are selected from the group consisting of CD16, CD24, CD35, CD39, CD46, CD47, CD52, CD55, CD59, CD64, CD200, CCL22, CTLA4-Ig, C1 inhibitor, FASL, IDO1, HLA-C, HLA-E, HLA-E heavy chain, HLA-G, IL-10, IL-35, PD-L1, SERPINB9, CCL21, MFGE8, DUX4, B2M-HLA-E, CD27, IL-39, CD16 Fc Receptor, IL15-RF, H2-M3 (HLA-G), A20/TNFAIP3, CR1, HLA-F, and MANF. In some embodiments, the modification to increase expression of one or more tolerogenic factors is or includes increased expression of CD47. In some embodiments, the modification to increase expression of one or more tolerogenic factors is or includes increased expression of PD-L1. In some embodiments, the modification to increase expression of one or more tolerogenic factors is or includes increased expression of HLA-E. In some embodiments, the modification to increase expression of one or more tolerogenic factors is or includes increased expression of HLA-G. In some embodiments, the modification to increase expression of one or more tolerogenic factors is or includes increased expression of CCL21, PD-L1, FasL, Serpinb9, H2-M3 (HLA-G), CD47, CD200, and Mfge8.

In some embodiments, the modified pluripotent stem cells (e.g., modified iPSC) cells include one or more genomic modifications that reduce expression of MHC class I molecules and a modification that increases expression of CD47. In other words, the modified pluripotent stem cells comprise exogenous CD47 proteins and exhibit reduced or silenced surface expression of one or more MHC class I molecules. In some embodiments, the cells include one or more genomic modifications that reduce expression of MHC class II molecules and a modification that increases expression of CD47. In some instances, the modified cells comprise exogenous CD47 nucleic acids and proteins, and exhibit reduced or silenced surface expression of one or more MHC class I molecules. In some embodiments, the cells include one or more genomic modifications that reduce or eliminate expression of MHC class II molecules, one or more genomic modifications that reduce or eliminate expression of MHC class II molecules, and a modification that increases expression of CD47. In some embodiments, the modified pluripotent stem cells comprise exogenous CD47 proteins, exhibit reduced or silenced surface expression of one or more MHC class I molecules and exhibit reduced or lack surface expression of one or more MHC class II molecules. In many embodiments, the cells are B2M indel/indel, CIITAindel/indel, CD47tg cells.

In certain embodiments, the modified pluripotent stem cells may comprise a modification that modulates or regulates the expression of CD142. In some embodiments, the modification reduces or eliminates expression of CD142. In some embodiments, the modification that reduces expression of CD142 reduces CD142 protein expression. In some embodiments, the modification eliminates CD142 gene activity. In some embodiments, the modification comprises inactivation or disruption of both alleles of the CD142 gene. In some embodiments, the modification comprises inactivation or disruption of all CD142 coding sequences in the cell. In some embodiments, the inactivation or disruption comprises an indel in the CD142 gene. In some embodiments, the modification is a frameshift mutation or a deletion of a contiguous stretch of genomic DNA of the CD142 gene. In some embodiments, the CD142 gene is knocked out.

In some embodiments, the provided modified pluripotent stem cells (e.g., modified iPSC) cells may also contain one or more modifications that increase expression of one or more complement inhibitors selected from the group consisting of CD46, CD59, CD55, CD35 and combinations thereof. In some embodiments, the modification(s) that increase expression comprise increased surface expression, and/or the modifications that reduce expression comprise reduced surface expression. In some embodiments, the modification(s) that increase expression of the one or more complement inhibitor comprises an exogenous polynucleotide encoding CD46, an exogenous polynucleotide encoding CD59, an exogenous polynucleotide encoding CD55 and/or an exogenous polynucleotide encoding CD35. In some embodiments, the one or more complement inhibitor is CD46 and CD59, optionally wherein the modification comprises an exogenous polynucleotide encoding CD46 and an exogenous polynucleotide encoding CD59.the one or more complement inhibitor is CD46, CD59 and CD55, optionally wherein the modification comprises an exogenous polynucleotide encoding CD46, an exogenous polynucleotide encoding CD59 and an exogenous polynucleotide encoding CD55. In some embodiments, the modified cell comprises a multicistronic vector comprising two or more exogenous polypeptides selected from the group consisting of one or more exogenous polynucleotide encoding the one or more tolerogenic factors, an exogenous polynucleotide encoding CD46, an exogenous polynucleotide encoding CD59, and an exogenous polynucleotide encoding CD55 polypeptide. In some embodiments, each of the polynucleotides are separated by an IRES or a self-cleaving peptide.

In some embodiments, modulation of expression of the one or more target immune molecules, e.g. tolerogenic factor (e.g., increased expression), and the modulation of expression of the MHC class I molecules and/or MHC class II molecules (e.g., reduced or eliminated expression), is relative to the amount of expression of said molecule(s) in a pluripotent stem cell that does not comprise the modification(s) (i.e., unmodified pluripotent stem cell). In some embodiments, the cells are engineered or modified to have reduced or increased expression of one or more targets relative to an unaltered or unmodified wild-type cell. In some embodiments, the cells are engineered or modified to have constitutive reduced or increased expression of one or more targets relative to an unaltered or unmodified cell. In some embodiments, the cells are engineered or modified to have regulatable reduced or increased expression of one or more targets relative to an unaltered or unmodified cell. In some embodiments, the cells comprise increased expression of a tolerogenic factor (e.g., CD47) and reduced expression of the MHC class I molecules and/or MHC class II molecules relative to a wild-type cell or a control cell of the same cell type. Examples of wild type or control cells include pluripotent cells (e.g., embryonic stem cells or iPSCs). However, by way of example, in the context of an engineered cell, as used herein, “wild-type” or “control” can also mean an engineered cell that may contain nucleic acid changes resulting in reduced expression of MHC I and/or II, but did not undergo the gene editing procedures to result in overexpression of CD47 proteins. In the context of an iPSC or a progeny thereof, “wild-type” or “control” also means an iPSC or progeny thereof that may contain nucleic acid changes resulting in pluripotency but did not undergo the gene editing procedures of the present disclosure to achieve reduced expression of MHC I and/or II, and/or overexpression of CD47 proteins. In some embodiments, the wild-type cell or the control cell is a starting material. In some embodiments, an iPSC cell line starting material is a starting material that is considered a wild-type or control cell as contemplated herein. In some embodiments, the starting material is otherwise modified or engineered to have altered expression of one or more genes to generate the engineered cell. Hence, it is understood that reference to an “unmodified cell” can be a control cell that has been engineered in some aspects but does not contain all of the modifications by the gene editing procedures of the present disclosure to achieve reduced expression of MHC I and/or II, and/or overexpression of a tolerogenic protein (e.g., CD47).

In some embodiments, the unmodified cell or wildtype cell expresses the tolerogenic factor, the MHC class I molecules, and/or the MHC class II molecules. In some embodiments, the unmodified cell or wildtype cell does not express the one or more tolerogenic factors, the MHC class I molecules, and/or the MHC class II molecules. In some embodiments wherein the unmodified cell or wildtype cell does not express the tolerogenic factor is used to generate the engineered primary cell, the provided engineered primary cells include a modification to overexpress the one or more tolerogenic factors or increase the expression of the one or more tolerogenic factors from 0%. It is understood that if the cell prior to the engineering does not express a detectable amount of the tolerogenic factor, then a modification that results in any detectable amount of an expression of the tolerogenic factor is an increase in the expression compared to the similar cell that does not contain the modifications.

In some embodiments, the population of modified pluripotent stem cells described elicits a reduced level of immune activation or no immune activation upon administration to a recipient subject. In some embodiments, the cells elicit a reduced level of systemic TH1 activation or no systemic TH1 activation in a recipient subject. In some embodiments, the cells elicit a reduced level of immune activation of peripheral blood mononuclear cells (PBMCs) or no immune activation of PBMCs in a recipient subject. In some embodiments, the cells elicit a reduced level of donor-specific IgG antibodies or no donor specific IgG antibodies against the cells upon administration to a recipient subject. In some embodiments, the cells elicit a reduced level of IgM and IgG antibody production or no IgM and IgG antibody production against the cells in a recipient subject. In some embodiments, the cells elicit a reduced level of cytotoxic T cell killing of the cells upon administration to a recipient subject.

In some embodiments, the modified pluripotent stem cells provided herein comprise a “suicide gene” or “suicide switch.” A suicide gene or suicide switch can be incorporated to function as a “safety switch” that can cause the death of the cell, such as after the modified pluripotent stem cells cell is administered to a subject and if the cells should grow and divide in an undesired manner. The “suicide gene” ablation approach includes a suicide gene in a gene transfer vector encoding a protein that results in cell killing only when activated by a specific compound. A suicide gene may encode an enzyme that selectively converts a nontoxic compound into highly toxic metabolites. The result is specifically eliminating cells expressing the enzyme. In some embodiments, the suicide gene is the herpesvirus thymidine kinase (HSV-tk) gene and the trigger is ganciclovir. In other embodiments, the suicide gene is a cytosine deaminase (e.g., the Escherichia coli cytosine deaminase (EC-CD)) gene and the trigger is 5-fluorocytosine (5-FC) (Barese et al, Mol. Therap. 20 (10): 1932-1943 (2012), Xu et al, Cell Res. 8:73-8 (1998), both incorporated herein by reference in their entirety).

In some aspects, provided are modified pluripotent stem cell having (1) reduced expression of MHC I and/or MHC II; and (2) a transgene comprising CD47 and a safety switch inserted at a safe harbor locus, wherein the safe harbor locus is selected from the group consisting of an AAVS1, ABO, CCR5, CLYBL, CXCR4, F3, FUT1, HMGB1, KDM5D, LRP1, MICA, MICB, RHD, ROSA26, and SHS231 locus. In some aspects, provided are modified pluripotent stem cells having (1) reduced expression of MHC I and/or MHC II; and (2) a transgene comprising CD47 and HSVtk flanked by CLYBL homology arms, wherein the transgene is inserted at the CLYBL locus. In some embodiments, the modified pluripotent stem cell has B2M and/or CIITA knockout. In some embodiments, the B2M and/or CIITA knockout occur in both alleles.

In other embodiments, the suicide gene is an inducible Caspase protein. An inducible Caspase protein comprises at least a portion of a Caspase protein capable of inducing apoptosis. In preferred embodiments, the inducible Caspase protein is iCasp9. It comprises the sequence of the human FK506-binding protein, FKBP12, with an F36V mutation, connected through a series of amino acids to the gene encoding human caspase 9. FKBP12-F36V binds with high affinity to a small-molecule dimerizing agent, API 903. Thus, the suicide function of iCasp9 in the instant invention is triggered by the administration of a chemical inducer of dimerization (CID). In some embodiments, the CID is the small molecule drug API 903. Dimerization causes the rapid induction of apoptosis. (See WO2011146862; Stasi et al, N. Engl. J. Med 365; 18 (2011); Tey et al, Biol. Blood Marrow Transplant. 13:913-924 (2007), each of which are incorporated by reference herein in their entirety.)

Inclusion of a safety switch or suicide gene allows for controlled killing of the cells in the event of cytotoxicity or other negative consequences to the recipient, thus increasing the safety of cell-based therapies, including those using tolerogenic factors.

In some embodiments, a safety switch can be incorporated into, such as introduced, into the modified pluripotent stem cells provided herein to provide the ability to induce death or apoptosis of modified cells containing the safety switch, for example if the cells grow and divide in an undesired manner or cause excessive toxicity to the host. Thus, the use of safety switches enables one to conditionally eliminate aberrant cells in vivo and can be a critical step for the application of cell therapies in the clinic. Safety switches and their uses thereof are described in, for example, Duzgune §, Origins of Suicide Gene Therapy (2019); Duzgune § (eds), Suicide Gene Therapy. Methods in Molecular Biology, vol. 1895 (Humana Press, New York, NY) (for HSV-tk, cytosine deaminase, nitroreductase, purine nucleoside phosphorylase, and horseradish peroxidase); Zhou and Brenner, Exp Hematol 44 (11): 1013-1019 (2016) (for iCaspase9); Wang et al., Blood 18 (5): 1255-1263 (2001) (for huEGFR); U.S. Patent Application Publication No. 20180002397 (for HER1); and Philip et al., Blood124 (8): 1277-1287 (2014) (for RQR8).

In some embodiments, the safety switch can cause cell death in a controlled manner, for example, in the presence of a drug or prodrug or upon activation by a selective exogenous compound. In some embodiments, the safety switch is selected from the group consisting of herpes simplex virus thymidine kinase (HSV-tk), cytosine deaminase (CyD), nitroreductase (NTR), purine nucleoside phosphorylase (PNP), horseradish peroxidase, inducible caspase 9 (iCasp9), rapamycin-activated caspase 9 (rapaCasp9), CCR4, CD16, CD19, CD20, CD30, EGFR, GD2, HER1, HER2, MUC1, PSMA, and RQR8.

In some embodiments, the safety switch may be a transgene encoding a product with cell killing capabilities when activated by a drug or prodrug, for example, by turning a non-toxic prodrug to a toxic metabolite inside the cell. In these embodiments, cell killing is activated by contacting a modified cell with the drug or prodrug. In some cases, the safety switch is HSV-tk, which converts ganciclovir (GCV) to GCV-triphosphate, thereby interfering with DNA synthesis and killing dividing cells. In some cases, the safety switch is CyD or a variant thereof, which converts the antifungal drug 5-fluorocytosine (5-FC) to cytotoxic 5-fluorouracil (5-FU) by catalyzing the hydrolytic deamination of cytosine into uracil. 5-FU is further converted to potent anti-metabolites (5-FdUMP, 5-FdUTP, 5-FUTP) by cellular enzymes. These compounds inhibit thymidylate synthase and the production of RNA and DNA, resulting in cell death. In some cases, the safety switch is NTR or a variant thereof, which can act on the prodrug CB 1954 via reduction of the nitro groups to reactive N-hydroxylamine intermediates that are toxic in proliferating and nonproliferating cells. In some cases, the safety switch is PNP or a variant thereof, which can turn prodrug 6-methylpurine deoxyriboside or fludarabine into toxic metabolites to both proliferating and nonproliferating cells. In some cases, the safety switch is horseradish peroxidase or a variant thereof, which can catalyze indole-3-acetic acid (IAA) to a potent cytotoxin and thus achieve cell killing.

In some embodiments, the safety switch may be an iCasp9. Caspase 9 is a component of the intrinsic mitochondrial apoptotic pathway which, under physiological conditions, is activated by the release of cytochrome C from damaged mitochondria. Activated caspase 9 then activates caspase 3, which triggers terminal effector molecules leading to apoptosis. The iCasp9 may be generated by fusing a truncated caspase 9 (without its physiological dimerization domain or caspase activation domain) to a FK506 binding protein (FKBP), FKBP12-F36V, via a peptide linker. The iCasp9 has low dimer-independent basal activity and can be stably expressed in host cells (e.g., human T cells) without impairing their phenotype, function, or antigen specificity. However, in the presence of chemical inducer of dimerization (CID), such as rimiducid (AP1903), AP20187, and rapamycin, iCasp9 can undergo inducible dimerization and activate the downstream caspase molecules, resulting in apoptosis of cells expressing the iCasp9. See, e.g., PCT Application Publication No. WO2011/146862; Stasi et al., N. Engl. J. Med. 365; 18 (2011); Tey et al., Biol. Blood Marrow Transplant 13:913-924 (2007). In particular, the rapamycininducible caspase 9 variant is called rapaCasp9. See Stavrou et al., Mal. Ther. 26 (5): 1266-1276 (2018). Thus, iCasp9 can be used as a safety switch to achieve controlled killing of the host cells.

In some embodiments, the safety switch may be a membrane-expressed protein which allows for cell depletion after administration of a specific antibody to that protein. Safety switches of this category may include, for example, one or more transgene encoding CCR4, CD16, CD19, CD20, CD30, EGFR, GD2, HER1, HER2, MUC1, PSMA, or RQR8 for surface expression thereof. These proteins may have surface epitopes that can be targeted by specific antibodies. In some embodiments, the safety switch comprises CCR4, which can be recognized by an anti-CCR4 antibody. Non-limiting examples of suitable anti-CCR4 antibodies include mogamulizumab and biosimilars thereof. In some embodiments, the safety switch comprises CD16 or CD30, which can be recognized by an anti-CD16 or anti-CD30 antibody. Non-limiting examples of such antiCD16 or anti-CD30 antibody include AFM13 and biosimilars thereof. In some embodiments, the safety switch comprises CD19, which can be recognized by an anti-CD19 antibody. Non-limiting examples of such anti-CD19 antibody include MOR208 and biosimilars thereof. In some embodiments, the safety switch comprises CD20, which can be recognized by an anti-CD20 antibody. Non-limiting examples of such anti-CD20 antibody include obinutuzumab, ublituximab, ocaratuzumab, rituximab, rituximab-Rllb, and biosimilars thereof. Cells that express the safety switch are thus CD20-positive and can be targeted for killing through administration of an anti-CD20 antibody as described. In some embodiments, the safety switch comprises EGFR, which can be recognized by an anti-EGFR antibody. Non-limiting examples of such anti-EGFR antibody include tomuzotuximab, RO5083945 (GA201), cetuximab, and biosimilars thereof. In some embodiments, the safety switch comprises GD2, which can be recognized by an anti-GD2 antibody. Non-limiting examples of such anti-GD2 antibody include Hul4.18K322A, Hul4.18-IL2, Hu3F8, dinituximab, c.60C3-Rllc, and biosimilars thereof.

In some embodiments, the safety switch may be an exogenously administered agent that recognizes one or more tolerogenic factors on the surface of the modified cell. In some embodiments, the exogenously administered agent is an antibody directed against or specific to a tolerogenic agent, e.g., an anti-CD47 antibody. By recognizing and blocking a tolerogenic factor on the modified cell, an exogenously administered antibody may block the immune inhibitory functions of the tolerogenic factor thereby re-sensitizing the immune system to the modified cells. For instance, for a modified cell that overexpresses CD47 an exogenously administered anti-CD47 antibody may be administered to the subject, resulting in masking of CD47 on the modified cell and triggering of an immune response to the modified pluripotent stem cells.

In some embodiments, the safety switch can include any of the strategies as described in WO2021146627A1, which is incorporated by reference in its entirety.

In some embodiments, the method further comprises introducing an expression vector comprising an inducible suicide switch into the cell.

In some embodiments, the modified pluripotent stem cells are derived from a source cell already comprising one or more of the desired modifications. In some embodiments, in view of the teachings provided herein one of ordinary skill in the art will readily appreciate how to assess what modifications are required to arrive at the desired final form of a modified pluripotent stem cells, and that not all reduced or increased levels of target components are achieved via active engineering. In some embodiments, the modifications of the modified cell may be in any order, and not necessarily the order listed in the descriptive language provided herein.

In some embodiments, provided herein is a method of generating a modified pluripotent stem cell, comprising: (a) reducing or eliminating the expression of MHC class I and/or MHC class II human leukocyte antigens in the cell; and (b) increasing the expression of a tolerogenic factor in the cell. In some embodiments, the one or more tolerogenic factors is selected from DUX4, B2M-HLA-E, CD16, CD52, CD47, CD27, CD200, HLA-C, HLA-E, HLA-E heavy chain, HLA-G, PD-L1, IDO1, CTLA4-Ig, Cl-Inhibitor, IL-10, IL-35, FASL, CCL21, MFGE8, SERPINB9, CD35, IL-39, CD16 Fc Receptor, IL15-RF, and H2-M3. In some embodiments, the one or more tolerogenic factors are selected from the group consisting of CD16, CD24, CD35, CD39, CD46, CD47, CD52, CD55, CD59, CD64, CD200, CCL22, CTLA4-Ig, C1 inhibitor, FASL, IDO1, HLA-C, HLA-E, HLA-E heavy chain, HLA-G, IL-10, IL-35, PD-L1, SERPINB9, CCL21, MFGE8, DUX4, B2M-HLA-E, CD27, IL-39, CD16 Fc Receptor, IL15-RF, H2-M3 (HLA-G), A20/TNFAIP3, CR1, HLA-F, and MANF. In some embodiments, the one or more tolerogenic factors is CD47. In some embodiments, the method comprises reducing or eliminating the expression of MHC class I and MHC class II human leukocyte antigens. In some embodiments, the reducing or increasing expression comprise performing one or more modifications to the cell using a guided nuclease (e.g., a CRISPR/Cas system). In some embodiments, the method further comprises introducing an expression vector comprising an inducible suicide switch into the cell. In some embodiments, the method further comprises increasing the expression of one or more complement inhibitors selected from the group consisting of CD46, CD59, and CD55 in said cell.

In some embodiments, provided herein is a method of generating a modified pluripotent stem cells cell, comprising: (a) increasing the expression of CCL21, PD-L1, FASL, SERPINB9, HLA-G, CD47, CD200, and MFGE8 in the cell, and (b) reducing expression of CD142 in the cell. In some embodiments, the reducing or increasing expression comprise performing one or more modifications to the cell using a guided nuclease (e.g., a CRISPR/Cas system). In some embodiments, the method further comprises introducing an expression vector comprising an inducible suicide switch into the cell. In some embodiments, the method further comprises increasing the expression of one or more complement inhibitors selected from the group consisting of CD46, CD59, and CD55 in said cell.

Once the modified iPSCs cells have been generated, they may be assayed for their hypoimmunogenicity and/or retention of pluripotency as is described in WO2016183041 and WO2018132783. In some embodiments, hypoimmunogenicity is assayed using a number of techniques as exemplified in FIG. 13 and FIG. 15 of WO2018132783. These techniques include transplantation into allogeneic hosts and monitoring for hypoimmunogenic pluripotent cell growth (e.g., teratomas) that escape the host immune system. In some instances, hypoimmunogenic pluripotent cell derivatives are transduced to express luciferase and can then followed using bioluminescence imaging. Similarly, the T cell and/or B cell response of the host animal to such cells are tested to confirm that the cells do not cause an immune reaction in the host animal. T cell responses can be assessed by Elispot, ELISA, FACS, PCR, or mass cytometry (CYTOF). B cell responses or antibody responses are assessed using FACS or Luminex. Additionally or alternatively, the cells may be assayed for their ability to avoid innate immune responses, e.g., NK cell killing, as is generally shown in FIGS. 14 and 15 of WO2018132783.

In some embodiments, the immunogenicity of the cells is evaluated using T cell immunoassays such as T cell proliferation assays, T cell activation assays, and T cell killing assays recognized by those skilled in the art. In some cases, the T cell proliferation assay includes pretreating the cells with interferon-gamma and coculturing the cells with labelled T cells and assaying the presence of the T cell population (or the proliferating T cell population) after a preselected amount of time. In some cases, the T cell activation assay includes coculturing T cells with the cells outlined herein and determining the expression levels of T cell activation markers in the T cells.

In vivo assays can be performed to assess the immunogenicity of the cells outlined herein. In some embodiments, the survival and immunogenicity of modified iPSCs or modified SC-beta cells is determined using an allogeneic humanized immunodeficient mouse model. In some instances, the modified iPSCs are transplanted into an allogeneic humanized NSG-SGM3 mouse and assayed for cell rejection, cell survival, and teratoma formation. In some instances, grafted modified iPSCs or differentiated cells thereof display long-term survival in the mouse model.

Additional techniques for determining immunogenicity including hypoimmunogenicity of the cells are described in, for example, Deuse et al., Nature Biotechnology, 2019, 37, 252-258 and Han et al., Proc Natl Acad Sci USA, 2019, 116 (21), 10441-10446, the disclosures including the figures, figure legends, and description of methods are incorporated herein by reference in their entirety.

Similarly, the retention of pluripotency may be tested in a number of ways. In one embodiment, pluripotency is assayed by the expression of certain pluripotency-specific factors as generally described herein and shown in FIG. 29 of WO2018132783. Additionally or alternatively, the pluripotent cells are differentiated into one or more cell types as an indication of pluripotency.

Once the modified pluripotent stem cells (modified iPSCs) have been generated, they can be maintained in an undifferentiated state as is known for maintaining iPSCs. For example, the cells can be cultured on Matrigel using culture media that prevents differentiation and maintains pluripotency. In addition, they can be in culture medium under conditions to maintain pluripotency.

Once altered, the presence of expression of any of the molecule described herein can be assayed using known techniques, such as Western blots, ELISA assays, FACS assays, and the like.

### 1. Inactivation or Disruption of Target Genes

a. Target Genes
1) MHC Class I and/or MHC Class II

In some embodiments, the provided modified pluripotent stem cells comprise a modification (e.g., genetic modifications) of one or more target polynucleotide or protein sequences (also interchangeably referred to as a target gene) that regulate (e.g., reduce or eliminate) the expression of either MHC class I molecules, MHC class II molecules, or MHC class I and MHC class II molecules. In some embodiments, the cell to be modified is an unmodified cell that has not previously been introduced with the one or more modifications. In some embodiments, a genetic editing system is used to modify one or more target polynucleotide sequences that regulate (e.g., reduce or eliminate) the expression of either MHC class I molecules, MHC class II molecules, or MHC class I and MHC class II molecules. In certain embodiments, the genome of the cell has been altered to reduce or delete components required or involved in facilitating HLA expression, such as expression of MHC class I and/or MHC class II molecules on the surface of the cell. For instance, in some embodiments, expression of a beta-2-microgloublin (B2M), a component of MHC class I molecules, is reduced or eliminated in the cell, thereby reducing or elimination the protein expression (e.g., cell surface expression) of MHC class I by the modified pluripotent stem cells.

In some embodiments, any of the described modifications in the modified pluripotent stem cells that regulate (e.g., reduce or eliminate) expression of one or more target polynucleotide or protein in the modified pluripotent stem cells may be combined with one or more modifications to overexpress a polynucleotide (e.g., tolerogenic factor, such as CD47).

In some embodiments, reduction of MHC class I and/or MHC class II expression can be accomplished, for example, by one or more of the following: (1) directly targeting the MHC class I genes such as the polymorphic HLA alleles (HLA-A, HLA-B, HLA-C) and/or the MHC class II genes such as HLA-DP, HLA-DQ, and/or HLA-DR; (2) removal of B2M, which will reduce surface trafficking of all MHC class I molecules; and/or (3) deletion of one or more components of the MHC enhanceosomes, such as LRC5, RFX-5, RFXANK, RFXAP, IRF1, NF-Y (including NFY-A, NFY-B, NFY-C), and CIITA that are critical for HLA expression. In some embodiments, reduction of MHC class II also may be accomplished by reducing expression, such as by knocking out the gene encoding CD74 in a cell, which is involved in the formation and transport of MHC class II.

In certain embodiments, HLA expression is interfered with. In some embodiments, HLA expression is interfered with by targeting individual HLAs (e.g., knocking out expression of one or more HLA class I molecules such as HLA-A, HLA-B and/or HLA-C and/or knocking out expression of one or more HLA class I molecules such as HLA-DP, HLA-DQ, and/or HLA-DR), targeting transcriptional regulators of HLA expression (e.g., knocking out expression of NLRC5, CIITA, RFX5, RFXAP, RFXANK, NFY-A, NFY-B, NFY-C and/or IRF-1), blocking surface trafficking of MHC class I molecules (e.g., knocking out expression of B2M and/or TAP1), and/or targeting with HLA-Razor (see, e.g., WO2016183041). In some embodiments, reduction of HLA class II also may be accomplished by reducing expression, such as by knocking out, the gene encoding CD74 in a human cell, which is involved in the formation and transport of HLA class II molecules.

In certain aspects, the modified pluripotent stem cells disclosed herein do not express one or more human leukocyte antigens corresponding to MHC class I (e.g., HLA-A, HLA-B and/or HLA-C) and/or MHC class II (e.g., HLA-DP, HLA-DQ, and/or HLA-DR) and are thus characterized as being hypoimmunogenic. For example, in certain aspects, the modified pluripotent stem cells disclosed herein have been modified such that the cells, including any stem cell or a differentiated stem cell prepared therefrom, do not express or exhibit reduced expression of one or more of the following MHC class I molecules: HLA-A, HLA-B and HLA-C. In some embodiments, one or more of HLA-A, HLA-B and HLA-C may be “knocked-out” of a cell. A cell that has a knocked-out HLA-A gene, HLA-B gene, and/or HLA-C gene may exhibit reduced or eliminated expression of each knocked-out gene. In some aspects, the modified pluripotent stem cells disclosed herein have been modified such that the cells, including any stem cell or a differentiated stem cell prepared therefrom, do not express or exhibit reduced expression of one or more of the following MHC class II molecules: HLA-DP, HLA-DQ, and HLA-DR. In some embodiments, one or more of HLA-DP, HLA-DQ, and HLA-DR may be “knocked-out” of a cell. A cell that has a knocked-out HLA-DP gene, HLA-DQ gene and/or HLA-DR gene may exhibit reduced or eliminated expression of each knocked-out gene.

In certain embodiments, the expression of MHC class I molecules and/or MHC class II molecules is modulated by targeting and deleting a contiguous stretch of genomic DNA, thereby reducing or eliminating expression of a target gene selected from the group consisting of B2M, CIITA, and NLRC5. In some embodiments, MHC class I molecules can alternatively or additionally be modulated by reducing or eliminating expression of TAP1. In some embodiments, MHC class II molecules can alternatively or additionally be modulated by reducing or eliminating expression of CD74.

In some embodiments, the provided modified pluripotent stem cells comprise a modification of one or more target polynucleotide sequence that regulate MHC class I. Exemplary methods for reducing expression of MHC class I are described in sections below. In some embodiments, the targeted polynucleotide sequence is one or both of B2M and NLRC5. In some embodiments, the cell comprises a genetic editing modification (e.g., an indel) to the B2M gene. In some embodiments, the cell comprises a genetic editing modification (e.g., an indel) to the NLRC5 gene. In some embodiments, the cell comprises a genetic editing modification (e.g., an indel) to the TAP1 gene. In some embodiments, the cell comprises genetic editing modifications (e.g., indels) to the B2M and CIITA genes.

In some embodiments, a modification that reduces expression of an MHC class I molecule is a modification that reduces expression of B2M. In some embodiments, the modification that reduces B2M expression reduces B2M mRNA expression. In some embodiments, the reduced mRNA expression of B2M is relative to an unmodified or wild-type cell of the same cell type that does not comprise the modification. In some embodiments, the mRNA expression of B2M is reduced by more than about 5%, such as reduced by more than about any of 10%, 20%, 30%, 40%, 50%, 60%, 70%, 80%, 90%, or more. In some embodiments, the mRNA expression of B2M is reduced by up to about 100%, such as reduced by up to about any of 90%, 80%, 70%, 60%, 50%, 40%, 30%, 20%, 10%, 5%, or less. In some embodiments, the mRNA expression of B2M is reduced by any of about 5%, 10%, 20%, 30%, 40%, 50%, 60%, 70%, 80%, 90%, or 100%. In some embodiments, the mRNA expression of B2M is eliminated (e.g., 0% expression of B2M mRNA). In some embodiments, the modification that reduces B2M mRNA expression eliminates B2M gene activity.

In some embodiments, the modification that reduces B2M expression reduces B2M protein expression. In some embodiments, the reduced protein expression of B2M is relative to an unmodified or wild-type cell of the same cell type that does not comprise the modification. In some embodiments, the protein expression of B2M is reduced by more than about 5%, such as reduced by more than about any of 10%, 20%, 30%, 40%, 50%, 60%, 70%, 80%, 90%, or more. In some embodiments, the protein expression of B2M is reduced by up to about 100%, such as reduced by up to about any of 90%, 80%, 70%, 60%, 50%, 40%, 30%, 20%, 10%, 5%, or less. In some embodiments, the protein expression of B2M is reduced by any of about 5%, 10%, 20%, 30%, 40%, 50%, 60%, 70%, 80%, 90%, or 100%. In some embodiments, the protein expression of B2M is eliminated (e.g., no detectable expression of B2M protein). In some embodiments, the modification that reduces B2M protein expression eliminates B2M gene activity.

In some embodiments, the modification that reduces B2M expression comprises inactivation or disruption of the B2M gene. In some embodiments, the modification that reduces B2M expression comprises inactivation or disruption of one allele of the B2M gene. In some embodiments, the modification that reduces B2M expression comprises inactivation or disruption comprises inactivation or disruption of both alleles of the B2M gene.

In some embodiments, the modification comprises inactivation or disruption of one or more B2M coding sequences in the cell. In some embodiments, the modification comprises inactivation or disruption of all B2M coding sequences in the cell. In some embodiments, the modification comprises inactivation or disruption comprises an indel in the B2M gene. In some embodiments, the modification is a frameshift mutation of genomic DNA of the B2M gene. In some embodiments, the modification is a deletion of genomic DNA of the B2M gene. In some embodiments, the modification is a deletion of a contiguous stretch of genomic DNA of the B2M gene. In some embodiments, the B2M gene is knocked out.

In some embodiments, a modification that reduces expression of an MHC class I molecule is a modification that reduces expression of NLRC5. In some embodiments, decreased or eliminated expression of NLRC5 reduces or eliminates expression of one or more of the following MHC I molecules—HLA-A, HLA-B, and HLA-C. In some embodiments, the modification that reduces NLRC5 expression reduces NLRC5 mRNA expression. In some embodiments, the reduced mRNA expression of NLRC5 is relative to an unmodified or wild-type cell of the same cell type that does not comprise the modification. In some embodiments, the mRNA expression of NLRC5 is reduced by more than about 5%, such as reduced by more than about any of 10%, 20%, 30%, 40%, 50%, 60%, 70%, 80%, 90%, or more. In some embodiments, the mRNA expression of NLRC5 is reduced by up to about 100%, such as reduced by up to about any of 90%, 80%, 70%, 60%, 50%, 40%, 30%, 20%, 10%, 5%, or less. In some embodiments, the mRNA expression of NLRC5 is reduced by any of about 5%, 10%, 20%, 30%, 40%, 50%, 60%, 70%, 80%, 90%, or 100%. In some embodiments, the mRNA expression of NLRC5 is eliminated (e.g., 0% expression of NLRC5 mRNA). In some embodiments, the modification that reduces NLRC5 mRNA expression eliminates NLRC5 gene activity.

In some embodiments, the modification that reduces NLRC5 expression reduces NLRC5 protein expression. In some embodiments, the reduced protein expression of NLRC5 is relative to an unmodified or wild-type cell of the same cell type that does not comprise the modification. In some embodiments, the protein expression of NLRC5 is reduced by more than about 5%, such as reduced by more than about any of 10%, 20%, 30%, 40%, 50%, 60%, 70%, 80%, 90%, or more. In some embodiments, the protein expression of NLRC5 is reduced by up to about 100%, such as reduced by up to about any of 90%, 80%, 70%, 60%, 50%, 40%, 30%, 20%, 10%, 5%, or less. In some embodiments, the protein expression of NLRC5 is reduced by any of about 5%, 10%, 20%, 30%, 40%, 50%, 60%, 70%, 80%, 90%, or 100%. In some embodiments, the protein expression of NLRC5 is eliminated (e.g., no detectable expression of NLRC5 protein). In some embodiments, the modification that reduces NLRC5 protein expression eliminates NLRC5 gene activity.

In some embodiments, the modification that reduces NLRC5 expression comprises inactivation or disruption of the NLRC5 gene. In some embodiments, the modification that reduces NLCR5 expression comprises inactivation or disruption of one allele of the NLRC5 gene. In some embodiments, the modification that reduces NLRC5 expression comprises inactivation or disruption comprises inactivation or disruption of both alleles of the NLRC5 gene.

In some embodiments, the modification comprises inactivation or disruption of one or more NLRC5 coding sequences in the cell. In some embodiments, the modification comprises inactivation or disruption of all NLRC5 coding sequences in the cell. In some embodiments, the modification comprises inactivation or disruption comprises an indel in the NLRC5 gene. In some embodiments, the modification is a frameshift mutation of genomic DNA of the NLRC5 gene. In some embodiments, the modification is a deletion of genomic DNA of the NLRC5 gene. In some embodiments, the modification is a deletion of a contiguous stretch of genomic DNA of the NLRC5 gene. In some embodiments, the NLRC5 gene is knocked out.

In some embodiments, a modification that reduces expression of an MHC class I molecule is a modification that reduces expression of TAP1. In some embodiments, decreased or eliminated expression of TAP1 reduces or eliminates expression of one or more of the following MHC I molecules-HLA-A, HLA-B, and HLA-C. In some embodiments, the modification that reduces TAP1 expression reduces TAP1 mRNA expression. In some embodiments, the reduced mRNA expression of TAP1 is relative to an unmodified or wild-type cell of the same cell type that does not comprise the modification. In some embodiments, the mRNA expression of TAP1 is reduced by more than about 5%, such as reduced by more than about any of 10%, 20%, 30%, 40%, 50%, 60%, 70%, 80%, 90%, or more. In some embodiments, the mRNA expression of TAP1 is reduced by up to about 100%, such as reduced by up to about any of 90%, 80%, 70%, 60%, 50%, 40%, 30%, 20%, 10%, 5%, or less. In some embodiments, the mRNA expression of TAP1 is reduced by any of about 5%, 10%, 20%, 30%, 40%, 50%, 60%, 70%, 80%, 90%, or 100%. In some embodiments, the mRNA expression of TAP1 is eliminated (e.g., 0% expression of TAP1 mRNA). In some embodiments, the modification that reduces TAP1 mRNA expression eliminates TAP1 gene activity.

In some embodiments, the modification that reduces TAP1 expression reduces TAP1 protein expression. In some embodiments, the reduced protein expression of TAP1 is relative to an unmodified or wild-type cell of the same cell type that does not comprise the modification. In some embodiments, the protein expression of TAP1 is reduced by more than about 5%, such as reduced by more than about any of 10%, 20%, 30%, 40%, 50%, 60%, 70%, 80%, 90%, or more. In some embodiments, the protein expression of TAP1 is reduced by up to about 100%, such as reduced by up to about any of 90%, 80%, 70%, 60%, 50%, 40%, 30%, 20%, 10%, 5%, or less. In some embodiments, the protein expression of TAP1 is reduced by any of about 5%, 10%, 20%, 30%, 40%, 50%, 60%, 70%, 80%, 90%, or 100%. In some embodiments, the protein expression of TAP1 is eliminated (e.g., no detectable expression of TAP1 protein). In some embodiments, the modification that reduces TAP1 protein expression eliminates TAP1 gene activity.

In some embodiments, the modification that reduces TAP1 expression comprises inactivation or disruption of the TAP1 gene. In some embodiments, the modification that reduces TAP1 expression comprises inactivation or disruption of one allele of the TAP1 gene. In some embodiments, the modification that reduces TAP1 expression comprises inactivation or disruption comprises inactivation or disruption of both alleles of the TAP1 gene.

In some embodiments, the modification comprises inactivation or disruption of one or more TAP1 coding sequences in the cell. In some embodiments, the modification comprises inactivation or disruption of all TAP1 coding sequences in the cell. In some embodiments, the modification comprises inactivation or disruption comprises an indel in the TAP1 gene. In some embodiments, the modification is a frameshift mutation of genomic DNA of the TAP1 gene. In some embodiments, the modification is a deletion of genomic DNA of the TAP1 gene. In some embodiments, the modification is a deletion of a contiguous stretch of genomic DNA of the TAP1 gene. In some embodiments, the TAP1 gene is knocked out.

In some embodiments, the provided modified pluripotent stem cells comprise a modification of one or more target polynucleotide sequence that regulate MHC class II molecule expression. Exemplary methods for reducing expression of MHC class II are described in sections below. In some embodiments, the cell comprises a genetic editing modification to the CIITA gene. In some embodiments, the cell comprises a genetic editing modification to the CD74 gene.

In some embodiments, a modification that reduces expression of an MHC class II molecule is a modification that reduces expression of CIITA. In some embodiments, the modification that reduces CIITA expression reduces CIITA mRNA expression. In some embodiments, the reduced mRNA expression of CIITA is relative to an unmodified or wild-type cell of the same cell type that does not comprise the modification. In some embodiments, the mRNA expression of CIITA is reduced by more than about 5%, such as reduced by more than about any of 10%, 20%, 30%, 40%, 50%, 60%, 70%, 80%, 90%, or more. In some embodiments, the mRNA expression of CIITA is reduced by up to about 100%, such as reduced by up to about any of 90%, 80%, 70%, 60%, 50%, 40%, 30%, 20%, 10%, 5%, or less. In some embodiments, the mRNA expression of CIITA is reduced by any of about 5%, 10%, 20%, 30%, 40%, 50%, 60%, 70%, 80%, 90%, or 100%. In some embodiments, the mRNA expression of CIITA is eliminated (e.g., 0% expression of CIITA mRNA). In some embodiments, the modification that reduces CIITA mRNA expression eliminates CIITA gene activity.

In some embodiments, the modification that reduces CIITA expression reduces CIITA protein expression. In some embodiments, the reduced protein expression of CIITA is relative to an unmodified or wild-type cell of the same cell type that does not comprise the modification. In some embodiments, the protein expression of CIITA is reduced by more than about 5%, such as reduced by more than about any of 10%, 20%, 30%, 40%, 50%, 60%, 70%, 80%, 90%, or more. In some embodiments, the protein expression of CIITA is reduced by up to about 100%, such as reduced by up to about any of 90%, 80%, 70%, 60%, 50%, 40%, 30%, 20%, 10%, 5%, or less. In some embodiments, the protein expression of CIITA is reduced by any of about 5%, 10%, 20%, 30%, 40%, 50%, 60%, 70%, 80%, 90%, or 100%. In some embodiments, the protein expression of CIITA is eliminated (e.g., 0% expression of CIITA protein). In some embodiments, the modification that reduces CIITA protein expression eliminates CIITA gene activity.

In some embodiments, the modification that reduces CIITA expression comprises inactivation or disruption of the CIITA gene. In some embodiments, the modification that reduces CIITA expression comprises inactivation or disruption of one allele of the CIITA gene. In some embodiments, the modification that reduces CIITA expression comprises inactivation or disruption comprises inactivation or disruption of both alleles of the CIITA gene.

In some embodiments, the modification comprises inactivation or disruption of one or more CIITA coding sequences in the cell. In some embodiments, the modification comprises inactivation or disruption of all CIITA coding sequences in the cell. In some embodiments, the modification comprises inactivation or disruption comprises an indel in the CIITA gene. In some embodiments, the modification is a frameshift mutation of genomic DNA of the CIITA gene. In some embodiments, the modification is a deletion of genomic DNA of the CIITA gene. In some embodiments, the modification is a deletion of a contiguous stretch of genomic DNA of the CIITA gene. In some embodiments, the CIITA gene is knocked out.

In some embodiments, a modification that reduces expression of an MHC class II molecule is a modification that reduces expression of CD74. In some embodiments, the modification that reduces CD74 expression reduces CD74 mRNA expression. In some embodiments, the reduced mRNA expression of CD74 is relative to an unmodified or wild-type cell of the same cell type that does not comprise the modification. In some embodiments, the mRNA expression of CD74 is reduced by more than about 5%, such as reduced by more than about any of 10%, 20%, 30%, 40%, 50%, 60%, 70%, 80%, 90%, or more. In some embodiments, the mRNA expression of CD74 is reduced by up to about 100%, such as reduced by up to about any of 90%, 80%, 70%, 60%, 50%, 40%, 30%, 20%, 10%, 5%, or less. In some embodiments, the mRNA expression of CD74 is reduced by any of about 5%, 10%, 20%, 30%, 40%, 50%, 60%, 70%, 80%, 90%, or 100%. In some embodiments, the mRNA expression of CD74 is eliminated (e.g., 0% expression of CD74 mRNA). In some embodiments, the modification that reduces CD74 mRNA expression eliminates CD74 gene activity.

In some embodiments, the modification that reduces CD74 expression reduces CD74 protein expression. In some embodiments, the reduced protein expression of CD74 is relative to an unmodified or wild-type cell of the same cell type that does not comprise the modification. In some embodiments, the protein expression of CD74 is reduced by more than about 5%, such as reduced by more than about any of 10%, 20%, 30%, 40%, 50%, 60%, 70%, 80%, 90%, or more. In some embodiments, the protein expression of CD74 is reduced by up to about 100%, such as reduced by up to about any of 90%, 80%, 70%, 60%, 50%, 40%, 30%, 20%, 10%, 5%, or less. In some embodiments, the protein expression of CD74 is reduced by any of about 5%, 10%, 20%, 30%, 40%, 50%, 60%, 70%, 80%, 90%, or 100%. In some embodiments, the protein expression of CD74 is eliminated (e.g., 0% expression of CD74 protein). In some embodiments, the modification that reduces CD74 protein expression eliminates CD74 gene activity.

In some embodiments, the modification that reduces CD74 expression comprises inactivation or disruption of the CD74 gene. In some embodiments, the modification that reduces CD74 expression comprises inactivation or disruption of one allele of the CD74 gene. In some embodiments, the modification that reduces CD74 expression comprises inactivation or disruption comprises inactivation or disruption of both alleles of the CD74 gene.

In some embodiments, the modification comprises inactivation or disruption of one or more CD74 coding sequences in the cell. In some embodiments, the modification comprises inactivation or disruption of all CD74 coding sequences in the cell. In some embodiments, the modification comprises inactivation or disruption comprises an indel in the CD74 gene. In some embodiments, the modification is a frameshift mutation of genomic DNA of the CD74 gene. In some embodiments, the modification is a deletion of genomic DNA of the CD74 gene. In some embodiments, the modification is a deletion of a contiguous stretch of genomic DNA of the CD74 gene. In some embodiments, the CD74 gene is knocked out.

In some embodiments, the provided modified cells comprise a modification of one or more target polynucleotide sequence that regulate expression of MHC class I molecules and MHC class II molecules. Exemplary methods for reducing expression of MHC class I molecules and MHC class II molecules including any as described in sections below. In some embodiments, the cell comprises genetic editing modifications to the B2M and NLRC5 genes. In some embodiments, the cell comprises genetic editing modifications to the CIITA and NLRC5 genes. In some embodiments, the cell comprises genetic editing modifications to the B2M and CIITA genes. In particular embodiments, the cell comprises genetic editing modifications to the B2M, CIITA and NLRC5 genes.

### 2) CD142

In certain aspects, the technology disclosed herein modulate (e.g., reduce or eliminate) the expression of CD142, which is also known as tissue factor, factor III, and F3. In some embodiments, the modulation occurs using a CRISPR/Cas system.

In some embodiments, the target polynucleotide sequence is CD142 or a variant of CD142. In some embodiments, the target polynucleotide sequence is a homolog of CD142. In some embodiments, the target polynucleotide sequence is an ortholog of CD142.

In some embodiments, the cells outlined herein comprise a modification targeting the CD142 gene. In some embodiments, the modification targeting the CD142 gene by the rare-cutting endonuclease comprises a Cas protein or a polynucleotide encoding a Cas protein, and at least one guide ribonucleic acid (gRNA) sequence for specifically targeting the CD142 gene. Useful methods for identifying gRNA sequences to target CD142 are described below.

Assays to test whether the CD142 gene has been inactivated are known and described herein. In one embodiment, the resulting modification of the CD142 gene by PCR and the reduction of CD142 expression can be assays by FACS analysis. In another embodiment, CD142 protein expression is detected using a Western blot of cells lysates probed with antibodies to the CD142 protein. In another embodiment, reverse transcriptase polymerase chain reactions (RT-PCR) are used to confirm the presence of the inactivating modification.

Useful genomic, polynucleotide and polypeptide information about the human CD142 are provided in, for example, the GeneCard Identifier GC01M094530, HGNC No. 3541, NCBI Gene ID 2152, NCBI RefSeq Nos. NM_001178096.1, NM_001993.4, NP_001171567.1, and NP_001984.1, UniProt No. P13726, and the like.

### 3) PD-1

In some embodiments, the target polynucleotide sequence is PD-1 or a variant of PD-1. In some embodiments, the target polynucleotide sequence is a homolog of PD-1. In some embodiments, the target polynucleotide sequence is an ortholog of PD-1.

In some embodiments, the cells outlined herein comprise a genetic modification targeting the gene encoding the programmed cell death protein 1 (PD-1) protein or the PDCD1 gene. In certain embodiments, primary T cells comprise a genetic modification targeting the PDCD1 gene. The genetic modification can reduce expression of PD-1 polynucleotides and PD-1 polypeptides in T cells includes primary T cells and CAR-T cells. In some embodiments, the genetic modification targeting the PDCD1 gene by the rare-cutting endonuclease comprises a Cas protein or a polynucleotide encoding a Cas protein, and at least one guide ribonucleic acid (gRNA) sequence for specifically targeting the PDCD1 gene. Useful methods for identifying gRNA sequences to target PD-1 are described below.

Assays to test whether the PDCD1 gene has been inactivated are known and described herein. In some embodiments, the resulting genetic modification of the PDCD1 gene by PCR and the reduction of PD-1 expression can be assays by FACS analysis. In another embodiment, PD-1 protein expression is detected using a Western blot of cells lysates probed with antibodies to the PD-1 protein. In another embodiment, reverse transcriptase polymerase chain reactions (RT-PCR) are used to confirm the presence of the inactivating genetic modification.

Useful genomic, polynucleotide and polypeptide information about human PD-1 including the PDCD1 gene are provided in, for example, the GeneCard Identifier GC02M241849, HGNC No. 8760, NCBI Gene ID 5133, Uniprot No. Q15116, and NCBI RefSeq Nos. NM_005018.2 and NP_005009.2.

b. Methods of Inactivating or Disrupting Genes (e.g., to Reduce Expression)

In some embodiments, the cells provided herein are modified (e.g., genetically modified) to inactivate or disrupt one or more target polynucleotides or proteins as described. In some embodiments, the cells provided herein are modified (e.g., genetically modified) to reduce expression of the one or more target polynucleotides or proteins as described. In some embodiments, the cell that is modified with the one or more modification to reduce (e.g., eliminate) expression of a polynucleotide or protein is any source cell as described herein. In certain embodiments, the modified pluripotent stem cells (e.g., differentiated cells such as beta islet cells) disclosed herein comprise one or more modifications to reduce expression of one or more target polynucleotides. Non-limiting examples of the one or more target polynucleotides include any as described above, such as CIITA, B2M, CD142, NLRC5, HLA-A, HLA-B, HLA-C, LRC5, RFX-ANK, RFX5, RFX-AP, NFY-A, NFY-B, NFY-C, IRF1, and TAP1. In some embodiment, the target polynucleotide may be CD74. In some embodiments, the modifications to reduce expression of the one or more target polynucleotides is combined with one or more modifications to increase expression of a desired transgene. In some embodiments, the modifications create modified cells that are immune-privileged or hypoimmunogenic cells. By modulating (e.g., reducing or deleting) expression of one or a plurality of the target polynucleotides, such cells exhibit decreased immune activation when engrafted into a recipient subject. In some embodiments, the cell is considered hypoimmunogenic, e.g., in a recipient subject or patient upon administration.

Any method for reducing expression of a target polynucleotide may be used. In some embodiments, the modifications result in permanent elimination or reduction in expression of the target polynucleotide. For instance, in some embodiments, the target polynucleotide or gene is disrupted by introducing a DNA break in the target polynucleotide, such as by using a targeting endonuclease. In other embodiments, the modifications result in transient reduction in expression of the target polynucleotide. For instance, in some embodiments gene repression is achieved using an inhibitory nucleic acid that is complementary to the target polynucleotide to selectively suppress or repress expression of the gene, for instance using antisense techniques, such as by RNA interference (RNAi), short interfering RNA (siRNA), short hairpin (shRNA), and/or ribozymes.

In some embodiments, the target polynucleotide sequence is a genomic sequence. In some embodiments, the target polynucleotide sequence is a human genomic sequence. In some embodiments, the target polynucleotide sequence is a mammalian genomic sequence. In some embodiments, the target polynucleotide sequence is a vertebrate genomic sequence.

In some embodiments, gene disruption is carried out by induction of one or more double-stranded breaks and/or one or more single-stranded breaks in the gene, typically in a targeted manner. In some embodiments, the double-stranded or single-stranded breaks are made by a nuclease, e.g., an endonuclease, such as a gene-targeted nuclease. In some embodiments, the targeted nuclease is selected from zinc finger nucleases (ZFN), transcription activator-like effector nucleases (TALENs), and RNA-guided nucleases such as a CRISPR-associated nuclease (Cas), specifically designed to be targeted to the sequence of a gene or a portion thereof. In some embodiments, the targeted nuclease generates double-stranded or single-stranded breaks that then undergo repair through error prone non-homologous end joining (NHEJ) or, in some cases, precise homology directed repair (HDR) in which a template is used. In some embodiments, the targeted nuclease generates DNA double strand breaks (DSBs). In some embodiments, the process of producing and repairing the breaks is typically error prone and results in insertions and deletions (indels) of DNA bases from NHEJ repair. In some embodiments, the modification may induce a deletion, insertion, or mutation of the nucleotide sequence of the target gene. In some cases, the modification may result in a frameshift mutation, which can result in a premature stop codon. In examples of nuclease-mediated gene editing the targeted edits occur on both alleles of the gene resulting in a biallelic disruption or edit of the gene. In some embodiments, all alleles of the gene are targeted by the gene editing. In some embodiments, modification with a targeted nuclease, such as using a CRISPR/Cas system, leads to complete knockout of the gene. In some embodiments, the nuclease, such as a rare-cutting endonuclease, is introduced into a cell containing the target polynucleotide sequence. The nuclease may be introduced into the cell in the form of a nucleic acid encoding the nuclease. The process of introducing the nucleic acids into cells can be achieved by any suitable technique. Suitable techniques include calcium phosphate or lipid-mediated transfection, electroporation, and transduction or infection using a viral vector. In some embodiments, the nucleic acid that is introduced into the cell is DNA. In some embodiments, the nuclease is introduced into the cell in the form of a protein. For instance, in the case of a CRISPR/Cas system a ribonucleoprotein (RNP) may be introduced into the cell.

In some embodiments, the modification occurs using a CRISPR/Cas system. Any CRISPR/Cas system that is capable of altering a target polynucleotide sequence in a cell can be used. Such CRISPR-Cas systems can employ a variety of Cas proteins (Haft et al. PLoS Comput Biol. 2005; 1(6)e60). The molecular machinery of such Cas proteins that allows the CRISPR/Cas system to alter target polynucleotide sequences in cells include RNA binding proteins, endo- and exo-nucleases, helicases, and polymerases. In some embodiments, the CRISPR/Cas system is a CRISPR type I system. In some embodiments, the CRISPR/Cas system is a CRISPR type II system. In some embodiments, the CRISPR/Cas system is a CRISPR type V system.

The CRISPR/Cas systems include targeted systems that can be used to alter any target polynucleotide sequence in a cell. In some embodiments, a CRISPR/Cas system provided herein includes a Cas protein and one or more, such as at least one to two, ribonucleic acids (e.g., guide RNA (gRNA)) that are capable of directing the Cas protein to and hybridizing to a target motif of a target polynucleotide sequence.

In some embodiments, a Cas protein comprises one or more amino acid substitutions or modifications. In some embodiments, the one or more amino acid substitutions comprises a conservative amino acid substitution. In some instances, substitutions and/or modifications can prevent or reduce proteolytic degradation and/or extend the half-life of the polypeptide in a cell. In some embodiments, the Cas protein can comprise a peptide bond replacement (e.g., urea, thiourea, carbamate, sulfonyl urea, etc.). In some embodiments, the Cas protein can comprise a naturally occurring amino acid. In some embodiments, the Cas protein can comprise an alternative amino acid (e.g., D-amino acids, beta-amino acids, homocysteine, phosphoserine, etc.). In some embodiments, a Cas protein can comprise a modification to include a moiety (e.g., PEGylation, glycosylation, lipidation, acetylation, end-capping, etc.).

In some embodiments, a Cas protein comprises a core Cas protein. Exemplary Cas core proteins include, but are not limited to Cas1, Cas2, Cas3, Cas4, Cas5, Cas6, Cas7, Cas8 and Cas9. In some embodiments, a Cas protein comprises a Cas protein of an E. coli subtype (also known as CASS2). Exemplary Cas proteins of the E. Coli subtype include, but are not limited to Cse1, Cse2, Cse3, Cse4, and Cas5e. In some embodiments, a Cas protein comprises a Cas protein of the Ypest subtype (also known as CASS3). Exemplary Cas proteins of the Ypest subtype include, but are not limited to Csy1, Csy2, Csy3, and Csy4. In some embodiments, a Cas protein comprises a Cas protein of the Nmeni subtype (also known as CASS4). Exemplary Cas proteins of the Nmeni subtype include, but are not limited to Csn1 and Csn2. In some embodiments, a Cas protein comprises a Cas protein of the Dvulg subtype (also known as CASS1). Exemplary Cas proteins of the Dvulg subtype include Csd1, Csd2, and Cas5d. In some embodiments, a Cas protein comprises a Cas protein of the Tneap subtype (also known as CASS7). Exemplary Cas proteins of the Tneap subtype include, but are not limited to, Cst1, Cst2, Cas5t. In some embodiments, a Cas protein comprises a Cas protein of the Hmari subtype. Exemplary Cas proteins of the Hmari subtype include, but are not limited to Csh1, Csh2, and Cas5h. In some embodiments, a Cas protein comprises a Cas protein of the Apern subtype (also known as CASS5). Exemplary Cas proteins of the Apern subtype include, but are not limited to Csa1, Csa2, Csa3, Csa4, Csa5, and Cas5a. In some embodiments, a Cas protein comprises a Cas protein of the Mtube subtype (also known as CASS6). Exemplary Cas proteins of the Mtube subtype include, but are not limited to Csm1, Csm2, Csm3, Csm4, and Csm5. In some embodiments, a Cas protein comprises a RAMP module Cas protein. Exemplary RAMP module Cas proteins include, but are not limited to, Cmr1, Cmr2, Cmr3, Cmr4, Cmr5, and Cmr6. See, e.g., Klompe et al., Nature 571, 219-225 (2019); Strecker et al., Science 365, 48-53 (2019).

In some embodiments, CRISPR systems of the present disclosure comprise TnpB polypeptides. In some embodiments, TnpB polypeptides may comprise a Ruv-C-like domain. The RuvC domain may be a split RuvC domain comprising RuvC-I, RuvC-II, and RuvC-III subdomains. In some embodiments, a TnpB may further comprise one or more of a HTH domain, a bridge helix domain, and a zinc finger domain. TnpB polypeptides do not comprise an HNH domain. In some embodiments, a TnpB protein comprises, starting at the N-terminus: a HTH domain, a RuvC-I subdomain, a bridge helix domain, a RuvC-II sub-domain, a zinger finger domain, and a RuvC-III sub-domain. In some embodiments, a RuvC-III sub-domain forms the C-terminus of a TnpB polypeptide. In some embodiments, a TnpB polypeptide is from Epsilonproteobacteria bacterium, Actinoplanes lobatus strain DSM 43150, Actinomadura celluolosilytica strain DSM 45823, Actinomadura namibiensis strain DSM 44197, Alicyclobacillus macrosprangiidus strain DSM 17980, Lipingzhangella halophila strain DSM 102030, or Ktedonobacter recemifer. In some embodiments, a TnpB polypeptide is from Ktedonobacter racemifer, or comprises a conserved RNA region with similarity to the 5′ ITR of K. racemifer TnpB loci. In some embodiments, a TnpB may comprise a Fanzor protein, a TnpB homolog found in eukaryotic genomes. In some embodiments, a CRISPR system comprising a TnpB polypeptide binds a target adjacent motif (TAM) sequence 5′ of a target polynucleotide. In some embodiments, a TAM is a transposon-associated motif. In some embodiments, a TAM sequence comprises TCA. In some embodiments, a TAM sequence comprises TTCAN. In some embodiments, a TAM sequence comprises TTGAT. In some embodiments, a TAM sequence comprises ATAAA.

In some embodiments, the methods for genetically modifying cells to knock out, knock down, or otherwise modify one or more genes comprise using a site-directed nuclease, including, for example, zinc finger nucleases (ZFNs), transcription activator-like effector nucleases (TALENs), meganucleases, transposases, and clustered regularly interspaced short palindromic repeat (CRISPR)/Cas systems

ZFNs are fusion proteins comprising an array of site-specific DNA binding domains adapted from zinc finger-containing transcription factors attached to the endonuclease domain of the bacterial FokI restriction enzyme. A ZFN may have one or more (e.g., 1, 2, 3, 4, 5, 6, 7, 8, 9, 10, or more) of the DNA binding domains or zinc finger domains. See, e.g., Carroll et al., Genetics Society of America (2011) 188:773-782; Kim et al., Proc. Natl. Acad. Sci. USA (1996) 93:1156-1160. Each zinc finger domain is a small protein structural motif stabilized by one or more zinc ions and usually recognizes a 3- to 4-bp DNA sequence. Tandem domains can thus potentially bind to an extended nucleotide sequence that is unique within a cell's genome.

Various zinc fingers of known specificity can be combined to produce multi-finger polypeptides which recognize about 6, 9, 12, 15, or 18-bp sequences. Various selection and modular assembly techniques are available to generate zinc fingers (and combinations thereof) recognizing specific sequences, including phage display, yeast one-hybrid systems, bacterial one-hybrid and two-hybrid systems, and mammalian cells. Zinc fingers can be engineered to bind a predetermined nucleic acid sequence. Criteria to engineer a zinc finger to bind to a predetermined nucleic acid sequence are known in the art. See, e.g., Sera et al., Biochemistry (2002) 41:7074-7081; Liu et al., Bioinformatics (2008) 24:1850-1857.

ZFNs containing FokI nuclease domains or other dimeric nuclease domains function as a dimer. Thus, a pair of ZFNs are required to target non-palindromic DNA sites. The two individual ZFNs must bind opposite strands of the DNA with their nucleases properly spaced apart. See Bitinaite et al., Proc. Natl. Acad. Sci. USA (1998) 95:10570-10575. To cleave a specific site in the genome, a pair of ZFNs are designed to recognize two sequences flanking the site, one on the forward strand and the other on the reverse strand. Upon binding of the ZFNs on either side of the site, the nuclease domains dimerize and cleave the DNA at the site, generating a DSB with 5′ overhangs. HDR can then be utilized to introduce a specific mutation, with the help of a repair template containing the desired mutation flanked by homology arms. The repair template is usually an exogenous double-stranded DNA vector introduced to the cell. See Miller et al., Nat. Biotechnol. (2011) 29:143-148; Hockemeyer et al., Nat. Biotechnol. (2011) 29:731-734.

TALENs are another example of an artificial nuclease which can be used to edit a target gene. TALENs are derived from DNA binding domains termed TALE repeats, which usually comprise tandem arrays with 10 to 30 repeats that bind and recognize extended DNA sequences. Each repeat is 33 to 35 amino acids in length, with two adjacent amino acids (termed the repeat-variable di-residue, or RVD) conferring specificity for one of the four DNA base pairs. Thus, there is a one-to-one correspondence between the repeats and the base pairs in the target DNA sequences.

TALENs are produced artificially by fusing one or more TALE DNA binding domains (e.g., 1, 2, 3, 4, 5, 6, 7, 8, 9, 10 or more) to a nuclease domain, for example, a FokI endonuclease domain. See Zhang, Nature Biotech. (2011) 29:149-153. Several mutations to FokI have been made for its use in TALENs; these, for example, improve cleavage specificity or activity. See Cermak et al., Nucl. Acids Res. (2011) 39: e82; Miller et al., Nature Biotech. (2011) 29:143-148; Hockemeyer et al., Nature Biotech. (2011) 29:731-734; Wood et al., Science (2011) 333:307; Doyon et al., Nature Methods (2010) 8:74-79; Szczepek et al., Nature Biotech (2007) 25:786-793; Guo et al., J. Mol. Biol. (2010) 200:96. The FokI domain functions as a dimer, requiring two constructs with unique DNA binding domains for sites in the target genome with proper orientation and spacing. Both the number of amino acid residues between the TALE DNA binding domain and the FokI nuclease domain and the number of bases between the two individual TALEN binding sites appear to be important parameters for achieving high levels of activity. Miller et al., Nature Biotech. (2011) 29:143-148.

By combining engineered TALE repeats with a nuclease domain, a site-specific nuclease can be produced specific to any desired DNA sequence. Similar to ZFNs, TALENs can be introduced into a cell to generate DSBs at a desired target site in the genome, and so can be used to knock out genes or knock in mutations in similar, HDR-mediated pathways. See Boch, Nature Biotech. (2011) 29:135-136; Boch et al., Science (2009) 326:1509-1512; Moscou et al., Science (2009) 326:3501.

Meganucleases are enzymes in the endonuclease family which are characterized by their capacity to recognize and cut large DNA sequences (from 14 to 40 base pairs). Meganucleases are grouped into families based on their structural motifs which affect nuclease activity and/or DNA recognition. The most widespread and best known meganucleases are the proteins in the LAGLIDADG family, which owe their name to a conserved amino acid sequence. See Chevalier et al., Nucleic Acids Res. (2001) 29 (18): 3757-3774. On the other hand, the GIY-YIG family members have a GIY-YIG module, which is 70-100 residues long and includes four or five conserved sequence motifs with four invariant residues, two of which are required for activity. See Van Roey et al., Nature Struct. Biol. (2002) 9:806-811. The His-Cys family meganucleases are characterized by a highly conserved series of histidines and cysteines over a region encompassing several hundred amino acid residues. See Chevalier et al., Nucleic Acids Res. (2001) 29 (18): 3757-3774. Members of the NHN family are defined by motifs containing two pairs of conserved histidines surrounded by asparagine residues. See Chevalier et al., Nucleic Acids Res. (2001) 29 (18): 3757-3774.

Because the chance of identifying a natural meganuclease for a particular target DNA sequence is low due to the high specificity requirement, various methods including mutagenesis and high throughput screening methods have been used to create meganuclease variants that recognize unique sequences. Strategies for engineering a meganuclease with altered DNA-binding specificity, e.g., to bind to a predetermined nucleic acid sequence are known in the art. See, e.g., Chevalier et al., Mol. Cell. (2002) 10:895-905; Epinat et al., Nucleic Acids Res (2003) 31:2952-2962; Silva et al., J Mol. Biol. (2006) 361:744-754; Seligman et al., Nucleic Acids Res (2002) 30:3870-3879; Sussman et al., J Mol Biol (2004) 342:31-41; Doyon et al., J Am Chem Soc (2006) 128:2477-2484; Chen et al., Protein Eng Des Sel (2009) 22:249-256; Arnould et al., J Mol Biol. (2006) 355:443-458; Smith et al., Nucleic Acids Res. (2006) 363 (2): 283-294.

Like ZFNs and TALENs, Meganucleases can create DSBs in the genomic DNA, which can create a frame-shift mutation if improperly repaired, e.g., via NHEJ, leading to a decrease in the expression of a target gene in a cell. Alternatively, foreign DNA can be introduced into the cell along with the meganuclease. Depending on the sequences of the foreign DNA and chromosomal sequence, this process can be used to modify the target gene. See Silva et al., Current Gene Therapy (2011) 11:11-27.

Transposases are enzymes that bind to the end of a transposon and catalyze its movement to another part of the genome by a cut and paste mechanism or a replicative transposition mechanism. By linking transposases to other systems such as the CRISPR/Cas system, new gene editing tools can be developed to enable site specific insertions or manipulations of the genomic DNA. There are two known DNA integration methods using transposons which use a catalytically inactive Cas effector protein and Tn7-like transposons. The transposase-dependent DNA integration does not provoke DSBs in the genome, which may guarantee safer and more specific DNA integration.

The CRISPR system was originally discovered in prokaryotic organisms (e.g., bacteria and archaea) as a system involved in defense against invading phages and plasmids that provides a form of acquired immunity. Now it has been adapted and used as a popular gene editing tool in research and clinical applications.

CRISPR/Cas systems generally comprise at least two components: one or more guide RNAs (gRNAs) and a Cas protein. The Cas protein is a nuclease that introduces a DSB into the target site. CRISPR-Cas systems fall into two major classes: class 1 systems use a complex of multiple Cas proteins to degrade nucleic acids; class 2 systems use a single large Cas protein for the same purpose. Class 1 is divided into types I, III, and IV; class 2 is divided into types II, V, and VI. Different Cas proteins adapted for gene editing applications include, but are not limited to, Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, and Mad7. The most widely used Cas9 is a type II Cas protein and is described herein as illustrative. These Cas proteins may be originated from different source species. For example, Cas9 can be derived from S. pyogenes or S. aureus.

In the original microbial genome, the type II CRISPR system incorporates sequences from invading DNA between CRISPR repeat sequences encoded as arrays within the host genome. Transcripts from the CRISPR repeat arrays are processed into CRISPR RNAs (crRNAs) each harboring a variable sequence transcribed from the invading DNA, known as the “protospacer” sequence, as well as part of the CRISPR repeat. Each crRNA hybridizes with a second transactivating CRISPR RNA (tracrRNA), and these two RNAs form a complex with the Cas9 nuclease. The protospacer-encoded portion of the crRNA directs the Cas9 complex to cleave complementary target DNA sequences, provided that they are adjacent to short sequences known as “protospacer adjacent motifs” (PAMs).

Since its discovery, the CRISPR system has been adapted for inducing sequence specific DSBs and targeted genome editing in a wide range of cells and organisms spanning from bacteria to eukaryotic cells including human cells. In its use in gene editing applications, artificially designed, synthetic gRNAs have replaced the original crRNA: tracrRNA complex. For example, the gRNAs can be single guide RNAs (sgRNAs) composed of a crRNA, a tetraloop, and a tracrRNA. The crRNA usually comprises a complementary region (also called a spacer, usually about 20 nucleotides in length) that is user-designed to recognize a target DNA of interest. The tracrRNA sequence comprises a scaffold region for Cas nuclease binding. The crRNA sequence and the tracrRNA sequence are linked by the tetraloop and each have a short repeat sequence for hybridization with each other, thus generating a chimeric sgRNA. One can change the genomic target of the Cas nuclease by simply changing the spacer or complementary region sequence present in the gRNA. The complementary region will direct the Cas nuclease to the target DNA site through standard RNA-DNA complementary base pairing rules.

In order for the Cas nuclease to function, there must be a PAM immediately downstream of the target sequence in the genomic DNA. Recognition of the PAM by the Cas protein is thought to destabilize the adjacent genomic sequence, allowing interrogation of the sequence by the gRNA and resulting in gRNA-DNA pairing when a matching sequence is present. The specific sequence of PAM varies depending on the species of the Cas gene. For example, the most commonly used Cas9 nuclease derived from S. pyogenes recognizes a PAM sequence of 5′-NGG-3′ or, at less efficient rates, 5′-NAG-3′, where “N” can be any nucleotide. Other Cas nuclease variants with alternative PAMs have also been characterized and successfully used for genome editing, which are summarized in Table 1a below.

TABLE 1a
Exemplary Cas nuclease
variants and their PAM sequences

| CRISPR | | PAM Sequence |
|---|---|---|
| Nuclease | Source Organism | (5′→3′) |
| SpCas9 | Streptococcus | NGG or NAG |
| | pyogenes | |
| SaCas9 | Staphylococcus | NGRRT or |
| | aureus | NGRRN |
| NmeCas9 | Neisseria | NNNNGATT |
| | meningitidis | |
| CjCas9 | Campylobacter jejuni | NNNNRYAC |
| StCas9 | Streptococcus | NNAGAAW |
| | thermophilus | |
| TdCas9 | Treponema denticola | NAAAAC |
| LbCas12a (Cpf1) | Lachnospiraceae | TTTV |
| | bacterium | |
| AsCas12a (Cpf1) | Acidaminococcus sp. | TTTV |
| AacCas12b | Alicyclobacillus | TTN |
| | acidiphilus | |
| BhCas12b v4 | Bacillus hisashii | ATTN, TTTN, |
| | | or GTTN |

R = A or G;
Y = C or T;
W = A or T;
V = A or C or G;
N = any base

In some embodiments, Cas nucleases may comprise one or more mutations to alter their activity, specificity, recognition, and/or other characteristics. For example, the Cas nuclease may have one or more mutations that alter its fidelity to mitigate off-target effects (e.g., eSpCas9, SpCas9-HF1, HypaSpCas9, HeFSpCas9, and evoSpCas9 high-fidelity variants of SpCas9). For another example the Cas nuclease may have one or more mutations that alter its PAM specificity.

In some embodiments, a Cas protein comprises any one of the Cas proteins described herein or a functional portion thereof. As used herein, “functional portion” refers to a portion of a peptide which retains its ability to complex with at least one ribonucleic acid (e.g., guide RNA (gRNA)) and cleave a target polynucleotide sequence. In some embodiments, the functional portion comprises a combination of operably linked Cas9 protein functional domains selected from the group consisting of a DNA binding domain, at least one RNA binding domain, a helicase domain, and an endonuclease domain. In some embodiments, the functional portion comprises a combination of operably linked Cas12a (also known as Cpf1) protein functional domains selected from the group consisting of a DNA binding domain, at least one RNA binding domain, a helicase domain, and an endonuclease domain. In some embodiments, the functional domains form a complex. In some embodiments, a functional portion of the Cas9 protein comprises a functional portion of a RuvC-like domain. In some embodiments, a functional portion of the Cas9 protein comprises a functional portion of the HNH nuclease domain. In some embodiments, a functional portion of the Cas 12a protein comprises a functional portion of a RuvC-like domain.

In some embodiments, suitable Cas proteins include, but are not limited to, Cas0, Cas12a (i.e., Cpf1), Cas12b, Cas12i, CasX, and Mad7.

In some embodiments, exogenous Cas protein can be introduced into the cell in polypeptide form. In certain embodiments, Cas proteins can be conjugated to or fused to a cell-penetrating polypeptide or cell-penetrating peptide. As used herein, “cell-penetrating polypeptide” and “cell-penetrating peptide” refers to a polypeptide or peptide, respectively, which facilitates the uptake of molecule into a cell. The cell-penetrating polypeptides can contain a detectable label.

In certain embodiments, Cas proteins can be conjugated to or fused to a charged protein (e.g., that carries a positive, negative or overall neutral electric charge). Such linkage may be covalent. In some embodiments, the Cas protein can be fused to a superpositively charged GFP to significantly increase the ability of the Cas protein to penetrate a cell (Cronican et al. ACS Chem Biol. 2010; 5 (8): 747-52). In certain embodiments, the Cas protein can be fused to a protein transduction domain (PTD) to facilitate its entry into a cell. Exemplary PTDs include Tat, oligoarginine, and penetratin. In some embodiments, the Cas9 protein comprises a Cas9 polypeptide fused to a cell-penetrating peptide. In some embodiments, the Cas9 protein comprises a Cas9 polypeptide fused to a PTD. In some embodiments, the Cas9 protein comprises a Cas9 polypeptide fused to a tat domain. In some embodiments, the Cas9 protein comprises a Cas9 polypeptide fused to an oligoarginine domain. In some embodiments, the Cas9 protein comprises a Cas9 polypeptide fused to a penetrating domain. In some embodiments, the Cas9 protein comprises a Cas9 polypeptide fused to a superpositively charged GFP. In some embodiments, the Cas 12a protein comprises a Cas12a polypeptide fused to a cell-penetrating peptide. In some embodiments, the Cas12a protein comprises a Cas12a polypeptide fused to a PTD. In some embodiments, the Cas12a protein comprises a Cas 12a polypeptide fused to a tat domain. In some embodiments, the Cas12a protein comprises a Cas12a polypeptide fused to an oligoarginine domain. In some embodiments, the Cas12a protein comprises a Cas12a polypeptide fused to a penetrating domain. In some embodiments, the Cas12a protein comprises a Cas12a polypeptide fused to a superpositively charged GFP.

In some embodiments, the Cas protein can be introduced into a cell containing the target polynucleotide sequence in the form of a nucleic acid encoding the Cas protein. The process of introducing the nucleic acids into cells can be achieved by any suitable technique. Suitable techniques include calcium phosphate or lipid-mediated transfection, electroporation, and transduction or infection using a viral vector. In some embodiments, the nucleic acid comprises DNA. In some embodiments, the nucleic acid comprises a modified DNA, as described herein. In some embodiments, the nucleic acid comprises mRNA. In some embodiments, the nucleic acid comprises a modified mRNA, as described herein (e.g., a synthetic, modified mRNA).

In provided embodiments, a CRISPR/Cas system generally includes two components: one or more guide RNA (gRNA) and a Cas protein. In some embodiments, the Cas protein is complexed with the one or more, such as one to two, ribonucleic acids (e.g., guide RNA (gRNA)). In some embodiments, the Cas protein is complexed with two ribonucleic acids. In some embodiments, the Cas protein is complexed with one ribonucleic acid. In some embodiments, the Cas protein is encoded by a modified nucleic acid, as described herein (e.g., a synthetic, modified mRNA).

In some embodiments, gRNAs are short synthetic RNAs composed of a scaffold sequence for Cas binding and a user-designed spacer or complementary portion designated crRNA. The cRNA is composed of a crRNA targeting sequence (herein after also called a gRNA targeting sequence; usually about 20 nucleotides in length) that defines the genomic target to be modified and a region of crRNA repeat (e.g. GUUUUAGAGCUA; SEQ ID NO:19). One can change the genomic target of the Cas protein by simply changing the complementary portion sequence (e.g., gRNA targeting sequence) present in the gRNA. In some embodiments the scaffold sequence for Cas binding is made up of a tracrRNA sequence (e.g. UAGCAAGUUAAAAUAAGGCUAGUCCGUUAUCAACUUGAAAAAGUGGCACCGAGUCGGUG CUUU; SEQ ID NO: 20) that hybridizes to the crRNA through its anti-repeat sequence. The complex between crRNA: tracrRNA recruits the Cas nuclease (e.g., Cas9) and cleaves upstream of a protospacer-adjacent motif (PAM). For the Cas protein to function, there must be a PAM immediately downstream of the target sequence in the genomic DNA. Recognition of the PAM by the Cas protein is thought to destabilize the adjacent genomic sequence, allowing interrogation of the sequence by the gRNA and resulting in gRNA-DNA pairing when a matching sequence is present. The specific sequence of PAM varies depending on the species of the Cas gene. For example, the most commonly used Cas9 nuclease, derived from S. pyogenes, recognizes a PAM sequence of NGG. Other Cas9 variants and other nucleases with alternative PAMs have also been characterized and successfully used for genome editing. Thus, the CRISPR/Cas system can be used to create targeted DSBs at specified genomic loci that are complementary to the gRNA designed for the target loci. The crRNA and tracrRNA can be linked together with a loop sequence (e.g., a tetraloop; GAAA, SEQ ID NO:21) for generation of a gRNA that is a chimeric single guide RNA (sgRNA; Hsu et al. 2013). sgRNA can be generated for DNA-based expression or by chemical synthesis.

In some embodiments, the complementary portion sequences (e.g., gRNA targeting sequence) of the gRNA will vary depending on the target site of interest. In some embodiments, the gRNAs comprise complementary portions specific to a sequence of a gene set forth in Table 1b or Table 1c. In some embodiments, the genomic locus targeted by the gRNAs is located within 4000 bp, within 3500 bp, within 3000 bp, within 2500 bp, within 2000 bp, within 1500 bp, within 1000 bp, or within 500 bp of any of the loci as described.

The methods disclosed herein contemplate the use of any ribonucleic acid that is capable of directing a Cas protein to and hybridizing to a target motif of a target polynucleotide sequence. In some embodiments, at least one of the ribonucleic acids comprises

In some embodiments, the Cas protein is complexed with one to two ribonucleic acids (e.g., guide RNA (gRNA)). In some embodiments, the Cas protein is complexed with two ribonucleic acids. In some embodiments, the Cas protein is complexed with one ribonucleic acid. In some embodiments, the Cas protein is encoded by a modified nucleic acid, as described herein (e.g., a synthetic, modified mRNA).

The methods disclosed herein contemplate the use of any ribonucleic acid that is capable of directing a Cas protein to and hybridizing to a target motif of a target polynucleotide sequence. In some embodiments, at least one of the ribonucleic acids comprises tracrRNA. In some embodiments, at least one of the ribonucleic acids comprises CRISPR RNA (crRNA). In some embodiments, a single ribonucleic acid comprises a guide RNA that directs the Cas protein to and hybridizes to a target motif of the target polynucleotide sequence in a cell. In some embodiments, at least one of the ribonucleic acids comprises a guide RNA that directs the Cas protein to and hybridizes to a target motif of the target polynucleotide sequence in a cell. In some embodiments, both of the one to two ribonucleic acids comprise a guide RNA that directs the Cas protein to and hybridizes to a target motif of the target polynucleotide sequence in a cell. The ribonucleic acids provided herein can be selected to hybridize to a variety of different target motifs, depending on the particular CRISPR/Cas system employed, and the sequence of the target polynucleotide, as will be appreciated by those skilled in the art. The one to two ribonucleic acids can also be selected to minimize hybridization with nucleic acid sequences other than the target polynucleotide sequence. In some embodiments, the one to two ribonucleic acids hybridize to a target motif that contains at least two mismatches when compared with all other genomic nucleotide sequences in the cell. In some embodiments, the one to two ribonucleic acids hybridize to a target motif that contains at least one mismatch when compared with all other genomic nucleotide sequences in the cell. In some embodiments, the one to two ribonucleic acids are designed to hybridize to a target motif immediately adjacent to a deoxyribonucleic acid motif recognized by the Cas protein. In some embodiments, each of the one to two ribonucleic acids are designed to hybridize to target motifs immediately adjacent to deoxyribonucleic acid motifs recognized by the Cas protein which flank a mutant allele located between the target motifs.

In some embodiments, each of the one to two ribonucleic acids comprises guide RNAs that directs the Cas protein to and hybridizes to a target motif of the target polynucleotide sequence in a cell.

In some embodiments, one or two ribonucleic acids (e.g., guide RNAs) are complementary to and/or hybridize to sequences on the same strand of a target polynucleotide sequence. In some embodiments, one or two ribonucleic acids (e.g., guide RNAs) are complementary to and/or hybridize to sequences on the opposite strands of a target polynucleotide sequence. In some embodiments, the one or two ribonucleic acids (e.g., guide RNAs) are not complementary to and/or do not hybridize to sequences on the opposite strands of a target polynucleotide sequence. In some embodiments, the one or two ribonucleic acids (e.g., guide RNAs) are complementary to and/or hybridize to overlapping target motifs of a target polynucleotide sequence. In some embodiments, the one or two ribonucleic acids (e.g., guide RNAs) are complementary to and/or hybridize to offset target motifs of a target polynucleotide sequence.

In some embodiments, nucleic acids encoding Cas protein and nucleic acids encoding the at least one to two ribonucleic acids are introduced into a cell via viral transduction (e.g., lentiviral transduction). In some embodiments, the Cas protein is complexed with 1-2 ribonucleic acids. In some embodiments, the Cas protein is complexed with two ribonucleic acids. In some embodiments, the Cas protein is complexed with one ribonucleic acid. In some embodiments, the Cas protein is encoded by a modified nucleic acid, as described herein (e.g., a synthetic, modified mRNA).

Exemplary gRNA targeting sequences useful for CRISPR/Cas-based targeting of genes described herein are provided in Table 1b or Table 1c.

The sequences can be found in WO2016183041 filed May 9, 2016, the disclosure including the Tables, Appendices, and Sequence Listing is incorporated herein by reference in its entirety.

TABLE 1b
Exemplary gRNA targeting sequences useful for targeting genes

| Gene | | |
|---|---|---|
| Name | SEQ ID NO: | WO2016183041 |
| HLA-A | SEQ ID NOs: 2-1418 | Table 8, Appendix 1 |
| HLA-B | SEQ ID NOs: 1419-3277 | Table 9, Appendix 2 |
| HLA-C | SEQ ID NOS: 3278-5183 | Table 10, Appendix 3 |
| RFX-ANK | SEQ ID NOs: 95636-102318 | Table 11, Appendix 4 |
| NFY-A | SEQ ID NOs: 102319-121796 | Table 13, Appendix 6 |
| RFX5 | SEQ ID NOs: 85645-90115 | Table 16, Appendix 9 |
| RFX-AP | SEQ ID NOs: 90116-95635 | Table 17, Appendix 10 |
| NFY-B | SEQ ID NOs: 121797-135112 | Table 20, Appendix 13 |
| NFY-C | SEQ ID NOs: 135113-176601 | Table 22, Appendix 15 |
| IRF1 | SEQ ID NOs: 176602-182813 | Table 23, Appendix 16 |
| TAP1 | SEQ ID NOs: 182814-188371 | Table 24, Appendix 17 |
| CIITA | SEQ ID NOS: 5184-36352 | Table 12, Appendix 5 |
| B2M | SEQ ID NOS: 81240-85644 | Table 15, Appendix 8 |
| NLRC5 | SEQ ID NOS: 36353-81239 | Table 14, Appendix 7 |

Additional exemplary Cas9 guide RNA sequences useful for CRISPR/Cas-based targeting of genes described herein are provided in Table 1C. It will be understood by one of ordinary skill in the art that uracil and thymine can both be represented by ‘t’, instead of ‘u’ for uracil and ‘t’ for thymine; in the context of a ribonucleic acid, it will be understood that ‘t’ is used to represent uracil unless otherwise indicated.

TABLE 1C
Additional exemplary Cas9 guide
RNA sequences useful for targeting genes

| | | | | | SEQ ID |
|---|---|---|---|---|---|
| | | | | | NO of |
| | guide | | Target | gRNA cut | guide |
| Gene | sequence | PAM | site | location | sequence |
| ABO | UCUCUCCAUG | AGG | Exon 7 | chr9: | 29 |
| | UGCAGUAGGA | | | 133,257,541 | |
| FUT1 | CUGGAUGUCG | CGG | Exon 4 | chr19: | 30 |
| | GAGGAGUACG | | | 48,750,822 | |
| RHD | GUCUCCGGAA | AGG | Exon 2 | chr1: | 31 |
| | ACUCGAGGUG | | | 25,284,622 | |
| F3 | ACAGUGUAGA | GGG | Exon 2 | chr1: | 32 |
| (CD142) | CUUGAUUGAC | | | 94,540,281 | |
| B2M | CGUGAGUAAA | TGG | Exon 2 | chr15: | 33 |
| | CCUGAAUCUU | | | 44,715,434 | |
| CIITA | GAUAUUGGCA | TGG | Exon 3 | chr16: | 34 |
| | UAAGCCUCCC | | | 10,895,747 | |
| TRAC | AGAGUCUCUC | CGG | Exon 1 | chr14: | 28 |
| | AGCUGGUACA | | | 22,5547,533 | |

In some embodiments, it is within the level of a skilled artisan to identify new loci and/or gRNA targeting sequences for use in methods of genetic disruption to reduce or eliminate expression of a gene as described. For example, for CRISPR/Cas systems, when an existing gRNA targeting sequence for a particular locus (e.g., within a target gene, e.g. set forth in Table 1b or 1c) is known, an “inch worming” approach can be used to identify additional loci for targeted insertion of transgenes by scanning the flanking regions on either side of the locus for PAM sequences, which usually occurs about every 100 base pairs (bp) across the genome. The PAM sequence will depend on the particular Cas nuclease used because different nucleases usually have different corresponding PAM sequences. The flanking regions on either side of the locus can be between about 500 to 4000 bp long, for example, about 500 bp, about 1000 bp, about 1500 bp, about 2000 bp, about 2500 bp, about 3000 bp, about 3500 bp, or about 4000 bp long. When a PAM sequence is identified within the search range, a new guide can be designed according to the sequence of that locus for use in genetic disruption methods. Although the CRISPR/Cas system is described as illustrative, any gene-editing approaches as described can be used in this method of identifying new loci, including those using ZFNs, TALENS, meganucleases and transposases.

In some embodiments, the cells described herein are made using Transcription Activator-Like Effector Nucleases (TALEN) methodologies. By a “TALE-nuclease” (TALEN) is intended a fusion protein consisting of a nucleic acid-binding domain typically derived from a Transcription Activator Like Effector (TALE) and one nuclease catalytic domain to cleave a nucleic acid target sequence. The catalytic domain is preferably a nuclease domain and more preferably a domain having endonuclease activity, like for instance I-TevI, ColE7, NucA and Fok-I. In a particular embodiment, the TALE domain can be fused to a meganuclease like for instance I-CreI and I-OnuI or functional variant thereof. In a more preferred embodiment, said nuclease is a monomeric TALE-Nuclease. A monomeric TALE-Nuclease is a TALE-Nuclease that does not require dimerization for specific recognition and cleavage, such as the fusions of engineered TAL repeats with the catalytic domain of I-TevI described in WO2012138927. Transcription Activator like Effector (TALE) are proteins from the bacterial species Xanthomonas comprise a plurality of repeated sequences, each repeat comprising di-residues in position 12 and 13 (RVD) that are specific to each nucleotide base of the nucleic acid targeted sequence. Binding domains with similar modular base-per-base nucleic acid binding properties (MBBBD) can also be derived from new modular proteins recently discovered by the applicant in a different bacterial species. The new modular proteins have the advantage of displaying more sequence variability than TAL repeats. Preferably, RVDs associated with recognition of the different nucleotides are HD for recognizing C, NG for recognizing T, NI for recognizing A, NN for recognizing G or A, NS for recognizing A, C, G or T, HG for recognizing T, IG for recognizing T, NK for recognizing G, HA for recognizing C, ND for recognizing C, HI for recognizing C, HN for recognizing G, NA for recognizing G, SN for recognizing G or A and YG for recognizing T, TL for recognizing A, VT for recognizing A or G and SW for recognizing A. In another embodiment, critical amino acids 12 and 13 can be mutated towards other amino acid residues in order to modulate their specificity towards nucleotides A, T, C and G and in particular to enhance this specificity. TALEN kits are sold commercially.

In some embodiments, the cells are manipulated using zinc finger nuclease (ZFN). A “zinc finger binding protein” is a protein or polypeptide that binds DNA, RNA and/or protein, preferably in a sequence-specific manner, as a result of stabilization of protein structure through coordination of a zinc ion. The term zinc finger binding protein is often abbreviated as zinc finger protein or ZFP. The individual DNA binding domains are typically referred to as “fingers.” A ZFP has least one finger, typically two fingers, three fingers, or six fingers. Each finger binds from two to four base pairs of DNA, typically three or four base pairs of DNA. A ZFP binds to a nucleic acid sequence called a target site or target segment. Each finger typically comprises an approximately 30 amino acid, zinc-chelating, DNA-binding subdomain. Studies have demonstrated that a single zinc finger of this class consists of an alpha helix containing the two invariant histidine residues coordinated with zinc along with the two cysteine residues of a single beta turn (see, e.g., Berg & Shi, Science 271:1081-1085 (1996)).

In some embodiments, the cells described herein are made using a homing endonuclease. Such homing endonucleases are well-known to the art (Stoddard 2005). Homing endonucleases recognize a DNA target sequence and generate a single- or double-strand break. Homing endonucleases are highly specific, recognizing DNA target sites ranging from 12 to 45 base pairs (bp) in length, usually ranging from 14 to 40 bp in length. The homing endonuclease may for example correspond to a LAGLIDADG endonuclease, to an HNH endonuclease, or to a GIY-YIG endonuclease. In some embodiments, the homing endonuclease can be an I-CreI variant.

In some embodiments, the cells described herein are made using a meganuclease. Meganucleases are by definition sequence-specific endonucleases recognizing large sequences (Chevalier, B. S. and B. L. Stoddard, Nucleic Acids Res., 2001, 29, 3757-3774). They can cleave unique sites in living cells, thereby enhancing gene targeting by 1000-fold or more in the vicinity of the cleavage site (Puchta et al., Nucleic Acids Res., 1993, 21, 5034-5040; Rouet et al., Mol. Cell. Biol., 1994, 14, 8096-8106; Choulika et al., Mol. Cell. Biol., 1995, 15, 1968-1973; Puchta et al., Proc. Natl. Acad. Sci. USA, 1996, 93, 5055-5060; Sargent et al., Mol. Cell. Biol., 1997, 17, 267-77; Donoho et al., Mol. Cell. Biol, 1998, 18, 4070-4078; Elliott et al., Mol. Cell. Biol., 1998, 18, 93-101; Cohen-Tannoudji et al., Mol. Cell. Biol., 1998, 18, 1444-1448).

In some embodiments, the cells provided herein are made using RNA silencing or RNA interference (RNAi) to knockdown (e.g., decrease, eliminate, or inhibit) the expression of a polypeptide. Useful RNAi methods include those that utilize synthetic RNAi molecules, short interfering RNAs (siRNAs), PIWI-interacting NRAs (piRNAs), short hairpin RNAs (shRNAs), microRNAs (miRNAs), and other transient knockdown methods recognized by those skilled in the art. Reagents for RNAi including sequence specific shRNAs, siRNA, miRNAs and the like are commercially available. For instance, a target polynucleotide, such as any described above, e.g., CIITA, B2M, or NLRC5, can be knocked down in a cell by RNA interference by introducing an inhibitory nucleic acid complementary to a target motif of the target polynucleotide, such as an siRNA, into the cells. In some embodiments, a target polynucleotide, such as any described above, e.g., CIITA, B2M, or NLRC5, can be knocked down in a cell by transducing a shRNA-expressing virus into the cell. In some embodiments, RNA interference is employed to reduce or inhibit the expression of at least one selected from the group consisting of CIITA, B2M, and NLRC5.

c. Exemplary Target Polynucleotides and Methods for Reducing Expression

### 4) MHC Class I

In certain embodiments, the modification reduces or eliminates, such as knocks out, the expression of MHC class I molecules (e.g., MHC class I genes encoding MHC class I molecules) by targeting the accessory chain B2M. In some embodiments, the modification occurs using a CRISPR/Cas system. By reducing or eliminating, such as knocking out, expression of B2M, surface trafficking of MHC class I molecules is blocked, and such cells exhibit immune tolerance when engrafted into a recipient subject. In some embodiments, the cell is considered hypoimmunogenic, e.g., in a recipient subject or patient upon administration.

In some embodiments, the target polynucleotide sequence provided herein is a variant of B2M. In some embodiments, the target polynucleotide sequence is a homolog of B2M. In some embodiments, the target polynucleotide sequence is an ortholog of B2M.

In some embodiments, decreased or eliminated expression of B2M reduces or eliminates expression of one or more of the following MHC class I molecules—HLA-A, HLA-B, and HLA-C.

In some embodiments, the modified pluripotent stem cells cell comprises a modification targeting the B2M gene. In some embodiments, the modification targeting the B2M gene is by using a targeted nuclease system that comprises a Cas protein or a polynucleotide encoding a Cas protein, and at least one guide ribonucleic acid sequence for specifically targeting the B2M gene. In some embodiments, the at least one guide ribonucleic acid sequence (e.g., gRNA targeting sequence) for specifically targeting the B2M gene is selected from the group consisting of SEQ ID NOS: 81240-85644 of Appendix 2 or Table 15 of WO2016/183041, the disclosure of which is herein incorporated by reference in its entirety.

In some embodiments, an exogenous nucleic acid or transgene encoding a polypeptide as disclosed herein (e.g., a chimeric antigen receptor, CD47, or another tolerogenic factor disclosed herein) is inserted at the B2M gene. Exemplary transgenes for targeted insertion at the B2M locus include any as described herein.

Assays to test whether the B2M gene has been inactivated are known and described herein. In one embodiment, the resulting modification of the B2M gene by PCR and the reduction of HLA-I expression can be assays by flow cytometry, such as by FACS analysis. In another embodiment, B2M protein expression is detected using a Western blot of cells lysates probed with antibodies to the B2M protein. In another embodiment, reverse transcriptase polymerase chain reactions (RT-PCR) are used to confirm the presence of the inactivating modification.

In some embodiments, the technologies disclosed herein modulate (e.g., reduce or eliminate) the expression of MHC-I genes by targeting and modulating (e.g., reducing or eliminating) expression of the NLR family, CARD domain containing 5/NOD27/CLR16.1 (NLRC5). In some embodiments, the modulation occurs using a CRISPR/Cas system. NLRC5 is a critical regulator of MHC-I-mediated immune responses and, similar to CIITA, NLRC5 is highly inducible by IFN-γ and can translocate into the nucleus. NLRC5 activates the promoters of MHC-I genes and induces the transcription of MHC-I as well as related genes involved in MHC-I antigen presentation.

In some embodiments, the target polynucleotide sequence is a variant of NLRC5. In some embodiments, the target polynucleotide sequence is a homolog of NLRC5. In some embodiments, the target polynucleotide sequence is an ortholog of NLRC5.

In some embodiments, the cells outlined herein comprise a genetic modification targeting the NLRC5 gene. In some embodiments, the genetic modification targeting the NLRC5 gene by the rare-cutting endonuclease comprises a Cas protein or a polynucleotide encoding a Cas protein, and at least one guide ribonucleic acid sequence for specifically targeting the NLRC5 gene. In some embodiments, the at least one guide ribonucleic acid sequence for specifically targeting the NLRC5 gene is selected from the group consisting of SEQ ID NOS: 36353-81239 of Appendix 3 or Table 14 of WO2016183041, the disclosure is incorporated by reference in its entirety.

Assays to test whether the NLRC5 gene has been inactivated are known and described herein. In some embodiments, the resulting genetic modification of the NLRC5 gene by PCR and the reduction of HLA-I expression can be assays by FACS analysis. In another embodiment, NLRC5 protein expression is detected using a Western blot of cells lysates probed with antibodies to the NLRC5 protein. In another embodiment, reverse transcriptase polymerase chain reactions (RT-PCR) are used to confirm the presence of the inactivating genetic modification.

In some embodiments, the reduction of the MHC class I expression or function (HLA I when the cells are derived from human cells) in the modified cells can be measured using techniques known in the art; for example, FACS techniques using labeled antibodies that bind the HLA complex; for example, using commercially available HLA-A, B, C antibodies that bind to the alpha chain of the human major histocompatibility HLA Class I antigens. In addition, the cells can be tested to confirm that the HLA I complex is not expressed on the cell surface. This may be assayed by FACS analysis using antibodies to one or more HLA cell surface components as discussed above. In addition to the reduction of HLA I (or MHC class I), the modified pluripotent stem cells provided herein have a reduced susceptibility to macrophage phagocytosis and NK cell killing. Methods to assay for hypoimmunogenic phenotypes of the modified cells are described further below.

### 5) MHC Class II

In certain aspects, the modification reduces or eliminates, such as knocks out, the expression of MHC class II genes by targeting Class II transactivator (CIITA) expression. In some embodiments, the modification occurs using a CRISPR/Cas system. CIITA is a member of the LR or nucleotide binding domain (NBD) leucine-rich repeat (LRR) family of proteins and regulates the transcription of MHC class II by associating with the MHC enhanceosome. By reducing or eliminating, such as knocking out, expression of CIITA, expression of MHC class II molecules is reduced thereby also reducing surface expression. In some cases, such cells exhibit immune tolerance when engrafted into a recipient subject. In some embodiments, the cell is considered hypoimmunogenic, e.g., in a recipient subject or patient upon administration.

In some embodiments, the target polynucleotide sequence is a variant of CIITA. In some embodiments, the target polynucleotide sequence is a homolog of CIITA. In some embodiments, the target polynucleotide sequence is an ortholog of CIITA.

In some embodiments, reduced or eliminated expression of CIITA reduces or eliminates expression of one or more of the following MHC class II are HLA-DP, HLA-DM, HLA-DOA, HLA-DOB, HLA-DQ, and HLA-DR.

In some embodiments, the modified cell comprises a modification targeting the CIITA gene. In some embodiments, the modification targeting the CIITA gene is by a targeted nuclease system that comprises a Cas protein or a polynucleotide encoding a Cas protein, and at least one guide ribonucleic acid sequence for specifically targeting the CIITA gene. In some embodiments, the at least one guide ribonucleic acid sequence (e.g., gRNA targeting sequence) for specifically targeting the CIITA gene is selected from the group consisting of SEQ ID NOS: 5184-36352 of Appendix 1 or Table 12 of WO2016183041, the disclosure is incorporated by reference in its entirety.

In some embodiments, an exogenous nucleic acid or transgene encoding a polypeptide as disclosed herein (e.g., a chimeric antigen receptor, CD47, or another tolerogenic factor disclosed herein) is inserted at the CIITA gene. Exemplary transgenes for targeted insertion at the B2M locus include any as described herein.

Assays to test whether the CIITA gene has been inactivated are known and described herein. In one embodiment, the resulting modification of the CIITA gene by PCR and the reduction of HLA-II expression can be assays by flow cytometry, such as by FACS analysis. In another embodiment, CIITA protein expression is detected using a Western blot of cells lysates probed with antibodies to the CIITA protein. In another embodiment, reverse transcriptase polymerase chain reactions (RT-PCR) are used to confirm the presence of the inactivating modification.

In some embodiments, the reduction of the MHC class II expression or function (HLA II when the cells are derived from human cells) in the modified cells can be measured using techniques known in the art, such as Western blotting using antibodies to the protein, FACS techniques, RT-PCR techniques, etc. In some embodiments, the modified cells can be tested to confirm that the HLA II complex is not expressed on the cell surface. Methods to assess surface expression include methods known in the art (See FIG. 21 of WO2018132783, for example) and generally is done using either Western Blots or FACS analysis based on commercial antibodies that bind to human HLA Class II HLA-DR, DP and most DQ antigens. In addition to the reduction of HLA II (or MHC class II), the modified pluripotent stem cells provided herein have a reduced susceptibility to macrophage phagocytosis and NK cell killing. Methods to assay for hypoimmunogenic phenotypes of the modified cells are described further below.

### 6) CD142

In certain aspects, the modification reduces or eliminates, such as knocks out, the expression of CD142. In some embodiments, the modification occurs using a CRISPR/Cas system. CD142, also known as tissue factor (F3) is a membrane-bound protein that initiates blood coagulation by forming a complex with circulating factor VII or factor VIIa. The CD142 (TF): VIIa complex activates factors IX or X by specific limited proteolysis. CD142 (TF) plays a role in normal hemostasis by initiating the cell-surface assembly and propagation of the coagulation protease cascade. By reducing or eliminating, such as knocking out, expression of CD142, expression of MHC class II molecules is reduced thereby also reducing surface expression. In some cases, such cells exhibit immune tolerance when engrafted into a recipient subject. In some embodiments, the cell is considered hypoimmunogenic, e.g., in a recipient subject or patient upon administration.

In some embodiments, the target polynucleotide sequence is a variant of CD142. In some embodiments, the target polynucleotide sequence is a homolog of CD142. In some embodiments, the target polynucleotide sequence is an ortholog of CD142.

In some embodiments, the modified pluripotent stem cells comprises a modification targeting the CD142 gene. In some embodiments, the modification targeting the CD142 gene is by a targeted nuclease system that comprises a Cas protein or a polynucleotide encoding a Cas protein, and at least one guide ribonucleic acid sequence for specifically targeting the CD142 gene. In some embodiments, the target polynucleotide sequence is CD142 or a variant of CD142. In some embodiments, the target polynucleotide sequence is a homolog of CD142. In some embodiments, the target polynucleotide sequence is an ortholog of CD142.

In some embodiments, the cells outlined herein may comprise a modification targeting the CD142 gene. In some embodiments, the modification targeting the CD142 gene by the rare-cutting endonuclease comprises a Cas protein or a polynucleotide encoding a Cas protein, and at least one guide ribonucleic acid (gRNA) sequence for specifically targeting the CD142 gene. Useful methods for identifying gRNA sequences to target CD142 are described below.

Assays to test whether the CD142 gene has been inactivated are known and described herein. In one embodiment, the resulting modification of the CD142 gene by PCR and the reduction of CD142 expression can be assays by FACS analysis. In another embodiment, CD142 protein expression is detected using a Western blot of cells lysates probed with antibodies to the CD142 protein. In another embodiment, reverse transcriptase polymerase chain reactions (RT-PCR) are used to confirm the presence of the inactivating modification. Useful genomic, polynucleotide and polypeptide information about the human CD142 are provided in, for example, the GeneCard Identifier GC01M094530, HGNC No. 3541, NCBI Gene ID 2152, NCBI RefSeq Nos. NM_001178096.1, NM_001993.4, NP_001171567.1, and NP_001984.1, UniProt No. P13726, and the like.

In some embodiments, an exogenous nucleic acid or transgene encoding a polypeptide as disclosed herein (e.g., a chimeric antigen receptor, CD46, CD59, CD55, or CD47 or another tolerogenic factor disclosed herein) is inserted at the CD142 gene. Exemplary transgenes for targeted insertion at the CD142 locus include any as described herein.

In some embodiments, the reduction of the CD142 expression or function in the modified cells can be measured using techniques known in the art, such as Western blotting using antibodies to the protein, FACS techniques, RT-PCR techniques, etc. In some embodiments, the modified cells can be tested to confirm that CD142 is not expressed on the cell surface. Methods to assess surface expression include methods known in the art (See FIG. 21 of WO2018132783, for example) and generally is done using either Western Blots or FACS analysis based on commercial antibodies that bind to human CD142. In addition to the reduction of CD142, the modified cells provided herein have a reduced susceptibility to IBMIR. Methods to assay for hypoimmunogenic phenotypes of the modified cells are described further below.

In some embodiments, the modification that reduces CD142 expression reduces CD142 mRNA expression. In some embodiments, the reduced mRNA expression of CD142 is relative to an unmodified or wild-type cell of the same cell type that does not comprise the modification. In some embodiments, the mRNA expression of CD142 is reduced by more than about 5%, such as reduced by more than about any of 10%, 20%, 30%, 40%, 50%, 60%, 70%, 80%, 90%, or more. In some embodiments, the mRNA expression of CD142 is reduced by up to about 100%, such as reduced by up to about any of 90%, 80%, 70%, 60%, 50%, 40%, 30%, 20%, 10%, 5%, or less. In some embodiments, the mRNA expression of CD142 is reduced by any of about 5%, 10%, 20%, 30%, 40%, 50%, 60%, 70%, 80%, 90%, or 100%. In some embodiments, the mRNA expression of CD142 is eliminated (e.g., 0% expression of CD142 mRNA). In some embodiments, the modification that reduces CD142 mRNA expression eliminates CD142 gene activity.

In some embodiments, the modification that reduces CD142 expression reduces CD142 protein expression. In some embodiments, the reduced protein expression of CD142 is relative to an unmodified or wild-type cell of the same cell type that does not comprise the modification. In some embodiments, the protein expression of CD142 is reduced by more than about 5%, such as reduced by more than about any of 10%, 20%, 30%, 40%, 50%, 60%, 70%, 80%, 90%, or more. In some embodiments, the protein expression of CD142 is reduced by up to about 100%, such as reduced by up to about any of 90%, 80%, 70%, 60%, 50%, 40%, 30%, 20%, 10%, 5%, or less. In some embodiments, the protein expression of CD142 is reduced by any of about 5%, 10%, 20%, 30%, 40%, 50%, 60%, 70%, 80%, 90%, or 100%. In some embodiments, the protein expression of CD142 is eliminated (e.g., 0% expression of CD142 protein). In some embodiments, the modification that reduces CD142 protein expression eliminates CD142 gene activity.

In some embodiments, the modification that reduces CD142 expression comprises inactivation or disruption of the CD142 gene. In some embodiments, the modification that reduces CD142 expression comprises inactivation or disruption of one allele of the CD142 gene. In some embodiments, the modification that reduces CD142 expression comprises inactivation or disruption comprises inactivation or disruption of both alleles of the CD142 gene.

In some embodiments, the modification comprises inactivation or disruption of one or more CD142 coding sequences in the cell. In some embodiments, the modification comprises inactivation or disruption of all CD142 coding sequences in the cell. In some embodiments, the modification comprises inactivation or disruption comprises an indel in the CD142 gene. In some embodiments, the modification is a frameshift mutation of genomic DNA of the CD142 gene. In some embodiments, the modification is a deletion of genomic DNA of the CD142 gene. In some embodiments, the modification is a deletion of a contiguous stretch of genomic DNA of the CD142 gene.

Exemplary guide target sequences for CD142 are known, for example:

| | |
|---|---|
| F3 CRISPR Guide RNA | TTCCCTCCCGAACAGTTAAC |
| or crRNA 1 | (SEQ ID NO: 22) |
| F3 CRISPR Guide RNA | CCGGGCTGTCTGTACTCTTC |
| or crRNA 2 | (SEQ ID NO: 23) |
| F3 CRISPR Guide RNA | CCTCACCGACGAGATTGTGA |
| or crRNA 3 | (SEQ ID NO: 24) |
| F3 CRISPR Guide RNA | ATGTGACCGTAGAAGATGAA |
| or crRNA 4 | (SEQ ID NO: 25) |
| F3 CRISPR Guide RNA | TAAAGTCCGTTCATCTTCTA |
| or crRNA 5 | (SEQ ID NO: 26) |
| F3 CRISPR Guide RNA | CACATCCTTCACAATCTCGT |
| or crRNA 6 | (SEQ ID NO: 27) |

### 2. Overexpression of Polynucleotides

In some embodiments, the modified pluripotent stem cells provided herein are genetically modified, such as by introduction of one or more modifications into a cell to overexpress a desired polynucleotide in the cell. In some embodiments, the cell to be modified is an unmodified cell that has not previously been introduced with the one or more modifications. In some embodiments, the modified pluripotent stem cells provided herein are genetically modified to include one or more exogenous polynucleotides encoding an exogenous protein (also interchangeably used with the term “transgene”). As described, in some embodiments, the cells are modified to increase expression of certain genes that are tolerogenic (e.g., immune) factors that affect immune recognition and tolerance in a recipient. In some embodiments, the provided modified cells, such as T cells or NK cells, also express a chimeric antigen receptor (CAR). The one or more polynucleotides, e.g., exogenous polynucleotides, may be expressed (e.g. overexpressed) in the modified pluripotent stem cells together with one or more genetic modifications to reduce expression of a target polynucleotide described above, such as an MHC class I and/or MHC class II molecule or CD142. In some embodiments, the provided modified pluripotent stem cells do not trigger or activate an immune response upon administration to a recipient subject.

In some embodiments, the modified pluripotent stem cell includes 1, 2, 3, 4, 5, 6, 7, 8, 9, 10 or more different overexpressed polynucleotides. In some embodiments, the overexpressed polynucleotide is an exogenous polynucleotide. In some embodiments, the modified pluripotent stem cell includes 1, 2, 3, 4, 5, 6, 7, 8, 9, 10 or more different exogenous polynucleotides. In some embodiments, the overexpressed polynucleotide is an exogenous polynucleotide that is expressed episomally in the cells. In some embodiments, the overexpressed polynucleotide is an exogenous polynucleotide that is inserted or integrated into one or more genomic loci of the modified cell.

In some embodiments, expression of a polynucleotide is increased, i.e., the polynucleotide is overexpressed, using a fusion protein containing a DNA-targeting domain and a transcriptional activator. Targeted methods of increasing expression using transactivator domains are known to a skilled artisan.

In some embodiments, the modified pluripotent stem cell contains one or more exogenous polynucleotides in which the one or more exogenous polynucleotides are inserted or integrated into a genomic locus of the cell by non-targeted insertion methods, such as by transduction with a lentiviral vector. In some embodiments, the one or more exogenous polynucleotides are inserted or integrated into the genome of the cell by targeted insertion methods, such as by using homology directed repair (HDR). Any suitable method can be used to insert the exogenous polynucleotide into the genomic locus of the modified cell by HDR including the gene editing methods described herein (e.g., a CRISPR/Cas system). In some embodiments, the one or more exogenous polynucleotides are inserted into one or more genomic locus, such as any genomic locus described herein (e.g., Table 2). In some embodiments, the exogenous polynucleotides are inserted into the same genomic loci. In some embodiments, the exogenous polynucleotides are inserted into different genomic loci. In some embodiments, the two or more of the exogenous polynucleotides are inserted into the same genomic loci, such as any genomic locus described herein (e.g., Table 2). In some embodiments, two or more exogenous polynucleotides are inserted into a different genomic loci, such as two or more genomic loci as described herein (e.g., Table 2).

Exemplary polynucleotides or overexpression, and methods for overexpressing the same, are described in the following subsections.

d. Target Genes

### 7) Tolerogenic Factor

In some embodiments, expression of a tolerogenic factor is overexpressed or increased in the cell. In some embodiments, the modified pluripotent stem cell includes increased expression, i.e., overexpression, of at least one tolerogenic factor. In some embodiments, the tolerogenic factor is any factor that promotes or contributes to promoting or inducing tolerance to the modified cell by the immune system (e.g., innate or adaptive immune system). In some embodiments, the tolerogenic factor is DUX4, B2M-HLA-E, CD16, CD52, CD47, CD27, CD200, HLA-C, HLA-E, HLA-E heavy chain, HLA-G, PD-L1, IDO1, CTLA4-Ig, Cl-Inhibitor, IL-10, IL-35, FASL, CCL21, MFGE8, SERPINB9, CD35, IL-39, CD16 Fc Receptor, IL15-RF, and H2-M3. In some embodiments, the tolerogenic factor is CD47, PD-L1, HLA-E or HLA-G, CCL21, FasL, Serpinb9, CD200 or Mfge8, or any combination thereof. In some embodiments, the one or more tolerogenic factors are selected from the group consisting of CD16, CD24, CD35, CD39, CD46, CD47, CD52, CD55, CD59, CD64, CD200, CCL22, CTLA4-Ig, C1 inhibitor, FASL, IDO1, HLA-C, HLA-E, HLA-E heavy chain, HLA-G, IL-10, IL-35, PD-L1, SERPINB9, CCL21, MFGE8, DUX4, B2M-HLA-E, CD27, IL-39, CD16 Fc Receptor, IL15-RF, H2-M3 (HLA-G), A20/TNFAIP3, CR1, HLA-F, and MANF. In some embodiments, the cell includes at least one exogenous polynucleotide that includes a polynucleotide that encodes for a tolerogenic factor. For instance, in some embodiments, at least one of the exogenous polynucleotides is a polynucleotide that encodes CD47. Provided herein are cells that do not trigger or activate an immune response upon administration to a recipient subject. As described above, in some embodiments, the cells are modified to increase expression of genes and tolerogenic (e.g., immune) factors that affect immune recognition and tolerance in a recipient.

In some embodiments, the present disclosure provides a cell or population thereof that has been modified to express the tolerogenic factor (e.g., immunomodulatory polypeptide), such as CD47. In some embodiments, the present disclosure provides a method for altering a cell genome to express the tolerogenic factor (e.g., immunomodulatory polypeptide), such as CD47. In some embodiments, the modified cell expresses an exogenous tolerogenic factor (e.g., immunomodulatory polypeptide), such as an exogenous CD47. In some instances, overexpression or increasing expression of the exogenous polynucleotide is achieved by introducing into the cell (e.g., transducing the cell) within expression vector comprising a nucleotide sequence encoding a human CD47 polypeptide. In some embodiments, the expression vector may be a viral vector, such as a lentiviral vector) or may be a non-viral vector. In some embodiments, the cell is modified to contain one or more exogenous polynucleotides in which at least one of the exogenous polynucleotides includes a polynucleotide that encodes for a tolerogenic factor. In some of any embodiments, the tolerogenic factor is DUX4, B2M-HLA-E, CD16, CD52, CD47, CD27, CD200, HLA-C, HLA-E, HLA-E heavy chain, HLA-G, PD-L1, IDO1, CTLA4-Ig, Cl-Inhibitor, IL-10, IL-35, FASL, CCL21, MFGE8, SERPINB9, CD35, IL-39, CD16 Fc Receptor, IL15-RF, and H2-M3. In some embodiments, the tolerogenic factor is selected from CD47, PD-L1, HLA-E or HLA-G, CCL21, FasL, Serpinb9, CD200 or Mfge8, or any combination thereof (e.g., all thereof). In some embodiments, the one or more tolerogenic factors are selected from the group consisting of CD16, CD24, CD35, CD39, CD46, CD47, CD52, CD55, CD59, CD64, CD200, CCL22, CTLA4-Ig, C1 inhibitor, FASL, IDO1, HLA-C, HLA-E, HLA-E heavy chain, HLA-G, IL-10, IL-35, PD-L1, SERPINB9, CCL21, MFGE8, DUX4, B2M-HLA-E, CD27, IL-39, CD16 Fc Receptor, IL15-RF, H2-M3 (HLA-G), A20/TNFAIP3, CR1, HLA-F, and MANF. For instance, in some embodiments, at least one of the exogenous polynucleotides is a polynucleotide that encodes CD47.

In some embodiments, the tolerogenic factor is CD47. In some embodiments, the modified pluripotent stem cell contains an exogenous polynucleotide that encodes CD47, such as human CD47. In some embodiments, CD47 is overexpressed in the cell. In some embodiments, the expression of CD47 is overexpressed or increased in the modified cell compared to a similar cell of the same cell type that has not been modified with the modification, such as a reference or unmodified cell, e.g. a cell not modified with an exogenous polynucleotide encoding CD47. CD47 is a leukocyte surface antigen and has a role in cell adhesion and modulation of integrins. It is normally expressed on the surface of a cell and signals to circulating macrophages not to eat the cell. Useful genomic, polynucleotide and polypeptide information about human CD47 are provided in, for example, the NP_001768.1, NP_942088.1, NM_001777.3 and NM_198793.2.

In some embodiments, the modified pluripotent stem cell includes increased expression, i.e. overexpression, of at least one tolerogenic factor. In some embodiments, the cell includes at least one exogenous polynucleotide that includes a polynucleotide that encodes for a tolerogenic factor. In some embodiments, tolerogenic factors include DUX4, B2M-HLA-E, CD16, CD52, CD47, CD27, CD200, HLA-C, HLA-E, HLA-E heavy chain, HLA-G, PD-L1, IDO1, CTLA4-Ig, Cl-Inhibitor, IL-10, IL-35, FASL, CCL21, MFGE8, SERPINB9, CD35, IL-39, CD16 Fc Receptor, IL15-RF, and H2-M3, or any combination thereof. In some embodiments, the one or more tolerogenic factors are selected from the group consisting of CD16, CD24, CD35, CD39, CD46, CD47, CD52, CD55, CD59, CD64, CD200, CCL22, CTLA4-Ig, C1 inhibitor, FASL, IDO1, HLA-C, HLA-E, HLA-E heavy chain, HLA-G, IL-10, IL-35, PD-L1, SERPINB9, CCL21, MFGE8, DUX4, B2M-HLA-E, CD27, IL-39, CD16 Fc Receptor, IL15-RF, H2-M3 (HLA-G), A20/TNFAIP3, CR1, HLA-F, and MANF. For instance, in some embodiments, at least one of the overexpressed (e.g., exogenous) polynucleotides is a polynucleotide that encodes CD47.

In some embodiments, the present disclosure provides a cell or population thereof that has been modified to express the tolerogenic factor (e.g., immunomodulatory polypeptide), such as CD47. In some embodiments, the present disclosure provides a method for altering a cell genome to express the tolerogenic factor (e.g., immunomodulatory polypeptide), such as CD47. In some embodiments, the modified pluripotent stem cell expresses an exogenous tolerogenic factor (e.g., immunomodulatory polypeptide), such as an exogenous CD47. In some instances, the cell expresses an expression vector comprising a nucleotide sequence encoding a human CD47 polypeptide.

In some embodiments, the modified pluripotent stem cell contains an overexpressed polynucleotide that encodes CD47, such as human CD47. In some embodiments, the modified pluripotent stem cell contains an exogenous polynucleotide that encodes CD47, such as human CD47. In some embodiments, CD47 is overexpressed in the cell. In some embodiments, the expression of CD47 is increased in the modified pluripotent stem cell compared to a similar reference or unmodified cell (including with any other modifications) except that the reference or unmodified cell does not include the exogenous polynucleotide encoding CD47.

In some embodiments, the cell outlined herein comprises an exogenous nucleotide sequence encoding a CD47 polypeptide has at least 95% sequence identity (e.g., 95%, 96%, 97%, 98%, 99%, or more) to an amino acid sequence as set forth in NCBI Ref. Sequence Nos. NP_001768.1 and NP_942088.1. In some embodiments, the cell outlined herein comprises an exogenous nucleotide sequence encoding a CD47 polypeptide having an amino acid sequence as set forth in NCBI Ref. Sequence Nos. NP_001768.1 and NP_942088.1. In some embodiments, the cell comprises an exogenous nucleotide sequence for CD47 having at least 85% sequence identity (e.g., 85%, 86%, 87%, 88%, 89%, 90%, 91%, 92%, 93%, 94%, 95%, 96%, 97%, 98%, 99%, or more) to the sequence set forth in NCBI Ref. Nos. NM_001777.3 and NM_198793.2. In some embodiments, the cell comprises an exogenous nucleotide sequence for CD47 as set forth in NCBI Ref. Sequence Nos. NM_001777.3 and NM_198793.2.

In some embodiments, the cell outlined herein comprises an exogenous nucleotide sequence encoding a CD47 polypeptide has at least 95% sequence identity (e.g., 95%, 96%, 97%, 98%, 99%, or more) to an amino acid sequence as set forth in NCBI Ref. Sequence Nos. NP_001768.1 and NP_942088.1. In some embodiments, the cell outlined herein comprises an exogenous nucleotide sequence encoding a CD47 polypeptide having an amino acid sequence as set forth in NCBI Ref. Sequence Nos. NP_001768.1 and NP_942088.1. In some embodiments, the cell comprises an exogenous nucleotide sequence for CD47 having at least 85% sequence identity (e.g., 85%, 86%, 87%, 88%, 89%, 90%, 91%, 92%, 93%, 94%, 95%, 96%, 97%, 98%, 99%, or more) to the sequence set forth in NCBI Ref. Nos. NM_001777.3 and NM_198793.2. In some embodiments, the cell comprises an exogenous nucleotide sequence for CD47 as set forth in NCBI Ref. Sequence Nos. NM_001777.3 and NM_198793.2.

In some embodiments, the cell comprises an exogenous CD47 polypeptide having at least 95% sequence identity (e.g., 95%, 96%, 97%, 98%, 99%, or more) to an amino acid sequence as set forth in NCBI Ref. Sequence Nos. NP_001768.1 and NP_942088.1. In some embodiments, the cell outlined herein comprises an exogenous CD47 polypeptide having an amino acid sequence as set forth in NCBI Ref. Sequence Nos. NP_001768.1 and NP_942088.1.

In some embodiments, the cell comprises an overexpressed polynucleotide encoding a CD47 polypeptide having at least 95% sequence identity (e.g., 95%, 96%, 97%, 98%, 99%, or more) to an amino acid sequence as set forth in SEQ ID NO: 1. In some embodiments, the cell comprises an exogenous polynucleotide encoding a CD47 polypeptide having at least 95% sequence identity (e.g., 95%, 96%, 97%, 98%, 99%, or more) to an amino acid sequence as set forth in SEQ ID NO: 1. In some embodiments, the cell comprises an overexpressed polynucleotide encoding a CD47 polypeptide having the amino acid sequence as set forth in SEQ ID NO: 1. In some embodiments, the cell comprises an exogenous polynucleotide encoding a CD47 polypeptide having the amino acid sequence as set forth in SEQ ID NO: 1.

In some embodiments, the cell comprises an overexpressed CD47 polypeptide having at least 95% sequence identity (e.g., 95%, 96%, 97%, 98%, 99%, or more) to an amino acid sequence as set forth in SEQ ID NO: 2. In some embodiments, the cell comprises an exogenous CD47 polypeptide having at least 95% sequence identity (e.g., 95%, 96%, 97%, 98%, 99%, or more) to an amino acid sequence as set forth in SEQ ID NO: 2. In some embodiments, the cell comprises an overexpressed CD47 polypeptide having the amino acid sequence as set forth in SEQ ID NO: 2. In some embodiments, the cell comprises an exogenous CD47 polypeptide having the amino acid sequence as set forth in SEQ ID NO: 2. In some embodiments, the exogenous nucleotide sequence encoding the CD59 polypeptide is operably linked to a sequence encoding a heterologous signal peptide. In some embodiments, an exogenous polynucleotide encoding CD47 is integrated into the genome of the cell by targeted or non-targeted methods of insertion, such as described further below. In some embodiments, targeted insertion is by homology-dependent insertion into a target locus, such as by insertion into any one of the gene loci depicted in Table 2, e.g. a B2M gene or a CIITA gene. In some embodiments, targeted insertion is by homology-independent insertion, such as by insertion into a safe harbor locus. In some cases, the polynucleotide encoding CD47 is inserted into a safe harbor locus, such as but not limited to, a gene locus selected from AAVS1, CCR5, CLYBL, ROSA26, and SHS231. In particular embodiments, the polynucleotide encoding CD47 is inserted into the CCR5 gene locus, the PPP1R12C (also known as AAVS1) gene locus or the CLYBL gene locus.

In some embodiments, all or a functional portion of CD47 can be linked to other components such as a signal peptide, a leader sequence, a secretory signal, a label (e.g., a reporter gene), or any combination thereof. In some embodiments, the nucleic acid sequence encoding a signal peptide of CD47 is replaced with a nucleic acid sequence encoding a signal peptide from a heterologous protein. The heterologous protein can be, for example, CD8α, CD28, tissue plasminogen activator (tPA), growth hormone, granulocyte-macrophage colony stimulating factor (GM-CSF), GM-CSF receptor (GM-CSFRa), or an immunoglobulin (e.g., IgE or IgK). In some embodiments, the signal peptide is a signal peptide from an immunoglobulin (such as IgG heavy chain or IgG-kappa light chain), a cytokine (such as interleukin-2 (IL-2), or CD33), a serum albumin protein (e.g., HSA or albumin), a human azurocidin preprotein signal sequence, a luciferase, a trypsinogen (e.g. chymotrypsinogen or trypsinogen) or other signal peptide able to efficiently express a protein by or on a cell.

In certain embodiments, the exogenous polynucleotide encoding CD47 is operably linked to a promoter.

In some embodiments, the exogenous polynucleotide encoding CD47 is inserted into any one of the gene loci depicted in Table 2. In some cases, the exogenous polynucleotide encoding CD47 is inserted into a safe harbor locus, such as but not limited to, a gene locus selected from AAVS1, CCR5, CLYBL, ROSA26, and SHS231. In particular embodiments, the exogenous polynucleotide encoding CD47 is inserted into the CCR5 gene locus, the PPP1R12C (also known as AAVS1) gene locus or the CLYBL gene locus. In some embodiments, the exogenous polynucleotide encoding CD47 is inserted into a B2M gene locus, a CIITA gene locus, or a CD142 gene locus. In some embodiments, a suitable gene editing system (e.g., CRISPR/Cas system or any of the gene editing systems described herein) is used to facilitate the insertion of a polynucleotide encoding CD47, into a genomic locus of the cell.

In some embodiments, CD47 protein expression is detected using a Western blot of cell lysates probed with antibodies against the CD47 protein. In another embodiment, reverse transcriptase polymerase chain reactions (RT-PCR) are used to confirm the presence of the exogenous CD47 mRNA.

In some embodiments, the modified pluripotent stem cell contains an exogenous polynucleotide that encodes CD200, such as human CD200. In some embodiments, CD200 is overexpressed in the cell. In some embodiments, the expression of CD200 is increased in the modified cell compared to a similar reference or unmodified cell (including with any other modifications) except that the reference or unmodified cell does not include the exogenous polynucleotide encoding CD200. Useful genomic, polynucleotide and polypeptide information about human CD200 are provided in, for example, the GeneCard Identifier GC03P112332, HGNC No. 7203, NCBI Gene ID 4345, Uniprot No. P41217, and NCBI RefSeq Nos. NP_001004196.2, NM_001004196.3, NP_001305757.1, NM_001318828.1, NP_005935.4, NM_005944.6, XP_005247539.1, and XM_005247482.2. In certain embodiments, the polynucleotide encoding CD200 is operably linked to a promoter.

In some embodiments, the polynucleotide encoding CD200 is inserted into any one of the gene loci depicted in Table 2. In some cases, the polynucleotide encoding CD200 is inserted into a safe harbor locus, such as but not limited to, a gene locus selected from AAVS1, CCR5, CLYBL, ROSA26, and SHS231. In particular embodiments, the polynucleotide encoding CD200 is inserted into the CCR5 gene locus, the PPP1R12C (also known as AAVS1) gene locus or the CLYBL gene locus. In some embodiments, the polynucleotide encoding CD200 is inserted into a B2M gene locus, a CIITA gene locus, or a CD142 gene locus. In some embodiments, a suitable gene editing system (e.g., CRISPR/Cas system or any of the gene editing systems described herein) is used to facilitate the insertion of a polynucleotide encoding CD200, into a genomic locus of the cell.

In some embodiments, CD200 protein expression is detected using a Western blot of cell lysates probed with antibodies against the CD200 protein. In another embodiment, reverse transcriptase polymerase chain reactions (RT-PCR) are used to confirm the presence of the exogenous CD200 mRNA.

In some embodiments, the modified pluripotent stem cell contains an exogenous polynucleotide that encodes HLA-E, such as human HLA-E. In some embodiments, HLA-E is overexpressed in the cell. In some embodiments, the expression of HLA-E is increased in the modified pluripotent stem cell compared to a similar reference or unmodified cell (including with any other modifications) except that the reference or unmodified cell does not include the exogenous polynucleotide encoding HLA-E. Useful genomic, polynucleotide and polypeptide information about human HLA-E are provided in, for example, the GeneCard Identifier GC06P047281, HGNC No. 4962, NCBI Gene ID 3133, Uniprot No. P13747, and NCBI RefSeq Nos. NP_005507.3 and NM_005516.5. In certain embodiments, the polynucleotide encoding HLA-E is operably linked to a promoter.

In some embodiments, the polynucleotide encoding HLA-E is inserted into any one of the gene loci depicted in Table 2. In some cases, the polynucleotide encoding HLA-E is inserted into a safe harbor locus, such as but not limited to, a gene locus selected from AAVS1, CCR5, CLYBL, ROSA26, and SHS231. In particular embodiments, the polynucleotide encoding HLA-E is inserted into the CCR5 gene locus, the PPP1R12C (also known as AAVS1) gene locus or the CLYBL gene locus. In some embodiments, the polynucleotide encoding HLA-E is inserted into a B2M gene locus, a CIITA gene locus, or a CD142 gene locus. In some embodiments, a suitable gene editing system (e.g., CRISPR/Cas system or any of the gene editing systems described herein) is used to facilitate the insertion of a polynucleotide encoding HLA-E, into a genomic locus of the cell.

In some embodiments, HLA-E protein expression is detected using a Western blot of cell lysates probed with antibodies against the HLA-E protein. In another embodiment, reverse transcriptase polymerase chain reactions (RT-PCR) are used to confirm the presence of the exogenous HLA-E mRNA.

In some embodiments, the modified pluripotent stem cell contains an exogenous polynucleotide that encodes HLA-G, such as human HLA-G. In some embodiments, HLA-G is overexpressed in the cell. In some embodiments, the expression of HLA-G is increased in the modified pluripotent stem cell compared to a similar reference or unmodified cell (including with any other modifications) except that the reference or unmodified cell does not include the exogenous polynucleotide encoding HLA-G. Useful genomic, polynucleotide and polypeptide information about human HLA-G are provided in, for example, the GeneCard Identifier GC06P047256, HGNC No. 4964, NCBI Gene ID 3135, Uniprot No. P17693, and NCBI RefSeq Nos. NP_002118.1 and NM_002127.5. In certain embodiments, the polynucleotide encoding HLA-G is operably linked to a promoter.

In some embodiments, the polynucleotide encoding HLA-G is inserted into any one of the gene loci depicted in Table 2. In some cases, the polynucleotide encoding HLA-G is inserted into a safe harbor locus, such as but not limited to, a gene locus selected from AAVS1, CCR5, CLYBL, ROSA26, and SHS231. In particular embodiments, the polynucleotide encoding HLA-G is inserted into the CCR5 gene locus, the PPP1R12C (also known as AAVS1) gene locus or the CLYBL gene locus. In some embodiments, the polynucleotide encoding HLA-G is inserted into a B2M gene locus, a CIITA gene locus, or a CD142 gene locus. In some embodiments, a suitable gene editing system (e.g., CRISPR/Cas system or any of the gene editing systems described herein) is used to facilitate the insertion of a polynucleotide encoding HLA-G, into a genomic locus of the cell.

In some embodiments, HLA-G protein expression is detected using a Western blot of cell lysates probed with antibodies against the HLA-G protein. In another embodiment, reverse transcriptase polymerase chain reactions (RT-PCR) are used to confirm the presence of the exogenous HLA-G mRNA.

In some embodiments, the modified pluripotent stem cell contains an exogenous polynucleotide that encodes PD-L1, such as human PD-L1. In some embodiments, PD-L1 is overexpressed in the cell. In some embodiments, the expression of PD-L1 is increased in the modified cell compared to a similar reference or unmodified cell (including with any other modifications) except that the reference or unmodified cell does not include the exogenous polynucleotide encoding PD-L1. Useful genomic, polynucleotide and polypeptide information about human PD-L1 or CD274 are provided in, for example, the GeneCard Identifier GC09P005450, HGNC No. 17635, NCBI Gene ID 29126, Uniprot No. Q9NZQ7, and NCBI RefSeq Nos. NP_001254635.1, NM_001267706.1, NP_054862.1, and NM_014143.3. In certain embodiments, the polynucleotide encoding PD-L1 is operably linked to a promoter.

In some embodiments, the polynucleotide encoding PD-L1 is inserted into any one of the gene loci depicted in Table 2. In some cases, the polynucleotide encoding PD-L1 is inserted into a safe harbor locus, such as but not limited to, a gene locus selected from AAVS1, CCR5, CLYBL, ROSA26, and SHS231. In particular embodiments, the polynucleotide encoding PD-L1 is inserted into the CCR5 gene locus, the PPP1R12C (also known as AAVS1) gene locus or the CLYBL gene locus. In some embodiments, the polynucleotide encoding PD-L1 is inserted into a B2M gene locus, a CIITA gene locus, or a CD142 gene locus. In some embodiments, a suitable gene editing system (e.g., CRISPR/Cas system or any of the gene editing systems described herein) is used to facilitate the insertion of a polynucleotide encoding PD-L1, into a genomic locus of the cell.

In some embodiments, PD-L1 protein expression is detected using a Western blot of cell lysates probed with antibodies against the PD-L1 protein. In another embodiment, reverse transcriptase polymerase chain reactions (RT-PCR) are used to confirm the presence of the exogenous PD-L1 mRNA.

In some embodiments, the modified pluripotent stem cell contains an exogenous polynucleotide that encodes FasL, such as human FasL. In some embodiments, FasL is overexpressed in the cell. In some embodiments, the expression of FasL is increased in the modified pluripotent stem cell compared to a similar reference or unmodified cell (including with any other modifications) except that the reference or unmodified cell does not include the exogenous polynucleotide encoding FasL. Useful genomic, polynucleotide and polypeptide information about human Fas ligand (which is known as FasL, FASLG, CD178, TNFSF6, and the like) are provided in, for example, the GeneCard Identifier GC01P172628, HGNC No. 11936, NCBI Gene ID 356, Uniprot No. P48023, and NCBI RefSeq Nos. NP_000630.1, NM_000639.2, NP_001289675.1, and NM_001302746.1. In certain embodiments, the polynucleotide encoding Fas-L is operably linked to a promoter.

In some embodiments, the polynucleotide encoding Fas-L is inserted into any one of the gene loci depicted in Table 2. In some cases, the polynucleotide encoding Fas-L is inserted into a safe harbor locus, such as but not limited to, a gene locus selected from AAVS1, CCR5, CLYBL, ROSA26, and SHS231. In particular embodiments, the polynucleotide encoding Fas-L is inserted into the CCR5 gene locus, the PPP1R12C (also known as AAVS1) gene locus or the CLYBL gene locus. In some embodiments, the polynucleotide encoding Fas-L is inserted into a B2M gene locus, a CIITA gene locus, or a CD142 gene locus. In some embodiments, a suitable gene editing system (e.g., CRISPR/Cas system or any of the gene editing systems described herein) is used to facilitate the insertion of a polynucleotide encoding Fas-L, into a genomic locus of the cell.

In some embodiments, Fas-L protein expression is detected using a Western blot of cell lysates probed with antibodies against the Fas-L protein. In another embodiment, reverse transcriptase polymerase chain reactions (RT-PCR) are used to confirm the presence of the exogenous Fas-L mRNA.

In some embodiments, the modified pluripotent stem cell contains an exogenous polynucleotide that encodes CCL21, such as human CCL21. In some embodiments, CCL21 is overexpressed in the cell. In some embodiments, the expression of CCL21 is increased in the modified pluripotent stem cell compared to a similar reference or unmodified cell (including with any other modifications) except that the reference or unmodified cell does not include the exogenous polynucleotide encoding CCL21. Useful genomic, polynucleotide and polypeptide information about human CCL21 are provided in, for example, the GeneCard Identifier GC09M034709, HGNC No. 10620, NCBI Gene ID 6366, Uniprot No. 000585, and NCBI RefSeq Nos. NP_002980.1 and NM_002989.3. In certain embodiments, the polynucleotide encoding CCL21 is operably linked to a promoter.

In some embodiments, the polynucleotide encoding CCL21 is inserted into any one of the gene loci depicted in Table 2. In some cases, the polynucleotide encoding CCL21 is inserted into a safe harbor locus, such as but not limited to, a gene locus selected from AAVS1, CCR5, CLYBL, ROSA26, and SHS231. In particular embodiments, the polynucleotide encoding CCL21 is inserted into the CCR5 gene locus, the PPP1R12C (also known as AAVS1) gene locus or the CLYBL gene locus. In some embodiments, the polynucleotide encoding CCL21 is inserted into a B2M gene locus, a CIITA gene locus, or a CD142 gene locus. In some embodiments, a suitable gene editing system (e.g., CRISPR/Cas system or any of the gene editing systems described herein) is used to facilitate the insertion of a polynucleotide encoding CCL21, into a genomic locus of the cell.

In some embodiments, CCL21 protein expression is detected using a Western blot of cell lysates probed with antibodies against the CCL21 protein. In another embodiment, reverse transcriptase polymerase chain reactions (RT-PCR) are used to confirm the presence of the exogenous CCL21 mRNA.

In some embodiments, the modified pluripotent stem cell contains an exogenous polynucleotide that encodes CCL22, such as human CCL22. In some embodiments, CCL22 is overexpressed in the cell. In some embodiments, the expression of CCL22 is increased in the modified cell compared to a similar reference or unmodified cell (including with any other modifications) except that the reference or unmodified cell does not include the exogenous polynucleotide encoding CCL22. Useful genomic, polynucleotide and polypeptide information about human CCL22 are provided in, for example, the GeneCard Identifier GC16P057359, HGNC No. 10621, NCBI Gene ID 6367, Uniprot No. O00626, and NCBI RefSeq Nos. NP_002981.2, NM_002990.4, XP_016879020.1, and XM_017023531.1. In certain embodiments, the polynucleotide encoding CCL22 is operably linked to a promoter.

In some embodiments, the polynucleotide encoding CCL22 is inserted into any one of the gene loci depicted in Table 2. In some cases, the polynucleotide encoding CCL22 is inserted into a safe harbor locus, such as but not limited to, a gene locus selected from AAVS1, CCR5, CLYBL, ROSA26, and SHS231. In particular embodiments, the polynucleotide encoding CCL22 is inserted into the CCR5 gene locus, the PPP1R12C (also known as AAVS1) gene locus or the CLYBL gene locus. In some embodiments, the polynucleotide encoding CCL22 is inserted into a B2M gene locus, a CIITA gene locus, or a CD142 gene locus. In some embodiments, a suitable gene editing system (e.g., CRISPR/Cas system or any of the gene editing systems described herein) is used to facilitate the insertion of a polynucleotide encoding CCL22, into a genomic locus of the cell.

In some embodiments, CCL22 protein expression is detected using a Western blot of cell lysates probed with antibodies against the CCL22 protein. In another embodiment, reverse transcriptase polymerase chain reactions (RT-PCR) are used to confirm the presence of the exogenous CCL22 mRNA.

In some embodiments, the modified pluripotent stem cell contains an exogenous polynucleotide that encodes Mfge8, such as human Mfge8. In some embodiments, Mfge8 is overexpressed in the cell. In some embodiments, the expression of Mfge8 is increased in the modified pluripotent stem cell compared to a similar reference or unmodified cell (including with any other modifications) except that the reference or unmodified cell does not include the exogenous polynucleotide encoding Mfge8. Useful genomic, polynucleotide and polypeptide information about human Mfge8 are provided in, for example, the GeneCard Identifier GC15M088898, HGNC No. 7036, NCBI Gene ID 4240, Uniprot No. Q08431, and NCBI RefSeq Nos. NP_001108086.1, NM_001114614.2, NP_001297248.1, NM_001310319.1, NP_001297249.1, NM_001310320.1, NP_001297250.1, NM_001310321.1, NP_005919.2, and NM_005928.3. In certain embodiments, the polynucleotide encoding Mfge8 is operably linked to a promoter.

In some embodiments, the polynucleotide encoding Mfge8 is inserted into any one of the gene loci depicted in Table 2. In some cases, the polynucleotide encoding Mfge8 is inserted into a safe harbor locus, such as but not limited to, a gene locus selected from AAVS1, CCR5, CLYBL, ROSA26, and SHS231. In particular embodiments, the polynucleotide encoding Mfge8 is inserted into the CCR5 gene locus, the PPP1R12C (also known as AAVS1) gene locus or the CLYBL gene locus. In some embodiments, the polynucleotide encoding Mfge8 is inserted into a B2M gene locus, a CIITA gene locus, a CD142 gene locus. In some embodiments, a suitable gene editing system (e.g., CRISPR/Cas system or any of the gene editing systems described herein) is used to facilitate the insertion of a polynucleotide encoding Mfge8, into a genomic locus of the cell.

In some embodiments, Mfge8 protein expression is detected using a Western blot of cell lysates probed with antibodies against the Mfge8 protein. In another embodiment, reverse transcriptase polymerase chain reactions (RT-PCR) are used to confirm the presence of the exogenous Mfge8 mRNA.

In some embodiments, the modified pluripotent stem cell contains an exogenous polynucleotide that encodes SerpinB9, such as human SerpinB9. In some embodiments, SerpinB9 is overexpressed in the cell. In some embodiments, the expression of SerpinB9 is increased in the modified pluripotent stem cell compared to a similar reference or unmodified cell (including with any other modifications) except that the reference or unmodified cell does not include the exogenous polynucleotide encoding SerpinB9. Useful genomic, polynucleotide and polypeptide information about human SerpinB9 are provided in, for example, the GeneCard Identifier GC06M002887, HGNC No. 8955, NCBI Gene ID 5272, Uniprot No. P50453, and NCBI RefSeq Nos. NP_004146.1, NM_004155.5, XP_005249241.1, and XM_005249184.4. In certain embodiments, the polynucleotide encoding SerpinB9 is operably linked to a promoter.

In some embodiments, the polynucleotide encoding SerpinB9 is inserted into any one of the gene loci depicted in Table 2. In some cases, the polynucleotide encoding SerpinB9 is inserted into a safe harbor locus, such as but not limited to, a gene locus selected from AAVS1, CCR5, CLYBL, ROSA26, and SHS231. In particular embodiments, the polynucleotide encoding SerpinB9 is inserted into the CCR5 gene locus, the PPP1R12C (also known as AAVS1) gene locus or the CLYBL gene locus. In some embodiments, the polynucleotide encoding SerpinB9 is inserted into a B2M gene locus, a CIITA gene locus, or a CD142 gene locus. In some embodiments, a suitable gene editing system (e.g., CRISPR/Cas system or any of the gene editing systems described herein) is used to facilitate the insertion of a polynucleotide encoding SerpinB9, into a genomic locus of the cell.

In some embodiments, SerpinB9 protein expression is detected using a Western blot of cell lysates probed with antibodies against the SerpinB9 protein. In another embodiment, reverse transcriptase polymerase chain reactions (RT-PCR) are used to confirm the presence of the exogenous SerpinB9 mRNA.

In some embodiments, the tolerogenic factor is CD47 and the cell includes an exogenous polynucleotide encoding a CD47 protein. In some embodiments, the cell expresses an exogenous CD47 polypeptide.

In some embodiments, a method disclosed herein comprises administering to a subject in need thereof a CD47-SIRPα blockade agent, wherein the subject was previously administered a population of cells engineered to express an exogenous CD47 polypeptide. In some embodiments, the CD47-SIRPα blockade agent comprises a CD47-binding domain. In some embodiments, the CD47-binding domain comprises signal regulatory protein alpha (SIRPα) or a fragment thereof. In some embodiments, the CD47-SIRPα blockade agent comprises an immunoglobulin G (IgG) Fc domain. In some embodiments, the IgG Fc domain comprises an IgG1 Fc domain. In some embodiments, the IgG1 Fc domain comprises a fragment of a human antibody. In some embodiments, the CD47-SIRPα blockade agent is selected from the group consisting of TTI-621, TTI-622, and ALX148. In some embodiments, the CD47-SIRPα blockade agent is TTI-621, TTI-622, and ALX148. In some embodiments, the CD47-SIRPα blockade agent is TTI-622. In some embodiments, the CD47-SIRPα blockade agent is ALX148. In some embodiments, the IgG Fc domain comprises an IgG4 Fc domain. In some embodiments, the CD47-SIRPα blockade agent is an antibody. In some embodiments, the antibody is selected from the group consisting of MIAP410, B6H12, and Magrolimab. In some embodiments, the antibody is MIAP410. In some embodiments, the antibody is B6H12. In some embodiments, the antibody is Magrolimab. In some embodiments, the antibody is selected from the group consisting of AO-176, IBI188 (letaplimab), STI-6643, and ZL-1201. In some embodiments, the antibody is AO-176 (Arch). In some embodiments, the antibody is IBI188 (letaplimab) (Innovent). In some embodiments, the antibody is STI-6643 (Sorrento). In some embodiments, the antibody is ZL-1201 (Zai).

In some embodiments, useful antibodies or fragments thereof that bind CD47 can be selected from a group that includes magrolimab ((Hu5F9-G4)) (Forty Seven, Inc.; Gilead Sciences, Inc.), urabrelimab, CC-90002 (Celgene; Bristol-Myers Squibb), IBI-188 (Innovent Biologics), IBI-322 (Innovent Biologics), TG-1801 (TG Therapeutics; also known as NI-1701, Novimmune SA), ALX148 (ALX Oncology), TJ011133 (also known as TJC4, I-Mab Biopharma), FA3M3, ZL-1201 (Zai Lab Co., Ltd), AK117 (Akesbio Australia Pty, Ltd.), AO-176 (Arch Oncology), SRF231 (Surface Oncology), GenSci-059 (GeneScience), C47B157 (Janssen Research and Development), C47B161 (Janssen Research and Development), C47B167 (Janssen Research and Development), C47B222 (Janssen Research and Development), C47B227 (Janssen Research and Development), Vx-1004 (Corvus Pharmaceuticals), HMBD004 (Hummingbird Bioscience Pte Ltd), SHR-1603 (Hengrui), AMMS4-G4 (Beijing Institute of Biotechnology), RTX-CD47 (University of Groningen), and IMC-002. (Samsung Biologics; ImmuneOncia Therapeutics). In some embodiments, the antibody or fragment thereof does not compete for CD47 binding with an antibody selected from a group that includes magrolimab, urabrelimab, CC-90002, IBI-188, IBI-322, TG-1801 (NI-1701), ALX148, TJ011133, FA3M3, ZL1201, AK117, AO-176, SRF231, GenSci-059, C47B157, C47B161, C47B167, C47B222, C47B227, Vx-1004, HMBD004, SHR-1603, AMMS4-G4, RTX-CD47, and IMC-002. In some embodiments, the antibody or fragment thereof competes for CD47 binding with an antibody selected from magrolimab, urabrelimab, CC-90002, IBI-188, IBI-322, TG-1801 (NI-1701), ALX148, TJ011133, FA3M3, ZL1201, AK117, AO-176, SRF231, GenSci-059, C47B157, C47B161, C47B167, C47B222, C47B227, Vx-1004, HMBD004, SHR-1603, AMMS4-G4, RTX-CD47, and IMC-002. In some embodiments, the antibody or fragment thereof that binds CD47 is selected from a group that includes a single-chain Fv fragment (scFv) against CD47, a Fab against CD47, a VHH nanobody against CD47, a DARPin against CD47, and variants thereof. In some embodiments, the scFv against CD47, a Fab against CD47, and variants thereof are based on the antigen binding domains of any of the antibodies selected from a group that includes magrolimab, urabrelimab, CC-90002, IBI-188, IBI-322, TG-1801 (NI-1701), ALX148, TJ011133, FA3M3, ZL1201, AK117, AO-176, SRF231, GenSci-059, C47B157, C47B161, C47B167, C47B222, C47B227, Vx-1004, HMBD004, SHR-1603, AMMS4-G4, RTX-CD47, and IMC-002.

In some embodiments, the CD47 antagonist provides CD47 blockade. Methods and agents for CD47 blockade are described in PCT/US2021/054326, which is incorporated by reference in its entirety.

In some embodiments, the tolerogenic factor (e.g., CD47) is overexpressed in the modified PSC relative to the control or wild-type PSC. In some embodiments, the tolerogenic factor (e.g. CD47) is expressed at a first level that is greater than at or about 3-fold, greater than at or about 5-fold, greater than at or about 10-fold, greater than at or about 20-fold, greater than at or about 30-fold, greater than at or about 40-fold, greater than at or about 50-fold, greater than at or about 60-fold, or greater than at or about 70-fold over a second level expressed by the control or wild-type PSC. In some embodiments, the tolerogenic factor (e.g. CD47) is expressed by the modified PSC at greater than at or about 20,000 molecules per cell, at greater than at or about 30,000 molecules per cell, greater than at or about 50,000 molecules per cell, greater than at or about 100,000 molecules per cell, greater than at or about 200,000 molecules per cell, greater than at or about 300,000 molecules per cell, greater than at or about 400,000 molecules per cell, greater than at or about 500,000 molecules per cell, or greater than at or about 600,000 molecules per cell.

In some embodiments, the tolerogenic factor (e.g., CD47) is overexpressed in the modified SC-beta cell relative to the control or wild-type beta cell, such as an unmodified SC-beta cell differentiated from an unmodified PSC that does not contain the modifications. In some embodiments, the tolerogenic factor (e.g. CD47) is expressed at a first level that is greater than at or about 3-fold, greater than at or about 5-fold, greater than at or about 10-fold, greater than at or about 20-fold, greater than at or about 30-fold, greater than at or about 40-fold, greater than at or about 50-fold, greater than at or about 60-fold, or greater than at or about 70-fold over a second level expressed by the control or wild-type beta cell. In some embodiments, the tolerogenic factor (e.g. CD47) is expressed by the modified SC-beta cell at greater than at or about 20,000 molecules per cell, at greater than at or about 30,000 molecules per cell, greater than at or about 50,000 molecules per cell, greater than at or about 100,000 molecules per cell, greater than at or about 200,000 molecules per cell, greater than at or about 300,000 molecules per cell, greater than at or about 400,000 molecules per cell, greater than at or about 500,000 molecules per cell, or greater than at or about 600,000 molecules per cell.

### 8) Complement Inhibitors

In some embodiments, expression of one or more complement inhibitor is increased in the cell. In some embodiments, the one or more complement inhibitor is one or more membrane-bound complement inhibitor. In some embodiments, at least one of the exogenous polynucleotides includes a polynucleotide that encodes for a complement inhibitor. In some embodiments, the one or more complement inhibitor is CD46, CD59, CD55, or CD35 or any combination thereof.

In some embodiments, the one or more complement inhibitor is CD46, CD59, CD55, or any combination thereof. For instance, in some embodiments, at least one of the exogenous polynucleotides is a polynucleotide that encodes one or more complement inhibitors, such as CD46. In some embodiments, the one or more complement inhibitors are CD46 and CD59, or CD46, CD59, and CD55. In some embodiments, expression of CD46 and CD59 or CD46, CD59, and CD55 protects a cell or population thereof from complement-dependent cytotoxicity, including in the presence of antibodies against cell surface antigens expressed by the cell.

In some embodiments, the present disclosure provides a cell or population thereof that has been modified to express the one or more complement inhibitor, such as CD46, CD59, CD55, or any combination thereof. In some embodiments, the one or more complement inhibitor is CD46 and CD59. In some embodiments, the one or more complement inhibitor is CD46, CD59, and CD55. In some embodiments, the present disclosure provides a method for altering a cell genome to express one or more complement inhibitor. In some embodiments, the modified cell expresses one or more exogenous complement inhibitor, such as exogenous CD46 and CD59 or CD46, CD59, and CD55. In some instances, the cell expresses an expression vector comprising a nucleotide sequence encoding a human CD46 polypeptide. In some instances, the cell expresses an expression vector comprising a nucleotide sequence encoding a human CD59 polypeptide. In some instances, the cell expresses an expression vector comprising a nucleotide sequence encoding a human CD55 polypeptide. In some embodiments, the expression vector comprises nucleotide sequences encoding two or more complement inhibitors in any combination. In some embodiments, the expression vector comprises nucleotide sequences encoding CD46 and CD59. In some embodiments, the expression vector comprises nucleotide sequences encoding CD46, CD59, and CD55.

### A) CD46

In some embodiments, the modified pluripotent stem cells contain an overexpressed polynucleotide that encodes CD46, such as human CD46. In some embodiments, the modified pluripotent stem cells contain an exogenous polynucleotide that encodes CD46, such as human CD46. In some embodiments, CD46 is overexpressed in the cell. In some embodiments, the expression of CD46 is increased in the modified pluripotent stem cells compared to a similar reference or unmodified cell (including with any other modifications) except that the reference or unmodified cell does not include the exogenous polynucleotide encoding CD46. CD46 is a membrane-bound complement inhibitor. It acts as a cofactor for complement factor I, a serine protease which protects autologous cells against complement-mediated injury by cleaving C3b and C4b. Useful genomic, polynucleotide and polypeptide information about human CD46 are provided in, for example, the GeneCard Identifier GC01P207752, HGNC No. 6953, NCBI Gene ID 4179, Uniprot No. P15529, and NCBI Ref Seq Nos. NM_002389.4, NM_153826.3, NM_172350.2, NM_172351.2, NM_172352.2 NP_758860.1, NM_172353.2, NM_172359.2, NM_172361.2, NP_002380.3, NP_722548.1, NP_758860.1, NP_758861.1, NP_758862.1, NP_758863.1, NP_758869.1, and NP_758871.1.

In some embodiments, the cell outlined herein comprises an overexpressed nucleotide sequence encoding a CD46 polypeptide has at least 95% sequence identity (e.g., 95%, 96%, 97%, 98%, 99%, or more) to an amino acid sequence as set forth in NCBI Ref. Sequence Nos. NP_002380.3, NP_722548.1, NP_758860.1, NP_758861.1, NP_758862.1, NP_758863.1, NP_758869.1, and NP_758871.1. In some embodiments, the cell outlined herein comprises an overexpressed nucleotide sequence encoding a CD46 polypeptide having an amino acid sequence as set forth in NCBI Ref. Sequence Nos. NP_002380.3, NP_722548.1, NP_758860.1, NP_758861.1, NP_758862.1, NP_758863.1, NP_758869.1, and NP_758871.1. In some embodiments, the cell comprises an overexpressed nucleotide sequence for CD46 having at least 85% sequence identity (e.g., 85%, 86%, 87%, 88%, 89%, 90%, 91%, 92%, 93%, 94%, 95%, 96%, 97%, 98%, 99%, or more) to the sequence set forth in NCBI Ref. Nos. NM_002389.4, NM_153826.3, NM_172350.2, NM_172351.2, NM_172352.2 NP_758860.1, NM_172353.2, NM_172359.2, and NM_172361.2. In some embodiments, the cell comprises an overexpressed nucleotide sequence for CD46 as set forth in NCBI Ref. Sequence Nos. NM_001777.3 and NM_002389.4, NM_153826.3, NM_172350.2, NM_172351.2, NM_172352.2 NP_758860.1, NM_172353.2, NM_172359.2, and NM_172361.2.

In some embodiments, the cell outlined herein comprises an exogenous nucleotide sequence encoding a CD46 polypeptide has at least 95% sequence identity (e.g., 95%, 96%, 97%, 98%, 99%, or more) to an amino acid sequence as set forth in NCBI Ref. Sequence Nos. NP_002380.3, NP_722548.1, NP_758860.1, NP_758861.1, NP_758862.1, NP_758863.1, NP_758869.1, and NP_758871.1. In some embodiments, the cell outlined herein comprises an exogenous nucleotide sequence encoding a CD46 polypeptide having an amino acid sequence as set forth in NCBI Ref. Sequence Nos. NP_002380.3, NP_722548.1, NP_758860.1, NP_758861.1, NP_758862.1, NP_758863.1, NP_758869.1, and NP_758871.1. In some embodiments, the cell comprises an exogenous nucleotide sequence for CD46 having at least 85% sequence identity (e.g., 85%, 86%, 87%, 88%, 89%, 90%, 91%, 92%, 93%, 94%, 95%, 96%, 97%, 98%, 99%, or more) to the sequence set forth in NCBI Ref. Nos. NM_002389.4, NM_153826.3, NM_172350.2, NM_172351.2, NM_172352.2 NP_758860.1, NM_172353.2, NM_172359.2, and NM_172361.2. In some embodiments, the cell comprises an exogenous nucleotide sequence for CD46 as set forth in NCBI Ref. Sequence Nos. NM_001777.3 and NM_002389.4, NM_153826.3, NM_172350.2, NM_172351.2, NM_172352.2 NP_758860.1, NM_172353.2, NM_172359.2, and NM_172361.2.

In some embodiments, the cell comprises an overexpressed CD46 polypeptide having at least 95% sequence identity (e.g., 95%, 96%, 97%, 98%, 99%, or more) to an amino acid sequence as set forth in NCBI Ref. Sequence Nos. NP_722548.1, NP_758860.1, NP_758861.1, NP_758862.1, NP_758863.1, NP_758869.1, and NP_758871.1. In some embodiments, the cell comprises an exogenous CD46 polypeptide having at least 95% sequence identity (e.g., 95%, 96%, 97%, 98%, 99%, or more) to an amino acid sequence as set forth in NCBI Ref. Sequence Nos. NP_722548.1, NP_758860.1, NP_758861.1, NP_758862.1, NP_758863.1, NP_758869.1, and NP_758871.1. In some embodiments, the cell outlined herein comprises an overexpressed CD46 polypeptide having an amino acid sequence as set forth in NCBI Ref. Sequence Nos. NP_722548.1, NP_758860.1, NP_758861.1, NP_758862.1, NP_758863.1, NP_758869.1, and NP_758871.1. In some embodiments, the cell outlined herein comprises an exogenous CD46 polypeptide having an amino acid sequence as set forth in NCBI Ref. Sequence Nos. NP_722548.1, NP_758860.1, NP_758861.1, NP_758862.1, NP_758863.1, NP_758869.1, and NP_758871.1.

In some embodiments, a cell outlined herein comprises an overexpressed nucleotide sequence encoding a CD46 polypeptide that has at least 85% sequence identity (e.g., 85%, 90%, 95%, 96%, 97%, 98%, 99%, or more) to an amino acid sequence as set forth in SEQ ID NO: 4. In some embodiments, a cell outlined herein comprises an exogenous nucleotide sequence encoding a CD46 polypeptide that has at least 85% sequence identity (e.g., 85%, 90%, 95%, 96%, 97%, 98%, 99%, or more) to an amino acid sequence as set forth in SEQ ID NO: 4. In some embodiments, a cell outlined herein comprises an overexpressed nucleotide sequence encoding a CD46 polypeptide comprising an amino acid sequence as set forth in SEQ ID NO: 4. In some embodiments, a cell outlined herein comprises an exogenous nucleotide sequence encoding a CD46 polypeptide comprising an amino acid sequence as set forth in SEQ ID NO: 4.

In some embodiments, a cell outlined herein comprises an exogenous nucleotide sequence encoding a CD46 polypeptide that has at least 85% sequence identity (e.g., 85%, 90%, 95%, 96%, 97%, 98%, 99%, or more) to an amino acid sequence as set forth in SEQ ID NO: 3. In some embodiments, a cell outlined herein comprises an exogenous nucleotide sequence encoding a CD46 polypeptide comprising an amino acid sequence as set forth in SEQ ID NO: 3. In some embodiments, the exogenous nucleotide sequence encoding the CD46 polypeptide is operably linked to a sequence encoding a heterologous signal peptide.

In some embodiments, all or a functional portion of CD46 can be linked to other components such as a signal peptide, a leader sequence, a secretory signal, a label (e.g., a reporter gene), or any combination thereof. In some embodiments, the nucleic acid sequence encoding a signal peptide of CD46 is replaced with a nucleic acid sequence encoding a signal peptide from a heterologous protein. The heterologous protein can be, for example, CD8α, CD28, tissue plasminogen activator (tPA), growth hormone, granulocyte-macrophage colony stimulating factor (GM-CSF), GM-CSF receptor (GM-CSFRa), or an immunoglobulin (e.g., IgE or IgK). In some embodiments, the signal peptide is a signal peptide from an immunoglobulin (such as IgG heavy chain or IgG-kappa light chain), a cytokine (such as interleukin-2 (IL-2), or CD33), a serum albumin protein (e.g. HSA or albumin), a human azurocidin preprotein signal sequence, a luciferase, a trypsinogen (e.g., chymotrypsinogen or trypsinogen) or other signal peptide able to efficiently express a protein by or on a cell.

In certain embodiments, the exogenous polynucleotide encoding CD46 is operably linked to a promoter.

In some embodiments, the polynucleotide encoding CD46 is inserted into any one of the gene loci depicted in Table 2. In some cases, the polynucleotide encoding CD46 is inserted into a safe harbor locus, such as but not limited to, a gene locus selected from AAVS1, CCR5, CLYBL, ROSA26, SHS231. In particular embodiments, the polynucleotide encoding CD46 is inserted into the CCR5 gene locus, the PPP1R12C (also known as AAVS1) gene locus or the CLYBL gene locus. In some embodiments, the polynucleotide encoding CD46 is inserted into a B2M gene locus, a CIITA gene locus, or a CD142 gene locus. In some embodiments, a suitable gene editing system (e.g., CRISPR/Cas system or any of the gene editing systems described herein) is used to facilitate the insertion of a polynucleotide encoding CD46, into a genomic locus of the cell.

In some embodiments, CD46 protein expression is detected using a Western blot of cell lysates probed with antibodies against the CD46 protein. In another embodiment, reverse transcriptase polymerase chain reactions (RT-PCR) are used to confirm the presence of the exogenous CD46 mRNA.

### B) CD59

In some embodiments, the modified pluripotent stem cell contains an overexpressed polynucleotide that encodes CD59, such as human CD59. In some embodiments, the modified pluripotent stem cell contains an exogenous polynucleotide that encodes CD59, such as human CD59. In some embodiments, CD59 is overexpressed in the cell. In some embodiments, the expression of CD59 is increased in the modified pluripotent stem cell compared to a similar reference or unmodified cell (including with any other modifications) except that the reference or unmodified cell does not include the exogenous polynucleotide encoding CD59. CD59 is a membrane-bound complement inhibitor. More specifically, CD59 is an inhibitor of complement membrane attack complex (MAC) activity. CD59 acts by binding to the C8 and/or C9 complements of the assembling MAC, thereby preventing incorporation of the multiple copies of C9 required for complete formation of the osmolytic pore. Useful genomic, polynucleotide and polypeptide information about human CD59 are provided in, for example, the GeneCard Identifier GC11M033704, HGNC No. 1689, NCBI Gene ID 966, Uniprot No. P13987, and NCBI RefSeq Nos. NP_000602.1, NM_000611.5, NP_001120695.1, NM_001127223.1, NP_001120697.1, NM_001127225.1, NP_001120698.1, NM_001127226.1, NP_001120699.1, NM_001127227.1, NP_976074.1, NM_203329.2, NP_976075.1, NM_203330.2, NP_976076.1, and NM_203331.2.

In some embodiments, the cell outlined herein comprises an overexpressed nucleotide sequence encoding a CD59 polypeptide has at least 95% sequence identity (e.g., 95%, 96%, 97%, 98%, 99%, or more) to an amino acid sequence as set forth in NCBI Ref. Sequence Nos. NP_000602.1, NP_001120695.1, NP_001120697.1, NP_001120698.1, NP_001120699.1, NP_976074.1, NP_976075.1, and NP_976076.1. In some embodiments, the cell outlined herein comprises an overexpressed nucleotide sequence encoding a CD59 polypeptide having an amino acid sequence as set forth in NCBI Ref. Sequence Nos. NP_000602.1, NP_001120695.1, NP_001120697.1, NP_001120698.1, NP_001120699.1, NP_976074.1, NP_976075.1, and NP_976076.1. In some embodiments, the cell comprises an overexpressed nucleotide sequence for CD59 having at least 85% sequence identity (e.g., 85%, 86%, 87%, 88%, 89%, 90%, 91%, 92%, 93%, 94%, 95%, 96%, 97%, 98%, 99%, or more) to the sequence set forth in NCBI Ref. Nos. NM_000611.5, NM_001127223.1, NM_001127225.1, NM_001127226.1, NM_001127227.1, NM_203329.2, NM_203330.2, and NM_203331.2. In some embodiments, the cell comprises an overexpressed nucleotide sequence for CD59 as set forth in NCBI Ref. Sequence Nos. NM_000611.5, NM_001127223.1, NM_001127225.1, NM_001127226.1, NM_001127227.1, NM_203329.2, NM_203330.2, and NM_203331.2.

In some embodiments, the cell outlined herein comprises an overexpressed nucleotide sequence encoding a CD59 polypeptide has at least 95% sequence identity (e.g., 95%, 96%, 97%, 98%, 99%, or more) to an amino acid sequence as set forth in NCBI Ref. Sequence Nos. NP_000602.1, NP_001120695.1, NP_001120697.1, NP_001120698.1, NP_001120699.1, NP_976074.1, NP_976075.1, and NP_976076.1. In some embodiments, the cell outlined herein comprises an exogenous nucleotide sequence encoding a CD59 polypeptide has at least 95% sequence identity (e.g., 95%, 96%, 97%, 98%, 99%, or more) to an amino acid sequence as set forth in NCBI Ref. Sequence Nos. NP_000602.1, NP_001120695.1, NP_001120697.1, NP_001120698.1, NP_001120699.1, NP_976074.1, NP_976075.1, and NP_976076.1. In some embodiments, the cell outlined herein comprises an overexpressed nucleotide sequence encoding a CD59 polypeptide having an amino acid sequence as set forth in NCBI Ref. Sequence Nos. NP_000602.1, NP_001120695.1, NP_001120697.1, NP_001120698.1, NP_001120699.1, NP_976074.1, NP_976075.1, and NP_976076.1. In some embodiments, the cell outlined herein comprises an exogenous nucleotide sequence encoding a CD59 polypeptide having an amino acid sequence as set forth in NCBI Ref. Sequence Nos. NP_000602.1, NP_001120695.1, NP_001120697.1, NP_001120698.1, NP_001120699.1, NP_976074.1, NP_976075.1, and NP_976076.1. In some embodiments, the cell comprises an overexpressed nucleotide sequence for CD59 having at least 85% sequence identity (e.g., 85%, 86%, 87%, 88%, 89%, 90%, 91%, 92%, 93%, 94%, 95%, 96%, 97%, 98%, 99%, or more) to the sequence set forth in NCBI Ref. Nos. NM_000611.5, NM_001127223.1, NM_001127225.1, NM_001127226.1, NM_001127227.1, NM_203329.2, NM_203330.2, and NM_203331.2. In some embodiments, the cell comprises an exogenous nucleotide sequence for CD59 having at least 85% sequence identity (e.g., 85%, 86%, 87%, 88%, 89%, 90%, 91%, 92%, 93%, 94%, 95%, 96%, 97%, 98%, 99%, or more) to the sequence set forth in NCBI Ref. Nos. NM_000611.5, NM_001127223.1, NM_001127225.1, NM_001127226.1, NM_001127227.1, NM_203329.2, NM_203330.2, and NM_203331.2. In some embodiments, the cell comprises an overexpressed nucleotide sequence for CD59 as set forth in NCBI Ref. Sequence Nos. NM_000611.5, NM_001127223.1, NM_001127225.1, NM_001127226.1, NM_001127227.1, NM_203329.2, NM_203330.2, and NM_203331.2. In some embodiments, the cell comprises an exogenous nucleotide sequence for CD59 as set forth in NCBI Ref. Sequence Nos. NM_000611.5, NM_001127223.1, NM_001127225.1, NM_001127226.1, NM_001127227.1, NM_203329.2, NM_203330.2, and NM_203331.2.

In some embodiments, the cell comprises an overexpressed CD59 polypeptide having at least 95% sequence identity (e.g., 95%, 96%, 97%, 98%, 99%, or more) to an amino acid sequence as set forth in NCBI Ref. Sequence Nos. NP_000602.1, NP_001120695.1, NP_001120697.1, NP_001120698.1, NP_001120699.1, NP_976074.1, NP_976075.1, and NP_976076.1. In some embodiments, the cell comprises an exogenous CD59 polypeptide having at least 95% sequence identity (e.g., 95%, 96%, 97%, 98%, 99%, or more) to an amino acid sequence as set forth in NCBI Ref. Sequence Nos. NP_000602.1, NP_001120695.1, NP_001120697.1, NP_001120698.1, NP_001120699.1, NP_976074.1, NP_976075.1, and NP_976076.1. In some embodiments, the cell outlined herein comprises an overexpressed CD59 polypeptide having an amino acid sequence as set forth in NCBI Ref. Sequence Nos. NP_000602.1, NP_001120695.1, NP_001120697.1, NP_001120698.1, NP_001120699.1, NP_976074.1, NP_976075.1, and NP_976076.1. In some embodiments, the cell outlined herein comprises an exogenous CD59 polypeptide having an amino acid sequence as set forth in NCBI Ref. Sequence Nos. NP_000602.1, NP_001120695.1, NP_001120697.1, NP_001120698.1, NP_001120699.1, NP_976074.1, NP_976075.1, and NP_976076.1.

In some embodiments, a cell outlined herein comprises an overexpressed nucleotide sequence encoding a CD59 polypeptide that has at least 85% sequence identity (e.g., 85%, 90%, 95%, 96%, 97%, 98%, 99%, or more) to an amino acid sequence as set forth in SEQ ID NO: 6. In some embodiments, a cell outlined herein comprises an exogenous nucleotide sequence encoding a CD59 polypeptide that has at least 85% sequence identity (e.g., 85%, 90%, 95%, 96%, 97%, 98%, 99%, or more) to an amino acid sequence as set forth in SEQ ID NO: 6. In some embodiments, a cell outlined herein comprises an overexpressed nucleotide sequence encoding a CD59 polypeptide comprising an amino acid sequence as set forth in SEQ ID NO: 6. In some embodiments, a cell outlined herein comprises an exogenous nucleotide sequence encoding a CD59 polypeptide comprising an amino acid sequence as set forth in SEQ ID NO: 6.

In some embodiments, a cell outlined herein comprises an overexpressed nucleotide sequence encoding a CD59 polypeptide that has at least 85% sequence identity (e.g., 85%, 90%, 95%, 96%, 97%, 98%, 99%, or more) to an amino acid sequence as set forth in SEQ ID NO: 5. In some embodiments, a cell outlined herein comprises an exogenous nucleotide sequence encoding a CD59 polypeptide that has at least 85% sequence identity (e.g., 85%, 90%, 95%, 96%, 97%, 98%, 99%, or more) to an amino acid sequence as set forth in SEQ ID NO: 5. In some embodiments, a cell outlined herein comprises an overexpressed nucleotide sequence encoding a CD59 polypeptide comprising an amino acid sequence as set forth in SEQ ID NO: 5. In some embodiments, a cell outlined herein comprises an exogenous nucleotide sequence encoding a CD59 polypeptide comprising an amino acid sequence as set forth in SEQ ID NO: 5. In some embodiments, the exogenous nucleotide sequence encoding the CD59 polypeptide is operably linked to a sequence encoding a heterologous signal peptide.

In some embodiments, all or a functional portion of CD59 can be linked to other components such as a signal peptide, a leader sequence, a secretory signal, a label (e.g., a reporter gene), or any combination thereof. In some embodiments, the nucleic acid sequence encoding a signal peptide of CD59 is replaced with a nucleic acid sequence encoding a signal peptide from a heterologous protein. The heterologous protein can be, for example, CD8α, CD28, tissue plasminogen activator (tPA), growth hormone, granulocyte-macrophage colony stimulating factor (GM-CSF), GM-CSF receptor (GM-CSFRa), or an immunoglobulin (e.g., IgE or IgK). In some embodiments, the signal peptide is a signal peptide from an immunoglobulin (such as IgG heavy chain or IgG-kappa light chain), a cytokine (such as interleukin-2 (IL-2), or CD33), a serum albumin protein (e.g., HSA or albumin), a human azurocidin preprotein signal sequence, a luciferase, a trypsinogen (e.g. chymotrypsinogen or trypsinogen) or other signal peptide able to efficiently express a protein by or on a cell.

In certain embodiments, the exogenous polynucleotide encoding CD59 is operably linked to a promoter.

In some embodiments, the polynucleotide encoding CD59 is inserted into any one of the gene loci depicted in Table 2. In some cases, the polynucleotide encoding CD59 is inserted into a safe harbor locus, such as but not limited to, a gene locus selected from AAVS1, CCR5, CLYBL, ROSA26, and SHS231. In particular embodiments, the polynucleotide encoding CD59 is inserted into the CCR5 gene locus, the PPP1R12C (also known as AAVS1) gene locus or the CLYBL gene locus. In some embodiments, the polynucleotide encoding CD59 is inserted into a B2M gene locus, a CIITA gene locus, or a CD142 gene locus. In some embodiments, a suitable gene editing system (e.g., CRISPR/Cas system or any of the gene editing systems described herein) is used to facilitate the insertion of a polynucleotide encoding CD59, into a genomic locus of the cell.

In some embodiments, CD59 protein expression is detected using a Western blot of cell lysates probed with antibodies against the CD59 protein. In another embodiment, reverse transcriptase polymerase chain reactions (RT-PCR) are used to confirm the presence of the exogenous CD59 mRNA.

### C) CD55

In some embodiments, the modified pluripotent stem cell contains an overexpressed polynucleotide that encodes CD55, such as human CD55. In some embodiments, the modified pluripotent stem cell contains an exogenous polynucleotide that encodes CD55, such as human CD55. In some embodiments, CD55 is overexpressed in the cell. In some embodiments, the expression of CD55 is increased in the modified pluripotent stem cell compared to a similar reference or unmodified cell (including with any other modifications) except that the reference or unmodified cell does not include the exogenous polynucleotide encoding CD55. CD55 is a membrane-bound complement inhibitor. In some embodiments, interaction of CD55 with cell-associated C4b and C3b polypeptides interferes with their ability to catalyze the conversion of C2 and factor B to enzymatically active C2a and Bb and thereby prevents the formation of C4b2a and C3bBb, the amplification convertases of the complement cascade. In some embodiments, CD55 inhibits complement activation by destabilizing and preventing the formation of C3 and C5 convertases. Useful genomic, polynucleotide and polypeptide information about human CD55 (also known as complement decay-accelerating factor) are provided in, for example, the GeneCard Identifier GC01P207321, HGNC No. 2665, NCBI Gene ID 1604, Uniprot No. P08174, and NCBI RefSeq Nos. NM_000574.4, NM_001114752.2, NM_001300903.1, NM_001300904.1, NP_000565.1, NP_001108224.1, NP_001287832.1, and NP_001287833.1.

In some embodiments, the cell outlined herein comprises an overexpressed nucleotide sequence encoding a CD55 polypeptide that has at least 95% sequence identity (e.g., 95%, 96%, 97%, 98%, 99%, or more) to an amino acid sequence as set forth in NCBI Ref. Sequence Nos. NP_000565.1, NP_001108224.1, NP_001287832.1, and NP_001287833.1. In some embodiments, the cell outlined herein comprises an overexpressed nucleotide sequence encoding a CD55 polypeptide having an amino acid sequence as set forth in NCBI Ref. Sequence Nos. NP_000565.1, NP_001108224.1, NP_001287832.1, and NP_001287833.1. In some embodiments, the cell comprises an overexpressed nucleotide sequence for CD55 having at least 85% sequence identity (e.g., 85%, 86%, 87%, 88%, 89%, 90%, 91%, 92%, 93%, 94%, 95%, 96%, 97%, 98%, 99%, or more) to the sequence set forth in NCBI Ref. Nos. NM_001777.3 and NM_198793.2. In some embodiments, the cell comprises an overexpressed nucleotide sequence for CD55 as set forth in NCBI Ref. Sequence Nos. NM_000574.4, NM_001114752.2, NM_001300903.1, and NM_001300904.1.

In some embodiments, the cell outlined herein comprises an exogenous nucleotide sequence encoding a CD55 polypeptide that has at least 95% sequence identity (e.g., 95%, 96%, 97%, 98%, 99%, or more) to an amino acid sequence as set forth in NCBI Ref. Sequence Nos. NP_000565.1, NP_001108224.1, NP_001287832.1, and NP_001287833.1. In some embodiments, the cell outlined herein comprises an exogenous nucleotide sequence encoding a CD55 polypeptide having an amino acid sequence as set forth in NCBI Ref. Sequence Nos. NP_000565.1, NP_001108224.1, NP_001287832.1, and NP_001287833.1. In some embodiments, the cell comprises an exogenous nucleotide sequence for CD55 having at least 85% sequence identity (e.g., 85%, 86%, 87%, 88%, 89%, 90%, 91%, 92%, 93%, 94%, 95%, 96%, 97%, 98%, 99%, or more) to the sequence set forth in NCBI Ref. Nos. NM_001777.3 and NM_198793.2. In some embodiments, the cell comprises an exogenous nucleotide sequence for CD55 as set forth in NCBI Ref. Sequence Nos. NM_000574.4, NM_001114752.2, NM_001300903.1, and NM_001300904.1.

In some embodiments, the cell comprises an overexpressed CD55 polypeptide having at least 95% sequence identity (e.g., 95%, 96%, 97%, 98%, 99%, or more) to an amino acid sequence as set forth in NCBI Ref. Sequence Nos. NP_000565.1, NP_001108224.1, NP_001287832.1, and NP_001287833.1. In some embodiments, the cell comprises an exogenous CD55 polypeptide having at least 95% sequence identity (e.g., 95%, 96%, 97%, 98%, 99%, or more) to an amino acid sequence as set forth in NCBI Ref. Sequence Nos. NP_000565.1, NP_001108224.1, NP_001287832.1, and NP_001287833.1. In some embodiments, the cell outlined herein comprises an overexpressed CD55 polypeptide having an amino acid sequence as set forth in NCBI Ref. Sequence Nos. NP_000565.1, NP_001108224.1, NP_001287832.1, and NP_001287833.1. In some embodiments, the cell outlined herein comprises an exogenous CD55 polypeptide having an amino acid sequence as set forth in NCBI Ref. Sequence Nos. NP_000565.1, NP_001108224.1, NP_001287832.1, and NP_001287833.1.

In some embodiments, a cell outlined herein comprises an overexpressed nucleotide sequence encoding a CD55 polypeptide that has at least 85% sequence identity (e.g., 85%, 90%, 95%, 96%, 97%, 98%, 99%, or more) to an amino acid sequence as set forth in SEQ ID NO: 9. In some embodiments, a cell outlined herein comprises an exogenous nucleotide sequence encoding a CD55 polypeptide that has at least 85% sequence identity (e.g., 85%, 90%, 95%, 96%, 97%, 98%, 99%, or more) to an amino acid sequence as set forth in SEQ ID NO: 9. In some embodiments, a cell outlined herein comprises an overexpressed nucleotide sequence encoding a CD55 polypeptide comprising an amino acid sequence as set forth in SEQ ID NO: 9. In some embodiments, a cell outlined herein comprises an exogenous nucleotide sequence encoding a CD55 polypeptide comprising an amino acid sequence as set forth in SEQ ID NO: 9.

In some embodiments, a cell outlined herein comprises an overexpressed nucleotide sequence encoding a CD55 polypeptide that has at least 85% sequence identity (e.g., 85%, 90%, 95%, 96%, 97%, 98%, 99%, or more) to an amino acid sequence as set forth in SEQ ID NO: 8. In some embodiments, a cell outlined herein comprises an exogenous nucleotide sequence encoding a CD55 polypeptide that has at least 85% sequence identity (e.g., 85%, 90%, 95%, 96%, 97%, 98%, 99%, or more) to an amino acid sequence as set forth in SEQ ID NO: 8. In some embodiments, a cell outlined herein comprises an overexpressed nucleotide sequence encoding a CD55 polypeptide comprising an amino acid sequence as set forth in SEQ ID NO: 8. In some embodiments, a cell outlined herein comprises an exogenous nucleotide sequence encoding a CD55 polypeptide comprising an amino acid sequence as set forth in SEQ ID NO: 8. In some embodiments, the exogenous nucleotide sequence encoding the CD59 polypeptide is operably linked to a sequence encoding a heterologous signal peptide.

In some embodiments, all or a functional portion of CD55 can be linked to other components such as a signal peptide, a leader sequence, a secretory signal, a label (e.g., a reporter gene), or any combination thereof. In some embodiments, the nucleic acid sequence encoding a signal peptide of CD55 is replaced with a nucleic acid sequence encoding a signal peptide from a heterologous protein. The heterologous protein can be, for example, CD8α, CD28, tissue plasminogen activator (tPA), growth hormone, granulocyte-macrophage colony stimulating factor (GM-CSF), GM-CSF receptor (GM-CSFRa), or an immunoglobulin (e.g., IgE or IgK). In some embodiments, the signal peptide is a signal peptide from an immunoglobulin (such as IgG heavy chain or IgG-kappa light chain), a cytokine (such as interleukin-2 (IL-2), or CD33), a serum albumin protein (e.g. HSA or albumin), a human azurocidin preprotein signal sequence, a luciferase, a trypsinogen (e.g., chymotrypsinogen or trypsinogen) or other signal peptide able to efficiently express a protein by or on a cell.

In certain embodiments, the exogenous polynucleotide encoding CD55 is operably linked to a promoter.

In some embodiments, the polynucleotide encoding CD55 is inserted into any one of the gene loci depicted in Table 2. In some cases, the polynucleotide encoding CD55 is inserted into a safe harbor locus, such as but not limited to, a gene locus selected from AAVS1, CCR5, CLYBL, ROSA26, and SHS231. In particular embodiments, the polynucleotide encoding CD55 is inserted into the CCR5 gene locus, the PPP1R12C (also known as AAVS1) gene locus or the CLYBL gene locus. In some embodiments, the polynucleotide encoding CD55 is inserted into a B2M gene locus, a CIITA gene locus, or a CD142 gene locus. In some embodiments, a suitable gene editing system (e.g., CRISPR/Cas system or any of the gene editing systems described herein) is used to facilitate the insertion of a polynucleotide encoding CD55, into a genomic locus of the cell.

In some embodiments, CD55 protein expression is detected using a Western blot of cell lysates probed with antibodies against the CD55 protein. In another embodiment, reverse transcriptase polymerase chain reactions (RT-PCR) are used to confirm the presence of the exogenous CD55 mRNA.

### D) Combinations of Complement Inhibitors

In some embodiments, the cell comprises increased expression of none, one, two, or more complement inhibitors selected from the group consisting of CD46, CD59, and CD55, in any combination.

In some embodiments, the modified pluripotent stem cell contains an overexpressed polynucleotide that encodes CD46, such as any described above, and an overexpressed polynucleotide that encodes CD59, such as any described above.

In some embodiments, the modified pluripotent stem cell contains an exogenous polynucleotide that encodes CD46, such as any described above, and an exogenous polynucleotide that encodes CD59, such as any described above.

In some embodiments, the modified cell (comprising one or more modifications that increase expression of CD46 and CD59) comprises increased expression of CD46 and CD59 relative to a cell that does not comprise the modifications (e.g., relative to endogenous expression of CD46 and CD59). In some embodiments, the modified pluripotent stem cell comprises between 1.5-fold and 2-fold, between 2-fold and 3-fold, between 3-fold and 4-fold, between 4-fold and 5-fold, between 5-fold and 10-fold, between 10-fold and 15-fold, between 15-fold and 20-fold, between 20-fold and 40-fold, between 40-fold and 60-fold, between 60-fold and 80-fold, between 80-fold and 100-fold, or between 100-fold and 200-fold increased expression of CD46 and CD59 compared to a cell that does not have the modifications (e.g., compared to endogenous expression of CD46 and CD59). In some embodiments, the cell without the modification(s) does not have endogenous expression of CD46 and CD59 or does not have detectable expression of CD46 and CD59. In some embodiments, the fold increase in expression compared to a cell lacking the modifications is greater than 200-fold.

In some embodiments, the modified pluripotent stem cells (comprising one or more modifications that increase expression of CD46 and CD59) comprises between 2-fold and 200-fold, between 2-fold and 100-fold, between 2-fold and 50-fold, or between 2-fold and 20-fold increased expression of CD46 and CD59 compared to a cell that does not have the modifications (e.g., compared to endogenous expression of CD46 and CD59). In some embodiments, the modified pluripotent stem cell (comprising one or more modifications that increase expression of CD46 and CD59) comprises between 5-fold and 200-fold, between 5-fold and 100-fold, between 5-fold and 50-fold, or between 5-fold and 20-fold increased expression of CD46 and CD59 compared to a cell that does not have the modifications (e.g., compared to endogenous expression of CD46 and CD59).

In some embodiments, the modified pluripotent stem cells (comprising one or more modifications that increase expression of CD46 and CD59) comprises increased expression of CD46 and CD59 relative to a cell that does not comprise the modifications (e.g., relative to endogenous expression of CD46 and CD59). In some embodiments, the modified pluripotent stem cell comprises at least at or about 2-fold, at least at or about 4-fold, at least at or about 6-fold, at least at or about 10-fold, at least at or about and 15-fold, at least at or about 20-fold, at least at or about 30-fold, at least at or about 50-fold, at least at or about 60-fold, at least at or about 70-fold, at least at or about 80-fold, at least at or about 100-fold, or any value between any of the foregoing values, increased expression of CD46 and CD59 compared to a cell that does not have the modifications (e.g., compared to endogenous expression of CD46 and CD59).

In some embodiments, the modified pluripotent stem cell (comprising one or more modifications that increase expression of CD46 and CD59) comprises increased expression of CD46 and CD59 relative to a cell that does not comprise the modifications (e.g., relative to endogenous expression of CD46 and CD59). In some embodiments, the modified cell comprises at or about 2-fold, at or about 4-fold, at or about 6-fold, at or about 10-fold, at or about and 15-fold, at or about 20-fold, at or about 30-fold, at or about 50-fold, at or about 60-fold, at or about 70-fold, at or about 80-fold, at or about 100-fold, or any value between any of the foregoing values, increased expression of CD46 and CD59 compared to a cell that does not have the modifications (e.g., compared to endogenous expression of CD46 and CD59).

In some embodiments, the cell comprises one or more transgenes encoding the CD46 and CD59. In some embodiments, the transgenes are monocistronic or multicistronic vectors, as described below. In some embodiments, the CD46 and CD59 are comprised by the same multicistronic vector, optionally in combination with one or more tolerogenic factors such as CD47. In some embodiments, the CD46 and CD59 are comprised by different transgenes, optionally in combination with one or more tolerogenic factors such as CD47.

In some embodiments, the modified pluripotent stem cell contains an overexpressed polynucleotide that encodes CD46, such as any described above, an overexpressed polynucleotide that encodes CD59, such as any described above, and an overexpressed polynucleotide that encodes CD55, such as any described above.

In some embodiments, the modified pluripotent stem cell contains an exogenous polynucleotide that encodes CD46, such as any described above, an exogenous polynucleotide that encodes CD59, such as any described above, and an exogenous polynucleotide that encodes CD55, such as any described above.

In some embodiments, the modified pluripotent stem cell (comprising one or more modifications that increase expression of CD46, CD59, and CD55) comprises increased expression of CD46, CD59, and CD55 relative to a cell that does not comprise the modifications (e.g., relative to endogenous expression of CD46, CD59, and CD55). In some embodiments, the modified cell comprises between 1.5-fold and 2-fold, between 2-fold and 3-fold, between 3-fold and 4-fold, between 4-fold and 5-fold, between 5-fold and 10-fold, between 10-fold and 15-fold, between 15-fold and 20-fold, between 20-fold and 40-fold, between 40-fold and 60-fold, between 60-fold and 80-fold, between 80-fold and 100-fold, or between 100-fold and 200-fold increased expression of CD46, CD59, and CD55 compared to a cell that does not have the modifications (e.g., compared to endogenous expression of CD46, CD59, and CD55). In some embodiments, the cell without the modification(s) does not have endogenous expression of CD46, CD59, and CD55or does not have detectable expression of CD46, CD59, and CD55. In some embodiments, the fold increase in expression compared to a cell lacking the modifications is greater than 200-fold.

In some embodiments, the modified pluripotent stem cell (comprising one or more modifications that increase expression of CD46, CD59, and CD55) comprises between 2-fold and 200-fold, between 2-fold and 100-fold, between 2-fold and 50-fold, or between 2-fold and 20-fold increased expression of CD46, CD59, and CD55 compared to a cell that does not have the modifications (e.g., compared to endogenous expression of CD46, CD59, and CD55). In some embodiments, the modified pluripotent stem cell (comprising one or more modifications that increase expression of CD46, CD59, and CD55) comprises between 5-fold and 200-fold, between 5-fold and 100-fold, between 5-fold and 50-fold, or between 5-fold and 20-fold increased expression of CD46, CD59, and CD55 compared to a cell that does not have the modifications (e.g., compared to endogenous expression of CD46, CD59, and CD55).

In some embodiments, the modified pluripotent stem cell (comprising one or more modifications that increase expression of CD46, CD59, and CD55) comprises increased expression of CD46, CD59, and CD55 relative to a cell that does not comprise the modifications (e.g., relative to endogenous expression of CD46 and CD59). In some embodiments, the modified cell comprises at least at or about 2-fold, at least at or about 4-fold, at least at or about 6-fold, at least at or about 10-fold, at least at or about and 15-fold, at least at or about 20-fold, at least at or about 30-fold, at least at or about 50-fold, at least at or about 60-fold, at least at or about 70-fold, at least at or about 80-fold, at least at or about 100-fold, or any value between any of the foregoing values, increased expression of CD46, CD59, and CD55 compared to a cell that does not have the modifications (e.g., compared to endogenous expression of CD46, CD59, and CD55).

In some embodiments, the modified pluripotent stem cell (comprising one or more modifications that increase expression of CD46, CD59, and CD55) comprises increased expression of CD46, CD59, and CD55 relative to a cell that does not comprise the modifications (e.g., relative to endogenous expression of CD46, CD59, and CD55). In some embodiments, the modified cell comprises at or about 2-fold, at or about 4-fold, at or about 6-fold, at or about 10-fold, at or about and 15-fold, at or about 20-fold, at or about 30-fold, at or about 50-fold, at or about 60-fold, at or about 70-fold, at or about 80-fold, at or about 100-fold, or any value between any of the foregoing values, increased expression of CD46, CD59, and CD55compared to a cell that does not have the modifications (e.g., compared to endogenous expression of CD46, CD59, and CD55).

In some embodiments, the cell comprises one or more transgenes encoding the CD46, CD59, and CD55. In some embodiments, the transgenes are monocistronic or multicistronic vectors, as described below. In some embodiments, the CD46, CD59, and CD55 are comprised by the same multicistronic vector, optionally in combination with one or more tolerogenic factors such as CD47. In some embodiments, the CD46, CD59, and CD55 are comprised by different transgenes, optionally in combination with one or more tolerogenic factors such as CD47.

e. Methods of Increasing Expression of (e.g. Overexpressing) a Polynucleotide

In some embodiments, increased expression of a polynucleotide may be carried out by any of a variety of techniques. For instance, methods for modulating expression of genes and factors (proteins) include genome editing technologies, and RNA or protein expression technologies and the like. For all of these technologies, well known recombinant techniques are used, to generate recombinant nucleic acids as outlined herein. In some embodiments, the cell that is modified with the one or more modification for overexpression or increased expression of a polynucleotide is any source cell as described herein.

### 9) DNA-Binding Fusion Proteins

In some embodiments, expression of a target gene (e.g., CD47, or another tolerogenic factor) is increased by expression of fusion protein or a protein complex containing (1) a site-specific binding domain specific for the endogenous CD47, or other gene and (2) a transcriptional activator.

In some embodiments, the regulatory factor is comprised of a site-specific DNA-binding nucleic acid molecule, such as a guide RNA (gRNA). In some embodiments, the method is achieved by site specific DNA-binding targeted proteins, such as zinc finger proteins (ZFP) or fusion proteins containing ZFP, which are also known as zinc finger nucleases (ZFNs).

In some embodiments, the regulatory factor comprises a site-specific binding domain, such as using a DNA binding protein or DNA-binding nucleic acid, which specifically binds to or hybridizes to the gene at a targeted region. In some embodiments, the provided polynucleotides or polypeptides are coupled to or complexed with a site-specific nuclease, such as a modified nuclease. For example, in some embodiments, the administration is effected using a fusion comprising a DNA-targeting protein of a modified nuclease, such as a meganuclease or an RNA-guided nuclease such as a clustered regularly interspersed short palindromic nucleic acid (CRISPR)-Cas system, such as CRISPR-Cas9 system. In some embodiments, the nuclease is modified to lack nuclease activity. In some embodiments, the modified nuclease is a catalytically dead dCas9.

In some embodiments, the site specific binding domain may be derived from a nuclease. For example, the recognition sequences of homing endonucleases and meganucleases such as I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII. See also U.S. Pat. Nos. 5,420,032; 6,833,252; Belfort et al., (1997) Nucleic Acids Res. 25:3379-3388; Dujon et al., (1989) Gene 82:115-118; Perler et al, (1994) Nucleic Acids Res. 22, 1125-1127; Jasin (1996) Trends Genet. 12:224-228; Gimble et al., (1996) J. Mol. Biol. 263:163-180; Argast et al, (1998) J. Mol. Biol. 280:345-353 and the New England Biolabs catalogue. In addition, the DNA-binding specificity of homing endonucleases and meganucleases can be modified to bind non-natural target sites. See, for example, Chevalier et al, (2002) Molec. Cell 10:895-905; Epinat et al, (2003) Nucleic Acids Res. 31:2952-2962; Ashworth et al, (2006) Nature 441:656-659; Paques et al, (2007) Current Gene Therapy 7:49-66; U.S. Patent Publication No. 2007/0117128.

Zinc finger, TALE, and CRISPR system binding domains can be “engineered” to bind to a predetermined nucleotide sequence, for example via engineering (altering one or more amino acids) of the recognition helix region of a naturally occurring zinc finger or TALE protein. Engineered DNA binding proteins (zinc fingers or TALEs) are proteins that are non-naturally occurring. Rational criteria for design include application of substitution rules and computerized algorithms for processing information in a database storing information of existing ZFP and/or TALE designs and binding data. See, for example, U.S. Pat. Nos. 6,140,081; 6,453,242; and 6,534,261; see also WO 98/53058; WO 98/53059; WO 98/53060; WO 02/016536 and WO 03/016496 and U.S. Publication No. 20110301073.

In some embodiments, the site-specific binding domain comprises one or more zinc-finger proteins (ZFPs) or domains thereof that bind to DNA in a sequence-specific manner. A ZFP or domain thereof is a protein or domain within a larger protein that binds DNA in a sequence-specific manner through one or more zinc fingers, regions of amino acid sequence within the binding domain whose structure is stabilized through coordination of a zinc ion.

Among the ZFPs are artificial ZFP domains targeting specific DNA sequences, typically 9-18 nucleotides long, generated by assembly of individual fingers. ZFPs include those in which a single finger domain is approximately 30 amino acids in length and contains an alpha helix containing two invariant histidine residues coordinated through zinc with two cysteines of a single beta turn, and having two, three, four, five, or six fingers. Generally, sequence-specificity of a ZFP may be altered by making amino acid substitutions at the four helix positions (−1, 2, 3 and 6) on a zinc finger recognition helix. Thus, in some embodiments, the ZFP or ZFP-containing molecule is non-naturally occurring, e.g., is engineered to bind to a target site of choice. See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; Choo et al. (2000) Curr. Opin. Struct. Biol. 10:411-416; U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; 2005/0267061, all incorporated herein by reference in their entireties.

Many gene-specific engineered zinc fingers are available commercially. For example, Sangamo Biosciences (Richmond, CA, USA) has developed a platform (CompoZr) for zinc-finger construction in partnership with Sigma-Aldrich (St. Louis, MO, USA), allowing investigators to bypass zinc-finger construction and validation altogether, and provides specifically targeted zinc fingers for thousands of proteins (Gaj et al., Trends in Biotechnology, 2013, 31 (7), 397-405). In some embodiments, commercially available zinc fingers are used or are custom designed.

In some embodiments, the site-specific binding domain comprises a naturally occurring or engineered (non-naturally occurring) transcription activator-like protein (TAL) DNA binding domain, such as in a transcription activator-like protein effector (TALE) protein, See, e.g., U.S. Patent Publication No. 20110301073, incorporated by reference in its entirety herein.

In some embodiments, the site-specific binding domain is derived from the CRISPR/Cas system. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system, or a “targeting sequence”), and/or other sequences and transcripts from a CRISPR locus.

In general, a guide sequence includes a targeting domain comprising a polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. In some examples, the targeting domain (e.g., targeting sequence) of the gRNA is complementary, e.g., at least 80, 85, 90, 95, 98 or 99% complementary, e.g., fully complementary, to the target sequence on the target nucleic acid.

In some embodiments, the gRNA may be any as described herein. In particular embodiments, the gRNA has a targeting sequence that is complementary to a target site of CD47, such as set forth in any one of SEQ ID NOS: 200784-231885 (Table 29, Appendix 22 of WO2016183041); HLA-E, such as set forth in any one of SEQ ID NOS: 189859-193183 (Table 19, Appendix 12 of WO2016183041); HLA-F, such as set forth in any one of SEQ ID NOS: 688808-699754 (Table 45, Appendix 38 of WO2016183041); HLA-G, such as set forth in any one of SEQ ID NOS: 188372-189858 (Table 18, Appendix 11 of WO2016183041); or PD-L1, such as set forth in any one of SEQ ID NOS: 193184-200783 (Table 21, Appendix 14 of WO2016183041).

In some embodiments, the target site is upstream of a transcription initiation site of the target gene. In some embodiments, the target site is adjacent to a transcription initiation site of the gene. In some embodiments, the target site is adjacent to an RNA polymerase pause site downstream of a transcription initiation site of the gene.

In some embodiments, the targeting domain is configured to target the promoter region of the target gene to promote transcription initiation, binding of one or more transcription enhancers or activators, and/or RNA polymerase. One or more gRNA can be used to target the promoter region of the gene. In some embodiments, one or more regions of the gene can be targeted. In certain aspects, the target sites are within 600 base pairs on either side of a transcription start site (TSS) of the gene.

It is within the level of a skilled artisan to design or identify a gRNA sequence that is or comprises a sequence targeting a gene (i.e., gRNA targeting sequence), including the exon sequence and sequences of regulatory regions, including promoters and activators. A genome-wide gRNA database for CRISPR genome editing is publicly available, which contains exemplary single guide RNA (sgRNA) target sequences in constitutive exons of genes in the human genome or mouse genome (see e.g., genescript.com/gRNA-database.html; see also, Sanjana et al. (2014) Nat. Methods, 11:783-4; www.e-crisp.org/E-CRISP/; crispr.mit.edu/). In some embodiments, the gRNA sequence is or comprises a targeting sequence with minimal off-target binding to a non-target gene.

In some embodiments, the regulatory factor further comprises a functional domain, e.g., a transcriptional activator.

In some embodiments, the transcriptional activator is or contains one or more regulatory elements, such as one or more transcriptional control elements of a target gene, whereby a site-specific domain as provided above is recognized to drive expression of such gene. In some embodiments, the transcriptional activator drives expression of the target gene. In some cases, the transcriptional activator, can be or contain all or a portion of a heterologous transactivation domain. For example, in some embodiments, the transcriptional activator is selected from Herpes simplex-derived transactivation domain, Dnmt3a methyltransferase domain, p65, VP16, and VP64.

In some embodiments, the regulatory factor is a zinc finger transcription factor (ZF-TF). In some embodiments, the regulatory factor is VP64-p65-Rta (VPR).

In certain embodiments, the regulatory factor further comprises a transcriptional regulatory domain. Common domains include, e.g., transcription factor domains (activators, repressors, co-activators, co-repressors), silencers, oncogenes (e.g., myc, jun, fos, myb, max, mad, rel, ets, bcl, myb, mos family members etc.); DNA repair enzymes and their associated factors and modifiers; DNA rearrangement enzymes and their associated factors and modifiers; chromatin associated proteins and their modifiers (e.g. kinases, acetylases and deacetylases); and DNA modifying enzymes (e.g., methyltransferases such as members of the DNMT family (e.g., DNMT1, DNMT3A, DNMT3B, DNMT3L, etc., topoisomerases, helicases, ligases, kinases, phosphatases, polymerases, endonucleases) and their associated factors and modifiers. See, e.g., U.S. Publication No. 2013/0253040, incorporated by reference in its entirety herein.

Suitable domains for achieving activation include the HSV VP 16 activation domain (see, e.g., Hagmann et al, J. Virol. 71, 5952-5962 (1 97)) nuclear hormone receptors (see, e.g., Torchia et al., Curr. Opin. Cell. Biol. 10:373-383 (1998)); the p65 subunit of nuclear factor kappa B (Bitko & Bank, J. Virol. 72:5610-5618 (1998) and Doyle & Hunt, Neuroreport 8:2937-2942 (1997)); Liu et al., Cancer Gene Ther. 5:3-28 (1998)), or artificial chimeric functional domains such as VP64 (Beerli et al., (1998) Proc. Natl. Acad. Sci. USA 95:14623-33), and degron (Molinari et al., (1999) EMBO J. 18, 6439-6447). Additional exemplary activation domains include, Oct 1, Oct-2A, Spl, AP-2, and CTF1 (Seipel et al, EMBOJ. 11, 4961-4968 (1992) as well as p300, CBP, PCAF, SRC1 PvALF, AtHD2A and ERF-2. See, for example, Robyr et al, (2000) Mol. Endocrinol. 14:329-347; Collingwood et al, (1999) J. Mol. Endocrinol 23:255-275; Leo et al, (2000) Gene 245:1-11; Manteuffel-Cymborowska (1999) Acta Biochim. Pol. 46:77-89; McKenna et al, (1999) J. Steroid Biochem. Mol. Biol. 69:3-12; Malik et al, (2000) Trends Biochem. Sci. 25:277-283; and Lemon et al, (1999) Curr. Opin. Genet. Dev. 9:499-504. Additional exemplary activation domains include, but are not limited to, OsGAI, HALF-1, Cl, AP1, ARF-5, -6, -1, and -8, CPRF1, CPRF4, MYC-RP/GP, and TRAB1, See, for example, Ogawa et al, (2000) Gene 245:21-29; Okanami et al, (1996) Genes Cells 1:87-99; Goff et al, (1991) Genes Dev. 5:298-309; Cho et al, (1999) Plant Mol Biol 40:419-429; Ulmason et al, (1999) Proc. Natl. Acad. Sci. USA 96:5844-5849; Sprenger-Haussels et al, (2000) Plant J. 22:1-8; Gong et al, (1999) Plant Mol. Biol. 41:33-44; and Hobo et al., (1999) Proc. Natl. Acad. Sci. USA 96:15,348-15,353.

Exemplary repression domains that can be used to make genetic repressors include, but are not limited to, KRAB A/B, KOX, TGF-beta-inducible early gene (TIEG), v-erbA, SID, MBD2, MBD3, members of the DNMT family (e.g., DNMT1, DNMT3A, DNMT3B, DNMT3L, etc.), Rb, and MeCP2. See, for example, Bird et al, (1999) Cell 99:451-454; Tyler et al, (1999) Cell 99:443-446; Knoepfler et al, (1999) Cell 99:447-450; and Robertson et al, (2000) Nature Genet. 25:338-342. Additional exemplary repression domains include, but are not limited to, ROM2 and AtHD2A. See, for example, Chem et al, (1996) Plant Cell 8:305-321; and Wu et al, (2000) Plant J. 22:19-27.

In some instances, the domain is involved in epigenetic regulation of a chromosome. In some embodiments, the domain is a histone acetyltransferase (HAT), e.g., type-A, nuclear localized such as MYST family members MOZ, Ybf2/Sas3, MOF, and Tip60, GNAT family members Gcn5 or pCAF, the p300 family members CBP, p300 or Rtt109 (Bemdsen and Denu (2008) Curr Opin Struct Biol 18 (6): 682-689). In other instances, the domain is a histone deacetylase (HD AC) such as the class I (HDAC-1, 2, 3, and 8), class II (HDAC IIA (HDAC-4, 5, 7 and 9), HD AC IIB (HDAC 6 and 10)), class IV (HDAC-11), class III (also known as sirtuins (SIRTs); SIRT1-7) (see Mottamal et al., (2015) Molecules 20 (3): 3898-3941). Another domain that is used in some embodiments is a histone phosphorylase or kinase, where examples include MSK1, MSK2, ATR, ATM, DNA-PK, Bubl, VprBP, IKK-a, PKCpi, Dik/Zip, JAK2, PKC5, WSTF and CK2. In some embodiments, a methylation domain is used and may be chosen from groups such as Ezh2, PRMT1/6, PRMT5/7, PRMT 2/6, CARM1, set7/9, MLL, ALL-1, Suv 39h, G9a, SETDB1, Ezh2, Set2, Dotl, PRMT 1/6, PRMT 5/7, PR-Set7 and Suv4-20h, Domains involved in sumoylation and biotinylation (Lys9, 13, 4, 18 and 12) may also be used in some embodiments (review see Kousarides (2007) Cell 128:693-705).

Fusion molecules are constructed by methods of cloning and biochemical conjugation that are well known to those of skill in the art. Fusion molecules comprise a DNA-binding domain and a functional domain (e.g., a transcriptional activation or repression domain). Fusion molecules also optionally comprise nuclear localization signals (such as, for example, that from the SV40 medium T-antigen) and epitope tags (such as, for example, FLAG and hemagglutinin). Fusion proteins (and nucleic acids encoding them) are designed such that the translational reading frame is preserved among the components of the fusion.

Fusions between a polypeptide component of a functional domain (or a functional fragment thereof) on the one hand, and a non-protein DNA-binding domain (e.g., antibiotic, intercalator, minor groove binder, nucleic acid) on the other, are constructed by methods of biochemical conjugation known to those of skill in the art. See, for example, the Pierce Chemical Company (Rockford, IL) Catalogue. Methods and compositions for making fusions between a minor groove binder and a polypeptide have been described. Mapp et al, (2000) Proc. Natl. Acad. Sci. USA 97:3930-3935. Likewise, CRISPR/Cas TFs and nucleases comprising a sgRNA nucleic acid component in association with a polypeptide component function domain are also known to those of skill in the art and detailed herein.

### 10) Exogenous Polypeptide

In some embodiments, increased expression (i.e., overexpression) of the polynucleotide is mediated by introducing into the cell an exogenous polynucleotide encoding the polynucleotide to be overexpressed. In some embodiments, the exogenous polynucleotide is a recombinant nucleic acid. Well-known recombinant techniques can be used to generate recombinant nucleic acids as outlined herein. In some embodiments, an exogenous polynucleotide encoding an exogenous polypeptide herein comprises a codon-optimized nucleic acid sequence.

In certain embodiments, the recombinant nucleic acids encoding an exogenous polypeptide, such as a tolerogenic factor or a chimeric antigen receptor, may be operably linked to one or more regulatory nucleotide sequences in an expression construct. Regulatory nucleotide sequences will generally be appropriate for the host cell and recipient subject to be treated. Numerous types of appropriate expression vectors and suitable regulatory sequences are known in the art for a variety of host cells. Typically, the one or more regulatory nucleotide sequences may include, but are not limited to, promoter sequences, leader or signal sequences, ribosomal binding sites, transcriptional start and termination sequences, translational start and termination sequences, and enhancer or activator sequences. Constitutive or inducible promoters as known in the art are also contemplated. The promoters may be either naturally occurring promoters, or hybrid promoters that combine elements of more than one promoter. An expression construct may be present in a cell on an episome, such as a plasmid, or the expression construct may be inserted in a chromosome. In a specific embodiment, the expression vector includes a selectable marker gene to allow the selection of transformed host cells. Certain embodiments include an expression vector comprising a nucleotide sequence encoding a variant polypeptide operably linked to at least one regulatory sequence. Regulatory sequence for use herein include promoters, enhancers, and other expression control elements. In certain embodiments, an expression vector is designed for the choice of the host cell to be transformed, the particular variant polypeptide desired to be expressed, the vector's copy number, the ability to control that copy number, and/or the expression of any other protein encoded by the vector, such as antibiotic markers.

In some embodiments, the exogenous polynucleotide is operably linked to a promoter for expression of the exogenous polynucleotide in the modified cell. Examples of suitable mammalian promoters include, for example, promoters from the following genes: elongation factor 1 alpha (EF1α) promoter, ubiquitin/S27a promoter of the hamster (WO 97/15664), Simian vacuolating virus 40 (SV40) early promoter, adenovirus major late promoter, mouse metallothionein-I promoter, the long terminal repeat region of Rous Sarcoma Virus (RSV), mouse mammary tumor virus promoter (MMTV), Moloney murine leukemia virus Long Terminal repeat region, and the early promoter of human Cytomegalovirus (CMV). Examples of other heterologous mammalian promoters are the actin, immunoglobulin or heat shock promoter(s). In additional embodiments, promoters for use in mammalian host cells can be obtained from the genomes of viruses such as polyoma virus, fowlpox virus (UK 2,211,504 published 5 Jul. 1989), bovine papilloma virus, avian sarcoma virus, cytomegalovirus, a retrovirus, hepatitis-B virus and Simian Virus 40 (SV40). In further embodiments, heterologous mammalian promoters are used. Examples include the actin promoter, an immunoglobulin promoter, and heat-shock promoters. The early and late promoters of SV40 are conveniently obtained as an SV40 restriction fragment which also contains the SV40 viral origin of replication (Fiers et al, Nature 273:113-120 (1978)). The immediate early promoter of the human cytomegalovirus is conveniently obtained as a HindIII restriction enzyme fragment (Greenaway et al, Gene 18:355-360 (1982)). The foregoing references are incorporated by reference in their entirety.

In some embodiments, the expression vector is a bicistronic or multicistronic expression vector. Bicistronic or multicistronic expression vectors may include (1) multiple promoters fused to each of the open reading frames; (2) insertion of splicing signals between genes; (3) fusion of genes whose expressions are driven by a single promoter; and/or (4) insertion of proteolytic cleavage sites between genes (self-cleavage peptide) or insertion of internal ribosomal entry sites (IRESs) between genes.

In some embodiments, an expression vector or construct herein is a multicistronic construct. The terms “multicistronic construct” and “multicistronic vector” are used interchangeably herein and refer to a recombinant DNA construct that is to be transcribed into a single mRNA molecule, wherein the single mRNA molecule encodes two or more genes (e.g., two or more transgenes). The multi-cistronic construct is referred to as bicistronic construct if it encodes two genes, and tricistronic construct if it encodes three genes, and quadrocistronic construct if it encodes four genes, and so on.

In some embodiments, two or more exogenous polynucleotides comprised by a vector or construct (e.g., a transgene) are each separated by a multicistronic separation element. In some embodiments, the multicistronic separation element is an IRES or a sequence encoding a cleavable peptide or ribosomal skip element. In some embodiments, the multicistronic separation element is an IRES, such as an encephalomyocarditis (EMCV) virus IRES. In some embodiments, the multicistronic separation element is a cleavable peptide such as a 2A peptide. Exemplary 2A peptides include a P2A peptide, a T2A peptide, an E2A peptide, and an F2Apeptide. In some embodiments, the cleavable peptide is a T2A. In some embodiments, the two or more exogenous polynucleotides (e.g. the first exogenous polynucleotide and second exogenous polynucleotide) are operably linked to a promoter. In some embodiments, the first exogenous polynucleotide and the second exogenous polynucleotide are each operably linked to a promoter. In some embodiments, the promoter is the same promoter. In some embodiments, the promoter is an EF1 promoter.

In some cases, an exogenous polynucleotide encoding an exogenous polypeptide (e.g., an exogenous polynucleotide encoding a tolerogenic factor or complement inhibitor described herein) encodes a cleavable peptide or ribosomal skip element, such as T2A at the N-terminus or C-terminus of an exogenous polypeptide encoded by a multicistronic vector. In some embodiments, inclusion of the cleavable peptide or ribosomal skip element allows for expression of two or more polypeptides from a single translation initiation site. In some embodiments, the cleavable peptide is a T2A. In some embodiments, the T2A is or comprises the amino acid sequence set forth by SEQ ID NO: 11. In some embodiments, the T2A is or comprises the amino acid sequence set forth by SEQ ID NO: 12. In some embodiments, the T2A is or comprises the amino acid sequence set forth by SEQ ID NO: 17. In some embodiments, the T2A is or comprises the amino acid sequence set forth by SEQ ID NO: 18.

In some embodiments, the vector or construct includes a single promoter that drives the expression of one or more transcription units of an exogenous polynucleotide. In some embodiments, such vectors or constructs can be multicistronic (bicistronic or tricistronic, see e.g., U.S. Pat. No. 6,060,273). For example, in some embodiments, transcription units can be engineered as a bicistronic unit containing an IRES (internal ribosome entry site), which allows coexpression of gene products (e.g., one or more tolerogenic factors such as CD47 and/or one or more complement inhibitor such as CD46, CD59, and CD55) from an RNA transcribed from a single promoter. In some embodiments, the vectors or constructs provided herein are bicistronic, allowing the vector or construct to express two separate polypeptides. In some cases, the two separate polypeptides encoded by the vector or construct are tolerogenic factors (e.g., two factors selected from CD47, DUX4, CD24, CD27, CD200, HLA-C, HLA-E, HLA-E heavy chain, HLA-G, PD-L1, IDO1, CTLA4-Ig, Cl-Inhibitor, IL-10, IL-35, IL-39, FasL, CCL21, CCL22, Mfge8, and Serpinb9). In some embodiments, the one or more tolerogenic factors are selected from the group consisting of CD16, CD24, CD35, CD39, CD46, CD47, CD52, CD55, CD59, CD64, CD200, CCL22, CTLA4-Ig, C1 inhibitor, FASL, IDO1, HLA-C, HLA-E, HLA-E heavy chain, HLA-G, IL-10, IL-35, PD-L1, SERPINB9, CCL21, MFGE8, DUX4, B2M-HLA-E, CD27, IL-39, CD16 Fc Receptor, IL15-RF, H2-M3 (HLA-G), A20/TNFAIP3, CR1, HLA-F, and MANF. In some cases, the two separate polypeptides encoded by the vector or construct are CD46 and CD59. In some embodiments, the two separate polypeptides encoded by the vector or construct are a tolerogenic factor (e.g., CD47) and a complement inhibitor selected from CD46, CD59, and CD55. In some embodiments, the vectors or constructs provided herein are tricistronic, allowing the vector or construct to express three separate polypeptides. In some cases, the three nucleic acid sequences of the tricistronic vector or construct are a tolerogenic factor such as CD47, CD46, and CD59. In some cases, the three nucleic acid sequences of the tricistronic vector or construct are CD46, CD59, and CD55. In some cases, the three nucleic acid sequences of the tricistronic vector or construct are three tolerogenic factors selected from CD47, DUX4, CD24, CD27, CD200, HLA-C, HLA-E, HLA-E heavy chain, HLA-G, PD-L1, IDO1, CTLA4-Ig, Cl-Inhibitor, IL-10, IL-35, IL-39, FasL, CCL21, CCL22, Mfge8, and Serpinb9. In some embodiments, the vectors or constructs provided herein are quadrocistronic, allowing the vector or construct to express four separate polypeptides. In some cases, the four separate polypeptides of the quadrocistronic vector or construct are CD47, CD46, CD59, and CD55. In some cases, the four separate polypeptides of the quadrocistronic vector or construct are four tolerogenic factors selected from CD47, DUX4, CD24, CD27, CD200, HLA-C, HLA-E, HLA-E heavy chain, HLA-G, PD-L1, IDO1, CTLA4-Ig, Cl-Inhibitor, IL-10, IL-35, IL-39, FasL, CCL21, CCL22, Mfge8, and Serpinb9.

In some embodiments, the cell comprises one or more vectors or constructs, wherein each vector or construct is a monocistronic or a multicistronic construct as described above, and the monocistronic or multicistronic constructs encode one or more tolerogenic factors and/or complement inhibitors, in any combination or order.

In some embodiments, a single promoter directs expression of an RNA that contains, in a single open reading frame (ORF), two, three, or four genes (e.g., encoding a tolerogenic factor (e.g., CD47) and/or one or more complement inhibitors selected from CD46, CD59, and CD55) separated from one another by sequences encoding a self-cleavage peptide (e.g., 2A sequences) or a protease recognition site (e.g., furin). The ORF thus encodes a single polypeptide, which, either during (in the case of 2A) or after translation, is processed into the individual proteins. In some cases, the peptide, such as T2A, can cause the ribosome to skip (ribosome skipping) synthesis of a peptide bond at the C-terminus of a 2A element, leading to separation between the end of the 2A sequence and the next peptide downstream (see, for example, de Felipe. Genetic Vaccines and Ther. 2:13 (2004) and deFelipe et al. Traffic 5:616-626 (2004)). Many 2A elements are known in the art. Examples of 2A sequences that can be used in the methods and nucleic acids disclosed herein include, without limitation, 2A sequences from the foot-and-mouth disease virus (F2A, e.g., SEQ ID NO: 16), equine rhinitis A virus (E2A, e.g., SEQ ID NO: 15), thosea asigna virus (T2A, e.g., SEQ ID NO: 11, 12, 17, or 18), and porcine teschovirus-1 (P2A, e.g., SEQ ID NO: 13 or 14) as described in U.S. Patent Publication No. 20070116690.

In cases where the vector or construct (e.g., transgene) contains more than one nucleic acid sequence encoding a protein, e.g., a first exogenous polynucleotide encoding CD46 and a second exogenous polynucleotide encoding CD59, or a first exogenous polynucleotide encoding CD47, a second exogenous polynucleotide encoding CD56, and a third exogenous polynucleotide encoding CD59, the vector or construct (e.g., transgene) may further include a nucleic acid sequence encoding a peptide between the first and second exogenous polynucleotide sequences. In some cases, the nucleic acid sequence positioned between the first and second exogenous polynucleotides encodes a peptide that separates the translation products of the first and second exogenous polynucleotides during or after translation. In some embodiments, the peptide contains a self-cleaving peptide or a peptide that causes ribosome skipping (a ribosomal skip element), such as a T2A peptide. In some embodiments, inclusion of the cleavable peptide or ribosomal skip element allows for expression of two or more polypeptides from a single translation initiation site. In some embodiments, the peptide is a self-cleaving peptide that is a T2A peptide. In some embodiments, the T2A is or comprises the amino acid sequence set forth by SEQ ID NO: 11. In some embodiments, the T2A is or comprises the amino acid sequence set forth by SEQ ID NO: 12. In some embodiments, the T2A is or comprises the amino acid sequence set forth by SEQ ID NO: 17. In some embodiments, the T2A is or comprises the amino acid sequence set forth by SEQ ID NO: 18.

The process of introducing the polynucleotides described herein into cells can be achieved by any suitable technique. Suitable techniques include calcium phosphate or lipid-mediated transfection, electroporation, fusogens, and transduction or infection using a viral vector. In some embodiments, the polynucleotides are introduced into a cell via viral transduction (e.g., lentiviral transduction) or otherwise delivered on a viral vector (e.g., fusogen-mediated delivery). Suitable techniques include calcium phosphate or lipid-mediated transfection, electroporation, transposase-mediated delivery, and transduction or infection using a viral vector. In some embodiments, the polynucleotides are introduced into a cell via viral transduction (e.g., lentiviral transduction) or otherwise delivered on a viral vector (e.g., fusogen-mediated delivery). In some embodiments, vectors that package a polynucleotide encoding an exogenous polynucleotide may be used to deliver the packaged polynucleotides to a cell or population of cells. These vectors may be of any kind, including DNA vectors, RNA vectors, plasmids, viral vectors and particles. In some embodiments, lipid nanoparticles can be used to deliver an exogenous polynucleotide to a cell. In some embodiments, viral vectors can be used to deliver an exogenous polynucleotide to a cell. Viral vector technology is well known and described in Sambrook et al. (2001, Molecular Cloning: A Laboratory Manual, Cold Spring Harbor Laboratory, New York). Viruses, which are useful as vectors include, but are not limited to lentiviral vectors, adenoviral vectors, adeno-associated viral (AAV) vectors, herpes simplex viral vectors, retroviral vectors, oncolytic viruses, and the like. In some embodiments, the introduction of the exogenous polynucleotide into the cell can be specific (targeted) or non-specific (e.g., non-targeted). In some embodiments, the introduction of the exogenous polynucleotide into the cell can result in integration or insertion into the genome in the cell. In other embodiments, the introduced exogenous polynucleotide may be non-integrating or episomal in the cell. A skilled artisan is familiar with methods of introducing nucleic acid transgenes into a cell, including any of the exemplary methods described herein, and can choose a suitable method.

### E) Non-Targeted Delivery

In some embodiments, an exogenous polynucleotide is introduced into a cell (e.g., source cell) by any of a variety of non-targeted methods. In some embodiments, the exogenous polynucleotide is inserted into a random genomic locus of a host cell. As known to a person skilled in the art, viral vectors, including, for example, retroviral vectors and lentiviral vectors are commonly used to deliver genetic material into host cells and randomly insert the foreign or exogenous gene into the host cell genome to facilitate stable expression and replication of the gene. In some embodiments, the non-targeted introduction of the exogenous polynucleotide into the cell is under conditions for stable expression of the exogenous polynucleotide in the cell. In some embodiments, methods for introducing a nucleic acid for stable expression in a cell involves any method that results in stable integration of the nucleic acid into the genome of the cell, such that it may be propagated if the cell it has integrated into divides.

In some embodiments, the viral vector is a lentiviral vector. Lentiviral vectors are particularly useful means for successful viral transduction as they permit stable expression of the gene contained within the delivered nucleic acid transcript. Lentiviral vectors express reverse transcriptase and integrase, two enzymes required for stable expression of the gene contained within the delivered nucleic acid transcript. Reverse transcriptase converts an RNA transcript into DNA, while integrase inserts and integrates the DNA into the genome of the target cell. Once the DNA has been integrated stably into the genome, it divides along with the host. The gene of interest contained within the integrated DNA may be expressed constitutively or it may be inducible. As part of the host cell genome, it may be subject to cellular regulation, including activation or repression, depending on a host of factors in the target cell.

Lentiviruses are subgroup of the Retroviridae family of viruses, named because reverse transcription of viral RNA genomes to DNA is required before integration into the host genome. As such, the most important features of lentiviral vehicles/particles are the integration of their genetic material into the genome of a target/host cell. Some examples of lentivirus include the Human Immunodeficiency Viruses: HIV-1 and HIV-2, the Simian Immunodeficiency Virus (SIV), feline immunodeficiency virus (FIV), bovine immunodeficiency virus (BIV), Jembrana Disease Virus (JDV), equine infectious anemia virus (EIAV), equine infectious anemia, virus, visna-maedi and caprine arthritis encephalitis virus (CAEV).

Typically, lentiviral particles making up the gene delivery vehicle are replication defective on their own (also referred to as “self-inactivating”). Lentiviruses are able to infect both dividing and non-dividing cells by virtue of the entry mechanism through the intact host nuclear envelope (Naldini L et al., Curr. Opin. Biotechnol, 1998, 9:457-463). Recombinant lentiviral vehicles/particles have been generated by multiply attenuating the HIV virulence genes, for example, the genes Env, Vif, Vpr, Vpu, Nef and Tat are deleted making the vector biologically safe. Correspondingly, lentiviral vehicles, for example, derived from HIV-1/HIV-2 can mediate the efficient delivery, integration and long-term expression of transgenes into non-dividing cells.

Lentiviral particles may be generated by co-expressing the virus packaging elements and the vector genome itself in a producer cell such as human HEK293T cells. These elements are usually provided in three (in second generation lentiviral systems) or four separate plasmids (in third generation lentiviral systems). The producer cells are co-transfected with plasmids that encode lentiviral components including the core (i.e., structural proteins) and enzymatic components of the virus, and the envelope protein(s) (referred to as the packaging systems), and a plasmid that encodes the genome including a foreign transgene, to be transferred to the target cell, the vehicle itself (also referred to as the transfer vector). In general, the plasmids or vectors are included in a producer cell line. The plasmids/vectors are introduced via transfection, transduction or infection into the producer cell line. Methods for transfection, transduction or infection are well known by those of skill in the art. As non-limiting example, the packaging and transfer constructs can be introduced into producer cell lines by calcium phosphate transfection, lipofection or electroporation, generally together with a dominant selectable marker, such as neomyocin (neo), dihydrofolate reductase (DHFR), glutamine synthetase or adenosine deaminase (ADA), followed by selection in the presence of the appropriate drug and isolation of clones.

The producer cell produces recombinant viral particles that contain the foreign gene, for example, the polynucleotides encoding the exogenous polynucleotide. The recombinant viral particles are recovered from the culture media and titrated by standard methods used by those of skill in the art. The recombinant lentiviral vehicles can be used to infect target cells, such source cells including any described herein.

Cells that can be used to produce high-titer lentiviral particles may include, but are not limited to, HEK293T cells, 293G cells, STAR cells (Relander et al., Mol Ther. 2005, 11:452-459), FreeStyle™ 293 Expression System (ThermoFisher, Waltham, MA), and other HEK293T-based producer cell lines (e.g., Stewart et al., Hum Gene Ther._2011, 2,2. (3): 357˜369; Lee et al, Biotechnol Bioeng, 2012, 10996): 1551-1560; Throm et al., Blood. 2009, 113 (21): 5104-5110).

Additional elements provided in lentiviral particles may comprise retroviral LTR (long-terminal repeat) at either 5′ or 3′ terminus, a retroviral export element, optionally a lentiviral reverse response element (RRE), a promoter or active portion thereof, and a locus control region (LCR) or active portion thereof. Other elements include central polypurine tract (cPPT) sequence to improve transduction efficiency in non-dividing cells, Woodchuck Hepatitis Virus (WHP) Posttranscriptional Regulatory Element (WPRE) which enhances the expression of the transgene, and increases titer.

Methods for generating recombinant lentiviral particles are known to a skilled artisan, for example, U.S. Pat. Nos. 8,846,385; 7,745,179; 7,629,153; 7,575,924; 7,179,903; and 6,808,905. Lentivirus vectors used may be selected from, but are not limited to pLVX, pLenti, pLenti6, pLJMI, FUGW, pWPXL, pWPI, pLenti CMV puro DEST, pLJMI-EGFP, pULTRA, pInducer2Q, pHIV-EGFP, pCW57.1, pTRPE, pELPS, pRRL, and pLionII, Any known lentiviral vehicles may also be used (See, U.S. Pat. Nos. 9,260,725: 9,068,199: 9,023,646: 8,900,858: 8,748,169; 8,709,799; 8,420,104; 8,329,462; 8,076,106; 6,013,516; and 5,994,136; International Patent Publication NO.: WO2012079000).

In some embodiments, the exogenous polynucleotide is introduced into the cell under conditions for transient expression of the cell, such as by methods that result in episomal delivery of an exogenous polynucleotide.

In some embodiments, polynucleotides encoding the exogenous polynucleotide may be packaged into recombinant adeno-associated viral (rAAV) vectors. Such vectors or viral particles may be designed to utilize any of the known serotype capsids or combinations of serotype capsids. The serotype capsids may include capsids from any identified AAV serotypes and variants thereof, for example, AAV1, AAV2, AAV2G9, AAV3, AAV4, AAV4-4, AAV5, AAV6, AAV7, AAV8, AAV9, AAV10, AAV11, AAV12 and AAVrh10. In some embodiments, the AAV serotype may be or have a sequence as described in United States Publication No. US20030138772; Pulicherla et al. Molecular Therapy, 2011, 19 (6): 1070-1078; U.S. Pat. Nos. 6,156,303; 7,198,951; U.S. Patent Publication Nos.: US2015/0159173 and US2014/0359799; and International Patent Publication NOs.: WO1998/011244, WO2005/033321 and WO2014/14422.

AAV vectors include not only single stranded vectors but self-complementary AAV vectors (scAAVs). scAAV vectors contain DNA which anneals together to form double stranded vector genome. By skipping second strand synthesis, scAAVs allow for rapid expression in the cell. The rAAV vectors may be manufactured by standard methods in the art such as by triple transfection, in sf9 insect cells or in suspension cell cultures of human cells such as HEK293 cells.

In some embodiments, non-viral based methods may be used. For instance, in some aspects, vectors comprising the polynucleotides may be transferred to cells by non-viral methods by physical methods such as needles, electroporation, sonoporation, hyrdoporation; chemical carriers such as inorganic particles (e.g., calcium phosphate, silica, gold) and/or chemical methods. In other aspects, synthetic or natural biodegradable agents may be used for delivery such as cationic lipids, lipid nano emulsions, nanoparticles, peptide-based vectors, or polymer-based vectors.

### F) Targeted Delivery

In some embodiments, the exogenous polynucleotide can be inserted into a specific genomic locus of the host cell. A number of gene editing methods can be used to insert an exogenous polynucleotide (e.g., a transgene) into a specific genomic locus of choice. The exogenous polynucleotide can be inserted into any suitable target genomic loci of the cell. In some embodiments, the exogenous polynucleotide is introduced into the cell by targeted integration into a target loci. In some embodiments, targeted integration can be achieved by gene editing using one or more nucleases and/or nickases and a donor template in a process involving homology-dependent or homology-independent recombination.

Gene editing is a type of genetic engineering in which a nucleotide sequence may be inserted, deleted, modified, or replaced in the genome of a living organism. A number of gene editing methods can be used to insert an exogenous polynucleotide into the specific genomic locus of choice, including for example homology-directed repair (HOR), homology-mediated end-joining (HMEJ), homology-independent targeted integration (HITI), obligate ligation-gated recombination (ObliGaRe), or precise integration into target chromosome (PITCh). In some embodiments, the gene editing technology can include systems involving nucleases, integrases, transposases, and/or recombinases. In some embodiments, the gene editing technology mediates single-strand breaks (SSB). In some embodiments, the gene editing technology mediates double-strand breaks (DSB), including in connection with non-homologous end-joining (NHEJ) or homology-directed repair (HDR). In some embodiments, the gene editing technology can include DNA-based editing or prime-editing. In some embodiments, the gene editing technology can include Programmable Addition via Site-specific Targeting Elements (PASTE). In some embodiments, the gene editing technology can include TnpB polypeptides. Many gene editing techniques generally utilize the innate mechanism for cells to repair double-strand breaks (DSBs) in DNA.

Eukaryotic cells repair DSBs by two primary repair pathways: non-homologous end-joining (NHEJ) and homology-directed repair (HDR). HDR typically occurs during late S phase or G2 phase, when a sister chromatid is available to serve as a repair template. NHEJ is more common and can occur during any phase of the cell cycle, but it is more error prone. In gene editing, NHEJ is generally used to produce insertion/deletion mutations (indels), which can produce targeted loss of function in a target gene by shifting the open reading frame (ORF) and producing alterations in the coding region or an associated regulatory region. HDR, on the other hand, is a preferred pathway for producing targeted knock-ins, knockouts, or insertions of specific mutations in the presence of a repair template with homologous sequences. Several methods are known to a skilled artisan to improve HDR efficiency, including, for example, chemical modulation (e.g., treating cells with inhibitors of key enzymes in the NHEJ pathway); timed delivery of the gene editing system at S and G2 phases of the cell cycle; cell cycle arrest at S and G2 phases; and introduction of repair templates with homology sequences. The methods provided herein may utilize HDR-mediated repair, NHEJ-mediated repair, or a combination thereof.

In some embodiments, the methods provided herein for HDR-mediated insertion utilize a site-directed nuclease, including, for example, zinc finger nucleases (ZFNs), transcription activator-like effector nucleases (TALENs), meganucleases, transposases, and clustered regularly interspaced short palindromic repeat (CRISPR)/Cas systems. In some embodiments, the nucleases create specific double-strand breaks (DSBs) at desired locations (e.g., target sites) in the genome, and harness the cell's endogenous mechanisms to repair the induced break. The nickases create specific single-strand breaks at desired locations in the genome. In one non-limiting example, two nickases can be used to create two single-strand breaks on opposite strands of a target DNA, thereby generating a blunt or a sticky end. Any suitable nuclease can be introduced into a cell to induce genome editing of a target DNA sequence including, but not limited to, CRISPR-associated protein (Cas) nucleases, zinc finger nucleases (ZFNs), transcription activator-like effector nucleases (TALENs), meganucleases, other endo- or exo-nucleases, variants thereof, fragments thereof, and combinations thereof. In some embodiments, when a nuclease or a nickase is introduced with a donor template containing an exogenous polynucleotide sequence (also called a transgene) flanked by homology sequences (e.g., homology arms) that are homologous to sequences at or near the endogenous genomic target locus, e.g., a safe harbor locus, DNA damage repair pathways can result in integration of the transgene sequence at the target site in the cell. This can occur by a homology-dependent process. In some embodiments, the donor template is a circular double-stranded plasmid DNA, single-stranded donor oligonucleotide (ssODN), linear double-stranded polymerase chain reaction (PCR) fragments, or the homologous sequences of the intact sister chromatid. Depending on the form of the donor template, the homology-mediated gene insertion and replacement can be carried out via specific DNA repair pathways such as homology-directed repair (HDR), synthesis-dependent strand annealing (SDSA), microhomology-mediated end joining (MMEJ), and homology-mediated end joining (HMEJ) pathways.

For instance, DNA repair mechanisms can be induced by a nuclease after (i) two SSBs, where there is a SSB on each strand, thereby inducing single strand overhangs; or (ii) a DSB occurring at the same cleavage site on both strands, thereby inducing a blunt end break. Upon cleavage by one of these agents, the target locus with the SSBs or the DSB undergoes one of two major pathways for DNA damage repair: (1) the error-prone non-homologous end joining (NHEJ), or (2) the high-fidelity homology-directed repair (HDR) pathway. In some embodiments, a donor template (e.g., circular plasmid DNA or a linear DNA fragment, such as a ssODN) introduced into cells in which there are SSBs or a DSB can result in HDR and integration of the donor template into the target locus. In general, in the absence of a donor template, the NHEJ process re-ligates the ends of the cleaved DNA strands, which frequently results in nucleotide deletions and insertions at the cleavage site.

In some embodiments, site-directed insertion of the exogenous polynucleotide into a cell may be achieved through HDR-based approaches. HDR is a mechanism for cells to repair double-strand breaks (DSBs) in DNA and can be utilized to modify genomes in many organisms using various gene editing systems, including clustered regularly interspaced short palindromic repeat (CRISPR)/Cas systems, zinc finger nucleases (ZFNs), transcription activator-like effector nucleases (TALENs), meganucleases, and transposases.

In some embodiments, the targeted integration is carried by introducing one or more sequence-specific or targeted nucleases, including DNA-binding targeted nucleases and gene editing nucleases such as zinc finger nucleases (ZFN) and transcription activator-like effector nucleases (TALENs), and RNA-guided nucleases such as a CRISPR-associated nuclease (Cas) system, specifically designed to be targeted to at least one target site(s) sequence of a target gene. Exemplary ZFNs, TALEs, and TALENs are described in, e.g., Lloyd et al., Frontiers in Immunology, 4 (221): 1-7 (2013). In particular embodiments, targeted genetic disruption at or near the target site is carried out using clustered regularly interspaced short palindromic repeats (CRISPR) and CRISPR-associated (Cas) proteins. See Sander and Joung, (2014) Nature Biotechnology, 32 (4): 347-355.

### G) ZFNs

ZFNs are fusion proteins comprising an array of site-specific DNA binding domains adapted from zinc finger-containing transcription factors attached to the endonuclease domain of the bacterial FokI restriction enzyme. A ZFN may have one or more (e.g., 1, 2, 3, 4, 5, 6, 7, 8, 9, 10, or more) of the DNA binding domains or zinc finger domains. See, e.g., Carroll et al., Genetics Society of America (2011) 188:773-782; Kim et al., Proc. Natl. Acad. Sci. USA (1996) 93:1156-1160. Each zinc finger domain is a small protein structural motif stabilized by one or more zinc ions and usually recognizes a 3- to 4-bp DNA sequence. Tandem domains can thus potentially bind to an extended nucleotide sequence that is unique within a cell's genome.

Various zinc fingers of known specificity can be combined to produce multi-finger polypeptides which recognize about 6, 9, 12, 15, or 18-bp sequences. Various selection and modular assembly techniques are available to generate zinc fingers (and combinations thereof) recognizing specific sequences, including phage display, yeast one-hybrid systems, bacterial one-hybrid and two-hybrid systems, and mammalian cells. Zinc fingers can be engineered to bind a predetermined nucleic acid sequence. Criteria to engineer a zinc finger to bind to a predetermined nucleic acid sequence are known in the art. See, e.g., Sera et al., Biochemistry (2002) 41:7074-7081; Liu et al., Bioinformatics (2008) 24:1850-1857.

ZFNs containing FokI nuclease domains or other dimeric nuclease domains function as a dimer. Thus, a pair of ZFNs are required to target non-palindromic DNA sites. The two individual ZFNs must bind opposite strands of the DNA with their nucleases properly spaced apart. See Bitinaite et al., Proc. Natl. Acad. Sci. USA (1998) 95:10570-10575. To cleave a specific site in the genome, a pair of ZFNs are designed to recognize two sequences flanking the site, one on the forward strand and the other on the reverse strand. Upon binding of the ZFNs on either side of the site, the nuclease domains dimerize and cleave the DNA at the site, generating a DSB with 5′ overhangs. HDR can then be utilized to introduce a specific mutation, with the help of a repair template containing the desired mutation flanked by homology arms. The repair template is usually an exogenous double-stranded DNA vector introduced to the cell. See Miller et al., Nat. Biotechnol. (2011) 29:143-148; Hockemeyer et al., Nat. Biotechnol. (2011) 29:731-734.

### H) TALENS

TALENs are another example of an artificial nuclease which can be used to edit a target gene. TALENs are derived from DNA binding domains termed TALE repeats, which usually comprise tandem arrays with 10 to 30 repeats that bind and recognize extended DNA sequences. Each repeat is 33 to 35 amino acids in length, with two adjacent amino acids (termed the repeat-variable di-residue, or RVD) conferring specificity for one of the four DNA base pairs. Thus, there is a one-to-one correspondence between the repeats and the base pairs in the target DNA sequences.

TALENs are produced artificially by fusing one or more TALE DNA binding domains (e.g., 1, 2, 3, 4, 5, 6, 7, 8, 9, 10 or more) to a nuclease domain, for example, a FokI endonuclease domain. See Zhang, Nature Biotech. (2011) 29:149-153. Several mutations to FokI have been made for its use in TALENs; these, for example, improve cleavage specificity or activity. See Cermak et al., Nucl. Acids Res. (2011) 39: e82; Miller et al., Nature Biotech. (2011) 29:143-148; Hockemeyer et al., Nature Biotech. (2011) 29:731-734; Wood et al., Science (2011) 333:307; Doyon et al., Nature Methods (2010) 8:74-79; Szczepek et al., Nature Biotech (2007) 25:786-793; Guo et al., J. Mol. Biol. (2010) 200:96. The FokI domain functions as a dimer, requiring two constructs with unique DNA binding domains for sites in the target genome with proper orientation and spacing. Both the number of amino acid residues between the TALE DNA binding domain and the FokI nuclease domain and the number of bases between the two individual TALEN binding sites appear to be important parameters for achieving high levels of activity. Miller et al., Nature Biotech. (2011) 29:143-148.

By combining engineered TALE repeats with a nuclease domain, a site-specific nuclease can be produced specific to any desired DNA sequence. Similar to ZFNs, TALENs can be introduced into a cell to generate DSBs at a desired target site in the genome, and so can be used to knock out genes or knock in mutations in similar, HDR-mediated pathways. See Boch, Nature Biotech. (2011) 29:135-136; Boch et al., Science (2009) 326:1509-1512; Moscou et al., Science (2009) 326:3501.

### I) Meganucleases

Meganucleases are enzymes in the endonuclease family which are characterized by their capacity to recognize and cut large DNA sequences (from 14 to 40 base pairs). Meganucleases are grouped into families based on their structural motifs which affect nuclease activity and/or DNA recognition. The most widespread and best known meganucleases are the proteins in the LAGLIDADG family, which owe their name to a conserved amino acid sequence. See Chevalier et al., Nucleic Acids Res. (2001) 29 (18): 3757-3774. On the other hand, the GIY-YIG family members have a GIY-YIG module, which is 70-100 residues long and includes four or five conserved sequence motifs with four invariant residues, two of which are required for activity. See Van Roey et al., Nature Struct. Biol. (2002) 9:806-811. The His-Cys family meganucleases are characterized by a highly conserved series of histidines and cysteines over a region encompassing several hundred amino acid residues. See Chevalier et al., Nucleic Acids Res. (2001) 29 (18): 3757-3774. Members of the NHN family are defined by motifs containing two pairs of conserved histidines surrounded by asparagine residues. See Chevalier et al., Nucleic Acids Res. (2001) 29 (18): 3757-3774.

Because the chance of identifying a natural meganuclease for a particular target DNA sequence is low due to the high specificity requirement, various methods including mutagenesis and high throughput screening methods have been used to create meganuclease variants that recognize unique sequences. Strategies for engineering a meganuclease with altered DNA-binding specificity, e.g., to bind to a predetermined nucleic acid sequence are known in the art. See, e.g., Chevalier et al., Mol. Cell. (2002) 10:895-905; Epinat et al., Nucleic Acids Res (2003) 31:2952-2962; Silva et al., J Mol. Biol. (2006) 361:744-754; Seligman et al., Nucleic Acids Res (2002) 30:3870-3879; Sussman et al., J Mol Biol (2004) 342:31-41; Doyon et al., J Am Chem Soc (2006) 128:2477-2484; Chen et al., Protein Eng Des Sel (2009) 22:249-256; Arnould et al., J Mol Biol. (2006) 355:443-458; Smith et al., Nucleic Acids Res. (2006) 363 (2): 283-294.

Like ZFNs and TALENs, Meganucleases can create DSBs in the genomic DNA, which can create a frame-shift mutation if improperly repaired, e.g., via NHEJ, leading to a decrease in the expression of a target gene in a cell. Alternatively, foreign DNA can be introduced into the cell along with the meganuclease. Depending on the sequences of the foreign DNA and chromosomal sequence, this process can be used to modify the target gene. See Silva et al., Current Gene Therapy (2011) 11:11-27.

### J) Transposases

Transposases are enzymes that bind to the end of a transposon and catalyze its movement to another part of the genome by a cut and paste mechanism or a replicative transposition mechanism. By linking transposases to other systems such as the CRISPR/Cas system, new gene editing tools can be developed to enable site specific insertions or manipulations of the genomic DNA. There are two known DNA integration methods using transposons which use a catalytically inactive Cas effector protein and Tn7-like transposons. The transposase-dependent DNA integration does not provoke DSBs in the genome, which may guarantee safer and more specific DNA integration.

### K) CRISPR/Cas

The CRISPR system was originally discovered in prokaryotic organisms (e.g., bacteria and archaea) as a system involved in defense against invading phages and plasmids that provides a form of acquired immunity. Now it has been adapted and used as a popular gene editing tool in research and clinical applications.

CRISPR/Cas systems generally comprise at least two components: one or more guide RNAs (gRNAs) and a Cas protein. The Cas protein is a nuclease that introduces a DSB into the target site. CRISPR-Cas systems fall into two major classes: class 1 systems use a complex of multiple Cas proteins to degrade nucleic acids; class 2 systems use a single large Cas protein for the same purpose. Class 1 is divided into types I, III, and IV; class 2 is divided into types II, V, and VI. Different Cas proteins adapted for gene editing applications include, but are not limited to, Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, and Mad7. See, e.g., Jinek et al., Science (2012) 337 (6096): 816-821; Dang et al., Genome Biology (2015) 16:280; Ran et al., Nature (2015) 520:186-191; Zetsche et al., Cell (2015) 163:759-771; Strecker et al., Nature Comm. (2019) 10:212; Yan et al., Science (2019) 363:88-91. The most widely used Cas9 is a type II Cas protein and is described herein as illustrative. These Cas proteins may be originated from different source species. For example, Cas9 can be derived from S. pyogenes or S. aureus.

In the original microbial genome, the type II CRISPR system incorporates sequences from invading DNA between CRISPR repeat sequences encoded as arrays within the host genome. Transcripts from the CRISPR repeat arrays are processed into CRISPR RNAs (crRNAs) each harboring a variable sequence transcribed from the invading DNA, known as the “protospacer” sequence, as well as part of the CRISPR repeat. Each crRNA hybridizes with a second transactivating CRISPR RNA (tracrRNA), and these two RNAs form a complex with the Cas9 nuclease. The protospacer-encoded portion of the crRNA directs the Cas9 complex to cleave complementary target DNA sequences, provided that they are adjacent to short sequences known as “protospacer adjacent motifs” (PAMs).

While the foregoing description has focused on Cas9 nuclease, it should be appreciated that other RNA-guided nucleases exist which utilize gRNAs that differ in some ways from those described to this point. For instance, Cpf1 (CRISPR from Prevotella and Franciscella 1; also known as Cas12a) is an RNA-guided nuclease that only requires a crRNA and does not need a tracrRNA to function.

Since its discovery, the CRISPR system has been adapted for inducing sequence specific DSBs and targeted genome editing in a wide range of cells and organisms spanning from bacteria to eukaryotic cells including human cells. In its use in gene editing applications, artificially designed, synthetic gRNAs have replaced the original crRNA: tracrRNA complexes, including in certain embodiments via a single gRNA. For example, the gRNAs can be single guide RNAs (sgRNAs) composed of a crRNA, a tetraloop, and a tracrRNA. The crRNA usually comprises a complementary region (also called a spacer, usually about 20 nucleotides in length) that is user-designed to recognize a target DNA of interest. The tracrRNA sequence comprises a scaffold region for Cas nuclease binding. The crRNA sequence and the tracrRNA sequence are linked by the tetraloop and each have a short repeat sequence for hybridization with each other, thus generating a chimeric sgRNA. One can change the genomic target of the Cas nuclease by simply changing the spacer or complementary region sequence present in the gRNA. The complementary region will direct the Cas nuclease to the target DNA site through standard RNA-DNA complementary base pairing rules.

In order for the Cas nuclease to function, there must be a PAM immediately downstream of the target sequence in the genomic DNA. Recognition of the PAM by the Cas protein is thought to destabilize the adjacent genomic sequence, allowing interrogation of the sequence by the gRNA and resulting in gRNA-DNA pairing when a matching sequence is present. The specific sequence of PAM varies depending on the species of the Cas gene. For example, the most commonly used Cas9 nuclease derived from S. pyogenes recognizes a PAM sequence of 5′-NGG-3′ or, at less efficient rates, 5′-NAG-3′, where “N” can be any nucleotide. Other Cas nuclease variants with alternative PAMs have also been characterized and successfully used for genome editing, which are summarized in Table 1a.

In some embodiments, Cas nucleases may comprise one or more mutations to alter their activity, specificity, recognition, and/or other characteristics. For example, the Cas nuclease may have one or more mutations that alter its fidelity to mitigate off-target effects (e.g., eSpCas9, SpCas9-HF1, HypaSpCas9, HeFSpCas9, and evoSpCas9 high-fidelity variants of SpCas9). For another example, the Cas nuclease may have one or more mutations that alter its PAM specificity.

In some embodiments, CRISPR systems of the present disclosure comprise TnpB polypeptides. In some embodiments, TnpB polypeptides may comprise a Ruv-C-like domain. The RuvC domain may be a split RuvC domain comprising RuvC-I, RuvC-II, and RuvC-III subdomains. In some embodiments, a TnpB may further comprise one or more of a HTH domain, a bridge helix domain and a zinc finger domain. TnpB polypeptides do not comprise an HNH domain. In some embodiments, a TnpB protein comprises, starting at the N-terminus: a HTH domain, a RuvC-I subdomain, a bridge helix domain, a RuvC-II sub-domain, a zinger finger domain, and a RuvC-III sub-domain. In some embodiments, a RuvC-III sub-domain forms the C-terminus of a TnpB polypeptide. In some embodiments, a TnpB polypeptide is from Epsilonproteobacteria bacterium, Actinoplanes lobatus strain DSM 43150, Actinomadura celluolosilytica strain DSM 45823, Actinomadura namibiensis strain DSM 44197, Alicyclobacillus macrosprangiidus strain DSM 17980, Lipingzhangella halophila strain DSM 102030, or Ktedonobacter recemifer. In some embodiments, a TnpB polypeptide is from Ktedonobacter racemifer, or comprises a conserved RNA region with similarity to the 5′ ITR of K. racemifer TnpB loci. In some embodiments, a TnpB may comprise a Fanzor protein, a TnpB homolog found in eukaryotic genomes. In some embodiments, a CRISPR system comprising a TnpB polypeptide binds a target adjacent motif (TAM) sequence 5′ of a target polynucleotide. In some embodiments, a TAM is a transposon-associated motif. In some embodiments, a TAM sequence comprises TCA. In some embodiments, a TAM sequence comprises TTCAN. In some embodiments, a TAM sequence comprises TTGAT. In some embodiments, a TAM sequence comprises ATAAA.

In certain embodiments, the exogenous polynucleotide may function as a DNA repair template to be integrated into the target site through HDR in associated with a gene editing system (e.g., the CRISPR/Cas system) as described. Generally, the exogenous polynucleotide to be inserted would comprise at least the expression cassette encoding the protein of interest (e.g., the tolerogenic factor) and would optionally also include one or more regulatory elements (e.g., promoters, insulators, enhancers). In certain of these embodiments, the exogenous polynucleotide to be inserted would be flanked by homologous sequence immediately upstream and downstream of the target, i.e., left homology arm (LHA) and right homology arm (RHA), specifically designed for the target genomic locus to serve as template for HDR. The length of each homology arm is generally dependent on the size of the insert being introduced, with larger insertions requiring longer homology arms.

In some embodiments, target-primed reverse transcription (TPRT) or prime editing may be used to engineer exogenous genes, such as exogenous transgenes encoding a tolerogenic factor (e.g., CD47) into specific loci. In some embodiments, prime editing mediates targeted insertions, deletions, all 12 possible base-to-base conversions, and combinations thereof in human cells without requiring DSBs or donor DNA templates.

Prime editing is a genome editing method that directly writes new genetic information into a specified DNA site using a nucleic acid programmable DNA binding protein (“napDNAbp”) working in association with a polymerase (i.e., in the form of a fusion protein or otherwise provided in trans with the napDNAbp), wherein the prime editing system is programmed with a prime editing (PE) guide RNA (“PEgRNA”) that both specifies the target site and templates the synthesis of the desired edit in the form of a replacement DNA strand by way of an extension (either DNA or RNA) engineered onto a guide RNA (e.g., at the 5′ or 3′ end, or at an internal portion of a guide RNA). The replacement strand containing the desired edit (e.g., a single nucleobase substitution) shares the same sequence as the endogenous strand of the target site to be edited (with the exception that it includes the desired edit). Through DNA repair and/or replication machinery, the endogenous strand of the target site is replaced by the newly synthesized replacement strand containing the desired edit. In some cases, prime editing may be thought of as a “search-and-replace” genome editing technology since the prime editors search and locate the desired target site to be edited, and encode a replacement strand containing a desired edit which is installed in place of the corresponding target site endogenous DNA strand at the same time. For example, prime editing can be adapted for conducting precision CRISPR/Cas-based genome editing in order to bypass double stranded breaks. In some embodiments, a homologous protein is or encodes for a Cas protein-reverse transcriptase fusions or related systems to target a specific DNA sequence with a guide RNA, generate a single strand nick at the target site, and use the nicked DNA as a primer for reverse transcription of an engineered reverse transcriptase template that is integrated with the guide RNA. In some embodiments, a prime editor protein is paired with two prime editing guide RNAs (pegRNAs) that template the synthesis of complementary DNA flaps on opposing strands of genomic DNA, resulting in the replacement of endogenous DNA sequence between the PE-induced nick sites with pegRNA-encoded sequences.

In some embodiments, a gene editing technology is associated with a prime editor that is a reverse transcriptase, or any DNA polymerase known in the art. Thus, in one aspect, a prime editor may comprise Cas9 (or an equivalent napDNAbp) which is programmed to target a DNA sequence by associating it with a specialized guide RNA (i.e., PEgRNA) containing a spacer sequence that anneals to a complementary protospacer in the target DNA. Such methods include any disclosed in Anzalone et al., (doi.org/10.1038/s41586-019-1711-4), or in PCT publication Nos. WO2020191248, WO2021226558, or WO2022067130, which are hereby incorporated in their entirety.

In some embodiments, the base editing technology may be used to introduce single-nucleotide variants (SNVs) into DNA or RNA in living cells. Base editing is a CRISPR-Cas9-based genome editing technology that allows the introduction of point mutations in RNAs or DNAs without generating DSBs. Base editors (BEs) are typically fusions of a Cas (“CRISPR-associated”) domain and a nucleobase modification domain (e.g., a natural or evolved deaminase, such as a cytidine deaminase that include APOBEC1 (“apolipoprotein B mRNA editing enzyme, catalytic polypeptide 1”), CDA (“cytidine deaminase”), and AID (“activation-induced cytidine deaminase”)) domains. In some embodiments, base editors may also include proteins or domains that alter cellular DNA repair processes to increase the efficiency and/or stability of the resulting single-nucleotide change. Two major classes of base editors have been developed: cytidine base editors (CBEs) (e.g., BE4) that allow C: G to T: A conversions and adenine base editors (ABEs) (e.g., ABE7.10) that allow A: T to G: C conversions. Base editors are composed by a catalytically dead Cas9 (dCas9) or a nickase Cas9 (nCas9) fused to a deaminase and guided by a sgRNA to the locus of interest. The d/nCas9 recognizes a specific PAM sequence and the DNA unwinds thanks to the complementarity between the sgRNA and the DNA sequence usually located upstream of the PAM (also called protospacer). Then, the opposite DNA strand is accessible to the deaminase that converts the bases located in a specific DNA stretch of the protospacer. Compared to HDR-based strategies, base editing is a promising tool to precisely correct genetic mutations as it avoids gene disruption by NHEJ associated with failed HDR-mediated gene correction. Rat deaminase APOBEC1 (rAPOBEC1) fused to deactivated Cas9 (dCas9) has been used to successfully convert cytidines to thymidines upstream of the PAM of the sgRNA. In some embodiments, this first BE system was optimized by changing the dCas9 to a “nickase” Cas9 D10A, which nicks the strand opposite the deaminated cytidine. Without being bound by theory, this is expected to initiate long-patch base excision repair (BER), where the deaminated strand is preferentially used to template the repair to produce a U:A base pair, which is then converted to T: A during DNA replication.

In some embodiments, a base editor is a nucleobase editor containing a first DNA binding protein domain that is catalytically inactive, a domain having base editing activity, and a second DNA binding protein domain having nickase activity, where the DNA binding protein domains are expressed on a single fusion protein or are expressed separately (e.g., on separate expression vectors). In some embodiments, a base editor is a fusion protein comprising a domain having base editing activity (e.g., cytidine deaminase or adenosine deaminase), and two nucleic acid programmable DNA binding protein domains (napDNAbp), a first comprising nickase activity and a second napDNAbp that is catalytically inactive, wherein at least the two napDNAbp are joined by a linker. In some embodiments, a base editor is a fusion protein that comprises a DNA domain of a CRISPR-Cas (e.g., Cas9) having nickase activity (nCas; nCas9), a catalytically inactive domain of a CRISPR-Cas protein (e.g., Cas9) having nucleic acid programmable DNA binding activity (dCas; e.g., dCas9), and a deaminase domain, wherein the dCas is joined to the nCas by a linker, and the dCas is immediately adjacent to the deaminase domain. In some embodiments, a base editor is an adenine-to-thymine or “ATBE” (or thymine-to-adenine or “TABE”) transversion base editor. Exemplary base editor and base editor systems include any as described in patent publication Nos. US20220127622, US20210079366, US20200248169, US20210093667, US20210071163, WO2020181202, WO2021158921, WO2019126709, WO2020181178, WO2020181195, WO2020214842, WO2020181193, which are hereby incorporated in their entirety.

In some embodiments, a gene editing technology is Programmable Addition via Site-specific Targeting Elements (PASTE). In some aspects, PASTE is platform in which genomic insertion is directed via a CRISPR-Cas9 nickase fused to both a reverse transcriptase and serine integrase. As described in Ioannidi et al. (doi.org/10.1101/2021.11.01.466786), PASTE does not generate double stranded breaks, but allows for integration of sequences as large as ˜36 kb. In some embodiments, a serine integrase can be any known in the art. In some embodiments, a serine integrase has sufficient orthogonality such that PASTE can be used for multiplexed gene integration, simultaneously integrating at least two different genes at at least two genomic loci. In some embodiments, PASTE has editing efficiencies comparable to or better than those of homology directed repair or non-homologous end joining based integration, with activity in non-dividing cells and fewer detectable off-target events.

Any of the systems for gene disruption described herein can be used and, when also introduced with an appropriate donor template having with an exogenous polynucleotide, e.g., transgene sequences, can result in targeted integration of the exogenous polynucleotide at or near the target site of the genetic disruption. In particular embodiments, the genetic disruption is mediated using a CRISPR/Cas system containing one or more guide RNAs (gRNA) and a Cas protein. Exemplary Cas proteins and gRNA are described above, any of which can be used in HDR mediated integration of an exogenous polynucleotide into a target locus to which the Crispr/Cas system is specific for. It is within the level of a skilled artisan to choose an appropriate Cas nuclease and gRNA, such as depending on the particular target locus and target site for cleavage and integration of the exogenous polynucleotide by HDR. Further, depending on the target locus a skilled artisan can readily prepare an appropriate donor template, such as described further below.

In some embodiments, the DNA editing system is an RNA-guided CRISPR/Cas system (such as RNA-based CRISPR/Cas system), wherein the CRISPR/Cas system is capable of creating a double-strand break in the target locus (e.g., safe harbor locus) to induce insertion of the transgene into the target locus. In some embodiments, the nuclease system is a CRISPR/Cas9 system. In some embodiments, the CRISPR/Cas9 system comprises a plasmid-based Cas9. In some embodiments, the CRISPR/Cas9 system comprises a RNA-based Cas9. In some embodiments, the CRISPR/Cas9 system comprises a Cas9 mRNA and gRNA. In some embodiments, the CRISPR/Cas9 system comprises a protein/RNA complex, or a plasmid/RNA complex, or a protein/plasmid complex. In some embodiments, there are provided methods for generating modified cells, which comprises introducing into a source cell (e.g., a pluripotent stem cell, e.g., iPSC) a donor template containing a transgene or exogenous polynucleotide sequence and a DNA nuclease system including a DNA nuclease system (e.g., Cas9) and a locus-specific gRNA. In some embodiments, the Cas9 is introduced as an mRNA. In some embodiments, the Cas9 is introduced as a ribonucleoprotein complex with the gRNA.

Generally, the donor template to be inserted would comprise at least the transgene cassette containing the exogenous polynucleotide of interest (e.g., the tolerogenic factor or CAR) and would optionally also include the promoter. In certain of these embodiments, the transgene cassette containing the exogenous polynucleotide and/or promoter to be inserted would be flanked in the donor template by homology arms with sequences homologous to sequences immediately upstream and downstream of the target cleavage site, i.e., left homology arm (LHA) and right homology arm (RHA). Typically, the homology arms of the donor template are specifically designed for the target genomic locus to serve as template for HDR. The length of each homology arm is generally dependent on the size of the insert being introduced, with larger insertions requiring longer homology arms.

In some embodiments, a donor template (e.g., a recombinant donor repair template) comprises: (i) a transgene cassette comprising an exogenous polynucleotide sequence (for example, a transgene operably linked to a promoter, for example, a heterologous promoter); and (ii) two homology arms that flank the transgene cassette and are homologous to portions of a target locus (e.g. safe harbor locus) at either side of a DNA nuclease (e.g., Cas nuclease, such as Cas9 or Cas12) cleavage site. The donor template can further comprise a selectable marker, a detectable marker, and/or a purification marker.

In some embodiments, the homology arms are the same length. In other embodiments, the homology arms are different lengths. The homology arms can be at least about 10 base pairs (bp), e.g., at least about 10 bp, 15 bp, 20 bp, 25 bp, 30 bp, 35 bp, 45 bp, 55 bp, 65 bp, 75 bp, 85 bp, 95 bp, 100 bp, 150 bp, 200 bp, 250 bp, 300 bp, 350 bp, 400 bp, 450 bp, 500 bp, 550 bp, 600 bp, 650 bp, 700 bp, 750 bp, 800 bp, 850 bp, 900 bp, 950 bp, 1000 bp, 1.1 kilobases (kb), 1.2 kb, 1.3 kb, 1.4 kb, 1.5 kb, 1.6 kb, 1.7 kb, 1.8 kb, 1.9 kb, 2.0 kb, 2.1 kb, 2.2 kb, 2.3 kb, 2.4 kb, 2.5 kb, 2.6 kb, 2.7 kb, 2.8 kb, 2.9 kb, 3.0 kb, 3.1 kb, 3.2 kb, 3.3 kb, 3.4 kb, 3.5 kb, 3.6 kb, 3.7 kb, 3.8 kb, 3.9 kb, 4.0 kb, or longer. The homology arms can be about 10 bp to about 4 kb, e.g., about 10 bp to about 20 bp, about 10 bp to about 50 bp, about 10 bp to about 100 bp, about 10 bp to about 200 bp, about 10 bp to about 500 bp, about 10 bp to about I kb, about 10 bp to about 2 kb, about 10 bp to about 4 kb, about 100 bp to about 200 bp, about 100 bp to about 500 bp, about 100 bp to about 1 kb, about 100 bp to about 2 kb, about 100 bp to about 4 kb, about 500 bp to about I kb, about 500 bp to about 2 kb, about 500 bp to about 4 kb, about 1 kb to about 2 kb, about 1 kb to about 2 kb, about 1 kb to about 4 kb, or about 2 kb to about 4 kb.

In some embodiments, the donor template can be cloned into an expression vector. Conventional viral and non-viral based expression vectors known to those of ordinary skill in the art can be used.

In some embodiments, the target locus targeted for integration may be any locus in which it would be acceptable or desired to target integration of an exogenous polynucleotide or transgene. Non-limiting examples of a target locus include, but are not limited to, a CXCR4 gene, an albumin gene, a SHS231 locus, an F3 gene (also known as CD142), a MICA gene, a MICB gene, a LRP1 gene (also known as CD91), a HMGB1 gene, an ABO gene, a RHD gene, a FUT1 gene, a KDM5D gene (also known as HY), a B2M gene, a CIITA gene, a CCR5 gene, a F3 (i.e., CD142) gene, a MICA gene, a MICB gene, a LRP1 gene, a HMGB1 gene, an ABO gene, a RHD gene, a FUT1 gene, a KDM5D (i.e., HY) gene, a PDGFRa gene, a OLIG2 gene, and/or a GFAP gene. In some embodiments, the exogenous polynucleotide can be inserted in a suitable region of the target locus (e.g., safe harbor locus), including, for example, an intron, an exon, and/or gene coding region (also known as a Coding Sequence, or “CDS”). In some embodiments, the insertion occurs in one allele of the target genomic locus. In some embodiments, the insertion occurs in both alleles of the target genomic locus. In either of these embodiments, the orientation of the transgene inserted into the target genomic locus can be either the same or the reverse of the direction of the gene in that locus.

In some embodiments, the exogenous polynucleotide is interested into an intron, exon, or coding sequence region of the safe harbor gene locus. In some embodiments, the exogenous polynucleotide is inserted into an endogenous gene wherein the insertion causes silencing or reduced expression of the endogenous gene. Exemplary genomic loci for insertion of an exogenous polynucleotide are depicted in Table 2.

TABLE 2
Exemplary genomic loci for insertion of exogenous polynucleotides

| | | | | Target region | |
|---|---|---|---|---|---|
| Number | species | name | Ensembl ID | for cleavage | Also known as |
| 1 | human | B2M | ENSG00000166710 | CDS | |
| 2 | human | CIITA | ENSG00000179583 | CDS | |
| 3 | human | TRAC | ENSG00000277734 | CDS | |
| 4 | human | PPP1R12C | ENSG00000125503 | Intron 1 and 2 | AAVS1 |
| 5 | human | CLYBL | ENSG00000125246 | Intron 2 | |
| 6 | human | CCR5 | ENSG00000160791 | Exons 1-3, | |
| | | | | introns 1-2, | |
| | | | | and CDS | |
| 7 | human | THUMPD3-AS1 | ENSG00000206573 | Intron 1 | ROSA26 |
| 8 | human | Ch- 4: 58, 976, 613 | | 500 bp window | SHS231 |
| 9 | human | F3 | ENSG00000117525 | CDS | CD142 |
| 10 | human | MICA | ENSG00000204520 | CDS | |
| 11 | human | MICB | ENSG00000204516 | CDS | |
| 12 | human | LRP1 | ENSG00000123384 | CDS | |
| 13 | human | HMGB1 | ENSG00000189403 | CDS | |
| 14 | human | ABO | ENSG00000175164 | CDS | |
| 15 | human | RHD | ENSG00000187010 | CDS | |
| 16 | human | FUT1 | ENSG00000174951 | CDS | |
| 17 | human | KDM5D | ENSG00000012817 | CDS | HY |

In some embodiments, the target locus is a safe harbor locus. In some embodiments, a safe harbor locus is a genomic location that allows for stable expression of integrated DNA with minimal impact on nearby or adjacent endogenous genes, regulatory element and the like. In some cases, a safe harbor gene enables sustainable gene expression and can be targeted by engineered nuclease for gene modification in various cell types including pluripotent stem cells, including derivatives thereof, and differentiated cells thereof. Non-limiting examples of a safe harbor locus include, but are not limited to, a CCR5 gene locus, a PPP1R12C (also known as AAVS1) gene locus, a CLYBL gene locus, and/or a Rosa gene locus (e.g., ROSA26 gene locus). n some embodiments, the safe harbor locus is selected from the group consisting of the AAVS1 locus, the CCR5 locus, and the CLYBL locus. In some cases SHS231 can be targeted as a safe harbor locus in many cell types. In some cases, certain loci can function as a safe harbor locus in certain cell types. For instance, PDGFRa is a safe harbor for glial progenitor cells (GPCs), OLIG2 is a safe harbor locus for oligodendrocytes, and GFAP is a safe harbor locus for astrocytes. It is within the level of a skilled artisan to choose an appropriate safe harbor locus depending on the particular modified cell type. In some cases, more than one safe harbor gene can be targeted, thereby introducing more than one transgene into the genetically modified cell.

In some embodiments, there are provided methods for generating modified cells, which comprises introducing into a source cell (e.g. a pluripotent stem cell, e.g. iPSC) a donor template containing a transgene or exogenous polynucleotide sequence and a DNA nuclease system including a DNA nuclease system (e.g. Cas9) and a locus-specific gRNA that comprise complementary portions (e.g. gRNA targeting sequence) specific to a CCR5 gene locus, a PPP1R12C (also known as AAVS1) gene locus, a CLYBL gene locus, and/or a Rosa gene locus (e.g., ROSA26 gene locus). In some embodiments, the genomic locus targeted by the gRNAs is located within 4000 bp, within 3500 bp, within 3000 bp, within 2500 bp, within 2000 bp, within 1500 bp, within 1000 bp, or within 500 bp of any of the loci as described.

In some embodiments, the gRNAs used herein for HDR-mediated insertion of a transgene comprise a complementary portion (e.g. gRNA targeting sequence) that recognizes a target sequence in AAVS1. In certain of these embodiments, the target sequence is located in intron 1 of AAVS 1. AAVS1 is located at Chromosome 19:55,090,918-55,117,637 reverse strand, and AAVS1 intron 1 (based on transcript ENSG00000125503) is located at Chromosome 19:55,117,222-55,112,796 reverse strand. In certain embodiments, the gRNAs target a genomic locus within 4000 bp, within 3500 bp, within 3000 bp, within 2500 bp, within 2000 bp, within 1500 bp, within 1000 bp, or within 500 bp of Chromosome 19:55, 117,222-55, 112,796. In certain embodiments, the gRNAs target a genomic locus within 4000 bp, within 3500 bp, within 3000 bp, within 2500 bp, within 2000 bp, within 1500 bp, within 1000 bp, or within 500 bp of Chromosome 19:55,115,674. In certain embodiments, the gRNA is configured to produce a cut site at Chromosome 19:55, 115,674, or at a position within 5, 10, 15, 20, 30, 40 or 50 nucleotides of Chromosome 19:55, 115,674. In certain embodiments, the gRNA s GET000046, also known as “sgAAVS1-1,” described in Li et al., Nat. Methods 16:866-869 (2019). This gRNA comprises a complementary portion (e.g., gRNA targeting sequence) having the nucleic acid sequence set forth in SEQ ID NO: 36 (e.g., Table 3) and targets intron 1 of AAVS1 (also known as PPP1R12C).

In some embodiments, the gRNAs used herein for HDR-mediated insertion of a transgene comprise a complementary portion (e.g., gRNA targeting sequence) that recognizes a target sequence in CLYBL. In certain of these embodiments, the target sequence is located in intron 2 of CL YBL. CLYBL is located at Chromosome 13:99,606,669-99,897, 134 forward strand, and CLYBL intron 2 (based on transcript ENST00000376355.7) is located at Chromosome 13:99,773,011-99,858,860 forward strand. In certain embodiments, the gRNAs target a genomic locus within 4000 bp, within 3500 bp, within 3000 bp, within 2500 bp, within 2000 bp, within 1500 bp, within 1000 bp, or within 500 bp of Chromosome 13:99, 773,011-99,858,860. In certain embodiments, the gRNAs target a genomic locus within 4000 bp, within 3500 bp, within 3000 bp, within 2500 bp, within 2000 bp, within 1500 bp, within 1000 bp, or within 500 bp of Chromosome 13:99,822,980. In certain embodiments, the gRNA is configured to produce a cut site at Chromosome 13:99,822,980, or at a position within 5, 0, 15, 20, 30, 40 or 50 nucleotides of Chromosome 13:99,822,980. In certain embodiments, the gRNA is GET000047, which comprises a complementary portion (e.g., gRNA targeting sequence) having the nucleic acid sequence set forth in SEQ ID NO: 36 (e.g., Table 3) and targets intron 2 of CLYBL. The target site is similar to the target site of the TALENs as described in Cerbini et al., PLoS One, 10 (1): e0116032 (2015).

In some embodiments, the gRNAs used herein for HDR-mediated insertion of a transgene comprise a complementary portion (e.g., gRNA targeting sequence) that recognizes a target sequence in CCR5. In certain of these embodiments, the target sequence is located in exon 3 of CCR5. CCR5 is located at Chromosome 3:46,370,854-46,376,206 forward strand, and CCR5 exon 3 (based on transcript ENST00000292303.4) is located at Chromosome 3:46,372,892-46,376,206 forward strand. In certain embodiments, the gRNAs target a genomic locus within 4000 bp, within 3500 bp, within 3000 bp, within 2500 bp, within 2000 bp, within 1500 bp, within 1000 bp, or within 500 bp of Chromosome 3:46, 372,892-46,376,206. In certain embodiments, the gRNAs target a genomic locus within 4000 bp, within 3500 bp, within 3000 bp, within 2500 bp, within 2000 bp, within 1500 bp, within 1000 bp, or within 500 bp of Chromosome 3:46,373,180. In certain embodiments, the gRNA is configured to produce a cut site at Chromosome 3:46,373,180, or at a position within 5, 10, 15, 20, 30, 40, or 50 nucleotides of Chromosome 3:46,373,180. In certain embodiments the gRNA is GET000048, also known as “crCCR5_D,” described in Mandal et al., Cell Stem Cell 15:643-652 (2014). This gRNA comprises a complementary portion having the nucleic acid sequence set forth in SEQ ID NO: 37 (e.g., Table 3) and targets exon 3 of CCR5 (alternatively annotated as exon 2 in the Ensembl genome database). See Gomez-Ospina et al., Nat. Comm. 10 (1): 4045 (2019).

Table 3 sets forth exemplary gRNA targeting sequences. In some embodiments, the gRNA targeting sequence may contain one or more thymines in the complementary portion sequences set forth in Table 3 are substituted with uracil.

| | | SEQ |
|---|---|---|
| Description | Nucleic Acid Sequence | ID NO: |
| GET000046 | (5′→3′) | 35 |
| guide | accccacagtggggccacta | |
| GET000047 | (5′→3′) | 36 |
| guide | tgttggaaggatgaggaaat | |
| G ET000048 | (5′→3′) | 37 |
| guide | tcactatgctgccgcccagt | |

In some embodiments, the target locus is a locus that is desired to be knocked out in the cells. In such embodiments, such a target locus is any target locus whose disruption or elimination is desired in the cell, such as to modulate a phenotype or function of the cell. For instance, any of the gene modifications described herein to reduce expression of a target gene may be a desired target locus for targeted integration of an exogenous polynucleotide, in which the genetic disruption or knockout of a target gene and overexpression by targeted insertion of an exogenous polynucleotide may be achieved at the same target site or locus in the cell. For instance, the HDR process may be used to result in a genetic disruption to eliminate or reduce expression of (e.g. knock out) any target gene set forth in Table 1a or Table 1b while also integrating (e.g. knocking in) an exogenous polynucleotide into the target gene by using a donor template with flanking homology arms that are homologous to nucleic acid sequences at or near the target site of the genetic disruption.

In some embodiments, there are provided methods for generating modified cells, which comprises introducing into a source cell (e.g., a pluripotent stem cell, e.g. iPSC) a donor template containing a transgene or exogenous polynucleotide sequence and a DNA nuclease system including a DNA nuclease system (e.g. Cas9) and a locus-specific gRNA that comprise complementary portions specific to the B2M locus or the CIITA locus. In some embodiments, the genomic locus targeted by the gRNAs is located within 4000 bp, within 3500 bp, within 3000 bp, within 2500 bp, within 2000 bp, within 1500 bp, within 1000 bp, or within 500 bp of any of the loci as described.

In particular embodiments, the target locus is B2M. In some embodiments, the modified cell comprises a genetic modification targeting the B2M gene. In some embodiments, the genetic modification targeting the B2M gene is by using a targeted nuclease system that comprises a Cas protein or a polynucleotide encoding a Cas protein, and at least one guide ribonucleic acid sequence for specifically targeting the B2M gene. In some embodiments, the at least one guide ribonucleic acid (gRNA) sequence for specifically targeting the B2M gene is selected from the group consisting of SEQ ID NOS: 81240-85644 of Appendix 2 or Table 15 of WO2016/183041, the disclosure is incorporated by reference in its entirety. In some embodiments, an exogenous polynucleotide is integrated into the disrupted B2M locus by HDR by introducing a donor template containing the exogenous polynucleotide sequence with flanking homology arms homologous to sequences adjacent to the target site targeted by the gRNA.

In particular embodiments, the target locus is CIITA. In some embodiments, the modified cell comprises a genetic modification targeting the CIITA gene. In some embodiments, the genetic modification targeting the CIITA gene is by a targeted nuclease system that comprises a Cas protein or a polynucleotide encoding a Cas protein, and at least one guide ribonucleic acid sequence for specifically targeting the CIITA gene. In some embodiments, the at least one guide ribonucleic acid sequence for specifically targeting the CIITA gene is selected from the group consisting of SEQ ID NOS: 5184-36352 of Appendix 1 or Table 12 of WO2016183041, the disclosure is incorporated by reference in its entirety. In some embodiments, an exogenous polynucleotide is integrated into the disrupted CIITA locus by HDR by introducing a donor template containing the exogenous polynucleotide sequence with flanking homology arms homologous to sequences adjacent to the target site targeted by the gRNA.

In some embodiments, it is within the level of a skilled artisan to identify new loci and/or gRNA sequences for use in HDR-mediated integration approaches as described. For example, for CRISPR/Cas systems, when an existing gRNA for a particular locus (e.g., within a target gene, e.g. set forth in Table 1b or Table 1c) is known, an “inch worming” approach can be used to identify additional loci for targeted insertion of transgenes by scanning the flanking regions on either side of the locus for PAM sequences, which usually occurs about every 100 base pairs (bp) across the genome. The PAM sequence will depend on the particular Cas nuclease used because different nucleases usually have different corresponding PAM sequences. The flanking regions on either side of the locus can be between about 500 to 4000 bp long, for example, about 500 bp, about 1000 bp, about 1500 bp, about 2000 bp, about 2500 bp, about 3000 bp, about 3500 bp, or about 4000 bp long. When a PAM sequence is identified within the search range, a new guide can be designed according to the sequence of that locus for use in genetic disruption methods. Although the CRISPR/Cas system is described as illustrative, any HDR-mediated approaches as described can be used in this method of identifying new loci, including those using ZFNs, TALENS, meganucleases and transposases.

In some embodiments, the exogenous polynucleotide encodes an exogenous CD47 polypeptide (e.g., a human CD47 polypeptide) and the exogenous polypeptide is inserted into a safe harbor gene loci or a safe harbor site as disclosed herein or a genomic locus that causes silencing or reduced expression of the endogenous gene. In some embodiments, the exogenous polynucleotide encoding CD47 is inserted in a CCR5 gene locus, a PPP1R12C (also known as AAVS1) gene locus, a CLYBL gene locus, and/or a Rosa gene locus (e.g., ROSA26 gene locus). In some embodiments, the polynucleotide is inserted in a B2M, CIITA, PD1 or CTLA4 gene locus.

In some embodiments, the modified cell that includes the exogenous polynucleotide is a beta islet cell and includes a first exogenous polynucleotide that encodes a CD47 polypeptide. In some embodiments, the modified pluripotent stem cells (e.g. modified iPSC) further comprises one or more additional exogenous polynucleotides that encode one or more complement inhibitors or other tolerogenic polypeptides described herein. In some embodiments, the modified pluripotent stem cells (e.g. modified iPSC) comprises reduced expression of CD142 and reduced expression of MHC class I and/or reduced expression of MHC class II. In some embodiments, the first exogenous polynucleotide and the one or more additional exogenous polynucleotide are inserted into the same genomic locus. In some embodiments, the first exogenous polynucleotide and the one or more additional exogenous polynucleotide are inserted into different genomic loci. In exemplary embodiments, the modified (e.g., hypoimmunogenic) cell is a beta islet cell derived from an modified (e.g., hypoimmunogenic) pluripotent cell (e.g., an iPSC).

In some embodiments, the cell is a beta islet cell. In some embodiments, the cell is an iPSC-derived cell that has been differentiated from a modified iPSC. In some embodiments, the cell comprises reduced or eliminated expression of CD142. In some embodiments, the cell comprises overexpression or increased expression of one or more complement inhibitor.

In some embodiments, the cell is an iPSC-derived beta-islet cell that is modified to contain modifications (e.g. genetic modifications) described herein. In some embodiments, the cell comprises reduced or eliminated expression of CD142. In some embodiments, the cell comprises overexpression or increased expression of one or more complement inhibitor. In some embodiments, the modified (e.g. hypoimmunogenic) beta-islet cell can be used to treat a variety of indications with allogenic cell therapy, including any as described herein. In some embodiments, the modified (e.g. hypoimmunogenic) beta-islet cell can be used to treat diabetes, such as type I diabetes.

In some embodiments, the cells that are modified as provided herein are cells from a healthy subject, such as a subject that is not known or suspected of having a particular disease or condition to be treated.

### II. Modified Stem Cell-Derived Beta Cell and Methods of Generating Modified Stem Cell-Derived Beta Cells

Provided herein are modified stem cell-derived beta (modified SC-beta) cells obtained by in vitro differentiation of a pluripotent stem cell.

In some embodiments, provided herein are modified stem cell-derived beta (modified SC-beta) cells obtained by in vitro differentiation of a modified pluripotent stem cell. The modified pluripotent stem cell can be any as described above, e.g. Section I. The provided modified SC-beta cells are differentiated in vitro from the modified pluripotent stem cell by any method able to generate a functional SC-beta cell. In some of any embodiments, the differentiated modified SC-beta cell is a modified iPSC-derived beta islet cell. In some of any embodiments, the differentiated modified SC-beta cell is an ESC-derived cell. The provided modified SC-beta cells retain the one or more modifications of the modified pluripotent stem cells and/or retain or exhibit similar expression of the target immune molecules (e.g. reduced expression of MHC class I and/or II and increased expression of a tolerogenic factor, such as CD47). The modified SC-beta cells provided herein also are functional and exhibit one or more functions of primary beta cells or beta islet cells, such as the ability to secrete insulin, for example glucose stimulated insulin secretion (GSIS).

In some embodiments, also provided herein are modified stem cell-derived beta (modified SC-beta) cells obtained by in vitro differentiation of a pluripotent stem cell to generate an SC-beta cell, and introduction of the modifications into the SC-beta cell. The modifications introduced in the modified SC-beta cell can be any of the modifications described in Section I (B) for modified PSCs. The provided modified SC-beta cells are differentiated in vitro from the pluripotent stem cell by any method able to generate a functional SC-beta cell, and modified to generate the modified SC-beta cell. In some of any embodiments, the differentiated modified SC-beta cell is an iPSC-derived beta islet cell. In some of any embodiments, the differentiated modified SC-beta cell is an ESC-derived cell. The modified SC-beta cells provided herein also are functional and exhibit one or more functions of primary beta cells or beta islet cells, such as the ability to secrete insulin, for example glucose stimulated insulin secretion (GSIS).

In some embodiments, the modified stem-cell derived beta cell (SC-beta cell) comprises one or more modifications that: (a) inactivate or disrupt one or more alleles of: (i) one or more major histocompatibility complex (MHC) class I molecules or one or more molecules that regulate expression of the one or more MHC class I molecules, and/or (ii) one or more MHC class II molecules or one or more molecules that regulate expression of the one or more MHC class II molecules, and/or (b) increase expression of one or more tolerogenic factors, wherein the increased expression is relative to a control or wild-type beta cell that does not comprise the modifications. The one or more modifications can be introduced into the SC-beta cell according to any of the methods for inactivating or disrupting genes and/or for overexpression of polynucleotides described in Sections I.B.1 and I.B.2 above for modified PSCs.

Also provided are populations of cells containing the modified beta cells. It is understood that differentiation from a population may not result in 100% having fully differentiated to the same stage in the differentiation pathway. Thus, it should be appreciated that not all cells in a particular population progress through these stages at the same rate, i.e., some cells may have progressed less, or more, down the differentiation pathway than the majority of cells present in the population. Accordingly, a population of beta-cells (e.g. having a b cell marker) may also include cells that are partially differentiated from the modified pluripotent stem cell or is a precursor of the cell stage such as precursor of the differentiated SC-beta cell. In some cases, a percentage or portion of the cells may be at an earlier stage. Exemplary features of provided populations are provided in Section III.

In some embodiments, the modified SC-beta cells are differentiated in vitro (e.g., from pluripotent stem cells) and are cells that display at least one marker indicative of a pancreatic beta cell (e.g., PDX-1 or NKX6-1), express insulin, and display a GSIS response characteristic of an endogenous mature beta cell both in vitro and in vivo. In some embodiments, a marker indicative of a beta cell is a marker selected from INS, CHGA, NKX2-2, PDX1, NKX6-1, MAFB, GCK and GLUT1. In some embodiments, the GSIS response of the modified SC-beta cell can be observed within two weeks of transplantation of the SC-beta cell into a host (e.g., a human or animal). In some embodiments, it is to be understood that the SC-beta cells need not be derived (e.g., directly) from stem cells, as any method can be used that is capable of deriving SC-beta cells from any endocrine progenitor cell that expresses insulin or precursor thereof using any cell as a starting point in which such starting cell has been modified by the one or more modifications described herein.

In some embodiments, the starting cell may be a cell according to the present disclosure that is an embryonic stem cells, induced-pluripotent stem cells, progenitor cells, partially reprogrammed somatic cells (e.g., a somatic cell which has been partially reprogrammed to an intermediate state between an induced pluripotent stem cell and the somatic cell from which it was derived), multipotent cells, totipotent cells, a transdifferentiated version of any of the foregoing cells. In some embodiments, the starting cell does not comprise the one or more modifications that (a) inactivate or disrupt one or more alleles of: (i) one or more major histocompatibility complex (MHC) class I molecules or one or more molecules that regulate expression of the one or more MHC class I molecules, and/or (ii) one or more MHC class II molecules or one or more molecules that regulate expression of the one or more MHC class II molecules, and/or (b) increase expression of one or more tolerogenic factors.

In some embodiments, the starting cell may be a modified cell according to the present disclosure that is an embryonic stem cells, induced-pluripotent stem cells, progenitor cells, partially reprogrammed somatic cells (e.g., a somatic cell which has been partially reprogrammed to an intermediate state between an induced pluripotent stem cell and the somatic cell from which it was derived), multipotent cells, totipotent cells, a transdifferentiated version of any of the foregoing cells.

In some embodiments, the modified SC-beta cells have regulated or modulated (e.g. reduced or eliminated) expression of MHC class I molecules, MHC class II molecules, or MHC class I and MHC class II molecules. In some embodiments, the regulated or modulated expression of MHC class I and/or Class II is due to gene editing in which the DNA of the gene loci involved in regulation of expression of MHC class I and/or class II have been edited to delete genomic DNA of a gene involved in regulation of expression of the immune molecule. In some embodiments, the modified SC-beta cell has an edit to delete genomic DNA of beta-2 microglobulin (B2M) and is thus reduced or eliminated for expression of MHC class I. In some embodiments, the B2M gene is knocked out in the modified SC-beta cell. In some embodiments, both alleles of B2M are knocked out. In some embodiments, the modified SC-beta cell has an edit to delete genomic DNA of CIITA and is thus reduced or eliminated for expression of MHC class II. In some embodiments, the CIITA gene is knocked out in the modified SC-beta cell. In some embodiments, both alleles of CIITA are knocked out.

In some embodiments, the modified SC-beta cells have regulated or modulated (e.g. increase) expression of a tolerogenic factor, such as CD47. In some embodiments, the tolerogenic factor is one or more of DUX4, B2M-HLA-E, CD16, CD52, CD47, CD27, CD200, HLA-C, HLA-E, HLA-E heavy chain, HLA-G, PD-L1, IDO1, CTLA4-Ig, Cl-Inhibitor, IL-10, IL-35, FASL, CCL21, MFGE8, SERPINB9, CD35, IL-39, CD16 Fc Receptor, IL15-RF, and H2-M3, or any combination thereof. In some embodiments, the one or more tolerogenic factors are selected from the group consisting of CD16, CD24, CD35, CD39, CD46, CD47, CD52, CD55, CD59, CD64, CD200, CCL22, CTLA4-Ig, C1 inhibitor, FASL, IDO1, HLA-C, HLA-E, HLA-E heavy chain, HLA-G, IL-10, IL-35, PD-L1, SERPINB9, CCL21, MFGE8, DUX4, B2M-HLA-E, CD27, IL-39, CD16 Fc Receptor, IL15-RF, H2-M3 (HLA-G), A20/TNFAIP3, CR1, HLA-F, and MANF. In some embodiments, the increased or overexpressed tolerogenic factor is or includes increased expression of CCL21, PD-L1, FasL, Serpinb9, H2-M3 (HLA-G), CD47, CD200, and Mfge8. In some embodiments, the tolerogenic factor is CD47 and the modified SC-beta cell has increased expression of CD47. In some embodiments, the tolerogenic factor is PD-L1 and the modified SC-beta cell includes increased expression of PD-L1. In some embodiments, the tolerogenic factor is HLA-E and the modified SC-beta cell includes increased expression of HLA-E. In some embodiments, the tolerogenic factor is HLA-G and the modified beta-cell includes increased expression of HLA-G. In some embodiments, the tolerogenic factor is expressed as an exogenous polynucleotide or transgene in the genome of the modified SC-beta cell. In some embodiments, the exogenous polynucleotide or transgene is integrated or inserted into a genome locus of the cells, such as a safe harbor locus. In some embodiments, the genomic locus is an ABO, CCR5, CLYBL, CXCR4, F3, FUT1, HMGB1, KDM5D, LRP1, MICA, MICB, RHD, ROSA26, or SHS231 locus.

In some aspects, provided are modified SC-beta cell (e.g. iPSC-derived beta islet cell) having (1) reduced expression of MHC I and/or MHC II; and (2) a transgene comprising CD47 and a safety switch inserted at a safe harbor locus, wherein the safe harbor locus is selected from the group consisting of an AAVS1, ABO, CCR5, CLYBL, CXCR4, F3, FUT1, HMGB1, KDM5D, LRP1, MICA, MICB, RHD, ROSA26, and SHS231 locus. In some aspects, provided are modified pluripotent stem cells having (1) reduced expression of MHC I and/or MHC II; and (2) a transgene comprising CD47 and HSVtk flanked by CLYBL homology arms, wherein the transgene is inserted at the CLYBL locus. In some embodiments, the modified pluripotent stem cell has B2M and/or CIITA knockout. In some embodiments, the B2M and/or CIITA knockout occur in both alleles.

In some aspects, provided are ESC-derived stem cell having (1) reduced expression of MHC I and/or MHC II; and (2) a transgene comprising CD47 and a safety switch inserted at a safe harbor locus, wherein the safe harbor locus is selected from the group consisting of an AAVS1, ABO, CCR5, CLYBL, CXCR4, F3, FUT1, HMGB1, KDM5D, LRP1, MICA, MICB, RHD, ROSA26, and SHS231 locus. In some aspects, provided are ESC-derived cells having (1) reduced expression of MHC I and/or MHC II; and (2) a transgene comprising CD47 and HSVtk flanked by CLYBL homology arms, wherein the transgene is inserted at the CLYBL locus. In some embodiments, the ESC-derived cell has B2M and/or CIITA knockout. In some embodiments, the B2M and/or CIITA knockout occur in both alleles.

In some embodiments, a modified SC-beta cell provided herein comprises a safety switch. The introduction of safety switches improves the safety of cell therapies developed using hypoimmunogenic cells (HIP cells, e.g., modified SC-beta cells). In some embodiments, feature of the HIP cells described herein is the inducible expression of one or more immune regulatory (immunosuppressive) factors In some embodiments, an immunosuppressive factor (also referred to herein as “an hypoimmunity factor”) includes, but is not limited to, CD47, CD24, CD200, HLA-G, HLA-E, HLA-C, HLA-E heavy chain, PD-L1, IDO1, CTLA4-Ig, Cl-Inhibitor, IL-10, IL-35, FASL, Serpmb9, CCl21, and Mfge8. In certain embodiments, the immunosuppressive factor is CD47. The regulatable or inducible expression of an immunosuppressive factor functions to control an immune response by a recipient subject to an engrafted hypoimmunogenic cell.

Described herein are methods for the expression of an immunosuppressive factor that requires a mechanism to ‘turn-off expression of the immune regulatory protein in a controlled manner. Also described are modified SC-beta cells possessing controllable expression of one or more immunosuppressive factors. In some cases, the cells overexpress one or more immunosuppressive factors and can be induced to downregulate expression of the one or more immunosuppressive factors. As such, the cells are no longer hypoimmunogenic and are recognized by the recipient's immune cells for cell death.

In some embodiments, the hypoimmunity of the modified SC-beta cells that are introduced to a recipient subject is achieved through the overexpression of an immunosuppressive molecule including hypoimmunity factors and complement inhibitors accompanied with the repression or genetic disruption of the HLA-I and HLA-II loci. These modifications cloak the cell from the recipient immune system's effector cells that are responsible for the clearance of infected, malignant or non-self cells, such as T cells, B cells, NK cells and macrophages. Cloaking of a cell from the immune system allows for existence and persistence of allogeneic cells within the body. Controlled removal of the engineered cells from the body is crucial for patient safety and can be achieved by uncloaking the cells from the immune system. Uncloaking serves as a safety switch and can be achieved through the downregulation of the immunosuppressive molecules or the upregulation of immune signaling molecules. The level of expression of any of the immunosuppressive molecules described can be controlled on the protein level, mRNA level, or DNA level in the cells. Similarly, the level of expression of any of the immune signaling molecules described can be controlled on the protein level, mRNA level, or DNA level in the cells.

In some embodiments, any of the safety switch methods described (e.g., protein level, RNA level and DNA level safety switches) are used to decrease the level of an immunosuppressive factor in the cells such that the lower level of the immunosuppressive factor is below a threshold level. In some embodiments, the level of the immunosuppressive factor in the cells is decreased by about 10-fold, 9-fold, 8-fold, 7-fold, 6-fold, 5-fold, 4-fold, 3-fold, 2-fold, 1-fold or 0.5-fold below a threshold level of expression. In some embodiments, the level of the immunosuppressive factor in the cells is decreased by about 10-fold to 5-fold, 10-fold to 3-fold, 9-fold to 1-fold, 8-fold to 1-fold, 7-fold to 0.5-fold, 6-fold, to 1-fold, 5-fold to 0.5-fold, 4-fold to 0.5-fold, 3-fold to 0.5-fold, 2-fold to 0.5-fold, or 1-fold to 0.5-fold below a threshold level of expression. In some embodiments, the threshold level of expression of the immunosuppressive factor is established based on the expression of such factor in an induced pluripotent stem cell. In some embodiments, the threshold level of the immunosuppressive factor expression is established based on the expression level of the immunosuppressive factor in a corresponding hypoimmune cell, such as any of the modified SC-beta cells described herein.

In some embodiments, transcriptional regulation of immunosuppressive factors through employing inducible promoters provides the ability to turn expression of the switch on or off at will through the addition or removal of small molecules, such as, but not limited to, doxycycline. Genetic disruption via targeted nuclease activity can eliminate expression of the immunosuppressive factor to uncloak the cells as well. Exemplary safety switches are described in WO2021146627A1, the content of which is herein incorporated by reference in its entirety.

In some embodiments, any of the above modified SC-beta cells further have regulated or modulated (e.g. reduced or eliminated) expression of CD142. In some embodiments, the regulated or modulated expression of CD142 is due to gene editing in which the DNA of the CD142 gene loci has been edited to delete genomic DNA. In some embodiments, the modified SC-beta cell has an edit to delete genomic DNA of CD142 and is thus reduced or eliminated for expression of CD142. In some embodiments, the CD142 gene is knocked out in the modified SC-beta cell. In some embodiments, both alleles of B2M are knocked out.

In some embodiments, any of the above modified SC-beta cells further have regulated or modulated (e.g. increased) expression of one or more complement inhibitor. In some embodiments, the one or more complement inhibitors is any one of CD46, CD59 and CD55 or is a combination thereof (e.g. CD46 and CD59 or CD46, CD59 and CD55). In some embodiments, the one or more complement inhibitor is expressed as an exogenous polynucleotide(s) or transgene(s) in the genome of the modified SC-beta cell. In some embodiments, the exogenous polynucleotide(s) or transgene(s) is integrated or inserted into a genome locus of the cells, such as a safe harbor locus. In some embodiments, the genomic locus is an ABO, CCR5, CLYBL, CXCR4, F3, FUT1, HMGB1, KDM5D, LRP1, MICA, MICB, RHD, ROSA26, or SHS231 locus. In some embodiments, the exogenous polynucleotide or transgene is expressed at the same or a different locus from CD47 and/or from a suicide gene.

### A. Differentiation of Stem-Cell Derived Beta Cells

In some embodiments, the methods used to differentiate the stem cell-derived β cells (SC-β) are known by one skilled in the art. Such methods are described, for example, in WO2019018818, U.S. Pat. Nos. 8,507,274, 10,030,229, 10,190,096, 10,253,298, 10,443,042, WO2016100925, WO2019217493, U.S. Pat. Nos. 7,510,876, 8,216,836, 8,633,024, 8,647,873, 10,421,942, 9,404,086, US20190359943, U.S. Pat. Nos. 10,358,628, 8,633,024, 8,647,873, 9,222,069, 10,465,162, 10,370,645, 9,725,699, 10,253,298, 9,499,795, 9,650,610, 9,062,290, 10,494,609, US20210060083, U.S. Pat. Nos. 8,129,182, 8,603,811, 9,328,331, 9,012,218, 9,109,245, 9,982,235, 9,988,604, 10,358,628, 10,138,465, US20190211309, U.S. Pat. No. 10,443,042, WO2020207998A1, US20210230554A1, US20200308548A1, US20190085295A1, US20200002670A1, US20190153394A1, U.S. Pat. No. 10,487,313, US20120135519A1, US20210207099, Pagliuca et al, (Cell 2014), and Hogrebe et al. (2021), all of which are herein incorporated by reference.

In some embodiments, the process of differentiating pluripotent stem cells into functional pancreatic endocrine cells (i.e., SC-beta cells) in vitro may be viewed in some aspects as progressing through six consecutive stages. In some embodiments, stage 1 refers to the first step in the differentiation process, the differentiation of pluripotent stem cells into cells expressing markers characteristic of definitive endoderm cells. Stage 2 refers to the second step, the differentiation of cells expressing markers characteristic of definitive endoderm cells into cells expressing markers characteristic of gut tube cells. Stage 3 refers to the third step, the differentiation of cells expressing markers characteristic of gut tube cells into cells expressing markers characteristic of early pancreas progenitor cells. Stage 4 refers to the fourth step, the differentiation of cells expressing markers characteristic of early pancreas progenitor cells into cells expressing markers characteristic of pancreatic progenitor cell. Stage 5 refers to the fifth step, the differentiation of cells expressing markers characteristic of pancreatic progenitor cells into cells expressing markers characteristic of pancreatic endoderm cells and/or pancreatic endocrine progenitor cells. It should be appreciated, however, that not all cells in a particular population progress through these stages at the same rate, i.e., some cells may have progressed less, or more, down the differentiation pathway than the majority of cells present in the population. Thus, it is understood that in any step reference to a particular stage may include contacting of the particular cell of a given stage with a compound where cells in the contacted population may include a cell that is partially differentiated from the modified pluripotent stem cell or is a precursor of the cell stage.

In some embodiments, a definitive endoderm cell is a cell that bears the characteristics of cells arising from the epiblast during gastrulation and which form the gastrointestinal tract and its derivatives. Definitive endoderm cells express at least one of the following markers: FOXA2 (also known as hepatocyte nuclear factor 3β (“HNF3β”)), GATA4, SOX17, CXCR4, Brachyury, Cerberus, OTX2, goosecoid, C-Kit, CD99, and MIXL1. Markers characteristic of the definitive endoderm cells include CXCR4, FOXA2 and SOX17. Thus, definitive endoderm cells may be characterized by their expression of CXCR4, FOXA2 and SOX17. In addition, depending on the length of time cells are allowed to remain in Stage 1, an increase in HNF4α may be observed.

In some embodiments, gut tube cells are cells derived from definitive endoderm that can give rise to all endodermal organs, such as lungs, liver, pancreas, stomach, and intestine. Gut tube cells may be characterized by their substantially increased expression of HNF4α over that expressed by definitive endoderm cells. For example, a ten to forty fold increase in mRNA expression of HNF4α may be observed during Stage 2.

In some embodiments, early pancreas progenitor cells refer to endoderm cells that give rise to the esophagus, lungs, stomach, liver, pancreas, gall bladder, and a portion of the duodenum. Early pancreatic progenitor cells express at least one of the following markers: PDX1, FOXA2, CDX2, SOX2, and HNF4α. Early pancreatic progenitor cells may be characterized by an increase in expression of PDX1, compared to gut tube cells. For example, greater than fifty percent of the cells in Stage 3 cultures typically express PDX1.

In some embodiments, pancreatic progenitor cells refer to cells that express at least one of the following markers: PDX1, NKX6.1, HNF6, NGN3, SOX9, PAX4, PAX6, ISL1, gastrin, FOXA2, PTF1a, PROX1 and HNF4α. Pancreatic progenitor cells may be characterized as positive for the expression of PDX1, NKX6.1, and SOX9.

In some embodiments, a pancreatic endoderm cell (also sometimes called a pancreatic endocrine progenitor cells) is a cell capable of becoming a pancreatic hormone expressing cell. Pancreatic endoderm cells express at least one of the following markers: NGN3; NKX2.2; NeuroD1; ISL1; PAX4; PAX6; or ARX. Pancreatic endoderm cells may be characterized by their expression of NKX2.2 and NeuroD1.

Provided herein is a method of generating insulin-producing beta cells comprising: providing a stem cell (e.g. modified stem cell, such as modified iPSC); providing serum-free media; contacting the stem cell with a TGF/Activin agonist or a glycogen synthase kinase 3 (GSK) inhibitor or WNT agonist for an amount of time sufficient to form a definitive endoderm cell; contacting the definitive endoderm cell with a FGFR2b agonist for an amount of time sufficient to form a primitive gut tube cell; contacting the primitive gut tube cell with an RAR agonist, and optionally a rho kinase inhibitor, a Smoothened antagonist, a FGFR2b agonist, a protein kinase C activator, or a BMP type 1 receptor inhibitor for an amount of time sufficient to form an early pancreas progenitor cell; incubating the early pancreas progenitor cell for at least about 3 days and optionally contacting the early pancreas progenitor cell with a rho kinase inhibitor, a TGF-/Activin agonist, a Smoothened antagonist, an FGFR2b agonist, or a RAR agonist for an amount of time sufficient to form a pancreatic progenitor cell; contacting the pancreatic progenitor cell with an Alk5 inhibitor, a gamma secretase inhibitor, a Smoothened antagonist (e.g., SANT1), an Erbb1 (EGFR) or Erbb4 agonist, or a RAR agonist for an amount of time sufficient to form an endoderm cell; or resizing cell clusters within about 24 hours and allowing the endoderm cell to mature for an amount of time in serum-free media sufficient to form a beta cell.

Provided herein is a method of generating insulin-producing beta cells comprising: providing a stem cell (e.g. modified stem cell, such as modified iPSC, or a stem cell that does not comprise one or more modifications); providing serum-free media; contacting the stem cell with a TGF/Activin agonist and/or a glycogen synthase kinase 3 (GSK) inhibitor and/or WNT agonist for an amount of time sufficient to form a definitive endoderm cell; contacting the definitive endoderm cell with a FGFR2b agonist for an amount of time sufficient to form a primitive gut tube cell; contacting the primitive gut tube cell with an RAR agonist, a rho kinase inhibitor, a Smoothened antagonist, a FGFR2b agonist, a protein kinase C activator, and/or a BMP type 1 receptor inhibitor for an amount of time sufficient to form an early pancreas progenitor cell; incubating the early pancreas progenitor cell for at least about 3 days and optionally contacting the early pancreas progenitor cell with a rho kinase inhibitor, a TGF-/Activin agonist, a Smoothened antagonist, an FGFR2b agonist, and/or a RAR agonist for an amount of time sufficient to form a pancreatic progenitor cell; contacting the pancreatic progenitor cell with an Alk5 inhibitor, a gamma secretase inhibitor, a Smoothened antagonist (e.g., SANT1), an Erbb1 (EGFR) and/or Erbb4 agonist, and/or a RAR agonist for an amount of time sufficient to form an endoderm cell; and resizing cell clusters within about 24 hours and allowing the endoderm cell to mature for an amount of time in serum-free media sufficient to form a beta cell.

In some embodiments, the serum-free media comprises one or more selected from the group consisting of: MCDB131, glucose, NaHCOs, BSA, ITS-X, Glutamax, vitamin C, penicillin-streptomycin, CMRL 10666, FBS, Heparin, NEAA, trace elements A, trace elements B, or ZnS0₄.

In some embodiments, the TGF/Activin agonist is Activin A; the glycogen synthase kinase 3 (GSK) inhibitor or the WNT agonist is CHIR; the FGFR2b agonist is KGF; the Smoothened antagonist or hedgehog pathway inhibitor is SANT-1; the FGF family member/FGFR2b agonist is KGF; the RAR agonist is RA; the protein kinase 3 activator is TPPB; the BMP inhibitor is LDN; the rho kinase inhibitor is Y27632; the Alk5 inhibitor/TGF-b receptor inhibitor is Alk5i; the thyroid hormone is T3; or the gamma secretase inhibitor is XXI.

In some embodiments, the TGF/Activin agonist is Activin A. In certain embodiments, the concentration of Activin A is between 50 ng/ml-150 ng/ml. In certain embodiments, the concentration of Activin A is 50 ng/ml, 55 ng/ml, 60 ng/ml, 65 ng/ml, 70 ng/ml, 75 ng/ml, 80 ng/ml, 85 ng/ml, 90 ng/ml, 95 ng/ml, 100 ng/ml, 105 ng/ml, 110 ng/ml, 115 ng/ml, 120 ng/ml, 125 ng/ml, 130 ng/ml, 135 ng/ml, 140 ng/ml, 145 ng/ml, or 150 ng/ml. In certain embodiments, the concentration of Activin A is between 50 ng/ml-60 ng/ml, 55 ng/ml-65 ng/ml, 60 ng/ml-70 ng/ml, 65 ng/ml-75 ng/ml, 70 ng/ml-80 ng/ml, 75 ng/ml-85 ng/ml, 80 ng/ml-90 ng/ml, 85 ng/ml-95 ng/ml, 90 ng/ml-100 ng/ml, 95 ng/ml-105 ng/ml, 100 ng/ml-110 ng/ml, 105 ng/ml-115 ng/ml, 110 ng/ml-120 ng/ml, 115 ng/ml-125 ng/ml, 120 ng/ml-130 ng/ml, 125 ng/ml-135 ng/ml, 130 ng/ml-140 ng/ml, 135 ng/ml-145 ng/ml, or 140 ng/ml-150 ng/ml. In a specific embodiment, the concentration of Activin A is 100 ng/ml.

In some embodiments, the glycogen synthase kinase 3 (GSK) inhibitor or the WNT agonist is CHIR. In certain embodiments, the concentration of CHIR is between 0.5 μM and 5 μM. In certain embodiments, the concentration of the CHIR is 0.5 μM, 1.0 μM, 1.5 μM, 2.0 μM, 2.5 μM, 3.0 μM, 3.5 μM, 4.0 μM, 4.5 μM, or 5.0 μM. In certain embodiments, the concentration of CHIR is between 0.5 μM-1.5 μM, 1.0 μM-2.0 μM, 1.5 μM-2.5 μM, 2.0 μM-3.0 μM, 2.5 μM-3.5 μM, 3.0 μM-4.0 μM, 3.5 μM-4.5 μM, or 4.0 μM-5.0 μM. In a specific embodiment, the concentration of CHIR is 3.0 μM.

In certain embodiments, the FGFR2b agonist is KGF. In certain embodiments, the concentration of KGF is between 5 ng/ml-100 ng/ml. In certain embodiments, the concentration of KGF is 5 ng/ml, 10 ng/ml, 15 ng/ml, 20 ng/ml, 25 ng/ml, 30 ng/ml, 35 ng/ml, 40 ng/ml, 45 ng/ml, 50 ng/ml, 55 ng/ml, 60 ng/ml, 65 ng/ml, 70 ng/ml, 75 ng/ml, 80 ng/ml, 85 ng/ml, 90 ng/ml, 95 ng/ml, or 100 ng/ml. In certain embodiments, the concentration of KGF is between 5 ng/ml-15 ng/ml, 10 ng/ml-20 ng/ml, 15 ng/ml-25 ng/ml, 20 ng/ml-30 ng/ml, 25 ng/ml-35 ng/ml, 30 ng/ml-40 ng/ml, 35 ng/ml-45 ng/ml, 40 ng/ml-50 ng/ml, 45 ng/ml-55 ng/ml, 50 ng/ml-60 ng/ml, 55 ng/ml-65 ng/ml, 60 ng/ml-70 ng/ml, 65 ng/ml-75 ng/ml, 70 ng/ml-80 ng/ml, 75 ng/ml-85 ng/ml, 80 ng/ml-90 ng/ml, 85 ng/ml-95 ng/ml, 90 ng/ml-100 ng/ml. In a specific embodiment, the concentration of the KGF is 50 ng/ml.

In certain embodiments, the Smoothened antagonist is SANT-1. In certain embodiments, the concentration of SANT-1 is between 0.05 μM and 0.50 μM. In certain embodiments, the concentration of the SANT-1 is 0.05 μM, 0.10 μM, 0.15 μM, 0.20 μM, 0.25 μM, 0.3 μM, 0.35 μM, 0.4 μM, 0.45 μM, or 0.5 μM. In certain embodiments, the concentration of SANT-1 is between 0.05 μM-0.15 μM, 0.10 μM-0.20 μM, 0.15 μM-0.25 μM, 0.20 μM-0.30 μM, 0.25 μM-0.35 μM, 0.30 μM-0.40 μM, 0.35 μM-0.45 μM, or 0.40 μM-0.50 μM. In a specific embodiment, the concentration of SANT-1 is 0.25 μM.

In certain embodiments, the RAR agonist is retinoic acid (RA). In certain embodiments, the concentration of RA is between 0.05 μM and 2.5 μM. In certain embodiments, the concentration of RA is 0.05 μM, 0.1 μM, 0.15 μM, 0.2 μM, 0.5 μM, 1.0 μM, 1.5 μM, 2.0 μM, or 2.5 μM. In certain embodiments, the concentration of RA is between 0.005 μM-0.15 μM, 0.10 μM-0.2 μM, 0.15 μM-0.5 μM, 0.2 μM-1.0 μM, 0.5 μM-1.5 μM, 1.0 μM-2.0 μM, or 1.5 μM-2.5 μM. In a specific embodiment, the concentration of RA is 0.10 μM. In a specific embodiment, the concentration of RA is 2.0 μM.

In some embodiments, the protein kinase C activator is TPPB. In certain embodiments, the concentration of TPPB is between 0.05 μM and 0.50 μM. In certain embodiments, the concentration of the TPPB is 0.05 μM, 0.10 μM, 0.15 μM, 0.20 μM, 0.25 μM, 0.3 μM, 0.35 μM, 0.4 μM, 0.45 μM, or 0.5 μM. In certain embodiments, the concentration of TPPB is between 0.05 μM-0.15 μM, 0.10 μM-0.20 μM, 0.15 μM-0.25 μM, 0.20 μM-0.30 μM, 0.25 μM-0.35 μM, 0.30 μM-0.40 μM, 0.35 μM-0.45 μM, or 0.40 μM-0.50 μM. In a specific embodiment, the concentration of TPPB is 0.20 μM.

In some embodiments, the BMP type 1 receptor inhibitor is LDN193189. In certain embodiments, the concentration of LDN193189 is between 0.05 μM and 0.50 μM. In certain embodiments, the concentration of the LDN193189 is 0.05 μM, 0.10 μM, 0.15 μM, 0.20 μM, 0.25 μM, 0.3 μM, 0.35 μM, 0.4 μM, 0.45 μM, or 0.5 μM. In certain embodiments, the concentration of LDN193189 is between 0.05 μM-0.15 μM, 0.10 μM-0.20 μM, 0.15 μM-0.25 μM, 0.20 μM-0.30 μM, 0.25 μM-0.35 μM, 0.30 μM-0.40 μM, 0.35 μM-0.45 μM, or 0.40 μM-0.50 μM. In a specific embodiment, the concentration of LDN193189 is 0.20 μM.

In some embodiments, the Alk5 inhibitor is Alk5i. In certain embodiments, the concentration of Alk5i is between 5.0 μM and 15 μM. In certain embodiments, the concentration of Alk5i is 5.0 μM, 6.0 μM, 7.0 μM, 8.0 μM, 9.0 μM, 10.0 μM, 11.0 μM, 12.0 μM, 13.0 μM, 14.0 μM, or 15.0 μM. In certain embodiments, the concentration of Alk5i is between 5.0 μM-7.0 μM, 6.0 μM-8.0 μM, 7.0 μM-9.0 μM, 8.0 μM-10.0 μM, 9.0 μM-11.0 μM, 10.0 μM-12.0 μM, 11.0 μM-13.0 μM, 12.0 μM-14.0 μM, or 13.0 μM-15.0 μM. In a specific embodiment, the concentration of Alk5i is 10.0 μM.

In certain embodiments, latrunculin A is utilized to chemically depolymerize the actin cytoskeleton. In certain embodiments, the concentration of latrunculin A is 0.5 μM and 1.5 μM. In certain embodiments, the concentration of latrunculin A is 0.5 μM, 0.6 μM, 0.7 μM, 0.8 μM, 0.9 μM, 1.0 μM, 1.1 μM, 1.2 μM, 1.3 μM, 1.4 μM, or 1.5 μM. In certain embodiments, the concentration of latrunculin A is between 0.5 μM-0.7 μM, 0.6 μM-0.8 μM, 0.7 μM-0.9 μM, 0.8 μM-1.0 μM, 0.9 μM-1.1 μM, 1.0 μM-1.2 μM, 1.1 μM-1.3 μM, 1.2 μM-1.4 μM, or 1.3 μM-1.5 μM. In a specific embodiment, the concentration of latrunculin A is 1.0 μM.

In certain embodiments, the thyroid hormone is T3. In certain embodiments, the concentration of T3 is between 0.1 μM and 2 μM. In certain embodiments, the concentration of T3 is 0.1 μM, 0.2 μM, 0.3 μM, 0.4 μM, 0.5 μM, 0.6 μM, 0.7 μM, 0.8 μM, 0.9 μM, 1.0 μM, 1.1 μM, 1.2 μM, 1.3 μM, 1.4 μM, 1.5 μM, 1.6 μM, 1.7 μM, 1.8 μM, 1.9 μM, or 2.0 μM. In certain embodiments, the concentration of T3 is between 0.1 μM-0.3 μM, 0.2 μM-0.4 μM, 0.3 μM-0.5 μM, 0.4 μM-0.6 μM, 0.5 μM-0.7 μM, 0.6 μM-0.8 μM, 0.7 μM-0.9 μM, 0.8 μM-1.0 μM, 0.9 μM-1.1 μM, 1.0 μM-1.2 μM, 1.1 μM-1.3 μM, 1.2 μM-1.4 μM, 1.3 μM-1.5 μM, 1.4 μM-1.6 μM, 1.5 μM-1.7 μM, 1.6 μM-1.8 μM, 1.7 μM-1.9 μM, or 1.8 μM-2.0 μM. In a specific embodiment, the concentration of T3 is 1.0 μM.

In certain embodiments, the gamma secretase inhibitor is XXI. In certain embodiments, the concentration of XXI is between 0.1 μM and 2 μM. In certain embodiments, the concentration of XXI is 0.1 μM, 0.2 μM, 0.3 μM, 0.4 μM, 0.5 μM, 0.6 μM, 0.7 μM, 0.8 μM, 0.9 μM, 1.0 μM, 1.1 μM, 1.2 μM, 1.3 μM, 1.4 μM, 1.5 μM, 1.6 μM, 1.7 μM, 1.8 μM, 1.9 μM, or 2.0 μM. In certain embodiments, the concentration of XXI is between 0.1 μM-0.3 μM, 0.2 μM-0.4 μM, 0.3 μM-0.5 μM, 0.4 μM-0.6 μM, 0.5 μM-0.7 μM, 0.6 μM-0.8 μM, 0.7 μM-0.9 μM, 0.8 μM-1.0 μM, 0.9 μM-1.1 μM, 1.0 μM-1.2 μM, 1.1 μM-1.3 μM, 1.2 μM-1.4 μM, 1.3 μM-1.5 μM, 1.4 μM-1.6 μM, 1.5 μM-1.7 μM, 1.6 μM-1.8 μM, 1.7 μM-1.9 μM, or 1.8 μM-2.0 μM. In a specific embodiment, the concentration of XXI is 1.0 μM.

In certain embodiments, the methods herein detail a differentiation protocol for generating highly functional SC-β cells. The methods provided herein comprise six stages that attempt to recreate phases of pancreatic organogenesis by activating and repressing specific developmental pathways with growth factors and small molecules in serum-free medium. In certain embodiments, to propagate and expand cells for SC-β-cell differentiation, hPSCs are seeded onto Matrigel-coated TCP plates at a density of 0.8×105 cells/cm2 and cultured in medium.

In certain embodiments, the methods provided herein comprise six stages of stem cell differentiation. In certain embodiments, Stage 1 comprises incubating a HPSC of Stage 0 in media comprising Activin A and CHIR for about 24 hours followed by about 3 days of incubating the cells in media comprising Activin A in the absence of CHIR. In certain embodiments, Stage 2 comprises incubating the Stage 1 cells for 2 days in media comprising KGF. In some embodiments, the CHIR is CHIR99021.

In certain embodiments, Stage 3 comprises incubating Stage 2 cells for 2 days in media comprising KGF, LDN193189, TPPB, RA (high), and SANT1. In certain embodiments, Stage 4 comprises incubating Stage 3 cells for about 4 days in media comprising KGF, LDN193189, TPPB, RA (low), and SANT1. In certain embodiments, Stage 5 comprises incubating the Stage 4 cells in media comprising XXI, Alk5i, T3, SANT1, and RA for 7 days. Additionally, latrunculin A is added to the media for about the first 24 hours of incubation. In certain embodiments, Stage 6 comprises incubating the cells in an enriched serum-free medium which allows the SC-β cells the time needed to mature before they become glucose responsive. The methods provided herein generate stem cell-derived beta (SC-β) cells that function better (undergoing glucose-stimulated insulin secretion) than cells in the published literature (Pagliuca et al. Cell 2014) and express beta cell markers.

In some embodiments, the amount of time sufficient to form a definitive endoderm cell, a primitive gut tube cell, an early pancreas progenitor cell, a pancreatic progenitor cell, an endoderm cell, or a beta cell is between about 1 day and about 15 days.

As described herein, stem cell-derived beta (SC-β) cells can be useful as a cellular therapy for diabetes. The presently disclosed method enhances differentiation of human pluripotent stem cells to insulin-producing beta cells. This process is modified from a previously described 6-step differentiation protocol published by Pagliuca et al. Cell 2014. Using the methods disclosed herein, cells that can respond to glucose appropriately to near islet-like levels have been generated, demonstrating both a first phase and second phase response. In order to achieve the above modulation, the following was performed: (1) shorten stage 3 to 1 day; (2) allow for TGFbeta signaling in stage 6 by removal of Alk5 inhibitor II (3) remove T3 from stage 6; (4) perform stage 6 in a serum-free basal media; and (5) break apart and reaggregate clusters at the beginning of stage 6.

Using the above modulations, enhanced stem cell-derived beta cells that better perform glucose-stimulated insulin secretion were generated. The field currently includes Alk5 inhibitor II and T3 during the last stage of culture to mature stem cell-derived beta cells. The field has been unable to generate functional stem cell-derived beta cells that have both first phase and second phase insulin secretion (see Rezania et al. Nature Biotechnology 2014 for the poor dynamic function stem cell-derived beta cells have in the field).

### 1. Modulation of the Actin Cytoskeleton

As described herein, the actin cytoskeleton is a crucial regulator of human pancreatic cell fate. By controlling the state of the cytoskeleton with either cell arrangement (two-vs three-dimensional), substrate stiffness, or directly with chemical treatment, a polymerized cytoskeleton prevents premature induction of NEUROG3 expression in pancreatic progenitors, but also inhibits subsequent differentiation to SC-β cells.

Modulation of the actin cytoskeleton and its downstream effector Yes-Associated Protein (YAP) at specific time points during differentiation can enhance differentiation of human pluripotent stem cells to cells of endodermal lineage, pancreatic progenitors, and insulin-producing beta cells. Using the 6-stage differentiation protocol modified from Pagliuca et al. Cell 2014, the following specific features were observed: (1) actin polymerization and YAP activity during Stage 4 enhances generation of pancreatic progenitors (PDX1+/NKX6-1+/SOX9+); (2) actin depolymerization and loss of YAP activity during Stage 5, preferentially during the first 24-48 hr of Stage 5, enhances generation of endocrine cells, specifically beta cells that demonstrate enhanced glucose-stimulated insulin secretion (WO2019/222487).

In order to achieve the above modulation, the following can be performed: (1) promoting actin polymerization by plating onto stiff surfaces, such as tissue culture plastic with a thin layer of ECM protein to promote attachment; (2) promoting actin depolymerization by plating onto soft surfaces, such as hydrogels, or by treating cells with latrunculin A and/or latrunculin B; (3) promoting YAP transcriptional activity using the same methods to promote actin polymerization; and/or (4) inhibiting YAP transcriptional activity using the same methods to promote actin depolymerization or by treatment with Verteporfin.

Using the above modulations, enhanced stem cell-derived beta cells were generated to better perform glucose-stimulated insulin secretion than previous methods and can be generated on attachment culture. Currently in the field, stem cell-derived beta cells can be generated but do not function as well as with the presently disclosed approach. The field does not utilize actin cytoskeleton and YAP signaling in their protocols. The field is also unable to generate functional stem cell-derived beta cells with the cells in attachment culture—it must either be done in suspension aggregates (the control for many experiments in the attached data set, first reported in Pagliuca et al. Cell 2014) or in aggregates on an air-liquid-interface (first reported in Rezania et al. Nature Biotechnology 2014).

Described herein is the generation of stem cell-derived beta cells that function better (undergoing glucose-stimulated insulin secretion) than cells in the published literature (Pagliuca et al. Cell 2014) and express beta cell markers. Also described herein are methods for the generation of stem cell-derived beta cells in a planar protocol that can undergo glucose-stimulated insulin secretion (GSIS).

Described herein is the demonstration that cells can be detached from a plate, either using UpCell technology that does not require cell dispersion or by dispersing and reaggregating the cells, and maintain insulin secretion capacity, better enabling transplantation. Also described herein is the generation of pancreatic progenitor cells that have reduced endocrine expression (such as expression of NGN3, NEUROD1) and increased pancreatic progenitor expression (such as expression of NKX6-1, SOX9).

Pancreatic progenitors and stem cell-derived beta cells can be useful as a cellular therapy for diabetes. The presently disclosed culture approach can also facilitate enhanced quality and reproducibility of the differentiations and is conducive to automation of the differentiation process for commercialization. In an example, differentiation protocols by cytoskeletal modulation can generate cells of several lineages (e.g., SC-b, beta-like cells). It was discovered that the state of the actin cytoskeleton is critical to endodermal cell fate choice. By utilizing a combination of cell-biomaterial interactions as well as small molecule regulators of the actin cytoskeleton (e.g., a cytoskeletal-modulating agent), the timing of endocrine transcription factor expression can be controlled to modulate differentiation fate and develop a two-dimensional protocol for differentiating cells. Importantly, this new planar protocol greatly enhances the function of SC-b cells differentiated from induced pluripotent stem cell (iPSC) lines and forgoes the requirement for three-dimensional cellular arrangements.

Different degrees of actin polymerization at specific points of differentiation biased cells toward different endodermal lineages, and thus non-optimal cytoskeletal states led to large inefficiencies in cell specification. Furthermore, the methods described herein can control actin polymerization to direct differentiations of these other endodermal cell fates to modulate lineage specification. Other lineages that can be generated according to the provided methods can be liver, esophageal, exocrine, pancreas, intestine, or stomach.

A cytoskeletal-modulating agent can be any agent that promotes or inhibits actin polymerization or microtubule polymerization. For example, the cytoskeletal-modulating agent can be an actin depolymerization or polymerization agent, a microtubule modulating agent, or an integrin modulating agent (e.g., compounds, such as antibodies and small molecules). For example, the cytoskeletal-modulating agent can be latrunculin A, latrunculin B, nocodazole, cytochalasin D, jasplakinolide, blebbistatin, y-27632, y-15, gdc-0994, or an integrin modulating agent. The cytoskeletal-modulating agent can be any cytoskeletal-modulating agent known in the art (see e.g., Ley et al. Nat Rev Drug Discov. 2016 March; 15 (3): 173-183).

### 2. Cell Cluster Resizing

Resizing of cell clusters can be performed by any methods known in the art. For example, cell resizing can comprise breaking apart cell clusters and reaggregating. As another example, the cell clusters can be resized by incubating in a cell-dissociating reagent and passed through a cell strainer (e.g., a 100 pm nylon cell strainer). As another example, cells can be resized by single cell dispersing with TrypLE and reaggregating.

In certain embodiments, a rotational shaker may be used to induce clustering and reaggregation of these cells. In certain embodiments, mostly endocrine cells aggregate together at this point in the differentiation after cell fate has already been specified in planar culture, while nonendocrine cell types generated during this protocol tend to die off. In certain embodiments, a reaggregation step may facilitate endocrine purification without the need for a more expensive and timeconsuming sorting procedure.

### B. Inactivation or Disruption of Target Genes

### 1. Target Genes

In some embodiments, any of the described modifications in the modified SC-beta cells that regulate (e.g., reduce or eliminate) expression of one or more target polynucleotide or protein in the modified SC-beta cells may be combined with one or more modifications to overexpress a polynucleotide (e.g., tolerogenic factor, such as CD47). The modifications in the modified SC-beta cells that regulate (e.g., reduce or eliminate) expression of one or more target polynucleotide or protein in the modified SC-beta cells can be any of the modifications and described in Section I.B.1 above for modified PSCs. The methods of inacticating or disrupting genes in the modified SC-beta cell can be any of the methods described in Section I.B.1 above for modified PSCs.

### 2. Overexpression of Polynucleotides

In some embodiments, the modified SC-beta cells provided herein are genetically modified, such as by introduction of one or more modifications into a cell to overexpress a desired polynucleotide in the cell. In some embodiments, the cell to be modified is an unmodified cell that has not previously been introduced with the one or more modifications. In some embodiments, the cell to be modified is an SC-beta cell. In some embodiments, the modified SC-beta cells provided herein are genetically modified to include one or more exogenous polynucleotides encoding an exogenous protein (also interchangeably used with the term “transgene”). As described, in some embodiments, the cells are modified to increase expression of certain genes that are tolerogenic (e.g., immune) factors that affect immune recognition and tolerance in a recipient. In some embodiments, the provided modified cells, such as T cells or NK cells, also express a chimeric antigen receptor (CAR). The one or more polynucleotides, e.g., exogenous polynucleotides, may be expressed (e.g. overexpressed) in the modified SC-beta cells together with one or more genetic modifications to reduce expression of a target polynucleotide described above, such as an MHC class I and/or MHC class II molecule or CD142. In some embodiments, the provided modified SC-beta cells do not trigger or activate an immune response upon administration to a recipient subject.

In some embodiments, the modified SC-beta cell includes 1, 2, 3, 4, 5, 6, 7, 8, 9, 10 or more different overexpressed polynucleotides. In some embodiments, the overexpressed polynucleotide is an exogenous polynucleotide. In some embodiments, the modified SC-beta cell includes 1, 2, 3, 4, 5, 6, 7, 8, 9, 10 or more different exogenous polynucleotides. In some embodiments, the overexpressed polynucleotide is an exogenous polynucleotide that is expressed episomally in the cells. In some embodiments, the overexpressed polynucleotide is an exogenous polynucleotide that is inserted or integrated into one or more genomic loci of the modified cell.

In some embodiments, expression of a polynucleotide is increased, i.e., the polynucleotide is overexpressed, using a fusion protein containing a DNA-targeting domain and a transcriptional activator. Targeted methods of increasing expression using transactivator domains are known to a skilled artisan.

In some embodiments, the modified SC-beta cell contains one or more exogenous polynucleotides in which the one or more exogenous polynucleotides are inserted or integrated into a genomic locus of the cell by non-targeted insertion methods, such as by transduction with a lentiviral vector. In some embodiments, the one or more exogenous polynucleotides are inserted or integrated into the genome of the cell by targeted insertion methods, such as by using homology directed repair (HDR). Any suitable method can be used to insert the exogenous polynucleotide into the genomic locus of the modified cell by HDR including the gene editing methods described herein (e.g., a CRISPR/Cas system). In some embodiments, the one or more exogenous polynucleotides are inserted into one or more genomic locus, such as any genomic locus described herein (e.g., Table 2). In some embodiments, the exogenous polynucleotides are inserted into the same genomic loci. In some embodiments, the exogenous polynucleotides are inserted into different genomic loci. In some embodiments, the two or more of the exogenous polynucleotides are inserted into the same genomic loci, such as any genomic locus described herein (e.g., Table 2). In some embodiments, two or more exogenous polynucleotides are inserted into a different genomic loci, such as two or more genomic loci as described herein (e.g., Table 2).

Exemplary polynucleotides or overexpression, and methods for overexpressing the same, include any of those described for modified PSCs in Section I.B.2 above.

### C. Exemplary Features of the SC-β Cells

The provided modified SC-beta cells, including those obtained by in vitro differentiation from pluripotent stem cells such as modified pluripotent stem cells, are highly functional SC-β cells. The modified SC-beta cell or population exhibits a GSIS response both in vitro and in vivo. The isolated SC-beta cell or population also exhibits at least one characteristic feature of a mature endogenous beta cell. In some aspects, a modified SC-beta cell or population thereof exhibits a stimulation index of between about 1.4 and about 2.4. In some aspects, a modified SC-beta cell or population thereof produces insulin at between approximately 300 uIU to about 4000 uIU per 30 minute per 106 total cells incubation at a high glucose concentration.

In certain embodiments, static insulin secretion is greater than about 1 uIU/103 cells/hour at high glucose (e.g., 20 mM). In certain embodiments, the static insulin secretion is greater than about 1.5 uIU/103 cells/hour at high glucose. In certain embodiments, the static insulin secretion is greater than about 2.0 uIU/103 cells/hour at high glucose. In certain embodiments, the static insulin secretion is greater than about 2.5 uIU/103 cells/hour at high glucose. In certain embodiments, the static insulin secretion is greater than about 3.0 uIU/103 cells/hour at high glucose. In certain embodiments, the static insulin secretion is greater than about 3.5 uIU/103 cells/hour at high glucose. In certain embodiments, the static insulin secretion is greater than about 4.0 uIU/103 cells/hour at high glucose. In certain embodiments, the static stimulation index is defined as a ratio of 20 mM glucose to 2 mM glucose, incubated for 1 hr.

Assays to assess functional activity of the SC-b cells include static and dynamic glucose stimulated insulin secretion (GSIS) assays to measure glucose responsiveness, insulin content and proinsulin-to-insulin ratio to determine intracellular insulin levels and processing, flow cytometry to measure the percentages of the different hormone-producing cells, and qRT-PCR and immunostaining to confirm the presence of β-cell-specific markers. In particular, SC-β cells can be identified by their coexpression of C-peptide and NKX6-1, while chromogranin A (CHGA) marks the general endocrine population. After aggregation, >80% of the cells in these clusters should express CHGA, with ˜20-60% of these cells being C-peptide+/NKX6-1+.

In certain embodiments, the SC-β cells achieve both first and second-phase dynamic insulin secretion. In some embodiments, the SC-β cells achieve equivalent functional capabilities of human islets. In certain embodiments, the SC-β cells retain functionality for 1 or more days. In certain embodiments, the SC-μμ cells retain functionality for more than 1 week. In certain embodiments, the SC-β cells may be used to treat or reverse severe preexisting diabetes at a rate similar to primary human islets, outperforming cells generated with a suspension-based protocol. In certain embodiments, the SC-β cells, when transplanted, are capable of maintaining normoglycemia indefinitely.

### 1. Assays to Assess SC-β Cell Function

A key functional feature of a β cell is its ability to repeatedly perform glucose stimulated insulin secretion (GSIS). In certain embodiments, assays can be performed to determine the physiological function in vitro of secreting insulin in response to glucose. In certain embodiments, the GSIS assay may be a perifusion GSIS (dynamic GSIS) assay (for example as in Velazco-Cruz, Stem Cell Reports, 2019).

In certain embodiments, other assays can be performed to examine the expression of specific genes, pathways, and transcription factors. Such assays include those detecting the presence of Yap (Rosado-Olivieri et al., 2019), the ROCKII pathway (Ghazizadeh et al., 2017), the transforming growth factor b (TGF-b) pathway (Velazco-Cruz, et al., 2019), the cytoskeleton (Hogrebe et al., 2020), and the expression of SIX2 (Velazco-Cruz et al., Cell Reports, 2020). In certain embodiments, other transcription factors important for the SC-β cell phenotype include PDX1, NKX6-1, NKX2-2, and NEUROD1 (Hogrebe et al., 2020).

In certain embodiments, an assay measuring changes in intracellular Ca2+ may be performed as described in Pagliuca et al. (Cell, 2014). β cells sense changing glucose levels through calcium signaling; increasing glucose levels leads to membrane depolarization causing an influx of calcium ions which triggers insulin exocytosis (Mohammed et al., 2009). In certain embodiments, the functional SC-β cells exhibit calcium flux similarly to primary human islet cells.

In certain embodiments, assays can also be performed to assess in vivo functionality of the SC-β cells. An example of such an assay can be found in Pagliuca et al. (Cell, 2014). Briefly, to test their capacity to function in vivo, SC-β cells are transplanted under the kidney capsule of immunocompromised mice and the ability of the cells to produce insulin is analyzed.

### D. Exemplary Embodiments of Modified Cells

In some embodiments, the cells and populations thereof exhibit increased expression of CD47 and reduced expression of one or more molecules of the MHC class I complex. In some embodiments, the cells and populations thereof exhibit increased expression of CD47 and reduced expression of one or more molecules of the MHC class I and MHC class II complexes. In some embodiments, the modified cells express one or more exogenous complement inhibitor polypeptides selected from CD46, CD59, CD55, and any combinations thereof. In some embodiments, the modified cells exhibit reduced expression of CD142.

In some embodiments, the cells and populations thereof exhibit increased expression of CD47 and reduced expression of B2M. In some embodiments, the cells and populations thereof exhibit increased expression of CD47 and reduced expression of CIITA. In some embodiments, the cells and populations thereof exhibit increased expression of CD47 and reduced expression of NLRC5. In some embodiments, the cells and populations thereof exhibit increased expression of CD47 and reduced expression of one or more molecules of B2M and CIITA. In some embodiments, the cells and populations thereof exhibit increased expression of CD47 and reduced expression of one or more molecules of B2M and NLRC5. In some embodiments, the cells and populations thereof exhibit increased expression of CD47 and reduced expression of one or more molecules of B2M, CIITA and NLRC5. Any of the cells described herein can also exhibit increased expression of one or more factors selected from the group including, but not limited to, DUX4, CD24, CD27, CD200, HLA-C, HLA-E, HLA-E heavy chain, HLA-G, PD-L1, IDO1, CTLA4-Ig, Cl-Inhibitor, IL-10, IL-35, IL-39, FasL, CCL21, CCL22, Mfge8, and Serpinb9. In some embodiments, the modified cells express one or more exogenous complement inhibitor polypeptides selected from CD46, CD59, CD55, and any combinations thereof.

In some embodiments, the cells and populations thereof exhibit increased expression of CD47, reduced expression of CD142, and reduced expression of B2M. In some embodiments, the cells and populations thereof exhibit increased expression of CD47, reduced expression of CD142, and reduced expression of CIITA. In some embodiments, the cells and populations thereof exhibit increased expression of CD47, reduced expression of CD142, and reduced expression of NLRC5. In some embodiments, the cells and populations thereof exhibit increased expression of CD47, reduced expression of CD142, and reduced expression of one or more molecules of B2M and CIITA. In some embodiments, the cells and populations thereof exhibit increased expression of CD47, reduced expression of CD142, and reduced expression of one or more molecules of B2M and NLRC5. In some embodiments, the cells and populations thereof exhibit increased expression of CD47, reduced expression of CD142, and reduced expression of one or more molecules of B2M, CIITA and NLRC5. Any of the cells described herein can also exhibit increased expression of one or more factors selected from the group including, but not limited to, DUX4, CD24, CD27, CD200, HLA-C, HLA-E, HLA-E heavy chain, HLA-G, PD-L1, IDO1, CTLA4-Ig, Cl-Inhibitor, IL-10, IL-35, IL-39, FasL, CCL21, CCL22, Mfge8, and Serpinb9. In some embodiments, the modified cells express one or more exogenous complement inhibitor polypeptides selected from CD46, CD59, CD55, and any combinations thereof.

In some embodiments, the cells and populations thereof exhibit increased expression of CD47 and at least one complement inhibitor selected from the group consisting of CD46, CD59, CD55, and any combination thereof, and reduced expression of one or more molecules of the MHC class I complex. In some embodiments, the cells and populations thereof exhibit increased expression of CD47 and at least one complement inhibitor selected from the group consisting of CD46, CD59, CD55, and any combination thereof, and reduced expression of one or more molecules of the MHC class II complex. In some embodiments, the cells and populations thereof exhibit increased expression of CD47 and at least one complement inhibitor selected from the group consisting of CD46, CD59, CD55, and any combination thereof, and reduced expression of one or more molecules of the MHC class II and MHC class II complexes. In some embodiments, the cells and populations thereof exhibit increased expression of CD47 and at least one complement inhibitor selected from the group consisting of CD46, CD59, CD55, and any combination thereof, and reduced expression of B2M. In some embodiments, the cells and populations thereof exhibit increased expression of CD47 and at least one complement inhibitor selected from the group consisting of CD46, CD59, CD55, and any combination thereof, and reduced expression of CIITA. In some embodiments, the cells and populations thereof exhibit increased expression of CD47 and at least one complement inhibitor selected from the group consisting of CD46, CD59, CD55, and any combination thereof, and reduced expression of NLRC5. In some embodiments, the cells and populations thereof exhibit increased expression of CD47 and at least one complement inhibitor selected from the group consisting of CD46, CD59, CD55, and any combination thereof, and reduced expression of one or more molecules of B2M and CIITA. In some embodiments, the cells and populations thereof exhibit increased expression of CD47 and at least one complement inhibitor selected from the group consisting of CD46, CD59, CD55, and any combination thereof, and reduced expression of one or more molecules of B2M and NLRC5. In some embodiments, the cells and populations thereof exhibit increased expression of CD47 and at least one complement inhibitor selected from the group consisting of CD46, CD59, CD55, and any combination thereof, and reduced expression of one or more molecules of CIITA and NLRC5. In some embodiments, the cells and populations thereof exhibit increased expression of CD47 at least one complement inhibitor selected from the group consisting of CD46, CD59, CD55, and any combination thereof, and reduced expression of one or more molecules of B2M, CIITA and NLRC5.

In some embodiments, the cells and populations thereof exhibit increased expression of CD47 and at least one complement inhibitor selected from the group consisting of CD46, CD59, CD55, and any combination thereof, reduced expression of CD142, and reduced expression of one or more molecules of the MHC class I complex. In some embodiments, the cells and populations thereof exhibit increased expression of CD47 and at least one complement inhibitor selected from the group consisting of CD46, CD59, CD55, and any combination thereof, reduced expression of CD142, and reduced expression of one or more molecules of the MHC class II complex. In some embodiments, the cells and populations thereof exhibit increased expression of CD47 and at least one complement inhibitor selected from the group consisting of CD46, CD59, CD55, and any combination thereof, reduced expression of CD142, and reduced expression of one or more molecules of the MHC class II and MHC class II complexes. In some embodiments, the cells and populations thereof exhibit increased expression of CD47 and at least one complement inhibitor selected from the group consisting of CD46, CD59, CD55, and any combination thereof, reduced expression of CD142, and reduced expression of B2M. In some embodiments, the cells and populations thereof exhibit increased expression of CD47 and at least one complement inhibitor selected from the group consisting of CD46, CD59, CD55, and any combination thereof, reduced expression of CD142, and reduced expression of CIITA. In some embodiments, the cells and populations thereof exhibit increased expression of CD47 and at least one complement inhibitor selected from the group consisting of CD46, CD59, CD55, and any combination thereof, reduced expression of CD142, and reduced expression of NLRC5. In some embodiments, the cells and populations thereof exhibit increased expression of CD47 and at least one complement inhibitor selected from the group consisting of CD46, CD59, CD55, and any combination thereof, reduced expression of CD142, and reduced expression of one or more molecules of B2M and CIITA. In some embodiments, the cells and populations thereof exhibit increased expression of CD47 and at least one complement inhibitor selected from the group consisting of CD46, CD59, CD55, and any combination thereof, reduced expression of CD142, and reduced expression of one or more molecules of B2M and NLRC5. In some embodiments, the cells and populations thereof exhibit increased expression of CD47 and at least one complement inhibitor selected from the group consisting of CD46, CD59, CD55, and any combination thereof, reduced expression of CD142, and reduced expression of one or more molecules of CIITA and NLRC5. In some embodiments, the cells and populations thereof exhibit increased expression of CD47 at least one complement inhibitor selected from the group consisting of CD46, CD59, CD55, and any combination thereof, reduced expression of CD142, and reduced expression of one or more molecules of B2M, CIITA and NLRC5.

In some embodiments, the modified SC-beta cells are differentiated from modified pluripotent stem cells that have been engineered to have reduced or increased expression of one or more targets relative to an unaltered or unmodified wild-type cell. In some embodiments, the unaltered or unmodified wild-type cell is the wild-type cell or the control cell that is the starting material for generating the modified pluripotent stem cells, or is a cell derived or differentiated from such cell. For instance, in some embodiments, an iPSC cell line starting material is a starting material that is considered a wild-type or control cell as contemplated herein. Hence, in some cases, the unaltered or unmodified cell may be an unmodified iPSC or progeny thereof that may contain nucleic acid changes resulting in pluripotency but did not undergo the gene editing procedures of the present disclosure, such as to achieve reduced expression of MHC I and/or II, and/or overexpression of CD47 proteins. However, by way of example, an unmodified iPSC, such as a “wild-type” or “control.” can also mean a starting material for generating the modified pluripotent stem cells that is an engineered cell that may contain nucleic acid changes resulting in reduced expression of MHC I and/or II, but did not undergo the gene editing procedures to result in overexpression of CD47 proteins. In some cases, the progeny of any such unmodified iPSC may be an unmodified SC-beta cell differentiated therefrom.

In some embodiments, a modified SC-beta cell exhibits increased or decreased expression of the one or more target molecules (e.g. MHC class I or class II, or CD47) in which the increase or decrease in expression is retained or similar (e.g. 75% to 100% of the level) compared to the unmodified or wild-type cell, such as an unmodified iPSC or unmodified SC-beta cell differentiated thereof. Also provided herein is a population of modified SC-beta cells that include a plurality of cells that exhibit increased or decreased expression of the one or more target molecules (e.g. MHC class I or class II, or CD47). In some embodiments, among a population of differentiated modified SC-beta cells at least 60% (e.g. at least 70%, at least 80%, at least 90% or more of the cells) retain or exhibit similar levels of the one or more target molecules (e.g. MHC class I or class II, or CD47) compared to the starting or modified pluripotent stem cells.

In some embodiments, at least at or about 50%, at least at or about 60%, at least at or about 70%, at least at or about 80%, or at least at or about 90% of the cells in the population are eliminated for expression of MHC class I or for B2M. In some embodiments, at least at or about 50%, at least at or about 60%, at least at or about 70%, at least at or about 80%, or at least at or about 90% of the cells in the population are eliminated for expression of MHC class II or for CIITA.

In some embodiments, least at or about 50%, at least at or about 60%, at least at or about 70%, at least at or about 80%, or at least at or about 90% of the cells in the population level have increased expression of the tolerogenic factor (CD47) that is greater than at or about 5-fold, greater than at or about 10-fold, greater than at or about 20-fold, greater than at or about 30-fold, greater than at or about 40-fold, greater than at or about 50-fold, greater than at or about 60-fold, or greater than at or about 70-fold over a wild-type primary beta cell or an unmodified pluripotent stem cell or an unmodified SC-beta differentiated from the unmodified pluripotent stem cell. In some embodiments, at least at or about 50%, at least at or about 60%, at least at or about 70%, at least at or about 80%, or at least at or about 90% of the cells in the population expresses the tolerogenic factor (e.g. CD47) at greater than at or about 20,000 molecules per cell, at greater than at or about 30,000 molecules per cell, greater than at or about 50,000 molecules per cell, greater than at or about 100,000 molecules per cell, greater than at or about 200,000 molecules per cell, greater than at or about 300,000 molecules per cell, greater than at or about 400,000 molecules per cell, greater than at or about 500,000 molecules per cell, or greater than at or about 600,000 molecules per cell.

In some embodiments, at least at or about 50%, at least at or about 60%, at least at or about 70%, at least at or about 80%, or at least at or about 90% of the cells in the population are eliminated for expression of CD142.

One skilled in the art will appreciate that levels of expression such as increased or reduced expression of a gene, protein or molecule can be referenced or compared to a comparable cell. In some embodiments, a modified cell (e.g., a modified SC-beta cell, such as a modified beta islet cell) having increased expression of a protein (e.g., CD46, CD59. CD55, CD47, or any other tolerogenic factor) refers to a modified cell (e.g., a modified beta islet cell) having a higher level of the protein compared to an unmodified cell. In some embodiments, a modified cell (e.g., a modified beta islet cell) having increased expression of a protein (e.g., CD46, CD59. CD55, CD47, or any other tolerogenic factor) is a cell (e.g., a modified beta islet cell) comprising modifications, wherein the cell comprising modifications has a higher level of the protein compared to a cell without said modifications (e.g., the stem cell without the modifications may comprise other modifications). In some embodiments, a modified cell (e.g., a modified beta islet cell) having reduced expression of a protein (e.g., CD142, B2M, or CIITA) is a cell comprising modifications, wherein the cell comprising modifications has a lower level of the protein or RNA compared to a cell without said modifications (e.g., the stem cell without the modifications may comprise other modifications). In some embodiments, the modified cells express one or more exogenous complement inhibitor polypeptides selected from CD46, CD59, CD55, and any combinations thereof.

In one embodiment, provided herein are modified cells (e.g., modified beta islet cells) expressing exogenous CD47 polypeptides and having reduced expression of CD142 and reduced expression of either one or more MHC class I complex proteins, one or more MHC class II complex proteins, or any combination of MHC class I and class II complex proteins. In another embodiment, the cells express exogenous CD47 polypeptides and express reduced levels CD142 of B2M and CIITA polypeptides. In some embodiments, the cells express exogenous CD47 polypeptides and possess modifications of the CD142, B2M and CIITA genes. In some instances, the modifications inactivate the B2M and CIITA genes. In some embodiments, the modified cells express one or more exogenous complement inhibitor polypeptides selected from CD46, CD59, CD55, and any combinations thereof.

In some embodiments, the cells (e.g., modified beta islet cells or other cell types that come into contact with the blood during transplantation) and populations thereof exhibit increased expression of CD47, and reduced expression of one or more molecules of the MHC class I complex, and are administered in combination with an anti-coagulant agent (e.g., heparin, melagatran, LMW-DS, N-acetylcysteine, alpha-1 antitrypsin (AAT) and/or activated protein C). In some embodiments, the cells and populations thereof exhibit increased expression of CD47 and reduced expression of one or more molecules of the MHC class I and MHC class II complexes, and are administered in combination with an anti-coagulant agent (e.g., heparin, melagatran, LMW-DS, N-acetylcysteine, alpha-1 antitrypsin (AAT) and/or activated protein C). In some embodiments, the modified cells express one or more exogenous complement inhibitor polypeptides selected from CD46, CD59, CD55, and any combinations thereof.

In some embodiments, the cells and populations thereof exhibit increased expression of CD47 and reduced expression of B2M, and are administered in combination with an anti-coagulant agent (e.g., heparin, melagatran, LMW-DS, N-acetylcysteine, alpha-1 antitrypsin (AAT) and/or activated protein C). In some embodiments, the cells and populations thereof exhibit increased expression of CD47 and reduced expression of CIITA, and are administered in combination with an anti-coagulant agent (e.g., heparin, melagatran, LMW-DS, N-acetylcysteine, alpha-1 antitrypsin (AAT) and/or activated protein C). In some embodiments, the cells and populations thereof exhibit increased expression of CD47 and reduced expression of NLRC5, and are administered in combination with an anti-coagulant agent (e.g., heparin, melagatran, LMW-DS, N-acetylcysteine, alpha-1 antitrypsin (AAT) and/or activated protein C). In some embodiments, the cells and populations thereof exhibit increased expression of CD47 and reduced expression of one or more molecules of B2M and CIITA, and are administered in combination with an anti-coagulant agent (e.g., heparin, melagatran, LMW-DS, N-acetylcysteine, alpha-1 antitrypsin (AAT) and/or activated protein C). In some embodiments, the cells and populations thereof exhibit increased expression of CD47 and reduced expression of one or more molecules of B2M and NLRC5, and are administered in combination with an anti-coagulant agent (e.g., heparin, melagatran, LMW-DS, N-acetylcysteine, alpha-1 antitrypsin (AAT) and/or activated protein C). In some embodiments, the cells and populations thereof exhibit increased expression of CD47 and reduced expression of one or more molecules of B2M, CIITA and NLRC5, and are administered in combination with an anti-coagulant agent (e.g., heparin, melagatran, LMW-DS, N-acetylcysteine, alpha-1 antitrypsin (AAT) and/or activated protein C). Any of the cells described herein can also exhibit increased expression of one or more factors selected from the group including, but not limited to, DUX4, CD24, CD27, CD46, CD55, CD59, CD200, HLA-C, HLA-E, HLA-E heavy chain, HLA-G, PD-L1, IDO1, CTLA4-Ig, Cl-Inhibitor, IL-10, IL-35, IL-39, FasL, CCL21, CCL22, Mfge8, and Serpinb9. In some embodiments, the modified cells express one or more exogenous complement inhibitor polypeptides selected from CD46, CD59, CD55, and any combinations thereof.

In some embodiments, the cells and populations thereof exhibit increased expression of CD47 and at least one complement inhibitor selected from the group consisting of CD46, CD59, CD55, and any combination thereof, and reduced expression of one or more molecules of the MHC class I complex, and are administered in combination with an anti-coagulant agent (e.g., heparin, melagatran, LMW-DS, N-acetylcysteine, alpha-1 antitrypsin (AAT) and/or activated protein C). In some embodiments, the cells and populations thereof exhibit increased expression of CD47 and at least one complement inhibitor selected from the group consisting of CD46, CD59, CD55, and any combination thereof, and reduced expression of one or more molecules of the MHC class II complex, and are administered in combination with an anti-coagulant agent (e.g., heparin, melagatran, LMW-DS, N-acetylcysteine, alpha-1 antitrypsin (AAT) and/or activated protein C). In some embodiments, the cells and populations thereof exhibit increased expression of CD47 and at least one complement inhibitor selected from the group consisting of CD46, CD59, CD55, and any combination thereof and reduced expression of one or more molecules of the MHC class II and MHC class II complexes, and are administered in combination with an anti-coagulant agent (e.g., heparin, melagatran, LMW-DS, N-acetylcysteine, alpha-1 antitrypsin (AAT) and/or activated protein C). In some embodiments, the cells and populations thereof exhibit increased expression of CD47 and at least one complement inhibitor selected from the group consisting of CD46, CD59, CD55, and any combination thereof and reduced expression of B2M, and are administered in combination with an anti-coagulant agent (e.g., heparin, melagatran, LMW-DS, N-acetylcysteine, alpha-1 antitrypsin (AAT) and/or activated protein C). In some embodiments, the cells and populations thereof exhibit increased expression of CD47 and at least one complement inhibitor selected from the group consisting of CD46, CD59, CD55, and any combination thereof and reduced expression of CIITA, and are administered in combination with an anti-coagulant agent (e.g., heparin, melagatran, LMW-DS, N-acetylcysteine, alpha-1 antitrypsin (AAT) and/or activated protein C). In some embodiments, the cells and populations thereof exhibit increased expression of CD47 and at least one complement inhibitor selected from the group consisting of CD46, CD59, CD55, and any combination thereof, and reduced expression of one or more molecules of B2M and CIITA, and are administered in combination with an anti-coagulant agent (e.g., heparin, melagatran, LMW-DS, N-acetylcysteine, alpha-1 antitrypsin (AAT) and/or activated protein C).

In one embodiment, provided herein are modified cells (e.g., modified beta islet cells) expressing exogenous CD47 polypeptides and having reduced expression of either one or more MHC class I complex proteins, one or more MHC class II complex proteins, or any combination of MHC class I and class II complex proteins. In another embodiment, the cells express exogenous CD47 polypeptides and express reduced levels of B2M and CIITA polypeptides. In some embodiments, the cells express exogenous CD47 polypeptides and possess modifications of the B2M and CIITA genes. In some instances, the modifications inactivate the B2M and CIITA genes. In some embodiments, the modified cells express one or more exogenous complement inhibitor polypeptides selected from CD46, CD59, CD55, and any combinations thereof. In some embodiments, the modified cells are administered to a patient in combination with an anti-coagulant agent (e.g., heparin, melagatran, LMW-DS, N-acetylcysteine, alpha-1 antitrypsin (AAT) and/or activated protein C).

### E. Assays for Hypoimmunogenic Phenotypes

In some embodiments, the provided modified cells are modified such that they are able to evade immune recognition and responses when administered to a patient (e.g., recipient subject). The cells can evade killing by immune cells in vitro and in vivo. In some embodiments, the cells evade killing by macrophages and NK cells. In some embodiments, the cells are ignored by immune cells or a subject's immune system. In other words, the cells administered in accordance with the methods described herein are not detectable by immune cells of the immune system. In some embodiments, the cells are cloaked and therefore avoid immune rejection.

Methods of determining whether a modified cell provided herein evades immune recognition include, but are not limited to, IFN-γ Elispot assays, microglia killing assays, cell engraftment animal models, cytokine release assays, ELISAs, killing assays using bioluminescence imaging or chromium release assay or Xcelligence analysis, mixed-lymphocyte reactions, immunofluorescence analysis, etc.

In some embodiments, the immunogenicity of the cells is evaluated in a complement-dependent cytotoxicity (CDC) assay. CDC can be assayed in vitro by incubating cells with IgG or IgM antibodies targeting an HLA-independent antigen expressed on the cell surface in the presence of serum containing complement and analyzing cell killing. In some embodiments, CDC can be assayed by incubating cells with ABO blood type incompatible serum, wherein the cells comprise A antigens or B antigens, and the serum comprises antibodies against the A antigens and/or B antigens of the cells.

In some embodiments, once the modified cells have been modified or generated as described herein, they may be assayed for their hypoimmunogenicity. Any of a variety of assays can be used to assess if the cells are hypoimmunogenic or can evade the immune system. Exemplary assays include any as is described in WO2016183041 and WO2018132783.

In some embodiments, the modified cells described herein survive in a host without stimulating the host immune response for one week or more (e.g., one week, two weeks, one month, two months, three months, 6 months, one year, two years, three years, four years, five years or more, e.g., for the life of the cell and/or its progeny). The cells maintain expression of the transgenes and/or are deleted or reduced in expression of target genes for as long as they survive in the host. In some aspects, if the transgenes are no longer expressed and/or if target genes are expressed the modified cells may be removed by the host's immune system. In some embodiments, the persistence or survival of the modified cells may be monitored after their administration to a recipient by further expressing a transgene encoding a protein that allows the cells to be detected in vivo (e.g., a fluorescent protein, such as GFP, a truncated receptor or other surrogate marker or other detectable marker).

The hypoimmunogenic cells are administered in a manner that permits them to engraft to the intended tissue site and reconstitute or regenerate the functionally deficient area. In some embodiments, the hypoimmunogenic cells are assayed for engraftment (e.g., successful engraftment). In some embodiments, the engraftment of the hypoimmunogenic cells is evaluated after a pre-selected amount of time. In some embodiments, the engrafted cells are monitored for cell survival. For example, the cell survival may be monitored via bioluminescence imaging (BLI), wherein the cells are transduced with a luciferase expression construct for monitoring cell survival. In some embodiments, the engrafted cells are visualized by immunostaining and imaging methods known in the art. In some embodiments, the engrafted cells express known biomarkers that may be detected to determine successful engraftment. For example, flow cytometry may be used to determine the surface expression of particular biomarkers. In some embodiments, the hypoimmunogenic cells are engrafted to the intended tissue site as expected (e.g., successful engraftment of the hypoimmunogenic cells). In some embodiments, the hypoimmunogenic cells are engrafted to the intended tissue site as needed, such as at a site of cellular deficiency. In some embodiments, the hypoimmunogenic cells are engrafted to the intended tissue site in the same manner as a cell of the same type not comprising the modifications.

In some embodiments, administering the populations of modified cells (e.g., modified beta islet cells comprising modifications including reduced CD142 expression) or combinations (e.g., administering a population of modified cells in combination with an anti-coagulant agent) improves survival and engraftment by allowing cells to avoid or reduce IBMIR that occurs as a result of exposure of the cells to blood during transplant. In some embodiments, the reduction in IBMIR reduces the amount of cell loss (e.g., loss of transplanted islets) that occurs during transplant.

In some embodiments, the hypoimmunogenic cells are assayed for function. In some embodiments, the hypoimmunogenic cells are assayed for function prior to their engraftment to the intended tissue site. In some embodiments, the hypoimmunogenic cells are assayed for function following engraftment to the intended tissue site. In some embodiments, the function of the hypoimmunogenic cells is evaluated after a pre-selected amount. In some embodiments, the function of the engrafted cells is evaluated by the ability of the cells to produce a detectable phenotype. For example, engrafted beta islet cells function may be evaluated based on the restoration of lost glucose control due to diabetes. In some embodiments, the function of the hypoimmunogenic cells is as expected (e.g., successful function of the hypoimmunogenic cells while avoiding antibody-mediated rejection). In some embodiments, the function of the hypoimmunogenic cells is as needed, such as sufficient function at a site of cellular deficiency while avoiding antibody-mediated rejection. In some embodiments, the modified cells function in the same manner as a non-modified cell of the same type.

### 1. Instant Blood-Mediated Inflammatory Reaction

In some embodiments, the modified cells provided herein evade an instant blood-mediated inflammatory reaction. A major contributor to the poor outcome of clinical islet transplantation is the occurrence of the destructive instant blood mediated inflammatory reaction (IBMIR), which leads to loss of transplanted tissue when the islets encounter the blood in the portal vein (Bennet et al., (1995) Diabetes 48:1907-1914; Moberg et al., (2002) Lancet 360:2039-2045). This reaction is triggered by tissue factor (TF) expression by the endocrine cells of the islets, combined with an array of other proinflammatory events, such as the expression of MCP-1 (Piemonti et al., (2002) Diabetes 51:55-65), IL-8, and MIF (Waeber et al., (1997) Proc Natl Acad Sci USA 94:4782-4787; Johansson et al., (2006) Am J Transplantation 6 (2): 305).

Instant blood-mediated inflammatory reaction (IBMIR) is a nonspecific inflammatory and thrombotic reaction that can occur when cells expressing CD142 come into contact with blood. IBMIR is initiated rapidly by exposure to human blood in the portal vein. It is characterized by activation of complement, platelets, and the coagulation pathway, which in turn leads to the recruitment of neutrophils. IBMIR causes significant loss of transplanted islets. In some embodiments, provided herein are compositions (e.g., modified cells comprising reduced expression of CD142 in combination with one or more of the other modifications described herein), combinations (e.g., a combination comprising any of the populations of modified cells described herein and an anti-coagulant agent that reduces coagulation), and methods (e.g., methods of treating a patient comprising administering any of the populations of modified cells described herein and anti-coagulant agent that reduces coagulation) that reduce an IBMIR associated with transplantation of the cells or exposure of the cells to blood.

In some embodiments, IBMIR can be assayed in vitro, for example, in an in vitro tubing loop model of IBMIR, which has been previously described in U.S. Pat. No. 7,045,502, which is herein incorporated by reference in its entirety.

In some embodiments, IBMIR can be assayed in vivo (e.g., in a mammal or in a human patient) by drawing blood samples during the peritransplant period and evaluating plasma levels of thrombin-anti-thrombin III complex (TAT), C-peptide, factor XIa-antithrombin (FXIa-AT), factor XIIa-antithrombin (FXIIa-AT), thrombin-antithrombin (TAT) plasmin-alpha 2 antiplasmin (PAP), and/or complement C3a. sIn some embodiments, IBMIR is associated with increased levels of TAT, C-peptide, FXIa-AT, FXIIa-AT, PAP, and/or complement C3a during infusion of transplanted cells and/or in a period of time following transplant (e.g., up to 3, 5, 10, or more than 10 hours after transplant). In some embodiments, IBMIR can be assayed by monitoring counts of free circulating platelets, wherein a decrease in the counts of platelets during or following transplantation is associated with IBMIR (e.g., with platelet consumption due to IBMIR).

### 2. Complement Dependent Cytotoxicity

In some embodiments, the modified cells (e.g., beta islets) provided herein evade complement dependent cytotoxicity (CDC). In some embodiments, the CDC is secondary to a thrombotic reaction of IBMIR. In some embodiments, the CDC occurs independently of IBMIR.

In some embodiments, susceptibility of cells to CDC can be analyzed in vitro according to standard protocols understood by one of ordinary skill in the art. In some embodiments, CDC can be analyzed in vitro by mixing serum comprising the components of the complement system (e.g., human serum), with target cells bound by an antibody (e.g., an IgG or IgM antibody), and then to determine cell death. In some embodiments, susceptibility of cells to CDC can be analyzed in vitro by incubating cells in the presence of ABO-incompatible or Rh factor incompatible serum, comprising the components of the complement system and antibodies against ABO type A, ABO type B, and/or Rh factor antigens of the cells.

A common CDC assay determines cell death via pre-loading the target cells with a radioactive compound. As cells die, the radioactive compound is released from them. Hence, the efficacy of the antibody to mediate cell death is determined by the radioactivity level. Unlike radioactive CDC assays, non-radioactive CDC assays often determine the release of abundant cell components, such as GAPDH, with fluorescent or luminescent determination. In some embodiments, cell killing by CDC can be analyzed using a label-free platform such as xCELLigence™ (Agilent).

### III. Populations of Modified Cells and Pharmaceutical Compositions

Provided herein are populations modified cells containing a plurality of the provided modified cells, such as modified SC-beta cells.

In some embodiments, the population of modified SC-beta cells are derived from modified pluripotent cells, such as modified iPSCs. In some embodiments, at least 50%, 60%, 70%, 80%, 90%, 95%, 96%, 97%, 98%, 99%, 99.9%, or 99.99% of cells in the population of starting modified pluripotent stem cells comprise the modifications. In some embodiments, at least 50%, 60%, 70%, 80%, 90%, 95%, 96%, 97%, 98%, 99%, 99.9%, or 99.99% of SC-beta cells differentiated from the starting modified pluripotent stem cells comprise the modifications.

In some embodiments, at least about any of 50%, 60%, 70%, 80%, 90%, 95%, 96%, 97%, 98%, 99%, 99.9%, or 99.99% of cells in a population of SC-beta cells differentiated from modified pluripotent stem cells comprise reduced expression of MHC class I molecule and/or MHC class II molecule relative to an unmodified or unaltered cell pluripotent stem cell that does not comprise the one or more modifications, or an SC-beta cell differentiated from the unmodified pluripotent stem cell. In some embodiments, at least about any of 50%, 60%, 70%, 80%, 90%, 95%, 96%, 97%, 98%, 99%, 99.9%, or 99.99% of cells in a population of SC-beta cells differentiated from modified pluripotent stem cells comprise reduced expression of B2M and/or CIITA relative to an unmodified or unaltered cell of the same cell type that does not comprise the one or more modifications, or an SC-beta cell differentiated from the unmodified pluripotent stem cell. In some embodiments, at least about any of 50%, 60%, 70%, 80%, 90%, 95%, 96%, 97%, 98%, 99%, 99.9%, or 99.99% of cells in a population of SC-beta cells differentiated from modified pluripotent stem cells comprise reduced expression of B2M and CIITA relative to an unmodified or unaltered cell of the same cell type that does not comprise the one or more modifications, or an SC-beta cell differentiated from the unmodified pluripotent stem cell. In some embodiments, at least about any of 50%, 60%, 70%, 80%, 90%, 95%, 96%, 97%, 98%, 99%, 99.9%, or 99.99% of cells in a population of SC-beta cells differentiated from unmodified pluripotent stem cells comprise one or more alterations that inactivate both alleles of a B2M gene relative to an unmodified or unaltered cell of the same cell type that does not comprise the one or more modifications, or an SC-beta cell differentiated from the unmodified pluripotent stem cell. In some embodiments, at least about any of 50%, 60%, 70%, 80%, 90%, 95%, 96%, 97%, 98%, 99%, 99.9%, or 99.99% of cells in a population of SC-beta cells differentiated from modified pluripotent stem cells comprise one or more alterations that inactivate both alleles of a CIITA gene relative to an unmodified or unaltered cell of the same cell type that does not comprise the one or more modifications, or an SC-beta cell differentiated from the unmodified pluripotent stem cell.

In some embodiments, at least 50%, 60%, 70%, 80%, 90%, 95%, 96%, 97%, 98%, 99%, 99.9%, or 99.99% of cells in the population comprise one or more alterations that inactivate both alleles of an endogenous B2M gene. In some embodiments, at least 50%, 60%, 70%, 80%, 90%, 95%, 96%, 97%, 98%, 99%, 99.9%, or 99.99% of cells in the population comprise one or more alterations that inactivate both alleles of an endogenous CIITA gene.

In some embodiments at least 50%, 60%, 70%, 80%, 90%, 95%, 96%, 97%, 98%, 99%, 99.9%, or 99.99% of cells in a population of SC-beta cells differentiated from modified pluripotent stem cells comprise a set of modifications that reduce expression of MHC class I molecules and/or MHC class II molecules, that increase expression of one or more tolerogenic factors, and that reduce expression of CD142, relative to an unmodified or unaltered cell pluripotent stem cell that does not comprise the one or more modifications, or an SC-beta cell differentiated from the unmodified pluripotent stem cell. In some embodiments, the one or more tolerogenic factors is one or more of DUX4, B2M-HLA-E, CD16, CD52, CD47, CD27, CD200, HLA-C, HLA-E, HLA-E heavy chain, HLA-G, PD-L1, IDO1, CTLA4-Ig, Cl-Inhibitor, IL-10, IL-35, FASL, CCL21, MFGE8, SERPINB9, CD35, IL-39, CD16 Fc Receptor, IL15-RF, and H2-M3, or any combination thereof. In some embodiments, the one or more tolerogenic factors is CD47.

In some embodiments, at least 50%, 60%, 70%, 80%, 90%, 95%, 96%, 97%, 98%, 99%, 99.9%, or 99.99% of cells in a population of SC-beta cells differentiated from modified pluripotent stem cells comprise an exogenous polynucleotide encoding CD46, relative to an unmodified or unaltered cell pluripotent stem cell that does not comprise the one or more modifications, or an SC-beta cell differentiated from the unmodified pluripotent stem cell. In some embodiments, at least 50%, 60%, 70%, 80%, 90%, 95%, 96%, 97%, 98%, 99%, 99.9%, or 99.99% of cells in a population of SC-beta cells differentiated from modified pluripotent stem cells comprise an exogenous polynucleotide encoding CD59, relative to an unmodified or unaltered cell pluripotent stem cell that does not comprise the one or more modifications, or an SC-beta cell differentiated from the unmodified pluripotent stem cell. In some embodiments, at least 50%, 60%, 70%, 80%, 90%, 95%, 96%, 97%, 98%, 99%, 99.9%, or 99.99% of cells in a population of SC-beta cells differentiated from modified pluripotent stem cells comprise an exogenous polynucleotide encoding CD55, relative to an unmodified or unaltered cell pluripotent stem cell that does not comprise the one or more modifications, or an SC-beta cell differentiated from the unmodified pluripotent stem cell.

In some embodiments, at least 50%, 60%, 70%, 80%, 90%, 95%, 96%, 97%, 98%, 99%, 99.9%, or 99.99% of cells in the population express one or more markers of a beta cell or a marker of an islet. The marker may include one or more of INS, CHGA, NKX2-2, PAX6, PDX1, NKX6-1, MAFB, GCK and GLUT1.

Also provided herein are compositions comprising the modified cells or populations of modified cells. In some embodiments, the compositions are pharmaceutical compositions.

In some embodiments, the pharmaceutical composition provided herein further include a pharmaceutically acceptable excipient or carrier. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polysorbates (TWEEN™), poloxamers (PLURONICS™) or polyethylene glycol (PEG). In some embodiments, the pharmaceutical composition includes a pharmaceutically acceptable buffer (e.g., neutral buffer saline or phosphate buffered saline). In some embodiments, the pharmaceutical composition can contain one or more excipients for modifying, maintaining or preserving, for example, the pH, osmolarity, viscosity, clarity, color, isotonicity, odor, sterility, stability, rate of dissolution or release, adsorption, or penetration of the composition. In some aspects, a skilled artisan understands that a pharmaceutical composition containing cells may differ from a pharmaceutical composition containing a protein.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

The pharmaceutical composition in some embodiments contains modified SC-beta cells, such as iPSC-derived beta islet cells, as described herein in amounts effective to treat or prevent the disease or condition, such as a therapeutically effective or prophylactically effective amount. In some embodiments, the pharmaceutical composition contains modified cells as described herein in amounts effective to treat or prevent the disease or condition, such as a therapeutically effective or prophylactically effective amount. Therapeutic or prophylactic efficacy in some embodiments is monitored by periodic assessment of treated subjects. For repeated administrations over several days or longer, depending on the condition, the treatment is repeated until a desired suppression of disease symptoms occurs. However, other dosage regimens may be useful and can be determined. The desired dosage can be delivered by a single bolus administration of the composition, by multiple bolus administrations of the composition, or by continuous infusion administration of the composition.

In some embodiments, modified cells as described herein are administered using standard administration techniques, formulations, and/or devices. In some embodiments, modified cells as described herein are administered using standard administration techniques, formulations, and/or devices. Provided are formulations and devices, such as syringes and vials, for storage and administration of the compositions. Modified cells can be administered via localized injection, including catheter administration, systemic injection, localized injection, intravenous injection, or parenteral administration. When administering a therapeutic composition (e.g., a pharmaceutical composition containing a modified cell), it will generally be formulated in a unit dosage injectable form (solution, suspension, emulsion).

Formulations include those for intravenous, intraperitoneal, or subcutaneous, administration. In some embodiments, the cell populations are administered parenterally. The term “parenteral,” as used herein, includes intravenous, intramuscular, subcutaneous, rectal, vaginal, and intraperitoneal administration. In some embodiments, the cell populations are administered to a subject using peripheral systemic delivery by intravenous, intraperitoneal, or subcutaneous injection.

Compositions in some embodiments are provided as sterile liquid preparations, e.g., isotonic aqueous solutions, suspensions, emulsions, or dispersions, which may in some aspects be buffered to a selected pH. Liquid compositions are somewhat more convenient to administer, especially by injection. Liquid compositions can comprise carriers, which can be a solvent or dispersing medium containing, for example, water, saline, phosphate buffered saline, polyol (for example, glycerol, propylene glycol, liquid polyethylene glycol) and suitable mixtures thereof. Sterile injectable solutions can be prepared by incorporating the cells in a solvent, such as in admixture with a suitable carrier, diluent, or excipient such as sterile water, physiological saline, glucose, dextrose, or the like.

In some embodiments, a pharmaceutically acceptable carrier can include all solvents, dispersion media, coatings, antibacterial and antifungal agents, isotonic and absorption delaying agents, and the like, compatible with pharmaceutical administration (Gennaro, 2000, Remington: The science and practice of pharmacy, Lippincott, Williams & Wilkins, Philadelphia, PA). Examples of such carriers or diluents include, but are not limited to, water, saline, Ringer's solutions, dextrose solution, and 5% human serum albumin. Liposomes and non-aqueous vehicles such as fixed oils may also be used. Supplementary active compounds can also be incorporated into the compositions. The pharmaceutical carrier should be one that is suitable for the modified cells, such as a saline solution, a dextrose solution or a solution comprising human serum albumin. In some embodiments, the pharmaceutically acceptable carrier or vehicle for such compositions is any non-toxic aqueous solution in which the modified cells can be maintained, or remain viable, for a time sufficient to allow administration of live cells. For example, the pharmaceutically acceptable carrier or vehicle can be a saline solution or buffered saline solution.

In some embodiments, the composition, including pharmaceutical composition, is sterile. In some embodiments, isolation, enrichment, or culturing of the cells is carried out in a closed or sterile environment, for example and for instance in a sterile culture bag, to minimize error, user handling and/or contamination. In some embodiments, sterility may be readily accomplished, e.g., by filtration through sterile filtration membranes. In some embodiments, culturing is carried out using a gas permeable culture vessel. In some embodiments, culturing is carried out using a bioreactor.

Also provided herein are compositions that are suitable for cryopreserving the provided modified cells. In some embodiments, the provided modified cells are cryopreserved in a cryopreservation medium. In some embodiments, the cryopreservation medium is a serum free cryopreservation medium. In some embodiments, the composition comprises a cryoprotectant. In some embodiments, the cryoprotectant is or comprises DMSO and/or s glycerol. In some embodiments, the cryopreservation medium is between at or about 5% and at or about 10% DMSO (v/v). In some embodiments, the cryopreservation medium is at or about 5% DMSO (v/v). In some embodiments, the cryopreservation medium is at or about 6% DMSO (v/v). In some embodiments, the cryopreservation medium is at or about 7% DMSO (v/v). In some embodiments, the cryopreservation medium is at or about 7.5% DMSO (v/v). In some embodiments, the cryopreservation medium is at or about 8% DMSO (v/v). In some embodiments, the cryopreservation medium is at or about 9% DMSO (v/v). In some embodiments, the cryopreservation medium is at or about 10% DMSO (v/v). In some embodiments, the cryopreservation medium contains a commercially available cryopreservation solution (CryoStor™ CS10). CryoStor™ CS10 is a cryopreservation medium containing 10% dimethyl sulfoxide (DMSO). In some embodiments, compositions formulated for cryopreservation can be stored at low temperatures, such as ultra-low temperatures, for example, storage with temperature ranges from −40° C. to −150° C., such as or about 80° C.±6.0° C.

In some embodiments, the pharmaceutical composition comprises modified cells described herein and a pharmaceutically acceptable carrier comprising 31.25% (v/v) Plasma-Lyte A, 31.25% (v/v) of 5% dextrose/0.45% sodium chloride, 10% dextran 40 (LMD)/5% dextrose, 20% (v/v) of 25% human serum albumin (HSA), and 7.5% (v/v) dimethylsulfoxide (DMSO).

In some embodiments, the cryopreserved modified cells are prepared for administration by thawing. In some cases, the modified cells can be administered to a subject immediately after thawing. In such an embodiment, the composition is ready-to-use without any further processing. In other cases, the modified cells are further processed after thawing, such as by resuspension with a pharmaceutically acceptable carrier, incubation with an activating or stimulating agent, or are activated washed and resuspended in a pharmaceutically acceptable buffer prior to administration to a subject.

### IV. Methods of Treatment

Provided herein are compositions and methods relating to the provided cell compositions comprising a population of modified SC-beta cells (e.g. modified iPSC-derived beta islet cells) described herein for use in treating diseases or conditions in a subject. Provided herein is a method of treating a patient by administering a population modified SC-beta cells (e.g. modified iPSC-derived beta islet cells) described herein. In some embodiments, the population of cells are formulated for administration in a pharmaceutical composition, such as any described here. Such methods and uses include therapeutic methods and uses, for example, involving administration of the population of modified cells, or compositions containing the same, to a subject having a disease, condition, or disorder. It is within the level of a skilled artisan to choose the appropriate modified cells as provided herein for a particular disease indication. In some embodiments, the cells or pharmaceutical composition thereof is administered in an effective amount to effect treatment of the disease or disorder. Uses include uses of the modified cells or pharmaceutical compositions thereof in such methods and treatments, and in the preparation of a medicament in order to carry out such therapeutic methods. In some embodiments, the methods thereby treat the disease or condition or disorder in the subject.

The modified cells provided herein can be administered to any suitable patients including, for example, a candidate for a cellular therapy for the treatment of a disease or disorder. Candidates for cellular therapy include any patient having a disease or condition that may potentially benefit from the therapeutic effects of the subject modified cells provided herein. In some embodiments, the patient is an allogenic recipient of the administered cells. In some embodiments, the provided modified cells are effective for use in allogeneic cell therapy. A candidate who benefits from the therapeutic effects of the subject modified cells provided herein exhibit an elimination, reduction or amelioration of ta disease or condition.

In some embodiments, the cellular deficiency is associated with diabetes or the cellular therapy is for the treatment of diabetes. In some embodiments, the diabetes is Type I diabetes. In some embodiments, the diabetes is Type II diabetes. In some embodiments, the population of modified SC-beta cells is a population of iPSC-derived islet cells, including beta islet cells. In some embodiments, the method comprises administering to the patient a composition comprising a population of modified SC-beta cells (e.g. iPSC-derived beta islet cells), wherein the modified SC-b cells are modified as described herein. In some embodiments, the modified SC-b cells comprise: (i) a transgene comprising an exogenous polynucleotide encoding CD47, and (ii) inactivation or disruption of both alleles of a B2M gene and/or inactivation or disruption of both alleles of a CIITA gene. In some embodiments, the modified SC-b cells comprise: (i) a transgene comprising an exogenous polynucleotide encoding CD47, (ii) inactivation or disruption of both alleles of a B2M gene and (iii) inactivation or disruption of both alleles of a CIITA gene.

In certain embodiments the modified cells further have reduced CD142 expression. In some embodiments, the modified cells further comprise overexpression of one or more complement inhibitor. In some embodiments, the modified cells further comprise overexpression of one or more complement inhibitors selected from CD46, CD59, and CD55. In some embodiments, the modified cells comprise overexpression of CD46 and CD59. In some embodiments, the modified cells comprise overexpression of CD46, CD59, and CD59. In some embodiments, such modifications are useful when cells administered to the subject come into contact with the blood during or after administration. For instance, such modifications may prevent or attenuate an IBMIR when the modified cells come into contact with the blood. In some embodiments, the modified cells are administered intravenously or via intramuscular injection.

In some embodiments, the cellular deficiency is associated with diabetes or the cellular therapy is for the treatment of diabetes. In some embodiments, the diabetes is Type I diabetes. In some embodiments, the diabetes is Type II diabetes. In some embodiments, the population of modified cells is a population of islet cells, including beta islet cells. In some embodiments, the islet cells are selected from the group consisting of an islet progenitor cell, an immature islet cell, and a mature islet cell. In some embodiments, the method comprises administering to the patient a composition comprising a population of modified beta islet cells, wherein the modified beta islet cells comprise: (i) a transgene comprising an exogenous polynucleotide encoding CD47 and (ii) inactivation or disruption of both alleles of a B2M gene. In some embodiments, the method comprises administering to the patient a composition comprising a population of modified beta islet cells, wherein the modified beta islet cells comprise: (i) a transgene comprising an exogenous polynucleotide encoding CD47, (ii) inactivation or disruption of both alleles of a CD142 gene, and (iii) inactivation or disruption of both alleles of a B2M gene. In some embodiments, the method comprises administering to the patient a composition comprising a population of modified beta islet cells, wherein the modified beta islet cells comprise: (i) a transgene comprising an exogenous polynucleotide encoding CD47, and (ii) inactivation or disruption of both alleles of a B2M gene, wherein the method further comprises administering an anti-coagulant agent to the patient (e.g., heparin, or any of the anti-coagulant agents described herein). In some embodiments, the modified beta cells comprise inactivation or disruption of both alleles of a CIITA gene. In some embodiments, the transgene comprising the polynucleotide encoding CD47 is the transgene is a multicistronic vector, and the transgene further comprises an exogenous polynucleotide encoding CD46 and an exogenous polynucleotide encoding CD59. In other embodiments, the beta islet cells further comprise a separate multicistronic vector, wherein the multicistronic vector comprises an exogenous polynucleotide encoding CD46 and an exogenous polynucleotide encoding CD59.

In some aspects, the subject has, or has an increased risk of developing, a metabolic disorder. In some aspects, administering to the subject comprises implanting modified SC-beta cells or a population containing modified SC-beta cells. The subject may be a human subject or an animal subject. In some aspects, the cells may be implanted as dispersed cells or formed into clusters. In some embodiments, the cells or clusters may be infused into the hepatic portal vein.

In some embodiments, administration of the modified SC-beta cells (e.g. iPSC-derived beta islet cells) described herein may improve glucose tolerance in a subject. Glucose tolerance may be measured by any suitable method, such as those described herein (e.g., insulin secretion assays). In some embodiments, the engineered primary beta islet cell exhibits glucose-stimulated insulin secretion (GSIS).

Thus, in some embodiments, the improved glucose tolerance is measured in a GSIS perfusion assay. Glucose intolerance is related to insulin resistance, and can cause diabetes (e.g., Type 1 diabetes and Type II diabetes). Therefore, in some embodiments, provided is a method of treating diabetes comprising administering the provided modified SC-beta cells (e.g. iPSC-derived beta islet cells), or a composition comprising a population of modified SC-beta cells (e.g. iPSC-derived beta islet cells), to a subject in need thereof. In some embodiments, the subject is a diabetic patient. In some embodiments, the subject has Type I diabetes. In some embodiments, the subject has Type II diabetes. Specifically, in some embodiments, provided is a method of improving glucose tolerance in a subject, the method comprising administering a modified SC-beta cells (e.g. iPSC-derived beta islet cells), or a composition comprising a population of modified SC-beta cells (e.g. iPSC-derived beta islet cells), to a subject in need thereof. In some embodiments, glucose tolerance is improved relative to the subject's glucose tolerance prior to administration of the islet cells. In some embodiments, the beta cells reduce exogenous insulin usage in the subject. In some embodiments, glucose tolerance is improved as measured by HbA1c levels. In some embodiments, the subject is fasting. In some embodiments, the islet cells improve insulin secretion in the subject. In some embodiments, insulin secretion is improved relative to the subject's insulin secretion prior to administration of the islet cells.

In some embodiments, the modified SC-beta cells (e.g. iPSC-derived beta islet cells) may not induce and adaptive immune response in the subject. In some embodiments, the adaptive immune response is assessed using ELISPOT. For example, the adaptive immune response may be assessed by measuring the levels of IFNg cytokine secretion by CD8+ T cells. In some embodiments, the modified SC-beta cells (e.g. iPSC-derived beta islet cells) exhibit lower levels of IFNg compared to wild type primary beta islet cells or compared to SC-beta cells derived from unmodified pluripotent stem cells, such as any of about 400-fold, 300-fold, 200-fold, 100-fold, 50-fold, 25-fold, and 10-fold lower levels of IFNg compared to wild type primary beta islet cells or compared to SC-beta cells derived from unmodified pluripotent stem cells. In some embodiments, the adaptive immune response is assessed using flow cytometry. For example, in some embodiments, the adaptive immune response is assessed by measuring the levels donor specific antibody (DSA) IgG or IgM. In some embodiments, the modified SC-beta cells (e.g. iPSC-derived beta islet cells) exhibit lower levels of DSA levels compared to wild type primary beta islet cells, such as any of about 2-fold, 1.5-fold, and 1-fold lower levels of DSA compared to wild type primary beta islet cells or compared to SC-beta cells derived from unmodified pluripotent stem cells.

In some embodiments, the patient undergoing a treatment using the provided modified cells, or a composition containing the same, received a previous treatment. In some embodiments, the modified cells, or a composition containing the same, are used to treat the same condition as the previous treatment. In certain embodiments, the modified cells, or a composition containing the same, are used to treat a different condition from the previous treatment. In some embodiments, the modified cells, or a composition containing the same, administered to the patient exhibit an enhanced therapeutic effect for the treatment of the same condition or disease treated by the previous treatment. In certain embodiments, the administered modified cells, or a composition containing the same, exhibit a longer therapeutic effect for the treatment of the condition or disease in the patient as compared to the previous treatment. In exemplary embodiments, the administered cells exhibit an enhanced potency, efficacy and/or specificity against the cancer cells as compared to the previous treatment.

The methods provided herein can be used as a second-line treatment for a particular condition or disease after a failed first line treatment. In some embodiments, the previous treatment is a therapeutically ineffective treatment. As used herein, a “therapeutically ineffective” treatment refers to a treatment that produces a less than desired clinical outcome in a patient. For example, with respect to a treatment for a cellular deficiency, a therapeutically ineffective treatment may refer to a treatment that does not achieve a desired level of functional cells and/or cellular activity to replace the deficient cells in a patient, and/or lacks therapeutic durability. Therapeutic effectiveness can be measured using any suitable technique known in the art. In some embodiments, the patient produces an immune response to the previous treatment. In some embodiments, the previous treatment is a cell, tissue or organ graft that is rejected by the patient. In some embodiments, the previous treatment included a mechanically assisted treatment. In some embodiments, the mechanically assisted treatment included a hemodialysis or a ventricle assist device. In some embodiments, the patient produced an immune response to the mechanically assisted treatment. In some embodiments, the previous treatment included a population of therapeutic cells that include a safety switch that can cause the death of the therapeutic cells should they grow and divide in an undesired manner. In certain embodiments, the patient produces an immune response as a result of the safety switch induced death of therapeutic cells. In certain embodiments, the patient is sensitized from the previous treatment. In exemplary embodiments, the patient is not sensitized by the administered modified cells as provided herein.

In some embodiments, the provided modified cells, or compositions containing the same, are administered prior to providing a tissue, organ or partial organ transplant to a patient in need thereof. In particular embodiments, the patient does not exhibit an immune response to the modified cells. In certain embodiments, the modified cells are administered to the patient for the treatment of a cellular deficiency in a particular tissue or organ and the patient subsequently receives a tissue or organ transplant for the same particular tissue or organ. In such embodiments, the modified cell treatment functions as a bridge therapy to the eventual tissue or organ replacement. In certain embodiments, the modified cells are administered to the patient for the treatment of a cellular deficiency in a particular tissue or organ and the patient subsequently receives a tissue or organ transplant for a different tissue or organ. For example, in some embodiments, the patient is a diabetes patient who is treated with modified pancreatic beta cells as provided herein prior to receiving a kidney transplant. In some embodiments, the method is for the treatment of a cellular deficiency.

The methods of treating a patient are generally through administrations of modified cells, or a composition containing the same, as provided herein. As will be appreciated, for all the multiple embodiments described herein related to the cells and/or the timing of therapies, the administering of the cells is accomplished by a method or route that results in at least partial localization of the introduced cells at a desired site. The cells can be implanted directly to the desired site, or alternatively be administered by any appropriate route which results in delivery to a desired location in the subject where at least a portion of the implanted cells or components of the cells remain viable. In some embodiments, the cells are administered to treat a disease or disorder, such as any disease, disorder, condition, or symptom thereof that can be alleviated by cell therapy.

In some embodiments, the population of modified cells, or a composition containing the same, is administered at least 1 day, at least 2 days, at least 3 days, at least 4 days, at least 5, days, at least 6 days, at least 1 week, or at least 1 month or more after the patient is sensitized. In some embodiments, the population of modified cells, or a composition containing the same, is administered at least 1 week (e.g., 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, or more) or more after the patient is sensitized or exhibits characteristics or features of sensitization. In some embodiments, the population of modified cells, or a composition containing the same, is administered at least 1 month (e.g., 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, or more) or more after the patient has received the transplant (e.g., an allogeneic transplant), has been pregnant (e.g., having or having had alloimmunization in pregnancy) or is sensitized or exhibits characteristics or features of sensitization.

In some embodiments, the patient who has received a transplant, who has been pregnant (e.g., having or having had alloimmunization in pregnancy), and/or who is sensitized against an antigen (e.g., alloantigens) is administered a dosing regimen comprising a first dose administration of a population of modified cells described herein, a recovery period after the first dose, and a second dose administration of a population of modified cells described. In some embodiments, the composite of cell types present in the first population of cells and the second population of cells are different. In certain embodiments, the composite of cell types present in the first population of modified cells and the second population of modified cells are the same or substantially equivalent. In many embodiments, the first population of modified cells and the second population of modified cells comprises the same cell types. In some embodiments, the first population of modified cells and the second population of modified cells comprises different cell types. In some embodiments, the first population of modified cells and the second population of modified cells comprises the same percentages of cell types. In other embodiments, the first population of modified cells and the second population of cells comprises different percentages of cell types.

In some embodiments, the recovery period begins following the first administration of the population of modified cells or a composition containing the same, and ends when such cells are no longer present or detectable in the patient. In some embodiments, the duration of the recovery period is at least 1 week (e.g., 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, or more) or more after the initial administration of the cells. In some embodiments, the duration of the recovery period is at least 1 month (e.g., 1 month, 2 months, 3 months, 4 months, 5 months, 6 months, 7 months, 8 months, 9 months, 10 months, 11 months, 12 months, 13 months, 14 months, 15 months, 16 months, 17 months, 18 months, 19 months, 20 months, or more) or more after the initial administration of the cells.

In some embodiments, the administered population of modified cells, or a composition containing the same, is hypoimmunogenic when administered to the subject. In some embodiments, the modified cells are hypoimmune. In some embodiments, an immune response against the modified cells is reduced or lower by at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of the immune response produced by the administration of immunogenic cells (e.g. a population of cells of the same or similar cell type or phenotype but that do not contain the modifications, e.g. genetic modifications, of the modified cells). In some embodiments, the administered population of modified cells, or a composition containing the same, fails to elicit an immune response against the modified cells in the patient.

In some embodiments, the administered population of modified cells, or a composition containing the same, elicits a decreased or lower level of systemic TH1 activation in the patient. In some instances, the level of systemic TH1 activation elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of systemic TH1 activation produced by the administration of immunogenic cells (e.g. a population of cells of the same or similar cell type or phenotype but that do not contain the modifications, e.g. genetic modifications, of the modified cells). In some embodiments, the administered population of modified cells, or a composition containing the same, fails to elicit systemic TH1 activation in the patient.

In some embodiments, the administered population of modified cells, or a composition containing the same, elicits a decreased or lower level of immune activation of peripheral blood mononuclear cells (PBMCs) in the patient. In some instances, the level of immune activation of PBMCs elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of immune activation of PBMCs produced by the administration of immunogenic cells (e.g. a population of cells of the same or similar cell type or phenotype but that do not contain the modifications, e.g. genetic modifications, of the modified cells). In some embodiments, the administered population of modified cells, or a composition containing the same, fails to elicit immune activation of PBMCs in the patient.

In some embodiments, the administered population of modified cells, or a composition containing the same, elicits a decreased or lower level of donor-specific IgG antibodies in the patient. In some instances, the level of donor-specific IgG antibodies elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of donor-specific IgG antibodies produced by the administration of immunogenic cells (e.g. a population of cells of the same or similar cell type or phenotype but that do not contain the modifications, e.g. genetic modifications, of the modified cells). In some embodiments, the administered population of modified cells fails to elicit donor-specific IgG antibodies in the patient.

In some embodiments, the administered population of modified cells, or a composition containing the same, elicits a decreased or lower level of IgM and IgG antibody production in the patient. In some instances, the level of IgM and IgG antibody production elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of IgM and IgG antibody production produced by the administration of immunogenic cells (e.g. a population of cells of the same or similar cell type or phenotype but that do not contain the modifications, e.g. genetic modifications, of the modified cells). In some embodiments, the administered population of modified cells, or a composition containing the same, fails to elicit IgM and IgG antibody production in the patient.

In some embodiments, the administered population of modified cells, or a composition containing the same, elicits a decreased or lower level of cytotoxic T cell killing in the patient. In some instances, the level of cytotoxic T cell killing elicited by the cells is at least 5%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, or 99% lower compared to the level of cytotoxic T cell killing produced by the administration of immunogenic cells (e.g. a population of cells of the same or similar cell type or phenotype but that do not contain the modifications, e.g. genetic modifications, of the modified cells). In some embodiments, the administered population of modified cells, or a composition containing the same, fails to elicit cytotoxic T cell killing in the patient.

As discussed above, provided herein are cells that in certain embodiments can be administered to a patient sensitized against alloantigens such as human leukocyte antigens. In some embodiments, the patient is or has been pregnant, e.g., with alloimmunization in pregnancy (e.g., hemolytic disease of the fetus and newborn (HDFN), neonatal alloimmune neutropenia (NAN) or fetal and neonatal alloimmune thrombocytopenia (FNAIT)). In other words, the patient has or has had a disorder or condition associated with alloimmunization in pregnancy such as, but not limited to, hemolytic disease of the fetus and newborn (HDFN), neonatal alloimmune neutropenia (NAN), and fetal and neonatal alloimmune thrombocytopenia (FNAIT). In some embodiments, the patient has received an allogeneic transplant such as, but not limited to, an allogeneic cell transplant, an allogeneic blood transfusion, an allogeneic tissue transplant, or an allogeneic organ transplant. In some embodiments, the patient exhibits memory B cells against alloantigens. In some embodiments, the patient exhibits memory T cells against alloantigens. Such patients can exhibit both memory B and memory T cells against alloantigens.

Upon administration of the cells described, the patient exhibits no systemic immune response or a reduced level of systemic immune response compared to responses to cells that are not hypoimmunogenic. In some embodiments, the patient exhibits no adaptive immune response or a reduced level of adaptive immune response compared to responses to cells that are not hypoimmunogenic. In some embodiments, the patient exhibits no innate immune response or a reduced level of innate immune response compared to responses to cells that are not hypoimmunogenic. In some embodiments, the patient exhibits no T cell response or a reduced level of T cell response compared to responses to cells that are not hypoimmunogenic. In some embodiments, the patient exhibits no B cell response or a reduced level of B cell response compared to responses to cells that are not hypoimmunogenic.

The specific amount/dosage regimen will vary depending on the weight, gender, age and health of the individual; the formulation, the biochemical nature, bioactivity, bioavailability and the side effects of the cells and the number and identity of the cells in the complete therapeutic regimen.

The amount of cells used in implantation depends on a number of various factors including the patient's condition and response to the therapy, and can be determined by one skilled in the art. In some embodiments, the modified SB-beta cells are administered in an amount from or from about 1000 islet equivalent units (IEQ) to at or about 1×106 IEQ, such as from or from about 1000 IEG to at or about 500,000 IEQ, at or about 1000 IEQ to at or about 250,000 IEQ, at or about 1000 IEQ to at or about 100,000 IEQ, at or about 1000 IEQ to at or about 50,000 IEQ, at or about 1000 IEQ to at or about 25,000 IEQ, at or about 1000 IEQ to at or about 10000 IEQ, at or about 1000 IEQ to at or about 5000 IEQ, at or about 5000 IEQ to at or about 1×106 IEQ, at or about 5000 IEQ to at or about 500,000 IEQ, at or about 5000 IEQ to at or about 250,000 IEQ, at or about 5000 IEQ to at or about 100,000 IEQ, at or about 5000 IEQ to at or about 50,000 IEQ, at or about 5000 IEQ to at or about 250000 IEQ, at or about 5000 IEQ to at or about 10000 IEQ, at or about 10000 IEQ to at or about 1×106 IEQ, at or about 10000 IEQ to at or about 500000 IEQ, at or about 10000 IEQ to at or about 250000 IEQ, at or about 10000 IEQ to at or about 100000 IEQ, at or about 10000 IEQ to at or about 50000 IEQ, at or about 10000 IEQ to at or about 250000 IEQ, at or about 25000 IEQ to at or about 1×106 IEQ, at or about 25000 IEQ to at or about 500000 IEQ, at or about 25000 IEQ to at or about 250000 IEQ, at or about 25000 IEQ to at or about 100000 IEQ, at or about 25000 IEQ to at or about 50000 IEQ, at or about 50000 IEQ to at or about 1×106 IEQ, at or about 50000 IEQ to at or about 500000 IEQ, at or about 50000 IEQ to at or about 150000 IEQ, at or about 50000 IEQ to at or about 100000 IEQ, at or about 100000 IEQ to at or about 1×106 IEQ, at or about 100000 IEQ to at or about 500000 IEQ, at or about 100000 IEQ to at or about 250000 IEQ, at or about 250000 IEQ to at or about 1×106 IEQ, at or about 250000 IEQ to at or about 500000 IEQ, or at or about 500000 IEQ to at or about 1×106 IEQ. In some embodiments, the modified SB-beta cells are administered in an amount that is at or about 50,000 IEQ, at or about 100,000 IEQ, at or about 200,000 IEQ, at or about 300,000 IEQ, at or about 400,000 IEQ, or at or about 500,000 IEQ, or any value between any of the foregoing. IEQ provides a standardized estimate of islet volume, with one IEQ corresponding to the volume of a perfectly spherical islet with a diameter of 150 μm (Ricordi et al. Acta Diabetol. Lat. 27, 185-195 (1990).

In some embodiments, the total amount of cells is administered per kg of body weight. In some embodiments, islet cells are administered in a dosage amount of from at or about 500 IEQ/kg of body weight to at or about 10000 IEQ/kg, from at or about 500 IEQ/kg to at or about 5000 IEQ/kg, from at or about 500 IEQ/kg to at or about 2500 IEQ/kg, from at or about 500 IEQ/kg to at or about 1000 IEQ/kg, from at or about 1000 IEQ/kg to at or about 10000 IEQ/kg, from at or about 1000 IEQ/kg to at or about 5000 IEQ/kg, from at or about 1000 IEQ/kg to at or about 2500 IEQ/kg, from at or about 2500 IEQ/kg to at or about 10000 IEQ/kg, from at or about 2500 IEQ/kg to at or about 5000 IEQ/kg, or from at or about 5000 IEQ/kg to at or about 10000 IEQ/kg.

### A. Immunosuppressive Agent

In some embodiments, an immunosuppressive and/or immunomodulatory agent is not administered to the patient before the first administration of the population of modified cells, or in a composition containing the same.

In some embodiments, an immunosuppressive and/or immunomodulatory agent may be administered to a patient received administration of modified cells. In some embodiments, the immunosuppressive and/or immunomodulatory agent is administered prior to administration of the modified cells. In some embodiments, the immunosuppressive and/or immunomodulatory agent is administered prior to administration of a first and/or second administration of modified cells.

Non-limiting examples of an immunosuppressive and/or immunomodulatory agent include cyclosporine, azathioprine, mycophenolic acid, mycophenolate mofetil, corticosteroids such as prednisone, methotrexate, gold salts, sulfasalazine, antimalarials, brequinar, leflunomide, mizoribine, 15-deoxyspergualine, 6-mercaptopurine, cyclophosphamide, rapamycin, tacrolimus (FK-506), OKT3, anti-thymocyte globulin, thymopentin, thymosin-α and similar agents. In some embodiments, the immunosuppressive and/or immunomodulatory agent is selected from a group of immunosuppressive antibodies consisting of antibodies binding to p75 of the IL-2 receptor, antibodies binding to, for instance, MHC, CD2, CD3, CD4, CD7, CD28, B7, CD40, CD45, IFN-gamma, TNF-.alpha., IL-4, IL-5, IL-6R, IL-6, IGF, IGFR1, IL-7, IL-8, IL-10, CD11a, or CD58, and antibodies binding to any of their ligands. In some embodiments where an immunosuppressive and/or immunomodulatory agent is administered to the patient before or after the first administration of the cells, the administration is at a lower dosage than would be required for cells with MHC class I molecules and/or MHC class II molecules expression and without exogenous expression of CD47.

In one embodiment, such an immunosuppressive and/or immunomodulatory agent may be selected from soluble IL-15R, IL-10, B7 molecules (e.g., B7-1, B7-2, variants thereof, and fragments thereof), ICOS, and OX40, an inhibitor of a negative T cell regulator (such as an antibody against CTLA-4) and similar agents.

In some embodiments, an immunosuppressive and/or immunomodulatory agent can be administered to the patient before the first administration of the population of modified cells. In some embodiments, an immunosuppressive and/or immunomodulatory agent is administered at least 1, 2, 3, 4, 5, 6, 7, 8, 9, 10, 11, 12, 13, 14 days or more before the first administration of the cells. In some embodiments, an immunosuppressive and/or immunomodulatory agent is administered at least 1 week, 2 weeks, 3 weeks, 4 weeks, 5 weeks, 6 weeks, 7 weeks, 8 weeks, 9 weeks, 10 weeks or more before the first administration of the cells.

In particular embodiments, an immunosuppressive and/or immunomodulatory agent is not administered to the patient after the first administration of the cells, or is administered at least 1, 2, 3, 4, 5, 6, 7, 8, 9, 10, 11, 12, 13, 14 days or more after the first administration of the cells. In some embodiments, an immunosuppressive and/or immunomodulatory agent is administered at least 1 week, 2 weeks, 3 weeks, 4 weeks, 5 weeks, 6 weeks, 7 weeks, 8 weeks, 9 weeks, 10 weeks or more after the first administration of the cells.

In some embodiments, an immunosuppressive and/or immunomodulatory agent is not administered to the patient before the administration of the population of engineered cells. In many embodiments, an immunosuppressive and/or immunomodulatory agent is administered to the patient before the first and/or second administration of the population of modified cells. In some embodiments, an immunosuppressive and/or immunomodulatory agent is administered at least 1, 2, 3, 4, 5, 6, 7, 8, 9, 10, 11, 12, 13, 14 days or more before the administration of the cells. In some embodiments, an immunosuppressive and/or immunomodulatory agent is administered at least 1 week, 2 weeks, 3 weeks, 4 weeks, 5 weeks, 6 weeks, 7 weeks, 8 weeks, 9 weeks, 10 weeks or more before the first and/or second administration of the cells. In particular embodiments, an immunosuppressive and/or immunomodulatory agent is administered at least 1, 2, 3, 4, 5, 6, 7, 8, 9, 10, 11, 12, 13, 14 days or more after the administration of the cells. In some embodiments, an immunosuppressive and/or immunomodulatory agent is administered at least 1 week, 2 weeks, 3 weeks, 4 weeks, 5 weeks, 6 weeks, 7 weeks, 8 weeks, 9 weeks, 10 weeks or more after the first and/or second administration of the cells.

In some embodiments where an immunosuppressive and/or immunomodulatory agent is administered to the patient before or after the administration of the cells, the administration is at a lower dosage than would be required for immunogenic cells (e.g. a population of cells of the same or similar cell type or phenotype but that do not contain the modifications, e.g. genetic modifications, of the modified cells, e.g. with endogenous levels of CD142, MHC class I molecules, and/or MHC class II molecules expression and without increased (e.g., exogenous) expression of CD47).

### B. Anti-Coagulant Agent

In some embodiments, an anti-coagulant agent is administered to the patient during and/or after administration of the population of modified cells. In some embodiments, an anti-coagulant agent and the population or modified cells are administered to the patient simultaneously. In some embodiments, the anti-coagulant agent and the population of modified cells are administered sequentially.

In some embodiments, a method provided herein comprises administering to a subject (i) a population of modified cells described herein, and (ii) an anti-coagulant agent. In some embodiments, the anti-coagulant agent is any of the anti-coagulant agents described below.

In some embodiments the anti-coagulant agent is administered at the time of administering the modified cell population, wherein the heparin is administered at a dose of 70 U/kg recipient body weight. In some embodiments, the anti-coagulant agent is provided in a separate formulation and is administered simultaneously with the population of modified cells. In some embodiments, the anti-coagulant agent and the population of modified cells are provided in separate infusion bags, e.g., for intravenous administration. In some embodiments, the anti-coagulant agent is heparin. In some embodiments, the heparin is administered systemically. In some embodiments, the heparin is administered by infusion to the portal vein via the mesenteric vein. In some embodiments, after 2, 3, 4, 5, 6, 7, 8, 9, or 10 hours post surgery, patients are continuously infused with heparin for 12-36 hours (e.g., for about 24 hours). In some embodiments, the dosage of heparin administered during the post-surgery period is lower than the initial administration of heparin. In some embodiments, heparin is administered post surgery at a dose of 200-400 U/h (e.g., a dose of about 300 U/h) heparin. In some hours, heparin is administered by continuous infusion at a dose of 300 U/h heparin for 24 h after surgery. In some embodiments, heparin is administered by continuous infusion at a dose of 300 U/h heparin for 24 h after surgery beginning 6 hours after surgery.

In some embodiments, heparin is administered at the time of administering the modified cell population, wherein the heparin is administered at a dose of 70 U/kg recipient body weight. In some embodiments, the heparin is administered systemically. In some embodiments, the heparin is administered by infusion to the portal vein via the mesenteric vein. In some embodiments, after 2, 3, 4, 5, 6, 7, 8, 9, or 10 hours post surgery, patients are continuously infused with heparin for 12-36 hours (e.g., for about 24 hours). In some embodiments, the dosage of heparin administered during the post-surgery period is lower than the initial administration of heparin. In some embodiments, heparin is administered post surgery at a dose of 200-400 U/h (e.g., a dose of about 300 U/h) heparin. In some hours, heparin is administered by continuous infusion at a dose of 300 U/h heparin for 24 h after surgery. In some embodiments, heparin is administered by continuous infusion at a dose of 300 U/h heparin for 24 h after surgery beginning 6 hours after surgery.

In some embodiments, the anti-coagulant is selected from the group consisting heparin, an activator of antithrombin, an inhibitor of coagulation factor II (fII), an inhibitor of coagulation factor VII (fVII), and an inhibitor of coagulation factor X (fX). In some embodiments, the anti-coagulant agent is heparin. In some embodiments, the heparin is unfractionated heparin. In some embodiments, the heparin is low molecular weight heparin. In some embodiments, the heparin is soluble heparin. In some embodiments, the anti-coagulant is melagatran or LMW-DS. In some embodiments, wherein the anti-coagulant is N-acetylcysteine (NAC). In some embodiments, the anti-coagulant is alpha-1 antitrypsin (AAT) and/or activated protein C. In some embodiments, the anti-coagulant is an antibody against CD142.

In some embodiments, the anti-coagulant is administered systemically. In some embodiments, the anti-coagulant is administered by IV infusion.

### V. Exemplary Embodiments

Among the provided embodiments are:

1. A method of generating a modified stem cell derived beta cell (SC-beta cell), the method comprising:
(A) providing a modified pluripotent stem cell (PSC) comprising modifications that:
   (a) reduce expression of one or more of major histocompatibility complex (MHC) class I molecules and/or one or more of MHC class II molecules in the modified PSC, relative to a control or wild-type PSC; and
   (b) increase expression of one or more tolerogenic factors in the modified PSC, relative to the control or wild-type PSC; and
(B) culturing the modified PSC under conditions sufficient for differentiation of the modified PSC into the modified SC-beta cell.

2. A method of generating a modified stem cell derived beta cell (SC-beta cell) the method comprising:
(A) generating a modified pluripotent stem cell (PSC) comprising:
   (a) reducing expression of one or more of major histocompatibility complex (MHC) class I molecules and/or one or more MHC class II molecules in a PSC, relative to a control or wild-type PSC; and
   (b) increasing expression of one or more tolerogenic factors in the PSC, relative to the control or wild-type PSC; and
(B) culturing the modified PSC under conditions sufficient for differentiation of the modified PSC into a modified SC-beta cell.

3. The method of embodiment 2, wherein in (a) reducing expression of the one or more MHC class I molecules and/or the one or more MHC class II molecules comprises introducing modifications that reduce expression of one or more MHC class I molecules and/or one or more MHC class II molecules in the modified PSC, relative to the control or wild-type PSC.

4. The method of any of embodiments 1-3, wherein the control or wild-type PSC is an unmodified PSC that does not comprise the modifications.

5. The method of any of embodiments 1-3, wherein expression of one or more MHC class I molecules and one or more MHC class II molecules is reduced in the modified PSC.

6. The method of any of embodiments 1 and 3-5, wherein the modifications in (a) reduce protein expression of the one or more MHC class I molecules and/or wherein the modifications in (a) reduce cell surface expression of the one or more MHC class I molecules.

7 The method of any of embodiments 1 and 3-6, wherein the modifications in (a) reduce a function of the one or more MHC class I molecules, optionally wherein the function is antigen presentation.

8. The method of any of embodiments 1-7, wherein the one or more MHC class I molecules is one or more human leukocyte antigen (HLA) class I molecules.

9. The method of any of embodiments 1-8, wherein the one or more MHC HLA class I molecules is selected from the group consisting of HLA-A, HLA-B, and HLA-C

10. The method of any of embodiments 1-9, wherein the modification that reduces expression of the one or more MHC class I molecules reduces expression of B2M.

11. The method of any of embodiments 1-10, wherein the modification that reduces expression of the one or more MHC class I molecules reduces mRNA expression of the B2M gene.

12. The method of any of embodiments 1-10, wherein the modification that reduces expression of the one or more MHC class I molecules reduces protein expression of B2M.

13. The method of any of embodiments 1-12, wherein the modification that reduces expression of the one or more MHC class I molecules comprises:
inactivation or disruption of one allele of the B2M gene;
inactivation or disruption of both alleles of the B2M gene; or
inactivation or disruption of all B2M coding alleles in the cell.

14. The method of embodiment 13, wherein the inactivation or disruption comprises an indel in the B2M gene.

15. The method of embodiment 13 or embodiment 14, wherein the inactivation or disruption comprises a frameshift mutation or a deletion of a contiguous stretch of genomic DNA of the B2M gene.

16. The method of any of embodiments 1-15, wherein the modifications in (a) reduce protein expression of the one or more MHC class II molecules.

17. The method of any of embodiments 1-16, wherein the modifications in (a) reduce cell surface expression of the one or more MHC class II molecules.

18. The method of any of embodiments 1-17, wherein the modifications in (a) reduce a function of the one or more MHC class II molecules, optionally wherein the function is antigen presentation.

19. The method of any of embodiments 1-18, wherein the one or more MHC class II molecules is one or more human leukocyte antigen (HLA) class II molecules.

20. The method of any of embodiments 1-19, wherein the one or more MHC HLA class II molecules is selected from the group consisting of HLA-DP, HLA-DQ, and/or HLA-DR.

21. The method of any of embodiments 1-20, wherein the modification that reduces expression of the one or more MHC class II molecules comprises reduced expression of CIITA.

22. The method of any of embodiments 1-21, wherein the modification that reduces expression of the one or more MHC class II molecules reduces mRNA expression of the CIITA gene.

23. The method of any of embodiments 1-21, wherein the modification that reduces expression of the one or more MHC class II molecules reduces protein expression of CIITA.

24. The method of any of embodiments 1-23, wherein the modification that reduces expression of the one or more MHC class II molecules comprises:
inactivation or disruption of one allele of the CIITA gene;
inactivation or disruption of both alleles of the CIITA gene; or
inactivation or disruption of all CIITA coding alleles in the cell.

25. The method of embodiment 24, wherein the inactivation or disruption comprises an indel in the CIITA gene.

26. The method of embodiment 24 or embodiment 25, wherein the inactivation or disruption is a frameshift mutation or a deletion of a contiguous stretch of genomic DNA of the CIITA gene.

27. The method of any of embodiments 1-26, wherein the one or more tolerogenic factors is selected from the group consisting of CD47, CD27, CD200, HLA-C, HLA-E, HLA-E heavy chain, HLA-G, PD-L1, IDO1, CTLA4-Ig, Cl-Inhibitor, IL-10, IL-35, FASL, CCL21, MFGE8, and SERPINB9.

28. The method of any of embodiments 1-27, wherein at least one of the one or more tolerogenic factors is CD47.

29. The method of any of embodiments 1-28, wherein the one or more tolerogenic factors is CD47.

30. The method of any of embodiments 1-28, wherein at least one of the one or more tolerogenic factors is PD-L1.

31. The method of any of embodiments 1-30, wherein at least one of the one or more tolerogenic factors is HLA-E.

32. The method of any of embodiments 1-31, wherein at least one of the one or more tolerogenic factors is HLA-G.

33. The method of any of 2-32, wherein increasing expression of the one or more tolerogenic factors comprises introducing a modification that increases expression of the one or more tolerogenic factor in the modified PSC, relative to the control or wild-type PSC.

34. The method of any of 1 and 3-33, wherein the modification to increase expression of the one or more tolerogenic factors comprises an exogenous polynucleotide encoding the one or more tolerogenic factors.

35. The method of embodiment 34, wherein the exogenous polynucleotide encoding the one or more tolerogenic factors is integrated into the genome of the modified PSC.

36. The method of embodiment 35, wherein the exogenous polynucleotide encoding the one or more tolerogenic factors is integrated by non-targeted insertion into the genome of the modified PSC, optionally by introduction of the exogenous polynucleotide into the cell using a lentiviral vector.

37. The method of embodiment 35, wherein the exogenous polynucleotide encoding the one or more tolerogenic factors is integrated by targeted insertion into a target genomic locus of the cell, optionally wherein the targeted insertion is by nuclease-mediated gene editing with homology-directed repair.

38. A method of generating a modified stem cell derived beta cell (SC-beta cell), the method comprising:
(A) providing a modified pluripotent stem cell (PSC) comprising knock out of the B2M gene, knock out of the CIITA gene, and an exogenous polynucleotide encoding CD47 protein, relative to a control or wild-type PSC; and
(B) culturing the modified PSC under conditions sufficient for differentiation of the modified PSC into the modified SC-beta cell.

39. The method of embodiment 38, wherein the modified PSC has the phenotype B2M^{indel/indel}, CIITA^{indel/indel}; CD47tg.

40. A method of generating a modified stem cell derived beta cell (SC-beta cell), the method comprising:
(A) providing a modified pluripotent stem cell (PSC) comprising knock out of the B2M gene, knock out of the CIITA gene, an exogenous polynucleotide encoding CD47 protein, and an exogenous polynucleotide encoding a suicide gene, relative to a control or wild-type PSC; and
(B) culturing the modified PSC under conditions sufficient for differentiation of the modified PSC into the modified SC-beta cell.

41. The method of embodiment 40, wherein the modified PSC has the phenotype B2M^{indel/indel}, CIITA^{indel/indel}; CD47tg; suicide genetg.

42. The method of any of embodiments 38-41, wherein the exogenous polynucleotide encoding CD47 is integrated by non-targeted insertion into the genome of the modified PSC, optionally by introduction of the exogenous polynucleotide into the cell using a lentiviral vector.

43. The method of any of embodiments 38-41, wherein the exogenous polynucleotide encoding CD47 is integrated by targeted insertion into a target genomic locus of the cell, optionally wherein the targeted insertion is by nuclease-mediated gene editing with homology-directed repair.

44. The method of any of embodiment 1-39, wherein the modified PSC further comprises a modification to increase expression of an exogenous suicide gene.

45. The method of any of embodiments 40-44, wherein the suicide gene is selected from the group consisting of cytosine deaminase (CyD), herpesvirus thymidine kinase (HSV-Tk), an inducible caspase 9 (iCaspase9), and rapamycin-activated caspase 9 (rapaCasp9).

46. The method of embodiment 44 or embodiment 45, wherein the suicide gene and the one or more tolerogenic factors are expressed from a bicistronic cassette integrated into the genome of the modified PSC.

47. The method of any of embodiments 40-43, wherein the suicide gene and CD47 are expressed from a bicistronic cassette integrated into the genome of the modified PSC.

48. The method of embodiment 46 and 47, wherein the bicistronic cassette is integrated by non-targeted insertion into the genome of the modified PSC, optionally by introduction of the exogenous polynucleotide into the cell using a lentiviral vector.

49. The method of embodiment 46 or embodiment 47, wherein the bicistronic cassette is integrated by targeted insertion into a target genomic locus of the cell, optionally wherein the targeted insertion is by nuclease-mediated gene editing with homology-directed repair.

50. The method of embodiment 43 and embodiment 49, wherein the target genomic locus is a safe harbor locus, a B2M gene locus, a CIITA gene locus, or a CD142 gene locus.

51. The method of embodiment 50, wherein the safe harbor locus is selected from the group consisting of: a CCR5 gene locus, a CXCR4 gene locus, a PPP1R12C (also known as AAVS1) gene, an albumin gene locus, a SHS231 locus, a CLYBL gene locus, and a ROSA26 gene locus.

52. The method of any of embodiments 1-51, wherein the modified PSC comprises a modification that reduces expression of CD142, relative to the control or wild-type PSC.

53. The method of embodiment 52, wherein the modification reduces mRNA expression of the CD142 gene.

54. The method of embodiment 52, wherein the modification reduces protein expression of CD142.

55. The method of any of embodiments 52-54, wherein the modification comprises:
inactivation or disruption of one allele of the CD142 gene;
inactivation or disruption of both alleles of the CD142 gene;
inactivation or disruption of all CD142 coding alleles in the cell.

56. The method of embodiment 55, wherein the inactivation or disruption comprises an indel in the CD142 gene.

57. The method of embodiment 55 or embodiment 56, wherein the inactivation or disruption is a frameshift mutation or a deletion of a contiguous stretch of genomic DNA of the CD142 gene.

58. The method of any of embodiments 1-57, wherein the modified PSC comprises a modification that increases expression of one or more complement inhibitors selected from the group consisting of CD46, CD59, CD55, and CD35, relative to the control or wild-type PSC.

59. The method of embodiment 58, wherein the modification to increase expression of the one or more complement inhibitors comprises at least one exogenous polynucleotide selected from the group consisting of an exogenous polynucleotide encoding CD46, an exogenous polynucleotide encoding CD59, an exogenous polynucleotide encoding CD55, and an exogenous polynucleotide encoding CD35.

60. The method of embodiment 58 or embodiment 59, wherein the one or more complement inhibitors is CD46 and CD59.

61. The method of embodiment 58 or embodiment 59, wherein the one or more complement inhibitor is CD46, CD59 and CD55.

62. The method of any of embodiments 59-61, wherein the at least one exogenous polynucleotide is integrated by non-targeted insertion into the genome of the modified PSC, optionally by introduction of the exogenous polynucleotide into the cell using a lentiviral vector.

63. The method of any of embodiments 59-61, wherein the at least one exogenous polynucleotide is integrated by targeted insertion into a target genomic locus of the cell, optionally wherein the targeted insertion is by nuclease-mediated gene editing with homology-directed repair.

64. The method of embodiment 63, wherein the target genomic locus is a safe harbor locus, a B2M gene locus, a CIITA gene locus, or a CD142 gene locus.

65. The method of embodiment 64, wherein the safe harbor locus is selected from the group consisting of: a CCR5 gene locus, a CXCR4 gene locus, a PPP1R12C (also known as AAVS1) gene, an albumin gene locus, a SHS231 locus, a CLYBL gene locus, and a ROSA26 gene locus.

66. The method of any of embodiments 1-65, wherein the culturing the modified PSC under conditions sufficient for differentiation of the modified PSC into the modified SC-beta cell comprises one or more of:
(i) contacting the modified PSC with a TGF/Activin agonist or a glycogen synthase kinase 3 (GSK) inhibitor or WNT agonist for an amount of time sufficient to form a definitive endoderm cell;
(ii) contacting a definitive endoderm cell differentiated from the modified PSC with a FGFR2b agonist for an amount of time sufficient to form a primitive gut tube cell;
(iii) contacting a primitive gut tube cell differentiated from the modified PSC with an RAR agonist, and optionally a rho kinase inhibitor, a smoothened antagonist, a FGFR2b agonist, a protein kinase C activator, or a BMP type 1 receptor inhibitor for an amount of time sufficient to form an early pancreas progenitor cell;
(iv) incubating an early pancreas progenitor cell differentiated from the modified PSC for at least about 3 days and optionally contacting the early pancreas progenitor cell with a rho kinase inhibitor, a TGF-/Activin agonist, a smoothened antagonist, an FGFR2b agonist, or a RAR agonist for an amount of time sufficient to form a pancreatic progenitor cell;
(v) contacting a pancreatic progenitor cell differentiated from the modified PSC with an Alk5 inhibitor, a gamma secretase inhibitor, SANT 1, Erbb1 (EGFR) or Erbb4 agonist, or a RAR agonist for an amount of time sufficient to form an endoderm cell, wherein during at least a portion of the contacting in (v) depolymerizing the actin cytoskeleton at a time and for an amount of time sufficient to increase differentiation efficiency; and/or
(vi) incubating an endoderm cell differentiated from the modified PSC for an amount of time in serum-free media sufficient to form a beta cell, and within about 24 hours of incubation resizing the beta cells that formed into beta cell clusters.

67. The method of any of embodiments 1-65, wherein the culturing the modified PSC under conditions sufficient for differentiation of the modified PSC into the modified SC-beta cell comprises:
(i) contacting the modified PSC with a TGF/Activin agonist or a glycogen synthase kinase 3 (GSK) inhibitor or WNT agonist for an amount of time sufficient to form a definitive endoderm cell;
(ii) contacting the definitive endoderm cell with a FGFR2b agonist for an amount of time sufficient to form a primitive gut tube cell;
(iii) contacting the primitive gut tube cell with an RAR agonist, and optionally a rho kinase inhibitor, a smoothened antagonist, a FGFR2b agonist, a protein kinase C activator, or a BMP type 1 receptor inhibitor for an amount of time sufficient to form an early pancreas progenitor cell;
(iv) incubating the early pancreas progenitor cell for at least about 3 days and optionally contacting the early pancreas progenitor cell with a rho kinase inhibitor, a TGF-/Activin agonist, a smoothened antagonist, an FGFR2b agonist, or a RAR agonist for an amount of time sufficient to form a pancreatic progenitor cell;
(v) contacting the pancreatic progenitor cell with an Alk5 inhibitor, a gamma secretase inhibitor, SANT 1, Erbb1 (EGFR) or Erbb4 agonist, or a RAR agonist for an amount of time sufficient to form an endoderm cell, wherein during at least a portion of the contacting in (v) depolymerizing the actin cytoskeleton at a time and for an amount of time sufficient to increase differentiation efficiency; and
(vi) incubating the endoderm cell for an amount of time in serum-free media sufficient to form a beta cell, and within about 24 hours of incubation resizing the beta cells that formed into beta cell clusters.

68. The method of embodiment 66 or embodiment 67, wherein depolymerizing the actin cytoskeleton comprises plating cells on a stiff or soft substrate or introducing a cytoskeletal-modulating agent to cells.

69. The method of embodiment 68, wherein the cytoskeletal-modulating agent comprises latrunculin A, latrunculin B, nocodazole, cytochalasin D, jasplakinolide, blebbistatin, y-27632, y-15, gdc-0994, or an integrin modulating agent.

70. The method of embodiment 68 or embodiment 69, wherein the cytoskeletal-modulating agent is latrunculin A.

71. The method of any of embodiments 66-70, wherein depolymerizing the actin cytoskeleton is initiated at the start of the contacting in (v).

72. The method of any of embodiments 66-71, wherein depolymerizing the actin cytoskeleton comprises adding latrunculin A at the start of the contacting for at least at or about the first 24 hours.

73. The method of any of embodiments 66-72, wherein resizing the beta cell clusters comprises breaking apart clusters and reaggregating.

74. The method of any of embodiments 66-73, wherein:
the TGF/Activin agonist is Activin A;
the glycogen synthase kinase 3 (GSK) inhibitor or the WNT agonist is CHIR;
the FGFR2b agonist is KGF;
the smoothened antagonist is SANT-1;
the RAR agonist is retinoic acid (RA);
the protein kinase C activator is TPPB;
the BMP type 1 receptor inhibitor is LDN;
the rho kinase inhibitor is Y27632;
the Alk5 inhibitor is Alk51;
the Erbb4 agonist is betacellulin;
the thyroid hormone is T3; and/or
the gamma secretase inhibitor is XXI.

75. The method of any of embodiments 1-74, wherein the PSC is an embryonic stem cell.

76. The method of any of embodiments 1-74, wherein the PSC is an induced PSC (iPSC), optionally a patient-derived iPSC.

77. The method of any of embodiments 1-76, wherein the modified PSC expresses each of the one or more tolerogenic factors at a first level that is greater than at or about 5-fold over a second level expressed by the control or wild-type PSC.

78. The method of embodiment 77, wherein each of the one or more tolerogenic factors is expressed at a first level that is greater than at or about 10-fold, greater than at or about 20-fold, greater than at or about 30-fold, greater than at or about 40-fold, greater than at or about 50-fold, greater than at or about 60-fold, or greater than at or about 70-fold over a second level expressed by the control or wild-type PSC.

79. The method of any of embodiments 1-78, wherein each of the one or more tolerogenic factors is expressed by the modified PSC at greater than at or about 20,000 molecules per cell.

80. The method of embodiment 79, wherein each of the one or more tolerogenic factors is expressed by the modified PSC at greater than at or about 30,000 molecules per cell, greater than at or about 50,000 molecules per cell, greater than at or about 100,000 molecules per cell, greater than at or about 200,000 molecules per cell, greater than at or about 300,000 molecules per cell, greater than at or about 400,000 molecules per cell, greater than at or about 500,000 molecules per cell, or greater than at or about 600,000 molecules per cell.

81. The method of any of embodiments 1-76, wherein the one or more tolerogenic factors comprises CD47 and the modified PSC expresses CD47 at a first level that is greater than at or about 5-fold over a second level expressed by the control or wild-type PSC.

82. The method of embodiment 81, wherein CD47 is expressed at a first level that is greater than at or about 10-fold, greater than at or about 20-fold, greater than at or about 30-fold, greater than at or about 40-fold, greater than at or about 50-fold, greater than at or about 60-fold, or greater than at or about 70-fold over a second level expressed by the control or wild-type PSC.

83. The method of any of embodiments 1-80, 81 and 82, wherein the one or more tolerogenic factors comprises CD47 and CD47 is expressed by the modified PSC at greater than at or about 20,000 molecules per cell.

84. The method of embodiment 83, wherein CD47 is expressed by the modified PSC at greater than at or about 30,000 molecules per cell, greater than at or about 50,000 molecules per cell, greater than at or about 100,000 molecules per cell, greater than at or about 200,000 molecules per cell, greater than at or about 300,000 molecules per cell, greater than at or about 400,000 molecules per cell, greater than at or about 500,000 molecules per cell, or greater than at or about 600,000 molecules per cell.

85. The method of any of embodiments 1-84, wherein the modified SC-beta cell comprises modifications that (1) reduce expression of one or more MHC class I molecules and/or one or more MHC class II molecules, relative to a control or wild-type beta cell; and (2) increase expression of one or more tolerogenic factors, relative to the control or wild-type beta cell.

86. The method of embodiment 85, wherein the control or wild-type beta cell is an unmodified SC-beta cell differentiated from an unmodified PSC not comprising modifications that reduce expression of the one or more MHC class I molecules and/or the one or more MHC class II molecules or that increase expression of the one or more tolerogenic factors, relative to the control or wild-type PSC.

87. The method of embodiment 85 or embodiment 86, wherein expression of one or more MHC class I molecules and one or more MHC class II molecules is reduced in the modified SC-beta cell.

88. The method of any of embodiments 85-87, wherein the modifications in (1) reduce protein expression of the one or more MHC class I molecules.

89. The method of any of embodiment 85-88, wherein the modifications in (1) reduce cell surface expression of the one or more MHC class I molecules.

90. The method of any of embodiments 85-89, wherein the modifications in (1) reduce a function of the one or more MHC class I molecules, optionally wherein the function is antigen presentation.

91. The method of any of embodiments 85-90, wherein the modification that reduces expression of the one or more MHC class I molecules in the modified SC-beta cell comprises reduced expression of B2M.

92. The method of any of embodiments 85-91, wherein the modification that reduces expression of the one or more MHC class I molecules in the modified SC-beta cell reduces mRNA expression of the B2M gene.

93. The method of any of embodiments 85-92, wherein the modification that reduces expression of the one or more MHC class I molecules in the modified SC-beta cell reduces protein expression of B2M.

94. The method of any of embodiments 85-93, wherein the modification that reduces expression of the one or more MHC class I molecules in the modified SC-beta cell comprises:
inactivation or disruption of one allele of the B2M gene;
inactivation or disruption of both alleles of the B2M gene; or
inactivation or disruption of all B2M coding alleles in the cell.

95. The method of embodiment 94, wherein the inactivation or disruption comprises an indel in the B2M gene.

96. The method of embodiment 94 or embodiment 95, wherein the inactivation or disruption comprises a frameshift mutation or a deletion of a contiguous stretch of genomic DNA of the B2M gene.

97. The method of any of embodiments 85-96, wherein the modifications in (1) reduce protein expression of the one or more MHC class II molecules.

98. The method of any of embodiments 85-97, wherein the modifications in (1) reduce cell surface expression of the one or more MHC class II molecules.

99. The method of any of embodiments 85-98, wherein the modifications in (1) reduce a function of the one or more MHC class II molecules, optionally wherein the function is antigen presentation.

100. The method of any of embodiments 85-99, wherein the modification that reduces expression of the one or more MHC class II molecules in the modified SC-beta cell comprises reduced expression of CIITA.

101. The method of any of embodiments 85-100, wherein the modification that reduces expression of the one or more MHC class II molecules in the modified SC-beta cell reduces mRNA expression of the CIITA gene.

102. The method of any of embodiments 85-101, wherein the modification that reduces expression of the one or more MHC class II molecules in the modified SC-beta cell reduces protein expression of CIITA.

103. The method of any of embodiments 85-102, wherein the modification that reduces expression of the one or more MHC class II molecules comprises:
inactivation or disruption of one allele of the CIITA gene;
inactivation or disruption of both alleles of the CIITA gene; or
inactivation or disruption of all CIITA coding alleles in the cell.

104. The method of embodiment 103, wherein the inactivation or disruption comprises an indel in the CIITA gene.

105. The method of embodiment 103 or embodiment 104, wherein the inactivation or disruption is a frameshift mutation or a deletion of a contiguous stretch of genomic DNA of the CIITA gene.

106. The method of any of embodiments 1-105, wherein the modified SC-beta cell comprises a modification that reduces expression of CD142, relative to the control or wild-type beta cell.

107. The method of embodiment 106, wherein the modification reduces mRNA expression of the CD142 gene.

108. The method of embodiment 106, wherein the modification reduces protein expression of CD142.

109. The method of any of embodiments 106-108, wherein the modification comprises:
inactivation or disruption of one allele of the CD142 gene;
inactivation or disruption of both alleles of the CD142 gene; or
inactivation or disruption of all CD142 coding alleles in the cell.

110. The method of any of embodiments 106-109, wherein the inactivation or disruption comprises an indel in the CD142 gene.

111. The method of any of embodiments 106-110, wherein the inactivation or disruption is a frameshift mutation or a deletion of a contiguous stretch of genomic DNA of the CD142 gene.

112. The method of any of embodiments 1-111, wherein the modification to increase expression of the one or more tolerogenic factors in the modified SC-beta cell comprises an exogenous polynucleotide encoding the one or more tolerogenic factors.

113. The method of embodiment 112, wherein the exogenous polynucleotide encoding the one or more tolerogenic factors is integrated into the genome of the modified SC-beta cell.

114. The method of embodiment 113, wherein the exogenous polynucleotide is integrated into a non-target locus in the genome of the modified SC-beta cell.

115. The method of embodiment 113, wherein the exogenous polynucleotide is integrated into a target genomic locus of the modified SC-beta cell.

116. The method of any of embodiment 1-115, wherein the modified SC-beta cell further comprises a modification for expression of an exogenous suicide gene selected from the group consisting of cytosine deaminase (CyD), herpesvirus thymidine kinase (HSV-Tk), an inducible caspase (iCaspase9), and rapamycin-activated caspase 9 (rapaCasp9).

117. The method of embodiment 116, wherein the suicide gene and the one or more tolerogenic factors are expressed from a bicistronic cassette integrated into the genome of the modified SC-beta cell.

118. The method of embodiment 117, wherein the bicistronic cassette is integrated at a non-target locus in the genome of the modified SC-beta cell.

119. The method of embodiment 117, wherein the bicistronic cassette is integrated into a target genomic locus of the cell.

120. The method of embodiment 115 or embodiment 119, wherein the target genomic locus is a safe harbor locus, a B2M gene locus, a CIITA gene locus, or a CD142 gene locus.

121. The method of embodiment 120, wherein the safe harbor locus is selected from the group consisting of: a CCR5 gene locus, a CXCR4 gene locus, a PPP1R12C (also known as AAVS1) gene, an albumin gene locus, a SHS231 locus, a CLYBL gene locus, and a ROSA26 gene locus.

122. The method of any of embodiments 1-121, wherein the modified SC-beta cell comprises a modification that increases expression of one or more complement inhibitors selected from the group consisting of CD46, CD59, CD55, and CD35 relative to the control or wild-type beta cell.

123. The method of embodiment 122, wherein the modification to increase expression of the one or more complement inhibitors in the modified SC-beta cell comprises at least one exogenous polynucleotide encoding the one or more complement inhibitors selected from the group consisting of an exogenous polynucleotide encoding CD46, an exogenous polynucleotide encoding CD59, an exogenous polynucleotide encoding CD55, and an exogenous polynucleotide encoding CD35.

124. The method of embodiment 122 or embodiment 123, wherein the one or more complement inhibitors is CD46 and CD59.

125. The method of embodiment 122 or embodiment 123, wherein the one or more complement inhibitor is CD46, CD59 and CD55.

126. The method of any of embodiments 1-125, wherein the reduced expression comprises reduced surface expression.

127. The method of any of embodiments 1-126, wherein the increased expression comprises increased surface expression.

128. The method of any of embodiments 85-127, wherein the level of the reduced expression of (1) and the increased expression of (2) by the modified SC-beta cell is retained or is similar compared to the modified PSC.

129. The method of any of embodiments 85-128, wherein the modified SC-beta cell expresses the one or more tolerogenic factors at a first level that is greater than at or about 5-fold over a second level expressed by the control or wild-type beta cell, optionally wherein the control or wild-type beta cell is differentiated from an unmodified PSC not comprising modifications that reduce expression of the one or more MHC class I molecules and/or the one or more MHC class II molecules and that increase expression of the one or more tolerogenic factors.

130. The method of any of embodiments 85-129, wherein the modified SC-beta cell expresses each of the one or more tolerogenic factors at a first level that is greater than at or about 5-fold over a second level expressed by the control or wild-type beta cell.

131. The method of embodiment 130, wherein each of the one or more tolerogenic factor is expressed at a first level that is greater than at or about 10-fold, greater than at or about 20-fold, greater than at or about 30-fold, greater than at or about 40-fold, greater than at or about 50-fold, greater than at or about 60-fold, or greater than at or about 70-fold over a second level expressed by the control or wild-type beta cell.

132. The method of any of embodiments 85-131, wherein each of the one or more tolerogenic factors is expressed by the modified SC-beta cell at greater than at or about 20,000 molecules per cell.

133. The method of embodiment 132, wherein each of the one or more tolerogenic factors is expressed by the modified SC-beta cell at greater than at or about 30,000 molecules per cell, greater than at or about 50,000 molecules per cell, greater than at or about 100,000 molecules per cell, greater than at or about 200,000 molecules per cell, greater than at or about 300,000 molecules per cell, greater than at or about 400,000 molecules per cell, greater than at or about 500,000 molecules per cell, or greater than at or about 600,000 molecules per cell.

134. The method of any of embodiments 85-128, wherein the one or more tolerogenic factors comprises CD47 and the modified SC-beta cell expresses CD47 at a first level that is greater than at or about 5-fold over a second level expressed by the control or wild-type beta cell, optionally wherein the control or wild-type beta cell is differentiated from an unmodified PSC not comprising modifications that reduce expression of the one or more MHC class I molecules and/or the one or more MHC class II molecules and that increase expression of the one or more tolerogenic factors.

135. The method of any of embodiments 85-128 and 134, wherein the one or more tolerogenic factors comprises CD47 and the modified SC-beta cell expresses CD47 at a first level that is greater than at or about 5-fold over a second level expressed by the control or wild-type beta cell.

136. The method of embodiment 135, wherein CD47 is expressed at a first level that is greater than at or about 10-fold, greater than at or about 20-fold, greater than at or about 30-fold, greater than at or about 40-fold, greater than at or about 50-fold, greater than at or about 60-fold, or greater than at or about 70-fold over a second level expressed by the control or wild-type beta cell.

137. The method of any of embodiments 85-128 and 134-136, wherein the one or more tolerogenic factors comprises CD47 and CD47 is expressed by the modified SC-beta cell at greater than at or about 20,000 molecules per cell.

138. The method of embodiment 137, wherein CD47 is expressed by the modified SC-beta cell at greater than at or about 30,000 molecules per cell, greater than at or about 50,000 molecules per cell, greater than at or about 100,000 molecules per cell, greater than at or about 200,000 molecules per cell, greater than at or about 300,000 molecules per cell, greater than at or about 400,000 molecules per cell, greater than at or about 500,000 molecules per cell, or greater than at or about 600,000 molecules per cell.

139. The method of any of embodiments 1-138, wherein the modified SC-beta cell expresses at least one beta cell marker, optionally wherein the at least one beta cell marker is selected from the group consisting of INS, CHGA, NKX2-2, PDX1, NKX6-1, MAFB, GCK and GLUT1.

140. The method of any of embodiments 1-139, wherein the modified SC-beta cell exhibits one or more functions of a wild-type or control beta cell, optionally wherein the one or more functions is selected from the group consisting of in vitro glucose-stimulated insulin secretion (GSIS), glucose metabolism, maintaining fasting blood glucose levels, secreting insulin in response to glucose injections in vivo, and clearing glucose after a glucose injection in vivo.

141. The method of any of embodiments 1-140, wherein the modified SC-beta cell is capable of glucose-stimulated insulin secretion (GSIS), optionally wherein the insulin secretion is in a perfusion GSIS assay.

142. The method of embodiment 141, wherein the GSIS is dynamic GSIS comprising first and second phase dynamic insulin secretion.

143. The method of embodiment 142, wherein the GSIS is static GSIS, optionally wherein the static incubation index is greater than at or about 1, greater than at or about 2, greater than at or about 5, greater than at or about 10 or greater than at or about 20.

144. The method of any of embodiments 141-143, wherein the level of insulin secretion by the modified SC-beta cells is at least 20% of that observed for primary beta islets, optionally cadaveric islets.

145. The method of embodiment 144, wherein the level of insulin secretion by the modified SC-beta cells is at least 25%, at least 30%, at least 40%, at least 50%, at least 60%, at least 70% of that observed for primary beta islets, optionally cadaveric islets.

146. The method of any of embodiments 1-145, wherein the total insulin content of the modified SC-beta cell is greater than at or about 500 μIU Insulin per 5000 cells, greater than at or about 1000 μIU Insulin per 5000 cells, greater than at or about 2000 μIU Insulin per 5000 cells, greater than at or about 3000 μIU Insulin per 5000 cells or greater than at or about 4000 μIU Insulin per 5000 cells.

147. The method of any of embodiments 1-146, wherein the proinsulin to insulin ratio of the modified SC-beta cell is between at or about 0.02 and at or about 0.1, optionally at or about 0.03, 0.04, 0.05, 0.06, 0.07, 0.08, 0.09 and any value between any of the foregoing.

148. The method of any of embodiments 1-147, wherein the modified SC-beta cell exhibits functionality for 1 or more days following transplantation into a subject.

149. The method of any of embodiments 1-148, wherein the modified SC-beta cell exhibits functionality for more than 1 week following transplantation into a subject.

150. The method of embodiment 148 or embodiment 149, wherein the functionality is selected from the group consisting of maintaining fasting blood glucose levels, secreting insulin in response to glucose injections in vivo, and clearing glucose after a glucose injection in vivo.

151. A composition comprising a population of modified SC-beta cells produced by the method of any of embodiments 1-150.

152. A modified stem-cell derived beta cell (SC-beta cell) that has been differentiated in vitro from a pluripotent stem cell (PSC), wherein the modified SC-beta cell has (1) reduced expression of one or more of major histocompatibility complex (MHC) class I molecules and/or one or more MHC class II molecules, relative to a control or wild-type beta cell; and (2) increased expression of a tolerogenic factor, relative to the control or wild-type beta cell, and wherein the modified SC-beta cell exhibits glucose-stimulated insulin secretion (GSIS).

153. The modified SC-beta cell of embodiment 152, wherein the tolerogenic factor is expressed at a first level that is greater than at or about 10-fold, greater than at or about 20-fold, greater than at or about 30-fold, greater than at or about 40-fold, greater than at or about 50-fold, greater than at or about 60-fold, or greater than at or about 70-fold over a second level expressed by the control or wild-type beta cell.

154. A modified stem cell-derived beta cell (SC-beta cell) that has been differentiated in vitro from a pluripotent stem cell (PSC), wherein the modified SC-beta cell (1) does not express one or more major histocompatibility complex (MHC) class I molecules and/or one or more MHC class II molecules compared to background, and (2) overexpresses a tolerogenic factor at a level of greater than at or about 5-fold compared to background, and wherein the modified SC-beta cell exhibits glucose-stimulated insulin secretion (GSIS).

155. The modified SC-beta cell of embodiment 154, wherein the expression of the tolerogenic factor is by flow cytometry with an antibody directed against the tolerogenic factor and the background is determined by flow cytometry staining with an isotype control of the antibody.

156. The modified SC-beta cell of embodiment 154 or embodiment 155, wherein the tolerogenic factor is expressed at a level that is greater than at or about 10-fold, greater than at or about 20-fold, greater than at or about 30-fold, greater than at or about 40-fold, greater than at or about 50-fold, greater than at or about 60-fold, or greater than at or about 70-fold compared to background expression.

157. A modified stem cell-derived beta cell (SC-beta cell) that has been differentiated in vitro from a pluripotent stem cell (PSC), wherein the modified SC-beta cell (1) does not express one or more major histocompatibility complex (MHC) class I molecules and/or one or more MHC class II molecules and (2) overexpresses a tolerogenic factor at a first level of greater than at or about 5-fold over a second level expressed by an unmodified cell, wherein:
the unmodified cell is an unmodified PSC that does not comprise modifications to reduce one or more MHC class I molecules and/or one or more MHC class II molecules and to overexpress the tolerogenic factor or is an unmodified SC-beta cell differentiated from such unmodified PSC;
and wherein the modified SC-beta cell exhibits glucose-stimulated insulin secretion (GSIS).

158. The modified SC-beta cell of any of embodiments 152-157, wherein the tolerogenic factor is expressed by the modified SC-beta cell at greater than at or about 20,000 molecules per cell.

159. A modified stem cell-derived beta cell (SC-beta cell) that has been differentiated in vitro from a pluripotent stem cell (PSC), wherein the modified SC-beta cell (1) does not express one or more major histocompatibility complex (MHC) class I molecules and/or one or more MHC class II molecules and (2) overexpresses a tolerogenic factor, wherein the tolerogenic factor is expressed at greater than at or about 20,000 molecules per cell, and wherein the modified SC-beta cell exhibits glucose-stimulated insulin secretion (GSIS).

160. The modified SC-beta cell of any of embodiments 152-159, wherein the tolerogenic factor is expressed by the modified SC-beta cell at greater than at or about 30,000 molecules per cell, greater than at or about 50,000 molecules per cell, greater than at or about 100,000 molecules per cell, greater than at or about 200,000 molecules per cell, greater than at or about 300,000 molecules per cell, greater than at or about 400,000 molecules per cell, greater than at or about 500,000 molecules per cell, or greater than at or about 600,000 molecules per cell.

161. The modified SC-beta cell of any of embodiments 152-160, wherein the PSC is a modified PSC comprising modifications that (a) reduce expression of one or more MHC class I molecules and/or one or more MHC class II molecules, relative to a control or wild-type PSC; and (b) increase expression of a tolerogenic factor, relative to the control or wild-type PSC.

162. The modified SC-beta cell of embodiment 161, wherein the control or wild-type PSC is an unmodified PSC that does not comprise the modifications.

163. The modified SC-beta cell of embodiment 152 and 159-162embodiment, wherein the modified SC-beta cell expresses the tolerogenic factor at a first level that is greater than at or about 5-fold over a second level expressed by the unmodified PSC or the unmodified SC-beta cell differentiated from the unmodified PSC.

164. The modified SC-beta cell of embodiment 163, wherein the tolerogenic factor is expressed at a first level that is greater than at or about 10-fold, greater than at or about 20-fold, greater than at or about 30-fold, greater than at or about 40-fold, greater than at or about 50-fold, greater than at or about 60-fold, or greater than at or about 70-fold over a second level expressed by the unmodified PSC or an unmodified SC-beta differentiated from the unmodified PSC.

165. A modified stem-cell derived beta cell (SC-beta cell) that has been differentiated in vitro from a modified pluripotent stem cell (PSC), wherein the modified PSC comprises modifications that (a) reduce expression of one or more major histocompatibility complex (MHC) class I molecules and/or one or more MHC class II molecules, relative to a control or wild-type PSC; and (b) increase expression of a tolerogenic factor, relative to the control or wild-type PSC, and wherein the modified SC-beta cell exhibits glucose-stimulated insulin secretion (GSIS).

166. The modified SC-beta cell of embodiment 165, wherein the control or wild-type PSC is an unmodified PSC that does not comprise the modifications.

167. The modified SC-beta cell of any of embodiments 161-166, wherein the modified PSC expresses the tolerogenic factor at a first level that is greater than at or about 5-fold over a second level expressed by the unmodified PSC that does not comprise the modifications, optionally wherein the tolerogenic factor is expressed at a first level that is greater than at or about 10-fold, greater than at or about 20-fold, greater than at or about 30-fold, greater than at or about 40-fold, greater than at or about 50-fold, greater than at or about 60-fold, or greater than at or about 70-fold over the second level expressed by the unmodified PSC.

168. The modified SC-beta cell of any of embodiments 161-167, wherein the modified SC-beta cell comprises modifications that (1) reduce expression of MHC class I and/or or MHC class II molecules, relative to the unmodified PSC or an unmodified SC-beta differentiated from the unmodified PSC; and (2) increase expression of a tolerogenic factor, relative to the unmodified PSC or the unmodified SC-beta differentiated from the unmodified PSC.

169. The modified SC-beta cell of embodiment 168, wherein the modified SC-beta cell expresses the tolerogenic factor at a first level that is greater than at or about 5-fold over a second level expressed by the unmodified PSC or the unmodified SC-beta cell differentiated from the unmodified PSC.

170. The modified SC-beta cell of embodiment 169, wherein the tolerogenic factor is expressed at a first level that is greater than at or about 10-fold, greater than at or about 20-fold, greater than at or about 30-fold, greater than at or about 40-fold, greater than at or about 50-fold, greater than at or about 60-fold, or greater than at or about 70-fold over a second level expressed by the unmodified PSC or the unmodified SC-beta cell differentiated from the unmodified PSC.

171. The modified SC-beta of any of embodiments 161-170, wherein the tolerogenic factor is expressed by the modified PSC at greater than at or about 20,000 molecules per cell.

172. A modified stem cell-derived beta cell (SC-beta cell) that has been differentiated in vitro from a modified pluripotent stem cell (PSC), wherein the modified PSC comprises modifications such that the modified PSC (a) does not express one or more major histocompatibility complex (MHC) class I molecules and/or or one or more MHC class II molecules; and (b) expresses a tolerogenic factor at greater than at or about 20,000 molecules per cell, and wherein the modified SC-beta cell exhibits glucose-stimulated insulin secretion (GSIS).

173. The modified SC-beta cell of embodiment 171 or 172, wherein the tolerogenic factor is expressed by the modified PSC at greater than at or about 30,000 molecules per cell, greater than at or about 50,000 molecules per cell, greater than at or about 100,000 molecules per cell, greater than at or about 200,000 molecules per cell, greater than at or about 300,000 molecules per cell, greater than at or about 400,000 molecules per cell, greater than at or about 500,000 molecules per cell, or greater than at or about 600,000 molecules per cell.

174. The modified SC-beta cell of any of embodiments 161-173, wherein the modified SC-beta cell does not express MHC class I or MHC class II molecules and expresses the tolerogenic factor at greater than at or about 20,000 molecules per cell.

175. The modified SC-beta cell of embodiment 174, wherein the tolerogenic factor is expressed by the modified SC-beta cell at greater than at or about 30,000 molecules per cell, greater than at or about 50,000 molecules per cell, greater than at or about 100,000 molecules per cell, greater than at or about 200,000 molecules per cell, greater than at or about 300,000 molecules per cell, greater than at or about 400,000 molecules per cell, greater than at or about 500,000 molecules per cell, or greater than at or about 600,000 molecules per cell.

176. The modified SC-beta cell of any of embodiments 152-175, wherein the tolerogenic factor is selected from the group consisting of CD47, CD27, CD200, HLA-C, HLA-E, HLA-E heavy chain, HLA-G, PD-L1, IDO1, CTLA4-Ig, Cl-Inhibitor, IL-10, IL-35, FASL, CCL21, MFGE8, and SERPINB9, and any combination thereof.

177. The modified SC-beta cell of any of embodiments 152-176, wherein the tolerogenic factor comprises CD47.

178. The modified SC-beta cell of any of embodiments 152-176, wherein the tolerogenic factor comprises PD-L1.

179. The modified SC-beta cell of any of embodiments 152-176, wherein the tolerogenic factor comprises HLA-E.

180. The modified SC-beta cell of any of embodiments 152-176, wherein the tolerogenic factor comprises HLA-G.

181. The modified SC-beta cell of any of embodiments 161-180, wherein expression of one or more MHC class I molecules and one or more MHC class II molecules is reduced in the modified PSC.

182. The modified SC-beta cell of any of embodiments 161-181, wherein the modifications in (a) reduce protein expression of one or more MHC class I molecules.

183. The modified SC-beta cell of any of embodiment 161-182, wherein the modifications in (a) reduce cell surface expression of the one or more MHC class I molecules.

184. The modified SC-beta cell of any of embodiments 161-183, wherein the modifications in (a) reduce a function of the one or more MHC class I molecules, optionally wherein the function is antigen presentation.

185. The modified SC-beta cell of any of embodiments 161-184, wherein the one or more MHC class I molecules is one or more human leukocyte antigen (HLA) class I molecules.

186. The modified SC-beta cell of any of embodiments 161-185, wherein the one or more MHC HLA class I molecules is selected from the group consisting of HLA-A, HLA-B, and HLA-C

187. The modified SC-beta cell of any of embodiments 161-186, wherein the modification that reduces expression of the one or more MHC class I molecules reduces expression of B2M.

188. The modified SC-beta cell of any of embodiments 161-187, wherein the modification that reduces expression of the one or more MHC class I molecules reduces mRNA expression of the B2M gene.

189. The modified SC-beta cell of any of embodiments 161-188, wherein the modification that reduces expression of the one or more MHC class I molecules reduces protein expression of B2M.

190. The modified SC-beta cell of any of embodiments 161-189, wherein the modification that reduces expression of the one or more MHC class I molecules comprises:
inactivation or disruption of one allele of the B2M gene;
inactivation or disruption of both alleles of the B2M gene; or
inactivation or disruption of all B2M coding alleles in the cell.

191. The modified SC-beta cell of embodiment 190, wherein the inactivation or disruption comprises an indel in the B2M gene.

192. The modified SC-beta cell of embodiment 190 or embodiment 191, wherein the inactivation or disruption is a frameshift mutation or a deletion of a contiguous stretch of genomic DNA of the B2M gene.

193. The modified SC-beta cell of any of embodiments 161-192, wherein the modifications in (a) reduce protein expression of the one or more MHC class II molecules.

194. The modified SC-beta cell of any of embodiments 161-193, wherein the modifications in (a) reduce cell surface expression of the one or more MHC class II molecules.

195. The modified SC-beta cell of any of embodiments 161-194, wherein the modifications in (a) reduce a function of the one or more MHC class II molecules, optionally wherein the function is antigen presentation.

196. The modified SC-beta cell of any of embodiments 161-195, wherein the one or more MHC class II molecules is one or more human leukocyte antigen (HLA) class II molecules.

197. The modified SC-beta cell of any of embodiments 161-196, wherein the one or more MHC HLA class II molecules is selected from the group consisting of HLA-DP, HLA-DQ, and/or HLA-DR.

198. The modified SC-beta cell of any of embodiments 161-197, wherein the modification that reduces expression of the one or more MHC class II molecules comprises reduced expression of CIITA.

199. The modified SC-beta cell of any of embodiments 161-198, wherein the modification that reduces expression of the one or more MHC class II molecules reduces mRNA expression of the CIITA gene.

200. The modified SC-beta cell of any of embodiments 161-199, wherein the modification that reduces expression of the one or more MHC class II molecules reduces protein expression of CIITA.

201. The modified SC-beta cell of any of embodiments 161-200, wherein the modification that reduces expression of the one or more MHC class II molecules comprises:
inactivation or disruption of one allele of the CIITA gene;
inactivation or disruption of both alleles of the CIITA gene; or
inactivation or disruption of all CIITA coding alleles in the cell.

202. The modified SC-beta cell of embodiment 201, wherein the inactivation or disruption comprises an indel in the CIITA gene.

203. The modified SC-beta cell of embodiment 201 or embodiment 202, wherein the inactivation or disruption is a frameshift mutation or a deletion of a contiguous stretch of genomic DNA of the CIITA gene.

204. The modified SC-beta cell of any of embodiments 161-203, wherein the modified PSC comprises a modification that reduces expression of CD142.

205. The modified SC-beta cell of embodiment 204, wherein the modification reduces mRNA expression of the CD142 gene.

206. The modified SC-beta cell of embodiment 204 or embodiment 205, wherein the modification reduces protein expression of CD142.

207. The modified SC-beta cell of any of embodiments 204-206, wherein the modification that reduces expression of CD142 comprises:
inactivation or disruption of one allele of the CD142 gene;
inactivation or disruption of both alleles of the CD142 gene; or
inactivation or disruption of all CD142 coding alleles in the cell.

208. The modified SC-beta cell of embodiment 207, wherein the inactivation or disruption comprises an indel in the CD142 gene.

209. The modified SC-beta cell of embodiment 207 or embodiment 208, wherein the inactivation or disruption is a frameshift mutation or a deletion of a contiguous stretch of genomic DNA of the CD142 gene.

210. The modified SC-beta cell of any of embodiments 161-209, wherein the modification to increase expression of the tolerogenic factor comprises an exogenous polynucleotide encoding the tolerogenic factor.

211. The modified SC-beta cell of embodiment 210, wherein the exogenous polynucleotide encoding the tolerogenic factor is integrated into the genome of the modified PSC.

212. The modified SC-beta cell of embodiment 211, wherein the exogenous polynucleotide encoding the tolerogenic factor is integrated by non-targeted insertion into the genome of the modified PSC.

213. The modified SC-beta cell of embodiment 212, wherein the exogenous polynucleotide encoding the tolerogenic factor is integrated by targeted insertion into a target genomic locus of the modified PSC.

214. A modified stem cell derived beta cell (SC-beta cell) that has been differentiated in vitro from a modified pluripotent stem cell (PSC), wherein the modified SC-beta cell comprises knock out of the B2M gene, knock out of the CIITA gene, and an exogenous polynucleotide encoding exogenous CD47 protein, relative to a control or wild-type beta cell.

215. The modified SC-beta cell of embodiment 214, that has the phenotype B2M^{indel/indel}. CIITA^{indel/indel}, CD47tg.

216. A modified stem cell derived beta cell (SC-beta cell) that has been differentiated in vitro from a modified pluripotent stem cell (PSC), wherein the modified SC-beta cell comprises knock out of the B2M gene, knock out of the CIITA gene, an exogenous polynucleotide encoding CD47 protein, and an exogenous polynucleotide encoding a suicide gene, relative to a control or wild-type beta cell.

217. The modified SC-beta cell of embodiment 216, wherein the modified SC-beta cell has the phenotype B2M^{indel/indel}; CIITA^{indel/indel}; CD47tg; suicide genetg.

218. The modified SC-beta cell of any of embodiments 214-217, wherein the exogenous polynucleotide encoding CD47 is integrated by non-targeted insertion into the genome of the modified PSC.

219. The modified SC-beta cell of any of embodiments 214-218, wherein the exogenous polynucleotide encoding CD47 is integrated by targeted insertion into a target genomic locus of the cell.

220. The modified SC-beta cell of any of embodiment 161-213, 214,215, 218 and 219, wherein the modified PSC comprises an exogenous polynucleotide encoding a suicide gene.

221. The modified SC-beta cell of any of embodiments 216-220, wherein the suicide gene is selected from the group consisting of cytosine deaminase (CyD), herpesvirus thymidine kinase (HSV-Tk), an inducible caspase (iCaspase9), and rapamycin-activated caspase 9 (rapaCasp9).

222. The modified SC-beta cell of embodiment 220 and 221, wherein the suicide gene and the tolerogenic factor are expressed from a bicistronic cassette integrated into the genome of the modified PSC.

223. The modified SC-beta cell of embodiment 216,217 and 221, wherein the suicide gene and CD47 are expressed from a bicistronic cassette integrated into the genome of the modified PSC.

224. The modified SC-beta cell of embodiment 222 or embodiment 223, wherein the bicistronic cassette is integrated by non-targeted insertion into the genome of the modified PSC.

225. The modified SC-beta cell of embodiment 222 or embodiment 223, wherein the bicistronic cassette is integrated by targeted insertion into a target genomic locus of the modified PSC.

226. The modified SC-beta cell of embodiment 213, embodiment 219, or embodiment 225, wherein the target genomic locus is a safe harbor locus, a B2M gene locus, a CIITA gene locus, or a CD142 gene locus.

227. The modified SC-beta cell of embodiment 226, wherein the safe harbor locus is selected from the group consisting of: a CCR5 gene locus, a CXCR4 gene locus, a PPP1R12C (also known as AAVS1) gene, an albumin gene locus, a SHS231 locus, a CLYBL gene locus, and a ROSA26 gene locus.

228. The modified SC-beta cell of any of embodiments 161-227, wherein the modified PSC comprises a modification that increases expression of one or more complement inhibitors selected from the group consisting of CD46, CD59, CD55, and CD35 relative to the control or wild-type PSC.

229. The modified SC-beta cell of embodiment 228, wherein the modification to increase expression of one or more complement inhibitors comprises at least one exogenous polynucleotide encoding one or more complement inhibitors selected from the group consisting of an exogenous polynucleotide encoding CD46, an exogenous polynucleotide encoding CD59, an exogenous polynucleotide encoding CD55, and an exogenous polynucleotide encoding CD35.

230. The modified SC-beta cell of embodiment 228 or embodiment 229, wherein the one or more complement inhibitors is CD46 and CD59.

231. The modified SC-beta cell of embodiment 228 or embodiment 229, wherein the one or more complement inhibitor is CD46, CD59 and CD55.

232. The modified SC-beta cell of any of embodiments 228-231, wherein the at least one exogenous polynucleotide encoding the one or more complement inhibitors is integrated by non-targeted insertion into the genome of the modified PSC.

233. The modified SC-beta cell of any of embodiments 228-231, wherein the at least one exogenous polynucleotide encoding the one or more complement inhibitors is integrated by targeted insertion into a target genomic locus of the cell.

234. The modified SC-beta cell of embodiment 233, wherein the target genomic locus is a safe harbor locus, a B2M gene locus, a CIITA gene locus, or a CD142 gene locus.

235. The modified SC-beta cell of embodiment 234, wherein the safe harbor locus is selected from the group consisting of: a CCR5 gene locus, a CXCR4 gene locus, a PPP1R12C (also known as AAVS1) gene, an albumin gene locus, a SHS231 locus, a CLYBL gene locus, and a ROSA26 gene locus.

236. The modified SC-beta cell of any of embodiments 152-235, wherein the expression of the one or more MHC class I molecules and the one or more MHC class II molecules is reduced in the modified SC-beta cell.

237. The modified SC-beta cell of any of embodiments 152-236, wherein the modifications in (1) reduce protein expression of the one or more MHC class I molecules in the modified SC-beta cell.

238. The modified SC-beta cell of any of embodiment 152-237, wherein the modifications in (1) reduce cell surface expression of the one or more MHC class I molecules in the modified SC-beta cell

239. The modified SC-beta cell of any of embodiments 152-238, wherein the modifications in (1) reduce a function of the one or more MHC class I molecules in the modified SC-beta cell, optionally wherein the function is antigen presentation.

240. The modified SC-beta cell of any of embodiments 152-239, wherein the modification that reduces expression of the one or more MHC class I molecules in the modified SC-beta cell comprises reduced expression of B2M.

241. The modified SC-beta cell of any of embodiments 152-240, wherein the modification that reduces expression of the one or more MHC class I molecules in the modified SC-beta cell reduces mRNA expression of the B2M gene.

242. The modified SC-beta cell of any of embodiments 152-241, wherein the modification that reduces expression of the one or more MHC class I molecules in the modified SC-beta cell reduces protein expression of B2M.

243. The modified SC-beta cell of any of embodiments 152-242, wherein the modification that reduces expression of the one or more MHC class I molecules in the modified SC-beta cell comprises:
inactivation or disruption of one allele of the B2M gene;
inactivation or disruption of both alleles of the B2M gene; or
inactivation or disruption of all B2M coding alleles in the cell.

244. The modified SC-beta cell of embodiment 243, wherein the inactivation or disruption comprises an indel in the B2M gene.

245. The modified SC-beta cell of embodiment 243 and embodiment 244, wherein the inactivation or disruption is a frameshift mutation or a deletion of a contiguous stretch of genomic DNA of the B2M gene.

246. The modified SC-beta cell of any of embodiments 152-245, wherein the modifications in (1) reduce protein expression of the one or more MHC class II molecules.

247. The modified SC-beta cell of any of embodiments 152-246, wherein the modifications in (1) reduce cell surface expression of the one or more MHC class II molecules.

248. The modified SC-beta cell of any of embodiments 152-247, wherein the modifications in (1) reduce a function of the one or more MHC class II molecules, optionally wherein the function is antigen presentation.

249. The modified SC-beta cell of any of embodiments 152-248, wherein the modification that reduces expression of the one or more MHC class II molecules in the modified SC-beta cell comprises reduced expression of CIITA.

250. The modified SC-beta cell of any of embodiments 152-249, wherein the modification that reduces expression of the one or more MHC class II molecules in the modified SC-beta cell reduces mRNA expression of the CIITA gene.

251. The modified SC-beta cell of any of embodiments 152-250, wherein the modification that reduces expression of the one or more MHC class II molecules in the modified SC-beta cell reduces protein expression of CIITA.

252. The modified SC-beta cell of any of embodiments 152-251, wherein the modification that reduces expression of the one or more MHC class II molecules in the modified SC-beta cell comprises:
inactivation or disruption of one allele of the CIITA gene;
inactivation or disruption of both alleles of the CIITA gene; or
inactivation or disruption of all CIITA coding alleles in the cell.

253. The modified SC-beta cell of embodiment 252, wherein the inactivation or disruption comprises an indel in the CIITA gene.

254. The modified SC-beta cell of embodiment 252 or embodiment 253, wherein the inactivation or disruption is a frameshift mutation or a deletion of a contiguous stretch of genomic DNA of the CIITA gene.

255. The modified SC-beta cell of any of embodiments 152-254, wherein the modified SC-beta cell comprises a modification that reduces expression of CD142, relative to a control or wild-type beta cell.

256. The modified SC-beta cell of embodiment 255, wherein the modification reduces mRNA expression of the CD142 gene.

257. The modified SC-beta cell of embodiment 255 or embodiment 256, wherein the modification reduces protein expression of CD142.

258. The modified SC-beta cell of any of embodiments 152-257, wherein the modifications that reduce expression of CD142 in the modified SC-beta cell comprises:
inactivation or disruption of one allele of the CD142 gene;
inactivation or disruption of both alleles of the CD142 gene; or
inactivation or disruption of all CD142 coding alleles in the cell.

259. The modified SC-beta cell of embodiment 258, wherein the inactivation or disruption comprises an indel in the CD142 gene.

260. The modified SC-beta cell of embodiment 258 or embodiment 249, wherein the inactivation or disruption comprises a frameshift mutation or a deletion of a contiguous stretch of genomic DNA of the CD142 gene.

261. The modified SC-beta cell of any of embodiments 152-260, wherein the modifiecation to increase expression of the tolerogenic factor in the modified SC-beta cell comprises an exogenous polynucleotide encoding the tolerogenic factor.

262. The modified SC-beta cell of embodiment 261, wherein the exogenous polynucleotide encoding the tolerogenic factor is integrated into the genome of the modified SC-beta cell.

263. The modified SC-beta cell of embodiment 262, wherein the exogenous polynucleotide is integrated into a non-target locus in the genome of the modified SC-beta cell.

264. The modified SC-beta cell of embodiment 262, wherein the exogenous polynucleotide is integrated into a target genomic locus of the modified SC-beta cell.

265. The modified SC-beta cell of any of embodiment 152-264, wherein the modified SC-beta cell further comprises a modification for expression of an exogenous suicide gene selected from the group consisting of cytosine deaminase (CyD), herpesvirus thymidine kinase (HSV-Tk), an inducible caspase (iCaspase9), and rapamycin-activated caspase 9 (rapaCasp9).

266. The modified SC-beta cell of embodiment 265, wherein the suicide gene and the tolerogenic factor are expressed from a bicistronic cassette integrated into the genome of the modified SC-beta cell.

267. The modified SC-beta cell of embodiment 266, wherein the bicistronic cassette is integrated at a non-target locus in the genome of the modified SC-beta cell.

268. The modified SC-beta cell of embodiment 266, wherein the bicistronic cassette is integrated into a target genomic locus of the cell.

269. The modified SC-beta cell of embodiment 264 or embodiment 268, wherein the target genomic locus is a safe harbor locus, a B2M gene locus, a CIITA gene locus, or a CD142 gene locus.

270. The modified SC-beta cell of embodiment 269, wherein the safe harbor locus is selected from the group consisting of: a CCR5 gene locus, a CXCR4 gene locus, a PPPIR12C (also known as AAVS1) gene, an albumin gene locus, a SHS231 locus, a CLYBL gene locus, and a ROSA26 gene locus.

271. The modified SC-beta cell of any of embodiments 152-270, wherein the modified SC-beta cell comprises a modification that increases expression of one or more complement inhibitors selected from the group consisting of CD46, CD59, CD55, and CD35 relative to a control or wild-type beta cell.

272. The modified SC-beta cell of embodiment 271, wherein the modification to increase expression of the one or more complement inhibitors in the modified SC-beta cell comprises at least one exogenous polynucleotide encoding one or more complement inhibitors selected from the group consisting of an exogenous polynucleotide encoding CD46, an exogenous polynucleotide encoding CD59, an exogenous polynucleotide encoding CD55, and an exogenous polynucleotide encoding CD35.

273. The modified SC-beta cell of embodiment 271 or embodiment 272, wherein the one or more complement inhibitors is CD46 and CD59.

274. The modified SC-beta cell of embodiment 271 or embodiment 272, wherein the one or more complement inhibitor is CD46, CD59 and CD55.

275. The modified SC-beta cell of any of embodiments 152-274, wherein the tolerogenic factor is CD47 and the modified SC-beta cell expresses CD47 at a first level that is greater than at or about 5-fold over a second level expressed by the control or wild-type beta cell.

276. The modified SC-beta cell of embodiment 275, wherein CD47 is expressed at a first level that is greater than at or about 10-fold, greater than at or about 20-fold, greater than at or about 30-fold, greater than at or about 40-fold, greater than at or about 50-fold, greater than at or about 60-fold, or greater than at or about 70-fold over a second level expressed by the control or wild-type beta cell.

277. The modified SC-beta cell of any of embodiments 152-277, wherein the tolerogenic factor is CD47 and CD47 is expressed by the modified SC-beta cell at greater than at or about 20,000 molecules per cell.

278. The modified SC-beta cell of embodiment 277, wherein CD47 is expressed by the modified SC-beta cell at greater than at or about 30,000 molecules per cell, greater than at or about 50,000 molecules per cell, greater than at or about 100,000 molecules per cell, greater than at or about 200,000 molecules per cell, greater than at or about 300,000 molecules per cell, greater than at or about 400,000 molecules per cell, greater than at or about 500,000 molecules per cell, or greater than at or about 600,000 molecules per cell.

279. The modified SC-beta cell of any of embodiments 152-278, wherein the control or wild-type beta cell is an unmodified SC-beta cell differentiated from an unmodified PSC not comprising modifications that reduce expression of the one or more MHC class I molecules and/or the one or more MHC class II molecules and that increase expression of the tolerogenic factor or is a wild-type primary beta cell.

280. The modified SC-beta of any of embodiments 152-279, wherein the modified SC-beta cell expresses at least one beta cell marker, optionally wherein the beta cell marker is selected from the group consisting of INS, CHGA, NKX2-2, PDX1, NKX6-1, MAFB, GCK and GLUT1.

281. The modified SC-beta cell of any of embodiments 152-280, wherein the modified SC-beta cell exhibits one or more functions of a wild-type or control beta cell, optionally wherein the one or more functions is selected from the group consisting of in vitro glucose-stimulated insulin secretion (GSIS), glucose metabolism, maintaining fasting blood glucose levels, secreting insulin in response to glucose injections in vivo, and clearing glucose after a glucose injection in vivo.

282. The modified SC-beta cell of any of embodiments 152-281, wherein the GSIS is measured in a perfusion GSIS assay.

283. The modified SC-beta cell of any of embodiments 152-282, wherein the GSIS is dynamic GSIS comprising first and second phase dynamic insulin secretion.

284. The modified SC-beta cell of any of embodiments 152-283, wherein the GSIS is static GSIS, optionally wherein the static stimulation index is greater than at or about 1, greater than at or about 1.5, greater than at or about 2, greater than at or about 5, greater than at or about 10, greater than at or about 15, or greater than at or about 20.

285. The modified SC-beta cell of any of embodiments 152-284, wherein the level of insulin secretion by the modified SC-beta cells is at least 20% of that observed for primary beta islets, optionally cadaveric islets.

286. The modified SC-beta cell of any of embodiments 152-285, wherein the level of insulin secretion by the modified SC-beta cells is at least 25%, at least 30%, at least 40%, at least 50%, at least 60%, at least 70% of that observed for primary beta islets, optionally cadaveric islets.

287. The modified SC-beta cell of any of embodiments 152-286, wherein the total insulin content of the modified SC-beta is greater than at or about 500 μIU Insulin per 5000 cells, greater than at or about 1000 μIU Insulin per 5000 cells, greater than at or about 2000 μIU Insulin per 5000 cells, greater than at or about 3000 μIU Insulin per 5000 cells or greater than at or about 4000 μIU Insulin per 5000 cells.

288. The modified SC-beta cell of any of embodiments 152-287, wherein the proinsulin to insulin ratio of the modified SC-beta is between at or about 0.02 and at or about 0.1, optionally at or about 0.03, 0.04, 0.05, 0.06, 0.07, 0.08, 0.09, and any value between any of the foregoing.

289. The modified SC-beta cell of any of embodiments 152-288, wherein the modified SC-beta cell exhibits functionality for 1 or more days following transplantation into a subject.

290. The modified SC-beta cell of any of embodiments 152-289, wherein the modified SC-beta cell exhibits functionality for more than 1 week following transplantation into a subject.

291. The modified SC-beta cell of embodiment 289 or embodiment 290, wherein the functionality is selected from the group consisting of maintaining fasting blood glucose levels, secreting insulin in response to glucose injections in vivo, and clearing glucose after a glucose injection in vivo.

292. A composition comprising a modified SC-beta cell of any of embodiments 152-291.

293. A composition comprising a population of modified SC-beta cells of any of embodiments 152-291.

294. The composition of embodiment 151 or embodiment 293, wherein, among the cells in the population, the level of the reduced expression of MHC class I molecules and/or MHC class II molecules and/or the level of the increased expression of the tolerogenic factor is retained or is similar compared to the modified PSC in at least at or about 50%, at least at or about 60%, at least at or about 70%, at least at or about 80%, or at least at or about 90% of the cells in the population.

295. The composition of embodiment 151, embodiment 293 or embodiment 294, wherein at least at or about 50%, at least at or about 60%, at least at or about 70%, at least at or about 80%, or at least at or about 90% of the cells in the population are reduced for expression of MHC class I molecules or for B2M.

296. The composition of any of embodiments 151 or 293-295, wherein at least at or about 50%, at least at or about 60%, at least at or about 70%, at least at or about 80%, or at least at or about 90% of the cells in the population are reduced for expression of MHC class II molecules or for CIITA.

297. The composition of any of embodiments 151 or 293-296, wherein at least at or about 50%, at least at or about 60%, at least at or about 70%, at least at or about 80%, or at least at or about 90% of the cells in the population express the tolerogenic factor at a first level that is greater than at or about 5-fold, greater than at or about 10-fold, greater than at or about 20-fold, greater than at or about 30-fold, greater than at or about 40-fold, greater than at or about 50-fold, greater than at or about 60-fold, or greater than at or about 70-fold over a second level expressed by the control or wild-type beta cell, optionally wherein the control or wild-type beta cell is a wild-type primary beta cell.

298. The composition of any of embodiments 151 or 293-297, wherein at least at or about 50%, at least at or about 60%, at least at or about 70%, at least at or about 80%, or at least at or about 90% of the cells in the population express the tolerogenic factor at a first level that is greater than at or about 5-fold, greater than at or about 10-fold, greater than at or about 20-fold, greater than at or about 30-fold, greater than at or about 40-fold, greater than at or about 50-fold, greater than at or about 60-fold, or greater than at or about 70-fold over a second level expressed by an unmodified PSC not comprising the modifications or an unmodified SC-beta cell differentiated from the unmodified PSC.

299. The composition of any of embodiments 151 or 293-298, wherein at least at or about 50%, at least at or about 60%, at least at or about 70%, at least at or about 80%, or at least at or about 90% of the cells in the population expresses the tolerogenic factor at greater than at or about 20,000 molecules per cell, at greater than at or about 30,000 molecules per cell, greater than at or about 50,000 molecules per cell, greater than at or about 100,000 molecules per cell, greater than at or about 200,000 molecules per cell, greater than at or about 300,000 molecules per cell, greater than at or about 400,000 molecules per cell, greater than at or about 500,000 molecules per cell, or greater than at or about 600,000 molecules per cell.

300. The composition of any of embodiments 151 or 293-299, wherein at least at or about 50%, at least at or about 60%, at least at or about 70%, at least at or about 80%, or at least at or about 90% of the cells in the population are reduced for expression of CD142.

301. The composition of any of embodiments 151 and 292-300 comprising a pharmaceutically acceptable excipient.

302. The composition of any of embodiments 151 and 292-301 comprising a cryoprotectant.

303. A method of treating diabetes in a subject, the method comprising administering the modified SC-beta cells of any of embodiments 152-291 or the composition of any of embodiments 151 and 292-302 to a subject in need thereof.

304. The method of embodiment 303, wherein the diabetes is type I diabetes.

305. The method of embodiment 304, wherein the diabetes is type II diabetes.

306. The method of any of embodiments 303-305, wherein the modified SC-beta cells improve glucose tolerance in the subject.

307. A method for improving glucose tolerance in a subject, the method comprising administering the modified SC-beta cells of any of embodiments 152-291 or the composition of any of embodiments 151 and 292-302 to a subject in need thereof.

308. The method of any of embodiments 303-307, wherein the subject is a diabetic patient.

309. The method of embodiment 308, wherein the diabetic patient has type I diabetes or type II diabetes.

310. The method of any of embodiments 306-309, wherein glucose tolerance is improved relative to the subject's glucose tolerance prior to administration of the modified SC-beta cells.

311. The method of any of embodiments 303-310, wherein administration of the modified SC-beta cells reduces exogenous insulin usage in the subject.

312. The method of any of embodiments 306-311, wherein glucose tolerance is improved as measured by HbA1c levels.

313. The method of any of embodiments 303-312, wherein the subject is fasting.

314. The method of any one of embodiments 303-313, wherein administration of the modified SC-beta cells improves insulin secretion in the subject.

315. The method of embodiment 314, wherein insulin secretion is improved relative to the subject's insulin secretion prior to administration of the modified SC-beta cells.

316. The method of any of embodiments 303-315, further comprising administering one or more immunosuppressive agents to the subject.

317. The method of any of embodiments 303-315, wherein the subject has been administered one or more immunosuppressive agents.

318. The method of embodiment 316 or 317, wherein the one or more immunosuppressive agents are a small molecule or an antibody.

319. The method of any of embodiments 316-318, wherein the one or more immunosuppressive agents are selected from the group consisting of cyclosporine, azathioprine, mycophenolic acid, mycophenolate mofetil, a corticosteroids, prednisone, methotrexate, gold salts, sulfasalazine, antimalarials, brequinar, leflunomide, mizoribine, 15-deoxyspergualine, 6-mercaptopurine, cyclophosphamide, rapamycin, tacrolimus (FK-506), OKT3, anti-thymocyte globulin, thymopentin (thymosin-α), and an immunosuppressive antibody.

320. The method of any of embodiments 316-319, wherein the one or more immunosuppressive agents comprise cyclosporine.

321. The method of any of embodiments 316-319, wherein the one or more immunosuppressive agents comprise mycophenolate mofetil.

322. The method of any of embodiments 316-319, wherein the one or more immunosuppressive agents comprise a corticosteroid.

323. The method of any of embodiments 316-319, wherein the one or more immunosuppressive agents comprise cyclophosphamide.

324. The method of any of embodiments 316-319, wherein the one or more immunosuppressive agents comprise rapamycin.

325. The method of any of embodiments 316-319, wherein the one or more immunosuppressive agents comprise tacrolimus (FK-506).

326. The method of any of embodiments 316-319, wherein the one or more immunosuppressive agents comprise anti-thymocyte globulin.

327. The method of any of embodiments 316-319, wherein the one or more immunosuppressive agents are one or more immunomodulatory agents.

328. The method of embodiment 327, wherein the one or more immunomodulatory agents are a small molecule or an antibody.

329. The method of embodiment 318 or embodiment 328, wherein the antibody binds to one or more of receptors or ligands selected from the group consisting of p75 of the IL-2 receptor, MHC, CD2, CD3, CD4, CD7, CD28, B7, CD40, CD45, IFN-gamma, TNF-alpha, IL-4, IL-5, IL-6R, IL-6, IGF, IGFR1, IL-7, IL-8, IL-10, CD11a, CD58, and antibodies binding to any of their ligands.

330. The method of any of embodiments 362-375, wherein the one or more immunosuppressive agents are or have been administered to the subject prior to administration of the modified SC-beta cells.

331. The method of any of embodiments 316-330, wherein the one or more immunosuppressive agents are or have been administered to the subject at least 1, 2, 3, 4, 5, 6, 7, 8, 9, 10, 11, 12, 13, or 14 days prior to administration of the modified SC-beta cell s.

332. The method of any of embodiments 316-330, wherein the one or more immunosuppressive agents are or have been administered to the subject at least 1 week, 2 weeks, 3 weeks, 4 weeks, 5 weeks, 6 weeks, 7 weeks, 8 weeks, 9 weeks, 10 weeks or more prior to administration of the s.

333. The method of any of embodiments 316-330, wherein the one or more immunosuppressive agents are or have been administered to the subject at least 1, 2, 3, 4, 5, 6, 7, 8, 9, 10, 11, 12, 13, or 14 days after administration of the modified SC-beta cells.

334. The method of any of embodiments 316-330, wherein the one or more immunosuppressive agents are or have been administered to the subject at least 1 week, 2 weeks, 3 weeks, 4 weeks, 5 weeks, 6 weeks, 7 weeks, 8 weeks, 9 weeks, 10 weeks, or more, after administration of the modified SC-beta cells.

335. The method of any of embodiments 316-330, wherein the one or more immunosuppressive agents are or have been administered to the subject on the same day as the first administration of the modified SC-beta cells.

336. The method of any of embodiments 316-330, wherein the one or more immunosuppressive agents are or have been administered to the subject after administration of the modified SC-beta cells.

337. The method of any of embodiments 316-330, wherein the one or more immunosuppressive agents are or have been administered to the subject after administration of a first and/or second administration of the modified SC-beta cells.

338. The method of any of embodiments 316-330, wherein the one or more immunosuppressive agents are or have been administered to the subject prior to administration of a first and/or second administration of the modified SC-beta cells.

339. The method of any of embodiments 316-330, wherein the one or more immunosuppressive agents are or have been administered to the subject at least 1, 2, 3, 4, 5, 6, 7, 8, 9, 10, 11, 12, 13, or 14 days prior to administration of a first and/or second administration of the modified SC-beta cells.

340. The method of any of embodiments 316-330, wherein the one or more immunosuppressive agents are or have been administered to the subject at least 1 week, 2 weeks, 3 weeks, 4 weeks, 5 weeks, 6 weeks, 7 weeks, 8 weeks, 9 weeks, 10 weeks or more prior to administration of a first and/or second administration of the modified SC-beta cells.

341. The method of any of embodiments 316-330, wherein the one or more immunosuppressive agents are or have been administered to the subject at least 1, 2, 3, 4, 5, 6, 7, 8, 9, 10, 11, 12, 13, or 14 days after administration of a first and/or second administration of the modified SC-beta cells.

342. The method of any of embodiments 316-330, wherein the one or more immunosuppressive agents are or have been administered to the subject at least 1 week, 2 weeks, 3 weeks, 4 weeks, 5 weeks, 6 weeks, 7 weeks, 8 weeks, 9 weeks, 10 weeks, or more, after administration of a first and/or second administration of the modified SC-beta cells.

343. The method of any of embodiments 316-342, wherein the one or more immunosuppressive agents are administered at a lower dosage compared to the dosage of one or more immunosuppressive agents administered to reduce immune rejection of immunogenic cells that do not comprise the modifications of the modified SC-beta cells.

344. The method of any of embodiments 316-343, wherein the modified SC-beta cell is capable of controlled killing of the modified SC-beta cell.

345. The method of any of embodiments 316-344, wherein the modified SC-beta cell comprises a suicide gene or a suicide switch.

346. The method of embodiment 345, wherein the suicide gene or the suicide switch induces controlled cell death in the presence of a drug or prodrug, or upon activation by a selective exogenous compound.

347. The method of embodiment 345 or embodiment 346, wherein the suicide gene or the suicide switch is an inducible protein capable of inducing apoptosis of the modified SC-beta cell.

348. The method of embodiment 347, wherein the inducible protein capable of inducing apoptosis of the modified SC-beta cell is a caspase protein.

349. The method of embodiment 348, wherein the caspase protein is caspase 9.

350. The method of any of embodiments 345-349, wherein the suicide gene or suicide switch is selected from the group consisting of cytosine deaminase (CyD), herpesvirus thymidine kinase (HSV-Tk), an inducible caspase 9 (iCaspase9), and rapamycin-activated caspase 9 (rapaCasp9).

351. The method of any of embodiments 345-349, wherein the suicide gene or the suicide switch is activated to induce controlled cell death after the administration of the one or more immunosuppressive agents to the subject.

352. The method of any of embodiments 345-349, wherein the suicide gene or the suicide switch is activated to induce controlled cell death prior to the administration of the one or more immunosuppressive agents to the subject.

353. The method of any of embodiments 345-352, wherein the suicide gene or the suicide switch is activated to induce controlled cell death after the administration of the modified SC-beta cell to the subject.

354. The method of any of embodiments 345-353, wherein the suicide gene or the suicide switch is activated to induce controlled cell death in the event of cytotoxicity or other negative consequences to the subject.

355. The method of any of embodiments 316-354, comprising administering an agent that allows for depletion of a modified SC-beta cell of the population of modified SC-beta cells.

36. The method of embodiment 355, wherein the agent that allows for depletion of the modified SC-beta cell is an antibody that recognizes a protein expressed on the surface of the modified SC-beta cell.

357. The method of embodiment 356, wherein the antibody is selected from the group consisting of an antibody that recognizes CCR4, CD16, CD19, CD20, CD30, EGFR, GD2, HER1, HER2, MUC1, PSMA, and RQR8.

358. The method of embodiment 355 or embodiment 356, wherein the antibody is selected from the group consisting of mogamulizumab, AFM13, MOR208, obinutuzumab, ublituximab, ocaratuzumab, rituximab, rituximab-Rllb, tomuzotuximab, RO5083945 (GA201), cetuximab, Hul4.18K322A, Hul4.18-IL2, Hu3F8, dinituximab, c.60C3-Rllc, and biosimilars thereof.

359. The method of any of embodiments 303-315 and 355-358, comprising administering an agent that recognizes the one or more tolerogenic factors on the surface of the modified SC-beta cell.

360. The method of embodiment 359, wherein the modified SC-beta cell is engineered to express the one or more tolerogenic factors.

361. The method of embodiment 359 or embodiment 360, wherein the one or more tolerogenic factors is CD47.

362. The method of any of embodiments 303-361, further comprising administering one or more additional therapeutic agents to the subject.

363. The method of any of embodiments 303-362, wherein the subject has been administered one or more additional therapeutic agents.

364. The method of any of embodiments 303-363, further comprising monitoring the therapeutic efficacy of the method.

365. The method of any of embodiments 303-364, further comprising monitoring the prophylactic efficacy of the method.

366. The method of any of embodiments 303-365, wherein the method is repeated until a desired suppression of one or more disease symptoms occurs.

367. The modified SC-beta cell of any of embodiments 152-264 and 267-291, wherein the modified SC-beta cell comprises an exogenous polynucleotide encoding a suicide gene or a suicide switch.

368. The modified SC-beta cell of embodiment 367, wherein the suicide gene or suicide switch is selected from the group consisting of cytosine deaminase (CyD), herpesvirus thymidine kinase (HSV-Tk), an inducible caspase 9 (iCaspase9), and rapamycin-activated caspase 9 (rapaCasp9).

369. The modified SC-beta cell of embodiment 367 or embodiment 368, wherein the suicide gene or suicide switch and genes associated with the suicide gene or the safety switch are expressed from a bicistronic cassette integrated into the genome of the modified SC-beta cell.

370. The modified SC-beta cell of embodiment 367 or embodiment 368, wherein the suicide gene or suicide switch and the one or more tolerogenic factors are expressed from a bicistronic cassette integrated into the genome of the modified SC-beta cell.

371. The modified SC-beta cell of embodiment 369 or embodiment 370, wherein the bicistronic cassette is integrated by non-targeted insertion into the genome of the modified SC-beta cell, optionally by introduction of the exogenous polynucleotide into the cell using a lentiviral vector.

372. The modified SC-beta cell of embodiment 369 or 370, wherein the bicistronic cassette is integrated by targeted insertion into a target genomic locus of the cell, optionally wherein the targeted insertion is by nuclease-mediated gene editing with homology-directed repair.

373. The modified SC-beta cell of any of embodiments 366-372, wherein the one or more tolerogenic factors is CD47.

374. The method of any of embodiments 1-39, 42, 43, 48-115, and 118-150, wherein the modified SC-beta cell comprises an exogenous polynucleotide encoding a suicide gene or suicide switch.

375. The method of embodiment 374, wherein the suicide gene is selected from the group consisting of cytosine deaminase (CyD), herpesvirus thymidine kinase (HSV-Tk), an inducible caspase 9 (iCaspase9), and rapamycin-activated caspase 9 (rapaCasp9).

376. The method of embodiment 374 or embodiment 375, wherein the suicide gene or suicide switch and genes associated with the suicide gene or the safety switch are expressed from a bicistronic cassette integrated into the genome of the modified SC-beta cell.

377. The method of embodiment 374 or embodiment 375, wherein the suicide gene or suicide switch and the one or more tolerogenic factors are expressed from a bicistronic cassette integrated into the genome of the modified SC-beta cell.

378. The method of embodiment 376 or embodiment 377, wherein the bicistronic cassette is integrated by non-targeted insertion into the genome of the modified SC-beta cell

379. The method of embodiment 376 or embodiment 377, wherein the bicistronic cassette is integrated by targeted insertion into a target genomic locus of the modified SC-beta cell.

380. The method of any of embodiments 374-379, wherein the one or more tolerogenic factors is CD47.

381. The composition of any of embodiments 151 and 293-302, wherein modified SC-beta cells of the population of modified SC-beta cells comprise an exogenous polynucleotide encoding a suicide gene or a suicide switch.

382. The composition of embodiment 381, wherein the suicide gene or suicide switch is selected from the group consisting of cytosine deaminase (CyD), herpesvirus thymidine kinase (HSV-Tk), an inducible caspase 9 (iCaspase9), and rapamycin-activated caspase 9 (rapaCasp9).

383. The composition of embodiment 381 or embodiment 382, wherein the suicide gene and genes associated with the suicide gene or the safety switch are expressed from a bicistronic cassette integrated into the genome of modified SC-beta cells of the population of modified SC-beta cells.

384. The composition of embodiment 381 or embodiment 382, wherein the suicide gene or suicide switch and the exogenous CD47 are expressed from a bicistronic cassette integrated into the genome of the modified SC-beta cell.

385. The composition of embodiment 383 or embodiment 384, wherein the bicistronic cassette is integrated by non-targeted insertion into the genome, optionally by introduction of the exogenous polynucleotide into modified SC-beta cells of the population of modified SC-beta cells using a lentiviral vector.

386. The composition of embodiment 383 or embodiment 384, wherein the bicistronic cassette is integrated by targeted insertion into a target genomic locus of modified SC-beta cells of the population of modified SC-beta cells, optionally wherein the targeted insertion is by nuclease-mediated gene editing with homology-directed repair.

Also among the provided embodiments are:

1. A method of generating a hypoimmune pluripotent stem cell (PSC) derived beta cell (HIP SC-beta cell), the method comprising:
(1) providing a modified PSC comprising modifications that: (a) reduce or eliminate expression of MHC class I and/or class II human leukocyte antigens in the modified PSC; and (b) increase expression of a tolerogenic factor in the modified PSC, relative to control or wild-type PSC, optionally wherein the control or wild-type PSC is an unmodified PSC that does not comprise the modifications; and
(2) culturing the modified PSC under conditions for differentiation into the HIP SC-beta) cell.

2. The method of embodiment 1, wherein the modified pluripotent stem cell is reduced or eliminated for expression of MHC class I and MHC class II human leukocyte antigens.

3. The method of embodiment 1, wherein the modified pluripotent stem cell is eliminated for expression of MHC class I and/or MHC class II human leukocyte antigens.

4. The method of embodiment 1, wherein the modified pluripotent stem cell is eliminated for expression of MHC class I and MHC class II human leukocyte antigens.

5. The method of any of embodiments 1-4, the modification that reduces or eliminates expression of MHC class I comprises reduced or eliminated expression of B2M.

6. The method of any of embodiments 1-5, wherein the modification comprises:
inactivation or disruption of both alleles of the B2M gene;
inactivation or disruption of both alleles of the B2M gene;
inactivation or disruption of all B2M coding alleles in the cell;
optionally, wherein the inactivation or disruption comprises an indel in the B2M gene and/or is a frameshift mutation or a deletion of a contiguous stretch of genomic DNA of the B2M gene.

7. The method of any of embodiments 1-6, wherein the B2M gene is knocked out.

8. The method of any of embodiments 1-7, the modification that reduces or eliminates expression of MHC class II comprises reduced or eliminated expression of CIITA.

9. The method of any of embodiments 1-8, wherein the modification comprises:
inactivation or disruption of both alleles of the CIITA gene;
inactivation or disruption of both alleles of the CIITA gene;
inactivation or disruption of all CIITA coding alleles in the cell;
optionally, wherein the inactivation or disruption comprises an indel in the CIITA gene and/or is a frameshift mutation or a deletion of a contiguous stretch of genomic DNA of the CIITA gene.

10. The method of any of embodiments 1-9, wherein the CIITA gene is knocked out.

11. The method of any of 1-10, wherein the modification to increase expression of the tolerogenic factor (e.g. CD47) is by introduction of an exogenous polynucleotide encoding the tolerogenic factor protein (e.g. CD47 protein) into the unmodified pluripotent stem cell.

12. The method of any of embodiments 1-11, wherein the exogenous polynucleotide encoding CD47 is integrated into the genome of the modified pluripotent stem cell by targeted insertion into a target genomic locus of the cell, optionally wherein the targeted insertion is by nuclease-mediated gene editing with homology-directed repair.

13. The method of embodiment 12, wherein the target genomic locus is a safe harbor locus, a B2M gene locus, a CIITA gene locus, or a CD142 gene locus.

14. The method of embodiment 12 or 13, wherein the target genomic locus is selected from the group consisting of: a CCR5 gene locus, a CXCR4 gene locus, a PPP1R12C (also known as AAVS1) gene, an albumin gene locus, a SHS231 locus, a CLYBL gene locus, and a ROSA26 gene locus.

15. The method of any of embodiment 1-14, wherein the modified pluripotent stem cell further comprises expression of an exogenous suicide gene selected from the group consisting of cytosine deaminase (CyD), herpesvirus thymidine kinase (HSV-Tk), an inducible caspase (e.g. iCaspase9), and rapamycin-activated caspase 9 (rapaCasp9).

16. The method of embodiment 15, wherein the suicide gene and CD47 are expressed from a bicistronic cassette introduced (e.g. integrated into the target genomic locus) of the modified pluripotent stem cell.

17. The method of any of embodiments 1-16, wherein the modified pluripotent stem cell comprises a modification that reduces or eliminates expression of CD142.

18. The method of embodiment 17, wherein the modified pluripotent stem cell comprises a modification that eliminates expression of CD142.

19. The method of embodiment 17 or embodiment 18, wherein the modification comprises:
inactivation or disruption of both alleles of the CD142 gene;
inactivation or disruption of both alleles of the CD142 gene;
inactivation or disruption of all CD142 coding alleles in the cell;
optionally, wherein the inactivation or disruption comprises an indel in the CD142 gene and/or is a frameshift mutation or a deletion of a contiguous stretch of genomic DNA of the CD142 gene.

20. The method of any of embodiments 17-19, wherein CD142 gene is knocked out.

21. The method of any of embodiments 1-20, wherein the modified pluripotent stem cell comprises a modification that increases expression of one or more complement inhibitors selected from the group consisting of CD46, CD59, and CD55 in said cell.

22. The method of embodiment 21, wherein the modification to increase expression of the one or more complement inhibitors is by introduction of at least one exogenous polynucleotide encoding CD46, an exogenous polynucleotide encoding CD59 and/or an exogenous polynucleotide encoding CD55 to the unmodified pluripotent stem cell.

23. A method of generating a hypoimmune beta cell derived from a pluripotent stem cell, the method comprising:
(1) generating a modified pluripotent stem cell comprising:
   (a) reducing or eliminating expression of MHC class I and/or class II human leukocyte antigens in a pluripotent stem cell; and
   (b) increasing expression of a tolerogenic factor in the cell; and
(2) culturing the modified pluripotent stem cell under conditions for differentiation into a modified stem-cell derived beta islet (modified SC-beta) cell.

24. The method of embodiment 23, wherein generating the modified pluripotent stem cell comprises reducing the expression of MHC class I and MHC class II human leukocyte antigens.

25. The method of embodiment 23, wherein generating the modified pluripotent stem cell comprises eliminating the expression of MHC class I and/or MHC class II human leukocyte antigens.

26. The method of embodiment 23 or embodiment 25, wherein generating the modified pluripotent stem cell comprises eliminating the expression of MHC class I and MHC class II human leukocyte antigens.

27. The method of any of embodiments 23-26, wherein reducing or eliminating expression of MHC class I human leukocyte antigens comprises introducing a modification that reduces or eliminates MHC class I protein expression.

28. The method of embodiment 27, wherein the modification that reduces or eliminates MHC class I protein expression comprises reduced or eliminated expression of B2M.

29. The method of embodiment 27 or embodiment 28, wherein:
the modification that reduces or eliminates MHC class I human leukocyte antigen expression comprises inactivation or disruption of both alleles of the B2M gene; or
the modification that reduces or eliminates MHC class I protein expression comprises inactivation or disruption of all B2M coding sequences in the cell,
optionally wherein inactivation or disruption comprises an indel in the B2M gene or a deletion of a contiguous stretch of genomic DNA of the B2M gene, optionally wherein the indel is a frameshift mutation.

30. The method of any of embodiment 23-29, wherein reducing or eliminating expression of MHC class II comprises introducing a modification that reduces or eliminates MHC class II protein expression.

31. The method of embodiment 30, wherein the modification that reduces or eliminates MHC class II protein expression comprises reducing or eliminating expression of CIITA.

32. The method of embodiment 30 or embodiment 31, wherein:
the modification that reduces or eliminates MHC class II protein expression comprises inactivation or disruption of both alleles of the CIITA gene; or
wherein the modification that reduces or eliminates MHC class II protein expression comprises inactivation or disruption of all CIITA coding sequences in the cell,
optionally wherein the inactivation or disruption comprises an indel in the CIITA gene or a deletion of a contiguous stretch of genomic DNA of the CIITA gene, optionally wherein the indel is a frameshift mutation.

33. The method of any of 23-32, wherein increasing expression of the tolerogenic factor (e.g. CD47) comprises introducing an exogenous polynucleotide encoding the tolerogenic factor protein (e.g. CD47 protein) into the unmodified pluripotent stem cell.

34. The method of any of embodiments 23-33, wherein the introduction integrates the exogenous polynucleotide encoding the tolerogenic factor (e.g. CD47) into the genome of the modified pluripotent stem cell.

35. The method of embodiment 34, wherein the integration is by targeted insertion into a target genomic locus of the cell, optionally wherein the targeted insertion is by nuclease-mediated gene editing with homology-directed repair.

36. The method of embodiment 35, wherein the target genomic locus is a safe harbor locus, a B2M gene locus, a CIITA gene locus, or a CD142 gene locus.

37. The method of embodiment 35 or 36, wherein the target genomic locus is selected from the group consisting of: a CCR5 gene locus, a CXCR4 gene locus, a PPP1R12C (also known as AAVS1) gene, an albumin gene locus, a SHS231 locus, a CLYBL gene locus, and a ROSA26 gene locus.

38. The method of any of embodiment 23-37, wherein generating the modified pluripotent stem cell further comprises introducing an exogenous suicide gene into the pluripotent stem cell, wherein the suicide gene is selected from the group consisting of cytosine deaminase (CyD), herpesvirus thymidine kinase (HSV-Tk), an inducible caspase (e.g. iCaspase9), and rapamycin-activated caspase 9 (rapaCasp9).

39. The method of embodiment 38, wherein the suicide gene and tolerogenic factor (e.g. CD47) are expressed from a bicistronic cassette introduced into the modified pluripotent stem cell, e.g. integrated into the target genomic locus of the modified pluripotent stem cell.

40. The method of any of embodiments 23-39, wherein generating the modified pluripotent stem cell further comprises reducing or eliminating the expression of CD142 in the cell.

41. The method of embodiment 40, wherein reducing or eliminating expression of CD142 comprises introducing a modification that reduces or eliminates CD142 protein expression.

42. The method of embodiment 41, wherein:
the modification that reduces or eliminates CD142 protein expression comprises inactivation or disruption of both alleles of the CD142 gene; or
wherein the modification that reduces or eliminates CD142 protein expression comprises inactivation or disruption of all CD142 coding sequences in the cell,
optionally wherein the inactivation or disruption comprises an indel in the CD142 gene or a deletion of a contiguous stretch of genomic DNA of the CD142 gene, optionally wherein the indel is a frameshift mutation.

43. The method of any of embodiments 23-42, wherein generating the modified pluripotent stem cell further comprises increasing the expression of one or more complement inhibitors selected from the group consisting of CD46, CD59, and CD55 in said cell.

44. The method of embodiment 43, wherein increasing expression of the one or more complement inhibitors comprises introducing at least one exogenous polynucleotide encoding CD46, an exogenous polynucleotide encoding CD59 and/or an exogenous polynucleotide encoding CD55 to the unmodified pluripotent stem cell.

45. The method of embodiment 21, embodiment 22, embodiment 43 or embodiment 44, wherein the one or more complement inhibitors is CD46 and CD59.

46. The method of embodiment 21, embodiment 22, embodiment 43 or embodiment 44, wherein the one or more complement inhibitor is CD46, CD59 and CD55.

47. The method of any of embodiments 1-46, wherein the culturing the modified pluripotent stem cell under conditions for differentiation into the modified SC-beta cell comprises one or more of:
(i) contacting the modified pluripotent stem cell with a TGF/Activin agonist or a glycogen synthase kinase 3 (GSK) inhibitor or WNT agonist for an amount of time sufficient to form a definitive endoderm cell;
(ii) contacting a definitive endoderm cell with a FGFR2b agonist for an amount of time sufficient to form a primitive gut tube cell;
(iii) contacting a primitive gut tube cell with an RAR agonist, and optionally a rho kinase inhibitor, a smoothened antagonist, a FGFR2b agonist, a protein kinase C activator, or a BMP type 1 receptor inhibitor for an amount of time sufficient to form an early pancreas progenitor cell;
(iv) incubating an early pancreas progenitor cell for at least about 3 days and optionally contacting the early pancreas progenitor cell with a rho kinase inhibitor, a TGF-/Activin agonist, a smoothened antagonist, an FGFR2b agonist, or a RAR agonist for an amount of time sufficient to form a pancreatic progenitor cell;
(v) contacting a pancreatic progenitor cell with an Alk5 inhibitor, a gamma secretase inhibitor, SANT 1, Erbb1 (EGFR) or Erbb4 agonist, or a RAR agonist for an amount of time sufficient to form an endoderm cell, wherein during at least a portion of the contacting in (v) depolymerizing the actin cytoskeleton at a time and for an amount of time sufficient to increase differentiation efficiency; and/or
(vi) incubating an endoderm cell for an amount of time in serum-free media sufficient to form a beta cell, wherein within about 24 hours of incubation resizing the cell clusters.

48. The method of any of embodiments 1-46, wherein the culturing the modified pluripotent stem cell under conditions for differentiation into the modified SC-beta cell comprises:
(i) contacting the modified pluripotent stem cell with a TGF/Activin agonist or a glycogen synthase kinase 3 (GSK) inhibitor or WNT agonist for an amount of time sufficient to form a definitive endoderm cell;
(ii) contacting a definitive endoderm cell with a FGFR2b agonist for an amount of time sufficient to form a primitive gut tube cell;
(iii) contacting a primitive gut tube cell with an RAR agonist, and optionally a rho kinase inhibitor, a smoothened antagonist, a FGFR2b agonist, a protein kinase C activator, or a BMP type 1 receptor inhibitor for an amount of time sufficient to form an early pancreas progenitor cell;
(iv) incubating an early pancreas progenitor cell for at least about 3 days and optionally contacting the early pancreas progenitor cell with a rho kinase inhibitor, a TGF-/Activin agonist, a smoothened antagonist, an FGFR2b agonist, or a RAR agonist for an amount of time sufficient to form a pancreatic progenitor cell;
(v) contacting a pancreatic progenitor cell with an Alk5 inhibitor, a gamma secretase inhibitor, SANT 1, Erbb1 (EGFR) or Erbb4 agonist, or a RAR agonist for an amount of time sufficient to form an endoderm cell, wherein during at least a portion of the contacting in (v) depolymerizing the actin cytoskeleton at a time and for an amount of time sufficient to increase differentiation efficiency; and
(vi) incubating an endoderm cell for an amount of time in serum-free media sufficient to form a beta cell, wherein within about 24 hours of incubation resizing the cell clusters.

49. The method of embodiment 47 or embodiment 48, wherein depolymerizing the actin cytoskeleton comprises plating cells on a stiff or soft substrate or introducing a cytoskeletal-modulating agent to cells.

50. The method of embodiment 49, wherein the cytoskeletal-modulating agent comprises latrunculin A, latrunculin B, nocodazole, cytochalasin D, jasplakinolide, blebbistatin, y-27632, y-15, gdc-0994, or an integrin modulating agent.

51. The method of embodiment 49 or embodiment 50, wherein the cytoskeletal-modulating agent is latrunculin A.

52. The method of any of embodiments 47-51, wherein depolymerizing the actin cytoskeleton is initiated at the start of the contacting in (v).

53. The method of any of embodiments 47-52, wherein depolymerizing the actin cytoskeleton comprises adding latrunculin A at the start of the contacting for at least at or about the first 24 hours.

54. The method of any of embodiments 47-53, wherein resizing the cell clusters comprises breaking apart clusters and reaggregating.

55. The method of any of embodiments 47-54, wherein:
the TGF/Activin agonist is Activin A;
the glycogen synthase kinase 3 (GSK) inhibitor or the WNT agonist is CHIR;
the FGFR2b agonist is KGF;
the smoothened antagonist is SANT-1;
the RAR agonist is retinoic acid (RA);
the protein kinase C activator is TPPB;
the BMP type 1 receptor inhibitor is LDN;
the rho kinase inhibitor is Y27632;
the Alk5 inhibitor is Alk5i;
the Erbb4 agonist is betacellulin;
the thyroid hormone is T3; and/or
the gamma secretase inhibitor is XXI.

56. The method of any of embodiments 1-55, wherein the pluripotent stems cells are embryonic stem cells.

57. The method of any of embodiments 1-55, wherein the pluripotent stem cells are induced pluripotent stem cells, optionally a patient-derived iPSC.

58. The method of any of embodiments 1-57, wherein the one or more tolerogenic factor is selected from the group consisting of CD47, CD27, CD200, HLA-C, HLA-E, HLA-E heavy chain, HLA-G, PD-L1, IDO1, CTLA4-Ig, Cl-Inhibitor, IL-10, IL-35, FASL, CCL21, MFGE8, and SERPINB9, and any combination thereof.

59. The method of any of embodiments 1-58, wherein at least one of the one or more tolerogenic factor is CD47.

60. The method of any of embodiments 1-58, wherein at least one of the one or more tolerogenic factor is PD-L1.

61. The method of any of embodiments 1-58, wherein at least one of the one or more tolerogenic factor is HLA-E.

62. The method of any of embodiments 1-58, wherein at least one of the one or more tolerogenic factor is HLA-G.

63. The method of any of embodiments 1-62, wherein the modified pluripotent stem cell expresses the tolerogenic factor (e.g. CD47) at a level that is greater than at or about 5-fold over the unmodified pluripotent stem cell.

64. The method of embodiment 63, wherein the tolerogenic factor (e.g. CD47) is expressed at a level that is greater than at or about 10-fold, greater than at or about 20-fold, greater than at or about 30-fold, greater than at or about 40-fold, greater than at or about 50-fold, greater than at or about 60-fold, or greater than at or about 70-fold over the unmodified pluripotent stem cell.

65. The method of any of embodiments 1-64, wherein the tolerogenic factor (e.g. CD47) is expressed by the modified pluripotent stem cell at greater than at or about 20,000 molecules per cell.

66. The method of embodiment 65, wherein the tolerogenic factor (e.g. CD47) is expressed by the modified pluripotent stem cell at greater than at or about 30,000 molecules per cell, greater than at or about 50,000 molecules per cell, greater than at or about 100,000 molecules per cell, greater than at or about 200,000 molecules per cell, greater than at or about 300,000 molecules per cell, greater than at or about 400,000 molecules per cell, greater than at or about 500,000 molecules per cell, or greater than at or about 600,000 molecules per cell.

67. The method of any of embodiments 1-66, wherein the modified SC-beta cell is (a) reduced or eliminated for expression of MHC class I and/or class II human leukocyte antigens; and (b) increased for the expression of an exogenous tolerogenic factor (e.g. CD47), compared to an unmodified SC-beta cell differentiated from an unmodified pluripotent stem cell not comprising the modifications.

68. The method of any of embodiments 1-67, wherein the modified SC-beta cell is reduced or eliminated for expression of MHC class I and MHC class II human leukocyte antigens.

69. The method of any of embodiments 1-67, wherein the modified SC-beta cell is eliminated for expression of MHC class I and/or MHC class II human leukocyte antigens.

70. The method of any of embodiments 1-67 and 69, wherein the modified SC-beta cell is eliminated for expression of MHC class I and MHC class II human leukocyte antigens.

71. The method of any of embodiments 1-70, wherein the modified SC-beta cell comprises:
inactivation or disruption of both alleles of the B2M gene;
inactivation or disruption of both alleles of the B2M gene;
inactivation or disruption of all B2M coding alleles in the cell;
optionally, wherein the inactivation or disruption comprises an indel in the B2M gene and/or is a frameshift mutation or a deletion of a contiguous stretch of genomic DNA of the B2M gene.

72. The method of any of embodiments 1-71, wherein the B2M gene is knocked out in the modified SC-beta cell.

73. The method of any of embodiments 1-72, wherein the modified SC-beta cell comprises:
inactivation or disruption of both alleles of the CIITA gene;
inactivation or disruption of both alleles of the CIITA gene;
inactivation or disruption of all CIITA coding alleles in the cell;
optionally, wherein the inactivation or disruption comprises an indel in the CIITA gene and/or is a frameshift mutation or a deletion of a contiguous stretch of genomic DNA of the CIITA gene.

74. The method of any of embodiments 1-73, wherein the CIITA gene is knocked out in the modified SC-beta cell.

75. The method of any of embodiments 1-74, wherein:
the modified SC-beta cell is reduced or eliminated for expression of CD142; and/or
wherein the modified SC-beta cell comprises:
inactivation or disruption of both alleles of the CD142 gene;
inactivation or disruption of both alleles of the CD142 gene;
inactivation or disruption of all CD142 coding alleles in the cell;
optionally, wherein the inactivation or disruption comprises an indel in the CD142 gene and/or is a frameshift mutation or a deletion of a contiguous stretch of genomic DNA of the CD142 gene.

76. The method of any of embodiments 1-75, wherein the CD142 gene is knocked out in the modified SC-beta cell.

77. The method of any of embodiments 1-76, wherein the modified SC-beta cell comprises an exogenous polynucleotide encoding the tolerogenic factor (e.g. CD47).

78. The method of any of embodiments 1-77, wherein the exogenous polynucleotide encoding the tolerogenic factor (e.g. CD47) is integrated into the genome of the modified SC-beta cell at a target genomic locus of the cell.

79. The method of embodiment 78, wherein the target genomic locus is a safe harbor locus, a B2M gene locus, a CIITA gene locus, or a CD142 gene locus.

80. The method of embodiment 78 or embodiment 79, wherein the target genomic locus is selected from the group consisting of: a CCR5 gene locus, a CXCR4 gene locus, a PPP1R12C (also known as AAVS1) gene, an albumin gene locus, a SHS231 locus, a CLYBL gene locus, and a ROSA26 gene locus.

81. The method of any of embodiment 1-80, wherein the modified SC-beta cell further comprises expression of an exogenous suicide gene selected from the group consisting of cytosine deaminase (CyD), herpesvirus thymidine kinase (HSV-Tk), an inducible caspase (e.g. iCaspase9), and rapamycin-activated caspase 9 (rapaCasp9).

82. The method of embodiment 81, wherein the suicide gene and the tolerogenic factor (e.g. CD47) are expressed from a bicistronic cassette integrated into the target genomic locus of the modified SC-beta cell.

83. The method of any of embodiments 1-82, wherein the modified SC-beta cell comprises increased expression of one or more complement inhibitors selected from the group consisting of CD46, CD59, and CD55, compared to an unmodified SC-beta cell differentiated from an unmodified pluripotent stem cell not comprising the modifications.

84. The method of embodiment 83, wherein the modified cell comprises at least one exogenous polynucleotide encoding the one or more complement inhibitors.

85. The method of any of embodiments 1-84, wherein the reduced or eliminated expression comprises reduced or eliminated surface expression.

86. The method of any of embodiments 1-85, wherein the increased expression comprises increased surface expression.

87. The method of any of embodiments 67-86, wherein the level of the reduced or eliminated expression of (a) and the increased expression of (b) by the modified SC-beta cell is retained or is similar compared to the modified pluripotent stem cell.

88. The method of any of embodiments 67-87, wherein the modified SC-beta cell expresses the tolerogenic factor (e.g. CD47) at a level that is greater than at or about 5-fold over an unmodified SC-beta cell differentiated from an unmodified pluripotent stem cell.

89. The method of any of embodiments 67-88, wherein the modified SC-beta cell expresses the tolerogenic factor (e.g. CD47) at a level that is greater than at or about 5-fold over the unmodified SC-beta cell differentiated from an unmodified pluripotent stem cell.

90. The method of embodiment 89, wherein the tolerogenic factor (e.g. CD47) is expressed at a level that is greater than at or about 10-fold, greater than at or about 20-fold, greater than at or about 30-fold, greater than at or about 40-fold, greater than at or about 50-fold, greater than at or about 60-fold, or greater than at or about 70-fold over the unmodified SC-beta cell.

91. The method of any of embodiments 67-90, wherein the tolerogenic factor (e.g. CD47) is expressed by the modified SC-beta cell at greater than at or about 20,000 molecules per cell.

92. The method of embodiment 91, wherein the tolerogenic factor (e.g. CD47) is expressed by the modified SC-beta cell at greater than at or about 30,000 molecules per cell, greater than at or about 50,000 molecules per cell, greater than at or about 100,000 molecules per cell, greater than at or about 200,000 molecules per cell, greater than at or about 300,000 molecules per cell, greater than at or about 400,000 molecules per cell, greater than at or about 500,000 molecules per cell, or greater than at or about 600,000 molecules per cell.

93. The method of any of embodiments 1-92, wherein the modified SC-beta cell expresses at least one beta cell marker, optionally wherein the beta cell marker is selected from the group consisting of INS, CHGA, NKX2-2, PDX1, NKX6-1, MAFB, GCK and GLUT1.

94. The method of any of embodiments 1-93, wherein the modified SC-beta cell is capable of glucose-stimulated insulin secretion (GSIS), optionally wherein the insulin secretion is in a perfusion GSIS assay.

95. The method of embodiment 94, wherein the GSIS is dynamic GSIS comprising first and second phase dynamic insulin secretion.

96. The method of embodiment 95, wherein the GSIS is static GSIS, optionally wherein the static incubation index is greater than at or about 1, greater than at or about 2, greater than at or about 5, greater than at or about 10 or greater than at or about 20.

97. The method of any of embodiments 94-96, wherein the level of insulin secretion by the modified SC-beta cells is at least 20% of that observed for primary beta islets, optionally cadaveric islets. 98. The method of embodiment 97, wherein the level of insulin secretion by the modified SC-beta cells is at least 25%, at least 30%, at least 40%, at least 50%, at least 60%, at least 70% of that observed for primary beta islets, optionally cadaveric islets.

99. The method of any of embodiments 1-98, wherein the total insulin content of the modified SC-beta cell is greater than at or about 500 μIU Insulin per 5000 cells, greater than at or about 1000 μIU Insulin per 5000 cells, greater than at or about 2000 μIU Insulin per 5000 cells, greater than at or about 3000 μIU Insulin per 5000 cells or greater than at or about 4000 μIU Insulin per 5000 cells.

100. The method of any of embodiments 1-99, wherein the proinsulin to insulin ratio of the modified SC-beta cell is between at or about 0.02 and at or about 0.1, optionally at or about 0.03, 0.04, 0.05, 0.06, 0.07, 0.08, 0.09 and any value between any of the foregoing.

101. The method of any of embodiments 1-100, wherein the modified SC-beta cell retains functionality for 1 or more days.

102. The method of any of embodiments 1-101, wherein the modified SC-beta cells retain functionality for more than 1 week.

103. A composition comprising a population of modified SC-beta cells produced by the method of any of embodiments 1-102.

104. A modified stem-cell derived beta (SC-beta) cell that has been differentiated in vitro from a pluripotent stem cell, wherein the modified SC-beta has (a) reduced or eliminated expression of MHC class I and/or MHC class II human leukocyte antigens; and (b) increased expression of a tolerogenic factor (e.g. CD47), compared to a wild-type primary beta cell, and wherein the modified beta cell exhibits glucose-stimulated insulin secretion (GSIS).

105. The modified SC-beta cell of embodiment 104, wherein the tolerogenic factor (e.g. CD47) is expressed at a level that is greater than at or about 10-fold, greater than at or about 20-fold, greater than at or about 30-fold, greater than at or about 40-fold, greater than at or about 50-fold, greater than at or about 60-fold, or greater than at or about 70-fold over the wild-type primary beta cell.

106. A modified stem cell-derived beta (SC-beta) cell that has been differentiated in vitro from a pluripotent stem cell, wherein the modified SC-beta (a) does not express MHC class I or MHC class II human leukocyte antigens and (b) overexpresses a tolerogenic factor (e.g. CD47) at a level of greater than at or about 5-fold compared to background (e.g. isotype control), and wherein the modified SC-beta cell exhibits glucose-stimulated insulin secretion (GSIS).

107. The modified SC-beta cell of embodiment 106, wherein the tolerogenic factor (e.g. CD47) is expressed at a level that is greater than at or about 10-fold, greater than at or about 20-fold, greater than at or about 30-fold, greater than at or about 40-fold, greater than at or about 50-fold, greater than at or about 60-fold, or greater than at or about 70-fold compared to background (e.g. isotype control).

108. A modified stem cell-derived beta (SC-beta) cell that has been differentiated in vitro from a pluripotent stem cell, wherein the modified SC-beta (a) does not express MHC class I or MHC class II human leukocyte antigens and (b) overexpresses a tolerogenic factor (e.g. CD47) at a level of greater than at or about 5-fold compared to an unmodified cell, wherein:
the unmodified cell is an unmodified pluripotent stem cell that does not comprise modifications to reduce or eliminate MHC class I or MHC class II human leukocyte antigens or to overexpress the tolerogenic factor (e.g. CD47) or is an unmodified SC-beta cell differentiated from such unmodified pluripotent stem cell;
the modified SC-beta cell exhibits glucose-stimulated insulin secretion (GSIS).

109. The modified SC-beta cell of any of embodiments 104-108, wherein the tolerogenic factor (e.g. CD47) is expressed by the modified SC-beta cell at greater than at or about 20,000 molecules per cell.

110. A modified stem cell-derived beta (SC-beta) cell that has been differentiated in vitro from a pluripotent stem cell, wherein the modified SC-beta cell (a) does not express MHC class I or MHC class II human leukocyte antigens and (b) expresses a tolerogenic factor (e.g. CD47) at greater than at or about 20,000 molecules per cell, and wherein the modified beta cell exhibits glucose-stimulated insulin secretion (GSIS).

111. The modified SC-beta cell of embodiment 100 or embodiment 110, wherein the tolerogenic factor (e.g. CD47) is expressed by the modified SC-beta at greater than at or about 30,000 molecules per cell, greater than at or about 50,000 molecules per cell, greater than at or about 100,000 molecules per cell, greater than at or about 200,000 molecules per cell, greater than at or about 300,000 molecules per cell, greater than at or about 400,000 molecules per cell, greater than at or about 500,000 molecules per cell, or greater than at or about 600,000 molecules per cell.

112. The modified SC-beta cell of any of embodiments 104-111, wherein the pluripotent stem cell is a modified pluripotent stem cell comprising modifications that (a) reduce or eliminate expression of MHC class I or MHC class II human leukocyte antigens; and (b) increase expression of a tolerogenic factor (e.g. CD47), relative to an unmodified pluripotent stem cell that does not comprise the modifications.

113. The modified SC-beta cell of embodiment 108 or embodiment 112, wherein the modified SC-beta expresses the tolerogenic factor (e.g. CD47) at a level that is greater than at or about 5-fold over the unmodified pluripotent stem cell or the unmodified SC-beta differentiated from the unmodified pluripotent stem cell.

114. The modified SC-beta cell of embodiment 113, wherein the tolerogenic factor (e.g. CD47) is expressed at a level that is greater than at or about 10-fold, greater than at or about 20-fold, greater than at or about 30-fold, greater than at or about 40-fold, greater than at or about 50-fold, greater than at or about 60-fold, or greater than at or about 70-fold compared to the unmodified pluripotent stem cell or an unmodified SC-beta differentiated from the unmodified pluripotent stem cell.

115. A modified stem-cell derived beta (SC-beta) cell that has been differentiated in vitro from a modified pluripotent stem cell, wherein the modified pluripotent stem cell comprises modification that (a) has reduce or eliminate expression of MHC class I or MHC class II human leukocyte antigens; and (b) increase expression of a tolerogenic factor (e.g. CD47), relative to an unmodified pluripotent stem cell that does not comprise the modifications, and wherein the modified SC-beta cell exhibits glucose-stimulated insulin secretion (GSIS).

116. The modified SC-beta cell of any of embodiments 110-115, wherein the modified pluripotent stem cell expresses the tolerogenic factor (e.g. CD47) at a level that is greater than at or about 5-fold over an unmodified pluripotent stem cell that does not comprise the modifications, optionally wherein the tolerogenic factor (e.g. CD47) is expressed at a level that is greater than at or about 10-fold, greater than at or about 20-fold, greater than at or about 30-fold, greater than at or about 40-fold, greater than at or about 50-fold, greater than at or about 60-fold, or greater than at or about 70-fold over the unmodified pluripotent stem cell.

117. The modified SC-beta cell of any of embodiments 112-116, wherein the modified SC-beta cell has reduced or eliminated expression of MHC class I or MHC class II human leukocyte antigens; and (b) increased expression of a tolerogenic factor (e.g. CD47), compared to the unmodified pluripotent stem cell or an unmodified SC-beta differentiated from the unmodified pluripotent stem cell. 118. The modified SC-beta cell of embodiment 117, wherein the modified SC-beta expresses the tolerogenic factor (e.g. CD47) at a level that is greater than at or about 5-fold over the unmodified pluripotent stem cell or the unmodified SC-beta cell differentiated from an unmodified pluripotent stem cell.

119. The modified SC-beta cell of embodiment 118, wherein the tolerogenic factor (e.g. CD47) is expressed at a level that is greater than at or about 10-fold, greater than at or about 20-fold, greater than at or about 30-fold, greater than at or about 40-fold, greater than at or about 50-fold, greater than at or about 60-fold, or greater than at or about 70-fold over the unmodified pluripotent stem cell or the unmodified SC-beta cell differentiated from an unmodified pluripotent stem cell.

120. The modified SC-beta of any of embodiments 112-119, wherein the tolerogenic factor (e.g. CD47) is expressed by the modified pluripotent stem cell at greater than at or about 20,000 molecules per cell.

121. A modified stem cell-derived beta (SC-beta) cell that has been differentiated in vitro from a modified pluripotent stem cell, wherein the modified pluripotent stem cell comprises modifications such that the modified pluripotent stem cell (a) does not express MHC class I or MHC class II human leukocyte antigens; and (b) expresses a tolerogenic factor (e.g. CD47) at greater than at or about 20,000 molecules per cell, and wherein the modified SC-beta cell exhibits glucose-stimulated insulin secretion (GSIS).

122. The modified SC-beta of embodiment 120 or 121, wherein the tolerogenic factor (e.g. CD47) is expressed by the modified pluripotent stem cell at greater than at or about 30,000 molecules per cell, greater than at or about 50,000 molecules per cell, greater than at or about 100,000 molecules per cell, greater than at or about 200,000 molecules per cell, greater than at or about 300,000 molecules per cell, greater than at or about 400,000 molecules per cell, greater than at or about 500,000 molecules per cell, or greater than at or about 600,000 molecules per cell.

123. The modified SC-beta cell of any of embodiments 112-122, wherein the modified SC-beta cell does not express MHC class I or MHC class II human leukocyte antigens and expresses the tolerogenic factor (e.g. CD47) at greater than at or about 20,000 molecules per cell.

124. The modified SC-beta cell of embodiment 123, wherein the tolerogenic factor (e.g. CD47) is expressed by the modified SC-beta at greater than at or about 30,000 molecules per cell, greater than at or about 50,000 molecules per cell, greater than at or about 100,000 molecules per cell, greater than at or about 200,000 molecules per cell, greater than at or about 300,000 molecules per cell, greater than at or about 400,000 molecules per cell, greater than at or about 500,000 molecules per cell, or greater than at or about 600,000 molecules per cell.

125. The modified SC-beta cell of any of embodiments 104-124, wherein the one or more tolerogenic factor is selected from the group consisting of CD47, CD27, CD200, HLA-C, HLA-E, HLA-E heavy chain, HLA-G, PD-L1, IDO1, CTLA4-Ig, Cl-Inhibitor, IL-10, IL-35, FASL, CCL21, MFGE8, and SERPINB9, and any combination thereof.

126. The modified SC-beta cell of any of embodiments 104-125, wherein at least one of the one or more tolerogenic factor is CD47.

127. The modified SC-beta cell of any of embodiments 104-125, wherein at least one of the one or more tolerogenic factor is PD-L1.

128. The modified SC-beta cell of any of embodiments 104-125, wherein at least one of the one or more tolerogenic factor is HLA-E.

129. The modified SC-beta cell of any of embodiments 104-125, wherein at least one of the one or more tolerogenic factor is HLA-G.

130. The modified SC-beta cell of any of embodiments 112-129, wherein the modified pluripotent stem cell is reduced or eliminated for expression of MHC class I and MHC class II human leukocyte antigens.

131. The modified SC-beta cell of any of embodiments 112-129, wherein the modified pluripotent stem cell is eliminated for expression of MHC class I and/or MHC class II human leukocyte antigens.

132. The modified SC-beta cell of any of embodiments 112-129 and 131, wherein the modified pluripotent stem cell is eliminated for expression of MHC class I and MHC class II human leukocyte antigens.

133. The modified SC-beta cell of any of embodiments 112-132, wherein the modified pluripotent stem cell comprises:
inactivation or disruption of both alleles of the B2M gene;
inactivation or disruption of both alleles of the B2M gene;
inactivation or disruption of all B2M coding alleles in the cell;
optionally, wherein the inactivation or disruption comprises an indel in the B2M gene and/or is a frameshift mutation or a deletion of a contiguous stretch of genomic DNA of the B2M gene.

134. The modified SC-beta cell of any of embodiments 112-133, wherein the B2M gene is knocked out in the modified pluripotent stem cell.

135. The modified SC-beta cell of any of embodiments 112-134, wherein the modified pluripotent stem cell comprises:
inactivation or disruption of both alleles of the CIITA gene;
inactivation or disruption of both alleles of the CIITA gene;
inactivation or disruption of all CIITA coding alleles in the cell;
optionally, wherein the inactivation or disruption comprises an indel in the CIITA gene and/or is a frameshift mutation or a deletion of a contiguous stretch of genomic DNA of the CIITA gene.

136. The modified SC-beta cell of any of embodiments 112-135, wherein the CIITA gene is knocked out in the modified pluripotent stem cell.

137. The modified SC-beta cell of any of embodiments 112-136, wherein the modified pluripotent stem cell comprises:
inactivation or disruption of both alleles of the CD142 gene;
inactivation or disruption of both alleles of the CD142 gene;
inactivation or disruption of all CD142 coding alleles in the cell;
optionally, wherein the inactivation or disruption comprises an indel in the CD142 gene and/or is a frameshift mutation or a deletion of a contiguous stretch of genomic DNA of the CD142 gene.

138. The modified SC-beta cell of any of embodiments 112-137, wherein the CD142 gene is knocked out in the modified pluripotent stem cell.

139. The modified SC-beta cell of any of embodiments 112-138, wherein the modified pluripotent stem cell comprises an exogenous polynucleotide encoding the tolerogenic factor (e.g. CD47).

140. The modified SC-beta cell of any of embodiments 112-139, wherein the exogenous polynucleotide encoding the tolerogenic factor (e.g. CD47) is integrated into the genome of the modified pluripotent stem cell at a target genomic locus of the cell.

141. The modified SC-beta cell of embodiment 140, wherein the target genomic locus is a safe harbor locus, a B2M gene locus, a CIITA gene locus, or a CD142 gene locus.

142. The modified SC-beta cell of embodiment 140 or embodiment 141, wherein the target genomic locus is selected from the group consisting of: a CCR5 gene locus, a CXCR4 gene locus, a PPP1R12C (also known as AAVS1) gene, an albumin gene locus, a SHS231 locus, a CLYBL gene locus, and a ROSA26 gene locus.

143. The modified SC-beta cell of any of embodiment 112-142, wherein the modified pluripotent stem cell further comprises expression of an exogenous suicide gene selected from the group consisting of cytosine deaminase (CyD), herpesvirus thymidine kinase (HSV-Tk), an inducible caspase (e.g. iCaspase9), and rapamycin-activated caspase 9 (rapaCasp9).

144. The modified SC-beta cell of embodiment 143, wherein the suicide gene and the tolerogenic factor (e.g. CD47) are expressed from a bicistronic cassette introduced in the modified pluripotent stem cell, e.g. integrated into the target geneomic locus of the modified pluripotent stem cell.

145. The modified SC-beta cell of any of embodiments 111-144, wherein the modified pluripotent stem cell comprises increased expression of one or more complement inhibitors selected from the group consisting of CD46, CD59, and CD55, compared to an unmodified SC-beta cell differentiated from an unmodified pluripotent stem cell not comprising the modifications.

146. The modified SC-beta cell of any of embodiments 112-145, wherein the modified pluripotent stem cell comprises at least one exogenous polynucleotide encoding one or more complement inhibitors.

147. The modified SC-beta cell of embodiment 145 or embodiment 146, wherein the one or more complement inhibitors is CD46 and CD59.

148. The modified SC-beta cell of embodiment 145 or embodiment 146, wherein the one or more complement inhibitor is CD46, CD59 and CD55.

149. The modified SC-beta cell of any of embodiments 104-148, wherein the modified SC-beta cell is reduced or eliminated for expression of MHC class I and MHC class II human leukocyte antigens.

150. The modified SC-beta cell of any of embodiments 104-148, wherein the modified SC-beta cell is eliminated for expression of MHC class I and/or MHC class II human leukocyte antigens.

151. The modified SC-beta cell of any of embodiments 104-148 and 150, wherein the modified SC-beta cell is eliminated for expression of MHC class I and MHC class II human leukocyte antigens.

152. The modified SC-beta cell of any of embodiments 104-151, wherein the modified SC-beta cell comprises:
inactivation or disruption of both alleles of the B2M gene;
inactivation or disruption of both alleles of the B2M gene;
inactivation or disruption of all B2M coding alleles in the cell;
optionally, wherein the inactivation or disruption comprises an indel in the B2M gene and/or is a frameshift mutation or a deletion of a contiguous stretch of genomic DNA of the B2M gene.

153. The modified SC-beta cell of any of embodiments 104-152, wherein the B2M gene is knocked out in the modified SC-beta cell.

154. The modified SC-beta cell of any of embodiments 104-153, wherein the modified SC-beta cell comprises:
inactivation or disruption of both alleles of the CIITA gene;
inactivation or disruption of both alleles of the CIITA gene;
inactivation or disruption of all CIITA coding alleles in the cell;
optionally, wherein the inactivation or disruption comprises an indel in the CIITA gene and/or is a frameshift mutation or a deletion of a contiguous stretch of genomic DNA of the CIITA gene.

155. The modified SC-beta cell of any of embodiments 104-154, wherein the CIITA gene is knocked out in the modified SC-beta cell.

156. The modified SC-beta cell of any of embodiments 104-155, wherein the modified SC-beta cell comprises:
inactivation or disruption of both alleles of the CD142 gene;
inactivation or disruption of both alleles of the CD142 gene;
inactivation or disruption of all CD142 coding alleles in the cell;
optionally, wherein the inactivation or disruption comprises an indel in the CD142 gene and/or is a frameshift mutation or a deletion of a contiguous stretch of genomic DNA of the CD142 gene.

157. The modified SC-beta cell of any of embodiments 104-156, wherein the CD142 gene is knocked out in the modified SC-beta cell.

158. The modified SC-beta cell of any of embodiments 104-157, wherein the modified SC-beta cell comprises an exogenous polynucleotide encoding the tolerogenic factor (e.g. CD47).

159. The modified SC-beta cell of any of embodiments 104-158, wherein the exogenous polynucleotide encoding the tolerogenic factor (e.g. CD47) is integrated into the genome of the modified SC-beta cell at a target genomic locus of the cell.

160. The modified SC-beta cell of embodiment 159, wherein the target genomic locus is a safe harbor locus, a B2M gene locus, a CIITA gene locus, or a CD142 gene locus.

161. The modified SC-beta cell of embodiment 159 or embodiment 160, wherein the target genomic locus is selected from the group consisting of: a CCR5 gene locus, a CXCR4 gene locus, a PPP1R12C (also known as AAVS1) gene, an albumin gene locus, a SHS231 locus, a CLYBL gene locus, and a ROSA26 gene locus.

162. The modified SC-beta cell of any of embodiment 104-161, wherein the modified SC-beta cell further comprises expression of an exogenous suicide gene selected from the group consisting of cytosine deaminase (CyD), herpesvirus thymidine kinase (HSV-Tk), an inducible caspase (e.g. iCaspase9), and rapamycin-activated caspase 9 (rapaCasp9).

163. The modified SC-beta cell of embodiment 162, wherein the suicide gene and the tolerogenic factor (e.g. CD47) are expressed from a bicistronic cassette integrated into the target genomic locus of the modified SC-beta cell.

164. The modified SC-beta cell of any of embodiments 104-163, wherein the modified SC-beta cell comprises increased expression of one or more complement inhibitors selected from the group consisting of CD46, CD59, and CD55, compared to an unmodified SC-beta cell differentiated from an unmodified pluripotent stem cell not comprising the modifications.

165. The modified SC-beta cell of embodiment 104-164, wherein the modified pluripotent stem cell comprises at least one exogenous polynucleotide encoding one or more complement inhibitors.

166. The modified SC-beta cell of embodiment 164 or embodiment 165, wherein the one or more complement inhibitors is CD46 and CD59.

167. The modified SC-beta cell of embodiment 164 or embodiment 165, wherein the one or more complement inhibitor is CD46, CD59 and CD55.

168. The modified SC-beta of any of embodiments 104-167, wherein the modified SC-beta cell expresses at least one beta cell marker, optionally wherein the beta cell marker is selected from the group consisting of INS, CHGA, NKX2-2, PDX1, NKX6-1, MAFB, GCK and GLUT1.

169. The modified SC-beta of any of embodiments 104-168, wherein the GSIS is measured in a perfusion GSIS assay.

170. The modified SC-beta of any of embodiments 104-169, wherein the GSIS is dynamic GSIS comprising first and second phase dynamic insulin secretion.

171. The modified SC-beta of any of embodiments 104-170, wherein the GSIS is static GSIS, optionally wherein the static stimulation index is greater than at or about 1, greater than at or about 1.5, greater than at or about 2, greater than at or about 5, greater than at or about 10, greater than at or about 15, or greater than at or about 20.

172. The modified SC-beta of any of embodiments 104-171, wherein the level of insulin secretion by the modified SC-beta cells is at least 20% of that observed for primary beta islets, optionally cadaveric islets.

173. The modified SC-beta of any of embodiments 104-172, wherein the level of insulin secretion by the modified SC-beta cells is at least 25%, at least 30%, at least 40%, at least 50%, at least 60%, at least 70% of that observed for primary beta islets, optionally cadaveric islets.

174. The modified SC-beta of any of embodiments 104-173, wherein the total insulin content of the modified SC-beta is greater than at or about 500 μIU Insulin per 5000 cells, greater than at or about 1000 μIU Insulin per 5000 cells, greater than at or about 2000 μIU Insulin per 5000 cells, greater than at or about 3000 μIU Insulin per 5000 cells or greater than at or about 4000 μIU Insulin per 5000 cells.

175. The modified SC-beta of any of embodiments 104-174, wherein the proinsulin to insulin ratio of the modified SC-beta is between at or about 0.02 and at or about 0.1, optionally at or about 0.03, 0.04, 0.05, 0.06, 0.07, 0.08, 0.09, and any value between any of the foregoing.

176. The modified SC-beta of any of embodiments 104-175, wherein the modified SC-beta cell retains functionality for 1 or more days.

177. The modified SC-beta of any of embodiments 104-176, wherein the modified SC-beta cell retain functionality for more than 1 week.

178. A composition comprising a modified SC-beta cell of any of embodiments 104-177.

179. A composition comprising a population of modified SC-beta cells of any of embodiments 104-177.

180. The composition of embodiment 103 or embodiment 179, wherein, among the cells in the population, the level of the reduced or eliminated expression of MHC class I and/or MHC class II human leukocyte antigens and/or the level of the increased expression of the tolerogenic factor (CD47) is retained or is similar compared to the modified pluripotent stem cell in at least at or about 50%, at least at or about 60%, at least at or about 70%, at least at or about 80%, or at least at or about 90% of the cells in the population.

181. The composition of embodiment 103, embodiment 179 or embodiment 180, wherein at least at or about 50%, at least at or about 60%, at least at or about 70%, at least at or about 80%, or at least at or about 90% of the cells in the population are eliminated for expression of MHC class I or for B2M.

182. The composition of any of embodiments 103 or 179-181, wherein at least at or about 50%, at least at or about 60%, at least at or about 70%, at least at or about 80%, or at least at or about 90% of the cells in the population are eliminated for expression of MHC class II or for CIITA.

183. The composition of any of embodiments 103 or 179-182, wherein at least at or about 50%, at least at or about 60%, at least at or about 70%, at least at or about 80%, or at least at or about 90% of the cells in the population level have increased expression of the tolerogenic factor (CD47) that is greater than at or about 5-fold, greater than at or about 10-fold, greater than at or about 20-fold, greater than at or about 30-fold, greater than at or about 40-fold, greater than at or about 50-fold, greater than at or about 60-fold, or greater than at or about 70-fold over a wild-type primary beta cell.

184. The composition of any of embodiments 103 or 179-183, wherein at least at or about 50%, at least at or about 60%, at least at or about 70%, at least at or about 80%, or at least at or about 90% of the cells in the population level have increased expression of the tolerogenic factor (CD47) that is greater than at or about 5-fold, greater than at or about 10-fold, greater than at or about 20-fold, greater than at or about 30-fold, greater than at or about 40-fold, greater than at or about 50-fold, greater than at or about 60-fold, or greater than at or about 70-fold over the unmodified pluripotent stem cell or an unmodified SC-beta differentiated from the unmodified pluripotent stem cell.

185. The composition of any of embodiments 103 or 179-184, wherein at least at or about 50%, at least at or about 60%, at least at or about 70%, at least at or about 80%, or at least at or about 90% of the cells in the population expresses the tolerogenic factor (e.g. CD47) at greater than at or about 20,000 molecules per cell, at greater than at or about 30,000 molecules per cell, greater than at or about 50,000 molecules per cell, greater than at or about 100,000 molecules per cell, greater than at or about 200,000 molecules per cell, greater than at or about 300,000 molecules per cell, greater than at or about 400,000 molecules per cell, greater than at or about 500,000 molecules per cell, or greater than at or about 600,000 molecules per cell.

186. The composition of any of embodiments 103 or 179-185, wherein at least at or about 50%, at least at or about 60%, at least at or about 70%, at least at or about 80%, or at least at or about 90% of the cells in the population are eliminated for expression of CD142.

187. The composition of any of embodiments 103 and 178-186 comprising a pharmaceutically acceptable excipient.

188. The composition of any of embodiments 103 and 178-187 comprising a cryoprotectant.

189. A method of treating diabetes (e.g., type I diabetes or type II diabetes) in a subject, the method comprising administering the modified SC-beta cells of any of embodiments 104-77 or the composition of any of embodiments 103 and 178-188 to a subject in need thereof.

190. The method of embodiment 189, wherein the beta cells improve glucose tolerance in the subject.

191. A method for improving glucose tolerance in a subject, the method comprising administering the modified SC-beta cells of any of embodiments 104-77 or the composition of any of embodiments 103 and 178-188 to a subject in need thereof.

192. The method of any of embodiments 189-191, wherein the subject is a diabetic patient.

193. The method of embodiment 192, wherein the diabetic patient has type I diabetes or type II diabetes.

194. The method of any of embodiments 190-193, wherein glucose tolerance is improved relative to the subject's glucose tolerance prior to administration of the islet cells.

195. The method of any of embodiments 189-194, wherein the modified SC-beta cells reduce exogenous insulin usage in the subject.

196. The method of any of embodiments 190-195, wherein glucose tolerance is improved as measured by HbA1c levels.

197. The method of any of embodiments 189-196, wherein the subject is fasting.

198. The method of any one of embodiments 189-197 wherein the modified SC-beta cells improve insulin secretion in the subject.

199. The method of embodiment 198, wherein insulin secretion is improved relative to the subject's insulin secretion prior to administration of the modified SC-beta cells.

Also among the provided embodiments are:

1. A modified stem-cell derived beta cell (SC-beta cell) comprising one or more modifications that: (a) inactivate or disrupt one or more alleles of: (i) one or more major histocompatibility complex (MHC) class I molecules or one or more molecules that regulate expression of the one or more MHC class I molecules, and/or (ii) one or more MHC class II molecules or one or more molecules that regulate expression of the one or more MHC class II molecules, and/or (b) increase expression of one or more tolerogenic factors, wherein the increased expression is relative to a control or wild-type beta cell that does not comprise the modifications.

2. The modified SC-beta cell of embodiment 1, wherein the one or more modifications in (i) reduce expression of:
a. one or more MHC class I molecules
b. one or more MHC class II molecules; or
c. one or more MHC class I molecules and one or more MHC class II molecules.

3. The modified SC-beta cell of embodiment 1 or embodiment 2, wherein the one or more modifications in (i) reduce expression of one or more molecules selected from the group consisting of B2M, TAP I, NLRC5, CIITA, HLA-A, HLA-B, HLA-C, HLA-DP, HLA-DQ, HLA-DR, HLA-DM, HLA-DO, RFX5, RFXANK, RFXAP, NFY-A, NFY-B, NFY-C, and any combination thereof.

4. The modified SC-beta cell of embodiment 3, wherein the modified SC-beta cell does not express one or more molecules selected from the group consisting of B2M, TAP I, NLRC5, CIITA, HLA-A, HLA-B, HLA-C, HLA-DP, HLA-DQ, HLA-DR, HLA-DM, HLA-DO, RFX5, RFXANK, RFXAP, NFY-A, NFY-B, NFY-C, and combinations thereof.

5. The modified SC-beta cell of any of embodiments 2-4, wherein the one or more modifications that increase expression comprise increased cell surface expression, and/or the one or more modifications that reduce expression comprise reduced cell surface expression.

6. The modified SC-beta cell of any of embodiments 1-5, wherein the one or more modifications in (i) reduce expression of one or more MHC class I molecules.

7. The modified SC-beta cell of any of embodiments 1-6, wherein the one or more modifications in (i) reduce expression of B2M.

8. The modified SC-beta cell of any of embodiments 1-7, wherein the one or more modifications in (i) reduce expression of HLA-A, HLA-B, and/or HLA-C.

9. The modified SC-beta cell of any of embodiments 1-8, wherein the one or more modifications in (i) reduce expression of one or more MHC class II molecules.

10. The modified SC-beta cell of any of embodiments 1-9, wherein the one or more modifications in (i) reduce expression of CIITA.

11. The modified SC-beta cell of any of embodiments 1-10, wherein the one or more modifications in (i) reduce expression of HLA-DM, HLA-DO, HLA-DP, HLA-DQ, HLA-DR, RFX5, RFXANK, and/or RFXAP.

12. The modified SC-beta cell of any of embodiments 1-11, wherein the one or more tolerogenic factors comprise one or more tolerogenic factors selected from the group consisting of A20/TNFAIP3, Cl-Inhibitor, CCL21, CCL22, CD16, CD16 Fc receptor, CD24, CD27, CD35, CD39, CD46, CD47, CD52, CD55, CD59, CD200, CR1, CTLA4-Ig, DUX4, FasL, H2-M3, HLA-C, HLA-E, HLA-E heavy chain, HLA-F, HLA-G, IDO1, IL-10, IL15-RF, IL-35, MANF, Mfge8, PD-L1, Serpinb9, and any combination thereof.

13. The modified SC-beta cell of any of embodiments 1-12, wherein the one or more tolerogenic factors comprise CD47.

14. The modified SC-beta cell of any of embodiments 1-13, wherein the one or more tolerogenic factors comprise CCL22.

15. The modified SC-beta cell of any of embodiments 1-14, wherein the one or more tolerogenic factors comprise CD16 or CD16 Fc receptor.

16. The modified SC-beta cell of any of embodiments 1-15, wherein the one or more tolerogenic factors comprise CD24.

17. The modified SC-beta cell of any of embodiments 1-16, wherein the one or more tolerogenic factors comprise CD39.

18. The modified SC-beta cell of any of embodiments 1-17, wherein the one or more tolerogenic factors comprise CR1.

19. The modified SC-beta cell of any of embodiments 1-18, wherein the one or more tolerogenic factors comprise CD52.

20. The modified SC-beta cell of any of embodiments 1-19, wherein the one or more tolerogenic factors comprise CD55.

21. The modified SC-beta cell of any of embodiments 1-20, wherein the one or more tolerogenic factors comprise CD200.

22. The modified SC-beta cell of any of embodiments 1-21, wherein the one or more tolerogenic factors comprise DUX4.

23. The modified SC-beta cell of any of embodiments 1-22, wherein the one or more tolerogenic factors comprise HLA-E.

24. The modified SC-beta cell of any of embodiments 1-23, wherein the one or more tolerogenic factors comprise HLA-G.

25. The modified SC-beta cell of any of embodiments 1-24, wherein the one or more tolerogenic factors comprise IDO1.

26. The modified SC-beta cell of any of embodiments 1-25, wherein the one or more tolerogenic factors comprise IL15-RF.

27. The modified SC-beta cell of any of embodiments 1-26, wherein the one or more tolerogenic factors comprise IL35.

28. The modified SC-beta cell of any of embodiments 1-27, wherein the one or more tolerogenic factors comprise PD-L1.

29. The modified SC-beta cell of any of embodiments 1-28, wherein the one or more tolerogenic factors comprise MANF.

30. The modified SC-beta cell of any of embodiments 1-29, wherein the one or more tolerogenic factors comprise A20/TNFAIP3.

31. The modified SC-beta cell of any of embodiments 1-30, wherein the one or more tolerogenic factors comprise HLA-E and CD47.

32. The modified SC-beta cell of any of embodiments 1-31, wherein the one or more tolerogenic factors comprise two or more tolerogenic factors selected from the group consisting of CD47, CD46, and CD59, optionally wherein the one or more tolerogenic factors comprise CD47, CD46, and CD59.

+

35. The modified SC-beta cell of any of embodiments 1-34, wherein the one or more tolerogenic factors comprise two or more tolerogenic factors selected from the group consisting of CD47, HLA-G and PD-L1, optionally wherein the one or more tolerogenic factors comprise CD47 and PD-L1, and optionally wherein the one or more tolerogenic factors comprise CD47, HLA-G and PD-L1.

36. The modified SC-beta cell of any of embodiments 1-35, wherein the one or more tolerogenic factors comprise two or more tolerogenic factors selected from the group consisting of CD24, CD47, and PD-L1, optionally wherein the one or more tolerogenic factors comprise CD24, CD47, and PD-L1.

37. The modified SC-beta cell of any of embodiments 1-36, wherein the one or more tolerogenic factors comprise two or more tolerogenic factors selected from the group consisting of HLA-E, CD24, CD47, and PD-L1, optionally wherein the one or more tolerogenic factors comprise HLA-E, CD24, CD47, and PD-L1.

38. The modified SC-beta cell of any of embodiments 1-37, wherein the one or more tolerogenic factors comprise two or more tolerogenic factors selected from the group consisting of CD46, CD55, CD59, and CR1, optionally wherein the one or more tolerogenic factors comprise CD46, CD55, CD59, and CR1.

39. The modified SC-beta cell of any of embodiments 1-38, wherein the one or more tolerogenic factors comprise two or more tolerogenic factors selected from the group consisting of HLA-E, CD46, CD55, CD59, and CR1, optionally wherein the one or more tolerogenic factors comprise HLA-E, CD46, CD55, CD59, and CR1.

40. The modified SC-beta cell of any of embodiments 1-39, wherein the one or more tolerogenic factors comprise two or more tolerogenic factors selected from the group consisting of HLA-E, CD24, CD47, PD-L1, CD46, CD55, CD59, and CR1, optionally wherein the one or more tolerogenic factors comprise HLA-E, CD24, CD47, PD-L1, CD46, CD55, CD59, and CR1.

41. The modified SC-beta cell of any of embodiments 1-40, wherein the one or more tolerogenic factors comprise HLA-E and PD-L1.

42. The modified SC-beta cell of any of embodiments 1-41, wherein the one or more tolerogenic factors comprise two or more tolerogenic factors selected from the group consisting of HLA-E, PD-L1, and A20/TNFAIP, optionally wherein the one or more tolerogenic factors comprise HLA-E, PD-L1, and A20/TNFAIP.

43. The modified SC-beta cell of any of embodiments 1-42, wherein the one or more tolerogenic factors comprise two or more tolerogenic factors selected from the group consisting of HLA-E, PD-L1, and MANF, optionally wherein the one or more tolerogenic factors comprise HLA-E, PD-L1, and MANF.

44. The modified SC-beta cell of any of embodiments 1-43, wherein the one or more tolerogenic factors comprise two or more tolerogenic factors selected from the group consisting of HLA-E, PD-L1, A20/TNFAIP, and MANF, optionally wherein the one or more tolerogenic factors comprise HLA-E, PD-L1, A20/TNFAIP, and MANF.

45. An modified SC-beta cell comprising one or more modifications that (i) reduce expression of one or more MHC class I molecules and one or more MHC class II molecules, and (ii) increase expression of CD47, wherein the reduced expression of (i) and the increased expression of (ii) is relative to a cell of the same cell type that does not comprise the modifications.

46. The modified SC-beta cell of embodiment 45, wherein the one or more modifications in (i) reduce expression of one or more molecules selected from the group consisting of B2M, TAP I, NLRC5, CIITA, HLA-A, HLA-B, HLA-C, HLA-DP, HLA-DQ, HLA-DR, HLA-DM, HLA-DO, RFX5, RFXANK, RFXAP, NFY-A, NFY-B, NFY-C, and any combination thereof.

47. The modified SC-beta cell of embodiment 45 or embodiment 46, wherein the one or more modifications in (i) reduce expression of B2M.

48. The modified SC-beta cell of any of embodiments 45-47, wherein the one or more modifications in (i) reduce expression of HLA-A, HLA-B, and/or HLA-C.

49. The modified SC-beta cell of any of embodiments 45-48, wherein the one or more modifications in (i) reduce expression of CIITA.

50. The modified SC-beta cell of any of embodiments 45-48, wherein the one or more modifications in (i) reduce expression of HLA-DP, HLA-DR, and/or HLA-DQ.

51. The modified SC-beta cell of any of embodiments 1-50, wherein the modified SC-beta cell further comprises one or more modifications that increase expression of one or more additional tolerogenic factors.

52. The modified SC-beta cell embodiment 51, wherein the one or more additional tolerogenic factors comprise one or more tolerogenic factors selected from the group consisting of A20/TNFAIP3, Cl-Inhibitor, CCL21, CCL22, CD16, CD16 Fc receptor, CD24, CD27, CD35, CD39, CD46, CD47, CD52, CD55, CD59, CD200, CR1, CTLA4-Ig, DUX4, FasL, H2-M3, HLA-C, HLA-E, HLA-E heavy chain, HLA-F, HLA-G, IDO1, IL-10, IL15-RF, IL-35, MANF, Mfge8, PD-L1, Serpinb9, and any combination thereof.

53. The modified SC-beta cell of embodiment 52, wherein the one or more additional tolerogenic factors comprise CD47.

54. The modified SC-beta cell of any one of embodiments 1-57, wherein the modified SC-beta cell further comprises one or more modifications that reduce expression of one or more additional molecules.

55. The modified SC-beta cell of embodiment 54, wherein the one or more additional molecules comprises B2M, TAP I, NLRC5, CIITA, HLA-A, HLA-B, HLA-C, HLA-DP, HLA-DQ, HLA-DR, HLA-DM, HLA-DO, RFX5, RFXANK, RFXAP, NFY-A, NFY-B, NFY-C, ABO, CADM1, CD58, CD38, CD142, CD155, CEACAM1, CTLA-4, FUT1, ICAM1, IRF1, MIC-A, MIC-B, NLGN4Y, PCDH11Y, PD-1, a protein that is involved in oxidative or ER stress, RHD, TRAC, TRB, optionally wherein the protein that is involved in oxidative or ER stress is selected from the group consisting of TXNIP, PERK, IRE1α, and DJ-1 (PARK7).

56. The modified SC-beta cell of embodiment 54 or 55, wherein the one or more additional molecules comprise one or more Y chromosome proteins, optionally Protocadherin-11 Y-linked (PCDH11Y) and/or Neuroligin-4 Y-linked (NLGN4Y).

57. The modified SC-beta cell of any of embodiments 54-57, wherein the one or more additional molecules comprise one or more NK cell ligands, optionally MIC-A and/or MIC-B.

58. The modified SC-beta cell of any of embodiments 54-57, wherein the one or more additional molecules comprise one or more proteins involved in oxidative or ER stress, optionally thioredoxin-interacting protein (TXNIP), PKR-like ER kinase (PERK), inositol-requiring enzyme 1a (IRE1α), and/or DJ-1 (PARK7).

59. The modified SC-beta cell of any of embodiments 54-58, wherein the one or more additional molecules comprise one or more blood antigen proteins, optionally ABO, FUT1 and/or RHD.

60. The modified SC-beta cell of any one of embodiments 1-59, wherein the modified SC-beta cell further comprises one or more modifications that reduce expression of B2M, TAP I, NLRC5, CIITA, HLA-A, HLA-B, HLA-C, HLA-DP, HLA-DQ, HLA-DR, HLA-DM, HLA-DO, RFX5, RFXANK, RFXAP, NFY-A, NFY-B, NFY-C, ABO, CADM1, CD58, CD38, CD142, CD155, CEACAM1, CTLA-4, FUT1, ICAM1, IRF1, MIC-A, MIC-B, NLGN4Y, PCDH11Y, PD-1, a protein that is involved in oxidative or ER stress, RHD, TRAC, TRB, optionally wherein the protein that is involved in oxidative or ER stress is selected from the group consisting of TXNIP, PERK, IRE1α, and DJ-1 (PARK7).

61. The modified SC-beta cell of embodiment 60, wherein TRB is TRBC1, TRBC2, or TRBC1 and TRBC2.

62. The modified SC-beta cell of any of embodiments 1-61, wherein reduced expression comprises no cell surface expression or no detectable cell surface expression.

63. The modified SC-beta cell of any of embodiments 1-62, wherein reduced expression comprises reduced mRNA expression, optionally wherein reduced expression comprises no detectable mRNA expression.

64. The modified SC-beta cell of any of embodiments 1-63, wherein reduced expression comprises reduced protein expression or reduced protein activity, optionally wherein reduced expression comprises no detectable protein expression or protein activity.

65. The modified SC-beta cell of any of embodiments 1-64, wherein reduced expression comprises eliminating activity of a gene encoding or regulating the expression of i) the one or more MHC class I molecules and/or the one or more MHC class II molecules, or ii) the one or more additional molecules.

66. The modified SC-beta cell of any of embodiments 1-65, wherein reduced expression comprises inactivation or disruption of an allele of a gene encoding or regulating the expression of i) the one or more MHC class I molecules and/or the one or more MHC class II molecules, or ii) the one or more additional molecules.

67. The modified SC-beta cell of any of embodiments 1-66, wherein reduced expression comprises inactivation or disruption of both alleles of a gene encoding or regulating the expression of i) the one or more MHC class I molecules and/or the one or more MHC class II molecules, or ii) the one or more additional molecules.

68. The modified SC-beta cell of any of embodiments 1-67, wherein the one or more modifications to reduce expression comprises an indel in a gene encoding or regulating the expression of i) the one or more MHC class I molecules and/or the one or more MHC class II molecules, or ii) the one or more additional molecules.

69. The modified SC-beta cell of any of embodiments 1-68, wherein the one or more modifications to reduce expression comprises a frameshift mutation or a deletion of a contiguous stretch of genomic DNA of a gene encoding or regulating the expression of i) the one or more MHC class I molecules and/or the one or more MHC class II molecules, or ii) the one or more additional molecules.

70. The modified SC-beta cell of any of embodiments 1-69, wherein the one or more modifications to reduce expression comprises inactivation or disruption of all coding sequences of a gene encoding or regulating the expression of i) the one or more MHC class I molecules and/or the one or more MHC class II molecules, or ii) the one or more additional molecules.

71. The modified SC-beta cell of any of embodiments 1-69, wherein the one or more modifications to reduce expression comprises knocking out a gene encoding or regulating the expression of i) the one or more MHC class I molecules and/or the one or more MHC class II molecules, or ii) the one or more additional molecules.

72. The modified SC-beta cell of any of embodiments 1-71, wherein the modified SC-beta cell comprises one or more modifications that:
a. reduce expression of MHC class I and/or MHC class II molecules;
b. increase expression of CD47; and
c. increase expression of CCL22.

73. The modified SC-beta cell of any of embodiments 1-71, wherein the modified SC-beta cell comprises one or more modifications that:
a. reduce expression of MHC class I and/or MHC class II molecules;
b. increase expression of CD47; and
c. increase expression of CD39.

74. The modified SC-beta cell of any of embodiments 1-71, wherein the modified SC-beta cell comprises one or more modifications that:
a. reduce expression of MHC class I and/or MHC class II molecules;
b. increase expression of CD47; and
c. increase expression of CD46 and CD59.

75. The modified SC-beta cell of any of embodiments 1-71, wherein the modified SC-beta cell comprises one or more modifications that:
a. reduce expression of MHC class I and/or MHC class II molecules;
b. increase expression of CD47; and
c. increase expression of PD-L1.

76. The modified SC-beta cell of any of embodiments 1-71, wherein the modified SC-beta cell comprises one or more modifications that:
a. reduce expression of MHC class I and/or MHC class II molecules;
b. increase expression of CD47; and
c. increase expression of HLA-G and PD-L1.

77. The modified SC-beta cell of any of embodiments 1-71, wherein the modified SC-beta cell comprises one or more modifications that:
a. reduce expression of MHC class I and/or MHC class II molecules;
b. increase expression of CD47; and
c. reduced expression of CD142 (TF).

78. The modified SC-beta cell of any of embodiments 1-71, wherein the modified SC-beta cell comprises one or more modifications that:
a. reduce expression of MHC class I and/or MHC class II molecules;
b. increase expression of CD47; and
c. reduced expression of MIC-A and/or MIC-B.

79. The modified SC-beta cell of any of embodiments 1-71, wherein the modified SC-beta cell comprises one or more modifications that:
a. reduce expression of MHC class I and/or MHC class II molecules; and
b. increase expression of CD24.

80. The modified SC-beta cell of any of embodiments 1-71, wherein the modified SC-beta cell comprises one or more modifications that:
a. reduce expression of MHC class I and/or MHC class II molecules; and
b. increase expression of CD200.

81. The modified SC-beta cell of any of embodiments 1-71, wherein the modified SC-beta cell comprises one or more modifications that:
a. reduce expression of MHC class I and/or MHC class II molecules; and
b. increase expression of CD52.

82. The modified SC-beta cell of any of embodiments 1-71, wherein the modified SC-beta cell comprises one or more modifications that:
a. reduce expression of MHC class I and/or MHC class II molecules; and
b. increase expression of DUX4.

83. The modified SC-beta cell of any of embodiments 1-71, wherein the modified SC-beta cell comprises one or more modifications that:
a. reduce expression of MHC class I and/or MHC class II molecules; and
b. increase expression of IDO1.

84. The modified SC-beta cell of any of embodiments 1-71, wherein the modified SC-beta cell comprises one or more modifications that:
a. reduce expression of MHC class I and/or MHC class II molecules; and
b. increase expression of IL-35.

85. The modified SC-beta cell of any of embodiments 1-71, wherein the modified SC-beta cell comprises one or more modifications that:
a. reduce expression of MHC class I and/or MHC class II molecules; and
b. increase expression of PD-L1.

86. The modified SC-beta cell of any of embodiments 1-71, wherein the modified SC-beta cell comprises one or more modifications that:
a. reduce expression of MHC class I and/or MHC class II molecules; and
b. increase expression of HLA-E.

87. The modified SC-beta cell of any of embodiments 1-71, wherein the modified SC-beta cell comprises one or more modifications that:
a. reduce expression of MHC class I and/or MHC class II molecules; and
b. increase expression of HLA-G.

88. The modified SC-beta cell of any of embodiments 1-71, wherein the modified SC-beta cell comprises one or more modifications that:
a. reduce expression of MHC class I and/or MHC class II molecules;
b. reduce expression of CD155; and
c. increase expression of HLA-E.

89. The modified SC-beta cell of any of embodiments 1-71, wherein the modified SC-beta cell comprises one or more modifications that:
a. reduce expression of MHC class I molecules;
b. reduce expression of RFXANK;
c. increase expression of HLA-E.

90. The modified SC-beta cell of any of embodiments 1-71, wherein the modified SC-beta cell comprises one or more modifications that:
a. reduce expression of MHC class I and/or MHC class II molecules;
b. reduce expression of MIC-A and/or MIC-B;
c. increase expression of one or more of CD47, CD24 and PD-L1; and
d. increase expression of CD46, CD55, CD59 and CR1.

91. The modified SC-beta cell of any of embodiments 1-71, wherein the modified SC-beta cell comprises one or more modifications that:
a. reduce expression of MHC class I molecules;
b. reduce expression of MIC-A and/or MIC-B;
c. reduce expression of TXNIP; and
d. increase expression of PD-L1 and HLA-E.

92. The modified SC-beta cell of embodiment 90, wherein the modifications further increase expression of A20/TNFAIP3 and MANF.

93. The modified SC-beta cell of any one of embodiments 2-92, wherein the one or more modifications that reduce expression of MHC class I and/or MHC class II molecules consist of one or more modifications that reduce expression of MHC class I molecules.

94. The modified SC-beta cell of any one of embodiments 2-92, wherein the one or more modifications that reduce expression of MHC class I and/or MHC class II molecules consist of one or more modifications that reduce expression of MHC class II molecules.

95. The modified SC-beta cell of any one of embodiments 2-92, wherein the one or more modifications that reduce expression of MHC class I and/or MHC class II molecules consist of one or more modifications that reduce expression of MHC class I molecules and MHC class II molecules.

96. The modified SC-beta cell of embodiment 1-95, wherein increased expression comprises increased mRNA expression.

97. The modified SC-beta cell of embodiment 1-96, wherein increased expression comprises increased protein expression or protein activity.

98. The modified SC-beta cell of any one of embodiments 1-97, wherein increased expression comprises increasing activity of a gene encoding or regulating the expression of i) the one or more tolerogenic factors, or ii) the one or more additional tolerogenic factors.

99. The modified SC-beta cell of embodiment 98, wherein the gene is an endogenous gene and the one or more modifications comprise one or more modifications of an endogenous promoter.

100. The modified SC-beta cell of embodiment 98, wherein the gene is an endogenous gene and the one or more modifications comprise introduction of a heterologous promoter.

101. The modified SC-beta cell of embodiment 100, wherein the heterologous promoter is selected from the group consisting of a CAG promoter, cytomegalovirus (CMV) promoter, EF1α promoter, EF1α short promoter, PGK promoter, adenovirus late promoter, vaccinia virus 7.5K promoter, SV40 promoter, tk promoter of HSV, mouse mammary tumor virus (MMTV) promoter, LTR promoter of HIV, promoter of moloney virus, Epstein Barr virus (EBV) promoter, and Rous sarcoma virus (RSV) promoter, and UBC promoter.

102. The modified SC-beta cell of any of embodiments 1-94, wherein the modified SC-beta cell comprises one or more transgenes.

103. The modified SC-beta cell of embodiment 102, wherein the one or more transgenes encode at least one of the one or more tolerogenic factors or the one or more additional tolerogenic factors.

104. The modified SC-beta cell of embodiment 102 or 103, wherein the one or more transgenes encode at least one of the one or more additional tolerogenic factors.

105. The modified SC-beta cell of any one of embodiments 102-104, wherein the one or more transgenes encode one or more additional molecules.

106. The modified SC-beta cell of any of embodiments 102-105, wherein the one or more transgenes comprise one or more regulatory elements.

107. The modified SC-beta cell of any of embodiments 102-106, wherein the one or more transgenes are operably linked to the one or more regulatory elements.

108. The modified SC-beta cell of embodiment 106 or embodiment 107, wherein the one or more regulatory elements comprise one or more promoters, enhancers, introns, terminators, translation initiation signals, polyadenylation signals, replication elements, RNA processing and export elements, transposons, transposases, insulators, internal ribosome entry sites (IRES), 5′UTRs, 3′UTRs, mRNA 3′ end processing sequences, boundary elements, locus control regions (LCR), matrix attachment regions (MAR), recombination or cassette exchange sequences, linker sequences, secretion signals, resistance markers, anchoring peptides, localization signals, fusion tags, affinity tags, chaperonins, and proteases.

109. The modified SC-beta cell of embodiment 106 or embodiment 108, wherein the one or more transgenes comprise two or more regulatory elements.

110. The modified SC-beta cell of any of embodiments 102-109, wherein the promoter is selected from the group consisting of a CAG promoter, cytomegalovirus (CMV) promoter, EF1α promoter, EF1α short promoter, PGK promoter, adenovirus late promoter, vaccinia virus 7.5K promoter, SV40 promoter, tk promoter of HSV, mouse mammary tumor virus (MMTV) promoter, LTR promoter of HIV, promoter of moloney virus, Epstein Barr virus (EBV) promoter, and Rous sarcoma virus (RSV) promoter, and UBC promoter.

111. The modified SC-beta cell of any of embodiments 102-110, wherein the modified SC-beta cell comprises one or more vectors encoding the one or more transgenes.

112. The modified SC-beta cell of embodiment 111, wherein at least one of the one or more vectors is a multicistronic vector.

113. The modified SC-beta cell of embodiment 112, wherein the multicistronic vector encodes at least one of the one or more tolerogenic factors or the one or more additional tolerogenic factors.

114. The modified SC-beta cell of embodiment 112 or embodiment 113, wherein the multicistronic vector further encodes at least one of the one or more tolerogenic factors or the one or more additional tolerogenic factors.

115. The modified SC-beta cell of embodiment of embodiment 113 or embodiment 114, wherein the multicistronic vector further encodes at least one of the one or more additional molecules.

116. The modified SC-beta cell of any one of embodiments 102-115, wherein the one or more transgenes are separated by one or more linker sequences.

117. The modified SC-beta cell of embodiment 116, wherein the one or more linker sequences comprise an IRES sequence or a cleavable peptide sequence.

118. The modified SC-beta cell of embodiment 117, wherein the cleavable peptide sequence comprises a self-cleavable peptide, optionally a 2A peptide.

119. The modified SC-beta cell of embodiment 118, wherein the 2A peptide is selected from the group consisting of a F2A sequence, an E2A sequence, a P2A sequence, and a T2A sequence.

120. The modified SC-beta cell of any of embodiments 117-119, wherein the cleavable peptide sequence comprises a protease cleavable sequence or a chemically cleavable sequence.

121. The modified SC-beta cell of any of embodiments 113-120, wherein at least two of the one or more tolerogenic factors, the one or more additional tolerogenic factors, and/or the one or more additional molecules are operably linked to the same promoter.

122. The modified SC-beta cell of embodiment 121, wherein the promoter is a constitutive promoter.

123. The modified SC-beta cell of embodiment 121 or 122, wherein the promoter is selected from the group consisting of a CAG promoter, cytomegalovirus (CMV) promoter, EF1α promoter, EF1α short promoter, PGK promoter, adenovirus late promoter, vaccinia virus 7.5K promoter, SV40 promoter, tk promoter of HSV, mouse mammary tumor virus (MMTV) promoter, LTR promoter of HIV, promoter of moloney virus, Epstein Barr virus (EBV) promoter, and Rous sarcoma virus (RSV) promoter, and UBC promoter.

124. The modified SC-beta cell of any of embodiments 105-123, wherein the one or more additional molecules comprise a chimeric antigen receptor (CAR).

125. The modified SC-beta cell of embodiment 124, wherein the CAR comprises a signal peptide, an extracellular binding domain specific to CD19, a hinge domain, a transmembrane domain, an intracellular costimulatory domain, and/or an intracellular signaling domain.

126. The modified SC-beta cell of embodiment 124 or embodiment 125, wherein the CAR is specific for CD19, CD20, CD22, CD38, CD123, CD138, BCMA, or any combination thereof.

127. The modified SC-beta cell of embodiment 126, wherein the CAR is a CD19/CD22-bispecific CAR.

128. The modified SC-beta cell of any of embodiments 105-127, wherein the one or more additional molecules comprise one or more safety switches.

129. The modified SC-beta cell of embodiment 128, wherein the one or more safety switches are capable of controlled killing of the modified SC-beta cell.

130. The modified SC-beta cell of embodiment 128 or 129, wherein the one or more safety switches induce controlled cell death in the presence of a drug or prodrug, or upon activation by a selective exogenous compound.

131. The modified SC-beta cell of any of embodiments 128-130, wherein the one or more safety switches comprise is an inducible protein capable of inducing apoptosis of the modified SC-beta cell.

132. The modified SC-beta cell of embodiment 131, wherein the inducible protein capable of inducing apoptosis of the modified SC-beta cell is a caspase protein.

133. The modified SC-beta cell of embodiment 132, wherein the caspase protein is caspase 9.

134. The modified SC-beta cell of any of embodiments 128-133, wherein the one or more safety switches comprise one or more suicide genes.

135. The modified SC-beta cell of embodiment 134, wherein the one or more suicide genes are selected from the group consisting of cytosine deaminase (CyD), herpesvirus thymidine kinase (HSV-Tk), an inducible caspase 9 (iCaspase9), and rapamycin-activated caspase 9 (rapaCasp9).

136. The modified SC-beta cell of any of embodiments 102-135, wherein at least one of the one or more transgenes are integrated into the genome of the modified SC-beta cell.

137. The modified SC-beta cell of embodiment 136, wherein integration is by non-targeted insertion into the genome of the modified SC-beta cell.

138. The modified SC-beta cell of embodiment 137, wherein integration is by non-targeted insertion into the genome of the modified SC-beta cell using a lentiviral vector.

139. The modified SC-beta cell of embodiment 136, wherein integration is by targeted insertion into a target genomic locus of the modified SC-beta cell.

140. The modified SC-beta cell of embodiment 139, wherein targeted insertion is by nuclease-mediated gene editing with homology-directed repair.

141. The modified SC-beta cell of embodiment 139 or 140, wherein the target genomic locus is selected from the group consisting of an albumin gene locus, an ABO gene locus, a B2M gene locus, a CIITA gene locus, a CCR5 gene locus, a CD142 gene locus, a CLYBL gene locus, a CXCR4 gene locus, an F3 gene locus, a FUT1 gene locus, an HMGB1 gene locus, a KDM5D gene locus, an LRP1 gene locus, a MIC-A gene locus, a MIC-B gene locus, a PPP1R12C (also known as AAVS1) gene locus, an RHD gene locus, a ROSA26 gene locus, a safe harbor gene locus, a SHS231 locus, a TAP1 gene locus, a TRAC gene locus, and a TRBC gene locus.

142. The modified SC-beta cell of any of embodiments 1-141, wherein the genome of the modified SC-beta cell comprises on or more gene edits in one or more genes encoding the one or more molecules of any of embodiments 1-141 having reduced expression.

143. The modified SC-beta cell of any of embodiments 1-142, wherein the modified SC-beta cell comprises a genome editing complex.

144. The modified SC-beta cell of embodiment 143, wherein the genome editing complex comprises a genome targeting entity and a genome modifying entity.

145. The modified SC-beta cell of embodiment 144, wherein the genome targeting entity localizes the genome editing complex to the target locus, optionally wherein the genome targeting entity is a nucleic acid-guided targeting entity.

146. The modified SC-beta cell of embodiment 144 or embodiment 145, wherein the genome targeting entity comprises a transcription activator-like effector (TALE) binding protein, a zinc finger (ZF) binding protein, a Meganuclease, a Cas protein, a TnpB protein, a homing endonuclease, an endonuclease-deficient-Cas protein, an enzymatically inactive Cas protein, a nucleic acid programmable DNA binding protein, or a functional portion thereof.

146a. The modified SC-beta cell of any of embodiments 144-146, wherein the genome targeting entity is selected from the group consisting of Cas1, Cas2, Cas3, Cas4, Cas5, Cas6, Cas7, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr1, Cmr2, Cmr3, Cmr4, Cmr5, Cmr6, Csd1, Csd2, Cas5d, Cse1, Cse2, Cse3, Cse4, Cas5e, Csf1, Csm1, Csm2, Csm3, Csm4, Csm5, Csn1, Csn2, Cst1, Cst2, Cas5t, Csh1, Csh2, Cas5h, Csa1, Csa2, Csa3, Csa4, Csa5, Cas5a, Csx10, Csx11, Csy1, Csy2, Csy3, Csy4, Mad7, SpCas9, eSpCas9, SpCas9-HF1, HypaSpCas9, HeFSpCas9, and evoSpCas9 high-fidelity variants of SpCas9, SaCas9, NmeCas9, CjCas9, StCas9, TdCas9, LbCas12a, AsCas12a, AacCas12b, BhCas12b v4, TnpB, dCas (D10A), dCas (H840A), dCas13a, dCas13b, a core Cas protein, a nucleic acid programmable DNA binding protein, an RNA guided nucleases, a homing endonuclease, a zinc finger nuclease (ZFN), a transcription activator-like effector nuclease (TALEN), a Meganuclease, a CRISPR-associated transposase, or a functional portion thereof.

147. The modified SC-beta cell of embodiment 144, wherein the genome modifying entity cleaves, deaminates, nicks, polymerizes, interrogates, integrates, cuts, unwinds, breaks, alters, methylates, demethylates, or otherwise destabilizes the target locus.

148. The modified SC-beta cell of embodiment 144 or embodiment 147, wherein the genome modifying entity comprises a recombinase, integrase, transposase, endonuclease, exonuclease, nickase, helicase, polymerase, reverse transcriptase, deaminase, flippase, methylase, demethylase, acetylase, a nucleic acid modifying protein, an RNA modifying protein, a DNA modifying protein, Argonaute protein, an epigenetic modifying protein, a histone modifying protein, or a functional portion thereof.

149. The modified SC-beta cell of embodiment 148, wherein the genome modifying entity is selected from the group consisting of Cas1, Cas2, Cas3, Cas4, Cas5, Cas6, Cas7, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr1, Cmr2, Cmr3, Cmr4, Cmr5, Cmr6, Csd1, Csd2, Cas5d, Cse1, Cse2, Cse3, Cse4, Cas5e, Csf1, Csm1, Csm2, Csm3, Csm4, Csm5, Csn1, Csn2, Cst1, Cst2, Cas5t, Csh1, Csh2, Cas5h, Csa1, Csa2, Csa3, Csa4, Csa5, Cas5a, Csx10, Csx11, Csy1, Csy2, Csy3, Csy4, Mad7, SpCas9, eSpCas9, SpCas9-HF1, HypaSpCas9, HeFSpCas9, and evoSpCas9 high-fidelity variants of SpCas9, SaCas9, NmeCas9, CjCas9, StCas9, TdCas9, LbCas12a, AsCas12a, AacCas12b, BhCas12b v4, TnpB, FokI, dCas (D10A), dCas (H840A), dCas13a, dCas13b, a core Cas protein, a nucleic acid programmable DNA binding protein, an RNA guided nucleases, a homing endonuclease, a zinc finger nuclease (ZFN), a transcription activator-like effector nuclease (TALEN), a Meganuclease, a CRISPR-associated transposase, APOBEC1, cytidine deaminase, adenosine deaminase, uracil glycosylase inhibitor (UGI), adenine base editors (ABE), cytosine base editors (CBE), reverse transcriptase, serine integrase, polymerase, adenine-to-thymine or “ATBE” (or thymine-to-adenine or “TABE”) transversion base editor, ten-eleven translocation methylcytosine dioxygenases (TETs), TET1, TET3, TET1CD, histone acetyltransferase p300, histone methyltransferase SMYD3, histone methyltransferase PRDM9, H3K79 methyltransferase DOT1L, transcriptional repressor, or a functional portion thereof.

150. The modified SC-beta cell of any of embodiments 144-149, wherein the genome targeting entity and the genome modifying entity are different domains of a single polypeptide.

151. The modified SC-beta cell of any of embodiments 144-150, wherein the genome editing entity and genome modifying entity are two different polypeptides that are operably linked together.

152. The modified SC-beta cell of any of embodiments 144-150, wherein the genome editing entity and genome modifying entity are two different polypeptides that are not linked together.

153. The modified SC-beta cell of any of embodiments 75-82, wherein the modification is by a genome-modifying protein.

154. The modified SC-beta cell of any of embodiments 83, wherein the modification by a genome-modifying protein is modification by a CRISPR-associated transposase, prime editing, or Programmable Addition via Site-specific Targeting Elements (PASTE).

155. The modified SC-beta cell of any of embodiments 83-84, wherein the modification by the genome-modifying protein is nuclease-mediated gene editing.

156. The modified SC-beta cell of embodiment 85, wherein the nuclease-mediated gene editing is by a zinc finger nuclease (ZFN), a TAL-effector nuclease (TALEN), or a CRISPR-Cas combination that targets the B2M gene, optionally wherein the Cas is selected from a Cas9 or a Cas12.

157. The modified SC-beta cell of any of embodiments 83-85, wherein the modification by the genome-modifying protein is performed by one or more proteins selected from the group consisting of Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, and a CRISPR-associated transposase.

158. The modified SC-beta cell of embodiment 86, wherein the nuclease-mediated gene editing is by a CRISPR-Cas combination and the CRISPR-Cas combination comprises a guide RNA (gRNA) having a targeting domain that is complementary to at least one target site within the B2M gene.

159. The modified SC-beta cell of embodiment 88, wherein the CRISPR-Cas combination is a ribonucleoprotein (RNP) complex comprising the gRNA and a Cas protein.

160. The modified SC-beta cell of any of embodiments 1-159, wherein the modified SC-beta cell is a human cell or an animal cell.

161. The modified SC-beta cell of embodiment 160, wherein the animal cell is a porcine cell, a bovine cell, or an ovine cell.

162. The modified SC-beta cell of embodiment 160, wherein the modified SC-beta cell is a human cell.

163. The modified SC-beta cell of any of embodiments 1-162, wherein the modified SC-beta cell is differentiated from a stem cell or progenitor cell.

164. The modified SC-beta cell of embodiment 163, wherein the modified SC-beta cell is a differentiated cell derived from the stem cell or progenitor cell, wherein the differentiation comprises:
(i) contacting the stem cell with a TGFbeta/Activin agonist and/or, a glycogen synthase kinase 3 (GSK) inhibitor and/or WNT agonist for an amount of time sufficient to form a definitive endoderm cell;
(ii) contacting a definitive endoderm cell differentiated from the stem cell with a FGFR2b agonist for an amount of time sufficient to form a primitive gut tube cell;
(iii) contacting a primitive gut tube cell differentiated from the stem cell with a retinoic acid receptor (RAR) agonist, a rho kinase inhibitor, a Smoothened antagonist, a FGFR2b agonist, a protein kinase C activator, and/or a BMP type 1 receptor inhibitor for an amount of time sufficient to form an early pancreas progenitor cell;
(iv) incubating an early pancreas progenitor cell differentiated from the stem cell for at least about 3 days and contacting the early pancreas progenitor cell with a rho kinase inhibitor, a TGFbeta-/Activin agonist, a Smoothened antagonist, an FGFR2b agonist, a RAR agonist, a protein kinase C activator, and/or a BMP type 1 receptor inhibitor for an amount of time sufficient to form a pancreatic progenitor cell, wherein the RAR agonist concentration is less than the RAR agonist concentration in step (iii);
(v) contacting a pancreatic progenitor cell differentiated from the stem cell with an Alk5 inhibitor/TGFbeta receptor inhibitor, a gamma secretase inhibitor, a Smoothened antagonist, an Erbb1 (EGFR) or Erbb4 agonist, a thyroid hormone, and/or a RAR agonist for an amount of time sufficient to form an endoderm cell, wherein during at least a portion of the contacting in (v) comprises depolymerizing the actin cytoskeleton at a time and for an amount of time sufficient to increase differentiation efficiency; and/or
(vi) incubating an endoderm cell differentiated from the stem cell for an amount of time in serum-free media sufficient to form a beta cell.

165. The modified SC-beta cell of embodiment 163 or 164, wherein the stem cell or progenitor cell is selected from the group consisting of an induced pluripotent stem cell, an embryonic stem cell, a pluripotent stem cell (PSC), and a multipotent stem cell.

166. The modified SC-beta cell of any of embodiments 1-162, wherein the modified SC-beta cell is a differentiated cell derived from a pluripotent stem cell or a progeny thereof.

167. The modified SC-beta cell of embodiment 166, wherein the pluripotent stem cell is an induced pluripotent stem cell.

168. The modified SC-beta cell of any of embodiments 1-162, wherein the modified SC-beta cell is a primary cell isolated from a donor subject.

169. The modified SC-beta cell of embodiment 168, wherein the donor subject is healthy at the time the donor sample is obtained from the individual donor.

170. The modified SC-beta cell of embodiment 168, wherein the donor subject is not suspected of having a disease or condition at the time the donor sample is obtained from the individual donor.

171. The modified SC-beta cell of any of embodiments 1-170, wherein the cell is ABO blood group type O.

172. The modified SC-beta cell of any of embodiments 1-171, wherein the cell comprises a functional ABO A allele and/or a functional ABO B allele.

173. The modified SC-beta cell of any of embodiments 1-172, wherein the cell is Rhesus factor negative (Rh−).

174. The modified SC-beta cell of any of embodiments 1-172, wherein the cell is Rhesus factor positive (Rh+).

175. A method of generating the modified SC-beta cell of any of embodiments 1-174 comprising
a. obtaining a cell; and
b. introducing the one or more modifications of any of embodiments 1-174 into the cell.

176. The method of embodiment 175, wherein the method further comprises selecting the modified SC-beta cell from a population of cells based on the presence and/or level of one or more of the modifications.

177. The method of embodiment 175 or 176, wherein the SC-beta cell is derived from a stem cell or a progenitor cell and the method further comprises differentiating the stem cell or the progenitor cell into the SC-beta cell.

178. The method of embodiment 175 or 176, wherein the SC-beta cell is derived from a pluripotent stem cell or a progeny thereof and the method comprises differentiating the pluripotent stem cell or progeny thereof into the SC-beta cell.

179. The method of embodiment 175 or 176, wherein the cell is a primary cell.

180. The method of any of embodiments 175-179, wherein the method comprises introducing one or more gene edits into the genome of the cell.

181. The method of embodiment 180, wherein the one or more gene edits are introduced into the genome of the cell by non-targeted insertion.

182. The method of embodiment 180, wherein the one or more gene edits are introduced into the genome of the cell by targeted insertion.

183. The method of embodiment 180 or 182, wherein the one or more gene edits are introduced into one or more genes encoding the one or more molecules of any of embodiments 1-173.

184. The method of embodiment 183, wherein the modified SC-beta cell has increased expression of the one or more molecules encoded by the one or more edited genes.

185. The method of embodiment 183 or 184, wherein the modified SC-beta cell has reduced expression of the one or more molecules encoded by the one or more edited genes.

186. The method of any of embodiments 180-185, wherein the one or more gene edits are introduced into the genome of cell using at least one of the genome editing complexes of any of embodiments 143-159.

187. The method of any of embodiments 180-186, wherein the one or more gene edits are introduced into the genome of cell at one or more target genomic loci selected from the group consisting of an albumin gene locus, an ABO gene locus, a B2M gene locus, a CIITA gene locus, a CCR5 gene locus, a CD142 gene locus, a CLYBL gene locus, a CXCR4 gene locus, an F3 gene locus, a FUT1 gene locus, an HMGB1 gene locus, a KDM5D gene locus, an LRP1 gene locus, a MIC-A gene locus, a MIC-B gene locus, a PPP1R12C (also known as AAVS1) gene locus, an RHD gene locus, a ROSA26 gene locus, a safe harbor gene locus, a SHS231 locus, a TAP1 gene locus, a TRAC gene locus, and a TRBC gene locus.

188. A modified SC-beta cell produced according to the method of any of embodiments 175-187.

189. The modified SC-beta cell of any of embodiments 1-174 and 188, wherein the modified SC-beta cell has increased capability to evade NK cell mediated cytotoxicity upon administration to a subject as compared to a cell of the same type that does not comprise the one or more modifications.

190. The modified SC-beta cell of any of embodiments 1-174, 188 and 189, wherein the modified SC-beta cell undergoes reduced cell lysis by mature NK cells upon administration to a subject as compared to a cell of the same type that does not comprise the one or more modifications.

191. The modified SC-beta cell of any of embodiments 1-174 and 188-190, wherein the modified SC-beta cell induces a reduced immune response upon administration to a subject as compared to a cell of the same type that does not comprise the one or more modifications.

192. The modified SC-beta cell of any of embodiments 1-174 and 188-191, wherein the modified SC-beta cell induces a reduced systemic inflammatory response upon administration to a subject as compared to a cell of the same type that does not comprise the one or more modifications.

193. The modified SC-beta cell of any of embodiments 1-174 and 188-192, wherein the modified SC-beta cell induces a reduced local inflammatory response upon administration to a subject as compared to a cell of the same type that does not comprise the one or more modifications.

194. The modified SC-beta cell of any of claims 1-174 and 188-193, wherein the modified SC-beta cell reduced complement pathway activation upon administration to a subject as compared to a cell of the same type that does not comprise the one or more modifications.

195. The modified SC-beta cell of any of embodiments 1-174 and 188-194, wherein the modified SC-beta cell retains the ability to engraft and function upon administration to a subject.

196. The modified SC-beta cell of any of embodiments 1-174 and 188-195, wherein the modified SC-beta cell has increased ability to engraft and function upon administration to a subject as compared to a cell of the same type that does not comprise the one or more modifications.

197. A population of modified SC-beta cells comprising a plurality of the modified SC-beta cells of any of embodiments 1-174 and 188-196.

198. The population of modified SC-beta cells of embodiment 197, wherein at least about 30% of cells in the population comprise the plurality of the modified SC-beta cells.

199. The population of modified SC-beta cells of embodiment 197 or embodiment 198, wherein the plurality of the modified SC-beta cells are primary cells isolated from more than one donor subject.

200. The population of modified SC-beta cells of embodiment 199, wherein each donor subject is healthy or is not suspected of having a disease or condition at the time the donor sample is obtained from the individual donor.

201. A method of producing a composition comprising the modified SC-beta cell of any of embodiments 1-174 and 188-196 or the population of modified SC-beta cells of any of embodiments 197-200 comprising
a. obtaining the cell of any of embodiments 160-174;
b. introducing the one or more modifications of any of embodiments 1-174 into the cell
c. selecting the modified SC-beta cell or selecting the population of modified SC-beta cells from a population of cells based on a level of the one or more of the modifications; and
d. formulating the composition comprising the selected modified SC-beta cell or the selected population of modified SC-beta cells.

202. The method of embodiment 201, wherein method comprises selecting the modified SC-beta cell or the population of modified SC-beta cells based on the level of cell surface expression of the one or more modified molecules in any of embodiments 1-173.

203. The method of embodiment 201 or embodiment 202, wherein the modified SC-beta cell or the population of modified SC-beta cells are selected based on a level of the one or more modified molecules having reduced expression in the modified SC-beta cell or the population of modified SC-beta cells.

204. The method of any of embodiments 201-203, wherein the modified SC-beta cell or the population of modified SC-beta cells are selected based on a level of the one or more modified molecules having increased expression in the modified SC-beta cell or the population of modified SC-beta cells.

205. The method of any of embodiments 201-204, wherein the method comprises formulating the composition in a pharmaceutically acceptable additive, carrier, diluent, or excipient.

206. The method of embodiment 205, wherein the pharmaceutically acceptable additive, carrier, diluent, or excipient comprises a pharmaceutically acceptable buffer.

207. The method of embodiment 206, wherein the pharmaceutically acceptable buffer comprises neutral buffer saline or phosphate buffered saline.

208. The method of any of embodiments 201-207, wherein the method comprises formulating the composition with Plasma-Lyte A®, dextrose, dextran, sodium chloride, human serum albumin (HSA), dimethylsulfoxide (DMSO), or a combination thereof.

209. The method of any of embodiments 201-208, wherein the method comprises formulating the composition with a cryoprotectant.

210. The method of any of embodiments 201-209, wherein the method comprises formulating the composition in a serum-free cryopreservation medium comprising a cryoprotectant.

211. The method of embodiment 209 or embodiment 210, wherein the cryoprotectant comprises DMSO.

212. The method of embodiment 210 or embodiment 211, wherein the serum-free cryopreservation medium comprises about 5% to about 10% DMSO (v/v).

213. The method of any of embodiments 210-212, wherein the serum-free cryopreservation medium comprises about 10% DMSO (v/v).

214. The method of any of embodiments 201-213, wherein the method further comprises storing the composition in a container.

215. The method of any of embodiments 201-214, wherein the method further comprises thawing the cell before step (b).

216. The method of any of embodiments 201-215, wherein the method further comprises freezing the modified SC-beta cell, the population of modified SC-beta cells, or the composition.

217. The method of embodiment 216, wherein the modified SC-beta cell or the population of modified SC-beta cells are frozen after step (b).

218. The method of embodiment 217, wherein the modified SC-beta cell or the population of modified SC-beta cells are thawed before step (c).

219. The method of embodiment 216, wherein the modified SC-beta cell or the population of modified SC-beta cells are frozen after step (c).

220. The method of embodiment 219, wherein the modified SC-beta cell or the population of modified SC-beta cells are thawed before step (d).

221. The method of embodiment 216, wherein the modified SC-beta cell or the population of modified SC-beta cells are frozen after step (c).

222. The method of any of embodiments 201-221, wherein the composition is frozen after step (d).

223. A composition comprising the modified SC-beta cell of any of embodiments 1-174 and 188-196 or the population of modified SC-beta cells of any of embodiments 197-200.

224. A composition produced by the method of any one of embodiments 201-222.

225. The composition of embodiment 223 or embodiment 224, wherein the composition comprises a pharmaceutically acceptable additive, carrier, diluent, or excipient.

226. The composition of any of embodiments 223-225, wherein the composition is sterile.

227. A container comprising the composition of any of embodiments 224-226.

228. The container of embodiment 227, wherein the container is a sterile bag.

229. The container of embodiment 228, wherein the sterile bag is a cryopreservation-compatible bag.

230. A kit comprising the composition of any of embodiments 224-226 or the container of any of embodiments 227-229.

231. The kit of embodiment 230, wherein the kit further comprises instructions for using the modified SC-beta cells or the population of modified SC-beta cells.

232. A method of treating a condition or disease in a subject in need thereof comprising administering to the subject an effective amount of the modified SC-beta cell of any of embodiments 1-174 and 188-196, the population of modified SC-beta cells of any of embodiments 197-200, or the composition of any of embodiments 223-225, optionally wherein the disease or condition is a cellular deficiency.

234. The method of embodiment 232, wherein the condition or disease is associated with diabetes or is diabetes, optionally wherein the diabetes is Type I diabetes.

235. The method of any of embodiments 232-234, wherein the modified SC-beta cell or the population of modified SC-beta cells are expanded and cryopreserved prior to administration.

236. The method of any of embodiments 232-235, wherein the method comprises intravenous injection, intramuscular injection, intravascular injection, or transplantation of the modified SC-beta cell, the population of modified SC-beta cells, or the composition.

237. The method of embodiment 236, wherein transplantation comprises intravascular injection or intramuscular injection.

238. The method of any of embodiments 232-237, wherein the method further comprises administering one or more immunosuppressive agents to the subject.

239. The method of any of embodiments 232-238, wherein the subject has been administered one or more immunosuppressive agents.

240. The method of embodiment 238 or embodiment 239, wherein the one or more immunosuppressive agents are a small molecule or an antibody.

241. The method of any of embodiments 238-240, wherein the one or more immunosuppressive agents are selected from the group consisting of cyclosporine, azathioprine, mycophenolic acid, mycophenolate mofetil, a corticosteroids, prednisone, methotrexate, gold salts, sulfasalazine, antimalarials, brequinar, leflunomide, mizoribine, 15-deoxyspergualine, 6-mercaptopurine, cyclophosphamide, rapamycin, tacrolimus (FK-506), OKT3, anti-thymocyte globulin, thymopentin (thymosin-α), an immunomodulatory agent, and an immunosuppressive antibody.

242. The method of any of embodiments 238-241, wherein the one or more immunosuppressive agents comprise cyclosporine.

243. The method of any of embodiments 238-241, wherein the one or more immunosuppressive agents comprise mycophenolate mofetil.

244. The method of any of embodiments 238-241, wherein the one or more immunosuppressive agents comprise a corticosteroid.

245. The method of any of embodiments 238-241, wherein the one or more immunosuppressive agents comprise cyclophosphamide.

246. The method of any of embodiments 238-241, wherein the one or more immunosuppressive agents comprise rapamycin.

247. The method of any of embodiments 238-241, wherein the one or more immunosuppressive agents comprise tacrolimus (FK-506).

248. The method of any of embodiments 238-241, wherein the one or more immunosuppressive agents comprise anti-thymocyte globulin.

249. The method of any of embodiments 238-241, wherein the one or more immunosuppressive agents are one or more immunomodulatory agents.

250. The method of embodiment 249, wherein the one or more immunomodulatory agents are a small molecule or an antibody.

251. The method of embodiment 249 or embodiment 250, wherein the antibody binds to one or more receptors or ligands selected from the group consisting of p75 of the IL-2 receptor, MHC, CD2, CD3, CD4, CD7, CD28, B7, CD40, CD45, IFN-gamma, TNF-alpha, IL-4, IL-5, IL-6R, IL-6, IGF, IGFR1, IL-7, IL-8, IL-10, CD11a, CD58, and antibodies binding to any of their ligands.

252. The method of any of embodiments 238-251, wherein the one or more immunosuppressive agents are or have been administered to the subject prior to administration of the modified SC-beta cell, the population of modified SC-beta cells, or the composition.

253. The method of any of embodiments 238-252, wherein the one or more immunosuppressive agents are or have been administered to the subject at least 1, 2, 3, 4, 5, 6, 7, 8, 9, 10, 11, 12, 13, or 14 days prior to administration of the modified SC-beta cell, the population of modified SC-beta cells, or the composition.

254. The method of any of embodiments 238-253, wherein the one or more immunosuppressive agents are or have been administered to the subject at least 1 week, 2 weeks, 3 weeks, 4 weeks, 5 weeks, 6 weeks, 7 weeks, 8 weeks, 9 weeks, 10 weeks or more prior to administration of the modified SC-beta cell, the population of modified SC-beta cells, or the composition.

255. The method of any of embodiments 238-251, wherein the one or more immunosuppressive agents are or have been administered to the subject after administration of the modified SC-beta cell, the population of modified SC-beta cells, or the composition.

256. The method of any of embodiments 238-251 and 255, wherein the one or more immunosuppressive agents are or have been administered to the subject at least 1, 2, 3, 4, 5, 6, 7, 8, 9, 10, 11, 12, 13, or 14 days after administration of the modified SC-beta cell, the population of modified SC-beta cells, or the composition.

257. The method of any of embodiments 238-251, 255 and 256, wherein the one or more immunosuppressive agents are or have been administered to the subject at least 1 week, 2 weeks, 3 weeks, 4 weeks, 5 weeks, 6 weeks, 7 weeks, 8 weeks, 9 weeks, 10 weeks, or more, after administration of the modified SC-beta cell, the population of modified SC-beta cells, or the composition.

258. The method of any of embodiments 238-251, wherein the one or more immunosuppressive agents are or have been administered to the subject on the same day as the first administration of the modified SC-beta cell, the population of modified SC-beta cells, or the composition.

259. The method of any of embodiments 238-251, wherein the one or more immunosuppressive agents are or have been administered to the subject after administration of a first and/or second administration of the modified SC-beta cell, the population of modified SC-beta cells, or the composition.

260. The method of any of embodiments 238-251, wherein the one or more immunosuppressive agents are or have been administered to the subject prior to administration of a first and/or second administration of the modified SC-beta cell, the population of modified SC-beta cells, or the composition.

261. The method of any of embodiments 238-251, wherein the one or more immunosuppressive agents are or have been administered to the subject at least 1, 2, 3, 4, 5, 6, 7, 8, 9, 10, 11, 12, 13, or 14 days prior to administration of a first and/or second administration of the modified SC-beta cell, the population of modified SC-beta cells, or the composition.

262. The method of any of embodiments 238-251, wherein the one or more immunosuppressive agents are or have been administered to the subject at least 1 week, 2 weeks, 3 weeks, 4 weeks, 5 weeks, 6 weeks, 7 weeks, 8 weeks, 9 weeks, 10 weeks or more prior to administration of a first and/or second administration of the modified SC-beta cell, the population of modified SC-beta cells, or the composition.

263. The method of any of embodiments 238-251, wherein the one or more immunosuppressive agents are or have been administered to the subject at least 1, 2, 3, 4, 5, 6, 7, 8, 9, 10, 11, 12, 13, or 14 days after administration of a first and/or second administration of the modified SC-beta cell, the population of modified SC-beta cells, or the composition.

264. The method of any of embodiments 238-251, wherein the one or more immunosuppressive agents are or have been administered to the subject at least 1 week, 2 weeks, 3 weeks, 4 weeks, 5 weeks, 6 weeks, 7 weeks, 8 weeks, 9 weeks, 10 weeks, or more, after administration of a first and/or second administration of the modified SC-beta cell, the population of modified SC-beta cells, or the composition.

265. The method of any of embodiments 238-264, wherein the one or more immunosuppressive agents are administered at a lower dosage as compared to the dosage administered to reduce immune rejection of a cell that does not comprise the one or more modifications of the modified SC-beta cell or the population of modified SC-beta cells.

266. The method of any of embodiments 232-265, wherein the method further comprises activating the safety switch to induce controlled cell death after the administration of the the modified SC-beta cell, the population of modified SC-beta cells, or the composition to the subject.

267. The method of any of embodiments 232-266, wherein the suicide gene or the suicide switch is activated to induce controlled cell death after the administration of the one or more immunosuppressive agents to the subject.

268. The method of any of embodiments 232-266, wherein the suicide gene or the suicide switch is activated to induce controlled cell death prior to the administration of the one or more immunosuppressive agents to the subject.

269. The method of any of embodiments 232-268, wherein the safety switch is activated to induce controlled cell death in the event of cytotoxicity or other negative consequences to the subject.

270. The method of any of embodiments 232-269, wherein the method comprises administering an agent that allows for depletion of the modified SC-beta cell, the population of modified SC-beta cells, or the composition.

271. The method of embodiment 270, wherein the agent that allows for depletion of the modified SC-beta cell is an antibody that recognizes a protein expressed on the cell surface.

272. The method of embodiment 271, wherein the antibody is selected from the group consisting of an antibody that recognizes CCR4, CD16, CD19, CD20, CD30, EGFR, GD2, HER1, HER2, MUC1, PSMA, and RQR8.

273. The method of embodiment 271 or embodiment 272, wherein the antibody is selected from the group consisting of mogamulizumab, AFM13, MOR208, obinutuzumab, ublituximab, ocaratuzumab, rituximab, rituximab-Rllb, tomuzotuximab, RO5083945 (GA201), cetuximab, Hul4.18K322A, Hul4.18-IL2, Hu3F8, dinituximab, c.60C3-Rllc, and biosimilars thereof.

274. The method of any of embodiments 232-271, wherein the method comprises administering an agent that recognizes the one or more tolerogenic factors or the one or more additional tolerogenic factors on the cell surface.

275. The method of any of embodiments 232-274, wherein the method further comprises administering one or more additional therapeutic agents to the subject.

276. The method of any of embodiments 232-274, wherein the subject has been administered one or more additional therapeutic agents.

277. The method of any of embodiments 232-276, wherein the method further comprises monitoring the therapeutic efficacy of the method.

278. The method of any of embodiments 232-277, further comprising monitoring the prophylactic efficacy of the method.

279. The method of embodiment 277 or embodiment 278, wherein the method is repeated until a desired suppression of one or more disease symptoms occurs.

### VI. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The term “about” as used herein when referring to a measurable value, such as an amount or concentration and the like, is meant to encompass variations of 20%, 10%, 5%, 1%, 0.5%, or even 0.1% of the specified amount. As used herein, including in the appended claims, the singular forms “a,” “or,” and “the” include plural referents unless the context clearly dictates otherwise. For example, “a” or “an” means “at least one” or “one or more.” It is understood that aspects and variations described herein include embodiments “consisting” and/or “consisting essentially of” such aspects and variations.

As used herein, the term “and/or” includes any and all combinations of one or more of the associated listed items.

As used herein, the term “exogenous” with reference to a polypeptide or a polynucleotide is intended to mean that the referenced molecule is introduced into the cell of interest. The exogenous molecule, such as exogenous polynucleotide, can be introduced, for example, by introduction of an exogenous encoding nucleic acid into the genetic material of the cells such as by integration into a chromosome or as non-chromosomal genetic material such as a plasmid or expression vector. Therefore, the term as it is used in reference to expression of an encoding nucleic acid refers to introduction of the encoding nucleic acid in an expressible form into the cell. In some cases, an “exogenous” molecule is a molecule, construct, factor and the like that is not normally present in a cell, but can be introduced into a cell by one or more genetic, biochemical or other methods.

The term “endogenous” refers to a referenced molecule, such as a polynucleotide (e.g. gene), or polypeptide, that is present in a native or unmodified cell. For instance, the term when used in reference to expression of an endogenous gene refers to expression of a gene encoded by an endogenous nucleic acid contained within the cell and not exogenously introduced. A “gene,” includes a DNA region encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions. The sequence of a gene is typically present at a fixed chromosomal position or locus on a chromosome in the cell.

The term “locus” refers to a fixed position on a chromosome where a particular gene or genetic marker is located. Reference to a “target locus” refers to a particular locus of a desired gene in which it is desired to target a genetic modification, such as a gene edit or integration of an exogenous polynucleotide.

The term “expression” with reference to a gene or “gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or can be a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristoylation, and glycosylation. Hence, reference to expression or gene expression includes protein (or polypeptide) expression or expression of a transcribable product of or a gene such as mRNA. The protein expression may include intracellular expression or surface expression of a protein. Typically, expression of a gene product, such as mRNA or protein, is at a level that is detectable in the cell.

As used herein, a “detectable” expression level, means a level that is detectable by standard techniques known to a skilled artisan, and include for example, differential display, RT (reverse transcriptase)-coupled polymerase chain reaction (PCR), Northern Blot, and/or RNase protection analyses as well as immunoaffinity-based methods for protein detection, such as flow cytometry, ELISA, or western blot. The degree of expression levels need only be large enough to be visualized or measured via standard characterization techniques.

As used herein, the term “differentiation” or “differentiated” refers to a process by which an unspecialized (“uncommitted”) or less specialized cell acquires the features of a specialized cell such as, for example, a pancreatic cell. A differentiated cell is one that has taken on a more specialized (“committed”) position within the lineage of a cell. The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. As used herein, the lineage of a cell defines the heredity of the cell, i.e., which cells it came from and to what cells it can give rise. The lineage of a cell places the cell within a hereditary scheme of development and differentiation. A lineage-specific marker refers to a characteristic specifically associated with the phenotype of cells of a lineage of interest and can be used to assess the differentiation of an uncommitted cell to the lineage of interest.

As used herein, the term “increased expression”, “enhanced expression” or “overexpression” means any form of expression that is additional to the expression in an original or source cell that does not contain the modification for modulating a particular gene expression, for instance a wild-type expression level (which can be absence of expression or immeasurable expression as well). Reference herein to “increased expression,” “enhanced expression” or “overexpression” is taken to mean an increase in gene expression and/or, as far as referring to polypeptides, increased polypeptide levels and/or increased polypeptide activity, relative to the level in a cell that does not contain the modification, such as the original source cell prior to the engineering to introduce the modification, such as an unmodified cell or a wild-type cell. The increase in expression, polypeptide levels or polypeptide activity can be at least 5%, 10%, 20%, 30%, 40% or 50%, 60%, 70%, 80%, 85%, 90%, or 100% or even more. In some cases, the increase in expression, polypeptide levels or polypeptide activity can be at least 2-fold, 5-fold, 10-fold, 20-fold, 30-fold, 40-fold, 50-fold, 60-fold, 70-fold, 80-fold, 90-fold, 100-fold, 200-fold or more.

The term “hypoimmunogenic” refers to a cell that is less prone to immune rejection by a subject to which such cells are transplanted. For example, relative to a similar cell that does not contain modifications, such as an unaltered or unmodified wild-type cell, such a hypoimmunogenic cell may be about 2.5%, 5%, 10%, 20%, 30%, 40%, 50%, 60%, 70%, 80%, 90%, 95%, 97.5%, 99% or more less prone to immune rejection by a subject into which such cells are transplanted. Typically, the hypoimmunogenic cells are allogenic to the subject and a hypoimmunogenic cell evades immune rejection in an MHC-mismatched allogeneic recipient. In some embodiments, a hypoimmunogenic cell is protected from T cell-mediated adaptive immune rejection and/or innate immune cell rejection.

Hypoimmunogenicity of a cell can be determined by evaluating the immunogenicity of the cell such as the cell's ability to elicit adaptive and/or innate immune responses. Such immune response can be measured using assays recognized by those skilled in the art.

The term “tolerogenic factor” as used herein include immunosuppressive factors or immune-regulatory factors that modulate or affect the ability of a cell to be recognized by the immune system of a host or recipient subject upon administration, transplantation, or engraftment. Typically a tolerogenic factor is a factor that induces immunological tolerance to a modified primary cell so that the modified primary cell is not targeted, such as rejected, by the host immune system of a recipient. Hence, a tolerogenic factor may be a hypoimmunity factor. Examples of tolerogenic factors include immune cell inhibitory receptors (e.g. CD47), proteins that engage immune cell inhibitory receptors, checkpoint inhibitors and other molecules that reduce innate or adaptive immune recognition

The terms “decrease,” “reduced,” “reduction,” and “decrease” are all used herein generally to mean a decrease by a statistically significant amount. However, for avoidance of doubt, decrease,” “reduced,” “reduction,” “decrease” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The terms “increased”, “increase” or “enhance” or “activate” are all used herein to generally mean an increase by a statically significant amount; for the avoidance of any doubt, the terms “increased”, “increase” or “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

As used herein, the term “modification” with reference to a cell refers to any change or alteration of a nucleic acid in the genome of a cell, which may impact gene expression in the cell. For example, a modification includes a genetic modification that results in alterations, additions, and/or deletion of genes or portions of genes or other nucleic acid sequences. A modified cell, such as a genetically modified cell, can also refer to a cell with an added, deleted and/or altered gene or portion of a gene. In some embodiments, the modification is a genetic modification that directly changes the gene or regulatory elements thereof encoding a protein product in a cell, such as by gene editing, mutagenesis or by genetic engineering of an exogenous polynucleotide or transgene. Genetic modifications include, for example, both transient knock-in or knock-down mechanisms, and mechanisms that result in permanent knock-in, knock-down, or knock-out of target genes or portions of genes or nucleic acid sequences Genetic modifications include, for example, both transient knock-in and mechanisms that result in permanent knock-in of nucleic acids sequences Genetic modifications also include, for example, reduced or increased transcription, reduced or increased mRNA stability, reduced or increased translation, and reduced or increased protein stability.

As used herein, “indel” refers to a mutation resulting from an insertion, deletion, or a combination thereof, of nucleotide bases in the genome. Thus, an indel typically inserts or deletes nucleotides from a sequence. As will be appreciated by those skilled in the art, an indel in a coding region of a genomic sequence will result in a frameshift mutation, unless the length of the indel is a multiple of three. A CRISPR/Cas system of the present disclosure can be used to induce an indel of any length in a target polynucleotide sequence.

In some embodiments, the alteration is a point mutation. As used herein, “point mutation” refers to a substitution that replaces one of the nucleotides. A CRISPR/Cas system of the present disclosure can be used to induce an indel of any length or a point mutation in a target polynucleotide sequence.

As used herein, “knock out” includes deleting all or a portion of the target polynucleotide sequence in a way that interferes with the function of the target polynucleotide sequence. For example, a knock out can be achieved by altering a target polynucleotide sequence by inducing an indel in the target polynucleotide sequence in a functional domain of the target polynucleotide sequence (e.g., a DNA binding domain). Those skilled in the art will readily appreciate how to use the CRISPR/Cas systems of the present disclosure to knock out a target polynucleotide sequence or a portion thereof based upon the details described herein.

In some embodiments, the alteration results in a knock out of the target polynucleotide sequence or a portion thereof. Knocking out a target polynucleotide sequence or a portion thereof using a CRISPR/Cas system of the present disclosure can be useful for a variety of applications. For example, knocking out a target polynucleotide sequence in a cell can be performed in vitro for research purposes. For ex vivo purposes, knocking out a target polynucleotide sequence in a cell can be useful for treating or preventing a disorder associated with expression of the target polynucleotide sequence (e.g., by knocking out a mutant allele in a cell ex vivo and introducing those cells comprising the knocked out mutant allele into a subject).

By “knock in” herein is meant a process that adds a genetic function to a host cell. This causes increased levels of the knocked in gene product, e.g., an RNA or encoded protein. As will be appreciated by those in the art, this can be accomplished in several ways, including adding one or more additional copies of the gene to the host cell or altering a regulatory component of the endogenous gene increasing expression of the protein is made. This may be accomplished by modifying the promoter, adding a different promoter, adding an enhancer, or modifying other gene expression sequences.

In some embodiments, an alteration or modification described herein results in reduced expression of a target or selected polynucleotide sequence. In some embodiments, an alteration or modification described herein results in reduced expression of a target or selected polypeptide sequence.

In some embodiments, an alteration or modification described herein results in increased expression of a target or selected polynucleotide sequence. In some embodiments, an alteration or modification described herein results in increased expression of a target or selected polypeptide sequence.

“Modulation” of gene expression refers to a change in the expression level of a gene. Modulation of expression can include, but is not limited to, gene activation and gene repression. Modulation may also be complete, i.e, wherein gene expression is totally inactivated or is activated to wildtype levels or beyond; or it may be partial, wherein gene expression is partially reduced, or partially activated to some fraction of wildtype levels.

The term “operatively linked” or “operably linked” are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence, but need not be directly adjacent to it. For example, an enhancer is a transcriptional regulatory sequence that is operatively linked to a coding sequence, even though they are not contiguous.

As used herein, “pluripotent stem cells” have the potential to differentiate into any of the three germ layers: endoderm (e.g., the stomach linking, gastrointestinal tract, lungs, etc.), mesoderm (e.g., muscle, bone, blood, urogenital tissue, etc.) or ectoderm (e.g., epidermal tissues and nervous system tissues). The term “pluripotent stem cells,” as used herein, also encompasses “induced pluripotent stem cells”, or “iPSCs”, or a type of pluripotent stem cell derived from a non-pluripotent cell. In some embodiments, a pluripotent stem cell is produced or generated from a cell that is not a pluripotent cell. In other words, pluripotent stem cells can be direct or indirect progeny of a non-pluripotent cell. Examples of parent cells include somatic cells that have been reprogrammed to induce a pluripotent, undifferentiated phenotype by various means. Such “iPS” or “iPSC” cells can be created by inducing the expression of certain regulatory genes or by the exogenous application of certain proteins. Methods for the induction of iPS cells are known in the art and are further described below. (See, e.g., Zhou et al., Stem Cells 27 (11): 2667-74 (2009); Huangfu et al., Nature Biotechnol. 26 (7): 795 (2008); Woltjen et al., Nature 458 (7239): 766-770 (2009); and Zhou et al., Cell Stem Cell 8:381-384 (2009); each of which is incorporated by reference herein in their entirety.) As used herein, “hiPSCs” are human induced pluripotent stem cells. In some embodiments, “pluripotent stem cells,” as used herein, also encompasses mesenchymal stem cells (MSCs), and/or embryonic stem cells (ESCs).

The terms “polypeptide” and “protein,” as used herein, may be used interchangeably to refer to a series of amino acid residues joined by peptide bonds (i.e. a polymer of amino acid residues), and are not limited to a minimum length. Such polymers may contain natural or non-natural amino acid residues, or combinations thereof, and include, but are not limited to, peptides, polypeptides, oligopeptides, dimers, trimers, and multimers of amino acid residues. Thus, a protein or polypeptide includes include those with modified amino acids (e.g., phosphorylated, glycated, glycosylated, etc.) and amino acid analogs. Full-length polypeptides or proteins, and fragments thereof, are encompassed by this definition. The terms also include modified species thereof, e.g., post-translational modifications of one or more residues, for example, methylation, phosphorylation glycosylation, sialylation, or acetylation.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For instance, where a range of values is provided, it is understood that each intervening value, to the tenth of the unit of the lower limit, unless the context clearly dictate otherwise, between the upper and lower limit of that range and any other stated or intervening value in that stated range, is encompassed within the disclosure, subject to any specifically excluded limit in the stated range. Where the stated range includes one or both of the limits, ranges excluding either or both of those included limits are also included in the disclosure. In some embodiments, two opposing and open-ended ranges are provided for a feature, and in such description it is envisioned that combinations of those two ranges are provided herein. For example, in some embodiments, it is described that a feature is greater than about 10 units, and it is described (such as in another sentence) that the feature is less than about 20 units, and thus, the range of about 10 units to about 20 units is described herein.

As used herein, “safe harbor locus” refers to a gene locus that allows expression of a transgene or an exogenous gene in a manner that enables the newly inserted genetic elements to function predictably and that also may not cause alterations of the host genome in a manner that poses a risk to the host cell. Exemplary “safe harbor” loci include, but are not limited to, a CCR5 gene, a PPP1R12C (also known as AAVS1) gene, a CLYBL gene, and/or a Rosa gene (e.g., ROSA26).

As used herein, a “target locus” refers to a gene locus that allows expression of a transgene or an exogenous gene. Exemplary “target loci” include, but are not limited to, a CXCR4 gene, an albumin gene, a SHS231 locus, an F3 gene (also known as CD142), a MICA gene, a MICB gene, a LRP1 gene (also known as CD91), a HMGB1 gene, an ABO gene, a RHD gene, a FUT1 gene, and/or a KDM5D gene (also known as HY). The exogenous polynucleotide encoding the exogenous gene can be inserted in the CDS region for B2M, CIITA, TRAC, TRBC, CCR5, F3 (i.e., CD142), MICA, MICB, LRP1, HMGB1, ABO, RHD, FUT1, KDM5D (i.e., HY), PDGFRa, OLIG2, and/or GFAP. The exogenous polynucleotide encoding the exogenous gene can be inserted in introns 1 or 2 for PPP1R12C (i.e., AAVS1) or CCR5. The exogenous polynucleotide encoding the exogenous gene can be inserted in exons 1 or 2 or 3 for CCR5. The exogenous polynucleotide encoding the exogenous gene can be inserted in intron 2 for CLYBL. The exogenous polynucleotide encoding the exogenous gene can be inserted in a 500 bp window in Ch-4:58,976,613 (i.e., SHS231). The exogenous polynucleotide encoding the exogenous gene can be insert in any suitable region of the aforementioned safe harbor or target loci that allows for expression of the exogenous gene, including, for example, an intron, an exon or a coding sequence region in a safe harbor or target locus.

As used herein, a “target” can refer to a gene, a portion of a gene, a portion of the genome, or a protein that is subject to regulatable reduced expression by the methods described herein.

As used herein, a “subject” or an “individual,” which are terms that are used interchangeably, is a mammal. In some embodiments, a “mammal” includes humans, non-human primates, domestic and farm animals, and zoo, sports, or pet animals, such as dogs, horses, rabbits, cattle, pigs, hamsters, gerbils, mice, ferrets, rats, cats, monkeys, etc. In some embodiments, the subject or individual is human. In some embodiments, the subject is a patient that is known or suspected of having a disease, disorder or condition.

As used herein, “therapeutically effective amount” refers to an amount sufficient to provide a therapeutic benefit in the treatment and/or management of a disease, disorder, or condition. In some embodiments, a therapeutically effective amount is an amount sufficient to ameliorate, palliate, stabilize, reverse, slow, attenuate or delay the progression of a disease, disorder, or condition, or of a symptom or side effect of the disease, disorder, or condition. In some embodiments, the therapeutically effective amount is also a clinically effective amount. In other embodiments, the therapeutically effective amount is not a clinically effective amount.

As used herein, the term “treating” and “treatment” includes administering to a subject an effective amount of cells described herein so that the subject has a reduction in at least one symptom of the disease or an improvement in the disease, for example, beneficial or desired clinical results. For purposes of this technology, beneficial or desired clinical results include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable. Treating can refer to prolonging survival as compared to expected survival if not receiving treatment. Thus, one of skill in the art realizes that a treatment may improve the disease condition, but may not be a complete cure for the disease. In some embodiments, one or more symptoms of a disease or disorder are alleviated by at least 5%, at least 10%, at least 20%, at least 30%, at least 40%, or at least 50% upon treatment of the disease.

For purposes of this technology, beneficial or desired clinical results of disease treatment include, but are not limited to, alleviation of one or more symptoms, diminishment of extent of disease, stabilized (i.e., not worsening) state of disease, delay or slowing of disease progression, amelioration or palliation of the disease state, and remission (whether partial or total), whether detectable or undetectable.

A “vector” or “construct” is capable of transferring gene sequences to target cells. Typically, “vector construct,” “expression vector,” and “gene transfer vector,” mean any nucleic acid construct capable of directing the expression of a gene of interest and which can transfer gene sequences to target cells. Thus, the term includes cloning, and expression vehicles, as well as integrating vectors. Methods for the introduction of vectors or constructs into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer.

### VII. Examples

The following examples are included for illustrative purposes only and are not intended to limit the scope of the invention.

Example 1. Immune Evasion of B2M^{indel/indel}, CIITA^{indel/indel}, CD47 tg Stem Cell Derived Beta-Like (SC-b) Cells

This example describes studies to assess the effects of differentiating beta cells from pluripotent stem cells that had been modified with reduced MHC class I and MHC class II expression and increased CD47 expression. In vitro NK cell- and macrophage cell-mediated killing of modified B2M^{indel/indel} CIITA^{indel/indel}, CD47tg hiPSCs, as well as iPSC-derived beta islets (SC-beta) cells differentiated from the modified iPSCs, were monitored over time.

### A. Methods

B2M^{indel/indel}, CIITA^{indel/indel} hiPSCs were generated using standard CRISPR/Cas9 gene editing techniques. A transgene (tg) encoding exogenous CD47 was introduced into the cells by homology directed repair for safe harbor-targeted gene insertion into the AAVS1, CCR5 or CLYBL safe harbor locus. The modified hiPSCs were differentiated to generate iPSC-derived beta islet (SC-beta) cells according to established protocols for beta islet cell differentiation essentially as described Hogrebe et al., “Generation of insulin-producing pancreatic beta cells from multiple human stem cell lines,” Nat. Protoc., 2021, doi: 10.1038/s41596-021-00560-y.

Flow cytometry. Surface expression of MHC class I, MHC class II, and CD47 on modified hiPSCs or SC-beta cells were assessed by flow cytometry using antibody-specific reagents. Isotype antibodies were used as a control.

NK cell and macrophage killing assay. NK cell killing assays and macrophage killing assays were performed on the XCelligence SP platform and MP platform (ACEA Biosciences) to provide for label-free monitoring of cell proliferation and viability of cells. B2M^{indel/indel}, CIITA^{indel/indel}, CD47tg hiPSCs or SC-b cells differentiated therefrom were plated into 96-well collagen coated E-plates. The XCelligence software was used to measure Cell Index (CI) as a measure of adhesion and hence cell killing (a decrease in cell index indicates an increase in killing of the cells). After the CI value reached 0.7, human primary NK cells or human macrophages differentiated from peripheral blood mononuclear cells with macrophage colony stimulating factor (M-CSF) were added at an effector to target (E: T) ratio of 1:1.

### B. Results

B2M^{indel/indel} CIITA^{indel/indel}, CD47tg hiPSCs do not express HLA-I or HLA-II, and have increased CD47 expression and evade NK cell and macrophage killing. Flow cytometry analysis of the modified B2M^{indel/indel}, CIITA^{indel/indel}, CD47tg iPSCs showed that the cells were negative for HLA-I and HLA-II, and had increased expression of CD47 (73-fold, 89-fold or 79-fold for AAVS1, CCR5 and CLYBL safe harbor sites, respectively, over isotype control). In contrast, wild-type iPSCs had high level HLA-I expression and low expression of CD47 (2.3-fold over isotype control). Further, the modified B2M^{indel/indel}, CIITA^{indel/indel}, CD47tg iPSCs did not exhibit NK-mediated cell killing or macrophage-mediated cell killing, with similar protection from innate immune cell killing observed for cells modified in each of the three safe harbor sites. As a positive control, iPSCs with B2M^{indel/indel}, CIITA^{indel/indel} only were rapidly killed by NK cells or macrophages. These results demonstrate that CD47 expression is protecting the cells from killing.

Modified SC-beta cells differentiated from B2M^{indel/indel}, CIITA^{indel/indel}, CD47tg hiPSCs do not express HLA-I or HLA-II, and have increased CD47 expression and evade NK cell and macrophage killing. Flow cytometry analysis of the modified SC-beta cells following differentiation showed that the cells remained negative for HLA-I and HLA-II, and retained increased expression of CD47 (75-fold, 77-fold or 72-fold in cells when differentiated from iPSCs with CD47 modified into AAVS1, CCR5 or CLYBL safe harbor sites, respectively, over isotype control). In contrast, wild-type SC-beta islets differentiated from wild-type hiPSCs had high level HLA-I expression and low expression of CD47 (2.6-fold over isotype control). These results demonstrate that CD47 remains high after iPSC differentiation into beta islet cells for all 3 safe harbor sites.

Modified SC-beta cells derived from modified B2M^{indel/indel}, CIITA^{indel/indel}, CD47tg iPSCs did not exhibit NK-mediated cell killing or macrophage-mediated cell killing, with similar protection from innate immune cell killing observed when differentiated from iPSCs with exogenous CD47 modified into any of the 3 safe harbor sites. These results demonstrate that CD47 expression is maintained on differentiated beta islet cells and protects the cells from killing.

Example 2 In Vitro Characterization of Modified iPSC-Derived Beta Cells Differentiated from B2M^{indel/indel}, CIITA^{indel/indel}, CD47tg hiPSCs

The modified iPSC-derived beta islets (SC-beta) cells generated as described in Example 1 were monitored for insulin secretion.

### A. Methods

Insulin secretion. A standard glucose-stimulated insulin secretion (GSIS) assay was used to measure in vitro insulin secretion. A U-PLEX® Meso Scale Discovery (MSD) assay was used to detect insulin secretion. Briefly, 100,000 cells were used in 2 mL of media containing low glucose (5 mM), and total insulin secretion over 24 h was measured.

Generation of human primary beta islet cells. Primary beta islet cells were isolated from a human donor using a standard technique. Such techniques are known in the art, including as described in J. Kerr-Conte et al., Transplantation, 89, 2010.

### B. Results

Modified SC-beta cells differentiated from B2M^{indel/indel}, CIITA^{indel/indel}, CD47tg hiPSCs retain insulin secretion capabilities. In this experiment, the level of insulin secretion by modified SC-beta cells derived from B2M^{indel/indel}, CIITA^{indel/indel}, CD47tg iPSCs was similar to that of SC-beta cells derived from wild-type hiPSCs. The SC-beta cells derived from both B2M^{indel/indel}, CIITA^{indel/indel}, CD47tg hiPSCs or wild-type hiPSCs exhibited insulin about 25% of the level of insulin secretion by primary human islets. These results indicate that the hypoimmune edits did not impair differentiation or function and demonstrate that the modified SC-beta cells function in vitro.

Example 3. Survival and Function of Modified iPSC-Derived Beta Cells Differentiated from B2M^{indel/indel}, CIITA^{indel/indel} CD47tg hiPSCs in a Transplant Study

The modified SC-beta cells derived from B2M^{indel/indel}, CIITA^{indel/indel}, CD47tg hiPSCs, generated as described in Example 1, were transplanted into humanized NSG diabetes disease mouse model recipients. For comparison, wild-type beta and B2M^{indel/indel}, CIITA^{indel/indel} beta cells were differentiated from wild-type and B2M^{indel/indel}, CIITA^{indel/indel} hiPSCs, respectively. Survival and function of the transplanted modified SC-beta cells compared to transplanted wild type or B2M^{indel/indel} CIITA^{indel/indel} SC-beta cells were monitored over time.

### A. Methods

Diabetes mouse model and transplant study design and administration. Humanized NSG mice were injected with low dose streptozotocin (STZ) (60 mg/kg i.p.) daily for 5 days (day −5 to day 0). STZ was dissolved in citrate buffer (10 mg/ml stock solution) and diluted to an injection volume of 150 μL for intraperitoneal (i.p.) injection. Phosphate buffered saline (PBS) (1 mL) i.p. injections were administered to the mice the next morning to keep the kidneys healthy.

Islet clusters of about 1400-1500 cells per cluster were transplanted by i.m. injection into mice. Day 0 (d0) was defined as the day of transplantation. Mice were monitored for bioluminescence (BLI) as an indicator of beta islet cell survival and for glucose levels to monitor diabetes

Blood glucose measurements. Blood glucose measurements were taken 4 hours after food withdrawal according to standard protocols. Glucose was measured after 4 hours of fasting prior to islet implantation and various days post-implant.

Cell survival. Cell survival was measured by bioluminescence imaging (BLI).

### B. Results

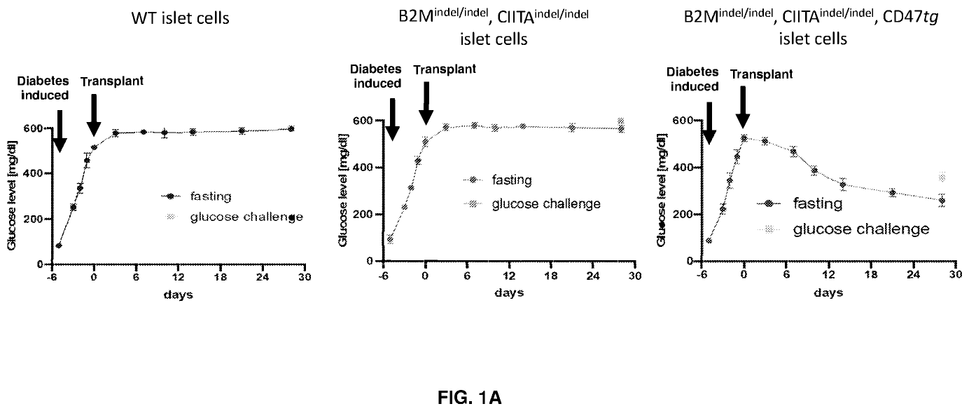

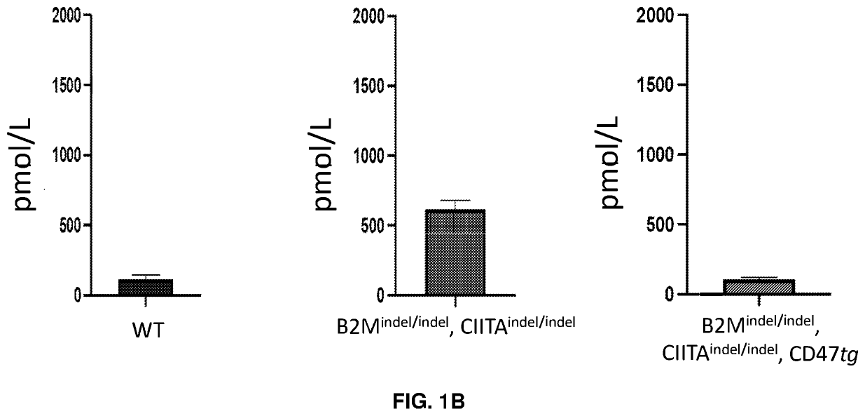

Modified SC-beta cells derived from B2M^{indel/indel}, CIITA^{indel/indel}, CD47tg hiPSCs survive after allogeneic transplant. Quantification of BLI imaging results of modified SC-beta showed detection of cells after transplant that survived up to day 29, when the animals were sacrificed. The number of photons detected from transplanted modified SC-beta cells increased over the course of the study post-transplant, indicating survival and growth of the transplanted cells. In contrast, for wild-type SC-beta cells, bioluminescence was initially observed at the i.m. injection site for all groups following administration of the cells; however, the number of photons detected for transplanted wild-type SC-beta cells rapidly declined over the first 5 days after transplant and falling below the limit of detection by day 10, indicating death of the wild-type SC-beta cells, likely due to an immune response. The number of photons detected similarly declined rapidly for B2M^{indel/indel}, CIITA^{indel/indel} SC-beta cells. Additionally, in animals transplanted with wild-type or B2M^{indel/indel}, CIITA^{indel/indel} SC-beta cells glucose levels continued to rise by about 20%, whereas glucose levels decreased over the course of the study in animals transplanted with the modified B2M^{indel/indel}, CIITA^{indel/indel}, CD47tg SC-beta cells, as shown in FIG. 1A.

The mice were subjected to a glucose challenge on day 29 one hour prior to sacrifice, and the sera of sacrificed mice were collected. The serum levels of human c-peptide were assayed by ELISA (Mercodia). Mice transplanted with modified B2M^{indel/indel}, CIITA^{indel/indel}, CD47tg SC-beta cells had an average human c-peptide level of about 600 pmol/L, whereas mice transplanted with the wild-type or B2M^{indel/indel}, CIITA^{indel/indel} SC-beta cells had human c-peptide levels below the lower limit of quantification (100 pmol/L), as shown in FIG. 1B.

These data indicate that modified SC-beta cells having reduced expression of one or more MHC class I and/or MHC class II molecules and increased expression of one or more tolerogenic factors (e.g., CD47) can survive and modulate glucose in vivo.

Example 4. Survival and Function of Modified iPSC-Derived Beta Cells Differentiated from B2M^{indel/indel}, CIITA^{indel/indel}, or B2M^{indel/indel}, CIITA^{indel/indel}, CD47tg hiPSCs in a Transplant Study

Modified SC-beta cells derived from B2M^{indel/indel}, CIITA^{indel/indel} or B2M^{indel/indel}, CIITA^{indel/indel}, CD47tg hiPSCs, generated as described in Example 1, were transplanted into humanized NSG diabetes disease mouse model recipients. For comparison, wild-type beta cells were differentiated from wild-type hiPSCs. Survival and function of the transplanted modified SC-beta cells compared to transplanted wild type SC-beta cells were monitored over time.

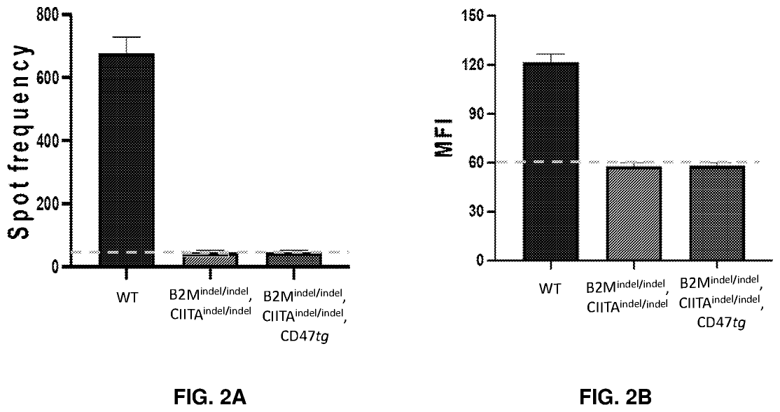

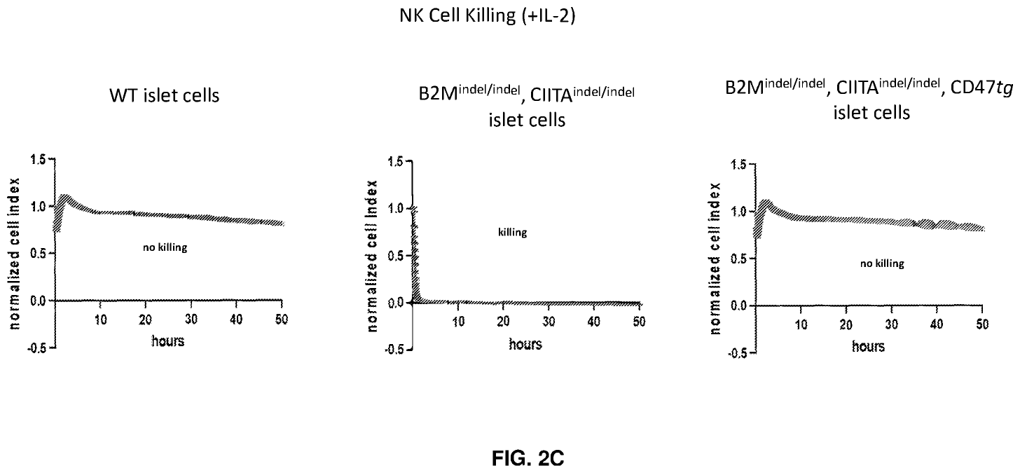

T cell activation. T cell activation in humanized mice administered wild-type, B2M^{indel/indel}, CIITA^{indel/indel}, or B2M^{indel/indel}, CIITA^{indel/indel}, CD47tg human SC-beta cells was measured by Elispot assays. For Elispot assays, recipient splenocytes were isolated from the mice at 6 days post-injection. Donor cells (wild-type or B2M^{indel/indel}, CIITA^{indel/indel} or B2M^{indel/indel}, CIITA^{indel/indel}, CD47tg cells) were mitomycin-treated (50 μg/mL for 30 minutes, Sigma) and used as stimulator cells. 1×10⁵ stimulator cells were incubated with 5×10⁵ recipient responder splenocytes for 24 hours and IFN-γ spot frequencies (for TH1 T cell response) were enumerated using an Elispot plate reader. T cell activation was observed in mice administered wild-type but not B2M^{indel/indel}, CIITA^{indel/indel} or B2M^{indel/indel} CIITA^{indel/indel}, CD47tg human SC-beta cells (FIG. 2A). These results are indicative of systemic TH1 activation and acute cellular immune response after injection of wild-type cells but not cells lacking MHC-I and -II.

Antibody production. Production of donor-specific antibodies by the animals following injection with wild-type, B2M^{indel/indel}, CIITA^{indel/indel}, and B2M^{indel/indel}, CIITA^{indel/indel}, CD47tg human SC-beta cells was also assayed. Sera from recipient mice were de-complemented by heating to 56° C. for 30 minutes. Equal amounts of sera and wild-type, B2M^{indel/indel}, CIITA^{indel/indel}, or B2M^{indel/indel}, CIITA^{indel/indel}, CD47tg cell suspensions (5×10⁶ cells/mL) were incubated for 45 minutes at 4° C. Cells were labelled with FITC-conjugated goat anti-IgM (BD Bioscience) and analyzed by flow cytometry (BD Bioscience). An increase in donor-specific reactivity above pre-injection levels was observed in mice administered wild-type but not B2M^{indel/indel}, CIITA^{indel/indel} or B2M^{indel/indel}, CIITA^{indel/indel}, CD47tg human SC-beta cells (FIG. 2B).

NK cell killing. Systemic innate immunity by natural killer (NK) cells was also assayed using IL-2 stimulated human NK cells as effector and wild-type, B2M^{indel/indel}, CIITA^{indel/indel}, or B2M^{indel/indel}, CIITA^{indel/indel}, CD47tg human SC-beta cells as target cells. NK cell killing assays were performed on the XCELLIGENCE MP platform (ACEA BioSciences). 96-well E-plates (ACEA BioSciences) were coated with collagen (Sigma-Aldrich) and 4×10⁵ wild-type, B2M^{indel/indel}, CIITA^{indel/indel}, or B2M^{indel/indel}, CIITA^{indel/indel}, CD47tg human SC-beta cells were plated in 100 μl cell specific media. After the Cell Index value reached 0.7, human IL2 stimulated NK cells were added with an E:T ratio of 1:1 with or without 1 ng/ml human IL-2. B2M^{indel/indel}, CIITA^{indel/indel} cells were rapidly killed by NK cells, whereas no killing was observed by stimulated or unstimulated NK cells on wild-type or B2M^{indel/indel}, CIITA^{indel/indel}, CD47tg cells (FIG. 2C). This result indicates that CD47 expression on the B2M^{indel/indel}, CIITA^{indel/indel}, CD47tg cells was effective to protect from NK cells in the absence of HLA class I and HLA class II molecules.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

SEQUENCES

| # | SEQUENCE | ANNOTATION |
|---|---|---|
| 1 | QLLFNKTKSVEFTFCNDTVVIPCFVTNMEAQNTTEVYVKWKFKG | Human CD47; aa |
| | RDIYTFDGALNKSTVPTDFSSAKIEVSQLLKGDASLKMDKSDAVS | 19-323 |
| | HTGNYTCEVTELTREGETIIELKYRVVSWFSPNENILIVIFPIFAILL | |
| | FWGQFGIKTLKYRSGGMDEKTIALLVAGLVITVIVIVGAILFVPGE | |
| | YSLKNATGLGLIVTSTGILILLHYYVFSTAIGLTSFVIAILVIQVIAY | |
| | ILAVVGLSLCIAACIPMHGPLLISGLSILALAQLLGLVYMKFVASN | |
| | QKTIQPPRKAVEEPLNAFKESKGMMNDE | |
| 2 | MWPLVAALLLGSACCGSAQLLFNKTKSVEFTFCNDTVVIPCFVT | Human CD47; aa |
| | NMEAQNTTEVYVKWKFKGRDIYTFDGALNKSTVPTDFSSAKIEV | 1-323 |
| | SQLLKGDASLKMDKSDAVSHTGNYTCEVTELTREGETIIELKYRV | |
| | VSWFSPNENILIVIFPIFAILLFWGQFGIKTLKYRSGGMDEKTIALL | |
| | VAGLVITVIVIVGAILFVPGEYSLKNATGLGLIVTSTGILILLHYYV | |
| | FSTAIGLTSFVIAILVIQVIAYILAVVGLSLCIAACIPMHGPLLISGLS | |
| | ILALAQLLGLVYMKFVASNQKTIQPPRKAVEEPLNAFKESKGMM | |
| | NDE | |
| 3 | CEEPPTFEAMELIGKPKPYYEIGERVDYKCKKGYFYIPPLATHTIC | Human CD46, aa |
| | DRNHTWLPVSDDACYRETCPYIRDPLNGQAVPANGTYEFGYQM | 35-392 |
| | HFICNEGYYLIGEEILYCELKGSVAIWSGKPPICEKVLCTPPPKIKN | |
| | GKHTFSEVEVFEYLDAVTYSCDPAPGPDPFSLIGESTIYCGDNSV | |
| | WSRAAPECKVVKCRFPVVENGKQISGFGKKFYYKATVMFECDK | |
| | GFYLDGSDTIVCDSNSTWDPPVPKCLKVLPPSSTKPPALSHSVSTS | |
| | STTKSPASSASGPRPTYKPPVSNYPGYPKPEEGILDSLDVWVIAVI | |
| | VIAIVVGVAVICVVPYRYLQRRKKKGTYLTDETHREVKFTSL | |
| 4 | MEPPGRRECPFPSWRFPGLLLAAMVLLLYSFSDACEEPPTFEAME | Human CD46; aa |
| | LIGKPKPYYEIGERVDYKCKKGYFYIPPLATHTICDRNHTWLPVS | 1-392 |
| | DDACYRETCPYIRDPLNGQAVPANGTYEFGYQMHFICNEGYYLI | |
| | GEEILYCELKGSVAIWSGKPPICEKVLCTPPPKIKNGKHTFSEVEV | |
| | FEYLDAVTYSCDPAPGPDPFSLIGESTIYCGDNSVWSRAAPECKV | |
| | VKCRFPVVENGKQISGFGKKFYYKATVMFECDKGFYLDGSDTIV | |
| | CDSNSTWDPPVPKCLKVLPPSSTKPPALSHSVSTSSTTKSPASSAS | |
| | GPRPTYKPPVSNYPGYPKPEEGILDSLDVWVIAVIVIAIVVGVAVI | |
| | CVVPYRYLQRRKKKGTYLTDETHREVKFTSL | |
| 5 | LQCYNCPNPTADCKTAVNCSSDFDACLITKAGLQVYNKCWKFE | Human CD59; aa |
| | HCNFNDVTTRLRENELTYYCCKKDLCNFNEQLENGGTSLSEKTV | 26-128 |
| | LLLVTPFLAAAWSLHP | |
| 6 | MGIQGGSVLFGLLLVLAVFCHSGHSLQCYNCPNPTADCKTAVNC | Human CD59; aa |
| | SSDFDACLITKAGLQVYNKCWKFEHCNFNDVTTRLRENELTYYC | 1-128 |
| | CKKDLCNFNEQLENGGTSLSEKTVLLLVTPFLAAAWSLHP | |
| 7 | LQCYNCPNPTADCKTAVNCSSDFDACLITKAGLQVYNKCWKFE | Mature Human |
| | HCNFNDVTTRLRENELTYYCCKKDLCNFNEQLEN | CD59; aa 26-102 |
| 8 | DCGLPPDVPNAQPALEGRTSFPEDTVITYKCEESFVKIPGEKDSVI | Human CD55; aa |
| | CLKGSQWSDIEEFCNRSCEVPTRLNSASLKQPYITQNYFPVGTVV | 35-381 |
| | EYECRPGYRREPSLSPKLTCLQNLKWSTAVEFCKKKSCPNPGEIR | |
| | NGQIDVPGGILFGATISFSCNTGYKLFGSTSSFCLISGSSVQWSDPL | |
| | PECREIYCPAPPQIDNGIIQGERDHYGYRQSVTYACNKGFTMIGE | |
| | HSIYCTVNNDEGEWSGPPPECRGKSLTSKVPPTVQKPTTVNVPTT | |
| | EVSPTSQKTTTKTTTPNAQATRSTPVSRTTKHFHETTPNKGSGTTS | |
| | GTTRLLSGHTCFTLTGLLGTLVTMGLLT | |
| 9 | MTVARPSVPAALPLLGELPRLLLLVLLCLPAVWGDCGLPPDVPN | Human CD55; aa |
| | AQPALEGRTSFPEDTVITYKCEESFVKIPGEKDSVICLKGSQWSDI | 1-381 |
| | EEFCNRSCEVPTRLNSASLKQPYITQNYFPVGTVVEYECRPGYRR | |
| | EPSLSPKLTCLQNLKWSTAVEFCKKKSCPNPGEIRNGQIDVPGGIL | |
| | FGATISFSCNTGYKLFGSTSSFCLISGSSVQWSDPLPECREIYCPAP | |
| | PQIDNGIIQGERDHYGYRQSVTYACNKGFTMIGEHSIYCTVNNDE | |
| | GEWSGPPPECRGKSLTSKVPPTVQKPTTVNVPTTEVSPTSQKTTT | |
| | KTTTPNAQATRSTPVSRTTKHFHETTPNKGSGTTSGTTRLLSGHT | |
| | CFTLTGLLGTLVTMGLLT | |
| 10 | DCGLPPDVPNAQPALEGRTSFPEDTVITYKCEESFVKIPGEKDSVI | Mature Human |
| | CLKGSQWSDIEEFCNRSCEVPTRLNSASLKQPYITQNYFPVGTVV | CD55; aa 35-353 |
| | EYECRPGYRREPSLSPKLTCLQNLKWSTAVEFCKKKSCPNPGEIR | |
| | NGQIDVPGGILFGATISFSCNTGYKLFGSTSSFCLISGSSVQWSDPL | |
| | PECREIYCPAPPQIDNGIIQGERDHYGYRQSVTYACNKGFTMIGE | |
| | HSIYCTVNNDEGEWSGPPPECRGKSLTSKVPPTVQKPTTVNVPTT | |
| | EVSPTSQKTTTKTTTPNAQATRSTPVSRTTKHFHETTPNKGSGTTS | |
| 11 | EGRGSLLTCGDVEENPGP | T2A |
| 12 | LEGGGEGRGSLLTCGDVEENPGPR | T2A |
| 13 | GSGATNFSLLKQAGDVEENPGP | P2A |
| 14 | ATNFSLLKQAGDVEENPGP | P2A |
| 15 | QCTNYALLKLAGDVESNPGP | E2A |
| 16 | VKQTLNFDLLKLAGDVESNPGP | F2A |
| 17 | GSGEGRGSLLTCGDVEENPGP | T2A |
| 18 | AAGSGEGRGSLLTCGDVEENPGP | T2A |
| 19 | GUUUUAGAGCUA | Exemplary |
| | | crRNA repeat |
| 20 | UAGCAAGUUAAAAUAAGGCUAGUCCGUUAUCAACUUGAAAA | Exemplary |
| | AGUGGCACCGAGUCGGUGCUUU | tracrRNA |
| 21 | GAAA | Tetraloop |
| | | sequence |

What is claimed:

1. A method of generating a modified stem cell derived beta cell (SC-beta cell), the method comprising:
(A) providing a modified pluripotent stem cell (PSC) comprising modifications that:
(a) inactivate or disrupt one or more alleles of: (i) one or more major histocompatibility complex (MHC) class I molecules or one or more molecules that regulate expression of the one or more MHC class I molecules, and/or (ii) one or more MHC class II molecules or one or more molecules that regulate expression of the one or more MHC class II molecules; and
(b) increase expression of one or more tolerogenic factors in the modified PSC, relative to a control or wild-type PSC; and
(B) culturing the modified PSC under conditions sufficient for differentiation of the modified PSC into the modified SC-beta cell.

2. A method of generating a modified stem cell derived beta cell (SC-beta cell) the method comprising:
(A) generating a modified pluripotent stem cell (PSC) comprising:
(a) introducing, into a PSC, one or more modifications that inactivate or disrupt one or more alleles of: (i) one or more major histocompatibility complex (MHC) class I molecules or one or more molecules that regulate expression of the one or more MHC class I molecules, and/or (ii) one or more MHC class II molecules or one or more molecules that regulate expression of the one or more MHC class II molecules; and
(b) increasing expression of one or more tolerogenic factors in the PSC, relative to a control or wild-type PSC; and
(B) culturing the modified PSC under conditions sufficient for differentiation of the modified PSC into a modified SC-beta cell.

3. A method of generating a modified stem cell derived beta cell (SC-beta cell), the method comprising
(A) providing a modified pluripotent stem cell (PSC) that comprises at least one modification selected from the group consisting of:
(a) modifications that inactivate or disrupt one or more alleles of: (i) one or more major histocompatibility complex (MHC) class I molecules or one or more molecules that regulate expression of the one or more MHC class I molecules, and/or (ii) one or more MHC class II molecules or one or more molecules that regulate expression of the one or more MHC class II molecules; and
(b) modifications that increase expression of one or more tolerogenic factors in the modified PSC, relative to a control or wild-type cell of the same cell type that does not comprise the modification;
(B) culturing the modified PSC under conditions sufficient for differentiation of the modified PSC into a modified SC-beta cell; and
(C) introducing one or more additional modifications into the modified SC-beta cell, wherein the one or more additional modifications comprise at least one or more other modifications of (a), (b), or (a) and (b) not present in the modified PSC.

4. A method of generating a modified stem cell derived beta cell (SC-beta cell), the method comprising
(A) culturing a pluripotent stem cell (PSC) under conditions sufficient for differentiation of the PSC into a SC-beta cell; and
(B) generating a modified SC-beta cells comprising:
(a) introducing, into the SC-beta cell, one or more modifications that inactivate or disrupt one or more alleles of: (i) one or more major histocompatibility complex (MHC) class I molecules or one or more molecules that regulate expression of the one or more MHC class I molecules, and/or (ii) one or more MHC class II molecules or one or more molecules that regulate expression of the one or more MHC class II molecules; and
(b) increasing expression of one or more tolerogenic factors in the SC-beta cell, relative to a control or wild-type SC-beta cell.

5. The method of any of claims 1-4, wherein the modifications in (a) reduce expression of the one or more MHC class I molecules and/or the one or more MHC class II molecules in the modified cell, relative to the control or wild-type cell of the same cell type.

6. The method of any of claims 1-5, wherein the control or wild-type cell is a cell of the same cell type that does not comprise the modifications.

7. The method of any of claims 1-3 and 5-6, wherein expression of the one or more MHC class I molecules and the one or more MHC class II molecules is reduced in the modified PSC relative to the control or wild-type PSC.

8. The method of any of claims 1-7, wherein expression of the one or more MHC class I molecules and the one or more MHC class II molecules is reduced in the modified SC-beta cell relative to the control or wild-type SC-beta cell.

9. The method of any of claims 1-8, wherein the one or more modifications in (a) reduce a function of the one or more MHC class I molecules, optionally wherein the function is antigen presentation.

10. The method of any of claims 1-9, wherein the one or more MHC class I molecules is one or more human leukocyte antigen (HLA) class I molecules.

11. The method of any of claims 1-10, wherein the one or more MHC HLA class I molecules is selected from the group consisting of HLA-A, HLA-B, and HLA-C.

12. The method of any of claims 1-11, wherein the one or more molecules that regulate expression of the one or more MHC class I molecules is/are selected from the group consisting of B2M, NLRC5 and TAP1.

13. The method of any of claims 1-12, wherein the one or more molecules that regulate expression of the one or more MHC class I molecules regulate cell surface protein expression of the one or more MHC class I molecules.

14. The method of any of claims 1-13, wherein the one or more modifications in (a) reduce cell surface protein expression of the one or more MHC class I molecules.

15. The method of any of claims 1-14, wherein the one or more modifications in (a) reduce cell surface trafficking of the one or more MHC class I molecules.

16. The method of claim 14 or claim 15, wherein the one or more molecules that regulate cell surface protein expression of the one or more MHC class I molecules are B2M.

17. The method of any of claims 1-16, wherein the one or more modifications comprise a modification that regulates cell surface protein expression of the one or more MHC class I molecules and the modification inactivates or disrupts one or more alleles of B2M.

18. The method of any of claims 1-17, wherein cell surface trafficking of the one or more MHC class I molecules is reduced in the modified SC-beta cell relative to the control or wild-type SC-beta cell.

19. The method of any of claims 12-18, wherein the modification that inactivates or disrupts one or more alleles of B2M reduces mRNA expression of the B2M gene.

20. The method of any of claims 12-19, wherein the modification that inactivates or disrupts one or more alleles of B2M reduces protein expression of B2M.

21. The method of any of claims 12-20, wherein the modification that inactivates or disrupts one or more alleles of B2M comprises:
inactivation or disruption of one allele of the B2M gene;
inactivation or disruption of both alleles of the B2M gene; or
inactivation or disruption of all B2M coding alleles in the cell.

22. The method of any of claims 12-21, wherein the inactivation or disruption comprises an indel in the B2M gene.

23. The method of any of claims 12-22, wherein the inactivation or disruption comprises a frameshift mutation or a deletion of a contiguous stretch of genomic DNA of the B2M gene.

24. The method of any of claims 1-23, wherein the one or more modifications in (a) reduce cell surface protein expression of the one or more MHC class II molecules.

25. The method of any of claims 1-24, wherein the one or more modifications in (a) reduce cell surface trafficking of the one or more MHC class II molecules.

26. The method of any of claims 1-25, wherein the one or more modifications in (a) reduce a function of the one or more MHC class II molecules, optionally wherein the function is antigen presentation.

27. The method of any of claims 1-26, wherein the one or more MHC class II molecules is one or more human leukocyte antigen (HLA) class II molecules.

28. The method of any of claims 1-27, wherein the one or more MHC HLA class II molecules is selected from the group consisting of HLA-DP, HLA-DQ, and/or HLA-DR.

29. The method of any of claims 1-28, wherein the one or more molecules that regulate expression of the one or more MHC class II molecules is/are selected from the group consisting of CIITA and CD74.

30. The method of any of claims 1-29, wherein the modification is a modification that regulates expression of the one or more MHC class II molecules, and the modification inactivates or disrupts one or more alleles of CIITA.

31. The method of claim 30, wherein the modification that inactivates or disrupts one or more alleles of CIITA reduces mRNA expression of the CIITA gene.

32. The method of claim 30 or 31, wherein the modification that inactivates or disrupts one or more alleles of CIITA reduces protein expression of CIITA.

33. The method of any of claims 30-32, wherein the modification that inactivates or disrupts one or more alleles of CIITA comprises:
inactivation or disruption of one allele of the CIITA gene;
inactivation or disruption of both alleles of the CIITA gene; or
inactivation or disruption of all CIITA coding alleles in the cell.

34. The method of any of claims 30-33, wherein the inactivation or disruption comprises an indel in the CIITA gene.

35. The method of any of claims 30-34, wherein the inactivation or disruption is a frameshift mutation or a deletion of a contiguous stretch of genomic DNA of the CIITA gene.

36. The method of any of claims 1-3 and 5-35, wherein expression of HLA-A, HLA-B, HLA-C, HLA-DP, HLA-DQ, and HLA-DR are reduced in the modified PSC.

37. The method of any of claims 1-36, wherein expression of HLA-A, HLA-B, HLA-C, HLA-DP, HLA-DQ, and HLA-DR are reduced in the modified SC-beta cell.

38. The method of any of claims 1-37, wherein the one or more tolerogenic factors is selected from the group consisting of CD16, CD24, CD35, CD39, CD46, CD47, CD52, CD55, CD59, CD64, CD200, CCL22, CTLA4-Ig, C1 inhibitor, FASL, IDO1, HLA-C, HLA-E, HLA-E heavy chain, HLA-G, IL-10, IL-35, PD-L1, SERPINB9, CCL21, MFGE8, DUX4, B2M-HLA-E, CD27, IL-39, CD16 Fc Receptor, IL15-RF, H2-M3 (HLA-G), A20/TNFAIP3, CR1, HLA-F, and MANF.

39. The method of any of claims 1-38, wherein at least one of the one or more tolerogenic factors is CD47.

40. The method of any of claims 1-39, wherein the one or more tolerogenic factors is CD47.

41. The method of any of claims 1-39, wherein at least one of the one or more tolerogenic factors is PD-L1.

42. The method of any of claims 1-39 and 41, wherein at least one of the one or more tolerogenic factors is HLA-E.

43. The method of any of claims 1-39 and 41-42, wherein at least one of the one or more tolerogenic factors is HLA-G.

44. The method of any of claims 2 and 5-32, wherein increasing expression of the one or more tolerogenic factors comprises introducing a modification that increases expression of the one or more tolerogenic factor in the modified PSC, relative to the control or wild-type PSC.

45. The method of any of claims 1-44, wherein the modification that increases expression of the one or more tolerogenic factors comprises an exogenous polynucleotide encoding the one or more tolerogenic factors.

46. The method of claim 45, wherein the exogenous polynucleotide encoding the one or more tolerogenic factors is integrated into the genome of the modified PSC.

47. The method of claim 45, wherein the exogenous polynucleotide encoding the one or more tolerogenic factors is integrated into the genome of the modified SC-beta cell.

48. The method of claim 46 or claim 47, wherein the exogenous polynucleotide encoding the one or more tolerogenic factors is integrated by non-targeted insertion into the genome of the modified cell, optionally by introduction of the exogenous polynucleotide into the cell using a lentiviral vector.

49. The method of claim 46 or 47, wherein the exogenous polynucleotide encoding the one or more tolerogenic factors is integrated by targeted insertion into a target genomic locus of the cell, optionally wherein the targeted insertion is by nuclease-mediated gene editing with homology-directed repair.

50. A method of generating a modified stem cell derived beta cell (SC-beta cell), the method comprising:
(A) providing a modified pluripotent stem cell (PSC) comprising knock out of the B2M gene, knock out of the CIITA gene, and an exogenous polynucleotide encoding CD47 protein; and
(B) culturing the modified PSC under conditions sufficient for differentiation of the modified PSC into the modified SC-beta cell, optionally wherein the modified PSC has the phenotype B2M^{indel/indel}, CIITA^{indel/indel}; CD47tg.

51. A method of generating a modified stem cell derived beta cell (SC-beta cell), the method comprising:
(A) providing a pluripotent stem cell (PSC);
(B) culturing the PSC under conditions sufficient for differentiation of the PSC into a SC-beta cell; and
(C) generating a modified SC-beta cell from the SC-beta cell by introducing modifications, into the SC-beta cell to knock out the B2M gene and to knock out the CIITA gene, and introducing an exogenous polynucleotide encoding CD47 protein.

52. A method of generating a modified stem cell derived beta cell (SC-beta cell), the method comprising:
(A) providing a modified pluripotent stem cell (PSC) comprising one or more modifications selected from the group consisting of: knock out of the B2M gene, knock out of the CIITA gene, and an exogenous polynucleotide encoding CD47 protein;
(B) culturing the modified PSC under conditions sufficient for differentiation of the modified PSC into a modified SC-beta cell; and
(C) introducing one or more additional modifications into the modified SC-beta cell, wherein the one or more additional modifications comprise at least one or more other modifications selected from the group consisting of knock out of the B2M gene, knock out of the CIITA gene, and an exogenous polynucleotide encoding CD47 protein not present in the modified PSC.

53. The method of claim 50 or claim 52, wherein the modified SC-beta cell has the phenotype B2M^{indel/indel}; CIITA^{indel/indel}; CD47tg.

54. A method of generating a modified stem cell derived beta cell (SC-beta cell), the method comprising:
(A) providing a modified pluripotent stem cell (PSC) comprising knock out of the B2M gene, knock out of the CIITA gene, an exogenous polynucleotide encoding CD47 protein, and an exogenous polynucleotide encoding a safety switch; and
(B) culturing the modified PSC under conditions sufficient for differentiation of the modified PSC into the modified SC-beta cell.

55. The method of claim 54, wherein the modified PSC has the phenotype B2M^{indel/indel}, CIITA^{indel/indel}; CD47tg; safety switch transgene.

56. The method of any of claims 50-55, wherein the exogenous polynucleotide encoding CD47 is integrated by non-targeted insertion into the genome of the modified cell, optionally by introduction of the exogenous polynucleotide into the cell using a lentiviral vector.

57. The method of any of claims 50-56, wherein the exogenous polynucleotide encoding CD47 is integrated by targeted insertion into a target genomic locus of the cell, optionally wherein the targeted insertion is by nuclease-mediated gene editing with homology-directed repair.

58. The method of any of claims 1-57, wherein the modified PSC further comprises a modification to increase expression of an exogenous safety switch.

59. A method of generating a modified stem cell derived beta cell (SC-beta cell), the method comprising:
(A) providing a pluripotent stem cell (PSC);
(B) culturing the PSC under conditions sufficient for differentiation of the PSC into a SC-beta cell; and
(C) generating a modified SC-beta cell from the SC-beta cell by introducing modifications, into the SC-beta cell to knock out the B2M gene and to knock out the CIITA gene, and introducing an exogenous polynucleotide encoding CD47 protein, and an exogenous polynucleotide encoding a safety switch.

60. A method of generating a modified stem cell derived beta cell (SC-beta cell), the method comprising:
(A) providing a modified pluripotent stem cell (PSC) comprising one or more modifications selected from the group consisting of: knock out of the B2M gene, knock out of the CIITA gene, an exogenous polynucleotide encoding CD47 protein, and an exogenous polynucleotide encoding a safety switch;
(B) culturing the modified PSC under conditions sufficient for differentiation of the modified PSC into a modified SC-beta cell; and
(C) introducing one or more additional modifications into the modified SC-beta cell, wherein the one or more additional modifications comprise at least one or more other modifications selected from the group consisting of knock out of the B2M gene, knock out of the CIITA gene, an exogenous polynucleotide encoding CD47, and an exogenous polynucleotide encoding a safety switch not present in the modified PSC.

61. The method of claim 59 or 60, wherein the modified SC-beta cell has the phenotype B2M^{indel/indel}; CIITA^{indel/indel}; CD47tg; safety switch transgene.

62. The method of any of claims 50-61, wherein the exogenous polynucleotide encoding CD47 is integrated by non-targeted insertion into the genome of the modified SC-beta cell, optionally by introduction of the exogenous polynucleotide into the cell using a lentiviral vector.

63. The method of any of claims 50-61, wherein the exogenous polynucleotide encoding CD47 is integrated by targeted insertion into a target genomic locus of the cell, optionally wherein the targeted insertion is by nuclease-mediated gene editing with homology-directed repair.

64. The method of any of claim 1-53, wherein the modified SC-beta cell further comprises a modification to increase expression of an exogenous safety switch.

65. The method of claim 53, wherein the safety switch is a system wherein upon activation, cells downregulate expression of the one or more tolerogenic factors and/or upregulate expression of one or more immune signaling molecules thereby marking the cell for elimination by the host immune system.

66. The method of claim 65, wherein the one or more tolerogenic factors are selected from the group consisting of CD16, CD24, CD35, CD39, CD46, CD47, CD52, CD55, CD59, CD64, CD200, CCL22, CTLA4-Ig, C1 inhibitor, FASL, IDO1, HLA-C, HLA-E, HLA-E heavy chain, HLA-G, IL-10, IL-35, PD-L1, SERPINB9, CCL21, MFGE8, DUX4, B2M-HLA-E, CD27, IL-39, CD16 Fc Receptor, IL15-RF, H2-M3 (HLA-G), A20/TNFAIP3, CR1, HLA-F, and MANF.

67. The method of claim 65 or claim 66, wherein the one or more immune signaling molecules are selected from the group consisting of B2M, HLA-A, HLA-B, HLA-C, HLA-D, HLA-E, RFXANK, CIITA, CTLA-4, PD-1, RAET1E/ULBP4, RAET1G/ULBP5, RAET1H/ULBP2, RAET1/ULBP1, RAET1L/ULBP6, RAET1N/ULBP3, and other ligands of NKG2D.

68. The method of claim 64, wherein the safety switch is a suicide gene.

69. The method of claim 68, wherein the suicide gene is selected from the group consisting of cytosine deaminase (CyD), herpesvirus thymidine kinase (HSV-Tk), an inducible caspase 9 (iCaspase9), and rapamycin-activated caspase 9 (rapaCasp9).

70. The method of claim 67 or claim 68, wherein the safety switch and the one or more tolerogenic factors are expressed from a bicistronic cassette integrated into the genome of the modified cell.

71. The method of any of claims 64-70, wherein the safety switch and CD47 are expressed from a bicistronic cassette integrated into the genome of the modified cell.

72. The method of claim 70 or claim 71, wherein the bicistronic cassette is integrated by non-targeted insertion into the genome of the modified cell, optionally by introduction of the exogenous polynucleotide into the cell using a lentiviral vector.

73. The method of claim 70 or claim 71, wherein the bicistronic cassette is integrated by targeted insertion into a target genomic locus of the cell, optionally wherein the targeted insertion is by nuclease-mediated gene editing with homology-directed repair.

74. The method of claim 63 or claim 73, wherein the target genomic locus is a safe harbor locus, a B2M gene locus, a CIITA gene locus, or a CD142 gene locus.

75. The method of claim 74, wherein the safe harbor locus is selected from the group consisting of: a CCR5 gene locus, a CXCR4 gene locus, a PPP1R12C (also known as AAVS1) gene, an albumin gene locus, a SHS231 locus, a CLYBL gene locus, and a ROSA26 gene locus.

76. The method of any of claims 1-75, wherein the modified PSC comprises a modification that inactivates or disrupts one or more alleles of CD142.

77. The method of claim 76, wherein the modification reduces mRNA expression of the CD142 gene.

78. The method of claim 76 or claim 77, wherein the modification reduces protein expression of CD142.

79. The method of any of claims 76-78, wherein the modification that inactivates or disrupts one or more alleles of CD142 comprises:
inactivation or disruption of one allele of the CD142 gene;
inactivation or disruption of both alleles of the CD142 gene;
inactivation or disruption of all CD142 coding alleles in the cell.

80. The method of any of claims 76-79, wherein the inactivation or disruption comprises an indel in the CD142 gene.

81. The method of any of claims 76-80, wherein the inactivation or disruption is a frameshift mutation or a deletion of a contiguous stretch of genomic DNA of the CD142 gene.

82. The method of any of claims 1-81, wherein the modified PSC comprises a modification that increases expression of one or more complement inhibitors selected from the group consisting of CD46, CD59, CD55, and CD35, relative to the control or wild-type PSC.

83. The method of any of claims 1-82, wherein the modified SC-beta cell comprises a modification that increases expression of one or more complement inhibitors selected from the group consisting of CD46, CD59, CD55, and CD35, relative to the control or wild-type SC-beta cell.

84. The method of claim 82 or claim 83, wherein the modification to increase expression of the one or more complement inhibitors comprises at least one exogenous polynucleotide selected from the group consisting of an exogenous polynucleotide encoding CD46, an exogenous polynucleotide encoding CD59, an exogenous polynucleotide encoding CD55, and an exogenous polynucleotide encoding CD35.

85. The method of any of claims 82-84, wherein the one or more complement inhibitors are CD46 and CD59.

86. The method of any of claims 82-84, or claim 59, wherein the one or more complement inhibitors are CD46, CD59 and CD55.

87. The method of any of claims 84-86, wherein the at least one exogenous polynucleotide is integrated by non-targeted insertion into the genome of the modified PSC, optionally by introduction of the exogenous polynucleotide into the cell using a lentiviral vector.

88. The method of any of claims 84-87, wherein the at least one exogenous polynucleotide is integrated by targeted insertion into a target genomic locus of the cell, optionally wherein the targeted insertion is by nuclease-mediated gene editing with homology-directed repair.

89. The method of claim 88, wherein the target genomic locus is a safe harbor locus, a B2M gene locus, a CIITA gene locus, or a CD142 gene locus.

90. The method of claim 89, wherein the safe harbor locus is selected from the group consisting of: a CCR5 gene locus, a CXCR4 gene locus, a PPP1R12C (also known as AAVS1) gene, an albumin gene locus, a SHS231 locus, a CLYBL gene locus, and a ROSA26 gene locus.

91. The method of any of claims 1-90, wherein culturing the PSC under conditions sufficient for differentiation of the PSC into the SC-beta cell comprises one or more of:
(i) contacting the PSC with a TGFbeta/Activin agonist and/or, a glycogen synthase kinase 3 (GSK) inhibitor and/or WNT agonist for an amount of time sufficient to form a definitive endoderm cell;
(ii) contacting a definitive endoderm cell differentiated from the PSC with a FGFR2b agonist for an amount of time sufficient to form a primitive gut tube cell;
(iii) contacting a primitive gut tube cell differentiated from the PSC with a retinoic acid receptor (RAR) agonist, a rho kinase inhibitor, a Smoothened antagonist, a FGFR2b agonist, a protein kinase C activator, and/or a BMP type 1 receptor inhibitor for an amount of time sufficient to form an early pancreas progenitor cell;
(iv) incubating an early pancreas progenitor cell differentiated from the PSC for at least about 3 days and contacting the early pancreas progenitor cell with a rho kinase inhibitor, a TGFbeta-/Activin agonist, a Smoothened antagonist, an FGFR2b agonist, a RAR agonist, a protein kinase C activator, and/or a BMP type 1 receptor inhibitor for an amount of time sufficient to form a pancreatic progenitor cell, wherein the RAR agonist concentration is less than the RAR agonist concentration in step (iii);
(v) contacting a pancreatic progenitor cell differentiated from the PSC with an Alk5 inhibitor/TGFbeta receptor inhibitor, a gamma secretase inhibitor, a Smoothened antagonist, an Erbb1 (EGFR) or Erbb4 agonist, a thyroid hormone, and/or a RAR agonist for an amount of time sufficient to form an endoderm cell, wherein during at least a portion of the contacting in (v) comprises depolymerizing the actin cytoskeleton at a time and for an amount of time sufficient to increase differentiation efficiency; and/or
(vi) incubating an endoderm cell differentiated from the PSC for an amount of time in serum-free media sufficient to form a beta cell.

92. The method of any of claims 1-90, wherein the culturing the PSC under conditions sufficient for differentiation of the PSC into the SC-beta cell comprises:
(i) contacting the PSC with a TGFbeta/Activin agonist and/or, a glycogen synthase kinase 3 (GSK) inhibitor and/or WNT agonist for an amount of time sufficient to form a definitive endoderm cell;
(ii) contacting a definitive endoderm cell differentiated from the PSC with a FGFR2b agonist for an amount of time sufficient to form a primitive gut tube cell;
(iii) contacting a primitive gut tube cell differentiated from the PSC with a retinoic acid receptor (RAR) agonist, a rho kinase inhibitor, a Smoothened antagonist, a FGFR2b agonist, a protein kinase C activator, and/or a BMP type 1 receptor inhibitor for an amount of time sufficient to form an early pancreas progenitor cell;
(iv) incubating an early pancreas progenitor cell differentiated from the PSC for at least about 3 days and contacting the early pancreas progenitor cell with a rho kinase inhibitor, a TGFbeta-/Activin agonist, a Smoothened antagonist, an FGFR2b agonist, a RAR agonist, a protein kinase C activator, and/or a BMP type 1 receptor inhibitor for an amount of time sufficient to form a pancreatic progenitor cell, wherein the RAR agonist concentration is less than the RAR agonist concentration in step (iii);
(v) contacting a pancreatic progenitor cell differentiated from the PSC with an Alk5 inhibitor/TGFbeta receptor inhibitor, a gamma secretase inhibitor, a Smoothened antagonist, an Erbb1 (EGFR) or Erbb4 agonist, a thyroid hormone, and/or a RAR agonist for an amount of time sufficient to form an endoderm cell, wherein during at least a portion of the contacting in (v) comprises depolymerizing the actin cytoskeleton at a time and for an amount of time sufficient to increase differentiation efficiency; and
(vi) incubating an endoderm cell differentiated from the PSC for an amount of time in serum-free media sufficient to form a beta cell.

93. The method of claim 91 or 92, wherein the method comprises aggregating the beta cells formed in step (vi) into clusters.

94. The method of any of claims 91-93, wherein depolymerizing the actin cytoskeleton comprises plating cells on a stiff or soft substrate and/or introducing a cytoskeletal-modulating agent to cells.

95. The method of claim 94, wherein the cytoskeletal-modulating agent comprises latrunculin A, latrunculin B, nocodazole, cytochalasin D, jasplakinolide, blebbistatin, y-27632, y-15, gdc-0994, and/or an integrin modulating agent.

96. The method of claim 94 or claim 95, wherein the cytoskeletal-modulating agent is latrunculin A.

97. The method of any of claims 91-96, wherein depolymerizing the actin cytoskeleton is initiated at the start of the contacting in (v).

98. The method of any of claims 91-97, wherein depolymerizing the actin cytoskeleton comprises adding latrunculin A at the start of the contacting for at least at or about the first 24 hours.

99. The method of any of claims 91-98, wherein resizing the beta cell clusters comprises breaking apart clusters and reaggregating.

100. The method of any of claims 91-99, wherein:
the TGF/Activin agonist is Activin A;
the glycogen synthase kinase 3 (GSK) inhibitor or the WNT agonist is CHIR99021;
the FGFR2b agonist is KGF;
the smoothened antagonist is SANT-1;
the RAR agonist is retinoic acid (RA);
the protein kinase C activator is TPPB;
the BMP type 1 receptor inhibitor is LDN193189;
the rho kinase inhibitor is Y27632;
the Alk5 inhibitor is Alk5i II;
the Erbb4 agonist is betacellulin;
the thyroid hormone is T3; and/or
the gamma secretase inhibitor is XXI.

101. The method of any of claims 91-100, wherein the RAR agonist concentration in step (iv) is at least 5-fold, at least 10-fold, or at least 20-fold less than the RAR agonist concentration in step (iii).

102. The method of any of claims 1-101, wherein the PSC is an embryonic stem cell.

103. The method of any of claims 1-101, wherein the PSC is an induced PSC (iPSC), optionally a patient-derived iPSC.

104. The method of any of claims 1-3 and 5-103, wherein the modified PSC expresses each of the one or more tolerogenic factors at a first level that is greater than at or about 5-fold over a second level expressed by the control or wild-type PSC.

105. The method of claim 104, wherein each of the one or more tolerogenic factors is expressed by the modified PSC at a first level that is greater than at or about 10-fold, greater than at or about 20-fold, greater than at or about 30-fold, greater than at or about 40-fold, greater than at or about 50-fold, greater than at or about 60-fold, or greater than at or about 70-fold over a second level expressed by the control or wild-type PSC.

106. The method of any of claims 1-3 and 5-105, wherein each of the one or more tolerogenic factors is expressed by the modified PSC at greater than at or about 20,000 molecules per cell, optionally, wherein each of the one or more tolerogenic factors is expressed by the modified PSC at greater than at or about 30,000 molecules per cell, greater than at or about 50,000 molecules per cell, greater than at or about 100,000 molecules per cell, greater than at or about 200,000 molecules per cell, greater than at or about 300,000 molecules per cell, greater than at or about 400,000 molecules per cell, greater than at or about 500,000 molecules per cell, or greater than at or about 600,000 molecules per cell.

107. The method of any of claims 1-3 and 5-106, wherein the one or more tolerogenic factors comprises CD47 and the modified PSC expresses CD47 at a first level that is greater than at or about 5-fold over a second level expressed by the control or wild-type PSC, optionally wherein CD47 is expressed at a first level that is greater than at or about 10-fold, greater than at or about 20-fold, greater than at or about 30-fold, greater than at or about 40-fold, greater than at or about 50-fold, greater than at or about 60-fold, or greater than at or about 70-fold over a second level expressed by the control or wild-type PSC.

108. The method of any of claims 1-3 and 5-108, wherein the one or more tolerogenic factors comprises CD47 and CD47 is expressed by the modified PSC at greater than at or about 20,000 molecules per cell, optionally, wherein CD47 is expressed by the modified PSC at greater than at or about 30,000 molecules per cell, greater than at or about 50,000 molecules per cell, greater than at or about 100,000 molecules per cell, greater than at or about 200,000 molecules per cell, greater than at or about 300,000 molecules per cell, greater than at or about 400,000 molecules per cell, greater than at or about 500,000 molecules per cell, or greater than at or about 600,000 molecules per cell.

109. The method of any of claims 1-108, wherein the modifications in (a) reduce expression of one or more MHC class I molecules and/or one or more MHC class II molecules in the modified SC-beta cell, relative to a control or wild-type beta cell; and and wherein the modified SC-beta cell has increased expression of one or more tolerogenic factors in the modified SC-beta cell, relative to the control or wild-type beta cell.

110. The method of claim 109, wherein the control or wild-type beta cell is an unmodified SC-beta cell differentiated from an PSC that does not comprise the modifications.

111. The method of claim 109 or claim 110, wherein expression of one or more MHC class I molecules and one or more MHC class II molecules is reduced in the modified SC-beta cell.

112. The method of any of claims 1-3 and 5-111, wherein the modified SC-beta cell comprises the modifications of the modified PSC.

113. The method of any of claims 1-112, wherein the one or more modifications in (a) reduce cell surface protein expression of the one or more MHC class I molecules, optionally wherein the one or more modifications in (a) reduce cell surface trafficking of the one or more MHC class I molecules.

114. The method of any of claims 1-113, wherein the one or more modifications in (a) reduce a function of the one or more MHC class I molecules, optionally wherein the function is antigen presentation.

115. The method of any of claims 1-114, wherein the one or more modifications comprise a modification that regulates cell surface protein expression of the one or more MHC class I molecules and the modification inactivates or disrupts one or more alleles of B2M.

116. The method of any of claims 1-114, wherein the one or more modifications in (a) reduce cell surface trafficking of the one or more MHC class I molecules.

117. The method of any of claims 115-116, wherein the modification that inactivates or disrupts one or more alleles of B2M reduces mRNA expression of the B2M gene, and/or wherein the modification that inactivates or disrupts one or more alleles of B2M reduces protein expression of B2M.

118. The method of any of claims 115-117, wherein the modification that inactivates or disrupts one or more alleles of B2M comprises:
inactivation or disruption of one allele of the B2M gene;
inactivation or disruption of both alleles of the B2M gene; or
inactivation or disruption of all B2M coding alleles in the cell.

119. The method of any of claims 115-118, wherein the inactivation or disruption comprises an indel in the B2M gene.

120. The method of any of claims 115-119, wherein the inactivation or disruption comprises a frameshift mutation or a deletion of a contiguous stretch of genomic DNA of the B2M gene.

121. The method of any of claims 1-120, wherein the one or more modifications in (a) reduce cell surface protein expression of the one or more MHC class II molecules.

122. The method of any of claims 1-121, wherein the one or more modifications in (a) reduce cell surface trafficking of the one or more MHC class II molecules.

123. The method of any of claims 1-122, wherein the modifications in (a) reduce a function of the one or more MHC class II molecules, optionally wherein the function is antigen presentation.

124. The method of any of claims 1-123, wherein the one or more modifications comprise a modification that regulates expression of the one or more MHC class II molecules and the modification inactivates or disrupts one or more alleles of CIITA.

125. The method of claim 124, wherein the modification that inactivates or disrupts one or more alleles of CIITA reduces mRNA expression of the CIITA gene, or wherein the modification that inactivates or disrupts one or more alleles of one or more molecules that regulate expression of the one or more MHC class II molecules in the modified SC-beta cell reduces protein expression of CIITA.

126. The method of any of claims 124-125, wherein the modification that inactivates or disrupts one or more alleles of CIITA comprises:
inactivation or disruption of one allele of the CIITA gene;
inactivation or disruption of both alleles of the CIITA gene; or
inactivation or disruption of all CIITA coding alleles in the cell.

127. The method of any of claims 124-126, wherein the inactivation or disruption comprises an indel in the CIITA gene.

128. The method of any of claims 124-127, wherein the inactivation or disruption is a frameshift mutation or a deletion of a contiguous stretch of genomic DNA of the CIITA gene.

129. The method of any of claims 1-128, wherein expression of HLA-A, HLA-B, HLA-C, HLA-DP, HLA-DQ, and HLA-DR are reduced in the modified SC-beta cell.

130. The method of any of claims 1-129, wherein the modified SC-beta cell comprises a modification that inactivates or disrupts one or more alleles of CD142.

131. The method of claim 130, wherein the modification reduces mRNA expression of the CD142 gene.

132. The method of claim 130 or claim 131, wherein the modification reduces protein expression of CD142.

133. The method of any of claims 130-132, wherein the modification that inactivates or disrupts one or more alleles of CD142 comprises:
inactivation or disruption of one allele of the CD142 gene;
inactivation or disruption of both alleles of the CD142 gene; or
inactivation or disruption of all CD142 coding alleles in the cell.

134. The method of any of claims 130-133, wherein the inactivation or disruption comprises an indel in the CD142 gene.

135. The method of any of claims 130-134, wherein the inactivation or disruption is a frameshift mutation or a deletion of a contiguous stretch of genomic DNA of the CD142 gene.

136. The method of any of claims 1-135, wherein the inactivation or disruption of the one or more alleles is by one or more gene edits.

137. The method of any of claims 1-136, wherein the cell comprises a genome editing complex.

138. The method of claim 136 or claim 137, wherein the one or more gene edits are made by a genome editing complex.

139. The method of claim 138, wherein the genome editing complex comprises a genome targeting entity and a genome modifying entity.

140. The method of claim 139, wherein the genome targeting entity localizes the genome editing complex to the one or more alleles that are inactivated or disrupted, optionally wherein the genome targeting entity is a nucleic acid-guided targeting entity.

141. The method of claim 139 or claim 140, wherein the genome targeting entity is selected from the group consisting of a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising the gRNA and the Cas nuclease, a homing endonuclease, a zinc finger nuclease (ZF) nucleic acid binding entity, a transcription activator-like effector (TALE) nucleic acid binding entity, a meganuclease, a Cas nuclease, a core Cas protein, a homing endonuclease, an endonuclease-deficient-Cas protein, an enzymatically inactive Cas protein, a CRISPR-associated transposase (CAST), a Type II or Type V Cas protein, or a functional portion thereof.

142. The method of any one of claims 139-141, wherein the genome targeting entity is selected from the group consisting of Cas1, Cas2, Cas3, Cas4, Cas5, Cas6, Cas7, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr1, Cmr2, Cmr3, Cmr4, Cmr5, Cmr6, Csd1, Csd2, Cas5d, Cse1, Cse2, Cse3, Cse4, Cas5e, Csf1, Csm1, Csm2, Csm3, Csm4, Csm5, Csn1, Csn2, Cst1, Cst2, Cas5t, Csh1, Csh2, Cas5h, Csa1, Csa2, Csa3, Csa4, Csa5, Cas5a, Csx10, Csx11, Csy1, Csy2, Csy3, Csy4, Mad7, SpCas9, eSpCas9, SpCas9-HF1, HypaSpCas9, HeFSpCas9, and evoSpCas9 high-fidelity variants of SpCas9, SaCas9, NmeCas9, CjCas9, StCas9, TdCas9, LbCas12a, AsCas12a, AacCas12b, BhCas12b v4, TnpB, dCas (D10A), dCas (H840A), dCas13a, dCas13b, or a functional portion thereof.

143. The method of any of claims 139-141, wherein the genome modifying entity cleaves, deaminates, nicks, polymerizes, interrogates, integrates, cuts, unwinds, breaks, alters, methylates, demethylates, or otherwise destabilizes the target locus.

144. The method of any of claims 139-143, wherein the genome modifying entity comprises a recombinase, integrase, transposase, endonuclease, exonuclease, nickase, helicase, DNA polymerase, RNA polymerase, reverse transcriptase, deaminase, flippase, methylase, demethylase, acetylase, a nucleic acid modifying protein, an RNA modifying protein, a DNA modifying protein, an Argonaute protein, an epigenetic modifying protein, a histone modifying protein, or a functional portion thereof.

145. The method of any of claims 139-144, wherein the genome modifying entity selected from the group consisting of a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising the gRNA and the Cas nuclease, a homing endonuclease, a zinc finger nuclease (ZFN), a transcription activator-like effector nuclease (TALEN), a meganuclease, a Cas nuclease, a core Cas protein, a homing endonuclease, an endonuclease-deficient-Cas protein, an enzymatically inactive Cas protein, a CRISPR-associated transposase (CAST), a Type II or Type V Cas protein, base editing, prime editing, a Programmable Addition via Site-specific Targeting Elements (PASTE), or a functional portion thereof.

146. The method of any of claims 139-145, wherein the genome modifying entity is selected from the group consisting of Cas1, Cas2, Cas3, Cas4, Cas5, Cas6, Cas7, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr1, Cmr2, Cmr3, Cmr4, Cmr5, Cmr6, Csd1, Csd2, Cas5d, Cse1, Cse2, Cse3, Cse4, Cas5e, Csf1, Csm1, Csm2, Csm3, Csm4, Csm5, Csn1, Csn2, Cst1, Cst2, Cas5t, Csh1, Csh2, Cas5h, Csa1, Csa2, Csa3, Csa4, Csa5, Cas5a, Csx10, Csx11, Csy1, Csy2, Csy3, Csy4, Mad7, SpCas9, eSpCas9, SpCas9-HF1, HypaSpCas9, HeFSpCas9, and evoSpCas9 high-fidelity variants of SpCas9, SaCas9, NmeCas9, CjCas9, StCas9, TdCas9, LbCas12a, AsCas12a, AacCas12b, BhCas12b v4, TnpB, FokI, dCas (D10A), dCas (H840A), dCas13a, dCas13b, a base editor, a prime editor (e.g., a target-primed reverse transcription (TPRT) editor), APOBEC1, cytidine deaminase, adenosine deaminase, uracil glycosylase inhibitor (UGI), adenine base editors (ABE), cytosine base editors (CBE), reverse transcriptase, serine integrase, recombinase, transposase, polymerase, adenine-to-thymine or “ATBE” (or thymine-to-adenine or “TABE”) transversion base editor, ten-eleven translocation methylcytosine dioxygenases (TETs), TET1, TET3, TET1CD, histone acetyltransferase p300, histone methyltransferase SMYD3, histone methyltransferase PRDM9, H3K79 methyltransferase DOT1L, transcriptional repressor, or a functional portion thereof.

147. The method of any of claims 139-146, wherein the genome targeting entity and the genome modifying entity are different domains of a single polypeptide.

148. The method of any of claims 139-147, wherein the genome editing entity and genome modifying entity are two different polypeptides that are operably linked together.

149. The method of any of claims 139-147, wherein the genome editing entity and genome modifying entity are two different polypeptides that are not linked together.

150. The method of any of claims 139-147, wherein the genome editing complex comprises a guide nucleic acid having a targeting domain that is complementary to at least one target locus, optionally wherein the guide nucleic acid is a guide RNA (gRNA).

151. The method according to any one of claims 139-150, wherein the one or more modifications are made by the genome editing complex.

152. The method according to claim 151, wherein the one or more modifications made by the genome editing complex are made by a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising the gRNA and the Cas nuclease, a homing endonuclease, a zinc finger nuclease (ZFN), a transcription activator-like effector nuclease (TALEN), a meganuclease, a Cas nuclease, a core Cas protein, a TnpB nuclease, a homing endonuclease, an endonuclease-deficient-Cas protein, an enzymatically inactive Cas protein, a CRISPR-associated transposase (CAST), a Type II or Type V Cas protein, base editing, prime editing, or a Programmable Addition via Site-specific Targeting Elements (PASTE).

153. The method according to claim 151 or claim 152, wherein the one or more modifications made by the genome editing complex are made by Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, a CRISPR-associated transposase, base editing, prime editing, or Programmable Addition via Site-specific Targeting Elements (PASTE).

154. The method of any of claims 151-153, wherein the modifications made by the genome editing complex are made using a guide RNA (gRNA) having a targeting domain that is complementary to at least one target site.

155. The method of any of claims 137-138, wherein the genome editing complex is an RNA-guided nuclease.

156. The method of claim 155, wherein the RNA-guided nuclease comprises a Cas nuclease and a guide RNA (CRISPR-Cas combination).

157. The method of claim 156, wherein the CRISPR-Cas combination is a ribonucleoprotein (RNP) complex comprising the gRNA and the Cas nuclease.

158. The method of claim 156 or claim 157, wherein the Cas nuclease is a Type II or Type V Cas protein.

159. The method of any of claims 156-158, wherein the genome-modifying protein is selected from the group consisting of Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, and a CRISPR-associated transposase, or a homologue of any of the foregoing.

160. The method of any of claims 1-159, wherein the modification that increases expression of the one or more tolerogenic factors in the modified SC-beta cell comprises an exogenous polynucleotide encoding the one or more tolerogenic factors.

161. The method of claim 160, wherein the exogenous polynucleotide encoding the one or more tolerogenic factors is integrated into the genome of the modified SC-beta cell.

162. The method of claim 161, wherein the exogenous polynucleotide is integrated into a non-target locus in the genome of the modified SC-beta cell.

163. The method of claim 161, wherein the exogenous polynucleotide is integrated into a target genomic locus of the modified SC-beta cell.

164. The method of any of claims 1-163, wherein the modified SC-beta cell further comprises a modification for expression of an exogenous safety switch.

165. The method of claim 164, wherein the safety switch is a system wherein upon activation, cells downregulate expression of the one or more tolerogenic factors and/or upregulate expression of one or more immune signaling molecules thereby marking the cell for elimination by the host immune system.

166. The method of claim 165, wherein the one or more tolerogenic factors are selected from the group consisting of CD16, CD24, CD35, CD39, CD46, CD47, CD52, CD55, CD59, CD64, CD200, CCL22, CTLA4-Ig, C1 inhibitor, FASL, IDO1, HLA-C, HLA-E, HLA-E heavy chain, HLA-G, IL-10, IL-35, PD-L1, SERPINB9, CCL21, MFGE8, DUX4, B2M-HLA-E, CD27, IL-39, CD16 Fc Receptor, IL15-RF, H2-M3 (HLA-G), A20/TNFAIP3, CR1, HLA-F, and MANF.

167. The method of claim 165 or claim 166, wherein the one or more immune signaling molecules are selected from the group consisting of B2M, HLA-A, HLA-B, HLA-C, HLA-D, HLA-E, RFXANK, CIITA, CTLA-4, PD-1, RAET1E/ULBP4, RAET1G/ULBP5, RAET1H/ULBP2, RAET1/ULBP1, RAET1L/ULBP6, RAET1N/ULBP3, and other ligands of NKG2D.

168. The method of claim 164, wherein the safety switch is a suicide gene.

169. The method of claim 168, wherein the suicide gene is selected from the group consisting of cytosine deaminase (CyD), herpesvirus thymidine kinase (HSV-Tk), an inducible caspase (iCaspase9), and rapamycin-activated caspase 9 (rapaCasp9).

170. The method of any of claims 164-169, wherein the safety switch and the one or more tolerogenic factors are expressed from a bicistronic cassette integrated into the genome of the modified SC-beta cell.

171. The method of claim 170, wherein the bicistronic cassette is integrated at a non-target locus in the genome of the modified SC-beta cell.

172. The method of claim 170, wherein the bicistronic cassette is integrated into a target genomic locus of the cell.

173. The method of claim 163 or claim 172, wherein the target genomic locus is a safe harbor locus, a B2M gene locus, a CIITA gene locus, or a CD142 gene locus.

174. The method of claim 173, wherein the safe harbor locus is selected from the group consisting of: a CCR5 gene locus, a CXCR4 gene locus, a PPP1R12C (also known as AAVS1) gene, an albumin gene locus, a SHS231 locus, a CLYBL gene locus, and a ROSA26 gene locus.

175. The method of any of claims 1-174, wherein the modified SC-beta cell comprises a modification that increases expression of one or more complement inhibitors selected from the group consisting of CD46, CD59, CD55, and CD35 relative to the control or wild-type beta cell.

176. The method of claim 175, wherein the modification to increase expression of the one or more complement inhibitors in the modified SC-beta cell comprises at least one exogenous polynucleotide encoding the one or more complement inhibitors selected from the group consisting of an exogenous polynucleotide encoding CD46, an exogenous polynucleotide encoding CD59, an exogenous polynucleotide encoding CD55, and an exogenous polynucleotide encoding CD35.

177. The method of claim 175 or claim 176, wherein the one or more complement inhibitors are CD46 and CD59.

178. The method of claim 175 or claim 176, wherein the one or more complement inhibitors are CD46, CD59 and CD55.

179. The method of any of claims 5-178, wherein the reduced expression comprises reduced cell surface expression.

180. The method of any of claims 1-179, wherein the increased expression comprises increased cell surface expression.

181. The method of any of claims 1-3 and 5-180, wherein the level of the reduced expression of (a) and the increased expression of (b) by the modified SC-beta cell is retained or is similar compared to the modified PSC.

182. The method of any of claims 1-181, wherein the modified SC-beta cell expresses the one or more tolerogenic factors at a first level that is greater than at or about 5-fold over a second level expressed by the control or wild-type beta cell, optionally wherein the control or wild-type beta cell is differentiated from a PSC not comprising modifications that inactivate or disrupt one or more alleles of: (i) one or more major histocompatibility complex (MHC) class I molecules or one or more molecules that regulate expression of the one or more MHC class I molecules, and/or (ii) one or more MHC class II molecules or one or more molecules that regulate expression of the one or more MHC class II molecules; and that increase expression of one or more tolerogenic factors.

183. The method of any of claims 1-182, wherein the modified SC-beta cell expresses each of the one or more tolerogenic factors at a first level that is greater than at or about 5-fold over a second level expressed by the control or wild-type beta cell.

184. The method of claim 183, wherein each of the one or more tolerogenic factor is expressed at a first level that is greater than at or about 10-fold, greater than at or about 20-fold, greater than at or about 30-fold, greater than at or about 40-fold, greater than at or about 50-fold, greater than at or about 60-fold, or greater than at or about 70-fold over a second level expressed by the control or wild-type beta cell.

185. The method of any of claims 1-184, wherein each of the one or more tolerogenic factors is expressed by the modified SC-beta cell at greater than at or about 20,000 molecules per cell.

186. The method of claim 185, wherein each of the one or more tolerogenic factors is expressed by the modified SC-beta cell at greater than at or about 30,000 molecules per cell, greater than at or about 50,000 molecules per cell, greater than at or about 100,000 molecules per cell, greater than at or about 200,000 molecules per cell, greater than at or about 300,000 molecules per cell, greater than at or about 400,000 molecules per cell, greater than at or about 500,000 molecules per cell, or greater than at or about 600,000 molecules per cell.

187. The method of any of claims 1-186, wherein the one or more tolerogenic factors comprises CD47 and the modified SC-beta cell expresses CD47 at a first level that is greater than at or about 5-fold over a second level expressed by the control or wild-type beta cell, optionally wherein the control or wild-type beta cell is differentiated from a control or wild-type PSC not comprising modifications that inactivate or disrupt one or more alleles of: (i) one or more major histocompatibility complex (MHC) class I molecules or one or more molecules that regulate expression of the one or more MHC class I molecules, and/or (ii) one or more MHC class II molecules or one or more molecules that regulate expression of the one or more MHC class II molecules; and that increase expression of one or more tolerogenic factors.

188. The method of any of claims 1-187, wherein the one or more tolerogenic factors comprises CD47 and the modified SC-beta cell expresses CD47 at a first level that is greater than at or about 5-fold over a second level expressed by the control or wild-type beta cell.

189. The method of claim 188, wherein CD47 is expressed at a first level that is greater than at or about 10-fold, greater than at or about 20-fold, greater than at or about 30-fold, greater than at or about 40-fold, greater than at or about 50-fold, greater than at or about 60-fold, or greater than at or about 70-fold over a second level expressed by the control or wild-type beta cell.

190. The method of any of claims 1-189, wherein the one or more tolerogenic factors comprises CD47 and CD47 is expressed by the modified SC-beta cell at greater than at or about 20,000 molecules per cell.

191. The method of claim 190, wherein CD47 is expressed by the modified SC-beta cell at greater than at or about 30,000 molecules per cell, greater than at or about 50,000 molecules per cell, greater than at or about 100,000 molecules per cell, greater than at or about 200,000 molecules per cell, greater than at or about 300,000 molecules per cell, greater than at or about 400,000 molecules per cell, greater than at or about 500,000 molecules per cell, or greater than at or about 600,000 molecules per cell.

192. The method of any of claims 1-191, wherein the modified SC-beta cell expresses at least one beta cell marker, optionally wherein the at least one beta cell marker is selected from the group consisting of INS, CHGA, NKX2-2, PDX1, NKX6-1, MAFB, GCK and GLUT1.

193. The method of any of claims 1-192, wherein the modified SC-beta cell exhibits one or more functions of a wild-type or control beta cell, optionally wherein the one or more functions is selected from the group consisting of in vitro glucose-stimulated insulin secretion (GSIS), glucose metabolism, maintaining fasting blood glucose levels, secreting insulin in response to glucose injections in vivo, and clearing glucose after a glucose injection in vivo.

194. The method of any of claims 1-193, wherein the modified SC-beta cell is capable of glucose-stimulated insulin secretion (GSIS), optionally wherein the insulin secretion is in a perfusion GSIS assay.

195. The method of claim 194, wherein the GSIS is dynamic GSIS comprising first and second phase dynamic insulin secretion.

196. The method of claim 194, wherein the GSIS is static GSIS, optionally wherein the static incubation index is greater than at or about 1, greater than at or about 2, greater than at or about 5, greater than at or about 10 or greater than at or about 20.

197. The method of any of claims 1-196, wherein the level of insulin secretion by the modified SC-beta cells is at least 20% of that observed for primary beta islets, optionally cadaveric islets.

198. The method of claim 197, wherein the level of insulin secretion by the modified SC-beta cells is at least 25%, at least 30%, at least 40%, at least 50%, at least 60%, at least 70% of that observed for primary beta islets, optionally cadaveric islets.

199. The method of any of claims 1-198, wherein the total insulin content of the modified SC-beta cell is greater than at or about 500 μIU Insulin per 5000 cells, greater than at or about 1000 μIU Insulin per 5000 cells, greater than at or about 2000 μIU Insulin per 5000 cells, greater than at or about 3000 μIU Insulin per 5000 cells or greater than at or about 4000 μIU Insulin per 5000 cells.

200. The method of any of claims 1-199, wherein the proinsulin to insulin ratio of the modified SC-beta cell is between at or about 0.02 and at or about 0.1, optionally at or about 0.03, 0.04, 0.05, 0.06, 0.07, 0.08, 0.09 and any value between any of the foregoing.

201. The method of any of claims 1-200, wherein the modified SC-beta cell exhibits functionality for 1 or more days following transplantation into a subject.

202. The method of any of claims 1-201, wherein the modified SC-beta cell exhibits functionality for more than 1 week following transplantation into a subject.

203. The method of claim 201 or claim 202, wherein the functionality is selected from the group consisting of maintaining fasting blood glucose levels, secreting insulin in response to glucose injections in vivo, and clearing glucose after a glucose injection in vivo.

204. A composition comprising a population of modified SC-beta cells produced by the method of any of claims 1-203.

205. A modified stem-cell derived beta cell (SC-beta cell) comprising one or more modifications that:
(a) inactivate or disrupt one or more alleles of: (i) one or more major histocompatibility complex (MHC) class I molecules or one or more molecules that regulate expression of the one or more MHC class I molecules, and/or (ii) one or more MHC class II molecules or one or more molecules that regulate expression of the one or more MHC class II molecules, and/or
(b) increase expression of one or more tolerogenic factors, wherein the increased expression is relative to a control or wild-type beta cell that does not comprise the modifications.

206. The modified SC-beta cell of claim 205, wherein the modified SC-beta cell exhibits glucose-stimulated insulin secretion (GSIS).

207. A modified stem-cell derived beta cell (SC-beta cell) that has been differentiated in vitro from a pluripotent stem cell (PSC), wherein the modified SC-beta cell has modifications that
(a) inactivate or disrupt one or more alleles of: (i) one or more major histocompatibility complex (MHC) class I molecules or one or more molecules that regulate expression of the one or more MHC class I molecules, and/or (ii) one or more MHC class II molecules or one or more molecules that regulate expression of the one or more MHC class II molecules; and
(b) increase expression of one or more tolerogenic factors, relative to a control or wild-type beta cell, and wherein the modified SC-beta cell exhibits glucose-stimulated insulin secretion (GSIS).

208. The modified SC-beta cell of any of claims 205-207, wherein the tolerogenic factor is expressed at a first level that is greater than at or about 10-fold, greater than at or about 20-fold, greater than at or about 30-fold, greater than at or about 40-fold, greater than at or about 50-fold, greater than at or about 60-fold, or greater than at or about 70-fold over a second level expressed by the control or wild-type beta cell.

209. A modified stem cell-derived beta cell (SC-beta cell) that has been differentiated in vitro from a pluripotent stem cell (PSC), wherein the modified SC-beta cell (1) does not express one or more major histocompatibility complex (MHC) class I molecules and/or one or more MHC class II molecules, and (2) overexpresses a tolerogenic factor at a level that is greater than at or about 5-fold compared to expression of the tolerogenic factor by a control or wild-type beta cell, and wherein the modified SC-beta cell exhibits glucose-stimulated insulin secretion (GSIS).

210. The modified SC-beta cell of claim 209, wherein the expression of the tolerogenic factor is by flow cytometry with an antibody directed against the tolerogenic factor and the background is determined by flow cytometry staining with an isotype control of the antibody.

211. The modified SC-beta cell of claim 209 or claim 210, wherein the tolerogenic factor is expressed at a level that is greater than at or about 10-fold, greater than at or about 20-fold, greater than at or about 30-fold, greater than at or about 40-fold, greater than at or about 50-fold, greater than at or about 60-fold, or greater than at or about 70-fold compared to expression of the tolerogenic factor by a control or wild-type beta cell.

212. A modified stem cell-derived beta cell (SC-beta cell) that has been differentiated in vitro from a pluripotent stem cell (PSC), wherein the modified SC-beta cell (1) does not express one or more major histocompatibility complex (MHC) class I molecules and/or one or more MHC class II molecules and (2) overexpresses a tolerogenic factor at a first level of greater than at or about 5-fold over a second level expressed by a control or wild-type cell, wherein:
the control or wild-type cell is a control or wild-type PSC that does not comprise modifications to reduce one or more MHC class I molecules and/or one or more MHC class II molecules and to overexpress the tolerogenic factor or is a control or wild-type SC-beta cell differentiated from such control or wild-type PSC;
and wherein the modified SC-beta cell exhibits glucose-stimulated insulin secretion (GSIS).

213. The modified SC-beta cell of any of claims 205-212, wherein the tolerogenic factor is expressed by the modified SC-beta cell at greater than at or about 20,000 molecules per cell.

214. A modified stem cell-derived beta cell (SC-beta cell) that has been differentiated in vitro from a pluripotent stem cell (PSC), wherein the modified SC-beta cell (1) does not express one or more major histocompatibility complex (MHC) class I molecules and/or one or more MHC class II molecules and (2) overexpresses a tolerogenic factor, wherein the tolerogenic factor is expressed at greater than at or about 20,000 molecules per cell, and wherein the modified SC-beta cell exhibits glucose-stimulated insulin secretion (GSIS).

215. The modified SC-beta cell of any of claims 205-214, wherein the tolerogenic factor is expressed by the modified SC-beta cell at greater than at or about 30,000 molecules per cell, greater than at or about 50,000 molecules per cell, greater than at or about 100,000 molecules per cell, greater than at or about 200,000 molecules per cell, greater than at or about 300,000 molecules per cell, greater than at or about 400,000 molecules per cell, greater than at or about 500,000 molecules per cell, or greater than at or about 600,000 molecules per cell.

216. The modified SC-beta cell of any of claims 205-215, wherein the PSC is a modified PSC comprising one or more modifications selected from the group consisting of modifications that (a) inactivate or disrupt one or more alleles of: (i) one or more major histocompatibility complex (MHC) class I molecules or one or more molecules that regulate expression of the one or more MHC class I molecules, (ii) one or more MHC class II molecules or one or more molecules that regulate expression of the one or more MHC class II molecules; and (b) increase expression of a tolerogenic factor, relative to a control or wild-type PSC.

217. The modified SC-beta cell of claim 216, wherein the control or wild-type PSC is an PSC that does not comprise the one or more modifications.

218. The modified SC-beta cell of any of claims 205-207 and 214-217, wherein the modified SC-beta cell expresses the tolerogenic factor at a first level that is greater than at or about 5-fold over a second level expressed by the control or wild-type PSC or a control or wild-type SC-beta cell differentiated from the control or wild-type PSC.

219. The modified SC-beta cell of claim 218, wherein the tolerogenic factor is expressed at a first level that is greater than at or about 10-fold, greater than at or about 20-fold, greater than at or about 30-fold, greater than at or about 40-fold, greater than at or about 50-fold, greater than at or about 60-fold, or greater than at or about 70-fold over a second level expressed by the control or wild-type PSC or a control or wild-type SC-beta differentiated from the control or wild-type PSC.

220. A modified stem-cell derived beta cell (SC-beta cell) that has been differentiated in vitro from a modified pluripotent stem cell (PSC), wherein the modified PSC comprises one or more modifications selected from the group consisting of modifications that (a) inactivate or disrupt one or more alleles of: (i) one or more major histocompatibility complex (MHC) class I molecules or one or more molecules that regulate expression of the one or more MHC class I molecules, (ii) one or more MHC class II molecules or one or more molecules that regulate expression of the one or more MHC class II molecules; and (b) increase expression of a tolerogenic factor, relative to a control or wild-type PSC, and wherein the modified SC-beta cell exhibits glucose-stimulated insulin secretion (GSIS).

221. The modified SC-beta cell of claim 220, wherein the control or wild-type PSC is an PSC that does not comprise the modifications.

222. The modified SC-beta cell of any of claims 216-221, wherein the modified PSC expresses the tolerogenic factor at a first level that is greater than at or about 5-fold over a second level expressed by the control or wild-type PSC that does not comprise the one or more modifications, optionally wherein the tolerogenic factor is expressed at a first level that is greater than at or about 10-fold, greater than at or about 20-fold, greater than at or about 30-fold, greater than at or about 40-fold, greater than at or about 50-fold, greater than at or about 60-fold, or greater than at or about 70-fold over the second level expressed by the control or wild-type PSC that does not comprise the one or more modifications.

223. The modified SC-beta cell of any of claims 216-222, wherein the modified SC-beta cell comprises modifications that (a) inactivate or disrupt one or more alleles of: (i) one or more major histocompatibility complex (MHC) class I molecules or one or more molecules that regulate expression of the one or more MHC class I molecules, and/or (ii) one or more MHC class II molecules or one or more molecules that regulate expression of the one or more MHC class II molecules; and (b) increase expression of a tolerogenic factor, relative to a control or wild-type beta cell.

224. The modified SC-beta cell of claim 223, wherein the modified SC-beta cell expresses the tolerogenic factor at a first level that is greater than at or about 5-fold over a second level expressed by the control or wild-type PSC or the control or wild-type SC-beta cell differentiated from the control or wild-type PSC.

225. The modified SC-beta cell of claim 224, wherein the tolerogenic factor is expressed at a first level that is greater than at or about 10-fold, greater than at or about 20-fold, greater than at or about 30-fold, greater than at or about 40-fold, greater than at or about 50-fold, greater than at or about 60-fold, or greater than at or about 70-fold over a second level expressed by the control or wild-type PSC or the control or wild-type SC-beta cell differentiated from the control or wild-type PSC.

226. The modified SC-beta of any of claims 216-225, wherein the tolerogenic factor is expressed by the modified PSC at greater than at or about 20,000 molecules per cell.

227. A modified stem cell-derived beta cell (SC-beta cell) that has been differentiated in vitro from a modified pluripotent stem cell (PSC), wherein the modified PSC comprises modifications such that the modified PSC (a) does not express one or more major histocompatibility complex (MHC) class I molecules and/or or one or more MHC class II molecules; and (b) expresses a tolerogenic factor at greater than at or about 20,000 molecules per cell, and wherein the modified SC-beta cell exhibits glucose-stimulated insulin secretion (GSIS).

228. A modified stem cell-derived beta cell (SC-beta cell) that has been differentiated in vitro from a pluripotent stem cell (PSC), wherein the modified SC-beta cell comprises modifications such that the modified SC-beta cell (a) does not express one or more major histocompatibility complex (MHC) class I molecules and/or or one or more MHC class II molecules; and (b) expresses a tolerogenic factor at greater than at or about 20,000 molecules per cell, and wherein the modified SC-beta cell exhibits glucose-stimulated insulin secretion (GSIS).

229. The modified SC-beta cell of any of claims 226-227, wherein the tolerogenic factor is expressed by the modified PSC at greater than at or about 30,000 molecules per cell, greater than at or about 50,000 molecules per cell, greater than at or about 100,000 molecules per cell, greater than at or about 200,000 molecules per cell, greater than at or about 300,000 molecules per cell, greater than at or about 400,000 molecules per cell, greater than at or about 500,000 molecules per cell, or greater than at or about 600,000 molecules per cell.

230. The modified SC-beta cell of any of claims 226-229, wherein the tolerogenic factor is expressed by the modified SC-beta cell at greater than at or about 30,000 molecules per cell, greater than at or about 50,000 molecules per cell, greater than at or about 100,000 molecules per cell, greater than at or about 200,000 molecules per cell, greater than at or about 300,000 molecules per cell, greater than at or about 400,000 molecules per cell, greater than at or about 500,000 molecules per cell, or greater than at or about 600,000 molecules per cell.

231. The modified SC-beta cell of any of claims 216-230, wherein the modified SC-beta cell does not express MHC class I or MHC class II molecules and expresses the tolerogenic factor at greater than at or about 20,000 molecules per cell.

232. The modified SC-beta cell of claim 231, wherein the tolerogenic factor is expressed by the modified SC-beta cell at greater than at or about 30,000 molecules per cell, greater than at or about 50,000 molecules per cell, greater than at or about 100,000 molecules per cell, greater than at or about 200,000 molecules per cell, greater than at or about 300,000 molecules per cell, greater than at or about 400,000 molecules per cell, greater than at or about 500,000 molecules per cell, or greater than at or about 600,000 molecules per cell.

233. The modified SC-beta cell of any of claims 205-232, wherein the tolerogenic factor is selected from the group consisting of CD16, CD24, CD35, CD39, CD46, CD47, CD52, CD55, CD59, CD64, CD200, CCL22, CTLA4-Ig, C1 inhibitor, FASL, IDO1, HLA-C, HLA-E, HLA-E heavy chain, HLA-G, IL-10, IL-35, PD-L1, SERPINB9, CCL21, MFGE8, DUX4, B2M-HLA-E, CD27, IL-39, CD16 Fc Receptor, IL15-RF, H2-M3 (HLA-G), A20/TNFAIP3, CR1, HLA-F, MANF, and any combination thereof.

234. The modified SC-beta cell of any of claims 205-233, wherein the tolerogenic factor comprises CD47.

235. The modified SC-beta cell of any of claims 205-233, wherein the tolerogenic factor comprises PD-L1.

236. The modified SC-beta cell of any of claims 205-233, wherein the tolerogenic factor comprises HLA-E.

237. The modified SC-beta cell of any of claims 205-233, wherein the tolerogenic factor comprises HLA-G.

238. The modified SC-beta cell of any of claims 216-237, wherein the modifications in (a) reduce expression of one or more MHC class I molecules and/or one or more MHC class II molecules.

239. The modified SC-beta cell of any of claims 216-237, wherein expression of one or more MHC class I molecules and one or more MHC class II molecules is reduced in the modified PSC.

240. The modified SC-beta cell of any of claims 216-237, wherein the modifications in (a) reduce protein expression of one or more MHC class I molecules.

241. The modified SC-beta cell of any of claims 216-240, wherein the modifications in (a) reduce a function of the one or more MHC class I molecules, optionally wherein the function is antigen presentation.

242. The modified SC-beta cell of any of claims 216-241, wherein the one or more MHC class I molecules is one or more human leukocyte antigen (HLA) class I molecules.

243. The modified SC-beta cell of any of claims 216-242, wherein the one or more MHC HLA class I molecules is selected from the group consisting of HLA-A, HLA-B, and HLA-C.

244. The modified SC-beta cell of any of claims 216-243, wherein the one or more molecules that regulate expression of the one or more MHC class I molecules is selected from the group consisting of B2M, NLRC5 and TAP1.

245. The method of any of claims 216-244, wherein the one or more molecules that regulate expression of the one or more MHC class I molecules regulate cell surface protein expression of the one or more MHC class I molecules.

246. The method of claim 245, wherein the one or more molecules that regulate cell surface protein expression of the one or more MHC class I molecules are B2M.

247. The method of any of claims 216-246, wherein the one or more modifications comprise a modification that regulates expression of the one or more MHC class I molecules and the modification inactivates or disrupts one or more alleles of B2M.

248. The method of any of claims 216-247, wherein the one or more modifications in (a) reduce cell surface protein expression of the one or more MHC class I molecules.

249. The method of any of claims 216-248, wherein the one or more modifications in (a) reduce cell surface trafficking of the one or more MHC class I molecules.

250. The method of any of claims 216-249, wherein cell surface trafficking of the one or more MHC class I molecules and the one or more MHC class II molecules is reduced in the modified SC-beta cell relative to the control or wild-type SC-beta cell.

251. The modified SC-beta cell of any of claims 216-250, wherein the one or more molecules that regulate expression of the one or more MHC class I molecules regulate cell surface protein expression of the one or more MHC class I molecules.

252. The modified SC-beta cell of any of claims 244-251, wherein the modification that inactivates or disrupts one or more alleles of B2M reduces mRNA expression of the B2M gene.

253. The modified SC-beta cell of any of claims 244-252, wherein the modification that inactivates or disrupts one or more alleles of B2M reduces protein expression of B2M.

254. The modified SC-beta cell of any of claims 244-253, wherein the modification that inactivates or disrupts one or more alleles of B2M comprises:
inactivation or disruption of one allele of the B2M gene;
inactivation or disruption of both alleles of the B2M gene; or
inactivation or disruption of all B2M coding alleles in the cell.

255. The modified SC-beta cell of any of claims 244-254, wherein the inactivation or disruption comprises an indel in the B2M gene.

256. The modified SC-beta cell of any of claims 244-255, wherein the inactivation or disruption is a frameshift mutation or a deletion of a contiguous stretch of genomic DNA of the B2M gene.

257. The modified SC-beta cell of any of claims 205-256, wherein the modifications in (a) reduce protein expression of the one or more MHC class II molecules.

258. The modified SC-beta cell of any of claims 205-257, wherein the modifications in (a) reduce cell surface expression of the one or more MHC class II molecules.

259. The method of any of claims 205-258, wherein the one or more modifications in (a) reduce cell surface trafficking of the one or more MHC class II molecules.

260. The modified SC-beta cell of any of claims 205-259, wherein the modifications in (a) reduce a function of the one or more MHC class II molecules, optionally wherein the function is antigen presentation.

261. The modified SC-beta cell of any of claims 205-260, wherein the one or more MHC class II molecules is one or more human leukocyte antigen (HLA) class II molecules.

262. The modified SC-beta cell of any of claims 205-261, wherein the one or more MHC HLA class II molecules is selected from the group consisting of HLA-DP, HLA-DQ, and/or HLA-DR.

263. The modified SC-beta cell of any of claims 205-262, wherein one or more molecules that regulate expression of the one or more MHC class II molecules is selected from the group consisting of CIITA and CD74.

264. The modified SC-beta cell of any of claims 205-263, wherein the one or more modifications comprise a modification that regulates expression of the one or more MHC class II molecules, and the modification inactivates or disrupts one or more alleles of CIITA.

265. The modified SC-beta cell of any of claims 263-264, wherein the modification that inactivates or disrupts one or more alleles of CIITA reduces mRNA expression of the CIITA gene and/or wherein the modification that that inactivates or disrupts one or more alleles of one or more molecules that regulate expression of the one or more MHC class II molecules reduces protein expression of CIITA.

266. The modified SC-beta cell of any of claims 263-265, wherein the modification that inactivates or disrupts one or more alleles of CIITA comprises:
inactivation or disruption of one allele of the CIITA gene;
inactivation or disruption of both alleles of the CIITA gene; or
inactivation or disruption of all CIITA coding alleles in the cell.

267. The modified SC-beta cell of any of claims 263-266, wherein the inactivation or disruption comprises an indel in the CIITA gene.

268. The modified SC-beta cell of any of claims 263-267, wherein the inactivation or disruption is a frameshift mutation or a deletion of a contiguous stretch of genomic DNA of the CIITA gene.

269. The modified SC-beta cell of any of claims 216-268, wherein expression of HLA-A, HLA-B, HLA-C, HLA-DP, HLA-DQ, and HLA-DR are reduced in the modified PSC.

270. The modified SC-beta cell of any of claims 216-269, wherein the modified PSC comprises a modification that inactivates or disrupts one or more alleles of CD142.

271. The modified SC-beta cell of any of claims 205-270, wherein expression of HLA-A, HLA-B, HLA-C, HLA-DP, HLA-DQ, and HLA-DR are reduced in the modified SC-beta cell.

272. The modified SC-beta cell of any of claims 205-271, wherein the modified SC-beta cell comprises a modification inactivates or disrupts one or more alleles of CD142.

273. The modified SC-beta cell of any of claims 270-272, wherein the modification reduces mRNA expression of the CD142 gene.

274. The modified SC-beta cell of any of claims 270-273, wherein the modification reduces protein expression of CD142.

275. The modified SC-beta cell of any of claims 270-274, wherein the modification that inactivates or disrupts one or more alleles of CD142 comprises:
inactivation or disruption of one allele of the CD142 gene;
inactivation or disruption of both alleles of the CD142 gene; or
inactivation or disruption of all CD142 coding alleles in the cell.

276. The modified SC-beta cell of any of claims 270-275, wherein the inactivation or disruption comprises an indel in the CD142 gene.

277. The modified SC-beta cell of any of claims 270-276, wherein the inactivation or disruption is a frameshift mutation or a deletion of a contiguous stretch of genomic DNA of the CD142 gene.

278. The modified SC-beta cell of any of claims 205-277, wherein the modification to increase expression of the tolerogenic factor comprises an exogenous polynucleotide encoding the tolerogenic factor.

279. The modified SC-beta cell of claim 278, wherein the exogenous polynucleotide encoding the tolerogenic factor is integrated into the genome of the modified PSC.

280. The modified SC-beta cell of claim 279, wherein the exogenous polynucleotide encoding the tolerogenic factor is integrated by non-targeted insertion into the genome of the modified PSC.

281. The modified SC-beta cell of claim 279, wherein the exogenous polynucleotide encoding the tolerogenic factor is integrated by targeted insertion into a target genomic locus of the modified PSC.

282. The modified SC-beta cell of claim 278, wherein the exogenous polynucleotide encoding the tolerogenic factor is integrated into the genome of the modified SC-beta cell.

283. The modified SC-beta cell of claim 282, wherein the exogenous polynucleotide encoding the tolerogenic factor is integrated by non-targeted insertion into the genome of the modified SC-beta cell.

284. The modified SC-beta cell of claim 282, wherein the exogenous polynucleotide encoding the tolerogenic factor is integrated by targeted insertion into a target genomic locus of the modified SC-beta cell.

285. A modified stem cell derived beta cell (SC-beta cell) that has been differentiated in vitro from a pluripotent stem cell (PSC), wherein the modified SC-beta cell comprises knock out of the B2M gene, knock out of the CIITA gene, and an exogenous polynucleotide encoding exogenous CD47 protein.

286. The modified SC-beta cell of claim 285, wherein the PSC comprises one or more modifications selected from the group consisting of knock out of the B2M gene, knock out of the CIITA gene, and an exogenous polynucleotide encoding exogenous CD47 protein.

287. The modified SC-beta cell of claim 285, wherein the PSC does not comprise knock out of the B2M gene, knock out of the CIITA gene, or an exogenous polynucleotide encoding exogenous CD47 protein.

288. The modified SC-beta cell of any of claims 285-287, that has the phenotype B2M^{indel/indel}; CIITA^{indel/indel}; CD47tg.

289. A modified stem cell derived beta cell (SC-beta cell) that has been differentiated in vitro from a pluripotent stem cell (PSC), wherein the modified SC-beta cell comprises knock out of the B2M gene, knock out of the CIITA gene, an exogenous polynucleotide encoding CD47 protein, and an exogenous polynucleotide encoding a safety switch.

290. The modified SC-beta cell of claim 289, wherein the PSC comprises one or more modifications selected from the group consisting of knock out of the B2M gene, knock out of the CIITA gene, an exogenous polynucleotide encoding exogenous CD47 protein, and an exogenous polynucleotide encoding a safety switch.

291. The modified SC-beta cell of claim 289, wherein the PSC does not comprise knock out of the B2M gene, knock out of the CIITA gene, an exogenous polynucleotide encoding exogenous CD47 protein, or an exogenous polynucleotide encoding a safety switch.

292. The modified SC-beta cell of claim 216, wherein the modified SC-beta cell has the phenotype B2M^{indel/indel}; CIITA^{indel/indel}; CD47tg; safety switch transgene.

293. The modified SC-beta cell of any of claims 290-292, wherein the exogenous polynucleotide encoding CD47 is integrated by non-targeted insertion into the genome of the modified PSC.

294. The modified SC-beta cell of any of claims 285-292, wherein the exogenous polynucleotide encoding CD47 is integrated by non-targeted insertion into the genome of the modified SC-beta cell.

295. The modified SC-beta cell of any of claims 285-294, wherein the exogenous polynucleotide encoding CD47 is integrated by targeted insertion into a target genomic locus of the cell.

296. The modified SC-beta cell of any of claims 216-284, 294 and 295, wherein the modified PSC comprises an exogenous polynucleotide encoding a safety switch.

297. The modified SC-beta cell of claim 296, wherein the safety switch and the tolerogenic factor are expressed from a bicistronic cassette integrated into the genome of the modified PSC.

298. The modified SC-beta cell of claim 296 or claim 297, wherein the safety switch and CD47 are expressed from a bicistronic cassette integrated into the genome of the modified PSC.

299. The modified SC-beta cell of claim 297 or claim 298, wherein the bicistronic cassette is integrated by non-targeted insertion into the genome of the modified PSC.

300. The modified SC-beta cell of claim 297 or claim 298, wherein the bicistronic cassette is integrated by targeted insertion into a target genomic locus of the modified PSC.

301. The modified SC-beta cell of any of claims 205-284, 294 and 295, wherein the modified SC-beta cell comprises an exogenous polynucleotide encoding a safety switch.

302. The modified SC-beta cell of any of claim 289-301, wherein the safety switch is a system wherein upon activation, cells downregulate expression of the one or more tolerogenic factors and/or upregulate expression of one or more immune signaling molecules thereby marking the cell for elimination by the host immune system.

303. The modified SC-beta cell of claim 302, wherein the one or more tolerogenic factors are selected from the group consisting of CD16, CD24, CD35, CD39, CD46, CD47, CD52, CD55, CD59, CD64, CD200, CCL22, CTLA4-Ig, C1 inhibitor, FASL, IDO1, HLA-C, HLA-E, HLA-E heavy chain, HLA-G, IL-10, IL-35, PD-L1, SERPINB9, CCL21, MFGE8, DUX4, B2M-HLA-E, CD27, IL-39, CD16 Fc Receptor, IL15-RF, H2-M3 (HLA-G), A20/TNFAIP3, CR1, HLA-F, and MANF.

304. The modified SC-beta cell of claim 302 or claim 303, wherein the one or more immune signaling molecules are selected from the group consisting of B2M, HLA-A, HLA-B, HLA-C, HLA-D, HLA-E, RFXANK, CIITA, CTLA-4, PD-1, RAET1E/ULBP4, RAET1G/ULBP5, RAET1H/ULBP2, RAET1/ULBP1, RAET1L/ULBP6, RAET1N/ULBP3, and other ligands of NKG2D.

305. The modified SC-beta cell of any of claims 289-301, wherein the safety switch is a suicide gene.

306. The modified SC-beta cell of claim 305, wherein the suicide gene is selected from the group consisting of cytosine deaminase (CyD), herpesvirus thymidine kinase (HSV-Tk), an inducible caspase (iCaspase9), and rapamycin-activated caspase 9 (rapaCasp9).

307. The modified SC-beta cell of any of claims 301-306, wherein the safety switch and the tolerogenic factor are expressed from a bicistronic cassette integrated into the genome of the modified SC-beta cell.

308. The modified SC-beta cell of any one of claims 301-307, wherein the safety switch and CD47 are expressed from a bicistronic cassette integrated into the genome of the modified SC-beta cell.

309. The modified SC-beta cell of claim 307 or claim 308, wherein the bicistronic cassette is integrated by non-targeted insertion into the genome of the modified SC-beta cell.

310. The modified SC-beta cell of claim 307 or claim 308, wherein the bicistronic cassette is integrated by targeted insertion into a target genomic locus of the modified SC-beta cell.

311. The modified SC-beta cell of claim 281, claim 284, claim 295, claim 300, or claim 310, wherein the target genomic locus is a safe harbor locus, a B2M gene locus, a CIITA gene locus, or a CD142 gene locus.

312. The modified SC-beta cell of claim 311, wherein the safe harbor locus is selected from the group consisting of: a CCR5 gene locus, a CXCR4 gene locus, a PPP1R12C (also known as AAVS1) gene, an albumin gene locus, a SHS231 locus, a CLYBL gene locus, and a ROSA26 gene locus.

313. The modified SC-beta cell of any of claims 216-312, wherein the modified PSC comprises a modification that increases expression of one or more complement inhibitors selected from the group consisting of CD46, CD59, CD55, and CD35 relative to the control or wild-type PSC.

314. The modified SC-beta cell of any of claims 205-313, wherein the modified SC-beta cell comprises a modification that increases expression of one or more complement inhibitors selected from the group consisting of CD46, CD59, CD55, and CD35 relative to the control or wild-type SC-beta cell.

315. The modified SC-beta cell of claim 313 or claim 314, wherein the modification to increase expression of one or more complement inhibitors comprises at least one exogenous polynucleotide encoding one or more complement inhibitors selected from the group consisting of an exogenous polynucleotide encoding CD46, an exogenous polynucleotide encoding CD59, an exogenous polynucleotide encoding CD55, and an exogenous polynucleotide encoding CD35.

316. The modified SC-beta cell of any of claims 313-315, wherein the one or more complement inhibitors are CD46 and CD59.

317. The modified SC-beta cell of any of claims 313-315, wherein the one or more complement inhibitors are CD46, CD59 and CD55.

318. The modified SC-beta cell of any of claims 315-317, wherein the at least one exogenous polynucleotide encoding the one or more complement inhibitors is integrated by non-targeted insertion into the genome of the modified PSC.

319. The modified SC-beta cell of any of claims 315-317, wherein the at least one exogenous polynucleotide encoding the one or more complement inhibitors is integrated by non-targeted insertion into the genome of the modified SC-beta cell.

320. The modified SC-beta cell of any of claims 315-319, wherein the at least one exogenous polynucleotide encoding the one or more complement inhibitors is integrated by targeted insertion into a target genomic locus of the cell.

321. The modified SC-beta cell of claim 320, wherein the target genomic locus is a safe harbor locus, a B2M gene locus, a CIITA gene locus, or a CD142 gene locus.

322. The modified SC-beta cell of claim 321, wherein the safe harbor locus is selected from the group consisting of: a CCR5 gene locus, a CXCR4 gene locus, a PPP1R12C (also known as AAVS1) gene, an albumin gene locus, a SHS231 locus, a CLYBL gene locus, and a ROSA26 gene locus.

323. The modified SC-beta cell of any of claims 205-322, wherein the modifications in (a) reduce expression of one or more MHC class I molecules and/or one or more MHC class II molecules.

324. The modified SC-beta cell of any of claims 205-323, wherein the expression of the one or more MHC class I molecules and the one or more MHC class II molecules is reduced in the modified SC-beta cell.

325. The modified SC-beta cell of any of claim 205-324, wherein the modifications in (a) reduce cell surface protein expression of the one or more MHC class I molecules in the modified SC-beta cell.

326. The method of any of claims 205-324, wherein the one or more modifications in (a) reduce cell surface trafficking of the one or more MHC class I molecules.

327. The modified SC-beta cell of any of claims 205-326, wherein the modifications in (a) reduce a function of the one or more MHC class I molecules in the modified SC-beta cell, optionally wherein the function is antigen presentation.

328. The modified SC-beta cell of any of claims 205-327, wherein the one or more modifications comprise a modification that regulates expression of the one or more MHC class I molecules and the modification inactivates or disrupts one or more alleles of B2M in the modified SC-beta cell.

329. The modified SC-beta cell of claim 328, wherein the modification that inactivates or disrupts one or more alleles of B2M in the modified SC-beta cell reduces mRNA expression of the B2M gene.

330. The modified SC-beta cell of claim 328 or claim 329, wherein the modification that inactivates or disrupts one or more alleles of B2M in the modified SC-beta cell reduces protein expression of B2M.

331. The modified SC-beta cell of any of claims 328-330, wherein the modification that inactivates or disrupts one or more alleles of B2M in the modified SC-beta cell comprises:
inactivation or disruption of one allele of the B2M gene;
inactivation or disruption of both alleles of the B2M gene; or
inactivation or disruption of all B2M coding alleles in the cell.

332. The modified SC-beta cell of any of any of claims 328-331, wherein the inactivation or disruption comprises an indel in the B2M gene.

333. The modified SC-beta cell of any of claims 328-332, wherein the inactivation or disruption is a frameshift mutation or a deletion of a contiguous stretch of genomic DNA of the B2M gene.

334. The modified SC-beta cell of any of claims 205-333, wherein the one or more modifications in (a) reduce cell surface expression of the one or more MHC class II molecules.

335. The method of any of claims 205-334, wherein the one or more modifications in (a) reduce cell surface trafficking of the one or more MHC class II molecules.

336. The modified SC-beta cell of any of claims 205-335, wherein the one or more modifications in (a) reduce a function of the one or more MHC class II molecules, optionally wherein the function is antigen presentation.

337. The modified SC-beta cell of any of claims 205-336, wherein the one or more modifications comprise a modification that regulates expression of the one or more MHC class II molecules and the modification inactivates or disrupts one or more alleles of CIITA.

338. The modified SC-beta cell of claim 337, wherein the modification that inactivates or disrupts one or more alleles of CIITA in the modified SC-beta cell reduces mRNA expression of the CIITA gene or wherein the modification that inactivates or disrupts one or more alleles of one or more molecules that regulate expression of the one or more MHC class II molecules in the modified SC-beta cell reduces protein expression of CIITA.

339. The modified SC-beta cell of any of claims 337-338, wherein the modification that inactivates or disrupts one or more alleles of one or more MHC class II molecules or one or more molecules that regulate expression of the one or more MHC class II molecules comprises:
inactivation or disruption of one allele of the CIITA gene;
inactivation or disruption of both alleles of the CIITA gene; or
inactivation or disruption of all CIITA coding alleles in the cell.

340. The modified SC-beta cell of any of claims 337-339, wherein the inactivation or disruption comprises an indel in the CIITA gene.

341. The modified SC-beta cell of any of claims 337-340, wherein the inactivation or disruption is a frameshift mutation or a deletion of a contiguous stretch of genomic DNA of the CIITA gene.

342. The modified SC-beta cell of any of claims 205-341, wherein expression of HLA-A, HLA-B, HLA-C, HLA-DP, HLA-DQ, and HLA-DR are reduced in the modified SC-beta cell.

343. The modified SC-beta cell of any of claims 205-342, wherein the modified SC-beta cell comprises a modification that inactivates or disrupts an allele of CD142.

344. The modified SC-beta cell of claim 343, wherein the modification reduces mRNA expression of the CD142 gene, relative to a control or wild-type beta cell.

345. The modified SC-beta cell of claim 343 or claim 344, wherein the modification reduces protein expression of CD142, relative to a control or wild-type beta cell.

346. The modified SC-beta cell of any of claims 343-345, wherein the modification that inactivates or disrupts one or more alleles of CD142 in the modified SC-beta cell comprises:
inactivation or disruption of one allele of the CD142 gene;
inactivation or disruption of both alleles of the CD142 gene; or
inactivation or disruption of all CD142 coding alleles in the cell.

347. The modified SC-beta cell of any of claims 343-346, wherein the inactivation or disruption comprises an indel in the CD142 gene.

348. The modified SC-beta cell of any of claims 343-347, wherein the inactivation or disruption comprises a frameshift mutation or a deletion of a contiguous stretch of genomic DNA of the CD142 gene.

349. The modified SC-beta cell of any of claims 205-348, wherein the modification to increase expression of the tolerogenic factor in the modified SC-beta cell comprises an exogenous polynucleotide encoding the tolerogenic factor.

350. The modified SC-beta cell of claim 349, wherein the exogenous polynucleotide encoding the tolerogenic factor is integrated into the genome of the modified SC-beta cell.

351. The modified SC-beta cell of claim 350, wherein the exogenous polynucleotide is integrated into a non-target locus in the genome of the modified SC-beta cell.

352. The modified SC-beta cell of claim 350, wherein the exogenous polynucleotide is integrated into a target genomic locus of the modified SC-beta cell.

353. The modified SC-beta cell of any of claims 205-352, wherein the modified SC-beta cell further comprises a modification for expression of an exogenous suicide gene selected from the group consisting of cytosine deaminase (CyD), herpesvirus thymidine kinase (HSV-Tk), an inducible caspase (iCaspase9), and rapamycin-activated caspase 9 (rapaCasp9).

354. The modified SC-beta cell of claim 353, wherein the suicide gene and the tolerogenic factor are expressed from a bicistronic cassette integrated into the genome of the modified SC-beta cell.

355. The modified SC-beta cell of claim 354, wherein the bicistronic cassette is integrated at a non-target locus in the genome of the modified SC-beta cell.

356. The modified SC-beta cell of claim 354, wherein the bicistronic cassette is integrated into a target genomic locus of the cell.

357. The modified SC-beta cell of claim 352 or claim 356, wherein the target genomic locus is a safe harbor locus, a B2M gene locus, a CIITA gene locus, or a CD142 gene locus.

358. The modified SC-beta cell of claim 357, wherein the safe harbor locus is selected from the group consisting of: a CCR5 gene locus, a CXCR4 gene locus, a PPP1R12C (also known as AAVS1) gene, an albumin gene locus, a SHS231 locus, a CLYBL gene locus, and a ROSA26 gene locus.

359. The modified SC-beta cell of any of claims 205-358, wherein the modified SC-beta cell comprises a modification that increases expression of one or more complement inhibitors selected from the group consisting of CD46, CD59, CD55, and CD35 relative to a control or wild-type beta cell.

360. The modified SC-beta cell of claim 359, wherein the modification to increase expression of the one or more complement inhibitors in the modified SC-beta cell comprises at least one exogenous polynucleotide encoding one or more complement inhibitors selected from the group consisting of an exogenous polynucleotide encoding CD46, an exogenous polynucleotide encoding CD59, an exogenous polynucleotide encoding CD55, and an exogenous polynucleotide encoding CD35.

361. The modified SC-beta cell of claim 359 or claim 360, wherein the one or more complement inhibitors are CD46 and CD59.

362. The modified SC-beta cell of claim 359 or claim 360, wherein the one or more complement inhibitors are CD46, CD59 and CD55.

363. The modified SC-beta cell of any of claims 205-362, wherein the tolerogenic factor is CD47 and the modified SC-beta cell expresses CD47 at a first level that is greater than at or about 5-fold over a second level expressed by the control or wild-type beta cell.

364. The modified SC-beta cell of claim 363, wherein CD47 is expressed at a first level that is greater than at or about 10-fold, greater than at or about 20-fold, greater than at or about 30-fold, greater than at or about 40-fold, greater than at or about 50-fold, greater than at or about 60-fold, or greater than at or about 70-fold over a second level expressed by the control or wild-type beta cell.

365. The modified SC-beta cell of any of claims 205-364, wherein the tolerogenic factor is CD47 and CD47 is expressed by the modified SC-beta cell at greater than at or about 20,000 molecules per cell.

366. The modified SC-beta cell of claim 365, wherein CD47 is expressed by the modified SC-beta cell at greater than at or about 30,000 molecules per cell, greater than at or about 50,000 molecules per cell, greater than at or about 100,000 molecules per cell, greater than at or about 200,000 molecules per cell, greater than at or about 300,000 molecules per cell, greater than at or about 400,000 molecules per cell, greater than at or about 500,000 molecules per cell, or greater than at or about 600,000 molecules per cell.

367. The modified SC-beta cell of any of claims 205-366, wherein the control or wild-type beta cell is a SC-beta cell differentiated from a control or wild-type PSC not comprising modifications that (a) inactivate or disrupt one or more alleles of: (i) inactivate or disrupt one or more alleles of: (i) one or more major histocompatibility complex (MHC) class I molecules or one or more molecules that regulate expression of the one or more MHC class I molecules, and/or (ii) one or more MHC class II molecules or one or more molecules that regulate expression of the one or more MHC class II molecules and (b) that increase expression of the tolerogenic factor or is a wild-type primary beta cell.

368. The modified SC-beta of any of claims 205-367, wherein the modified SC-beta cell expresses at least one beta cell marker, optionally wherein the beta cell marker is selected from the group consisting of INS, CHGA, NKX2-2, PDX1, NKX6-1, MAFB, GCK and GLUT1.

369. The modified SC-beta cell of any of claims 205-368, wherein the modified SC-beta cell exhibits one or more functions of a wild-type or control beta cell, optionally wherein the one or more functions is selected from the group consisting of in vitro glucose-stimulated insulin secretion (GSIS), glucose metabolism, maintaining fasting blood glucose levels, secreting insulin in response to glucose injections in vivo, and clearing glucose after a glucose injection in vivo.

370. The modified SC-beta cell of any of claims 205-369, wherein the GSIS is measured in a perfusion GSIS assay.

371. The modified SC-beta cell of any of claims 205-370, wherein the GSIS is dynamic GSIS comprising first and second phase dynamic insulin secretion.

372. The modified SC-beta cell of any of claims 205-371, wherein the GSIS is static GSIS, optionally wherein the static stimulation index is greater than at or about 1, greater than at or about 1.5, greater than at or about 2, greater than at or about 5, greater than at or about 10, greater than at or about 15, or greater than at or about 20.

373. The modified SC-beta cell of any of claims 205-372, wherein the level of insulin secretion by the modified SC-beta cells is at least 20% of that observed for primary beta islets, optionally cadaveric islets.

374. The modified SC-beta cell of any of claims 205-373, wherein the level of insulin secretion by the modified SC-beta cells is at least 25%, at least 30%, at least 40%, at least 50%, at least 60%, at least 70% of that observed for primary beta islets, optionally cadaveric islets.

375. The modified SC-beta cell of any of claims 205-374, wherein the total insulin content of the modified SC-beta is greater than at or about 500 μIU Insulin per 5000 cells, greater than at or about 1000 μIU Insulin per 5000 cells, greater than at or about 2000 μIU Insulin per 5000 cells, greater than at or about 3000 μIU Insulin per 5000 cells or greater than at or about 4000 μIU Insulin per 5000 cells.

376. The modified SC-beta cell of any of claims 205-375, wherein the proinsulin to insulin ratio of the modified SC-beta is between at or about 0.02 and at or about 0.1, optionally at or about 0.03, 0.04, 0.05, 0.06, 0.07, 0.08, 0.09, and any value between any of the foregoing.

377. The modified SC-beta cell of any of claims 205-376, wherein the modified SC-beta cell exhibits functionality for 1 or more days following transplantation into a subject.

378. The modified SC-beta cell of any of claims 205-377, wherein the modified SC-beta cell exhibits functionality for more than 1 week following transplantation into a subject.

379. The modified SC-beta cell of claim 377 or claim 378, wherein the functionality is selected from the group consisting of maintaining fasting blood glucose levels, secreting insulin in response to glucose injections in vivo, and clearing glucose after a glucose injection in vivo.

380. A composition comprising a modified SC-beta cell of any of claims 205-379.

381. A composition comprising a population of modified SC-beta cells of any of claims 205-380.

382. The composition of claim 204 or claim 381, wherein, among the cells in the population, the level of expression of MHC class I molecules and/or MHC class II molecules and/or the level of the increased expression of the tolerogenic factor is retained or is similar compared to the modified PSC in at least at or about 50%, at least at or about 60%, at least at or about 70%, at least at or about 80%, or at least at or about 90% of the cells in the population.

383. The composition of claim 204, claim 381 or claim 382, wherein at least at or about 50%, at least at or about 60%, at least at or about 70%, at least at or about 80%, or at least at or about 90% of the cells in the population are reduced for expression of one or more MHC class I molecules and/or for expression of B2M.

384. The composition of any of claim 204 or 381-383, wherein at least at or about 50%, at least at or about 60%, at least at or about 70%, at least at or about 80%, or at least at or about 90% of the cells in the population are reduced for expression of one or more MHC class II molecules and/or for expression of CIITA.

385. The composition of claim 204, claim 381-384, wherein at least at or about 50%, at least at or about 60%, at least at or about 70%, at least at or about 80%, or at least at or about 90% of the cells in the population comprise inactivation or disruption of one or more alleles of: one or more MHC class I molecules and/or B2M.

386. The composition of any of claim 204 or 381-385, wherein at least at or about 50%, at least at or about 60%, at least at or about 70%, at least at or about 80%, or at least at or about 90% of the cells in the population comprise inactivation or disruption of one or more alleles of: one or more MHC class II molecules and/or CIITA.

387. The composition of any of claim 204 or 381-386, wherein at least at or about 50%, at least at or about 60%, at least at or about 70%, at least at or about 80%, or at least at or about 90% of the cells in the population express the tolerogenic factor at a first level that is greater than at or about 5-fold, greater than at or about 10-fold, greater than at or about 20-fold, greater than at or about 30-fold, greater than at or about 40-fold, greater than at or about 50-fold, greater than at or about 60-fold, or greater than at or about 70-fold over a second level expressed by the control or wild-type beta cell, optionally wherein the control or wild-type beta cell is a wild-type primary beta cell.

388. The composition of any of claim 204 or 381-387, wherein at least at or about 50%, at least at or about 60%, at least at or about 70%, at least at or about 80%, or at least at or about 90% of the cells in the population express the tolerogenic factor at a first level that is greater than at or about 5-fold, greater than at or about 10-fold, greater than at or about 20-fold, greater than at or about 30-fold, greater than at or about 40-fold, greater than at or about 50-fold, greater than at or about 60-fold, or greater than at or about 70-fold over a second level expressed by a control or wild-type PSC not comprising the modifications or a control or wild-type SC-beta cell differentiated from the control or wild-type PSC.

389. The composition of any of claim 204 or 381-388, wherein at least at or about 50%, at least at or about 60%, at least at or about 70%, at least at or about 80%, or at least at or about 90% of the cells in the population expresses the tolerogenic factor at greater than at or about 20,000 molecules per cell, at greater than at or about 30,000 molecules per cell, greater than at or about 50,000 molecules per cell, greater than at or about 100,000 molecules per cell, greater than at or about 200,000 molecules per cell, greater than at or about 300,000 molecules per cell, greater than at or about 400,000 molecules per cell, greater than at or about 500,000 molecules per cell, or greater than at or about 600,000 molecules per cell.

390. The composition of any of claim 204 or 381-389, wherein at least at or about 50%, at least at or about 60%, at least at or about 70%, at least at or about 80%, or at least at or about 90% of the cells in the population comprise one or more modifications that inactivate or disrupt CD142.

391. The modified SC-beta cell of any of claims 205-380 or the composition of any of claims 204 and 381-389, wherein the inactivation or disruption is by one or more gene edits.

392. The modified SC-beta cell or the composition of claim 391, wherein the cell comprises a genome editing complex.

393. The modified SC-beta cell or the composition of claim 391, wherein the one or more gene edits are made by a genome editing complex.

394. The modified SC-beta cell or composition according to claim 392 or claim 393, wherein the genome editing complex comprises a genome targeting entity and a genome modifying entity.

395. The modified SC-beta cell or composition according to claim 394, wherein the genome targeting entity localizes the genome editing complex to the one or more alleles that are inactivated or disrupted, optionally wherein the genome targeting entity is a nucleic acid-guided targeting entity.

396. The modified SC-beta cell or composition according to claim 394 or claim 395, wherein the genome targeting entity is selected from the group consisting of a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising the gRNA and the Cas nuclease, a homing endonuclease, a zinc finger nuclease (ZF) nucleic acid binding entity, a transcription activator-like effector (TALE) nucleic acid binding entity, a meganuclease, a Cas nuclease, a core Cas protein, a homing endonuclease, an endonuclease-deficient-Cas protein, an enzymatically inactive Cas protein, a CRISPR-associated transposase (CAST), a Type II or Type V Cas protein, or a functional portion thereof.

397. The modified SC-beta cell or composition according to any one of claims 394-396, wherein the genome targeting entity is selected from the group consisting of Cas1, Cas2, Cas3, Cas4, Cas5, Cas6, Cas7, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr1, Cmr2, Cmr3, Cmr4, Cmr5, Cmr6, Csd1, Csd2, Cas5d, Cse1, Cse2, Cse3, Cse4, Cas5e, Csf1, Csm1, Csm2, Csm3, Csm4, Csm5, Csn1, Csn2, Cst1, Cst2, Cas5t, Csh1, Csh2, Cas5h, Csa1, Csa2, Csa3, Csa4, Csa5, Cas5a, Csx10, Csx11, Csy1, Csy2, Csy3, Csy4, Mad7, SpCas9, eSpCas9, SpCas9-HF1, HypaSpCas9, HeFSpCas9, and evoSpCas9 high-fidelity variants of SpCas9, SaCas9, NmeCas9, CjCas9, StCas9, TdCas9, LbCas12a, AsCas12a, AacCas12b, BhCas12b v4, TnpB, dCas (D10A), dCas (H840A), dCas13a, dCas13b, or a functional portion thereof.

398. The modified SC-beta cell or composition according to any of claims 394-397, wherein the genome modifying entity cleaves, deaminates, nicks, polymerizes, interrogates, integrates, cuts, unwinds, breaks, alters, methylates, demethylates, or otherwise destabilizes the target locus.

399. The modified SC-beta cell or composition according to any of claims 394-398, wherein the genome modifying entity comprises a recombinase, integrase, transposase, endonuclease, exonuclease, nickase, helicase, DNA polymerase, RNA polymerase, reverse transcriptase, deaminase, flippase, methylase, demethylase, acetylase, a nucleic acid modifying protein, an RNA modifying protein, a DNA modifying protein, an Argonaute protein, an epigenetic modifying protein, a histone modifying protein, or a functional portion thereof.

400. The modified SC-beta cell or composition according to any one of claims 394-399, wherein the genome modifying entity is selected from the group consisting of a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising the gRNA and the Cas nuclease, a homing endonuclease, a zinc finger nuclease (ZFN), a transcription activator-like effector nuclease (TALEN), a meganuclease, a Cas nuclease, a core Cas protein, a homing endonuclease, an endonuclease-deficient-Cas protein, an enzymatically inactive Cas protein, a CRISPR-associated transposase (CAST), a Type II or Type V Cas protein, base editing, prime editing, a Programmable Addition via Site-specific Targeting Elements (PASTE), or a functional portion thereof.

401. The modified SC-beta cell or composition according to any one of claims 394-400, wherein the genome modifying entity is selected from the group consisting of Cas1, Cas2, Cas3, Cas4, Cas5, Cas6, Cas7, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr1, Cmr2, Cmr3, Cmr4, Cmr5, Cmr6, Csd1, Csd2, Cas5d, Cse1, Cse2, Cse3, Cse4, Cas5e, Csf1, Csm1, Csm2, Csm3, Csm4, Csm5, Csn1, Csn2, Cst1, Cst2, Cas5t, Csh1, Csh2, Cas5h, Csa1, Csa2, Csa3, Csa4, Csa5, Cas5a, Csx10, Csx11, Csy1, Csy2, Csy3, Csy4, Mad7, SpCas9, eSpCas9, SpCas9-HF1, HypaSpCas9, HeFSpCas9, and evoSpCas9 high-fidelity variants of SpCas9, SaCas9, NmeCas9, CjCas9, StCas9, TdCas9, LbCas12a, AsCas12a, AacCas12b, BhCas12b v4, TnpB, FokI, dCas (D10A), dCas (H840A), dCas13a, dCas13b, a base editor, a prime editor (e.g., a target-primed reverse transcription (TPRT) editor), APOBEC1, cytidine deaminase, adenosine deaminase, uracil glycosylase inhibitor (UGI), adenine base editors (ABE), cytosine base editors (CBE), reverse transcriptase, serine integrase, recombinase, transposase, polymerase, adenine-to-thymine or “ATBE” (or thymine-to-adenine or “TABE”) transversion base editor, ten-eleven translocation methylcytosine dioxygenases (TETs), TET1, TET3, TET1CD, histone acetyltransferase p300, histone methyltransferase SMYD3, histone methyltransferase PRDM9, H3K79 methyltransferase DOT1L, transcriptional repressor, or a functional portion thereof.

402. The modified SC-beta cell or composition according to any one of claims 394-401, wherein the genome targeting entity and the genome modifying entity are different domains of a single polypeptide.

403. The modified SC-beta cell or composition according to any one of claims 394-402, wherein the genome editing entity and genome modifying entity are two different polypeptides that are operably linked together.

404. The modified SC-beta cell or composition according to any one of claims 394-402, wherein the genome editing entity and genome modifying entity are two different polypeptides that are not linked together.

405. The modified SC-beta cell or composition according to any one of claims 394-402, wherein the genome editing complex comprises a guide nucleic acid having a targeting domain that is complementary to at least one target locus, optionally wherein the guide nucleic acid is a guide RNA (gRNA).

406. The modified SC-beta cell or composition according to any one of claims 394-402, wherein the one or more modifications are made by the genome editing complex.

407. The modified SC-beta cell or composition according to claim 406, wherein the one or more modifications made by the genome editing complex are made by a sequence specific nuclease, a nucleic acid programmable DNA binding protein, an RNA guided nuclease, RNA-guided nuclease comprising a Cas nuclease and a guide RNA (CRISPR-Cas combination), a ribonucleoprotein (RNP) complex comprising the gRNA and the Cas nuclease, a homing endonuclease, a zinc finger nuclease (ZFN), a transcription activator-like effector nuclease (TALEN), a meganuclease, a Cas nuclease, a core Cas protein, a TnpB nuclease, a homing endonuclease, an endonuclease-deficient-Cas protein, an enzymatically inactive Cas protein, a CRISPR-associated transposase (CAST), a Type II or Type V Cas protein, base editing, prime editing, or a Programmable Addition via Site-specific Targeting Elements (PASTE).

408. The modified SC-beta cell or composition according to claim 406 or claim 407, wherein the one or more modifications made by the genome editing complex are made by Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, a CRISPR-associated transposase, base editing, prime editing, or Programmable Addition via Site-specific Targeting Elements (PASTE).

409. The modified SC-beta cell or composition according to any one of claims 406-408, wherein the modifications made by the genome editing complex are made using a guide RNA (gRNA) having a targeting domain that is complementary to at least one target site.

410. The modified SC-beta cell or composition of any of claims 391-393, wherein the genome editing complex is an RNA-guided nuclease.

411. The modified SC-beta cell or composition of claim 410, wherein the RNA-guided nuclease comprises a Cas nuclease and a guide RNA (CRISPR-Cas combination).

412. The modified SC-beta cell or composition of claim 411, wherein the CRISPR-Cas combination is a ribonucleoprotein (RNP) complex comprising the gRNA and the Cas nuclease.

413. The modified SC-beta cell or composition of claim 411 or claim 412, wherein the Cas nuclease is a Type II or Type V Cas protein.

414. The modified SC-beta cell or composition of any of claims 411-413, wherein the genome-modifying protein is selected from the group consisting of Cas3, Cas4, Cas5, Cas8a, Cas8b, Cas8c, Cas9, Cas10, Cas12, Cas12a (Cpf1), Cas12b (C2c1), Cas12c (C2c3), Cas12d (CasY), Cas12e (CasX), Cas12f (C2c10), Cas12g, Cas12h, Cas12i, Cas12k (C2c5), Cas13, Cas13a (C2c2), Cas13b, Cas13c, Cas13d, C2c4, C2c8, C2c9, Cmr5, Cse1, Cse2, Csf1, Csm2, Csn2, Csx10, Csx11, Csy1, Csy2, Csy3, Mad7, a zinc finger nuclease (ZFN), a transcription activator-like effector nuclease (TALEN), a meganuclease, and a CRISPR-associated transposase, or a homologue of any of the foregoing.

415. The composition of any of claims 204 and 381-414 comprising a pharmaceutically acceptable excipient.

416. The composition of any of claims 204 and 381-415 comprising a cryoprotectant.

417. A method of treating diabetes in a subject, the method comprising administering the modified SC-beta cells of any of claims 205-380 or the composition of any of claims 204 and 381-416 to a subject in need thereof.

418. The method of claim 417, wherein the diabetes is type I diabetes.

419. The method of claim 418, wherein the diabetes is type II diabetes.

420. The method of any of claims 417-419, wherein the modified SC-beta cells improve glucose tolerance in the subject.

421. A method for improving glucose tolerance in a subject, the method comprising administering the modified SC-beta cells of any of claims 205-380 or the composition of any of claims 204 and 381-416 to a subject in need thereof.

422. The method of any of claims 417-421, wherein the subject is a diabetic patient.

423. The method of claim 422, wherein the diabetic patient has type I diabetes or type II diabetes.

424. The method of any of claims 417-423, wherein glucose tolerance is improved relative to the subject's glucose tolerance prior to administration of the modified SC-beta cells.

425. The method of any of claims 417-424, wherein administration of the modified SC-beta cells reduces exogenous insulin usage in the subject.

426. The method of any of claims 417-425, wherein glucose tolerance is improved as measured by HbA1c levels.

427. The method of any of claims 417-426, wherein the subject is fasting.

428. The method of any one of claims 417-427, wherein administration of the modified SC-beta cells improves insulin secretion in the subject.

429. The method of claim 428, wherein insulin secretion is improved relative to the subject's insulin secretion prior to administration of the modified SC-beta cells.

430. The method of any of claims 417-429, further comprising administering one or more immunosuppressive agents to the subject.

431. The method of any of claims 417-429, wherein the subject has been administered one or more immunosuppressive agents.

432. The method of claim 430 or 431, wherein the one or more immunosuppressive agents are a small molecule or an antibody.

433. The method of any of claims 430-432, wherein the one or more immunosuppressive agents are selected from the group consisting of cyclosporine, azathioprine, mycophenolic acid, mycophenolate mofetil, a corticosteroids, prednisone, methotrexate, gold salts, sulfasalazine, antimalarials, brequinar, leflunomide, mizoribine, 15-deoxyspergualine, 6-mercaptopurine, cyclophosphamide, rapamycin, tacrolimus (FK-506), OKT3, anti-thymocyte globulin, thymopentin (thymosin-α), and an immunosuppressive antibody.

434. The method of any of claims 430-433, wherein the one or more immunosuppressive agents comprise cyclosporine.

435. The method of any of claims 430-433, wherein the one or more immunosuppressive agents comprise mycophenolate mofetil.

436. The method of any of claims 430-433, wherein the one or more immunosuppressive agents comprise a corticosteroid.

437. The method of any of claims 430-433, wherein the one or more immunosuppressive agents comprise cyclophosphamide.

438. The method of any of claims 430-433, wherein the one or more immunosuppressive agents comprise rapamycin.

439. The method of any of claims 430-433, wherein the one or more immunosuppressive agents comprise tacrolimus (FK-506).

440. The method of any of claims 430-433, wherein the one or more immunosuppressive agents comprise anti-thymocyte globulin.

441. The method of any of claims 430-433, wherein the one or more immunosuppressive agents are one or more immunomodulatory agents.

442. The method of claim 441, wherein the one or more immunomodulatory agents are a small molecule or an antibody.

443. The method of claim 432 or claim 442, wherein the antibody binds to one or more of receptors or ligands selected from the group consisting of p75 of the IL-2 receptor, MHC, CD2, CD3, CD4, CD7, CD28, B7, CD40, CD45, IFN-gamma, TNF-alpha, IL-4, IL-5, IL-6R, IL-6, IGF, IGFR1, IL-7, IL-8, IL-10, CD11a, CD58, and antibodies binding to any of their ligands.

444. The method of any of claims 430-443, wherein the one or more immunosuppressive agents are or have been administered to the subject prior to administration of the modified SC-beta cells.

445. The method of any of claims 430-444, wherein the one or more immunosuppressive agents are or have been administered to the subject at least 1, 2, 3, 4, 5, 6, 7, 8, 9, 10, 11, 12, 13, or 14 days prior to administration of the modified SC-beta cells.

446. The method of any of claims 430-444, wherein the one or more immunosuppressive agents are or have been administered to the subject at least 1 week, 2 weeks, 3 weeks, 4 weeks, 5 weeks, 6 weeks, 7 weeks, 8 weeks, 9 weeks, 10 weeks or more prior to administration of the modified SC-beta cells.

447. The method of any of claims 430-444, wherein the one or more immunosuppressive agents are or have been administered to the subject at least 1, 2, 3, 4, 5, 6, 7, 8, 9, 10, 11, 12, 13, or 14 days after administration of the modified SC-beta cells.

448. The method of any of claims 430-444, wherein the one or more immunosuppressive agents are or have been administered to the subject at least 1 week, 2 weeks, 3 weeks, 4 weeks, 5 weeks, 6 weeks, 7 weeks, 8 weeks, 9 weeks, 10 weeks, or more, after administration of the modified SC-beta cells.

449. The method of any of claims 430-444, wherein the one or more immunosuppressive agents are or have been administered to the subject on the same day as the first administration of the modified SC-beta cells.

450. The method of any of claims 430-444, wherein the one or more immunosuppressive agents are or have been administered to the subject after administration of the modified SC-beta cells.

451. The method of any of claims 430-444, wherein the one or more immunosuppressive agents are or have been administered to the subject after administration of a first and/or second administration of the modified SC-beta cells.

452. The method of any of claims 430-444, wherein the one or more immunosuppressive agents are or have been administered to the subject prior to administration of a first and/or second administration of the modified SC-beta cells.

453. The method of any of claims 430-444, wherein the one or more immunosuppressive agents are or have been administered to the subject at least 1, 2, 3, 4, 5, 6, 7, 8, 9, 10, 11, 12, 13, or 14 days prior to administration of a first and/or second administration of the modified SC-beta cells.

454. The method of any of claims 430-444, wherein the one or more immunosuppressive agents are or have been administered to the subject at least 1 week, 2 weeks, 3 weeks, 4 weeks, 5 weeks, 6 weeks, 7 weeks, 8 weeks, 9 weeks, 10 weeks or more prior to administration of a first and/or second administration of the modified SC-beta cells.

455. The method of any of claims 430-444, wherein the one or more immunosuppressive agents are or have been administered to the subject at least 1, 2, 3, 4, 5, 6, 7, 8, 9, 10, 11, 12, 13, or 14 days after administration of a first and/or second administration of the modified SC-beta cells.

456. The method of any of claims 430-444, wherein the one or more immunosuppressive agents are or have been administered to the subject at least 1 week, 2 weeks, 3 weeks, 4 weeks, 5 weeks, 6 weeks, 7 weeks, 8 weeks, 9 weeks, 10 weeks, or more, after administration of a first and/or second administration of the modified SC-beta cells.

457. The method of any of claims 430-456, wherein the one or more immunosuppressive agents are administered at a lower dosage compared to the dosage of one or more immunosuppressive agents administered to reduce immune rejection of immunogenic cells that do not comprise the modifications of the modified SC-beta cells.

458. The method of any of claims 430-457, wherein the modified SC-beta cell is capable of controlled killing of the modified SC-beta cell.

459. The method of any of claims 430-458, wherein the modified SC-beta cell comprises a safety switch.

460. The method of claim 459, wherein the safety switch induces controlled cell death in the presence of a drug or prodrug, or upon activation by a selective exogenous compound.

461. The method of any of claims 459-460, wherein the safety switch is a system wherein upon activation, cells downregulate expression of the one or more tolerogenic factors and/or upregulate expression of one or more immune signaling molecules thereby marking the cell for elimination by the host immune system.

462. The method of claim 461, wherein the one or more tolerogenic factors are selected from the group consisting of CD16, CD24, CD35, CD39, CD46, CD47, CD52, CD55, CD59, CD64, CD200, CCL22, CTLA4-Ig, C1 inhibitor, FASL, IDO1, HLA-C, HLA-E, HLA-E heavy chain, HLA-G, IL-10, IL-35, PD-L1, SERPINB9, CCL21, MFGE8, DUX4, B2M-HLA-E, CD27, IL-39, CD16 Fc Receptor, IL15-RF, H2-M3 (HLA-G), A20/TNFAIP3, CR1, HLA-F, and MANF.

463. The method of claim 461 or claim 462, wherein the one or more immune signaling molecules are selected from the group consisting of B2M, HLA-A, HLA-B, HLA-C, HLA-D, HLA-E, RFXANK, CIITA, CTLA-4, PD-1, RAET1E/ULBP4, RAET1G/ULBP5, RAET1H/ULBP2, RAET1/ULBP1, RAET1L/ULBP6, RAET1N/ULBP3, and other ligands of NKG2D.

464. The method of claim 459 or claim 460, wherein the safety switch is an inducible protein capable of inducing apoptosis of the modified SC-beta cell.

465. The method of claim 464, wherein the inducible protein capable of inducing apoptosis of the modified SC-beta cell is a caspase protein.

466. The method of claim 465, wherein the caspase protein is caspase 9.

467. The method of any of claims 459-460, wherein the safety switch is a suicide gene.

468. The method of claim 467, wherein the suicide gene is selected from the group consisting of cytosine deaminase (CyD), herpesvirus thymidine kinase (HSV-Tk), an inducible caspase 9 (iCaspase9), and rapamycin-activated caspase 9 (rapaCasp9).

469. The method of claim 467, wherein the suicide gene is selected from the group consisting of cytosine deaminase (CyD), herpesvirus thymidine kinase (HSV-Tk), an inducible caspase 9 (iCaspase9), and rapamycin-activated caspase 9 (rapaCasp9).

470. The method of any of claims 459-469, wherein the safety switch is activated to induce controlled cell death after the administration of the one or more immunosuppressive agents to the subject.

471. The method of any of claims 459-469, wherein the safety switch is activated to induce controlled cell death prior to the administration of the one or more immunosuppressive agents to the subject.

472. The method of any of claims 459-471, wherein the safety switch is activated to induce controlled cell death after the administration of the modified SC-beta cell to the subject.

473. The method of any of claims 459-472, wherein the safety switch is activated to induce controlled cell death in the event of cytotoxicity or other negative consequences to the subject.

474. The method of any of claims 459-473, comprising administering an agent that allows for depletion of a modified SC-beta cell of the population of modified SC-beta cells.

475. The method of claim 474, wherein the agent that allows for depletion of the modified SC-beta cell is an antibody that recognizes a protein expressed on the surface of the modified SC-beta cell.

476. The method of claim 475, wherein the antibody is selected from the group consisting of an antibody that recognizes CCR4, CD16, CD19, CD20, CD30, EGFR, GD2, HER1, HER2, MUC1, PSMA, and RQR8.

477. The method of claim 475, wherein the antibody is selected from the group consisting of mogamulizumab, AFM13, MOR208, obinutuzumab, ublituximab, ocaratuzumab, rituximab, rituximab-Rllb, tomuzotuximab, RO5083945 (GA201), cetuximab, Hul4.18K322A, Hul4.18-IL2, Hu3F8, dinituximab, c.60C3-Rllc, and biosimilars thereof.

478. The method of any of claims 417-429 and 474-477, comprising administering an agent that recognizes the one or more tolerogenic factors on the surface of the modified SC-beta cell.

479. The method of claim 478, wherein the modified SC-beta cell is engineered to express the one or more tolerogenic factors.

480. The method of claim 478 or claim 479, wherein the one or more tolerogenic factors is CD47.

481. The method of any of claims 417-480, further comprising administering one or more additional therapeutic agents to the subject.

482. The method of any of claims 417-481, wherein the subject has been administered one or more additional therapeutic agents.

483. The method of any of claims 417-482, further comprising monitoring the therapeutic efficacy of the method.

484. The method of any of claims 417-483, further comprising monitoring the prophylactic efficacy of the method.

485. The method of any of claims 417-484, wherein the method is repeated until a desired suppression of one or more disease symptoms occurs.

486. The modified SC-beta cell of any of claims 205-380, wherein the modified SC-beta cell comprises an exogenous polynucleotide encoding a safety switch.

487. The modified SC-beta cell of claim 486, wherein the safety switch is a system wherein upon activation, cells downregulate expression of the one or more tolerogenic factors and/or upregulate expression of one or more immune signaling molecules thereby marking the cell for elimination by the host immune system.

488. The modified SC-beta cell of claim 487, wherein the one or more tolerogenic factors are selected from the group consisting of CD16, CD24, CD35, CD39, CD46, CD47, CD52, CD55, CD59, CD64, CD200, CCL22, CTLA4-Ig, C1 inhibitor, FASL, IDO1, HLA-C, HLA-E, HLA-E heavy chain, HLA-G, IL-10, IL-35, PD-L1, SERPINB9, CCL21, MFGE8, DUX4, B2M-HLA-E, CD27, IL-39, CD16 Fc Receptor, IL15-RF, H2-M3 (HLA-G), A20/TNFAIP3, CR1, HLA-F, and MANF.

489. The modified SC-beta cell of claim 487 or claim 488, wherein the one or more immune signaling molecules are selected from the group consisting of B2M, HLA-A, HLA-B, HLA-C, HLA-D, HLA-E, RFXANK, CIITA, CTLA-4, PD-1, RAET1E/ULBP4, RAET1G/ULBP5, RAET1H/ULBP2, RAET1/ULBP1, RAET1L/ULBP6, RAET1N/ULBP3, and other ligands of NKG2D.

490. The modified SC-beta cell of any of claims 487-489, wherein the safety switch is a suicide gene.

491. The modified SC-beta cell of claim 490, wherein the suicide gene is selected from the group consisting of cytosine deaminase (CyD), herpesvirus thymidine kinase (HSV-Tk), an inducible caspase 9 (iCaspase9), and rapamycin-activated caspase 9 (rapaCasp9).

492. The modified SC-beta cell of any of claims 486-491, wherein the safety switch and genes associated with the safety switch are expressed from a bicistronic cassette integrated into the genome of the modified SC-beta cell.

493. The modified SC-beta cell any of claims 486-491, wherein the safety switch and the one or more tolerogenic factors are expressed from a bicistronic cassette integrated into the genome of the modified SC-beta cell.

494. The modified SC-beta cell of claim 492 or claim 493, wherein the bicistronic cassette is integrated by non-targeted insertion into the genome of the modified SC-beta cell, optionally by introduction of the exogenous polynucleotide into the cell using a lentiviral vector.

495. The modified SC-beta cell of claim 492 or 493, wherein the bicistronic cassette is integrated by targeted insertion into a target genomic locus of the cell, optionally wherein the targeted insertion is by nuclease-mediated gene editing with homology-directed repair.

496. The modified SC-beta cell of any of claims 487-495, wherein the one or more tolerogenic factors is CD47.

497. The method of any of claims 1-203, wherein the modified SC-beta cell comprises an exogenous polynucleotide encoding a safety switch.

498. The method of claim 97, wherein the safety switch is a system wherein upon activation, cells downregulate expression of the one or more tolerogenic factors and/or upregulate expression of one or more immune signaling molecules thereby marking the cell for elimination by the host immune system.

499. The method of claim 98, wherein the one or more tolerogenic factors are selected from the group consisting of CD16, CD24, CD35, CD39, CD46, CD47, CD52, CD55, CD59, CD64, CD200, CCL22, CTLA4-Ig, C1 inhibitor, FASL, IDO1, HLA-C, HLA-E, HLA-E heavy chain, HLA-G, IL-10, IL-35, PD-L1, SERPINB9, CCL21, MFGE8, DUX4, B2M-HLA-E, CD27, IL-39, CD16 Fc Receptor, IL15-RF, H2-M3 (HLA-G), A20/TNFAIP3, CR1, HLA-F, and MANF.

500. The method of claim 97 or claim 98, wherein the one or more immune signaling molecules are selected from the group consisting of B2M, HLA-A, HLA-B, HLA-C, HLA-D, HLA-E, RFXANK, CIITA, CTLA-4, PD-1, RAET1E/ULBP4, RAET1G/ULBP5, RAET1H/ULBP2, RAET1/ULBP1, RAET1L/ULBP6, RAET1N/ULBP3, and other ligands of NKG2D.

501. The method of claim 97, wherein the safety switch is a suicide gene.

502. The method of claim 501, wherein the suicide gene is selected from the group consisting of cytosine deaminase (CyD), herpesvirus thymidine kinase (HSV-Tk), an inducible caspase 9 (iCaspase9), and rapamycin-activated caspase 9 (rapaCasp9).

503. The method of any of claims 497-502, wherein the safety switch and genes associated with the safety switch are expressed from a bicistronic cassette integrated into the genome of the modified SC-beta cell.

504. The method of any of claims 497-502, wherein the safety switch and the one or more tolerogenic factors are expressed from a bicistronic cassette integrated into the genome of the modified SC-beta cell.

505. The method of claim 503 or claim 504, wherein the bicistronic cassette is integrated by non-targeted insertion into the genome of the modified SC-beta cell.

506. The method of claim 503 or claim 504, wherein the bicistronic cassette is integrated by targeted insertion into a target genomic locus of the modified SC-beta cell.

507. The method of any of claims 498-506, wherein the one or more tolerogenic factors is CD47.

508. The composition of any of claims 204 and 381-416, wherein modified SC-beta cells of the population of modified SC-beta cells comprise an exogenous polynucleotide encoding a safety switch.

509. The composition of claim 508, wherein the safety switch is a system wherein upon activation, cells downregulate expression of the one or more tolerogenic factors and/or upregulate expression of one or more immune signaling molecules thereby marking the cell for elimination by the host immune system.

510. The composition of claim 509, wherein the one or more tolerogenic factors are selected from the group consisting of CD16, CD24, CD35, CD39, CD46, CD47, CD52, CD55, CD59, CD64, CD200, CCL22, CTLA4-Ig, C1 inhibitor, FASL, IDO1, HLA-C, HLA-E, HLA-E heavy chain, HLA-G, IL-10, IL-35, PD-L1, SERPINB9, CCL21, MFGE8, DUX4, B2M-HLA-E, CD27, IL-39, CD16 Fc Receptor, IL15-RF, H2-M3 (HLA-G), A20/TNFAIP3, CR1, HLA-F, and MANF.

511. The composition of claim 509 or claim 510, wherein the one or more immune signaling molecules are selected from the group consisting of B2M, HLA-A, HLA-B, HLA-C, HLA-D, HLA-E, RFXANK, CIITA, CTLA-4, PD-1, RAET1E/ULBP4, RAET1G/ULBP5, RAET1H/ULBP2, RAET1/ULBP1, RAET1L/ULBP6, RAET1N/ULBP3, and other ligands of NKG2D.

512. The composition of claim 508, wherein the safety switch is a suicide gene.

513. The composition of claim 512, wherein the suicide gene is selected from the group consisting of cytosine deaminase (CyD), herpesvirus thymidine kinase (HSV-Tk), an inducible caspase 9 (iCaspase9), and rapamycin-activated caspase 9 (rapaCasp9).

514. The composition of any of claims 508-513, wherein the safety switch and genes associated with the safety switch are expressed from a bicistronic cassette integrated into the genome of modified SC-beta cells of the population of modified SC-beta cells.

515. The composition of claim any of claims 508-513, wherein the safety switch and the exogenous CD47 are expressed from a bicistronic cassette integrated into the genome of the modified SC-beta cell.

516. The composition of any of claims 508-515, wherein the bicistronic cassette is integrated by non-targeted insertion into the genome, optionally by introduction of the exogenous polynucleotide into modified SC-beta cells of the population of modified SC-beta cells using a lentiviral vector.

517. The composition of any of claims 508-515, wherein the bicistronic cassette is integrated by targeted insertion into a target genomic locus of modified SC-beta cells of the population of modified SC-beta cells, optionally wherein the targeted insertion is by nuclease-mediated gene editing with homology-directed repair.


SEQUENCE LISTING (37 sequences)

SEQ ID NO: 1 - protein, 305 aa, Homo sapiens
QLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 2 - protein, 323 aa, Homo sapiens
MWPLVAALLLGSACCGSAQLLFNKTKSVEFTFCNDTVVIPCFVTNMEAQNTTEVYVKWKFKGRDIYTFDGALNKSTVPTDFSSAKIEVSQLLKGDASLKMDKSDAVSHTGNYTCEVTELTREGETIIELKYRVVSWFSPNENILIVIFPIFAILLFWGQFGIKTLKYRSGGMDEKTIALLVAGLVITVIVIVGAILFVPGEYSLKNATGLGLIVTSTGILILLHYYVFSTAIGLTSFVIAILVIQVIAYILAVVGLSLCIAACIPMHGPLLISGLSILALAQLLGLVYMKFVASNQKTIQPPRKAVEEPLNAFKESKGMMNDE

SEQ ID NO: 3 - protein, 358 aa, Homo sapiens
CEEPPTFEAMELIGKPKPYYEIGERVDYKCKKGYFYIPPLATHTICDRNHTWLPVSDDACYRETCPYIRDPLNGQAVPANGTYEFGYQMHFICNEGYYLIGEEILYCELKGSVAIWSGKPPICEKVLCTPPPKIKNGKHTFSEVEVFEYLDAVTYSCDPAPGPDPFSLIGESTIYCGDNSVWSRAAPECKVVKCRFPVVENGKQISGFGKKFYYKATVMFECDKGFYLDGSDTIVCDSNSTWDPPVPKCLKVLPPSSTKPPALSHSVSTSSTTKSPASSASGPRPTYKPPVSNYPGYPKPEEGILDSLDVWVIAVIVIAIVVGVAVICVVPYRYLQRRKKKGTYLTDETHREVKFTSL

SEQ ID NO: 4 - protein, 392 aa, Homo sapiens [sequence omitted]

SEQ ID NO: 5 - protein, 103 aa, Homo sapiens
LQCYNCPNPTADCKTAVNCSSDFDACLITKAGLQVYNKCWKFEHCNFNDVTTRLRENELTYYCCKKDLCNFNEQLENGGTSLSEKTVLLLVTPFLAAAWSLHP

SEQ ID NO: 6 - protein, 128 aa, Homo sapiens
MGIQGGSVLFGLLLVLAVFCHSGHSLQCYNCPNPTADCKTAVNCSSDFDACLITKAGLQVYNKCWKFEHCNFNDVTTRLRENELTYYCCKKDLCNFNEQLENGGTSLSEKTVLLLVTPFLAAAWSLHP

SEQ ID NO: 7 - protein, 77 aa, Homo sapiens
LQCYNCPNPTADCKTAVNCSSDFDACLITKAGLQVYNKCWKFEHCNFNDVTTRLRENELTYYCCKKDLCNFNEQLEN

SEQ ID NO: 8 - protein, 347 aa, Homo sapiens
DCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQATRSTPVSRTTKHFHETTPNKGSGTTSGTTRLLSGHTCFTLTGLLGTLVTMGLLT

SEQ ID NO: 9 - protein, 381 aa, Homo sapiens
MTVARPSVPAALPLLGELPRLLLLVLLCLPAVWGDCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQATRSTPVSRTTKHFHETTPNKGSGTTSGTTRLLSGHTCFTLTGLLGTLVTMGLLT

SEQ ID NO: 10 - protein, 319 aa, Homo sapiens
DCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQKTTTKTTTPNAQATRSTPVSRTTKHFHETTPNKGSGTTS

SEQ ID NO: 11 - protein, 18 aa, synthetic construct
EGRGSLLTCGDVEENPGP

SEQ ID NO: 12 - protein, 24 aa, synthetic construct
LEGGGEGRGSLLTCGDVEENPGPR

SEQ ID NO: 13 - protein, 22 aa, synthetic construct
GSGATNFSLLKQAGDVEENPGP

SEQ ID NO: 14 - protein, 19 aa, synthetic construct
ATNFSLLKQAGDVEENPGP

SEQ ID NO: 15 - protein, 20 aa, synthetic construct
QCTNYALLKLAGDVESNPGP

SEQ ID NO: 16 - protein, 22 aa, synthetic construct
VKQTLNFDLLKLAGDVESNPGP

SEQ ID NO: 17 - protein, 21 aa, synthetic construct
GSGEGRGSLLTCGDVEENPGP

SEQ ID NO: 18 - protein, 23 aa, synthetic construct
AAGSGEGRGSLLTCGDVEENPGP

SEQ ID NO: 19 - RNA, 12 nt, synthetic construct
  source 1..12: mol_type = other RNA
gttttagagcta

SEQ ID NO: 20 - RNA, 63 nt, synthetic construct
  source 1..63: mol_type = other RNA
tagcaagttaaaataaggctagtccgttatcaacttgaaaaagtggcaccgagtcggtgcttt

SEQ ID NO: 21 - skipped

SEQ ID NO: 22 - RNA, 20 nt, synthetic construct
  source 1..20: mol_type = other RNA
ttccctcccgaacagttaac

SEQ ID NO: 23 - RNA, 20 nt, synthetic construct
  source 1..20: mol_type = other RNA
ccgggctgtctgtactcttc

SEQ ID NO: 24 - RNA, 20 nt, synthetic construct
  source 1..20: mol_type = other RNA
cctcaccgacgagattgtga

SEQ ID NO: 25 - RNA, 20 nt, synthetic construct
  source 1..20: mol_type = other RNA
atgtgaccgtagaagatgaa

SEQ ID NO: 26 - RNA, 20 nt, synthetic construct
  source 1..20: mol_type = other RNA
taaagtccgttcatcttcta

SEQ ID NO: 27 - RNA, 20 nt, synthetic construct
  source 1..20: mol_type = other RNA
cacatccttcacaatctcgt

SEQ ID NO: 28 - RNA, 20 nt, synthetic construct
  source 1..20: mol_type = other RNA
agagtctctcagctggtaca

SEQ ID NO: 29 - RNA, 20 nt, synthetic construct
  source 1..20: mol_type = other RNA
tctctccatgtgcagtagga

SEQ ID NO: 30 - RNA, 20 nt, synthetic construct
  source 1..20: mol_type = other RNA
ctggatgtcggaggagtacg

SEQ ID NO: 31 - RNA, 20 nt, synthetic construct
  source 1..20: mol_type = other RNA
gtctccggaaactcgaggtg

SEQ ID NO: 32 - RNA, 20 nt, synthetic construct
  source 1..20: mol_type = other RNA
acagtgtagacttgattgac

SEQ ID NO: 33 - RNA, 20 nt, synthetic construct
  source 1..20: mol_type = other RNA
cgtgagtaaacctgaatctt

SEQ ID NO: 34 - RNA, 20 nt, synthetic construct
  source 1..20: mol_type = other RNA
gatattggcataagcctccc

SEQ ID NO: 35 - RNA, 20 nt, synthetic construct
  source 1..20: mol_type = other RNA
accccacagtggggccacta

SEQ ID NO: 36 - RNA, 20 nt, synthetic construct
  source 1..20: mol_type = other RNA
tgttggaaggatgaggaaat

SEQ ID NO: 37 - RNA, 20 nt, synthetic construct
  source 1..20: mol_type = other RNA
tcactatgctgccgcccagt